(19) United States
(12) Patent Application Publication
(10) Pub. No.: US 2011/0034469 A1
(43) Pub. Date: Feb. 10, 2011

(54) Heterocyclic Compound

(75) Inventors: Kentaro HASHIMOTO, Tsukuba-shi (JP); Tomoyasu ISHIKAWA, Tsukuba-shi (JP); Yuya OGURO, Tsukuba-shi (JP)

Correspondence Address: HOGAN LOVELLS US LLP;IP GROUP, COLUMBIA SQUARE, 555 THIRTEENTH STREET, N.W., WASHINGTON, DC, 20004, US

(73) Assignee: TAKEDA PHARMACEUTICAL COMPANY LIMITED, Osaka (JP)

(21) Appl. No.: 12/849,745

(22) Filed: Aug. 3, 2010

(30) Foreign Application Priority Data
Aug. 4, 2009 (JP) 181933/2009
Jan. 8, 2010 (JP) 003403/2010

(51) Int. Cl. A61K 31/4985; A61P 35/00; C07D 471/04

(52) U.S. Cl. 514/249; 544/349

(57) ABSTRACT

The present invention provides a compound represented by the formula

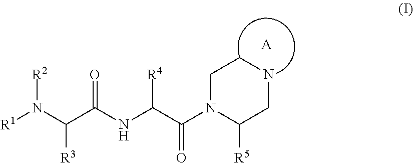

wherein each symbol is as defined in the specification, or a salt thereof. The compound of the present invention shows a strong IAP antagonistic activity.

### TECHNICAL FIELD

The present invention relates to a heterocyclic compound having an antagonistic activity against inhibitor of apoptosis proteins (sometimes to be abbreviated as IAP in the present specification), which is useful for the prophylaxis or treatment of cancer and the like, and use thereof.

### BACKGROUND OF THE INVENTION

Apoptosis or managed or controlled cell death (programmed cell death) plays an important role for the development of the body and maintenance of homeostasis, and collapse of apoptosis (cell death) signal is deeply involved in various diseases such as cancer, autoimmune diseases, neurodegenerative disease, inflammatory disease and the like (non-patent document 1). An important factor in apoptosis (cell death) is caspase, which is a serine protease involved in various protein decomposition as an effector of apoptosis. In many types of cancer, it survives and grows by suppressing caspase function via various signal molecules to acquire apoptosis (cell death) resistance.

The inhibitor of apoptosis proteins (IAP) is a protein group that suppresses apoptosis by being directly bound to caspase to suppress its function. IAP is identified to include proteins having a BIR domain as a common structure, and XIAP, cIAP-1, cIAP-2, ML-IAP, Survivin and the like have been reported (non-patent document 2).

Expression of IAP has been reported to be promoted in many cancers, and positively correlate with malignant alteration of cancer and poor prognosis. Recently, it has been clarified that Smac (DIABLO), which is a protein released from mitochondria in response to various cell death signals to induce apoptosis (cell death), is bound to the binding site of IAP proteins such as XIAP, cIAP and the like and releases Caspases from suppression, whereby strong apoptosis (cell death) is induced. In addition, an apoptosis induction activity of Smac has also been reported, which includes binding to IAP proteins such as XIAP, cIAP and the like to induce self-ubiquitination and degradation of the IAP proteins. Therefore an IAP antagonist, which is a low-molecular-weight compound mimicking the Smac peptide site important for binding with IAP, can be a promising therapeutic drug for cancer that induces apoptosis (cell death).

Since N-terminal of Smac peptide is important for the binding with IAP protein, IAP antagonist drugs of low-molecular-weight compounds that mimic N terminal-alanine•valine•proline•isoleucine (AVPI) site are known, and proline derivatives (patent document 1), piperazine-2-carboxylic acid derivatives (patent document 6), pyrrolidine derivatives (patent document 10), octahydrocyclopentapyrrole derivatives (patent document 2), octahydropyrrolopyridine derivatives (patent document 3) and the like have been reported. Additionally, the compounds described in patent documents 4, 5 and 7-9 have been reported as low-molecular-weight IAP inhibitors.

As a derivative relating to the above, compounds acting on a melanocortin receptor have also been reported (patent documents 14-17).

To the present, however, IAP antagonist has never been placed in the market as a therapeutic drug for cancer.

### PRIOR ART

### Patent Documents

### Patent Document 1: WO 2004/005248

### Patent Document 2: WO 2005/094818

### Patent Document 3: WO 2005/097791

### Patent Document 4: WO 2006/017295

### Patent Document 5: WO 2006/069063

### Patent Document 6: WO 2006/113376

### Patent Document 7: WO 2006/128455

### Patent Document 8: WO 2007/106192

### Patent Document 9: WO 2007/136921

### Patent Document 10: WO 2008/016893

### Patent Document 11: WO 2008/045905

### Patent Document 12: WO 2008/079735

### Patent Document 13: WO 2008/144925

### Patent Document 14: WO 02/079146

### Patent Document 15: WO 2004/037797

### Patent Document 16: WO 2005/047251

### Patent Document 17: WO 2005/047253

### Non-Patent Document

### Non-patent Document 1: Science 267 (1995) 1456-1462

Non-patent Document 2: J. Clin. Invest. 115 (2005) 2673-2678

### SUMMARY OF THE INVENTION

### Problems to be Solved by the Invention

An IAP antagonistic (inhibitory) drug superior in the efficacy expression, pharmacokinetics, solubility, interaction with other pharmaceutical products, safety (toxicity), stability and the like is expected to show a therapeutically superior effect. At present, however, such drug sufficiently satisfying them has not been found. Therefore, an object of the present invention is to provide a compound superior in the above-mentioned points and sufficiently satisfactory as a pharmaceutical product.

### Means of Solving the Problems

The present inventors have conducted intensive studies in an attempt to solve the aforementioned problems and found that a compound represented by the following formula has a superior IAP antagonistic (inhibitory) activity, which resulted in the completion of the present invention.

Accordingly, the present invention provides the following:

[1] A compound represented by the formula (I):

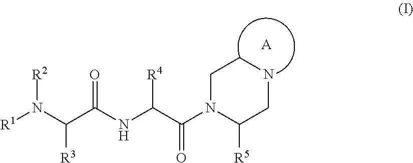

wherein
ring A is a nitrogen-containing heterocycle optionally having substituent(s);
R¹ is a hydrogen atom, a chain aliphatic hydrocarbon group optionally having substituent(s), C(═O)—R¹¹, —C(═O)—O—R¹¹, —N(R¹²)—R¹¹ or —C(═O)—N(R¹²)—R¹¹;
R¹¹ is a chain aliphatic hydrocarbon group optionally having substituent(s);
R¹² is a′ hydrogen atom or a chain aliphatic hydrocarbon group optionally having substituent(s);
R² is a hydrogen atom, a chain aliphatic hydrocarbon group optionally having substituent(s), —C(═O)—R²¹, —C(═O)—O—R²¹ or —C(═O)—N(R²²)—R²¹;
R²¹ is a chain aliphatic hydrocarbon group optionally having substituent(s);
R²² is a hydrogen atom or a chain aliphatic hydrocarbon group optionally having substituent(s);
R³ is a hydrogen atom or a chain aliphatic hydrocarbon group optionally having substituent(s); or
R¹ and R² in combination or R¹ and R³ in combination form a O-5 to 7-membered nitrogen-containing non-aromatic heterocycle optionally having substituent(s);
R⁴ is a cyclic group optionally having substituent(s) or a chain aliphatic hydrocarbon group optionally having substituent(s);
R⁵ is a cyclic group optionally having substituent(s), —C(═O)NR⁵¹—X¹—R⁵², —C(═S)NR⁵¹—X¹—R⁵², —X²—R⁵², —X²—NR⁵¹—C(═O)—R⁵³ or —C(═NR⁵⁴)—NR⁵¹—X¹—R⁵²;
X¹ is a bond or C₁₋₆ alkylene optionally having substituent(s);
X² is C₁₋₆ alkylene optionally having substituent(s);
R⁵¹ is a hydrogen atom, a chain aliphatic hydrocarbon group optionally having substituent(s) or a cyclic group optionally having substituent(s);
R⁵² is a cyclic group optionally having substituent(s);
R⁵³ is a chain aliphatic hydrocarbon group optionally having substituent(s) or a cyclic group optionally having substituent(s); and
R⁵⁴ is a hydrogen atom or a chain aliphatic hydrocarbon group optionally having substituent(s),
or a salt thereof (hereinafter to be referred to as “compound (I)” in the present specification, compound (I) conceptionally includes the following compound (II));
[2] the compound of the above-mentioned [1], wherein ring A is a nitrogen-containing heterocycle having C₁₋₆ alkoxy, or a salt thereof;
[3] the compound of the above-mentioned [1], wherein ring A is an unsubstituted nitrogen-containing heterocycle, or a salt thereof;
[4] the compound of the above-mentioned [1], wherein ring A is a 4- to 7-membered monocyclic nitrogen-containing non-aromatic heterocycle optionally having 1 to 5 substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl;
(e) C₃₋₈ cycloalkyloxy;
(f) C₆₋₁₀ aryloxy;
(g) aralkyloxy; and
(h) oxo,
   or a salt thereof;
   [5] the compound of the above-mentioned [1], wherein ring A is pyrrolidine optionally having 1 to 5 substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl;
(e) C₃₋₈ cycloalkyloxy;
(f) C₆₋₁₀ aryloxy;
(g) C₇₋₁₃ aralkyloxy; and
(h) oxo,
   or a salt thereof;
   [6] the compound of any of the above-mentioned [1] to [5], wherein R¹ is C₁₋₆ alkyl, or a salt thereof;
   [7] the compound of any of the above-mentioned [1] to [6], wherein R² is a hydrogen atom, or a salt thereof;
   [8] the compound of any of the above-mentioned [1] to [7],

wherein R³ is C₁₋₆ alkyl, or a salt thereof;

[9] the compound of any of the above-mentioned [1] to [8], wherein R⁴ is a cyclic group having halogen atom(s), or a salt thereof;
[10] the compound of any of the above-mentioned [1] to [8]; wherein R⁴ is an unsubstituted cyclic group, or a salt thereof;
[11] the compound of any of the above-mentioned [1] to [8], wherein R⁴ is C₃₋₈ cycloalkyl optionally having 1 to 3 substituents selected from
(a) a halogen atom,
(b) hydroxy,
(c) C₁₋₆ alkoxy-carbonyl optionally having one C₆₋₁₀ aryl, and
(d) oxo,
   or a salt thereof;
   [12] the compound of any of the above-mentioned [1] to [11], wherein R⁵ is
   (1) a 5- to 7-membered monocyclic aromatic heterocyclic group optionally having one substituent selected from
(a) C₆₋₁₀ aryl optionally having 1 to 3 halogen atoms,
(b) C₆₋₁₀ aryl-carbonyl optionally having 1 to 3 substituents selected from a halogen atom, C₁₋₆ alkyl optionally having 1 to 3 halogen atoms, C₁₋₆ alkoxy and C₆₋₁₀ aryl,
(c) 5- to 7-membered monocyclic aromatic heterocyclyl-carbonyl optionally having 1 to 3 substituents selected from cyano and C₁₋₆ alkyl,
(d) 8- to 12-membered fused aromatic heterocyclyl-carbonyl,
(e) C₇₋₁₃ aralkyl optionally having 1 to 3 halogen atoms, and
(f) C₂₋₆ alkenyl-carbonyl optionally having one C₆₋₁₀ aryl;
   (2) a 8- to 12-membered fused aromatic heterocyclic group optionally having one substituent selected from
(a) C₆₋₁₀ aryl optionally having 1 to 3 halogen atoms,
(b) C₆₋₁₀ aryl-carbonyl optionally having 1 to 3 substituents selected from a halogen atom, C₁₋₆ alkyl optionally having 1 to 3 halogen atoms, C₁₋₆ alkoxy and C₆₋₁₀ aryl,
(c) 5- to 7-membered monocyclic aromatic heterocyclyl-carbonyl optionally having 1 to 3 substituents selected from cyano and C₁₋₆ alkyl,
(d) 8- to 12-membered fused aromatic heterocyclyl-carbonyl,
(e) C₇₋₁₃ aralkyl optionally having 1 to 3 halogen atoms, and
(f) C₂₋₆ alkenyl-carbonyl optionally having one C₅₋₁₀ aryl;
   (3) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring, which optionally has 1 to 3 substituents selected from hydroxy, a halogen atom, C₁₋₆ alkyl-carbonyloxy and oxo;
(b) a monovalent group derived from a fused ring formed by condensation of tetrahydropyran or tetrahydrofuran and a benzene ring, which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group optionally having one C₆₋₁₀ aryl; or
(e) dihydroindolyl, and
X′ is
(a) a bond, or
(b) C₁₋₆ alkylene optionally having 1 to 3 substituents selected from C₆₋₁₀ aryl and a halogen atom;
   (4) —X²—R⁵² wherein
R⁵² is a 8- to 12-membered fused non-aromatic heterocyclic group, and
X² is C₁₋₆ alkylene; or
   (5) —X²—NR⁵¹R⁵³ wherein
R⁵¹ is a hydrogen atom,
R⁵³ is a monovalent group derived from a fused ring formed by condensation of C₃₋₈ cycloalkane and a benzene ring, and
X² is C₁₋₆ alkylene;
   or a salt thereof;
   [13] the compound of any of the above-mentioned [1] to [11], wherein R⁵ is —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring, which optionally has 1 to 3 substituents selected from hydroxy, a halogen atom, C₁₋₆ alkyl-carbonyloxy and oxo;
(b) a monovalent group derived from a fused ring formed by condensation of tetrahydropyran or tetrahydrofuran and a benzene ring, which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group optionally having one C₆₋₁₀ aryl; or
(e) dihydroindolyl, and
X¹ is
(a) a bond, or
(b) C₁₋₈ alkylene optionally having 1 to 3 substituents selected from C₆₋₁₀ aryl and a halogen atom;
   or a salt thereof;
   [14] the compound the above-mentioned [1], wherein
   ring A is a 4- to 7-membered monocyclic nitrogen-containing non-aromatic heterocycle optionally having substituent(s) or a 6- to 12-membered fused nitrogen-containing non-aromatic heterocycle optionally having substituent(s),
   R¹ is a hydrogen atom, C₁₋₈ alkyl optionally having substituent(s) or —OR¹¹ wherein R¹¹ is defined as in the above-mentioned [1],
   R² is a hydrogen atom or C₁₋₈ alkyl optionally having substituent(s),
   R³ is a hydrogen atom, C₁₋₈ alkyl optionally having substituent(s) or C₃₋₈ cycloalkyl optionally having substituent(s), or
   R¹ and R³ in combination form, together with the adjacent nitrogen atom and carbon atom, a 4- to 7-membered nitrogen-containing non-aromatic heterocycle optionally having substituent(s),

### R⁴ is

(1) C₃₋₈ cycloalkyl optionally having substituent(s),
(2) a 4- to 7-membered monocyclic non-aromatic heterocyclic group optionally having substituent(s),
(3) C₁₋₆ alkyl optionally having substituent(s), or
(4) C₆₋₁₀ aryl optionally having substituent(s),

### R⁵ is

(1) a 5- to 7-membered monocyclic aromatic heterocyclic group optionally having substituent(s),
(2) a 8- to 12-membered fused aromatic heterocyclic group optionally having substituent(s),
(3) —C(═O)NR⁵¹—X¹—R⁵² wherein R⁵¹, X¹ and R⁵² are defined as in the above-mentioned [1],
(4) —X²—R⁵² wherein R⁵² and X² are defined as in the above-mentioned [1], or
(5) —X²—NR⁵¹R⁵³ wherein R⁵¹, X² and R⁵³ are defined as in the above-mentioned [1],
or a salt thereof;
[15] a compound represented by the formula (II):

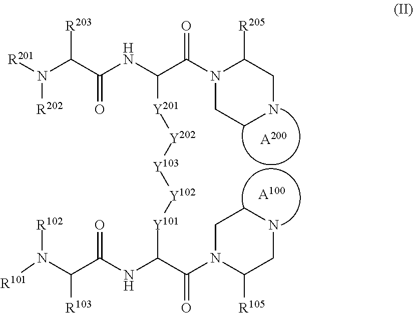

wherein
ring A¹⁰⁰ is a nitrogen-containing heterocycle optionally having substituent(s);
R¹⁰¹ is a hydrogen atom, a chain aliphatic hydrocarbon group optionally having substituent(s), —OR¹¹¹, —C(═O)—R¹¹¹, —C(═O)—O—R¹¹¹, —N(R¹¹²)—R¹¹¹ or —C(═O)—N(R¹¹²)—R¹¹¹;
R¹¹¹ is a chain aliphatic hydrocarbon group optionally having substituent (s);
R¹¹² is a hydrogen atom or a chain aliphatic hydrocarbon group optionally having substituent(s);
R¹⁰² is a hydrogen atom, a chain aliphatic hydrocarbon group optionally having substituent(s), —C(═O)—R¹²¹, —C(═O)—O—R¹²¹ or —C(═O)—N(R¹²²)—R¹²¹;
R¹²¹ is a chain aliphatic hydrocarbon group optionally having substituent(s);
R¹²² is a hydrogen atom or a chain aliphatic hydrocarbon group optionally having substituent(s);
R¹⁰³ is a hydrogen atom or a chain aliphatic hydrocarbon group optionally having substituent(s); or
R¹⁰¹ and R¹⁰² in combination or R¹⁰¹ and R¹⁰³ in combination form a 4- to 7-membered nitrogen-containing non-aromatic heterocycle optionally having substituent(s);
R¹⁰⁵ is a cyclic group optionally having substituent(s), —C(═O)—NR¹⁵¹—X¹⁰¹—R¹⁵², —C(═S)—NR¹⁵¹—X¹⁰¹—R¹⁵², —X¹⁰²—R¹⁵², —X¹⁰²—NR¹⁵¹R¹⁵³, X¹⁰²—NR¹⁵¹—C(═O)—R¹⁵³ or —C(═NR¹⁵⁴)—NR¹⁵¹—X¹⁰¹—R¹⁵²;
X¹⁰¹ is a bond or C₁₋₆ alkylene optionally having substituent(s);
X¹⁰² is C₁₋₆ alkylene optionally having substituent(s);
R¹⁵¹ is a hydrogen atom, a chain aliphatic hydrocarbon group optionally having substituent(s) or a cyclic group optionally having substituent(s);
R¹⁵² is a cyclic group optionally having substituent(s);
R¹⁵³ is a chain aliphatic hydrocarbon group optionally having substituent(s) or a cyclic group optionally having substituent(s);
R¹⁵⁴ is a hydrogen atom or a chain aliphatic hydrocarbon group optionally having substituent(s);
—Y¹⁰¹— is a divalent chain aliphatic hydrocarbon group optionally having substituent(S) or a divalent cyclic group optionally having substituent(s);
—Y¹⁰²— is a bond, —C(═O)—, —C(═O)—O—, —C(═O)—NR¹⁰⁶—, —O—, —O—C(═O)—, —O—C(═O)—NR¹⁰⁶—, —NR¹⁰⁶—, —NR¹⁰⁶—C(═O)—, —NR¹⁰⁶—C(═O)—O—, —NR¹⁰⁶—S(═O)₂—, —S—, —S(═O)—, —S(═O)₂— or —S(═O)₂—NR¹⁰⁶—;
R¹⁰⁶ is a hydrogen atom or a chain aliphatic hydrocarbon group optionally having substituent(s);
—Y¹⁰³— is a bond or a spacer having 1 to 20 atoms in the main chain;
ring A²⁰⁰ is a nitrogen-containing heterocycle optionally having substituent(s);
R²⁰¹ is a hydrogen atom, a chain aliphatic hydrocarbon group optionally having substituent(s), —OR²¹¹, —C(═O)—R²¹¹, —C(═O)—O—R²¹¹, —N(R²¹²)—R²¹¹ or —C(═O)—N(R²¹²)—R²¹¹;
R²¹¹ is a chain aliphatic hydrocarbon group optionally having substituent(s);
R²¹² is a hydrogen atom or a chain aliphatic hydrocarbon group optionally having substituent(s);
R²⁰² is a hydrogen atom, a chain aliphatic hydrocarbon group optionally having substituent(s), —C(═O)—R²²¹, —C(═O)—O—R²²¹ or —C(═O)—N(R²²²)—R²²¹;
R²²¹ is a chain aliphatic hydrocarbon group optionally having substituent(s);
R²²² is a hydrogen atom or a chain aliphatic hydrocarbon group optionally having substituent(s);
R²⁰³ is a hydrogen atom or a chain aliphatic hydrocarbon group optionally having substituent(s); or
R²⁰¹ and R²⁰² in combination or R²⁰¹ and R²⁰³ in combination form a 4- to 7-membered nitrogen-containing non-aromatic heterocycle optionally having substituent(s);
R²⁰⁵ is a cyclic group optionally having substituent(s), —C(═O)—NR²⁵¹—X²⁰¹—R²⁵², —C(═S)—NR²⁵¹—X²⁰¹—R²⁵², —X²⁰²—R²⁵², —X²⁰²—NR²⁵¹R²⁵³, —X²⁰²—NR²⁵¹—C(═O)—R²⁵³ or —C(═NR²⁵⁴)—NR²⁵¹—X²⁰¹;
X²⁰¹ is a bond or C₁₋₆ alkylene optionally having substituent(s);
X²⁰² is C₁₋₆ alkylene optionally having substituent(s);
R²⁵¹ is a hydrogen atom, a chain aliphatic hydrocarbon group optionally having substituent(s) or a cyclic group optionally having substituent(s);
R²⁵² is a cyclic group optionally having substituent(s);
R²⁵³ is a chain aliphatic hydrocarbon group optionally having substituent(s) or a cyclic group optionally having substituent(s);
R²⁵⁴ is a hydrogen atom or a chain aliphatic hydrocarbon group optionally having substituent(s);
—Y²⁰¹— is a divalent chain aliphatic hydrocarbon group optionally having substituent(s) or a divalent cyclic group optionally having substituent(s);
—Y²⁰²— is a bond, —C(═O)—, —C(═O)—O—, —C(═O)—NR²⁰⁶—, —O—, —O—C(═O)—, —O—C(═O)—NR²⁰⁶—, —NR²⁰⁶—, —NR²⁰⁶—C(═O)—, —NR²⁰⁶—C(═O)—O—, —NR²⁰⁶—S(═O)₂—, —S—, —S(═O)—, —S(═O)₂— or —S(═O)₂—NR²⁰⁶—;
R²⁰⁶ is a hydrogen atom or a chain aliphatic hydrocarbon group optionally having substituent(s), or a salt thereof (hereinafter to be referred to as “compound (II)” in the present specification);
[16] a compound which is (3S,7R,8aR)-2-{(2S)-2-cyclohexyl-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide, or a salt thereof;
[17] a compound which is (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide, or a salt thereof;
[18] a compound which is (3S,7R,8aR)—N-[(4R)-3,4-dihydro-2H-chromen-4-yl]-7-ethoxy-2-{(2S)-2-[(N-methyl-L-alanyl)amino]-2-phenylacetyl}octahydropyrrolo[1,2-a]pyrazine-3-carboxamide, or a salt thereof;
[19] a compound which is (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide, or a salt thereof;
[20] a compound which is (3S,8aR)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide, or a salt thereof;
[21] a compound which is (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide, or a salt thereof;
[22] a prodrug of the compound of any of the above-mentioned [1] to [21] or a salt thereof;
[23] a medicament comprising the compound of any of the above-mentioned [1] to [21] or a salt thereof or a prodrug thereof.
[24] the medicament of the above-mentioned [23], which is an antagonist of inhibitor of apoptosis proteins;
[25] the medicament of the above-mentioned [23], which is an agent for the prophylaxis or treatment of cancer;
[26] a method of antagonizing inhibitor of apoptosis proteins in a mammal, which comprises administering an effective amount of the compound of any of the above-mentioned [1] to [21] or a salt thereof or a prodrug thereof to the mammal;
[27] a method for the prophylaxis or treatment of cancer in a mammal, which comprises administering an effective amount of the compound of any of the above-mentioned [1] to [21] or a salt thereof or a prodrug thereof to the mammal;
[28] use of the compound of any of the above-mentioned [1] to [21] or a salt thereof or a prodrug thereof for the production of an antagonist of inhibitor of apoptosis proteins.
[29] use of the compound of any of the above-mentioned [1] to [21] or a salt thereof or a prodrug thereof for the production of an agent for the prophylaxis or treatment of cancer.

### EFFECT OF THE INVENTION

The compound of the present invention shows a superior IAP antagonistic action, and is also superior in pharmacokinetics, dissolution property, interaction with other pharmaceutical products, safety such as toxicity and the like, and stability. Therefore, a clinically useful prophylactic or therapeutic agent for IAP associated diseases (e.g., prophylactic or therapeutic agent for cancer, cancer growth inhibitor, cancer metastasis suppressive agent, apoptosis promoter) can be provided.

### DETAILED DESCRIPTION OF THE INVENTION

The “halogen atom” in the present specification means a fluorine atom, a chlorine atom, a bromine atom or an iodine atom.

The “C₁₋₆ alkyl” in the present specification means, for example, methyl, ethyl, propyl, isopropyl, butyl, isobutyl, sec-butyl, tert-butyl, pentyl, isopentyl, neopentyl, 1-s ethylpropyl, hexyl, isohexyl, 1,1-dimethylbutyl, 2,2-dimethylbutyl, 3,3-dimethylbutyl, 2-ethylbutyl or the like.

The “C₂₋₆ alkenyl” in the present specification means, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl or the like.

The “C₂₋₆ alkynyl” in the present specification means, for example, ethynyl, 1-propynyl, 2-propynyl, 1-butynyl, 2-butynyl, 3-butynyl, 1-pentynyl, 2-pentynyl, 3-pentynyl, 4-pentynyl, 1,1-dimethylprop-2-yn-1-yl, 1-hexynyl, 2-hexynyl, 3-hexynyl, 4-hexynyl, 5-hexynyl or the like.

The “C₁₋₆ alkoxy” in the present specification means, for example, methoxy, ethoxy, propoxy, isopropoxy, butoxy, isobutoxy, sec-butoxy, tert-butoxy, pentoxy, isopentoxy, hexoxy or the like.

The “C₁₋₆ alkyl-carbonyl” in the present specification means, for example, acetyl, ethylcarbonyl, propylcarbonyl, isopropylcarbonyl, butylcarbonyl, isobutylcarbonyl, sec-butylcarbonyl, tert-butylcarbonyl, pentylcarbonyl, hexylcarbonyl or the like.

The “C₁₋₆ alkoxy-carbonyl” in the present specification means, for example, methoxycarbonyl, ethoxycarbonyl, propoxycarbonyl, isopropoxycarbonyl, butoxycarbonyl, isobutoxycarbonyl, tert-butoxycarbonyl or the like.

The “C₃₋₈ cycloalkyl” in the present specification means, for example, cyclopropyl, cyclobutyl, cyclopentyl, cyclohexyl, cycloheptyl, cyclooctyl or the like.

The “C₃₋₈ cycloalkane” in the present specification means, for example, cyclopropane, cyclobutane, cyclopentane, cyclohexane, cycloheptane, cyclooctane or the like.

The “C₃₋₈ cycloalkane” in the present specification means, for example, cyclopropane, cyclobutane, cyclopentane, cyclohexane or the like.

The “C₃₋₈ cycloalkenyl” in the present specification means, for example, cyclopropenyl (e.g., 2-cyclopropen-1-yl), cyclobutenyl (e.g., 2-cyclobuten-1-yl), cyclopentenyl (e.g., 2-cyclopenten-1-yl, 3-cyclopenten-1-yl), cyclohexenyl (e.g., 2-cyclohexen-1-yl, 3-cyclohexen-1-yl) or the like.

The “C₆₋₁₀ aryl” in the present specification means, for example, phenyl, 1-naphthyl, 2-naphthyl or the like.

The “C₆₋₁₀ arene” in the present specification means, for example, benzene, naphthalene or the like.

The “C₇₋₁₃ aralkyl” in the present specification means, for example, benzyl, phenethyl, naphthylmethyl or the like.

The “heterocyclic group” in the present specification means an aromatic heterocyclic group or a non-aromatic heterocyclic group.

The “aromatic heterocyclic group” in the present specification means a monocyclic aromatic heterocyclic group or a fused aromatic heterocyclic group.

Examples of the monocyclic aromatic heterocyclic group include a 5- to 7-membered (preferably 5- or 6-membered) monocyclic aromatic heterocyclic group containing, as a ring-constituting atom besides carbon atoms, 1 to 4 hetero atoms selected from an oxygen atom, a sulfur atom and a nitrogen atom, for example, furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl) and triazinyl (e.g., 1,2,4-triazin-3-yl, 1,2,4-triazin-6-yl).

Examples of the fused aromatic heterocyclic group include a 8- to 12-membered fused aromatic heterocyclic group, specifically, a group derived from a fused ring formed by condensation of a ring corresponding to the above-mentioned 5- to 7-membered monocyclic aromatic heterocyclic group and C₆₋₁₀ arene; and a group derived from a fused ring formed by condensation of rings corresponding to the above-mentioned 5- to 7-membered monocyclic aromatic heterocyclic groups; for example, quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothienyl (e.g., 2-benzothienyl, 3-benzothienyl), benzoxazolyl (e.g., 2-benzoxazolyl), benzisoxazolyl (e.g., 3-benzisoxazolyl), benzothiazolyl (e.g., 2-benzothiazolyl), benzoisothiazolyl (e.g., 3-benzoisothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), thienopyridinyl (e.g., thieno[2,3-b]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl) and pyrazolotriazinyl (e.g., pyrazolo[5,1-c][1,2,4]triazin-3-yl).

The “5- to 12-membered aromatic heterocyclic group” in the present specification means the above-mentioned 5- to 7-membered monocyclic aromatic heterocyclic group or 8- to 12-membered fused aromatic heterocyclic group.

The “non-aromatic heterocyclic group” in the present specification means a monocyclic non-aromatic heterocyclic group or a fused non-aromatic heterocyclic group.

Examples of the monocyclic non-aromatic heterocyclic group include a 4- to 7-membered (preferably 5- or 6-membered) monocyclic non-aromatic heterocyclic group containing, as a ring-constituting atom besides carbon atoms, 1 to 4 hetero atoms selected from an oxygen atom, a sulfur atom (optionally oxidized) and a nitrogen atom, for example, azetidinyl (e.g., 1-azetidinyl, 2-azetidinyl), pyrrolidinyl (e.g., 1-pyrrolidinyl, 2-pyrrolidinyl), piperidyl (e.g., piperidino, 2-piperidyl, 3-piperidyl), morpholinyl (e.g., morpholino), thiomorpholinyl (e.g., thiomorpholino), piperazinyl (e.g., 1-piperazinyl, 2-piperazinyl, 3-piperazinyl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), pyranyl (e.g., 2-pyranyl, 4-pyranyl), tetrahydropyranyl (e.g., 2-tetrahydropyranyl, 3-tetrahydropyranyl, 4-tetrahydropyranyl), thiopyranyl (e.g., 4-thiopyranyl), dihydrothiopyranyl (e.g., dihydrothiopyran-3-yl, dihydrothiopyran-4-yl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-s oxidotetrahydrothiopyran-4-yl), 1,1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl), azepanyl (e.g., 1-azepanyl, 2-azepanyl, 3-azepanyl, 4-azepanyl), dihydropyridyl (e.g., dihydropyridin-1-yl, dihydropyridin-2-yl, dihydropyridin-3-yl, dihydropyridin-4-yl) and tetrahydropyridyl (e.g., tetrahydropyridin-1-yl, tetrahydropyridin-2-yl, tetrahydropyridin-3-yl, tetrahydropyridin-4-yl).

Examples of the fused non-aromatic heterocyclic group include a 8- to 12-membered fused non-aromatic heterocyclic group, specifically, a group derived from a fused ring formed by condensation of a ring corresponding to the above-mentioned 4- to 7-membered monocyclic non-aromatic heterocyclic group and C₆₋₁₀ arene; a group derived from a fused ring formed by condensation of rings corresponding to the above-mentioned 4- to 7-membered monocyclic non-aromatic heterocyclic groups; a group derived from a fused ring formed by condensation of a ring corresponding to the above-mentioned 4- to 7-membered monocyclic non-aromatic heterocyclic group and a ring corresponding to the above-mentioned 5- to 7-membered monocyclic aromatic heterocyclic group; and groups obtained by partial saturation of these groups; for example, dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydro-1-benzofuran-5-yl), tetrahydrobenzofuranyl (e.g., 4,5,6,7-tetrahydro-1-benzofuran-3-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), chromenyl (e.g., 4H-chromen-2-yl, 2H-chromen-3-yl), dihydrochromenyl (e.g., 3,4-dihydro-2H-chromen-2-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and azabicyclohexyl (e.g., 2-azabicyclo[3.1.0]hexan-3-yl).

The “4- to 12-membered non-aromatic heterocyclic group” in the present specification means the above-mentioned 4- to 7-membered monocyclic non-aromatic heterocyclic group or 8- to 12-membered fused non-aromatic heterocyclic group.

The “chain aliphatic hydrocarbon group” of the “chain aliphatic hydrocarbon group optionally having substituent(s)” in the present specification means, for example, C₁₋₆ alkyl, C₂₋₆ alkenyl or C₂₋₆ alkynyl.

Examples of the substituent that “chain aliphatic hydrocarbon group” optionally has include substituents selected from the following Substituent Group A. The number of the substituents is not particularly limited as long as it is a substitutable number, which is preferably 1 to 5, more preferably 1 to 3. When plural substituents are present, they may be the same or different.

### Substituent Group A.

(1) a halogen atom;
(2) cyano;
(3) nitro;
(4) hydroxy;
(5) carboxy;
(6) C₃₋₈ cycloalkyl optionally having 1 to 3 substituents selected from
(a) a halogen atom,
(b) hydroxy,
(c) C₁₋₆ alkyl optionally having 1 to 3 halogen atoms,
(d) C₁₋₆ alkoxy optionally having 1 to 3 halogen atoms, and
(e) oxo;
   (7) C₆₋₁₀ aryl optionally having 1 to 3 substituents selected from
(a) a halogen atom,
(b) hydroxy,
(c) C₁₋₆ alkyl optionally having 1 to 3 halogen atoms, and
(d) C₁₋₆ alkoxy optionally having 1 to 3 halogen atoms;
   (8) a 5- to 12-membered aromatic heterocyclic group optionally having 1 to 3 substituents selected from
(a) a halogen atom,
(b) hydroxy,
(c) C₁₋₆ alkyl optionally having 1 to 3 halogen atoms, and
(d) C₁₋₆ alkoxy optionally having 1 to 3 halogen atoms;
   (9) a 4- to 12-membered non-aromatic heterocyclic group optionally having 1 to 3 substituents selected from
(a) a halogen atom,
(b) hydroxy,
(c) C₁₋₆ alkyl optionally having 1 to 3 halogen atoms,
(d) C₁₋₆ alkoxy optionally having 1 to 3 halogen atoms, and
(e) oxo;
   (10) amino optionally having 1 or 2 substituents selected from
(a) C₁₋₆ alkyl optionally having 1 to 3 halogen atoms,
(b) C₁₋₆ alkyl-carbonyl optionally having 1 to 3 substituents selected from
   (i) a halogen atom, and
   (ii) C₆₋₁₀ aryl,
(c) C₁₋₆ alkoxy-carbonyl optionally having 1 to 3 substituents selected from
   (i) a halogen atom, and
   (ii) C₆₋₁₀ aryl,
(d) C₁₋₆ alkylsulfonyl (e.g., methylsulfonyl) optionally having 1 to 3 substituents selected from
   (i) a halogen atom, and
   (ii) C₆₋₁₀ aryl,
(e) C₆₋₁₀ arylsulfonyl (e.g., phenylsulfonyl),
(f) carbamoyl optionally having 1 or 2 C₁₋₆ alkyl optionally having 1 to 3 halogen atoms,
(g) a 5- to 12-membered aromatic heterocyclic group optionally having 1 to 3 substituents selected from
   (i) C₁₋₆ alkyl optionally having 1 to 3 halogen atoms,
   (ii) hydroxy,
   (iii) C₁₋₆ alkoxy optionally having 1 to 3 halogen atoms, and
   (iv) a halogen atom, and
(h) a 4- to 12-membered non-aromatic heterocyclic group optionally having 1 to 3 substituents selected from
   (i) C₁₋₆ alkyl optionally having 1 to 3 halogen atoms,
   (ii) hydroxy,
   (iii) C₁₋₆ alkoxy optionally having 1 to 3 halogen atoms,
   (iv) a halogen atom, and
   (v) oxo;
      (11) imino;
      (12) C₁₋₆ alkyl-carbonyl optionally having 1 to 3 halogen atoms;
      (13) C₁₋₆ alkoxy-carbonyl optionally having 1 to 3 substituents selected from
(a) a halogen atom,
(b) C₁₋₆ alkoxy,
(c) C₆₋₁₀ aryl,
(d) a 5- to 12-membered aromatic heterocyclic group optionally having 1 to 3 substituents selected from
   (i) C₁₋₆ alkyl optionally having 1 to 3 halogen atoms,
   (ii) hydroxy,
   (iii) C₁₋₆ alkoxy optionally having 1 to 3 halogen atoms, and
   (iv) a halogen atom, and
(e) a 4- to 12-membered non-aromatic heterocyclic group optionally having 1 to 3 substituents selected from
   (i) C₁₋₆ alkyl optionally having 1 to 3 halogen atoms,
   (ii) hydroxy,
   (iii) C₁₋₆ alkoxy optionally having 1 to 3 halogen atoms,
   (iv) a halogen atom, and
   (v) oxo;
      (14) C₁₋₆ alkylsulfonyl (e.g., methylsulfonyl, ethylsulfonyl, isopropylsulfonyl) optionally having 1 to 3 halogen atoms;
      (15) C₆₋₁₀ arylsulfonyl (e.g., phenylsulfonyl);
      (16) carbamoyl optionally having 1 or 2 C₁₋₆ alkyl optionally having 1 to 3 halogen atoms;
      (17) thiocarbamoyl optionally having 1 or 2 C₁₋₆ alkyl optionally having 1 to 3 halogen atoms;
      (18) sulfamoyl optionally having 1 or 2 C₁₋₆ alkyl optionally having 1 to 3 halogen atoms;
      (19) C₁₋₆ alkoxy optionally having 1 to 3 substituents selected from
(a) a halogen atom,
(b) carboxy,
(c) C₁₋₆ alkoxy,
(d) C₁₋₆ alkoxy-carbonyl optionally having 1 to 3 C₆₋₁₀ aryl,
(e) amino optionally having 1 or 2 substituents selected from C₁₋₆ alkyl and C₁₋₆ alkoxy-carbonyl,
(f) C₃₋₉ cycloalkyl,
(g) a 5- to 12-membered aromatic heterocyclic group optionally having 1 to 3 substituents selected from
   (i) a halogen atom,
   (ii) hydroxy,
   (iii) C₁₋₆ alkyl optionally having 1 to 3 halogen atoms, and
   (iv) C₁₋₆ alkoxy optionally having 1 to 3 halogen atoms, and
(h) a 4- to 12-membered non-aromatic heterocyclic group optionally having 1 to 3 substituents selected from
   (i) a halogen atom,
   (ii) hydroxy,
   (iii) C₁₋₆ alkyl optionally having 1 to 3 halogen atoms,
   (iv) C₁₋₆ alkoxy optionally having 1 to 3 halogen atoms, and
   (v) oxo;
      (20) C₂₋₆ alkenyloxy (e.g., ethenyloxy) optionally having 1 to 3 halogen atoms;
      (21) C₃₋₈ cycloalkyloxy (e.g., cyclopropoxy, cyclopentyloxy) optionally having 1 to 3 substituents selected from
(a) a halogen atom, and
(b) C₁₋₆ alkoxy;
   (22) C₆₋₁₀ aryloxy (e.g., phenyloxy, naphthyloxy);
   (23) C₇₋₁₃ aralkyloxy (e.g., benzyloxy);
   (24) C₁₋₆ alkyl-carbonyloxy (e.g., acetyloxy, tert-butylcarbonyloxy);
   (25) C₆₋₁₀ aryl-carbonyl (e.g., benzoyl, 1-naphthoyl, 2-naphthoyl) optionally having 1 to 3 substituents selected from
(a) a halogen atom,
(b) C₁₋₆ alkyl optionally having 1 to 3 halogen atoms,
(c) C₁₋₆ alkoxy, and
(d) C₆₋₁₀ aryl;
   (26) 5- to 12-membered (preferably 5- to 7-membered) aromatic heterocyclyl-carbonyl (e.g., thienylcarbonyl, pyrazolylcarbonyl, pyrazinylcarbonyl, isoxazolylcarbonyl, pyridylcarbonyl, thiazolylcarbonyl, furylcarbonyl, pyrimidinylcarbonyl, benzothienylcarbonyl) optionally having 1 to 3 substituents selected from
(a) a halogen atom,
(b) C₁₋₆ alkyl optionally having 1 to 3 halogen atoms, and
(c) cyano;
   (27) 4- to 12-membered (preferably 4- to 7-membered) non-aromatic heterocyclyl-carbonyl (e.g., pyrrolidinylcarbonyl, morpholinylcarbonyl) optionally having 1 to 3 substituents selected from
(a) a halogen atom, and
(b) C₁₋₆ alkyl optionally having 1 to 3 halogen atoms;
   (28) C₇₋₁₃ aralkyloxy-carbonyl (e.g., benzyloxycarbonyl);
   (29) mercapto;
   (30) C₁₋₆ alkylthio (e.g., methylthio, ethylthio) optionally having 1 to 3 substituents selected from
(a) a halogen atom, and
(b) C₁₋₆ alkoxy-carbonyl;
   (31) C₇₋₁₃ aralkylthio (e.g., benzylthio);
   (32) C₆₋₁₀ arylthio (e.g., phenylthio, naphthylthio);
   (33) C₁₋₃ alkyleneoxy (e.g., methyleneoxy, ethyleneoxy);
   (34) C₁₋₃ alkylenedioxy (e.g., methylenedioxy, ethylenedioxy); and
   (35) C₂₋₆ alkenyl-carbonyl (e.g., vinylcarbonyl, allylcarbonyl) optionally having one C₆₋₁₀ aryl.

The “nitrogen-containing heterocycle” of the “nitrogen-containing heterocycle optionally having substituent(s)” in the present specification means a nitrogen-containing aromatic heterocycle or a nitrogen-containing non-aromatic heterocycle, each containing at least one nitrogen atom as a ring-constituting atom.

Examples of the nitrogen-containing aromatic heterocycle include a 5- to 7-membered (preferably 5- or 6-membered) monocyclic nitrogen-containing aromatic heterocycle and a 8- to 12-membered fused nitrogen-containing aromatic heterocycle.

Specific examples of the 5- to 7-membered (preferably 5- or 6-membered) monocyclic nitrogen-containing aromatic heterocycle include pyridine, pyrimidine, pyridazine, pyrazine, pyrrole, imidazole, pyrazole, thiazole, isothiazole, oxazole, isoxazole, oxadiazole, thiadiazole, triazole (e.g., 1,2,3-triazole, 1,2,4-triazole), tetrazole and triazine.

Specific examples of the 8- to 12-membered fused nitrogen-containing aromatic heterocycle include quinoline, isoquinoline, quinazoline, quinoxaline, benzoxazole, benzisoxazole, benzothiazole, benzimidazole (e.g., 1H-benzimidazole), benzotriazole (e.g., 1H-1,2,3-benzotriazole), indole, indazole, pyrrolopyrazine, imidazopyridine (e.g., 1H-imidazo[4,5-b]pyridine, 1H-imidazo[4,5-c]pyridine), thienopyridine, imidazopyrazine, pyrazolopyridine, pyrazolothiophene and pyrazolotriazine.

Examples of the nitrogen-containing non-aromatic heterocycle include a 4- to 7-membered (preferably 5- or 6-membered) monocyclic nitrogen-containing non-aromatic heterocycle or a 6- to 12-membered fused nitrogen-containing non-aromatic heterocycle.

Specific examples of the 4- to 7-membered (preferably 5- or 6-membered) monocyclic nitrogen-containing non-aromatic heterocycle include azetidine, pyrrolidine, piperidine, morpholine, piperazine, oxazolidine, thiazolidine, imidazolidine, oxazoline, thiazoline, imidazoline, pyrazolidine, pyrazoline, dihydropyridine (e.g., 1,2-dihydropyridine), tetrahydropyridine (e.g., 1,2,3,4-tetrahydropyridine, 1,2,5,6-tetrahydropyridine), dihydropyrimidine (e.g., 1,2-dihydropyrimidine) and tetrahydropyrimidine (e.g., 1,2,5,6-tetrahydropyrimidine).

Specific examples of the 6- to 12-membered fused nitrogen-containing non-aromatic heterocycle include dihydroindole (e.g., indoline), dihydroisoindole (e.g., isoindoline), dihydroquinoline (e.g., 1,2-dihydroquinoline), tetrahydroquinoline (e.g., 1,2,3,4-tetrahydroquinoline), dihydroisoquinoline (e.g., 1,2-dihydroisoquinoline), tetrahydroisoquinoline (e.g., 1,2,3,4-tetrahydroisoquinoline), dihydrophthalazine (e.g., 1,2-dihydrophthalazine) and azabicyclohexane (e.g., 2-azabicyclo[3.1.0]hexane).

When the “nitrogen-containing heterocycle optionally having substituent(s)” is a “nitrogen-containing aromatic heterocycle optionally having substituent(s)”, examples of the substituents that the nitrogen-containing aromatic heterocycle optionally has include substituents selected from the following Substituent Group B. The number of the substituents is not particularly limited as long as it is a substitutable, number, which is preferably 1 to 5, more preferably 1 to 3. When plural substituents are present, they may be the same or different.

### Substituent Group B:

(1) a substituent selected from the above-mentioned Substituent Group A;
(2) C₁₋₆ alkyl optionally having 1 to 3 substituents selected from
(a) a halogen atom,
(b) hydroxy,
(c) carboxy,
(d) C₁₋₆ alkoxy,
(e) C₁₋₆ alkoxy-carbonyl, and
(f) amino optionally having 1 or 2 C₁₋₆ alkyl;
   (3) C₂₋₆ alkenyl optionally having 1 to 3 substituents selected from
(a) a halogen atom,
(b) hydroxy,
(c) carboxy,
(d) C₁₋₆ alkoxy,
(e) C₁₋₆ alkoxy-carbonyl, and
(f) amino optionally having 1 or 2 C₁₋₆ alkyl; and
   (4) C₇₋₁₃ aralkyl optionally having 1 to 3 substituents selected from
(a) a halogen atom,
(b) hydroxy,
(c) C₁₋₆ alkyl optionally having 1 to 3 halogen atoms, and
(d) C₁₋₆ alkoxy.

When the “nitrogen-containing heterocycle optionally having substituent(s)” is a “nitrogen-containing non-aromatic heterocycle optionally having substituent(s)”, examples of the substituents that the nitrogen-containing non-aromatic heterocycle optionally has include substituents selected from the following Substituent Group C. The number of the substituents is not particularly limited as long as it is a substitutable number, which is preferably 1 to 5, more preferably 1 to 3. When plural substituents are present, they may be the same or different.

### Substituent Group C:

(1) a substituent selected from the above-mentioned Substituent Group A;
(2) C₁₋₆ alkyl optionally having 1 to 3 substituents selected from
(a) a halogen atom,
(b) hydroxy,
(c) carboxy,
(d) C₁₋₆ alkoxy,
(e) C₁₋₆ alkoxy-carbonyl, and
(f) amino optionally having 1 or 2 C₁₋₆ alkyl;
   (3) C₂₋₆ alkenyl optionally having 1 to 3 substituents selected from
(a) a halogen atom,
(b) hydroxy,
(c) carboxy,
(d) C₁₋₆ alkoxy,
(e) C₁₋₆ alkoxy-carbonyl, and
(f) amino optionally having 1 or 2 C₁₋₆ alkyl;
   (4) C₇₋₁₃ aralkyl optionally having 1 to 3 substituents selected from
(a) a halogen atom,
(b) hydroxy,
(c) C₁₋₆ alkyl optionally having 1 to 3 halogen atoms, and
(d) C₁₋₈ alkoxy; and
   (5) oxo.

In the specification, examples of the substituents that “4- to 7-membered nitrogen-containing non-aromatic heterocycle” of the “4- to 7-membered nitrogen-containing non-aromatic heterocycle optionally having substituent(s)” optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

The “4- to 7-membered nitrogen-containing non-aromatic heterocycle” in the present specification means, for example, the 4- to 7-membered monocyclic nitrogen-containing non-aromatic heterocycle” exemplified as the above-mentioned “nitrogen-containing heterocycle”.

The “cyclic group” of the “cyclic group optionally having substituent(s)” in the present specification means, for example, C₃₋₈ cycloalkyl, a monovalent fused ring group derived from a fused ring formed by condensation of C₃₋₈ cycloalkane and a benzene ring (e.g., indanyl, 1,2,3,4-tetrahydronaphthyl), C₆₋₁₀ aryl, an aromatic heterocyclic group, a non-aromatic heterocyclic group or the like.

When the “cyclic group optionally having substituent(s)” is C₃₋₈ cycloalkyl optionally having substituent(s), examples of the substituents that the C₃₋₈ cycloalkyl optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

When the “cyclic group optionally having substituent(s)” is a monovalent fused ring group derived from a fused ring formed by condensation of C₃₋₈ cycloalkane and a benzene ring, which optionally has substituent(s), examples of the substituents that the fused ring group optionally has include substituents selected from the above-mentioned Substituent Group C. The position of the substituent is not particularly limited as long as it is a substitutable position, and may be a benzene ring part or a C₃₋₉ cycloalkane part. The number of the substituents is not particularly limited as long as it is a substitutable number, which is preferably 1 to 5, more preferably 1 to 3. When plural substituents are present, they may be the same or different.

When the “cyclic group optionally having substituent(s)” is C₆₋₁₀ aryl optionally having substituent(s), examples of the substituents that the C₆₋₁₀ aryl optionally has include substituents selected from the above-mentioned Substituent Group B. The number of the substituents is not particularly limited as long as it is a substitutable number, which is preferably 1 to 5, more preferably 1 to 3. When plural substituents are present, they may be the same or different.

When the “cyclic group optionally having substituent(s)” is an aromatic heterocyclic group optionally having substituent(s), examples of the substituents that the aromatic heterocyclic group optionally has include substituents selected from the above-mentioned Substituent Group B. The number of the substituents is not particularly limited as long as it is a substitutable number, which is preferably 1 to 5, more preferably 1 to 3. When plural substituents are present, they may be the same or different.

When the “cyclic group optionally having substituent(s)” is a non-aromatic heterocyclic group optionally having substituent(s), examples of the substituents that the non-aromatic heterocyclic group optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

The “divalent chain aliphatic hydrocarbon group optionally having substituent(s)” in the present specification means, for example, C₁₋₆ alkylene optionally having substituent(s), C₂₋₆ alkenylene optionally having substituent(s), C₂₋₆ alkynylene optionally having substituent(s) or the like.

The “divalent cyclic group optionally having substituent(s)” in the present specification means, for example, C₃₋₈ cycloalkylene optionally having substituent(s), a divalent group derived from a fused ring formed by condensation of C₃₋₆ cycloalkane and a benzene ring, which optionally has substituent(s), C₆₋₁₀ arylene optionally having substituent(s), a divalent aromatic heterocyclic group optionally having substituent(s), a divalent non-aromatic heterocyclic group optionally having substituent(s) or the like.

The “C₁₋₆ alkylene” in the present specification means, for example, —CH₂—, —(CH₂)₂—, —(CH₂)₃—, —(CH₂)₄—, —(CH₂)₅—, —(CH₂)₆—, —CH(CH₃)—C(CH₃)₂—, —CH(C₂H₅)—CH(C₃H₇)—, (CH(CH₃)₂)—, —(CH(CH₃))₂—, —CH₂—CH(CH₃)—, (CH₃)— (CH₂)₂—C(CH₃)₂—, —C(CH₃)₂—(CH₂)₂—, (CH₂)₃—C(CH₃)₂—, —C(CH₃)₂—(CH₂)₃— or the like.

In the present specification, examples of the substituent that the “C₁₋₆ alkylene” of the “C₁₋₆ alkylene optionally having substituent(s)” optionally has include substituents selected from the above-mentioned Substituent Group A. The number of the substituents is not particularly limited as long as it is a substitutable number, which is preferably 1 to 5, more preferably 1 to 3. When plural substituents are present, they may be the same or different.

The “C₂₋₆ alkenylene” in the present specification means, for example, —CH═CH—, —CH₂—CH═CH—, —CH═CH—CH₂—, —C(CH₃)₂—CH═CH—, —CH═CH—C(CH₃)₂—, —CH₂—CH═CH—CH₂—, —(CH₂)₂—CH═CH—, —CH═CH—(CH₂)₂—, —CH═CH—CH═CH—, —CH═CH—(CH₂)₃—, —(CH₂)₃—CH═CH— or the like.

In the present specification, examples of the substituent that the “C₂₋₆ alkenylene group” of the “C₂₋₆ alkenylene optionally having substituent(s)” optionally has include substituents selected from the above-mentioned Substituent Group A. The number of the substituents is not particularly limited as long as it is a substitutable number, which is preferably 1 to 5, more preferably 1 to 3. When plural substituents are present, they may be the same or different.

The “C₂₋₆ alkynylene” in the present specification means, for example, —C≡C—, —C₂—C≡C—, —C≡C—CH₂—, —C(CH₃)₂—C≡C—, —C≡C—C(CH₃)₂—, —CH₂—C≡C—CH₂—, —(CH₂)₂—C≡C—, —C≡C—(CH₂)₂—, —C≡C—C≡C—, —C≡C—(CH₂)₃—, —(CH₂)₃—C≡C— or the like.

In the present specification, examples of the substituent that the “C₂₋₆ alkynylene” of the “C₂₋₆ alkynylene optionally having substituent(s)” optionally has include substituents selected from the above-mentioned Substituent Group A. The number of the substituents is not particularly limited as long as it is a substitutable number, which is preferably 1 to 5, more preferably 1 to 3. When plural substituents are present, they may be the same or different.

The “C₃₋₆ cycloalkylene” in the present specification means, for example, cyclopropylene, cyclobutylene (e.g., 1,2-cyclobutylene, 1,3-cyclobutylene), cyclopentylene (e.g., 1,2-cyclopentylene, 1,3-cyclopentylene), cyclohexylene (e.g., 1,2-cyclohexylene, 1,3-cyclohexylene, 1,4-cyclohexylene) or the like.

In the present specification, examples of the substituent that the “C₃₋₆ cycloalkylene” of the “C₃₋₆ cycloalkylene optionally having substituent(s)” optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

The “C₆₋₁₀ arylene” in the present specification means, for example, phenylene (e.g., 1,2-phenylene, 1,3-phenylene, 1,4-phenylene), naphthylene (e.g., 1,2-naphthylene, 1,3-naphthylene, 1,4-naphthylene, 1,5-naphthylene, 1,6-naphthylene, 1,7-naphthylene, 1,8-naphthylene, 2,3-naphthylene, 2,4-naphthylene, 2,5-naphthylene, 2,6-naphthylene, 2,7-naphthylene, 2,8-naphthylene) or the like.

In the present specification, examples of the substituent that the “C₆₋₁₀ arylene” of the “C₆₋₁₀ arylene optionally having substituent(s)” optionally has include substituents selected from the above-mentioned Substituent Group B. The number of the substituents is not particularly limited as long as it is a substitutable number, which is preferably 1 to 5, more preferably 1 to 3. When plural substituents are present, they may be the same or different.

The “divalent group derived from a fused ring formed by condensation of C₃₋₆ cycloalkane and a benzene ring” in the present specification means, for example, indane-diyl (e.g., indane-1,2-diyl, indane-1,3-diyl), tetrahydronaphthalene-diyl (e.g., 1,2,3,4-tetrahydronaphthalene-1,2-diyl, 1,2,3,4-tetrahydronaphthalene-1,3-diyl, 1,2,3,4-tetrahydronaphthalene-1,4-diyl, 1,2,3,4-tetrahydronaphthalene-2,3-diyl) or the like.

In the present specification, examples of the substituent that the “divalent group derived from a fused ring formed by condensation of C₃₋₆ cycloalkane and a benzene ring” of the “divalent group derived from a fused ring formed by condensation of C₃₋₆ cycloalkane and a benzene ring, which optionally has substituent(s)” optionally has include substituents selected from the above-mentioned Substituent Group C. The position of the substituent is not particularly limited as long as it is a substitutable position, and may be a benzene ring part or a C₃₋₈ cycloalkane part. The number of the substituents is not particularly limited as long as it is a substitutable number, which is preferably 1 to 5, more preferably 1 to 3. When plural substituents are present, they may be the same or different.

The “divalent aromatic heterocyclic group” in the present specification means a divalent group derived from the above-mentioned aromatic heterocyclic group, and specific examples thereof include 5- to 7-membered (preferably 5- or 6-membered) divalent monocyclic aromatic heterocyclic groups such as furan-diyl, thiophene-diyl, pyridine-diyl, pyrimidine-diyl, pyridazine-diyl, pyrazine-diyl, pyrrole-diyl, imidazole-diyl, pyrazole-diyl, thiazole-diyl, isothiazole-diyl, oxazole-diyl, isoxazole-diyl, oxadiazole-diyl, triazole-diyl; and 8- to 12-membered divalent fused aromatic heterocyclic groups such as quinoline-diyl, isoquinoline-diyl, quinazoline-diyl, quinoxaline-diyl, benzofuran-diyl, benzothiophene-diyl, benzoxazole-diyl, benzisoxazole-diyl, benzothiazole, benzoisothiazole, benzimidazole-diyl, benzotriazole-diyl, indole-diyl, indazole-diyl, pyrrolopyrazine-diyl, imidazopyridine-diyl, thienopyridine-diyl, imidazopyrazine-diyl, pyrazolopyridine-diyl, pyrazolothiophene-diyl; pyrazolotriazine-diyl.

In the present specification, examples of the substituent that the “divalent aromatic heterocyclic group” of the “divalent aromatic heterocyclic group optionally having substituent(s)” optionally has include substituents selected from the above-mentioned Substituent Group B. The number of the substituents is not particularly limited as long as it is a substitutable number, which is preferably 1 to 5, more preferably 1 to 3. When plural substituents are present, they may be the same or different.

The “divalent non-aromatic heterocyclic group” in the present specification means a divalent group derived from the so above-mentioned non-aromatic heterocyclic group, and specific examples thereof include 4- to 7-membered (preferably 5- or 6-membered) monocyclic non-aromatic heterocyclic groups such as azetidine-diyl, pyrrolidine-diyl, piperidine-diyl, morpholine-diyl, thiomorpholine-diyl, piperazine-diyl, oxazolidine-diyl, thiazolidine-diyl, imidazolidine-diyl, oxazoline-diyl, thiazoline-diyl, imidazoline-diyl, dioxole-diyl, dioxolan-diyl, dihydrooxadiazole-diyl, pyran-diyl, tetrahydropyran-diyl, thiopyran-diyl, dihydrothiopyran-diyl, tetrahydrothiopyran-diyl, 1-oxidotetrahydrothiopyran-diyl, 1,1-dioxidotetrahydrothiopyran-diyl, tetrahydrofuran-diyl, pyrazolidine-diyl, pyrazoline-diyl, tetrahydropyrimidine-diyl, dihydrotriazole-diyl, tetrahydrotriazole-diyl, azepane-diyl, dihydropyridine-diyl, tetrahydropyridine-diyl; and 8- to 12-membered fused non-aromatic heterocyclic groups such as dihydroindole-diyl, dihydroisoindole-diyl, dihydrobenzofuran-diyl, tetrahydrobenzofuran-diyl, dihydrobenzodioxine-diyl, dihydrobenzodioxepine-diyl, chromene-diyl, dihydrochromene-diyl, dihydroquinoline-diyl, tetrahydroquinoline-diyl, dihydroisoquinoline-diyl, tetrahydroisoquinoline-diyl, dihydrophthalazine-diyl, azabicyclohexane-diyl.

In the present specification, examples of the substituent that the “divalent non-aromatic heterocyclic group” of the “divalent non-aromatic heterocyclic group optionally having substituent(s)” optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

Compound (I) is explained in the following.

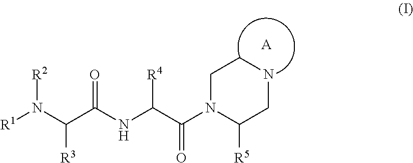

In the formula (I), the moiety represented by the formula:

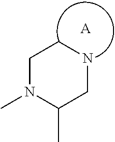

is a divalent group derived from a bicyclic ring formed by condensation of piperazine and ring A.

As used herein, ring A is a nitrogen-containing heterocycle optionally having substituent(s).

Preferable examples of the “nitrogen-containing heterocycle” of the “nitrogen-containing heterocycle optionally having substituent(s)” for ring A include the following:

(1) a 5- to 7-membered (preferably 5- or 6-membered) monocyclic nitrogen-containing aromatic heterocycle having, as a ring-constituting member, “—CH₂—NH—” or “═CH—NH—” capable of forming a covalent linkage with piperazine (e.g., pyrrole, imidazole, pyrazole, triazole (e.g., 1,2,3-triazole, 1,2,4-triazole), tetrazole);
(2) a 8- to 12-membered fused nitrogen-containing aromatic heterocycle having, as a ring-constituting member, “—CH₂—NH—” or “═CH—NH—” capable of forming a covalent linkage with piperazine (e.g., benzimidazole (e.g., 1H-benzimidazole), benzotriazole (e.g., 1H-1,2,3-benzotriazole), indole, imidazopyridine (e.g., 1H-imidazo[4,5-b]pyridine, 1H-imidazo[4,5-c]pyridine));
(3) a 4- to 7-membered (preferably 5- or 6-membered) monocyclic nitrogen-containing non-aromatic heterocycle having, as a ring-constituting member, “—CH₂—NH—” or “═CH—NH—” capable of forming a covalent linkage with piperazine (e.g., azetidine, pyrrolidine, imidazolidine, pyrazolidine, piperidine, piperazine, morpholine); and
(4) a 6- to 12-membered fused nitrogen-containing non-aromatic heterocycle having, as a ring-constituting member, “—CH₂—NH—” or “═CH—NH—” capable of forming a covalent linkage with piperazine (e.g., dihydroindole (e.g., indoline), dihydroisoindole (e.g., isoindoline), dihydroquinoline (e.g., 1,2-dihydroquinoline), tetrahydroquinoline (e.g., 1,2,3,4-tetrahydroquinoline), dihydroisoquinoline (e.g., 1,2-dihydroisoquinoline), tetrahydroisoquinoline (e.g., 1,2,3,4-tetrahydroisoquinoline), azabicyclohexane (e.g., 2-azabicyclo[3.1.0]hexane)).

The “nitrogen-containing heterocycle” of the “nitrogen-containing heterocycle optionally having substituent(s)” for ring A is preferably a 4- to 7-membered (preferably 5- or 6-membered) monocyclic nitrogen-containing non-aromatic heterocycle or a 6- to 12-membered fused nitrogen-containing non-aromatic heterocycle, each having “—CH₂—NH—” or “═CH—NH—” as a ring-constituting member, more preferably pyrrolidine, dihydroindole (e.g., indoline) or azabicyclohexane (e.g., 2-azabicyclo[3.1.0]hexane), particularly preferably pyrrolidine.

When the “nitrogen-containing heterocycle” of the “nitrogen-containing heterocycle optionally having substituent(s)” for ring A is a 5- to 7-membered monocyclic nitrogen-containing aromatic heterocycle or a 8- to 12-membered fused nitrogen-containing aromatic heterocycle, each having “—CH₂—NH—” or “═CH—NH—” as a ring-constituting member, preferable specific examples of the substituents that the ring optionally has include the following:

(1) a halogen atom;
(2) hydroxy;
(3) C₁₋₆ alkyl optionally having 1 to 3 halogen atoms;
(4) C₁₋₆ alkoxy optionally having 1 to 3 substituents selected from a halogen atom, C₁₋₆ alkoxy and C₃₋₈ cycloalkyl (e.g., cyclopropyl);
(5) C₃₋₈ cycloalkyloxy (e.g., cyclopropoxy, cyclopentyloxy);
(6) C₆₋₁₀ aryloxy (e.g., phenyloxy, naphthyloxy); and
(7) C₇₋₁₃ aralkyloxy (e.g., benzyloxy).

The number of the substituents is not particularly limited as long as it is a substitutable number, which is preferably 1 to 5, more preferably 1 to 3. When plural substituents are present, they may be the same or different.

When the “nitrogen-containing heterocycle” of the “nitrogen-containing heterocycle optionally having substituent(s)” for ring A is a 4- to 7-membered monocyclic nitrogen-containing non-aromatic heterocycle or a 6- to 12-membered fused nitrogen-containing non-aromatic heterocycle, each having “—CH₂—NH—” or “═CH—NH—” as a ring-constituting member, preferable specific examples of the substituents that the ring optionally has include the following:

(1) a halogen atom;
(2) hydroxy;
(3) C₁₋₆ alkyl optionally having 1 to 3 halogen atoms;
(4) C₁₋₆ alkoxy optionally having 1 to 3 substituents selected from a halogen atom, C₁₋₆ alkoxy and C₃₋₈ cycloalkyl (e.g., cyclopropyl);
(5) C₃₋₈ cycloalkyloxy (e.g., cyclopropoxy, cyclopentyloxy);
(6) C₆₋₁₀ aryloxy (e.g., phenyloxy, naphthyloxy);
(7) C₇₋₁₃ aralkyloxy (e.g., benzyloxy); and
(8) oxo.

The number of the substituents is not particularly limited as long as it is a substitutable number, which is preferably 1 to 5, more preferably 1 to 3. When plural substituents are present, they may be the same or different.

When the “nitrogen-containing heterocycle” of the “nitrogen-containing heterocycle optionally having substituent(s)” for ring A is a 4- to 7-membered monocyclic nitrogen-containing non-aromatic heterocycle having “—CH₂—NH—” or “═CH—NH—” as a ring-constituting member, more preferable specific examples of the substituents that the ring optionally has include a halogen atom and C₁₋₆ alkoxy optionally having 1 to 3 halogen atoms. Of these, C₁₋₈ alkoxy optionally having 1 to 3 halogen atoms is preferable, and C₁₋₆ alkoxy is particularly preferable.

Specific examples of ring A include the following:

(1) a 5- to 7-membered (preferably 5- or 6-membered) monocyclic nitrogen-containing aromatic heterocycle having “—CH₂—NH—” or “═CH—NH—” as a ring-constituting member (e.g., pyrrole, imidazole, pyrazole, triazole (e.g., 1,2,3-triazole, 1,2,4-triazole), tetrazole) and optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy); and
(g) C₇₋₁₃ aralkyloxy (e.g., benzyloxy);
   (2) a 8- to 12-membered fused nitrogen-containing aromatic heterocycle having “—CH₂—NH—” or “═CH—NH—” as a ring-constituting member (e.g., benzimidazole (e.g., 1H-benzimidazole), benzotriazole (e.g., 1H-1,2,3-benzotriazole), indole, imidazopyridine (e.g., 1H-imidazo[4,5-b]pyridine, 1H-imidazo[4,5-c]pyridine)) and optionally having 1 to 5 (preferably 1 to 3) substituents selected from
(a) a halogen atom;
(b) hydroxy;
(c) C₁₋₉ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy); and
(g) C₇₋₁₃ aralkyloxy (e.g., benzyloxy);
   (3) a 4- to 7-membered (preferably 5- or 6-membered) monocyclic nitrogen-containing non-aromatic heterocycle having “—CH₂—NH—” or “═CH—NH—” as a ring-constituting member (e.g., azetidine, pyrrolidine, imidazolidine, pyrazolidine, piperidine, piperazine, morpholine) and optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₉ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy);
(g) C₇₋₁₃ aralkyloxy (e.g., benzyloxy); and
(h) oxo; and
   (4) a 6- to 12-membered fused nitrogen-containing non-aromatic heterocycle having “—CH₂—NH—” or “═CH—NH—” as a ring-constituting member (e.g., dihydroindole (e.g., indoline), dihydroisoindole (e.g., isoindoline), dihydroquinoline (e.g., 1,2-dihydroquinoline), tetrahydroquinoline (e.g., 1,2,3,4-tetrahydroquinoline), dihydroisoquinoline (e.g., 1,2-dihydroisoquinoline), tetrahydroisoquinoline (e.g., 1,2,3,4-tetrahydroisoquinoline), azabicyclohexane (e.g., 2-azabicyclo[3.1.0]hexane)) and optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy);
(g) C₇₋₁₃ aralkyloxy (e.g., benzyloxy); and
(h) oxo.

Ring A is preferably pyrrolidine, dihydroindole (e.g., indoline) or azabicyclohexane (e.g., 2-azabicyclo[3.1.0]hexane), each optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy);
(g) C₇₋₁₃ aralkyloxy (e.g., benzyloxy); and
(h) oxo.

As another embodiment, ring A is preferably a 4- to 7-membered (preferably 5- or 6-membered) monocyclic nitrogen-containing non-aromatic heterocycle (e.g., azetidine, pyrrolidine, imidazolidine, pyrazolidine, piperidine, piperazine, morpholine) optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy);
(g) C₇₋₁₃ aralkyloxy (e.g., benzyloxy); and
(h) oxo.

As another embodiment, ring A is preferably

(1) a 4- to 7-membered (preferably 5- or 6-membered) monocyclic nitrogen-containing non-aromatic heterocycle (e.g., pyrrolidine) optionally having 1 to 5 (preferably 1 to 3) substituents selected from
(a) a halogen atom (e.g., a fluorine atom);
(b) hydroxy;
(c) C₁₋₆ alkyl (e.g., methyl) optionally having 1 to 3 halogen atoms (e.g., a fluorine atom);
(d) C₁₋₆ alkoxy (e.g., methoxy, ethoxy) optionally having 1 to 3 substituents selected from
   (i) a halogen atom (e.g., a fluorine atom),
   (ii) C₁₋₆ alkoxy (e.g., ethoxy), and
   (iii) C₃₋₈ cycloalkyl (e.g., cyclopropyl); and
(e) oxo; or
   (2) a 6- to 12-membered fused nitrogen-containing non-aromatic heterocycle having “—C₁₋₁₂—NH—” as a ring-constituting member (e.g., azabicyclohexane (e.g., 2-azabicyclo[3.1.0]hexane)), more preferably
   (1) a 4- to 7-membered (preferably 5- or 6-membered) monocyclic nitrogen-containing non-aromatic heterocycle (e.g., pyrrolidine) optionally having 1 to 5 (preferably 1 to 3) substituents selected from
(a) a halogen atom (e.g., a fluorine atom);
(b) hydroxy;
(c) C₁₋₆ alkyl (e.g., methyl) optionally having 1 to 3 halogen atoms (e.g., a fluorine atom);
(d) C₁₋₆ alkoxy (e.g., methoxy, ethoxy) optionally having 1 to 3 substituents selected from
   (i) a halogen atom (e.g., a fluorine atom),
   (ii) C₁₋₆ alkoxy (e.g., ethoxy), and
   (iii) C₃₋₈ cycloalkyl (e.g., cyclopropyl); and
(e) oxo.

Ring A is more preferably pyrrolidine optionally having 1 to 5 (preferably 1 to 3) substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl (e.g., cyclopropyl);
(e) C₃₋₈ cycloalkyloxy (e.g., cyclopropoxy, cyclopentyloxy);
(f) C₆₋₁₀ aryloxy (e.g., phenyloxy, naphthyloxy);
(g) C₇₋₁₃ aralkyloxy (e.g., benzyloxy); and
(h) oxo.

Ring A is still more preferably pyrrolidine optionally having 1 to 3 substituents selected from C₁₋₆ alkoxy (e.g., ethoxy) optionally having 1 to 3 halogen atoms and a halogen atom, particularly preferably pyrrolidine optionally having C₁₋₆ alkoxy (e.g., ethoxy) optionally having 1 to 3 halogen atoms, most preferably pyrrolidine optionally having C₁₋₆ alkoxy (e.g., ethoxy).

A compound wherein ring A is pyrrolidine shows a high cIAP antagonistic activity, and the high cIAP antagonistic activity as compared to a XIAP antagonistic activity. In addition, a compound wherein ring A is pyrrolidine affords a strong pharmacological activity.

A compound wherein ring A is pyrrolidine having C₁₋₆ alkoxy (e.g., ethoxy) is superior in metabolic stability. In addition, a compound wherein ring A is pyrrolidine having C₁₋₆ alkoxy (e.g., ethoxy) is superior in pharmacokinetics. Moreover, a compound wherein ring A is pyrrolidine having C₁₋₆ alkoxy (e.g., ethoxy) is superior since a crystalline compound can be obtained easily.

To provide a compound superior in the XIAP antagonistic activity, when ring A is pyrrolidine having C₁₋₆ alkoxy (e.g., ethoxy), the absolute configuration of the carbon atom on the pyrrolidine that the C₁₋₆ alkoxy (e.g., ethoxy) is bonded to is preferably R-configuration.

As another embodiment, ring A is preferably a nitrogen-containing heterocycle having C₁₋₆ alkoxy.

As another embodiment, ring A is preferably an unsubstituted nitrogen-containing heterocycle.

As another embodiment, ring A is preferably a 4- to 7-membered monocyclic nitrogen-containing non-aromatic heterocycle optionally having substituent(s), or a 6- to 12-membered fused nitrogen-containing non-aromatic heterocycle optionally having substituent(s).

In the formula (I), R¹ is a hydrogen atom, a chain aliphatic hydrocarbon group optionally having substituent(s), —OR¹¹, —C(═O)—R¹¹, —C(═O)—O—R¹¹, —N(R¹²)—R¹¹ or —C(═O)—N(R¹²)—R¹¹. As used herein, R¹¹ is a chain aliphatic hydrocarbon group optionally having substituent(s), and R¹² is a hydrogen atom or a chain aliphatic hydrocarbon group optionally having substituent(s).

R¹ is preferably C₁₋₆ alkyl (e.g., methyl) optionally having substituent(s).

As another embodiment, R¹ is preferably a hydrogen atom, C₁₋₆ alkyl (e.g., methyl) optionally having substituent(s) or —OR¹¹ wherein R¹¹ is as defined above.

R¹ is more preferably

(1) a hydrogen atom;
(2) C₁₋₆ alkyl (e.g., methyl, ethyl) optionally having 1 to 3 substituents selected from
(a) amino,
(b) imino, and
(c) C₃₋₈ cycloalkyl (e.g., cyclopropyl); or
   (3) hydroxy.

R¹ is still more preferably

(1) a hydrogen atom; or
(2) C₁₋₆ alkyl (e.g., methyl, ethyl) optionally having 1 to 3 substituents selected from
(a) amino,
(b) imino, and
(c) C₃₋₈ cycloalkyl (e.g., cyclopropyl).

R¹ is particularly preferably C₁₋₈ alkyl (e.g., methyl).

In the formula (I), R² is a hydrogen atom, a chain aliphatic hydrocarbon group optionally having substituent(s), —C(═O)—R²¹, —C(═O)—O—R²¹ or —C(═O)—N(R²²)—R²¹. As used herein, R²¹ is a chain aliphatic hydrocarbon group optionally having substituent(s), and R²² is a hydrogen atom or a chain aliphatic hydrocarbon group optionally having substituent(s).

R² is preferably a hydrogen atom or C₁₋₆ alkyl (e.g., methyl) optionally having substituent(s).

R² is more preferably a hydrogen atom or C₁₋₆ alkyl (e.g., methyl).

R² is particularly preferably a hydrogen atom.

In the formula (I), R³ is a hydrogen atom or a chain aliphatic hydrocarbon group optionally having substituent(s).

R³ is preferably a hydrogen atom, C₁₋₆ alkyl (e.g., methyl) optionally having substituent(s), or C₃₋₈ cycloalkyl (e.g., cyclopropyl) optionally having substituent(s).

Preferable specific examples of R³ include

(1) a hydrogen atom;
(2) C₁₋₆ alkyl (e.g., methyl, ethyl, isopropyl) optionally having 1 to 3 substituents selected from
(a) a halogen atom (e.g., a fluorine atom),
(b) hydroxy,
(c) cyano, and
(d) C₃₋₈ cycloalkyl (e.g., cyclopropyl); and
   (3) C₃₋₈ cycloalkyl (e.g., cyclopropyl).

R³ is particularly preferably C₁₋₆ alkyl (e.g., methyl, ethyl, isopropyl).

As another embodiment, R¹ and R² in combination form, together with the adjacent nitrogen atom, a 4- to 7-membered nitrogen-containing non-aromatic heterocycle optionally having substituent(s).

As another embodiment, R¹ and R³ in combination form, together with the adjacent nitrogen atom and carbon atom, a 4- to 7-membered nitrogen-containing non-aromatic heterocycle optionally having substituent(s).

In the embodiment, preferably R¹ and R³ in combination form, together with the adjacent nitrogen atom and carbon atom, a 4- to 7-membered (preferably 4 to 6-membered) nitrogen-containing non-aromatic heterocycle (e.g., azetidine, pyrrolidine).

In the formula (I), R⁴ is a cyclic group optionally having substituent(s) or a chain aliphatic hydrocarbon group optionally having substituent(s).

R⁴ is preferably

(1) C₃₋₈ cycloalkyl (e.g., cyclohexyl) optionally having substituent(s);
(2) a 4- to 7-membered monocyclic non-aromatic heterocyclic group (e.g., tetrahydropyranyl, piperidyl, 1,1-dioxidotetrahydrothiopyranyl) optionally having substituent(s);
(3) C₁₋₆ alkyl (e.g., propyl, butyl) optionally having substituent(s); or
(4) C₆₋₁₀ aryl (e.g., phenyl) optionally having substituent(s).

R⁴ is more preferably

(1) C₃₋₈ cycloalkyl (e.g., cyclohexyl) optionally having 1 to 3 substituents selected from
(a) a halogen atom,
(b) hydroxy,
(c) C₁₋₆ alkoxy-carbonyl (e.g., methoxycarbonyl) optionally having one C₆₋₁₀ aryl (e.g., phenyl), and
(d) oxo;
   (2) a 4- to 7-membered monocyclic non-aromatic heterocyclic group (e.g., tetrahydropyranyl, piperidyl, 1,1-dioxidotetrahydrothiopyranyl);
   (3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl); or
   (4) C₆₋₁₀ aryl (e.g., phenyl, naphthyl) optionally having 1 to 3 halogen atoms (e.g., a fluorine atom).

R⁴ is further more preferably

(1) C₃₋₈ cycloalkyl (e.g., cyclohexyl) optionally having 1 to 3 halogen atoms (e.g., a fluorine atom);
(2) a 4- to 7-membered monocyclic non-aromatic heterocyclic group (e.g., tetrahydropyranyl);
(3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl); or
   (4) C₆₋₁₀ aryl (e.g., phenyl, naphthyl) optionally having 1 to 3 halogen atoms (e.g., a fluorine atom), still more preferably
   (1) C₃₋₈ cycloalkyl (e.g., cyclohexyl) optionally having 1 to 3 halogen atoms (e.g., a fluorine atom);
   (2) a 4- to 7-membered monocyclic non-aromatic heterocyclic group (e.g., tetrahydropyranyl); or
   (3) C₁₋₈ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl).

As another embodiment, R⁴ is preferably a cyclic group (preferably C₃₋₈ cycloalkyl, a 4- to 7-membered monocyclic non-aromatic heterocyclic group or C₆₋₁₀ aryl) having halogen atom(s).

As another embodiment, R⁴ is preferably unsubstituted cyclic group (preferably C₃₋₈ cycloalkyl, a 4- to 7-membered monocyclic non-aromatic heterocyclic group or C₆₋₁₀ aryl).

As another embodiment, R⁴ is more preferably C₃₋₈ cycloalkyl (e.g., cyclohexyl) optionally having 1 to 3 substituents selected from
(a) a halogen atom,
(b) hydroxy,
(c) C₁₋₆ alkoxy-carbonyl (e.g., methoxycarbonyl) optionally having one C₆₋₁₀ aryl (e.g., phenyl), and
(d) oxo.

A compound wherein R⁴ is C₃₋₈ cycloalkyl (e.g., cyclohexyl) having 1 to 3 halogen atoms (e.g., a fluorine atom) is superior since a crystalline compound can be easily obtained.

R⁵ is a cyclic group optionally having substituent(s), —C(═O)NR⁵¹—X¹—R⁵², —C(═S)NR⁵¹—X¹—R⁵², —X²—R⁵², —X²—NR⁵¹R⁵³, —X²—NR⁵¹—C(═O)—R⁵³ or —C(═NR⁵⁴)—NR⁵¹—X¹—R⁵². As used herein, X¹ is a bond or C₁₋₆ alkylene optionally having substituent(s), X² is C₁₋₆ alkylene optionally having substituent(s), R⁵¹ is a hydrogen atom, a chain aliphatic hydrocarbon group optionally having substituent(s) or a cyclic group optionally having substituent(s), R⁵² is a cyclic group optionally having substituent(s), R⁵³ is a chain aliphatic hydrocarbon group optionally having substituent(s) or a cyclic group optionally having substituent(s), and R⁵⁴ is a hydrogen atom or a chain aliphatic hydrocarbon group optionally having substituent(s).

R⁵ is preferably

(1) a 5- to 7-membered monocyclic aromatic heterocyclic group (e.g., thiazolyl, oxazolyl, pyrazolyl, oxadiazolyl) optionally having substituent(s);
(2) a 8- to 12-membered fused aromatic heterocyclic group (e.g., benzothiazolyl) optionally having substituent(s);
(3) —C(═O)NR⁵¹—X¹—R⁵² wherein R⁵¹, X¹ and R⁵² are defined above;
(4) —X²—R⁵² wherein X² and R⁵² are defined above; or
(5) —X²—NR⁵¹R⁵³ wherein R⁵¹, X² and R⁵³ are defined above.

Preferable specific examples of R⁵ include

(1) a 5- to 7-membered monocyclic aromatic heterocyclic group (e.g., thiazolyl, oxazolyl, pyrazolyl, oxadiazolyl) optionally having one substituent selected from
(a) C₆₋₁₀ aryl optionally having 1 to 3 halogen atoms,
(b) C₆₋₁₀ aryl-carbonyl (e.g., benzoyl, 1-naphthoyl) optionally having 1 to 3 substituents selected from
   (i) a halogen atom,
   (ii) C₁₋₆ alkyl optionally having 1 to 3 halogen atoms,
   (iii) C₁₋₆ alkoxy, and
   (iv) C₆₋₁₀ aryl,
(c) 5- to 7-membered monocyclic aromatic heterocyclyl-carbonyl (e.g., thienylcarbonyl, furylcarbonyl, pyrazolylcarbonyl, isooxazolylcarbonyl, pyrimidinylcarbonyl) optionally having 1 to 3 substituents selected from cyano and C₁₋₆ alkyl,
(d) 8- to 12-membered fused aromatic heterocyclyl-carbonyl (e.g., benzothienylcarbonyl),
(e) C₇₋₁₃ aralkyl optionally having 1 to 3 halogen atoms, and
(f) C₂₋₆ alkenyl-carbonyl (e.g., vinylcarbonyl, allylcarbonyl) optionally having one C₆₋₁₀ aryl;
   (2) a 8- to 12-membered fused aromatic heterocyclic group (e.g., benzothiazolyl) optionally having one substituent selected from
(a) C₆₋₁₀ aryl optionally having 1 to 3 halogen atoms,
(b) C₆₋₁₀ aryl-carbonyl (e.g., benzoyl, 1-naphthoyl) optionally having 1 to 3 substituents selected from
   (i) a halogen atom,
   (ii) C₁₋₆ alkyl optionally having 1 to 3 halogen atoms,
   (iii) C₁₋₆ alkoxy, and
   (iv) C₆₋₁₀ aryl,
(c) 5- to 7-membered monocyclic aromatic heterocyclyl-carbonyl (e.g., thienylcarbonyl, furylcarbonyl, pyrazolylcarbonyl, isooxazolylcarbonyl, pyrimidinylcarbonyl) optionally having 1 to 3 substituents selected from cyano and C₁₋₆ alkyl,
(d) 8- to 12-membered fused aromatic heterocyclyl-carbonyl (e.g., benzothienylcarbonyl),
(e) C₇₋₁₃ aralkyl optionally having 1 to 3 halogen atoms, and
(f) C₂₋₆ alkenyl-carbonyl (e.g., vinylcarbonyl, allylcarbonyl) optionally having one C₆₋₁₀ aryl;
   (3) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl, fluorenyl), which optionally has 1 to 3 substituents selected from
   (i) hydroxy,
   (ii) a halogen atom,
   (iii) C₁₋₆ alkyl-carbonyloxy (e.g., acetyloxy), and
   (iv) oxo;
(b) a monovalent group derived from a fused ring formed by condensation of tetrahydropyran or tetrahydrofuran and a benzene ring (e.g., 2,3-dihydrochromenyl, 2,3-dihydrobenzofuranyl), which optionally has 1 to 3 halogen atoms;
(c) C₆₋₁₀ aryl optionally having 1 to 3 C₁₋₈ alkyl
(d) a 5- to 7-membered monocyclic aromatic heterocyclic group (e.g., thiadiazolyl) optionally having one C₆₋₁₈ aryl; or
(e) a 8- to 12-membered fused non-aromatic heterocyclic group (e.g., dihydroindolyl), and
X¹ is a bond, or C₁₋₈ alkylene (e.g., —CH₂—, —CH(CH₃)—, —C(CH₃)₂—) optionally having 1 to 3 substituents selected from C₆₋₁₀ aryl and a halogen atom;
   (4) —X²—R⁵² wherein
R⁵² is a 8- to 12-membered fused non-aromatic heterocyclic group (e.g., dihydroindolyl), and
X² is C₁₋₆ alkylene (e.g., methylene); and
   (5) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and

X² is C₁₋₈ alkylene (e.g., methylene).

R⁵ is more preferably

(1) a 5- to 7-membered monocyclic aromatic heterocyclic group (e.g., thiazolyl, oxazolyl, pyrazolyl, oxadiazolyl) optionally having one substituent selected from
(a) C₆₋₁₀ aryl optionally having 1 to 3 halogen atoms,
(b) C₆₋₁₀ aryl-carbonyl (e.g., benzoyl, 1-naphthoyl) optionally having 1 to 3 substituents selected from
   (i) a halogen atom,
   (ii) C₁₋₈ alkyl optionally having 1 to 3 halogen atoms,
   (iii) C₁₋₆ alkoxy, and
   (iv) C₆₋₁₀ aryl,
(c) 5- to 7-membered monocyclic aromatic heterocyclyl-carbonyl (e.g., thienylcarbonyl, furylcarbonyl, pyrazolylcarbonyl, isooxazolylcarbonyl, pyrimidinylcarbonyl) optionally having 1 to 3 substituents selected from cyano and C₁₋₆ alkyl,
(d) 8- to 12-membered fused aromatic heterocyclyl-carbonyl (e.g., benzothienylcarbonyl),
(e) C₇₋₁₃ aralkyl optionally having 1 to 3 halogen atoms, and
(f) C₂₋₆ alkenyl-carbonyl (e.g., vinylcarbonyl, allylcarbonyl) optionally having one C₆₋₁₀ aryl;
   (2) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl, fluorenyl), which optionally has 1 to 3 substituents selected from
   (i) hydroxy,
   (ii) a halogen atom,
   (iii) C₁₋₆ alkyl-carbonyloxy (e.g., acetyloxy), and
   (iv) oxo;
(b) a monovalent group derived from a fused ring formed by condensation of tetrahydropyran or tetrahydrofuran and a benzene ring (e.g., 2,3-dihydrochromenyl, 2,3-dihydrobenzofuranyl), which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group (e.g., thiadiazolyl) optionally having one C₆₋₁₀ aryl; or
(e) dihydroindolyl, and
X¹ is
(a) a bond, or
(b) C₁₋₆ alkylene (e.g., —CH₂—, —CH(CH₃)—, —C(CH₃)₂—) optionally having 1 to 3 substituents selected from (i) C₆₋₁₀ aryl (e.g., phenyl) and (ii), a halogen atom (e.g., a fluorine atom);
   (3) —X²—R⁵² wherein
R⁵² is a 8- to 12-membered fused non-aromatic heterocyclic group (e.g., dihydroindolyl), and
X² is C₁₋₆ alkylene (e.g., methylene); or
   (4) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene).

As another embodiment, R⁵ is preferably

(1) a 5- to 7-membered monocyclic aromatic heterocyclic group (e.g., thiazolyl) optionally having one substituent selected from
(a) C₆₋₁₀ aryl-carbonyl (e.g., benzoyl, 1-naphthoyl) optionally having 1 to 3 halogen atoms, and
(b) 5- to 7-membered monocyclic aromatic heterocyclyl-carbonyl (e.g., thienylcarbonyl);
   (2) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl),
(b) a monovalent group derived from a fused ring formed by condensation of tetrahydropyran and a benzene ring (e.g., 2,3-dihydrochromenyl), which optionally has 1 to 3 halogen atoms, or
(c) C₆₋₁₀ aryl, and
X¹ is a bond, or C₁₋₆ alkylene (e.g., methylene);
   (3) —X²—R⁵² wherein
R⁵² is a 8- to 12-membered fused non-aromatic heterocyclic group (e.g., dihydroindolyl), and
X² is C₁₋₆ alkylene (e.g., methylene); or
   (4) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene).
R⁵ is still more preferably —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl, fluorenyl), which optionally has 1 to 3 substituents selected from hydroxy, a halogen atom, C₁₋₆ alkyl-carbonyloxy (e.g., acetyloxy) and oxo;
(b) a monovalent group derived from a fused ring formed by condensation of tetrahydropyran or tetrahydrofuran and a benzene ring (e.g., 2,3-dihydrochromenyl, 2,3-dihydrobenzofuranyl), which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group (e.g., thiadiazolyl) optionally having one C₆₋₁₀ aryl; or
(e) dihydroindolyl, and
X¹ is
(a) a bond, and
(b) C₁₋₆ alkylene (e.g., —CH₂—, —CH(CH₃)—, —C(CH₃)₂—) optionally having 1 to 3 substituents selected from C₆₋₁₀ aryl (e.g., phenyl) and a halogen atom (e.g., a fluorine atom).

R⁵ is particularly preferably —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl, fluorenyl), which optionally has 1 to 3 substituents selected from hydroxy, a halogen atom, C₁₋₈ alkyl-carbonyloxy (e.g., acetyloxy) and oxo; or
(b) a monovalent group derived from a fused ring formed by condensation of tetrahydropyran or tetrahydrofuran and a benzene ring (e.g., 2,3-dihydrochromenyl, 2,3-dihydrobenzofuranyl), which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group (e.g., thiadiazolyl) optionally having one C₆₋₁₀ aryl; or
(e) dihydroindolyl, and

X¹ is
(a) a bond, or
(b) C₁₋₈ alkylene (e.g., —CH₂—, —CH(CH₃)—, —C(CH₃)₂—) optionally having 1 to 3 substituents selected from C₆₋₁₀ aryl (e.g., phenyl) and a halogen atom (e.g., a fluorine atom).

A compound wherein R⁵ is —C(═O)NR⁵¹—X¹—R⁵² wherein
R⁵¹ is a hydrogen atom,
R⁵² is a monovalent group derived from a fused ring formed by condensation of tetrahydropyran and a benzene ring (e.g., 2,3-dihydrochromenyl), which optionally has 1 to 3 halogen atoms (e.g., a fluorine atom), and
X¹ is a bond,
   is preferable, since it is superior in metabolic stability.

A compound wherein the absolute configuration of the carbon atom on the pyrrolidine that R⁵ is bonded to is S-configuration is superior in the IAP antagonistic activity.

As another embodiment, the “cyclic group” of the “cyclic group optionally having substituent(s)” for R⁴ or the “chain aliphatic hydrocarbon group” of the “chain aliphatic hydrocarbon group optionally having substituent(s)” for R⁴ has, as a substituent, a group represented by the formula:

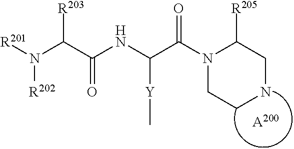

wherein Y is —Y²⁰¹—Y²⁰²—Y¹⁰³ mentioned below, and the other symbols are defined as below. Preferable examples of such embodiment include the below-mentioned compound (II).

Compound (II) is explained in the following.

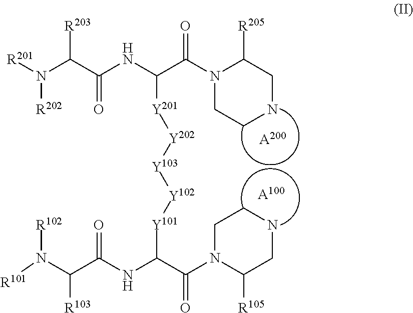

In the formula (II), ring A¹⁰⁰ is a nitrogen-containing heterocycle optionally having substituent(s).

Specific examples of ring A¹⁰⁰ include those exemplified as the above-mentioned ring A.

In the formula (II), ring A²⁰⁰ is a nitrogen-containing heterocycle optionally having substituent(s).

Specific examples of ring A²⁰⁰ include those exemplified as the above-mentioned ring A.

In the formula (II), R¹⁰¹ is a hydrogen atom, a chain aliphatic hydrocarbon group optionally having substituent(s), —OR¹¹¹, —C(═O)—R¹¹¹, —C(═O)—O—R¹¹¹, —N(R¹¹²)—R¹¹¹ or —C(═O)—N(R¹¹²)—R¹¹¹. As used herein, R¹¹¹ is a chain aliphatic hydrocarbon group optionally having substituent(s), and R¹¹² is a hydrogen atom or a chain aliphatic hydrocarbon group optionally having substituent(s).

Specific examples of R¹¹¹ include those exemplified as the above-mentioned R¹. As used herein, examples of R¹¹¹ include those exemplified as the above-mentioned R¹¹, and examples of R¹¹² include those exemplified as the above-mentioned R¹².

In the formula (II), R²⁰¹ is a hydrogen atom, a chain aliphatic hydrocarbon group optionally having substituent(s), —C(═O)—R²¹¹, —C(═O)—N(R²¹²)—R²¹¹ or —C(═O)—N(R²¹²)—R²¹¹. As used herein, R²¹¹ is a chain aliphatic hydrocarbon group optionally having substituent(s), and R²¹² is a hydrogen atom or a chain aliphatic hydrocarbon group optionally having substituent(s).

Specific examples of R²⁰¹ include those exemplified as the above-mentioned R¹. As used herein, examples of R²¹¹ include those exemplified as the above-mentioned R¹¹, and examples of R²¹² include those exemplified as the above-mentioned R¹².

In the formula (II), R¹⁰² is a hydrogen atom, a chain aliphatic hydrocarbon group optionally having substituent(s), —C(═O)—R¹²¹, —C(═O)—O—R¹²¹ or —C(═O)—N(R¹²²)—R¹²¹. As used herein, R¹²¹ is a chain aliphatic hydrocarbon group optionally having substituent(s), and R¹²² is a hydrogen atom or a chain aliphatic hydrocarbon group optionally having substituent(s).

Specific examples of R¹⁰² include those exemplified as the above-mentioned R². As used herein, examples of R¹²¹ include those exemplified as the above-mentioned R²¹, and examples of R¹²² include those exemplified as the above-mentioned R²².

In the formula (II), R²⁰² is a hydrogen atom, a chain aliphatic hydrocarbon group optionally having substituent(s), —C(═O)—R²²¹, —C(═O)—O—R²²¹ or —C(═O)—N(R²²²)—R²²¹. As used herein, R²²¹ is a chain aliphatic hydrocarbon group optionally having substituent(s), and R²²² is a hydrogen atom or a chain aliphatic hydrocarbon group optionally having substituent(s).

Specific examples of R²⁰² include those exemplified as the above-mentioned R². As used herein, examples of R²²¹ include those exemplified as the above-mentioned R²¹, and examples of R²²² include those exemplified as the above-mentioned R²².

In the formula (II), R¹⁰³ is a hydrogen atom or a chain aliphatic hydrocarbon group optionally having substituent(s).

Specific examples of R¹⁰³ include those exemplified as the above-mentioned R³.

As another embodiment, R¹⁰¹ and R¹⁰² in combination form, together with the adjacent nitrogen atom, a 4- to 7-membered nitrogen-containing non-aromatic heterocycle optionally having substituent(s).

As another embodiment, R¹⁰¹ and R¹⁰³ in combination form, together with the adjacent nitrogen atom, a 4- to 7-membered nitrogen-containing non-aromatic heterocycle optionally having substituent(s).

In the formula (II), R²⁰³ is a hydrogen atom or a chain aliphatic hydrocarbon group optionally having substituent(s).

Specific examples of R²⁰³ include those exemplified as the above-mentioned R³.

As another embodiment, R²⁰¹ and R²⁰² in combination form, together with the adjacent nitrogen atom, a 4- to 7-membered nitrogen-containing non-aromatic heterocycle optionally having substituent(s).

As another embodiment, R²⁰¹ and R²⁰³ in combination form, together with the adjacent nitrogen atom, a 4- to 7-membered nitrogen-containing non-aromatic heterocycle optionally having substituent(s).

In the formula (II), R¹⁰⁵ is a cyclic group optionally having substituent(s), —C(═O)—NR¹⁵¹—X¹⁰¹—R¹⁵², —C(═S)—NR¹⁵¹—X¹⁰¹—R¹⁵², —X¹⁰²—R¹⁵², —X¹⁰²—NR¹⁵¹R¹⁵³, —X¹⁰²—NR¹⁵¹—C(═O)—R¹⁵³ or —C(═NR¹⁵⁴)—NR¹⁵¹—X¹⁰¹—R¹⁵². As used herein, X¹⁰¹ is a bond or C₁₋₆ alkylene optionally having substituent(s), X¹⁰² is C₁₋₆ alkylene optionally having substituent(s), R¹⁵¹ is a hydrogen atom, a chain aliphatic hydrocarbon group optionally having substituent(s) or a cyclic group optionally having substituent(s), R¹⁵² is a cyclic group optionally having substituent(s), R¹⁵³ is a chain aliphatic hydrocarbon group optionally having substituent(s) or a cyclic group optionally having substituent(s), and R¹⁵⁴ is a hydrogen atom or a chain aliphatic hydrocarbon group optionally having substituent(s).

Specific examples of R¹⁰⁵ include those exemplified as the above-mentioned R⁵. As used herein, examples of X¹⁰¹ include those exemplified as the above-mentioned X¹, examples of X¹⁰² include those exemplified as the above-mentioned X², examples of R¹⁵¹ include those exemplified as the above-mentioned R⁵¹, examples of R¹⁵² include those exemplified as the above-mentioned R⁵², examples of R¹⁵³ include those exemplified as the above-mentioned R⁵³, and examples of R¹⁵⁴ include those exemplified as the above-mentioned R⁵⁴.

In the formula (II), R²⁰⁵ is a cyclic group optionally having substituent(s), —C(═O)—NR²⁵¹—X²⁰¹—R²⁵², —C(═S)—NR²⁵¹—X²⁰¹—R²⁵², —X²⁰²—R²⁵², —X²⁰²—NR²⁵¹R²⁵³, —X²⁰²—NR²⁵¹—C(═O)—R²⁵³ or —C(═NR²⁵⁴)—NR²⁵¹—X²⁰¹—R²⁵². As used herein, X²⁰¹ is a bond or C₁₋₆ alkylene optionally having substituent(s), X²⁰² is C₁₋₆ alkylene optionally having substituent(s), R²⁵¹ is a hydrogen atom, a chain aliphatic hydrocarbon group optionally having substituent(s) or a cyclic group optionally having substituent(s), R²⁵² is a cyclic group optionally having substituent(s), R²⁵³ is a chain aliphatic hydrocarbon group optionally having substituent(s) or a cyclic group optionally having substituent(s), and R²⁵⁴ is a hydrogen atom or a chain aliphatic hydrocarbon group optionally having substituent(s).

Specific examples of R²⁰⁵ include those exemplified as the above-mentioned R⁵. As used herein, examples of X²⁰¹ include those exemplified as the above-mentioned X¹, examples of X²⁰² include those exemplified as the above-mentioned X², examples of R²⁵¹ include those exemplified as the above-mentioned R⁵¹, examples of R²⁵² include those exemplified as the above-mentioned R⁵², examples of R²⁵³ include those exemplified as the above-mentioned R⁵³, and examples of R²⁵⁴ include those exemplified as the above-mentioned R⁵⁴.

In the formula (II), —Y¹⁰¹— is a divalent chain aliphatic hydrocarbon group optionally having substituent(s) or a divalent cyclic group optionally having substituent(s).

Examples of the “divalent chain aliphatic hydrocarbon group optionally having substituent(s)” for —Y¹⁰¹— include C₁₋₆ alkylene optionally having substituent(s), C₂₋₆ alkenylene optionally having substituent(s), C₂₋₆ alkynylene optionally having substituent(s) and the like.

Examples of the “divalent cyclic group optionally having substituent(s)” for —Y¹⁰¹— include C₃₋₈ cycloalkylene optionally having substituent(s), a divalent group derived from a fused ring formed by condensation of C₃₋₆ cycloalkane and a benzene ring, which optionally has substituent(s), C₆₋₁₀ arylene optionally having substituent(s), a divalent aromatic heterocyclic group optionally having substituent(s), a divalent non-aromatic heterocyclic group optionally having substituent(s) and the like.

In the formula (II), —Y²⁰¹— is a divalent chain aliphatic hydrocarbon group optionally having substituent(s) or a divalent cyclic group optionally having substituent(s).

Examples of the “divalent chain aliphatic hydrocarbon group optionally having substituent(s)” for include C₁₋₆ alkylene optionally having substituent(s), C₂₋₆ alkenylene optionally having substituent(s), C₂₋₆ alkynylene optionally having substituent(s) and the like.

Examples of the “divalent cyclic group optionally having substituent(s)” for —Y²⁰¹— include C₃₋₈ cycloalkylene optionally having substituent(s), a divalent group derived from a fused ring formed by condensation of C₃₋₆ cycloalkane and a benzene ring, which optionally has substituent(s), C₆₋₁₀ arylene optionally having substituent(s), a divalent aromatic heterocyclic group optionally having substituent(s), a divalent non-aromatic heterocyclic group optionally having substituent(s) and the like.

In the formula (II), —Y¹⁰²— is a bond, —C(═O)—, —C(═O)—O—, —C(═O)—NR¹⁰⁶—, —O—, —O—C(═O)—, —O—C(═O)—NR¹⁰⁶—, —NR¹⁰⁶—, —NR¹⁰⁶—C(═O)—NR¹⁰⁶—C(═O)—O—, —NR¹⁰⁶—S(═O)₂—, —S—, —S(═O)—, —S(═O)₂— or —S(═O)₂—NR¹⁰⁶—. As used herein, R¹⁰⁶ is a hydrogen atom or a chain aliphatic hydrocarbon group optionally having substituent(s).

In one embodiment, when —Y¹⁰¹— is a divalent nitrogen-containing heterocyclic group optionally having substituent(s), and is bonded to —Y¹⁰²— at the nitrogen atom constituting the ring, then —Y¹⁰²— is a bond, —C(═O)—, —C(═O)—O—, —C(═O)—NR¹⁰⁶—, —S(═O)₂— or —S(═O)₂—NR¹⁰⁶—.

Examples of the “divalent nitrogen-containing heterocyclic group” of the above-mentioned “divalent nitrogen-containing heterocyclic group optionally having substituent(s)” include a group wherein at least one nitrogen atom(s) constituting the ring has a bond, from among divalent groups derived from the above-mentioned nitrogen-containing heterocycle. Specific examples thereof include the following:

(1) 5- to 7-membered (preferably 5- or 6-membered) divalent monocyclic nitrogen-containing aromatic heterocyclic groups (at least one nitrogen atom(s) constituting the ring has a bond), for example, pyrrole-1,2-diyl, pyrrole-1,3-diyl, imidazole-1,2-diyl, imidazole-1,4-diyl, imidazole-1,5-diyl, pyrazole-1,3-diyl, pyrazole-1,4-diyl, pyrazole-1,5-diyl, 1,2,3-triazole-1,4-diyl, 1,2,3-triazole-1,5-diyl, 1,2,4-triazole-1,3-diyl, tetrazole-1,5-diyl);
(2) 8- to 12-membered divalent fused nitrogen-containing aromatic heterocyclic groups (at least one nitrogen atom(s) constituting the ring has a bond), for example, 1H-benzimidazole-1,2-diyl, 1H-benzimidazole-1,4-diyl, 1H-benzimidazole-1,5-diyl, 1H-benzimidazole-1,6-diyl, 1H-benzimidazole-1,7-diyl, 1H-1,2,3-benzotriazole-1,4-diyl, 1H-1,2,3-benzotriazole-1,5-diyl, 1H-1,2,3-benzotriazole-1,6-diyl, 1H-1,2,3-benzotriazole-1,7-diyl, indole-1,2-diyl, indole-1,3-diyl, indole-1,4-diyl, indole-1,5-diyl, indole-1,6-diyl, indole-1,7-diyl, 1H-imidazo[4,5-b]pyridine-1,2-diyl, 1H-imidazo[4,5-b]pyridine-1,5-diyl, 1H-imidazo[4,5-b]pyridine-1,6-diyl, 1H-imidazo[4,5-b]pyridine-1,7-diyl, 1H-imidazo[4,5-c]pyridine-1,2-diyl, 1H-imidazo[4,5-c]pyridine-1,4-diyl, 1H-imidazo[4,5-c]pyridine-1,6-diyl, 1H-imidazo[4,5-c]pyridine-1,7-diyl;
(3) 4- to 7-membered (preferably 5- or 6-membered) divalent monocyclic nitrogen-containing non-aromatic heterocyclic groups (at least one nitrogen atom(s) constituting the ring has a bond), for example, azetidine-1,2-diyl, azetidine-1,3-diyl, pyrrolidine-1,2-diyl, pyrrolidine-1,3-diyl, imidazolidine-1,2-diyl, imidazolidine-1,3-diyl, imidazolidine-1,4-diyl, pyrazolidine-1,2-diyl, pyrazolidine-1,3-diyl, pyrazolidine-1,4-diyl, piperidine-1,2-diyl, piperidine-1,3-diyl, piperidine-1,4-diyl, piperazine-1,2-diyl, piperazine-1,3-diyl, piperazine-1,4-diyl, morpholine-2,4-diyl, morpholine-3,4-diyl; and
(4) 8- to 12-membered fused nitrogen-containing non-aromatic heterocyclic groups (at least one nitrogen atom(s) constituting the ring has a bond), for example, indoline-1,2-diyl, indoline-1,3-diyl, indoline-1,4-diyl, indoline-1,5-diyl, indoline-1,6-diyl, indoline-1,7-diyl, isoindoline-1,2-diyl, isoindoline-2,3-diyl, isoindoline-2,4-diyl, isoindoline-2,5-diyl, isoindoline-2,6-diyl, isoindoline-2,7-diyl, 1,2-dihydroquinoline-1,2-diyl, 1,2-dihydroquinoline-1,3-diyl, 1,2-dihydroquinoline-1,4-diyl, 1,2-dihydroquinoline-1,5-diyl, 1,2-dihydroquinoline-1,6-diyl, 1,2-dihydroquinoline-1,7-diyl, dihydroquinoline-1,8-diyl, 1,2,3,4-tetrahydroquinoline-1,2-diyl, 1,2,3,4-tetrahydroquinoline-1,3-diyl, 1,2,3,4-tetrahydroquinoline-1,4-diyl, 1,2,3,4-tetrahydroquinoline-1,5-diyl, 1,2,3,4-tetrahydroquinoline-1,6-diyl, 1,2,3,4-tetrahydroquinoline-1,7-diyl, 1,2,3,4-tetrahydroquinoline-1,8-diyl, 1,2-dihydroisoquinoline-1,2-diyl, 1,2-dihydroisoquinoline-2,3-diyl, 1,2-dihydroisoquinoline-2,4-diyl, 1,2-dihydroisoquinoline-2,5-diyl, 1,2-dihydroisoquinoline-2,6-diyl, 1,2-dihydroisoquinoline-2,7-diyl, 1,2-dihydroisoquinoline-2,8-diyl, 1,2,3,4-tetrahydroisoquinoline-1,2-diyl, 1,2,3,4-tetrahydroisoquinoline-2,3-diyl, 1,2,3,4-tetrahydroisoquinoline-2,4-diyl, 1,2,3,4-tetrahydroisoquinoline-2,5-diyl, 1,2,3,4-tetrahydroisoquinoline-2,6-diyl, 1,2,3,4-tetrahydroisoquinoline-2,7-diyl, 1,2,3,4-tetrahydroisoquinoline-2,8-diyl.

When the “divalent nitrogen-containing heterocyclic group” of the above-mentioned “divalent nitrogen-containing heterocyclic group optionally having substituent(s)” is a divalent aromatic heterocyclic group (e.g., a 5- to 7-membered divalent monocyclic nitrogen-containing aromatic heterocyclic group, a 8- to 12-membered divalent fused nitrogen-containing aromatic heterocyclic group), examples of the substituent that the divalent aromatic heterocyclic group optionally has include substituents selected from the above-mentioned Substituent Group B. The number of the substituents is not particularly limited as long as it is a substitutable number, which is preferably 1 to 5, more preferably 1 to 3. When plural substituents are present, they may be the same or different.

When the “divalent nitrogen-containing heterocyclic group” of the above-mentioned “divalent nitrogen-containing heterocyclic group optionally having substituent(s)” is a divalent non-aromatic heterocyclic group (e.g., a 4- to 7-membered divalent monocyclic nitrogen-containing non-aromatic heterocyclic group, a 8- to 12-membered divalent fused nitrogen-containing non-aromatic heterocyclic group), examples of the substituent that the divalent non-aromatic heterocyclic group optionally has include substituents selected from the above-mentioned Substituent Group C. The number of the substituents is not particularly limited as long as it is a substitutable number, which is preferably 1 to 5, more preferably 1 to 3. When plural substituents are present, they may be the same or different.

In the formula (II), —Y²⁰²— is a bond, —C(═O)—, —C(═O)—O—, —C(═O)—NR²⁰⁶—, —O—, —O—C(═O)—, —O—C(═O)—NR²⁰⁶—, —NR²⁰⁶—, —NR²⁰⁶—C(═O)—, —NR²⁰⁶—C(═O)—O—, —NR²⁰⁶—S(═O)₂—, —S—, —S(═O)—, —S(═O)₂— or —S(═O)₂—NR²⁰⁶—. As used herein, R²⁰⁶ is a hydrogen atom or a chain aliphatic hydrocarbon group optionally having substituent(s).

In one embodiment, when —Y²⁰¹— is a divalent nitrogen-containing heterocyclic group optionally having substituent(s), and is bonded to —Y²⁰²— at the nitrogen atom constituting the ring, then —Y²⁰²— is a bond, —C(═O)—, —C(═O)—O—, —C(═O)—NR²⁰⁶—, —S(═O)₂— or —S(═O)₂—NR²⁰⁶—.

Examples of the above-mentioned “divalent nitrogen-containing heterocyclic group optionally having substituent(s)” include those exemplified for the above-mentioned —Y¹⁰¹—.

—Y¹⁰³— is a bond or a spacer having 1 to 20 atoms in the main chain.

The “main chain” of the “spacer having 1 to 20 atoms in the main chain” means is a divalent straight chain connecting —Y¹⁰²— and —Y²⁰²—, and the atom number of the main chain is counted such that the number of atoms in the main chain will be minimum.

Specific examples of the “spacer having 1 to 20 atoms in the main chain” include

(1) C₁₋₆ alkylene optionally having substituent(s);
(2) C₂₋₆ alkenylene optionally having substituent(s);
(3) C₂₋₆ alkynylene optionally having substituent(s);
(4) C₃₋₆ cycloalkylene optionally having substituent(s);
(5) C₆₋₁₀ arylene optionally having substituent(s);
(6) a divalent group derived from a fused ring formed by condensation of C₃₋₆ cycloalkane and a benzene ring, which optionally has substituent(s);
(7) a divalent aromatic heterocyclic group optionally having substituent(s);
(8) a divalent non-aromatic heterocyclic group optionally having substituent(s);
(9) a divalent group having 1 to 20 atoms in the main chain formed by combination of 2 to 4 kinds of the above-mentioned (1) to (8).

Preferable specific examples of compound (I) include the following.

### Compound (A1a)

A compound of the formula (I) wherein

ring A is
(1) a 5- to 7-membered (preferably 5- or 6-membered) monocyclic nitrogen-containing aromatic heterocycle having “—CH₂—NH—” or “═CH—NH—” as a ring-constituting member (e.g., pyrrole, imidazole, pyrazole, triazole (e.g., 1,2,3-triazole, 1,2,4-triazole), tetrazole) and optionally having 1 to 5 (preferably 1 to 3) substituents selected from
(a) a halogen atom,
(b) hydroxy,
(c) C₁₋₆ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy), and
(f) C₇₋₁₃ aralkyloxy (e.g., benzyloxy),
   (2) a 8- to 12-membered fused nitrogen-containing aromatic heterocycle having “—CH₂—NH—” or “═CH—NH—” as a ring-constituting member (e.g., benzimidazole (e.g., 1H-benzimidazole), benzotriazole (e.g., 1H-1,2,3-benzotriazole), indole, imidazopyridine (e.g., 1H-imidazo[4,5-b]pyridine, 1H-imidazo[4,5-c]pyridine)) and optionally having 1 to 5 (preferably 1 to 3) substituents selected from
(a) a halogen atom,
(b) hydroxy,
(c) C₁₋₈ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy), and
(f) C₇₋₁₃ aralkyloxy (e.g., benzyloxy),
   (3) a 4- to 7-membered (preferably 5- or 6-membered) monocyclic nitrogen-containing non-aromatic heterocycle having “—CH₂—NH—” or “═CH—NH—” as a ring-constituting member (e.g., azetidine, pyrrolidine, imidazolidine, pyrazolidine, piperidine, piperazine, morpholine) and optionally having 1 to 5 (preferably 1 to 3) substituents selected from
(a) a halogen atom,
(b) hydroxy,
(c) C₁₋₈ alkoxy optionally having halogen atom(s),
(d) C₃₋₈ cycloalkyloxy (e.g., cyclopropoxy, cyclopentyloxy),
(e) C₆₋₁₀ aryloxy (e.g., phenyloxy, naphthyloxy),
(f) C₇₋₁₃ aralkyloxy (e.g., benzyloxy), and
(g) oxo, or
   (4) a 6- to 12-membered fused nitrogen-containing non-aromatic heterocycle having “—CH₂—NH—” or “═CH—NH—” as a ring-constituting member (e.g., dihydroindole (e.g., indoline), dihydroisoindole (e.g., isoindoline), dihydroquinoline (e.g., 1,2-dihydroquinoline), tetrahydroquinoline (e.g., 1,2,3,4-tetrahydroquinoline), dihydroisoquinoline (e.g., 1,2-dihydroisoquinoline), tetrahydroisoquinoline (e.g., 1,2,3,4-tetrahydroisoquinoline), azabicyclohexane (e.g., 2-azabicyclo[3.1.0]hexane)) and optionally having 1 to 5 (preferably 1 to 3) substituents selected from
(a) a halogen atom,
(b) hydroxy,
(c) C₁₋₆ alkoxy optionally having halogen atom(s),
(d) C₃₋₈ cycloalkyloxy (e.g., cyclopropoxy, cyclopentyloxy),
(e) C₆₋₁₀ aryloxy (e.g., phenyloxy, naphthyloxy),
(f) C₇₋₁₃ aralkyloxy (e.g. benzyloxy), and
(g) oxo;
   R¹ is C₁₋₆ alkyl (e.g., methyl) optionally having substituent(s);
   R² is a hydrogen atom;
   R³ is C₁₋₆ alkyl (e.g., methyl) optionally having substituent(s);

### R⁴ is

(1) C₃₋₉ cycloalkyl (e.g., cyclohexyl) optionally having substituent(s);
(2) a 4- to 7-membered monocyclic non-aromatic heterocyclic group (e.g., tetrahydropyranyl, piperidyl, 1,1-dioxidotetrahydrothiopyranyl) optionally having substituent(s);
(3) C₁₋₆ alkyl (e.g., propyl, butyl) optionally having substituent(s); or
(4) C₆₋₁₀ aryl (e.g., phenyl) optionally having substituent(s); and

### R⁵ is

(1) a 5- to 7-membered monocyclic aromatic heterocyclic group (e.g., thiazolyl) optionally having substituent(s);
(2) a 8- to 12-membered fused aromatic heterocyclic group (e.g., benzothiazolyl) optionally having substituent(s);
(3) —C(═O)NR⁵¹—X¹—R⁵² wherein R⁵¹, X¹ and R⁵² are defined above;
(4) —X²—R⁵² wherein X² and R⁵² are defined above; or
(5) —X²—NR⁵¹R⁵³ wherein R⁵¹, X² and R⁵³ are defined above, or a salt thereof.

### Compound (A1)

A compound of the formula (I) wherein

ring A is
(1) a 5- to 7-membered (preferably 5- or 6-membered) monocyclic nitrogen-containing aromatic heterocycle having “—CH₂—NH—” or “═CH—NH—” as a ring-constituting member (e.g., pyrrole, imidazole, pyrazole, triazole (e.g., 1,2,3-triazole, 1,2,4-triazole), tetrazole) and optionally having 1 to 5 (preferably 1 to 3) substituents selected from
(a) a halogen atom,
(b) hydroxy,
(c) C₁₋₆ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy), and
(f) C₇₋₁₃ aralkyloxy (e.g., benzyloxy),
   (2) a 8- to 12-membered fused nitrogen-containing aromatic heterocycle having “—CH₂—NH—” or “—CH—NH—” as a ring-constituting member (e.g., benzimidazole (e.g., 1H-benzimidazole), benzotriazole (e.g., 1H-1,2,3-benzotriazole), indole, imidazopyridine (e.g., 1H-imidazo[4,5-b]pyridine, 1H-imidazo[4,5-c]pyridine)) and optionally having 1 to 5 (preferably 1 to 3) substituents selected from
(a) a halogen atom,
(b) hydroxy,
(c) C₁₋₆ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy), and
(f) C₇₋₁₃ aralkyloxy (e.g., benzyloxy),
   (3) a 4- to 7-membered (preferably 5- or 6-membered) monocyclic nitrogen-containing non-aromatic heterocycle having “—CH₂—NH—” or “═CH—NH—” as a ring-constituting member (e.g., azetidine, pyrrolidine, imidazolidine, pyrazolidine, piperidine, piperazine, morpholine) and optionally having 1 to 5 (preferably 1 to 3) substituents selected from
(a) a halogen atom,
(b) hydroxy,
(c) C₁₋₆ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy),
(f) C₇₋₁₃ aralkyloxy (e.g., benzyloxy), and
(g) oxo, or
   (4) a 8- to 12-membered fused nitrogen-containing non-aromatic heterocycle having “—CH₂—NH—” or “═CH—NH—” as a ring-constituting member (e.g., dihydroindole (e.g., indoline), dihydroisoindole (e.g., isoindoline), dihydroquinoline (e.g., 1,2-dihydroquinoline), tetrahydroquinoline (e.g., 1,2,3,4-tetrahydroquinoline), dihydroisoquinoline (e.g., 1,2-dihydroisoquinoline), tetrahydroisoquinoline (e.g., 1,2,3,4-tetrahydroisoquinoline), azabicyclohexane (e.g., 2-azabicyclo[3.1.0]hexane)) and optionally having 1 to 5 (preferably 1 to 3) substituents selected from
(a) a halogen atom,
(b) hydroxy,
(c) C₁₋₆ alkoxy,
(d) C₃₋₆ cycloalkyloxy (e.g., cyclopropoxy, cyclopentyloxy),
(e) C₆₋₁₀ aryloxy (e.g., phenyloxy, naphthyloxy),
(f) aralkyloxy (e.g., benzyloxy), and
(g) oxo;
   R¹ is C₁₋₆ alkyl (e.g., methyl) optionally having substituent(s);
   R² is a hydrogen atom;
   R³ is C₁₋₆ alkyl (e.g., methyl) optionally having substituent(s);
   R⁴ is (1) C₃₋₈ cycloalkyl (e.g., cyclohexyl) optionally having substituent(s);
   (2) a 4- to 7-membered monocyclic non-aromatic heterocyclic group (e.g., tetrahydropyranyl, piperidyl, 1,1-dioxidotetrahydrothiopyranyl) optionally having substituent(s); or
   (3) C₁₋₈ alkyl (e.g., propyl, butyl) optionally having substituent(s); and

### R⁵ is

(1) a 5- to 7-membered monocyclic aromatic heterocyclic group (e.g., thiazolyl) optionally having substituent(s);
(2) a 8- to 12-membered fused aromatic heterocyclic group (e.g., benzothiazolyl) optionally having substituent(s);
(3) —C(═O)NR⁵¹—X¹—R⁵² wherein R⁵¹, X¹ and R⁵² are defined above;
(4) —X²—R⁵² wherein X² and R⁵² are defined above; or
(5) —X²—NR⁵¹R⁵³ wherein R⁵¹, X² and R⁵³ are defined above, or a salt thereof.

### Compound (A2a)

A compound of the formula (I) wherein

ring A is pyrrolidine, dihydroindole (e.g., indoline) or azabicyclohexane (e.g., 2-azabicyclo[3.1.0]hexane) optionally having 1 to 5 (preferably 1 to 3) substituents selected from
(a) a halogen atom,
(b) hydroxy,
(c) C₁₋₈ alkoxy optionally having halogen atom(s),
(d) C₃₋₈ cycloalkyloxy (e.g., cyclopropoxy, cyclopentyloxy),
(e) C₆₋₁₀ aryloxy (e.g., phenyloxy, naphthyloxy),
(f) C₇₋₁₃ aralkyloxy (e.g., benzyloxy), and
(g) oxo;
   R¹ is C₁₋₆ alkyl (e.g., methyl) optionally having 1 to 3 substituents selected from
(1) amino, and
(2) imino;
   R² is a hydrogen atom;
   R³ is C₁₋₈ alkyl (e.g., methyl);

### R⁴ is

(1) C₃₋₈ cycloalkyl (e.g., cyclohexyl) optionally having 1 to 3 substituents selected from
(a) a halogen atom,
(b) hydroxy,
(c) C₁₋₆ alkoxy-carbonyl (e.g., methoxycarbonyl) optionally having one C₆₋₁₀ aryl (e.g., phenyl), and
(d) oxo;
   (2) a 4- to 7-membered monocyclic non-aromatic heterocyclic group (e.g., tetrahydropyranyl, piperidyl, 1,1-dioxidotetrahydrothiopyranyl);
   (3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxycarbonyl (e.g., methoxycarbonyl) optionally having one C₆₋₁₀ aryl (e.g., phenyl), and
(c) C₆₋₁₀ arylsulfonyl (e.g., phenylsulfonyl); or
   (4) C₆₋₁₀ aryl (e.g., phenyl); and R⁵ is (1) a 5- to 7-membered monocyclic aromatic heterocyclic group (e.g., thiazolyl) optionally having one substituent selected from
(a) C₆₋₁₀ aryl,
(b) C₆₋₁₀ aryl-carbonyl (e.g., benzoyl, 1-naphthoyl) optionally having 1 to 3 halogen atoms, and
(c) 5- to 7-membered monocyclic aromatic heterocyclyl-carbonyl (e.g., thienylcarbonyl);
   (2) a 8- to 12-membered fused aromatic heterocyclic group (e.g., benzothiazolyl) optionally having one substituent selected from
(a) C₆₋₁₀ aryl,
(b) C₆₋₁₀ aryl-carbonyl (e.g., benzoyl, 1-naphthoyl) optionally having 1 to 3 halogen atoms, and
(c) 5- to 7-membered monocyclic aromatic heterocyclyl-carbonyl (e.g., thienylcarbonyl);
   (3) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl), which optionally has 1 to 3 hydroxy;
(b) a monovalent group derived from a fused ring formed by condensation of tetrahydropyran and a benzene ring (e.g., 2,3-dihydrochromenyl), which optionally has 1 to 3 halogen atoms; or
(c) C₆₋₁₀ aryl, and
X¹ is a bond or C₁₋₆ alkylene (e.g., methylene);
   (4) —X²—R⁵² wherein
R⁵² a 8- to 12-membered fused non-aromatic heterocyclic group (e.g., dihydroindolyl), and
X² is C₁₋₆ alkylene (e.g., methylene); or
   (5) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene), or a salt thereof.

### Compound (A2)

A compound of the formula (I) wherein

ring A is pyrrolidine, dihydroindole (e.g., indoline) or azabicyclohexane (e.g., 2-azabicyclo[3.1.0]hexane), each optionally having 1 to 5 (preferably 1 to 3) substituents selected from
(a) a halogen atom,
(b) hydroxy,
(c) C₁₋₆ alkoxy,
(d) C₃₋₈ cycloalkyloxy (e.g., cyclopropoxy, cyclopentyloxy),
(e) C₆₋₁₀ aryloxy (e.g., phenyloxy, naphthyloxy),
(f) C₇₋₁₃ aralkyloxy (e.g., benzyloxy), and
(g) oxo;
   R¹ is C₁₋₆ alkyl (e.g., methyl) optionally having 1 to 3 substituents selected from
(1) amino, and
(2) imino;
   R² is a hydrogen atom;
   R³ is C₁₋₆ alkyl (e.g., methyl);

### R⁴ is

(1) C₃₋₈ cycloalkyl (e.g., cyclohexyl) optionally having 1 to 3 substituents selected from
(a) hydroxy,
(b) C₁₋₆ alkoxy-carbonyl (e.g., methoxycarbonyl) optionally having one C₆₋₁₀ aryl (e.g., phenyl), and
(c) oxo;
   (2) a 4- to 7-membered monocyclic non-aromatic heterocyclic group (e.g., tetrahydropyranyl, piperidyl, 1,1-dioxidotetrahydrothiopyranyl); or
   (3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl); and

### R⁵ is

(1) a 5- to 7-membered monocyclic aromatic heterocyclic group (e.g., thiazolyl) optionally having one substituent selected from
(a) C₆₋₁₀ aryl,
(b) C₆₋₁₀ aryl-carbonyl (e.g., benzoyl, 1-naphthoyl) optionally having 1 to 3 halogen atoms, and
(c) 5- to 7-membered monocyclic aromatic heterocyclyl-carbonyl (e.g., thienylcarbonyl);
   (2) a 8- to 12-membered fused aromatic heterocyclic group (e.g., benzothiazolyl) optionally having one substituent selected from
(a) C₆₋₁₀ aryl,
(b) C₆₋₁₀ aryl-carbonyl (e.g., benzoyl, 1-naphthoyl) optionally having 1 to 3 halogen atoms, and
(c) 5- to 7-membered monocyclic aromatic heterocyclyl-carbonyl (e.g., thienylcarbonyl);
   (3) —C(═O)NR⁵¹—X¹—R⁵² wherein
R⁵¹ is a hydrogen atom,
R⁵² is a monovalent group derived from a fused ring formed by condensation of C₃₋₈ cycloalkane and a benzene ring (e.g., 1,2,3,4-tetrahydronaphthyl), or C₆₋₁₀ aryl, and
X¹ is a bond, or C₁₋₈ alkylene (e.g., methylene);
   (4) —X²—R⁵² wherein
R⁵² is a 8- to 12-membered fused non-aromatic heterocyclic group (e.g., dihydroindolyl), and
X² is C₁₋₈ alkylene (e.g., methylene); or
   (5) —X²—NR⁵¹R⁵³ wherein
R⁵¹ is a hydrogen atom.
R⁵³ is a monovalent group derived from a fused ring formed by condensation of C₃₋₈ cycloalkane and a benzene ring (e.g., 1,2,3,4-tetrahydronaphthyl), and
X² is C₁₋₈ alkylene (e.g., methylene),
   or a salt thereof.

### Compound (A3a)

A compound of the formula (I) wherein

ring A is pyrrolidine optionally having 1 to 3 substituents selected from C₁₋₈ alkoxy (e.g., ethoxy) optionally having halogen atom(s), and a halogen atom;
R¹ is C₁₋₈ alkyl (e.g., methyl);
R² is a hydrogen atom;
R³ is C₁₋₈ alkyl (e.g., methyl);

### R⁴ is

(1) C₃₋₈ cycloalkyl (e.g., cyclohexyl) optionally having halogen atom(s);
(2) a 4- to 7-membered monocyclic non-aromatic heterocyclic as group (e.g., tetrahydropyranyl); or
(3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl); and

### R⁵ is

(1) a 5- to 7-membered monocyclic aromatic heterocyclic group (e.g., thiazolyl) optionally having one substituent selected from
(a) C₆₋₁₀ aryl-carbonyl (e.g., benzoyl, 1-naphthoyl) optionally having 1 to 3 halogen atoms, and
(b) 5- to 7-membered monocyclic aromatic heterocyclyl-carbonyl (e.g., thienylcarbonyl);
   (2) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl);
(b) a monovalent group derived from a fused ring formed by condensation of tetrahydropyran and a benzene ring (e.g., 2,3-dihydrochromenyl) which optionally has 1 to 3 halogen atoms; or
(c) C₆₋₁₀ aryl, and
X¹ is a bond, or C₁₋₆ alkylene (e.g., methylene);
   (3) —X²—R⁵² wherein
R⁵² is a 8- to 12-membered fused non-aromatic heterocyclic group (e.g., dihydroindolyl), and
X² is C₁₋₆ alkylene (e.g., methylene); or
   (4) —X²—NR⁵¹R⁵³ wherein
R³¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene), or a salt thereof.

### Compound (A3)

A compound of the formula (I) wherein

ring A is pyrrolidine;
R¹ is C₁₋₆ alkyl (e.g., methyl);
R² is a hydrogen atom;
R³ is C₁₋₆ alkyl (e.g., methyl);

### R⁴ is

(1) C₃₋₉ cycloalkyl (e.g., cyclohexyl);
(2) a 4- to 7-membered monocyclic non-aromatic heterocyclic group (e.g., tetrahydropyranyl); or
(3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) C₆₋₁₀ arylsulfonyl (e.g., phenylsulfonyl); and

### R⁵ is

(1) a 5- to 7-membered monocyclic aromatic heterocyclic group (e.g., thiazolyl) optionally having one substituent selected from
(a) C₆₋₁₀ aryl-carbonyl (e.g., benzoyl, 1-naphthoyl) optionally having 1 to 3 halogen atoms, and
(b) 5- to 7-membered monocyclic aromatic heterocyclyl-carbonyl (e.g., thienylcarbonyl);
   (2) —C(═O)NR⁵¹—X¹—R⁵² wherein
R⁵¹ is a hydrogen atom,
R⁵² is a monovalent group derived from a fused ring formed by condensation of C₃₋₈ cycloalkane and a benzene ring (e.g., 1,2,3,4-tetrahydronaphthyl), or C₆₋₁₀ aryl, and
X¹ is a bond or C₁₋₆ alkylene (e.g., methylene);
   (3) —X²—R⁵² wherein
R⁵² is a 8- to 12-membered fused non-aromatic heterocyclic group (e.g., dihydroindolyl), and
X² is C₁₋₆ alkylene (e.g., methylene); or
   (4) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene),
   or a salt thereof.

### Compound (B1)

A compound of the formula (I) wherein

ring A is a 4- to 7-membered monocyclic nitrogen-containing non-aromatic heterocycle optionally having substituent(s) or a 6- to 12-membered fused nitrogen-containing non-aromatic heterocycle optionally having substituent(s),
R¹ is a hydrogen atom, C₁₋₆ alkyl optionally having substituent(s) or —OR¹¹ wherein R¹¹ is as defined above;
R² is a hydrogen atom or C₁₋₆ alkyl optionally having substituent(s);
R³ is a hydrogen atom, C₁₋₆ alkyl optionally having substituent(s) or C₃₋₈ cycloalkyl optionally having substituent(s); or,
R¹ and R³ in combination form, together with the adjacent nitrogen atom and carbon atom, a 4- to 7-membered nitrogen-containing non-aromatic heterocycle optionally having substituent(s);

### R⁴ is

(1) C₃₋₈ cycloalkyl optionally having substituent(s),
(2) a 4- to 7-membered monocyclic non-aromatic heterocyclic group optionally having substituent(s),
(3) C₁₋₆ alkyl optionally having substituent(s), or
(4) C₆₋₁₀ aryl optionally having substituent(s); and

### R⁵ is

(1) a 5- to 7-membered monocyclic aromatic heterocyclic group optionally having substituent(s),
(2) a 8- to 12-membered fused aromatic heterocyclic group optionally having substituent(s),
(3) —C(═O)NR⁵¹—X¹—R⁵² wherein R⁵¹, X¹ and R⁵² are as defined above,
(4) —X²—R⁵² wherein R⁵² and X² are as defined above, or
(5) —X²—NR⁵¹R⁵³ wherein R⁵¹, X² and R⁵³ are as defined above,
or a salt thereof.

### Compound (B2)

A compound of the formula (I) wherein

ring A is
(1) a 4- to 7-membered (preferably 5- or 6-membered) monocyclic nitrogen-containing non-aromatic heterocycle having “—CH₂—NH—” as a ring-constituting member (e.g., pyrrolidine) and optionally having 1 to 5 (preferably 1 to 3) substituents selected from
(a) a halogen atom (e.g., a fluorine atom);
(b) hydroxy;
(c) C₁₋₆ alkyl (e.g., methyl) optionally having 1 to 3 halogen atoms (e.g., a fluorine atom);
(d) C₁₋₆ alkoxy (e.g., methoxy, ethoxy) optionally having 1 to 3 substituents selected from
   (i) a halogen atom (e.g., a fluorine atom),
   (ii) C₁₋₆ alkoxy (e.g., ethoxy), and
   (iii) C₃₋₈ cycloalkyl (e.g., cyclopropyl); and
(e) oxo; or
   (2) a 6- to 12-membered fused nitrogen-containing non-aromatic heterocycle (e.g., azabicyclohexane (e.g., 2-azabicyclo[3.1.0]hexane));

### R¹ is

(1) a hydrogen atom; or
(2) C₁₋₆ alkyl (e.g., methyl, ethyl) optionally having 1 to 3 substituents selected from
(a) amino,
(b) imino, and
(c) C₃₋₈ cycloalkyl (e.g., cyclopropyl);
   R² is a hydrogen atom or C₁₋₆ alkyl (e.g., methyl);

### R³ is

(1) a hydrogen atom;
(2) C₁₋₆ alkyl (e.g., methyl, ethyl, isopropyl) optionally having 1 to 3 substituents selected from
(a) a halogen atom (e.g., a fluorine atom),
(b) hydroxy,
(c) cyano, and
(d) C₃₋₈ cycloalkyl (e.g., cyclopropyl); and
   (3) C₃₋₈ cycloalkyl (e.g., cyclopropyl); or,
   R¹ and R³ in combination form, together with the adjacent nitrogen atom and carbon atom, a 4- to 7-membered (preferably 4 to 6-membered) nitrogen-containing non-aromatic heterocycle azetidine, pyrrolidine);

### R⁴ is

(1) C₃₋₈ cycloalkyl (e.g., cyclohexyl) optionally having 1 to 3 halogen atoms (e.g., a fluorine atom);
(2) a 4- to 7-membered monocyclic non-aromatic heterocyclic group (e.g., tetrahydropyranyl);
(3) C₁₋₆ alkyl (e.g., propyl, butyl) having one amino having one substituent selected from
(a) C₁₋₆ alkyl-carbonyl (e.g., acetyl, ethylcarbonyl) optionally having one C₆₋₁₀ aryl (e.g., phenyl),
(b) C₁₋₆ alkoxy-carbonyl (e.g., methoxycarbonyl) optionally having one C₆₋₁₀ aryl (e.g., phenyl), and
(c) arylsulfonyl (e.g., phenylsulfonyl); or
   (4) C₆₋₁₀ aryl (e.g., phenyl, naphthyl) optionally having 1 to 3 halogen atoms (e.g., a fluorine atom); and

### R⁵ is

(1) a 5- to 7-membered monocyclic aromatic heterocyclic group (e.g., thiazolyl, oxazolyl, pyrazolyl, oxadiazolyl) optionally having one substituent selected from
(a) C₆₋₁₀ aryl (e.g., phenyl) optionally having 1 to 3 halogen atoms (e.g., a fluorine atom),
(b) C₆₋₁₀ aryl-carbonyl (e.g., benzoyl, 1-naphthoyl) optionally having 1 to 3 substituents selected from a halogen atom (e.g., a fluorine atom), C₁₋₆ alkyl (e.g., methyl) optionally having 1 to 3 halogen atoms (e.g., a fluorine atom), C₁₋₆ alkoxy (e.g., methoxy, 2-methylpropoxy) and C₆₋₁₀ aryl (e.g., phenyl),
(c) 5- to 7-membered monocyclic aromatic heterocyclyl-carbonyl (e.g., thienylcarbonyl, furylcarbonyl, pyrazolylcarbonyl, isooxazolylcarbonyl, pyrimidinylcarbonyl) optionally having 1 to 3 substituents selected from cyano and C₁₋₆ alkyl (e.g., methyl),
(d) 8- to 12-membered fused aromatic heterocyclyl-carbonyl (e.g., benzothienylcarbonyl),
(e) C₇₋₁₃ aralkyl (e.g., benzyl) optionally having 1 to 3 halogen atoms (e.g., a fluorine atom), and
(f) C₂₋₆ alkenyl-carbonyl (e.g., vinylcarbonyl, allylcarbonyl) optionally having one C₆₋₁₀ aryl (e.g., phenyl);
   (2) —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring (e.g., 1,2,3,4-tetrahydronaphthyl, 2,3-dihydroindenyl, fluorenyl), which optionally has 1 to 3 substituents selected from
   (i) hydroxy,
   (ii) a halogen atom (e.g., a fluorine atom),
   (iii) C₁₋₆ alkyl-carbonyloxy (e.g., acetyloxy), and
   (iv) oxo;
(b) a monovalent group derived from a fused ring formed by condensation of tetrahydropyran or tetrahydrofuran and a benzene ring (e.g., 2,3-dihydrochromenyl, 2,3-dihydrobenzofuranyl), which optionally has 1 to 3 halogen atoms (e.g., a fluorine atom);
(c) C₆₋₁₀ aryl (e.g., phenyl, naphthyl) optionally having 1 to 3 C₁₋₆ alkyl (e.g., methyl);
(d) a 5- to 7-membered monocyclic aromatic heterocyclic group (e.g., thiadiazolyl) optionally having one C₆₋₁₀ aryl (e.g., phenyl); or
(e) dihydroindolyl, and
X¹ is
(a) a bond, or
(b) C₁₋₆ alkylene (e.g., —CH₂—, —CH(CH₃)—C(CH₃)₂—) optionally having 1 to 3 substituents selected from (i) C₆₋₁₀ aryl (e.g., phenyl) and (ii) a halogen atom (e.g., a fluorine atom);
   (3) —X²—R⁵² wherein
R⁵² is a 8- to 12-membered fused non-aromatic heterocyclic group (e.g., dihydroindolyl), and
X² is C₁₋₆ alkylene (e.g., methylene); or
   (4) —X²—NR⁵¹R⁵³ wherein
R⁵¹ is a hydrogen atom,
R⁵³ is a monovalent group derived from a fused ring formed by condensation of C₃₋₈ cycloalkane and a benzene ring (e.g., 1,2,3,4-tetrahydronaphthyl), and
X² is C₁₋₆ alkylene (e.g., methylene), or a salt thereof.

### Compound (33)

Compound (B2) wherein

ring A is
(1) a 4- to 7-membered (preferably 5- or 6-membered) monocyclic nitrogen-containing non-aromatic heterocycle (e.g., pyrrolidine) and optionally having 1 to 5 (preferably 1 to 3) substituents selected from
(a) a halogen atom (e.g., a fluorine atom);
(b) hydroxy;
(c) C₁₋₆ alkyl (e.g., methyl) optionally having 1 to 3 halogen atoms (e.g., a fluorine atom);
(d) C₁₋₆ alkoxy (e.g., methoxy, ethoxy) optionally having 1 to 3 substituents selected from
   (i) a halogen atom (e.g., a fluorine atom),
   (ii) C₁₋₆ alkoxy (e.g., ethoxy), and
   (iii) C₃₋₈ cycloalkyl (e.g., cyclopropyl); and
(e) oxo,
   or a salt thereof.

### Compound (B4)

Compound (B3) wherein

R⁵ is —C(═O)NR⁵¹—X¹—R⁵² wherein
R⁵¹ is a hydrogen atom,
R⁵² is a monovalent group derived from a fused ring formed by condensation of tetrahydropyran and a benzene ring (e.g., 2,3-dihydrochromenyl), which optionally has 1 to 3 halogen atoms (e.g., a fluorine atom), and
X¹ is a bond,
   or a salt thereof.

### Compound (D)

(3S,7R,8aR)-2-{(2S)-2-Cyclohexyl-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide (Example 18);
(3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide (Example 19);
(3S,7R,8aR)—N-[(4R)-3,4-dihydro-2H-chromen-4-yl]-7-ethoxy-2-{(2S)-2-[(N-methyl-L-alanyl)amino]-2-phenylacetyl}octahydropyrrolo[1,2-a]pyrazine-3-carboxamide (Example 21);
(3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide (Example 23);
(3S,8aR)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide (Example 29); or
(3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide (Example 32);
   or a salt thereof.

When compound (I) is the form of a salt, examples of such salt include metal salts, ammonium salts, salts with organic bases, salts with inorganic acids, salts with organic acids, salts with basic or acidic amino acids and the like. Preferable examples of the metal salt include alkali metal salts such as sodium salt, potassium salt and the like; alkaline earth metal salts such as calcium salt, magnesium salt, barium salt and the like; aluminum salts and the like. Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine or N,N′-dibenzylethylenediamine. Preferable examples of the salt with inorganic acid include a salt with hydrochloric acid, hydrobromic acid, nitric acid, sulfuric acid or phosphoric acid. Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid or p-toluenesulfonic acid. Preferable examples of the salt with basic amino acid include a salt with arginine, lysine or ornithine. Preferable examples of the salt with acidic amino acid include a salt with aspartic acid or glutamic acid.

Of these, pharmaceutically acceptable salt is preferable. When compound (I) has an acidic functional group, preferable examples thereof include inorganic salts such as an alkali metal salt (e.g., sodium salt, potassium salt), an alkaline earth metal salt (e.g., calcium salt, magnesium salt) and the like, and ammonium salts. When compound (I) has an basic functional group, preferable examples thereof include salts with inorganic acid such as hydrochloric acid, hydrobromic acid, nitric acid, sulfuric acid, phosphoric acid and the like; and salts with organic acid such as acetic acid, phthalic acid, fumaric acid, oxalic acid, tartaric acid, maleic acid, citric acid, succinic acid, methanesulfonic acid, benzenesulfonic acid, p-toluenesulfonic acid and the like.

Compound (I) may be a pharmaceutically acceptable cocrystal or a cocrystal salt. Here, the cocrystal or cocrystal salt means a crystalline substance, which is constituted from two or more kinds of specific solids each having different physical properties (e.g., structure, melting point, heat of fusion, hygroscopicity, stability etc.) at room temperature. The cocrystal and cocrystal salt can be produced according to a cocrystallization method known per se.

The production method of the compound of the present invention is explained in the following.

The compound of the present invention can be produced according to a method known per se, for example, the method described in the following, or a method analogous thereto.

Examples of the alcohol to be used as a reaction solvent for the following reaction include methanol, ethanol, propanol, isopropanol, butanol and tert-butanol.

Examples of the ether to be used as a reaction solvent for the following reaction include dioxane, tetrahydrofuran, diethyl ether, tert-butyl methyl ether, diisopropyl ether and ethylene glycol-dimethyl ether.

Examples of the ester to be used as a reaction solvent for the following reaction include ethyl formate, ethyl acetate and n-butyl acetate.

Examples of the halogenated hydrocarbon to be used as a reaction solvent for the following reaction include dichloromethane, chloroform and carbon tetrachloride, trichloroethylene.

Examples of the hydrocarbon to be used as a reaction solvent for the following reaction include n-hexane, benzene and toluene.

Examples of the amide to be used as a reaction solvent for the following reaction include formamide, N,N-dimethylformamide and N,N-dimethylacetamide.

Examples of the nitrile to be used as a reaction solvent for the following reaction include acetonitrile and propionitrile.

Examples of the sulfoxide to be used as a reaction solvent for the following reaction include dimethyl sulfoxide.

Examples of the aromatic hydrocarbon to be used as a reaction solvent for the following reaction include benzene and toluene.

Examples of the base to be used for the following reaction include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal C₁₋₆ alkoxides such as sodium methoxide, sodium ethoxide, potassium tert-butoxide and the like; organic bases such as trimethylamine, triethylamine, diisopropylethylamine, pyridine, picoline, N-methylpyrrolidine, N-methylmorpholine, N,N-dimethylaniline, 1,5-diazabicyclo[4.3.0]-5-nonene, 1,4-diazabicyclo[2.2.2]octane, 1,8-diazabicyclo[5.4.0]-7-undecene, tetramethylguanidine and the like; organic lithiums such as methyllithium, n-butyllithium, sec-butyllithium, tert-butyllithium and the like; and lithium amides such as lithiumdiisopropylamide and the like.

Examples of the acid to be used for the following reaction include inorganic acids such as hydrochloric acid, hydrobromic acid, sulfuric acid, phosphoric acid, perchloric acid and the like; sulfonic acids such as methanesulfonic acid, ethanesulfonic acid, benzenesulfonic acid, toluenesulfonic acid, camphorsulfonic acid and the like; and organic acids such as formic acid, acetic acid, propionic acid, trifluoroacetic acid and the like.

Examples of the alkali metal to be used for the following reaction include lithium, sodium, potassium, cesium and the like.

Examples of the alkaline earth metal to be used for the following reaction include magnesium, calcium and the like.

Examples of the alkali metal hydroxide to be used for the following reaction include lithium hydroxide, sodium hydroxide, potassium hydroxide, hydroxide cesium and the like.

Examples of the alkaline earth metal hydroxide to be used for the following reaction include magnesium hydroxide, calcium hydroxide and the like.

In the following reaction, starting material compound and production intermediate may be in the form of a salt. Examples of the salt include salts similar to the salts of compound (I).

The compound obtained in each step can also be used for the next reaction directly as the reaction mixture or as a crude product. In addition, it can also be isolated from the reaction mixture according to a conventional method (e.g., separation means such as recrystallization, distillation, chromatography and the like).

The production method of compound (I) is explained in the following.

Compound (I) can be produced, for example, according to the method shown in the following Reaction Scheme 1 or an analogous method thereto.

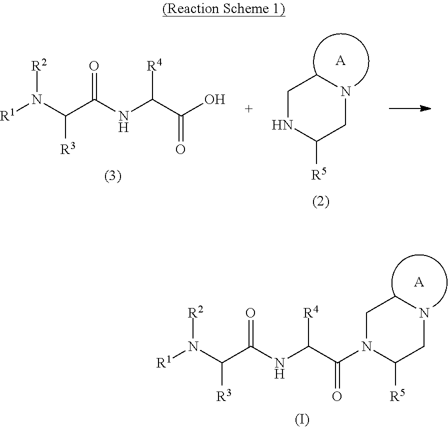

wherein each symbol is as defined above.

Compound (I) can be produced by reacting compound (2) with compound (3).

The amount of compound (3) to be used is generally 1 to 1000 equivalents, preferably 1 to 20 equivalents, per 1 equivalent of compound (2).

When compound (3) is used in the form of a free acid or a salt for the reaction, the reaction is desirably carried out in the presence of a condensing agent.

Examples of the condensing agent include carbodiimides such as N,N′-dicyclohexylcarbodiimide, N-cyclohexyl-N′-morpholinoethylcarbodiimide, N-cyclohexyl-N′-(4-diethylaminocyclohexyl)carbodiimide, N,N′-diethylcarbodiimide, N,N′-diisopropylcarbodiimide, N-ethyl-N′-(3-dimethylaminopropyl)carbodiimide and the like; triazines such as 2-chloro-4,6-dimethoxy-1,3,5-triazine, 4-(4,6-dimethoxy-1,3,5-triazin-2-yl)-4-methylmorpholinium chloride and the like; N,N′-carbonylbis(2-methylimidazole); trialkyl phosphite; polyphosphates such as ethyl polyphosphate, isopropyl polyphosphate and the like; phosphorus oxychloride; diphenylphosphoryl azide; thionyl chloride; oxalyl chloride; lower alkyl haloformates such as ethyl chloroformate, isopropyl chloroformate and the like; triphenylphosphine; 1-[bis(dimethylamino)methylene]-1H-1,2,3-triazolo(4,5-b)pyridinium 3-oxide hexafluorophosphate (HATU); N-hydroxybenzotriazole; 1-(p-chlorobenzenesulfonyloxy)-6-chloro-1H-benzotriazole; Vilsmeier reagent, which is prepared by reacting N,N′-dimethylformamide and thionyl chloride, phosgene, trichloromethyl chloroformate, phosphorus oxychloride or the like; and the like, and a combination thereof.

The amount of the condensing agent to be used is generally 1 to 1000 equivalents, preferably 1 to 20 equivalents, per 1 equivalent of compound (2).

This reaction may be carried out in the presence of a base, if desired.

The amount of the base to be used is generally 1 to 1000 equivalents, preferably 1 to 20 equivalents, per 1 equivalent of compound (2).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (3). For example, compound (3) is reacted with an alkyl chlorocarbonate (e.g., methyl chlorocarbonate, ethyl chlorocarbonate, isobutyl chlorocarbonate) in the presence of a base, and then the obtained mixed anhydride is reacted with compound (2).

The amount of the alkyl chlorocarbonate to be used is generally 1 to 1000 equivalents, preferably 1 to 20 equivalents, per 1 equivalent of compound (2).

The amount of the base to be used is generally 1 to 1000 equivalents, preferably 1 to 20 equivalents, per 1 equivalent of compound (2).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (2) can be synthesized according to a method known per se (e.g., the methods described in Bioorg. Med. Chem., 2006, 14, 6586-6592; Bioorg. Med. Chem. Lett. 2006, 16, 5462-5467; Tetrahedron: Asymmetry, 1996, 7, 1999-2005; Tetrahedron: Asymmetry, 1993, 4, 2389-2398; WO 2007/28654; U.S. Pat. No. 4,400,511 and the like; the methods described in the below-mentioned Reaction Schemes 2 to 7).

Compound (3) can be synthesized according to a method known per se (e.g., the method described in the below-mentioned Reaction Scheme 9).

Compound (2a) which is compound (2) wherein R⁵ is —C(═O)—N(R⁵¹)—R⁵² can be produced, for example, according to the method shown in the following Reaction Scheme 2.

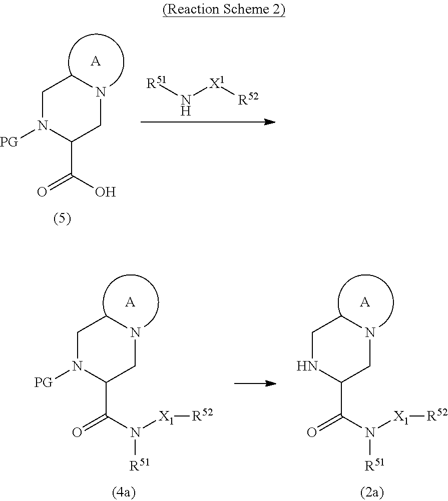

wherein PG is an amino-protecting group, and the other symbols are each as defined above.

Examples of the amino-protecting group for PG include formyl, C₁₋₆ alkyl-carbonyl, C₁₋₆ alkoxy-carbonyl, benzoyl, C₇₋₁₀ aralkyl-carbonyl (e.g., benzylcarbonyl), C₇₋₁₄ aralkyloxy-carbonyl (e.g., benzyloxycarbonyl, 9-fluorenylmethoxycarbonyl), benzyl, trityl, phthaloyl, N,N-dimethylaminomethylene, substituted silyl (e.g., trimethylsilyl, triethylsilyl, dimethylphenylsilyl, tert-butyldimethylsilyl, tert-butyldiethylsilyl) and C₂₋₆ alkenyl (e.g., 1-allyl). These groups are optionally substituted by 1 to 3 substituents selected from a halogen atom, C₁₋₆ alkoxy and nitro.

Compound (4a) can be produced by reacting compound (5) with an amine represented by HN(R⁵¹)—X¹—R⁵².

The amount of compound (5) to be used is generally 1 to 1000 equivalents, preferably 1 to 20 equivalents, per 1 equivalent of the above-mentioned amine.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of the above-mentioned amine.

The reaction may be carried out in the presence of a base in the same manner as in Reaction Scheme 1, if desired.

The amount of the base to be used is generally 1 to 1000 equivalents, preferably 1 to 20 equivalents, per 1 equivalent of the above-mentioned amine.

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with an amine represented by HN(R⁵¹)—X¹—R⁵².

The amount of the alkyl chlorocarbonate to be used is generally 1 to 1000 equivalents, preferably 1 to 20 equivalents, per 1 equivalent of the above-mentioned amine.

The amount of the base to be used is generally 1 to 1000 equivalents, preferably 1 to 20 equivalents, per 1 equivalent of the above-mentioned amine.

The reaction temperature for this reaction is generally −78° C. to 200° C. The reaction time is generally 0.5 to 100 hr.

Compound (5) can be synthesized according to a method known per se (e.g., the method described in the below-mentioned Reaction Scheme 8 or 19).

The amine represented by HN(R⁵¹)—X¹—R⁵² may be commercially available, or can be synthesized according to a method known per se (e.g., the methods described in Tetrahedron: Asymmetry, 1997, 8, 895-902; Chem. Eur. J., 2005, 11, 5777-5785; J. Org. Chem., 2006, 71, 6859-6862; US 2007/0219237; US 2004/0157739; WO 2008/039640; WO 2009/048474 and the like).

Compound (2a) can be produced by removing the PG of compound (4a).

The removal of the protecting group represented by PG can be carried out according to a method known per se, for example, the methods described in Protective Groups in Organic Synthesis, John Wiley and Sons (1980) and the like, specifically, by a method using an acid, a base, ultraviolet rays, hydrazine, phenylhydrazine, sodium N-methyldithiocarbamate, tetrabutylammonium fluoride, palladium acetate, trialkylsilylhalide (e.g., trimethylsilyliodide, trimethylsilylbromide) and the like, a reduction method and the like.

Compound (2b) which is compound (2) wherein R⁵ is —CH₂—NR⁵¹R⁵³ can be produced, for example, according to the method shown in the following Reaction Scheme 3.

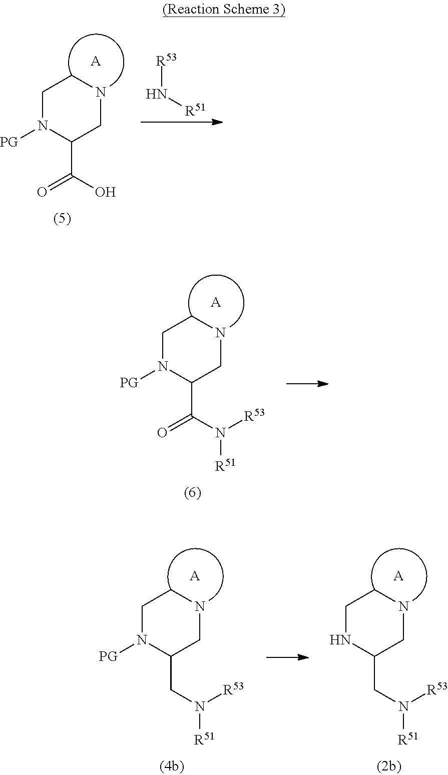

wherein each symbol is as defined above.

Compound (6) can be produced by reacting compound (5) with an amine represented by HNR⁵¹R⁵³.

The amount of compound (5) to be used is generally 1 to 1000 equivalents, preferably 1 to 20 equivalents, per 1 equivalent of the above-mentioned amine.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of the above-mentioned amine.

The reaction may be carried out in the presence of a base in the same manner as in Reaction Scheme 1, if desired.

The amount of the base to be used is generally 1 to 1000 equivalents, preferably 1 to 20 equivalents, per 1 equivalent of the above-mentioned amine.

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with an amine represented by HNR⁵¹R⁵³.

The amount of the alkyl chlorocarbonate to be used is generally 1 to 1000 equivalents, preferably 1 to 20 equivalents, per 1 equivalent of the above-mentioned amine.

The amount of the base to be used is generally 1 to 1000 equivalents, preferably 1 to 20 equivalents, per 1 equivalent of the above-mentioned amine.

The reaction temperature for this reaction is generally −78° C. to 200° C. The reaction time is generally 0.5 to 100 hr.

Compound (4b) can be produced by subjecting compound (6) to a reduction reaction known'per se (e.g., the method described in Tetrahedron: Asymmetry, 2006, 17, 491-493) to convert the carbonyl of compound (6) into methylene.

The reduction reaction is carried out, for example, using a reducing agent. Examples of the reducing agent include lithium aluminum hydride, borane-ether complex, borane-tetrahydrofuran complex and borane-dimethylsulfide complex.

The amount of the reducing agent to be used is generally 1 to 1000 equivalents, preferably 1 to 20 equivalents, per 1 equivalent of compound (6).

This reaction is preferably carried out in a solvent inert to the reaction. The solvent is not particularly limited as long as the reaction proceeds, and examples thereof include ethers.

The reaction temperature for this reaction is generally −78° C. to 200° C. The reaction time is generally 0.5 to 100 hr.

Compound (2b) can be produced by removing the PG of compound (4b) in the same manner as in the production method of compound (2a) from compound (4a) in Reaction Scheme 2.

The amine represented by HNR⁵¹R⁵³ may be commercially available, or can be synthesized according to a method known per se.

Compound (2c) which is compound (2) wherein R⁵ is —CH₂—NR⁵¹—C(═O)—R⁵³ can be produced, for example, according to the method shown in the following Reaction Scheme 4.

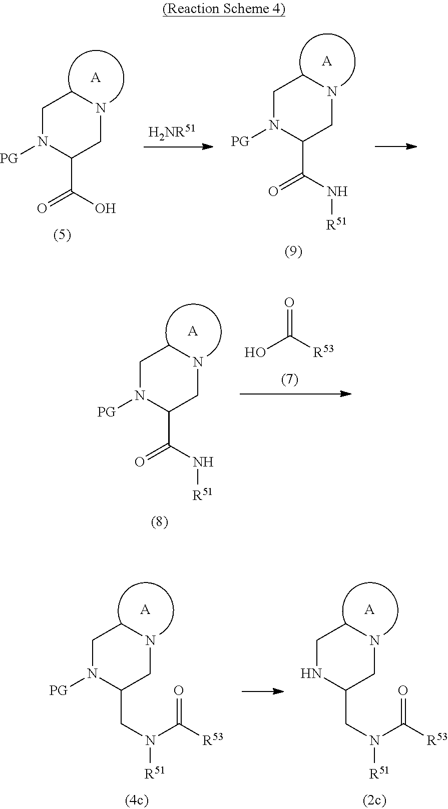

wherein each symbol is as defined above.

Compound (9) can be produced by reacting compound (5) with an amine represented by H₂NR⁵¹.

The amount of compound (5) to be used is generally 1 to 1000 equivalents, preferably 1 to 20 equivalents, per 1 equivalent of the above-mentioned amine.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of the above-mentioned amine.

The reaction may be carried out in the presence of a base in the same manner as in Reaction Scheme 1, if desired.

The amount of the base to be used is generally 1 to 1000% equivalents, preferably 1 to 20 equivalents, per 1 equivalent of the above-mentioned amine.

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with an amine represented by H₂NR⁵¹.

The amount of the alkyl chlorocarbonate to be used is generally 1 to 1000 equivalents, preferably 1 to 20 equivalents, per 1 equivalent of the above-mentioned amine.

The amount of the base to be used is generally 1 to 1000 equivalents, preferably 1 to 20 equivalents, per 1 equivalent of the above-mentioned amine.

The reaction temperature for this reaction is generally −78° C. to 200° C. The reaction time is generally 0.5 to 100 hr.

Compound (8) can be produced by subjecting compound (9) to a reduction reaction in the same manner as in the production method of compound (4b) from compound (6) in Reaction Scheme 3 to convert the carbonyl of compound (9) into methylene.

The amount of the reducing agent to be used is generally 1 to 1000 equivalents, preferably 1 to 20 equivalents, per 1 equivalent of compound (9).

Compound (4c) can be produced by reacting compound (8) with compound (7).

The amount of compound (7) to be used is generally 1 to 1000 equivalents, preferably 1 to 20 equivalents, per 1 equivalent of compound (8).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (8).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (8).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (7). For example, compound (7) is reacted with an alkyl chlorocarbonate in the presence of a base in the same manner as in Reaction Scheme 1, and then the obtained mixed anhydride is reacted with compound (8).

The amount of the alkyl chlorocarbonate to be used is generally 1 to 1000 equivalents, preferably 1 to 20 equivalents, per 1 equivalent of compound (8).

The amount of the base to be used is generally 1 to 1000 equivalents, preferably 1 to 20 equivalents, per 1 equivalent of compound (8).

The reaction temperature for this reaction is generally −78° C. to 200° C. The reaction time is generally 0.5 to 100 hr.

Compound (2c) can be produced by removing the PG of compound (4c) in the same manner as in the production method of compound (2a) from compound (4a) in Reaction Scheme 2.

The amine represented by H₂NR⁵¹ may be commercially available, or can be synthesized according to a method known per se.

Compound (7) may be commercially available, or can be synthesized according to a method known per se.

Compound (2d) which is encompassed in compound (2) can be produced, for example, according to the method shown in the following Reaction Scheme 5.

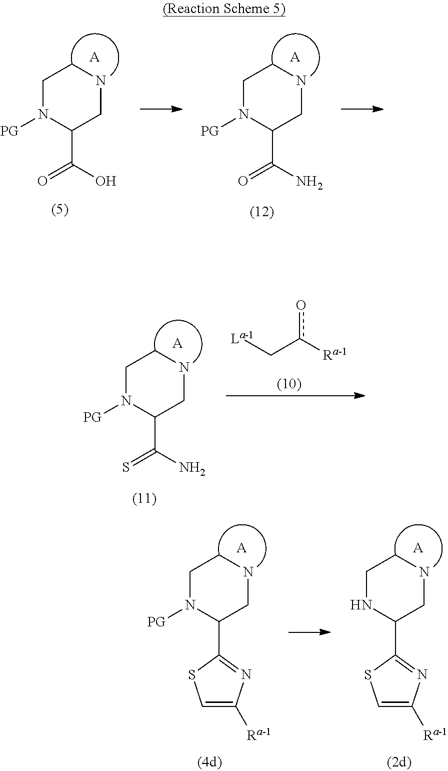

wherein L^{a-1} is a leaving group, R^{a-1} is a substituent, and the other symbols are each as defined above.

Examples of the leaving group for L^{a-1} include a halogen atom (e.g., fluorine, chlorine, bromine, iodine), a group represented by the formula —O—S(O)ₖR^{aa}, and a group represented by the formula —OR^{aa}, wherein k is an integer of 0, 1 or 2, and R^{aa} is C₁₋₄ alkyl (e.g., methyl, ethyl, propyl), benzyl or C₆₋₁₀ aryl (e.g., phenyl, tolyl).

Examples of the substituent for R^{a-1} include substituents selected from the above-mentioned Substituent Group B.

Compound (12) can be produced by reacting compound (5) with ammonia or an ammonium salt represented by the formula X⁻NH₄⁺ wherein X⁻ is a halogen ion (e.g., chlorine ion, bromine ion, iodine ion) or an organic acid residue (e.g., HCOO⁻).

The amount of the ammonia or ammonium salt to be used is generally 1 to 1000 equivalents, preferably 1 to 20 equivalents, per 1 equivalent of compound (5).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (5).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (5).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with ammonia or an ammonium salt.

The amount of the alkyl chlorocarbonate to be used is generally 1 to 1000 equivalents, preferably 1 to 20 equivalents, per 1 equivalent of compound (5).

The amount of the base to be used is generally 1 to 1000 equivalents, preferably 1 to 20 equivalents, per 1 equivalent of compound (5).

The reaction temperature for this reaction is generally −78° C. to 200° C. The reaction time is generally 0.5 to 100 hr.

Compound (11) can be produced by reacting compound (12) with a thionating agent to convert the carbonyl of compound (12) into thiocarbonyl.

Examples of the thionating agent include Lawesson reagent, diphosphorus pentasulfide and the like.

The amount of the thionating agent to be used is generally 1 to 1000 equivalents, preferably 1 to 20 equivalents, per 1 equivalent of compound (12).

This reaction is preferably carried out in a solvent inert to the reaction. The solvent is not particularly limited as long as the reaction proceeds, and examples thereof include halogenated hydrocarbons, aromatic hydrocarbons, ethers and the like. These solvents may be used in a mixture of two or more kinds thereof at an appropriate ratio.

The reaction temperature for this reaction is generally −78° C. to 200° C. The reaction time is generally 0.5 to 100 hr.

Compound (4d) can be produced by reacting compound (11) with compound (10).

The amount of compound (10) to be used is generally 1 to 1000 equivalents, preferably 1 to 20 equivalents, per 1 equivalent of compound (11).

This reaction is preferably carried out in a solvent inert to the reaction. The solvent is not particularly limited as long as the reaction proceeds, and examples thereof include ethers, halogenated hydrocarbons, hydrocarbons, nitriles and sulfoxides. These solvents may be used in a mixture of two or more kinds thereof at an appropriate ratio.

The reaction temperature for this reaction is generally −78° C. to 200° C. The reaction time is generally 0.5 to 100 hr.

Compound (2d) can be produced by removing the PG of compound (4d) in the same manner as in the production method of compound (2a) from compound (4a) in Reaction Scheme 2.

The ammonium salt represented by X⁻NH₄⁺ may be commercially available, or can be produced by reacting an acid represented by XH with ammonia according to a method known per se.

Compound (10) may be commercially available, or can be synthesized according to a method known per se (e.g., the method described in J. Org. Chem. 1983, 48, 2520-2527).

Compound (2e) which is encompassed in compound (2) can be produced, for example, according to the method shown in the following Reaction Scheme 6.

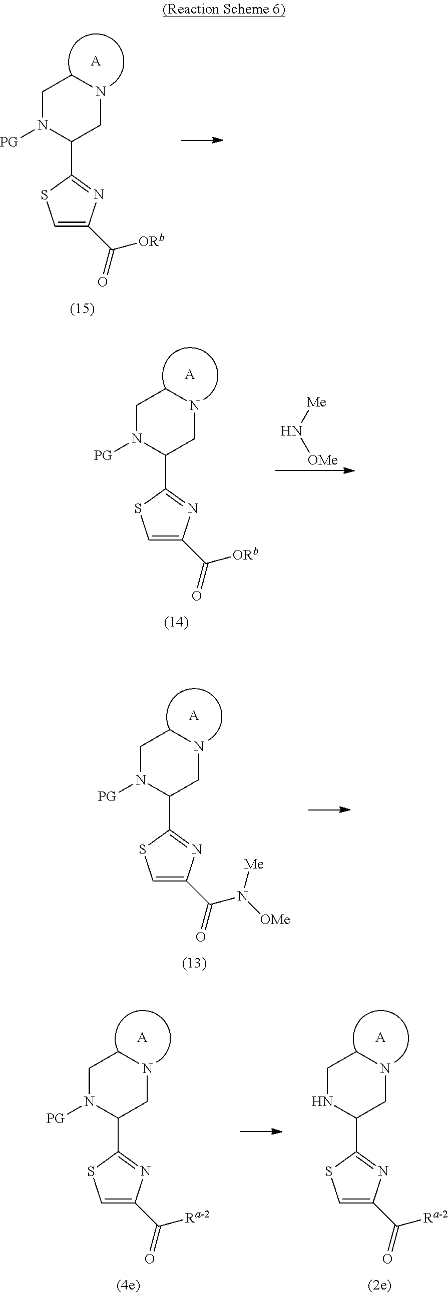

wherein
R^{b} is C₁₋₆ alkyl optionally having 1 to 3 substituents selected from
(a) a halogen atom,
(b) hydroxy,
(c) carboxy,
(d) C₁₋₆ alkoxy,
(e) C₁₋₆ alkoxy-carbonyl, and
(f) amino optionally having 1 or 2 C₁₋₆ alkyl;

### R^{a-2} is

(1) C₁₋₆ alkyl optionally having 1 to 3 halogen atoms,
(2) C₆₋₁₀ aryl optionally having 1 to 3 substituents selected from
(a) a halogen atom, and
(b) C₁₋₆ alkyl optionally having 1 to 3 halogen atoms,
   (3) a 5- to 12-membered aromatic heterocyclic group optionally having 1 to 3 substituents selected from
(a) a halogen atom, and
(b) C₁₋₆ alkyl optionally having 1 to 3 halogen atoms, or
   (4) a 4- to 12-membered non-aromatic heterocyclic group optionally having 1 to 3 substituents selected from
(a) a halogen atom, and
(b) C₁₋₆ alkyl optionally having 1 to 3 halogen atoms, and other symbols are each as defined above.

Compound (14) can be produced by subjecting compound (15) to a method known per se for conversion into carboxylic acid (e.g., the method described in Comprehensive Organic Transformations, John Wiley and Sons (1999)).

Specifically, compound (14) can be produced by reacting compound (15) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (15).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (13) (i.e., Weinreb amide) can be produced by reacting compound (14) with N,O-dimethylhydroxylamine (or a salt thereof).

The amount of the N,O-dimethylhydroxylamine (or a salt thereof) to be used is generally 1 to 1000 equivalents, preferably 1 to 20 equivalents, per 1 equivalent of compound (14).

N,O-Dimethylhydroxylamine (or a salt thereof) may be commercially available, or can be produced according to a method known per se.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (14).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (14).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (14). For example, compound (14) is reacted with an alkyl chlorocarbonate in the presence of a base in the same manner as in Reaction Scheme 1, and then the obtained mixed anhydride is reacted with N,O-dimethylhydroxylamine (or a salt thereof).

The amount of the alkyl chlorocarbonate to be used is generally 1 to 1000 equivalents, preferably 1 to 20 equivalents, per 1 equivalent of compound (14).

The amount of the base to be used is generally 1 to 1000 equivalents, preferably 1 to 20 equivalents, per 1 equivalent of compound (14).

The reaction temperature for this reaction is generally −78° C. to 200° C. The reaction time is generally 0.5 to 100 hr.

Compound (4e) can be produced by reacting compound (13) with a reagent corresponding to R^{a-2} according to a method known per se (e.g., the method described in Tetrahedron Letters, 1981, 22, 3815-3818).

Examples of the reagent include a magnesium reagent corresponding to R^{a-2}, and a lithium reagent corresponding to R^{a-2}.

The magnesium reagent corresponding to R^{a-2} may be commercially available as a Grignard reagent, or can be synthesized according to a method known per se (e.g., the method described in Heterocycles, 1987, 26, 3141-3151).

The lithium reagent corresponding to R^{a-2} may be commercially available, or can be synthesized according to a method known per se (e.g., the method described in J. Org. Chem., 1991, 56, 3750-3752).

The amount of the reagent corresponding to R^{a-2} to be used is generally 1 to 1000 equivalents, preferably 1 to 20 equivalents, per 1 equivalent of compound (13).

This reaction is preferably carried out in a solvent inert to the reaction. The solvent is not particularly limited as long as the reaction proceeds, and examples thereof include ethers and hydrocarbons. These solvents may be used in a mixture of two or more kinds thereof at an appropriate ratio.

The reaction temperature for this reaction is generally −78° C. to 200° C. The reaction time is generally 0.5 to 100 hr.

Compound (2e) can be produced by removing the PG of compound (4e) in the same manner as in the production method of compound (2a) from compound (4a) in Reaction Scheme 2.

Compound (15) can be produced using compound (10) wherein R^{a-1} is COOR^{b} in the same manner as in Reaction Scheme 5.

Compound (2f) which is encompassed in compound (2) can be produced, for example, according to the method shown in the following Reaction Scheme 7.

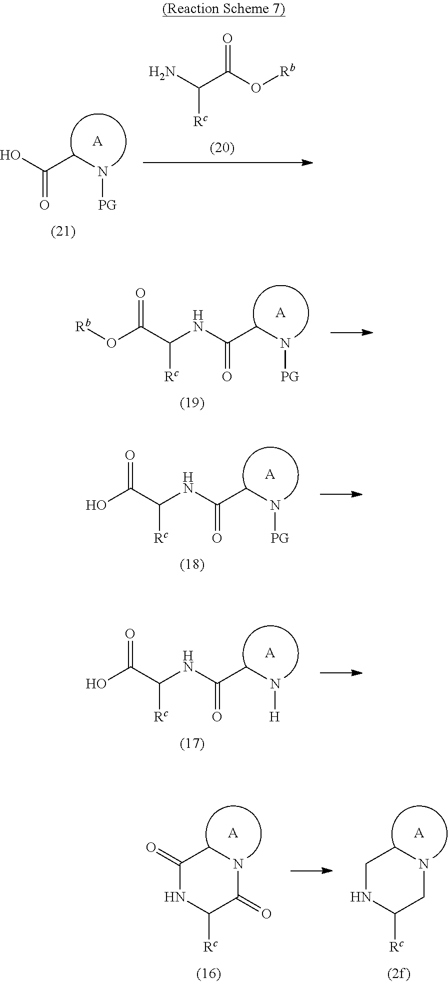

wherein R^{C} is a cyclic group optionally having substituent(s), —X²—R⁵², —X²—NR⁵¹R⁵³ or —X²—NR⁵¹—C(═O)—R⁵³, and the other symbols are each as defined above.

Compound (19) can be produced by reacting compound (20) with compound (21).

The amount of compound (21) to be used is generally 1 to 1000 equivalents, preferably 1 to 20 equivalents, per 1 equivalent of compound (20).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (20).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (20).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of'compound (21). For example, compound (21) is reacted with an alkyl chlorocarbonate in the presence of a base in the same manner as in Reaction Scheme 1, and then the obtained mixed anhydride is reacted with compound (20).

The amount of the alkyl chlorocarbonate to be used is generally 1 to 1000 equivalents, preferably 1 to 20 equivalents, per 1 equivalent of compound (21).

The amount of the base to be used is generally 1 to 1000 equivalents, preferably 1 to 20 equivalents, per 1 equivalent of compound (21).

The reaction temperature for this reaction is generally −78° C. to 200° C. The reaction time is generally 0.5 to 100 hr.

Compound (18) can be produced by converting compound (19) into the carboxylic acid in the same manner as in the production method of compound (14) from compound (15) in Reaction Scheme 6.

Specifically, compound (18) can be produced by reacting compound (19) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (19).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (17) can be produced by removing the PG of compound (18) in the same manner as in the production method of compound (2a) from compound (4a) in Reaction Scheme 2.

Compound (16) can be produced by subjecting compound (17) to an intramolecular condensation.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (17).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (17).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (2f) can be produced by subjecting compound (16) to a reduction reaction in the same manner as in the production method of compound (4b) from compound (6) in Reaction Scheme 3 to convert the carbonyl of compound (16) into methylene.

The amount of the reducing agent to be used is generally 1 to 1000 equivalents, preferably 1 to 20 equivalents, per 1 equivalent of compound (16).

The reaction temperature for this reaction is generally −78° C. to 200° C. The reaction time is generally 0.5 to 100 hr.

Compound (20) may be commercially available, or can be synthesized according to a method known per se (e.g., introduction and removal of protecting group according to the methods described in J. Heterocyclic Chem., 1991, 28, 1715-1720; WO 2008/85302; J. Am. Chem. Soc. 1995, 117, 9375-9376; J. Med. Chem., 1993, 36, 2300-2310; Tetrahedron: Asymmetry, 2001, 12, 2421-2425; WO 2009/005677; WO 2006/069063; US 2003/0216325 and the like; the method described in the below-mentioned Reaction Scheme 18 or 21; the method described in Protective Groups in Organic Synthesis, John Wiley and Sons (1980)). Compound (21) may be commercially available, or can be synthesized according to a method known per se.

Of compound (5), the following compound (5a) can be produced according to a method known per se (e.g., the method described in U.S. Pat. No. 4,400,511) or the method shown in the following Reaction Scheme 8.

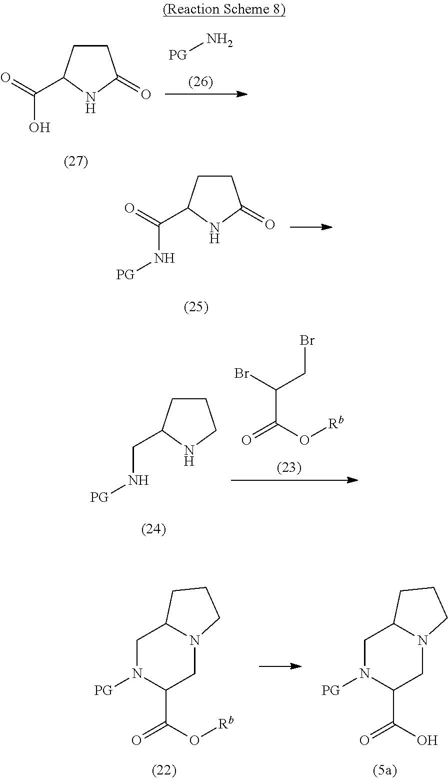

wherein each symbol is as defined above.

Compound (25) can be produced by reacting compound (26) with compound (27).

The amount of compound (27) to be used is generally 1 to 1000 equivalents, preferably 1 to 20 equivalents, per 1 equivalent of compound (26).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (26).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (26).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (27). For example, compound (27) is reacted with an alkyl chlorocarbonate in the presence of a base in the same manner as in Reaction Scheme 1, and then the obtained mixed anhydride is reacted with compound (26).

The amount of the alkyl chlorocarbonate to be used is generally 1 to 1000 equivalents, preferably 1 to 20 equivalents, per 1 equivalent of compound (26).

The amount of the base to be used is generally 1 to 1000 equivalents, preferably 1 to 20 equivalents, per 1 equivalent of compound (26).

The reaction temperature for this reaction is generally −78° C. to 200° C. The reaction time is generally 0.5 to 100 hr.

Compound (24) can be produced by subjecting compound (25) to a reduction reaction in the same manner as in the production method of compound (4b) from compound (6) in Reaction Scheme 3 to convert the carbonyl of compound (25) into methylene.

The amount of the reducing agent to be used is generally 2 to 1000 equivalents, preferably 2 to 20 equivalents, per 1 equivalent of compound (25).

The reaction temperature for this reaction is generally −78° C. to 200° C. The reaction time is generally 0.5 to 100 hr.

Compound (22) can be produced by reacting compound (24) with compound (23) in the presence of a base (e.g., triethylamine, N-methylmorpholine, N,N-dimethylaniline, sodium hydrogen carbonate, sodium carbonate, potassium carbonate).

The amount of compound (23) to be used is generally 1 to 1000 equivalents, preferably 1 to 20 equivalents, per 1 equivalent of compound (24).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The amount of the base to be used is generally 1 to 1000 equivalents, preferably 1 to 20 equivalents, per 1 equivalent of compound (24).

The reaction temperature for this reaction is generally −78° C. to 200° C. The reaction time is generally 0.5 to 100 hr.

Compound (5a) can be produced, by converting compound (22) into the carboxylic acid in the same manner as in the production method of compound (14) from compound (15) in Reaction Scheme 6.

Specifically, compound (5a) can be produced by reacting compound (22) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (22).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

In this reaction, the PG of compound (22) and the PG of compound (5a) are the same or different. When the PG of compound (22) and the PG of compound (5a) are different, the PG of compound (22) is removed according to a method known per se (e.g., the method described in Protective Groups in Organic Synthesis, John Wiley and Sons (1980)), and then the desired PG is introduced into compound (22) according to a method known per se (e.g., the method described in Protective Groups in Organic Synthesis, John Wiley and Sons (1980)).

Compound (26) may be commercially available, or can be synthesized according to a method known per se (e.g., the method described in Protective Groups in Organic Synthesis, John Wiley and Sons (1980) and the like).

Compound (27) may be commercially available.

Compound (23) may be commercially available, or can be synthesized according to a method known per se (e.g., the method described in J. Org. Chem., 1955, 20, 525-529).

Compound (3) can be produced, for example, according to the method shown in the following Reaction Scheme 9.

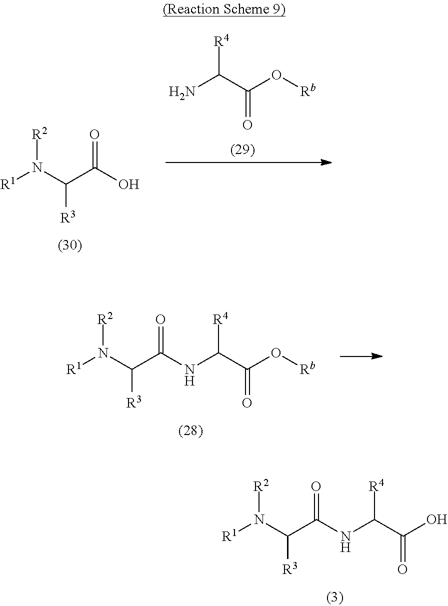

wherein each symbol is as defined above.

Compound (28) can be produced by reacting compound (29) with compound (30).

The amount of compound (30) to be used is generally 1 to 1000 equivalents, preferably 1 to 20 equivalents, per 1 equivalent of compound (29).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (29).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (29).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (30). For example, compound (30) is reacted with an alkyl chlorocarbonate in the presence of a base in the same manner as in Reaction Scheme 1, and then the obtained mixed anhydride is reacted with compound (29).

The amount of the alkyl chlorocarbonate to be used is generally 1 to 1000 equivalents, preferably 1 to 20 equivalents, per 1 equivalent of compound (29).

The amount of the base to be used is generally 1 to 1000 equivalents, preferably 1 to 20 equivalents, per 1 equivalent of compound (29).

The reaction temperature for this reaction is generally −78° C. to 200° C. The reaction time is generally 0.5 to 100 hr.

Compound (3) can be produced by converting compound (28) into the carboxylic acid in the same manner as in the production method of compound (14) from compound (15) in Reaction Scheme 6.

Specifically, compound (3) can be produced by reacting compound (28) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (28).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (29) may be commercially available, or can be synthesized according to a method known per se (e.g., the methods described in J. Heterocyclic Chem., 1991, 28, 1715-1720; WO 2008/85302; J. Am. Chem. Soc. 1995, 117, 9375-9376; J. Med. Chem., 1993, 36, 2300-2310; Tetrahedron: Asymmetry, 2001, 12, 2421-2425; the methods described in WO 2009/005677; WO 2006/069063; US 2003/0216325 and the like; the method described in the below-mentioned Reaction Scheme 18; the method for introduction and removal of protecting groups described in Protective Groups in Organic Synthesis, John Wiley and Sons (1980)).

Compound (30) may be commercially available, or can be synthesized according to a method known per se (e.g., the methods described in J. Heterocyclic Chem., 1991, 28, 1715-1720; WO 2008/85302; J. Am. Chem. Soc. 1995, 117, 9375-9376; J. Med. Chem., 1993, 36, 2300-2310; Tetrahedron: Asymmetry, 2001, 12, 2421-2425; U.S. Pat. No. 6,436,904; US 2009/123423; Eur. J. Org. Chem., 2003, 4757-4764; J. Org. Chem., 2003, 68, 7033-7040; WO 2009/005677; WO 2006/069063; US 2003/0216325 and the like; the method described in the below-mentioned Reaction Scheme 21; the method for introduction and removal of protecting groups described in Protective Groups in Organic Synthesis, John Wiley and Sons (1980)).

PG can be introduced into compound (30) at substitutable position according to a method known per se (e.g., the method described in Protective Groups in Organic Synthesis, John Wiley and Sons (1980)).

Compound (Ia) which is compound (I) wherein R² is a hydrogen atom can be produced, for example, according to the is method shown in the following Reaction Scheme 10 or an analogous method thereto.

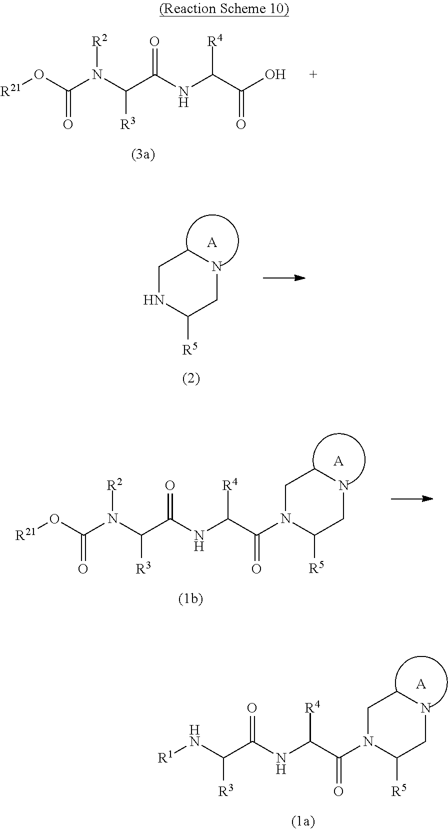

wherein each symbol is as defined above.

Compound (Ib) can be produced by reacting compound (2) with compound (3a).

The amount of compound (3a) to be used is generally 1 to 1000 equivalents, preferably 1 to 20 equivalents, per 1 equivalent of compound (2).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (2).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (2).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (3a). For example, compound (3a) is reacted with an alkyl chlorocarbonate in the presence of a base in the same manner as in Reaction Scheme 1, and then the obtained mixed anhydride is reacted with compound (2).

The amount of the alkyl chlorocarbonate to be used is generally 1 to 1000 equivalents, preferably 1 to 20 equivalents, per 1 equivalent of compound (2).

The amount of the base to be used is generally 1 to 1000 equivalents, preferably 1 to 20 equivalents, per 1 equivalent of compound (2).

The reaction temperature for this reaction is generally −78° C. to 200° C. The reaction time is generally 0.5 to 100 hr.

Compound (Ia) can be produced by converting the carbamate of compound (Ib) into the amine.

The conversion from the carbamate into the amine can be carried out according to a method known per se (e.g., the method described in Protective Groups in Organic Synthesis, John Wiley and Sons (1980)).

Specifically, a method using an acid, a base and the like, a reduction method and the like can be employed.

Compound (3a) can be synthesized according to a method known per se (e.g., the method shown in Reaction Scheme 9).

Compound (II) can be produced, for example, according to the method shown in the following Reaction Schemes 11 to 17.

Of compound (II), the following compound (IIa) can be produced, for example, according to the method shown in the following Reaction Scheme 11.

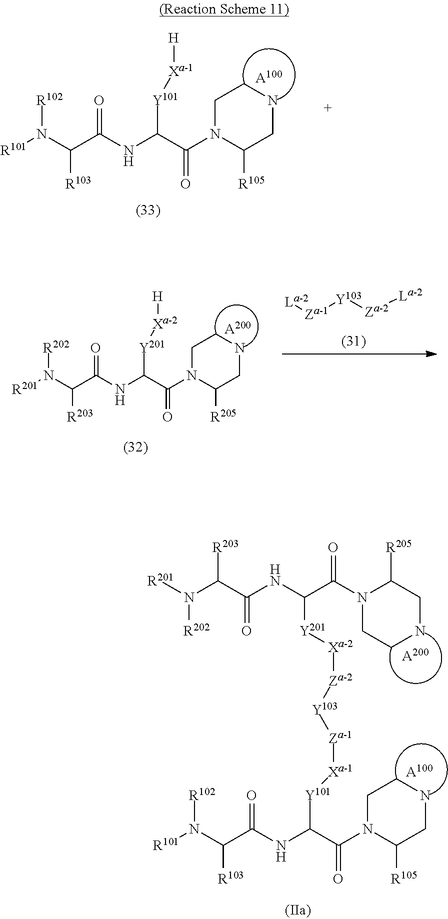

wherein X^{a-1} is a bond, —O—, —NR¹⁰⁶— or —S—, X^{a-2} is a bond, —O—, —NR²⁰⁶— or —S—, Z^{a-1} is a bond, —C(═O)—, —C(═O)—O—, —C(═O)—NR¹⁰⁶— or —S(═O)₂—, Z^{a-2} is a bond, —C(═O)—, —C(═O)—O—, —C(═O)—NR²⁰⁶— or —S(═O)₂—, L^{a-2} is a leaving group or hydroxy, and the other symbols are each as defined above.

Examples of the leaving group for L^{a-2} include those exemplified as the above-mentioned L^{a-1}.

Compound (IIa) can be produced by reacting compound (31) with compound (32) and compound (33) in the presence of a base.

The amount of compound (33) to be used is generally 0.1 to 1.5 equivalents, preferably 0.8 to 1.1 equivalents, per 1 equivalent of compound (31).

The amount of compound (32) to be used is generally 0.1 to 1.5 equivalents, preferably 0.8 to 1.1 equivalents, per 1 equivalent of compound (31).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The amount of the base to be used is generally 2 to 1000 equivalents, preferably 2 to 20 equivalents, per 1 equivalent of compound (31).

The reaction temperature for this reaction is generally −78° C. to 200° C. The reaction time is generally 0.5 to 100 hr.

When and -Z^{a-2}-L^{a-2} and -Z^{a-2}-L^{a-2} of compound (31) are carboxy, the reaction with compound (32) and compound (33) is desirably carried out in the presence of a condensing agent in the same manner as in Reaction Scheme 1.

The amount of the condensing agent to be used is generally 2 to 1000 equivalents, preferably 2 to 20 equivalents, per 1 equivalent of compound (31).

The reaction may be carried out in the presence of a base in the same manner as in Reaction Scheme 1, if desired.

The amount of the base to be used is generally 2 to 1000 equivalents, preferably 2 to 20 equivalents, per 1 equivalent of compound (31).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (31). For example, compound (31) is reacted with an alkyl chlorocarbonate in the presence of a base in the same manner as in Reaction Scheme 1, and then the obtained mixed anhydride is reacted with compound (32) and compound (33).

The amount of the alkyl chlorocarbonate to be used is generally 2 to 1000 equivalents, preferably 2 to 20 equivalents, per 1 equivalent of compound (31).

The amount of the base to be used is generally 2 to 1000 equivalents, preferably 2 to 20 equivalents, per 1 equivalent of compound (31).

The reaction temperature for this reaction is generally −78° C. to 200° C. The reaction time is generally 0.5 to 100 hr.

Compound (31) may be commercially available, or can be synthesized according to a method known per se (e.g., the methods described in Comprehensive Organic Transformations, John Wiley and Sons (1999); Protective Groups in Organic Synthesis, John Wiley and Sons (1980)).

Compound (32) and compound (33) can be produced according to the method shown in Reaction Scheme 1, Reaction Scheme 10 or the below-mentioned Reaction Scheme 20.

Of compound (II), the following compound (IIb) can be produced, for example, according to the method shown in the following Reaction Scheme 12.

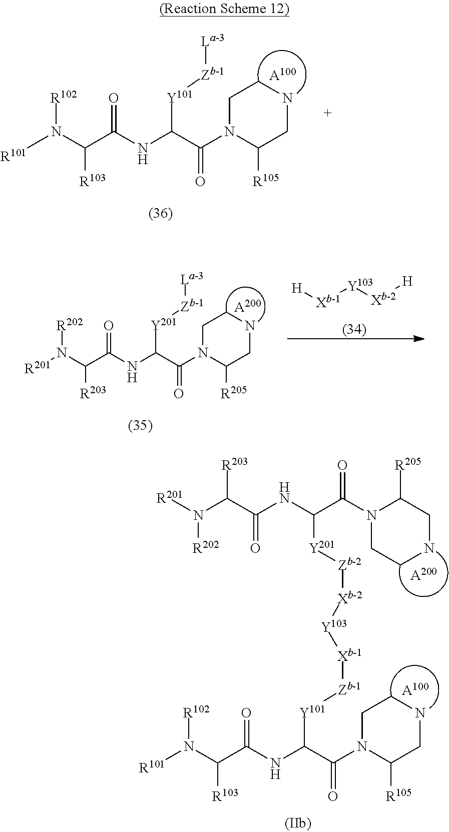

wherein X^{b-1} is a bond, —O—, —NR¹⁰⁶— or —S—, X^{b-2} is a bond, —O—, —NR²⁰⁶— or —S—, Z^{b-1} is a bond, —C(═O)—, —C(═O)—O—, —C(═O)—NR¹⁰⁶or —S(═O)₂—, Z^{b-2} is a bond, —C(═O)—, —C(═O)—O—, —C(═O)—NR²⁰⁶—, or —S(═O)₂—, L^{a-3} is a leaving group or carboxy, and the other symbols are each as defined above.

Examples of the leaving group for L^{a-3} include those exemplified as the above-mentioned L^{a-1}.

Compound (IIb) can be produced by reacting compound (34) with compound (35) and compound (36) in the presence of a base.

The amount of compound (35) to be used is generally 0.1 to 1.5 equivalents, preferably 0.8 to 1.1 equivalents, per 1 equivalent of compound (34).

The amount of compound (36) to be used is generally 0.1 to 1.5 equivalents, preferably 0.8 to 1.1 equivalents, per 1 equivalent of compound (34).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The amount of the base to be used is generally 2 to 1000 equivalents, preferably 2 to 20 equivalents, per 1 equivalent of compound (35).

The reaction temperature for this reaction is generally −78° C. to 200° C. The reaction time is generally 0.5 to 100 hr.

When -Z^{b-2}-L^{a-3} of compound (35) and -Z^{b-1}-L^{a-3} of compound (36) are carboxy, the reaction with compound (34) is desirably carried out in the presence of a condensing agent in the same manner as in Reaction Scheme 1.

The amount of the condensing agent to be used is generally 2 to 1000 equivalents, preferably 2 to 20 equivalents, per 1 equivalent of compound (34).

The reaction may be carried out in the presence of a base in the same manner as in Reaction Scheme 1, if desired.

The amount of the base to be used is generally 2 to 1000 equivalents, preferably 2 to 20 equivalents, per 1 equivalent of compound (34).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using mixed anhydrides of compound (35) and compound (36). For example, compound (35) and compound (36) are reacted with an alkyl chlorocarbonate in the presence of a base in the same manner as in Reaction Scheme 1, and then the obtained mixed anhydrides are reacted with compound (34).

The amount of the alkyl chlorocarbonate to be used is generally 2 to 1000 equivalents, preferably 2 to 20 equivalents, per 1 equivalent of compound (35) and compound (36).

The amount of the base to be used is generally 2 to 1000 equivalents, preferably 2 to 20 equivalents, per 1 equivalent of compound (35) and compound (36).

The reaction temperature for this reaction is generally −78° C. to 200° C. The reaction time is generally 0.5 to 100 hr.

Compound (34) may be commercially available, or can be synthesized according to a method known per se (e.g., the methods described in Comprehensive Organic Transformations, John Wiley and Sons (1999); Protective Groups in Organic Synthesis, John Wiley and Sons (1980)).

Compound (35) and compound (36) can be produced according to the method shown in Reaction Scheme 1, Reaction Scheme 10 or the below-mentioned Reaction Scheme 20.

Of compound (II), the following compound (IIc) can be produced, for example, according to the method shown in the following Reaction Scheme 13.

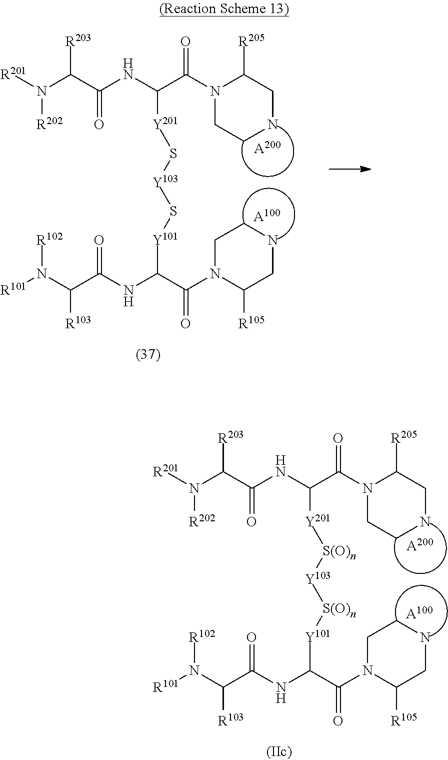

wherein n is an integer of 1 or 2, and the other symbols are each as defined above.

Compound (IIc) can be produced by reacting compound (37) with an oxidant.

Examples of the oxidant include m-chloroperbenzoic acid, hydrogen peroxide, peracetic acid, t-butyl hydroperoxide, potassium persulfate, potassium permanganate, sodium perborate, sodium periodate, sodium hypochlorite and halogen.

For the production of compound (IIc) wherein n=1, the oxidant is used in an amount of about 1 to 1.5 equivalents, per 1 equivalent of compound (37). For the production of compound (IIc) wherein n=2, the oxidant is used in an amount of about 2 to 3 equivalents, per 1 equivalent of compound (37).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (37) can be produced according to the method shown in Reaction Scheme 11 or Reaction Scheme 12.

Of compound (II), the following compound (IId) can be produced, for example, according to the method shown in the following Reaction Scheme 14.

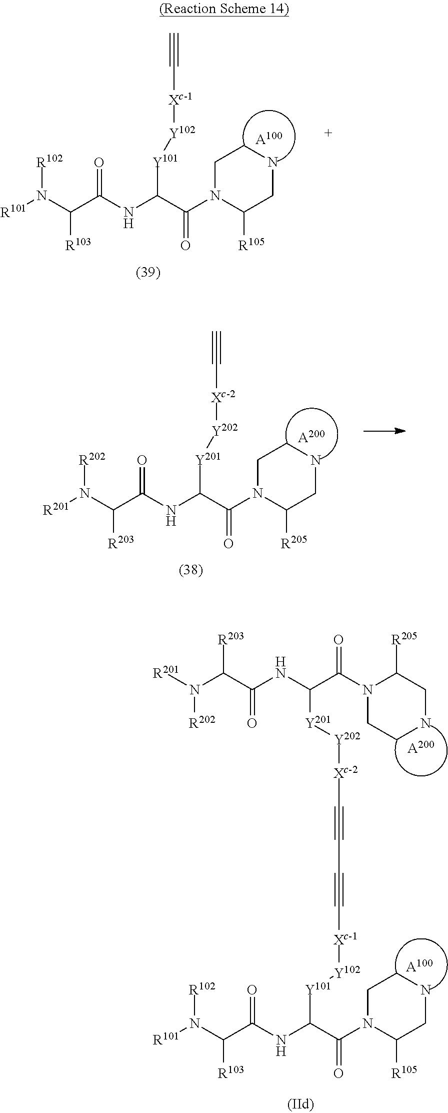

wherein X^{c-1} and X^{c-2} are each independently a bond, C₁₋₆ alkylene, C₂₋₆ alkenylene, C₂₋₆ alkynylene, C₃₋₆ cycloalkylene, C₆₋₁₀ arylene or a divalent heterocyclic group, and the other symbols are each as defined above.

Compound (IId) can be produced by subjecting compound (38) and compound (39) to a conventional coupling reaction with a copper catalyst (e.g., the methods described in U.S. Pat. No. 4,125,534; U.S. Pat. No. 6,350,875).

The amount of compound (39) to be used is generally 1 to 1000 equivalents, preferably 1 to 20 equivalents, per 1 equivalent of compound (38).

The amount of the copper catalyst to be used is generally 0.01 to 100 equivalents, preferably 0.1 to 10 equivalents, per 1 equivalent of compound (38).

This reaction is preferably carried out in a solvent inert to the reaction. The solvent is not particularly limited as long as the reaction proceeds, and examples thereof include alcohols, ethers, esters, halogenated hydrocarbons, hydrocarbons and nitriles. These solvents may be used in a mixture of two or more kinds thereof at an appropriate ratio.

The reaction temperature for this reaction is generally −78° C. to 200° C. The reaction time is generally 0.5 to 100 hr.

Compound (38) and compound (39) can be produced according to the method shown in Reaction Scheme 1, Reaction Scheme 10 or the below-mentioned Reaction Scheme 20.

Of compound (II), the following compound (IIe) can be produced, for example, according to the method shown in the following Reaction Scheme 15.

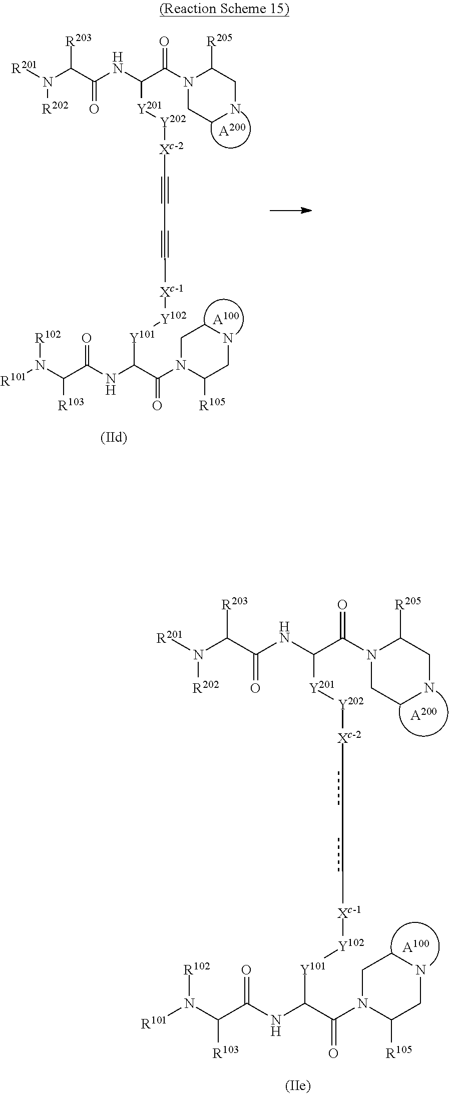

wherein each symbol is as defined above.

Compound (IIe) can be produced by subjecting compound (IId) to a reduction reaction known per se, for example, a hydrogenation reaction with a metal catalyst (e.g., the method described in Comprehensive Organic Transformations, John Wiley and Sons (1999)) to convert the alkyne of compound (IId) into the alkane or alkene.

Examples of the metal catalyst include a palladium catalyst, a platinum catalyst and a rhodium catalyst.

The amount of the metal catalyst to be used is generally 1 to 100 wt %, preferably 1 to 50 wt %, relative to compound (IId).

This reaction is preferably carried out in a solvent inert to the reaction. The solvent is not particularly limited as longus the reaction proceeds, and examples thereof include alcohols, ethers, esters, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (IId) can be produced according to the method shown in Reaction Scheme 14.

Of compound (II), the following compound (IIf) can be produced, for example, according to the method shown in the following Reaction Scheme 16.

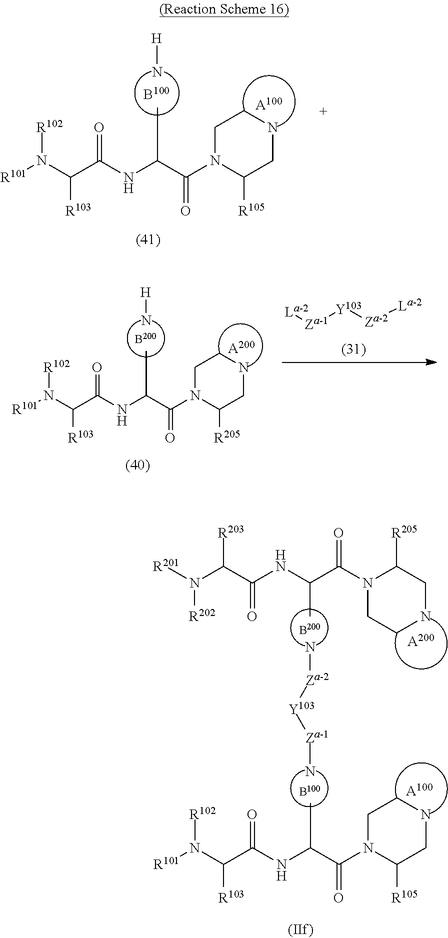

wherein B¹⁰⁰ is a divalent nitrogen-containing heterocycle optionally having substituent(s), B²⁰⁰ is a divalent nitrogen-containing heterocycle optionally having substituent(s), and the other symbols are each as defined above.

Examples of the “divalent nitrogen-containing heterocycle optionally having substituent(s)” for B¹⁰⁰ include a ring corresponding to the “divalent nitrogen-containing heterocyclic group optionally having substituent(s)” exemplified for —Y¹⁰¹—.

Examples of the “divalent nitrogen-containing heterocycle optionally having substituent(s)” for B²⁰⁰ include a ring corresponding to the “divalent nitrogen-containing heterocyclic group optionally having substituent(s)” exemplified for —Y¹⁰²—.

Compound (IIf) can be produced by reacting compound (31) with compound (40) and compound (41) in the presence of a base (e.g., triethylamine, N-methylmorpholine, N,N-dimethylaniline, sodium hydrogen carbonate, sodium carbonate, potassium carbonate).

The amount of compound (40) to be used is generally 0.1 to 1.5 equivalents, preferably 0.8 to 1.1 equivalents, per 1 equivalent of compound (31).

The amount of compound (41) to be used is generally 0.1 to 1.5 equivalents, preferably 0.8 to 1.1 equivalents, per 1 equivalent of compound (31).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The amount of the base to be used is generally 2 to 1000 equivalents, preferably 2 to 20 equivalents, per 1 equivalent of compound (31).

The reaction temperature for this reaction is generally −78° C. to 200° C. The reaction time is generally 0.5 to 100 hr.

When -Z^{a-1}-L^{a-2} and -Z^{a-2}-L^{a-2} of compound (31) are carboxy, the reaction with compound (40) and compound (41) is desirably carried out in the presence of a condensing agent in the same manner as in Reaction Scheme 1.

The amount of the condensing agent to be used is generally 2 to 1000 equivalents, preferably 2 to 20 equivalents, per 1 equivalent of compound (31).

The reaction may be carried out in the presence of a base in the same manner as in Reaction Scheme 1, if desired.

The amount of the base to be used is generally 2 to 1000 equivalents, preferably 2 to 20 equivalents, per 1 equivalent of compound (31).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (31). For example, compound (31) is reacted with an alkyl chlorocarbonate in the presence of a base in the same manner as in Reaction Scheme 1, and then the obtained mixed anhydride is reacted with compound (40) and compound (41).

The amount of the alkyl chlorocarbonate to be used is generally 2 to 1000 equivalents, preferably 2 to 20 equivalents, per 1 equivalent of compound (31).

The amount of the base to be used is generally 2 to 1000 equivalents, preferably 2 to 20 equivalents, per 1 equivalent of compound (31).

The reaction temperature for this reaction is generally −78° C. to 200° C. The reaction time is generally 0.5 to 100 hr.

Compound (31) may be commercially available, or can be synthesized according to a method known per se (e.g., the methods described in Comprehensive Organic Transformations, John Wiley and Sons (1999); Protective Groups in Organic Synthesis, John Wiley and Sons (1980)).

Compound (40) and compound (41) can be produced according to the method shown in Reaction Scheme 1, Reaction Scheme 10 or the below-mentioned Reaction Scheme 20.

Of compound (II), the following compound (IIg) can be produced, for example, according to the method shown in the following Reaction Scheme 17.

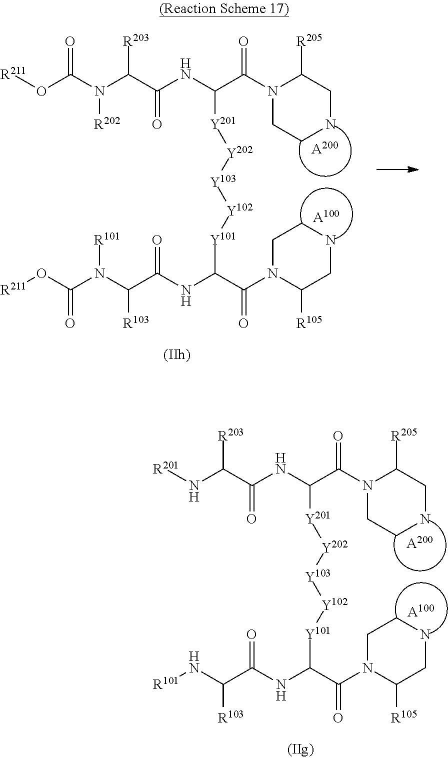

wherein each symbol is as defined above.

Compound (IIg) can be produced by converting the carbamate of compound (IIh) into the amine.

The conversion from the carbamate into the amine can be carried out according to a method known per se (e.g., the method described in Protective Groups in Organic Synthesis, John Wiley and Sons (1980)).

Specifically, a method using an acid, a base and the like, a reduction method and the like can be employed.

Compound (IIh) can be produced according to the method shown in Reaction Scheme 11 to 16.

Of compound (29), the following compound (29a) can be produced, for example, according to the method shown in the following Reaction Scheme 18.

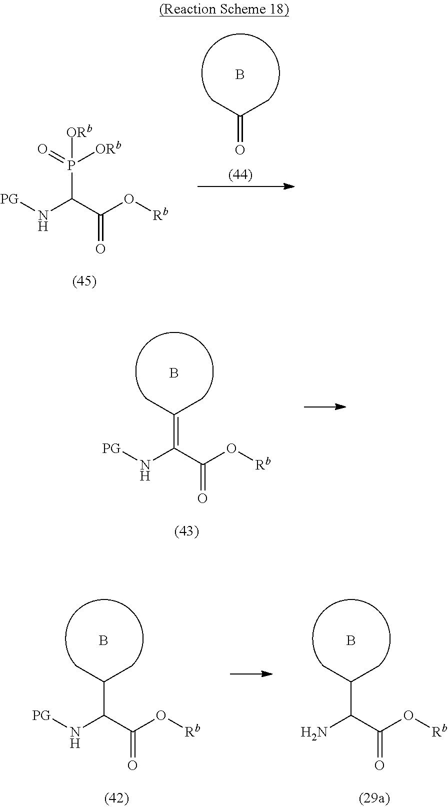

wherein ring B is a cyclic group optionally having substituent(s), and the other symbols are each as defined above.

Examples of the “cyclic group optionally having substituent(s)” for ring B include those exemplified for the above-mentioned R⁴.

Compound (43) can be produced by reacting compound (45) with compound (44) in the presence of a base (e.g., tetramethylguanidine, triethylamine, N-methylmorpholine, N,N-dimethylaniline, sodium hydrogen carbonate, sodium carbonate, potassium carbonate) according to a method known per se (e.g., the method described in Tetrahedron: Asymmetry, 2001, 12, 2421-2425 and the like).

The amount of compound (45) to be used is generally 1 to 1000 equivalents, preferably 1 to 20 equivalents, per 1 equivalent of compound (44).

This reaction is preferably carried out in a solvent inert to the reaction. The solvent is not particularly limited as long as the reaction proceeds, and examples thereof include ethers, esters, hydrocarbons, amides, nitriles, sulfoxides, sulfolane and hexamethylphosphoramide. These solvents may be used in a mixture of two or more kinds thereof at an appropriate ratio.

The amount of the base to be used is generally 1 to 1000 equivalents, preferably 1 to 20 equivalents, per 1 equivalent of compound (45).

The reaction temperature for this reaction is generally −78° C. to 200° C. The reaction time is generally 0.5 to 100 hr.

Compound (42) can be produced by converting the alkene of compound (43) into the alkane in the same manner as in Reaction Scheme 15.

Specifically, compound (42) can be produced by subjecting compound (43) to an asymmetric hydrogenation reaction known per se with a metal catalyst and an asymmetric ligand (e.g., the methods described in J. Am. Chem. Soc. 1995, 117, 9375-9375; Tetrahedron: Asymmetry, 2001, 12, 2421-2425; WO 2008/79735 and the like) to stereoselectively convert the alkene of compound (43) into the alkane.

Examples of the metal catalyst include a rhodium catalyst and a ruthenium catalyst.

Examples of the asymmetric ligand include (+)-1,2-bis[(2S,5S)-2,5-dimethylphospholano]benzene, (−)-1,2-bis[(2S,5S)-2,5-dimethylphospholano]ethane, (−)-1,2-bis[(2R,5R)-2,5-dimethylphospholano]benzene, bis[(2R,5R)-2,5-dimethylphospholano]ethane and the like.

The amount of the metal catalyst to be used is generally 1 to 100 wt %, preferably 1 to 50 wt %, relative to compound (43).

The amount of the asymmetric ligand to be used is generally 1 to 100 wt %, preferably 1 to 50 wt %, relative to compound (43).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (29a) can be produced by removing the PG of compound (42) in the same manner as in the production method of compound (2a) from compound (4a) in Reaction Scheme 2.

Compound (44) may be commercially available, or can be synthesized according to a method known per se.

Compound (45) may be commercially available, or can be synthesized according to a method known per se (e.g., the method described in J. Org. Chem., 2002, 67, 620-624).

Compound (5) can be produced, for example, according to the method shown in the following Reaction Scheme 19.

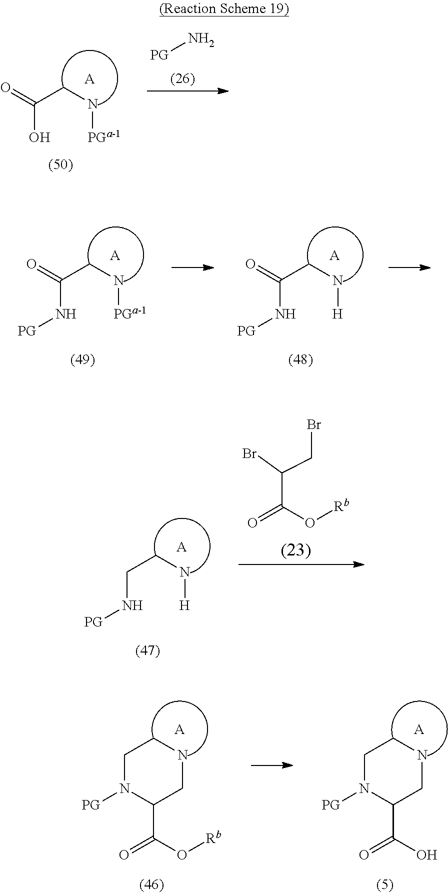

wherein PG^{a-1} is an amino-protecting group, and the other symbols are each as defined above.

Examples of the amino-protecting group for PG^{a-1} include those exemplified as the above-mentioned PG.

Compound (49) can be produced by reacting compound (50) with compound (26).

The amount of compound (50) to be used is generally 1 to 1000 equivalents, preferably 1 to 20 equivalents, per 1 equivalent of compound (26).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (26).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (26).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (50). For example, compound (50) is reacted with an alkyl chlorocarbonate in the presence of a base in the same manner as in Reaction Scheme 1, and then the obtained mixed anhydride is reacted with compound (26).

The amount of the alkyl chlorocarbonate to be used is generally 1 to 1000 equivalents, preferably 1 to 20 equivalents, per 1 equivalent of compound (26).

The amount of the base to be used is generally 1 to 1000 equivalents, preferably 1 to 20 equivalents, per 1 equivalent of compound (26).

The reaction temperature for this reaction is generally −78° C. to 200° C. The reaction time is generally 0.5 to 100 hr.

Compound (48) can be produced by removing the PG^{a-1} of compound (49) in the same manner as in the production method of compound (2a) from compound (4a) in Reaction Scheme 2.

Compound (47) can be produced by subjecting compound (48) to a reduction reaction in the same manner as in the production method of compound (4b) from compound (6) in Reaction Scheme 3 to convert the carbonyl of compound (48) into methylene.

The amount of the reducing agent to be used is generally 1 to 1000 equivalents, preferably 1 to 20 equivalents, per 1 equivalent of compound (48).

The reaction temperature for this reaction is generally −78° C. to 200° C. The reaction time is generally 0.5 to 100 hr.

Compound (46) can be produced by reacting compound (47) with compound (23) in the presence of a base (e.g., triethylamine, N-methylmorpholine, N,N-dimethylaniline, sodium hydrogen carbonate, sodium carbonate, potassium carbonate).

The amount of compound (23) to be used is generally 1 to 1000 equivalents, preferably 1 to 20 equivalents, per 1 equivalent of compound (47).

This reaction is preferably carried out in a solvent inert to the reaction. The solvent is not particularly limited as long as the reaction proceeds, and examples thereof include ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The amount of the base to be used is generally 1 to 1000 equivalents, preferably 1 to 20 equivalents, per 1 equivalent of compound (47).

The reaction temperature for this reaction is generally −78° C. to 200° C. The reaction time is generally 0.5 to 100 hr.

Compound (5) can be produced by converting compound (46) into the carboxylic acid in the same manner as in the production method of compound (14) from compound (15) in Reaction Scheme 6.

Specifically, compound (5) can be produced by reacting compound (46) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (46).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

In this reaction, the PG of compound (46) and the PG of compound (5) are the same or different. When the PG of compound (46) and the PG of compound (5) are different, the PG of compound (46) is removed according to a method known per se (e.g., the method described in Protective Groups in Organic Synthesis, John Wiley and Sons (1980)), and then the desired PG is introduced into compound (46) according to a method known per se (e.g., the method described in Protective Groups in Organic Synthesis, John Wiley and Sons (1980)).

Compound (50) may be commercially available, or can be synthesized according to a method known per se (e.g., introduction and removal of protecting group according to the method described in WO 2006/038119; the method described in Protective Groups in Organic Synthesis, John Wiley and Sons (1980)).

Compound (Ia) can also be produced, for example, according to the method shown in the following Reaction Scheme 20 or an analogous method thereto.

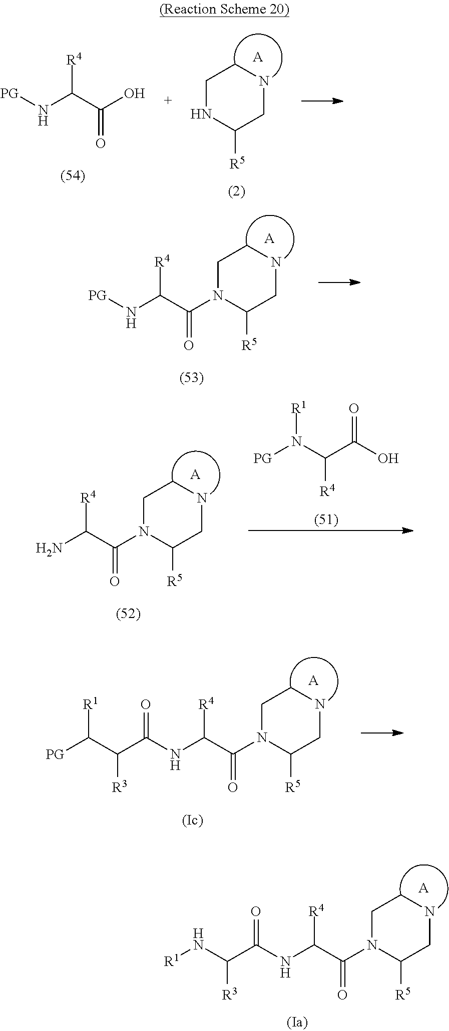

wherein each symbol is as defined above.

Compound (53) can be produced by reacting compound (2) with compound, (54).

The amount of compound (54) to be used is generally 1 to 1000 equivalents, preferably 1 to 20 equivalents, per 1 equivalent of compound (2).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (2).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (2).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (54). For example, compound (54) is reacted with an alkyl chlorocarbonate in the presence of a base in the same manner as in Reaction Scheme 1, and then the obtained mixed anhydride is reacted with compound (2).

The amount of the alkyl chlorocarbonate to be used is generally 1 to 1000 equivalents, preferably 1 to 20 equivalents, per 1 equivalent of compound (2).

The amount of the base to be used is generally 1 to 1000 equivalents, preferably 1 to 20 equivalents, per 1 equivalent of compound (2).

The reaction temperature for this reaction is generally −78° C. to 200° C. The reaction time is generally 0.5 to 100 hr.

Compound (54) may be commercially available, or can be synthesized according to a method known per se (e.g., the methods described in J. Heterocyclic Chem., 1991, 28, 1715-1720; WO 2008/85302; J. Am. Chem. Soc. 1995, 117, 9375-9376; J. Med. Chem., 1993, 36, 2300-2310; Tetrahedron: Asymmetry, 2001, 12, 2421-2425; WO 2009/005677; WO 2006/069063; US 2003/0216325 and the like; the method described in the below-mentioned Reaction Scheme 21; the method for introduction and removal of protecting groups described in Protective Groups in Organic Synthesis, John Wiley and Sons (1980)).

Compound (52) can be produced by removing the PG of compound (53) in the same manner as in the production method of compound (2a) from compound (4a) in Reaction Scheme 2.

Compound (Ic) can be produced by reacting compound (52) with compound (51).

The amount of compound (51) to be used is generally 1 to 1000 equivalents, preferably 1 to 20 equivalents, per 1 equivalent of compound (52).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (52).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (52).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (51). For example, compound (51) is reacted with an alkyl chlorocarbonate in the presence of a base in the same manner as in Reaction Scheme 1, and then the obtained mixed anhydride is reacted with compound (52).

The amount of the alkyl chlorocarbonate to be used is generally 1 to 1000 equivalents, preferably 1 to 20 equivalents, per 1 equivalent of compound (52).

The amount of the base to be used is generally 1 to 1000 equivalents, preferably 1 to 20 equivalents, per 1 equivalent of compound (52).

The reaction temperature for this reaction is generally −78° C. to 200° C. The reaction time is generally 0.5 to 100 hr.

Compound (51) may be commercially available, or can be synthesized according to a method known per se (e.g., the methods described in J. Heterocyclic Chem., 1991, 28, 1715-1720; WO 2008/85302; J. Am. Chem. Soc. 1995, 117, 9375-9376; J. Med. Chem., 1993, 36, 2300-2310; Tetrahedron: Asymmetry, 2001, 12, 2421-2425; U.S. Pat. No. 6,436,904; US 2009/123423; Eur. J. Org. Chem., 2003, 4757-4764; J. Org. Chem., 2003, 68, 7033-7040; WO 2009/005677; WO 2006/069063; US 2003/0216325 and the like; the method for introduction and removal of protecting groups described in Protective Groups in Organic Synthesis, John Wiley and Sons (1980)).

Compound (Ia) can be produced by removing the PG of compound (Ic) in the same manner as in the production method of compound (2a) from compound (4a) in Reaction Scheme 2.

Of compound (54), the following compound (54a) can be produced, for example, according to the method shown in the following Reaction Scheme 21.

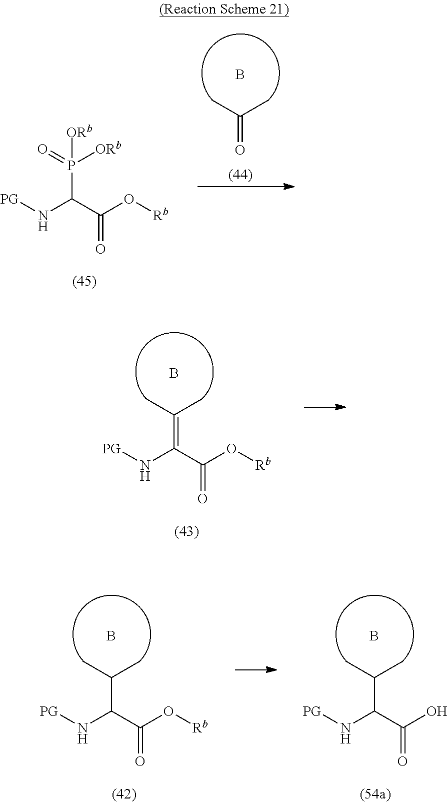

wherein each symbol is as defined above.

Compound (43) can be produced by reacting compound (45) with compound (44) in the presence of a base (e.g., tetramethylguanidine, triethylamine, N-methylmorpholine, N,N-dimethylaniline, sodium hydrogen carbonate, sodium carbonate, potassium carbonate) according to a method known per se (e.g., the method described in Tetrahedron: Asymmetry, 2001, 12, 2421-2425 and the like).

The amount of compound (45) to be used is generally 1 to 1000 equivalents, preferably 1 to 20 equivalents, per 1 equivalent of compound (44).

This reaction is preferably carried out in a solvent inert to the reaction. The solvent is not particularly limited as long as the reaction proceeds, and examples thereof include ethers, esters, hydrocarbons, amides, nitriles, sulfoxides, sulfolane and hexamethylphosphoramide. These solvents may be used in a mixture of two or more kinds thereof at an appropriate ratio.

The amount of the base to be used is generally 1 to 1000 equivalents, preferably 1 to 20 equivalents, per 1 equivalent of compound (45).

The reaction temperature for this reaction is generally −78° C. to 200° C. The reaction time is generally 0.5 to 100 hr.

Compound (42) can be produced by converting the alkene of compound (43) into the alkane in the same manner as in Reaction Scheme 15.

Specifically, compound (42) can be produced by subjecting compound (43) to an asymmetric hydrogenation reaction known per se with a metal catalyst and an asymmetric ligand (e.g., the methods described in J. Am. Chem. Soc. 1995, 117, 9375-9376; Tetrahedron: Asymmetry, 2001, 12, 2421-2425; W2008/79735 and the like) to stereoselectively convert the alkene of compound (43) into the alkane.

Examples of the metal catalyst include a rhodium catalyst and a ruthenium catalyst.

Examples of the asymmetric ligand include (+)-1,2-bis[(2S,5S)-2,5-dimethylphospholano]benzene, (−)-1,2-bis[(2S,5S)-2,5-dimethylphospholano]ethane, (−)-1,2-bis[(2R,5R)-2,5-dimethylphospholano]benzene and (+)-1,2-bis[(2R,5R)-2,5-dimethylphospholano]ethane.

The amount of the metal catalyst to be used is generally 1 to 100 wt %, preferably 1 to 50 wt %, relative to compound (43).

The amount of the asymmetric ligand to be used is generally 1 to 100 wt %, preferably 1 to 50 wt %, relative to compound (43).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (54a) can be produced by converting compound (42) into the carboxylic acid in the same manner as in the production method of compound (14) from compound (15) in Reaction Scheme 6.

Specifically, compound (54a) can be produced by reacting compound (42) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (42).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Of compound (I), the following compound (Id) can be produced, for example, according to the method shown in the following Reaction Scheme 22.

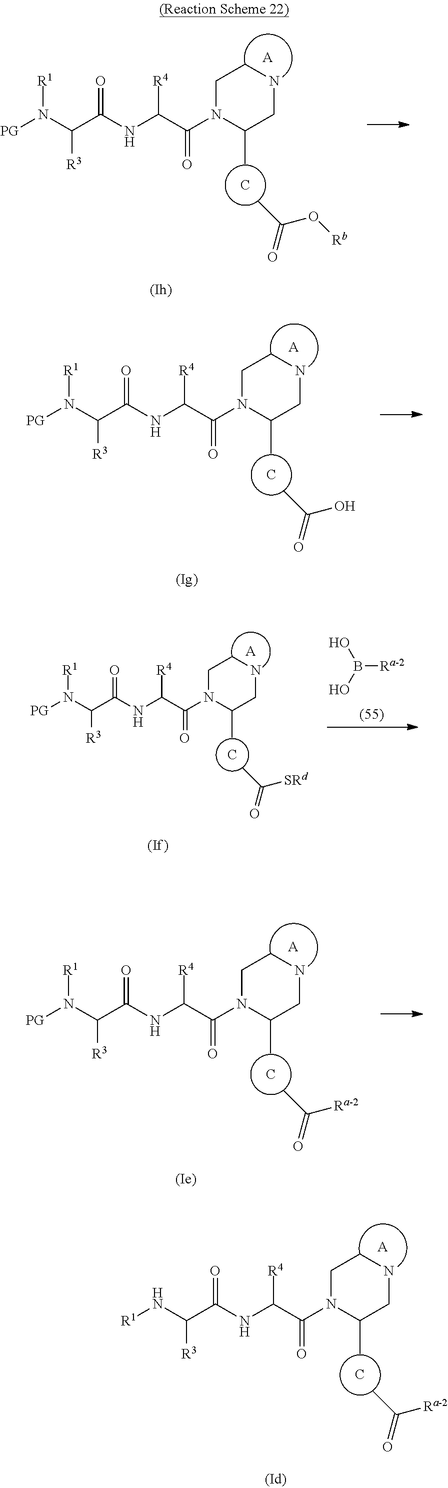

wherein ring C is a cyclic group optionally having substituent(s), R^{d} is C₁₋₁₀ alkyl or C₆₋₁₀ aryl, each optionally having substituent(s), and the other symbols are each as defined above.

Compound (Ig) can be produced by converting compound (Ih) into the carboxylic acid in the same manner as in the production method of compound (14) from compound (15) in Reaction Scheme 6.

Specifically, compound (Ig) can be produced by reacting compound (Ih) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (Ih).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (If) can be produced by reacting compound (Ig) with a thiol represented by R^{d}—SH.

The amount of the thiol to be used is generally 1 to 1000 equivalents, preferably 1 to 20 equivalents, per 1 equivalent of compound (Ig).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (Ig).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (Ig).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (Ie) can be produced by reacting compound (If) with boronic acid (55) according to a method known per se (e.g., the method described in J. Am. Chem. Soc., 2000, 122, 11260-11261).

The amount of the boronic acid (55) to be used is generally 1 to 1000 equivalents, preferably 1 to 20 equivalents, per 1 equivalent of compound (If).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Boronic acid (55) may be commercially available, or can be synthesized according to a method known per se (e.g., the method described in Tetrahedron, 1963, 19, 821-826).

Compound (Id) can be produced by removing the PG of compound (Ie) in the same manner as in the production method of compound (2a) from compound (4a) in Reaction Scheme 2.

Compound (Ih) can be produced from compound (2d) synthesized according to the method shown in the above-mentioned Reaction Scheme 5, compound (2h) synthesized according to the method shown in the below-mentioned Reaction Scheme 25, or compound (2i) synthesized according to the method shown in the below-mentioned Reaction Scheme 26, in the same manner as in the production method of compound (I) from compound (2) in the above-mentioned Reaction Scheme 1.

Of compound (2), the following compound (2g) can be produced, for example, according to the method shown in the following Reaction Scheme 23.

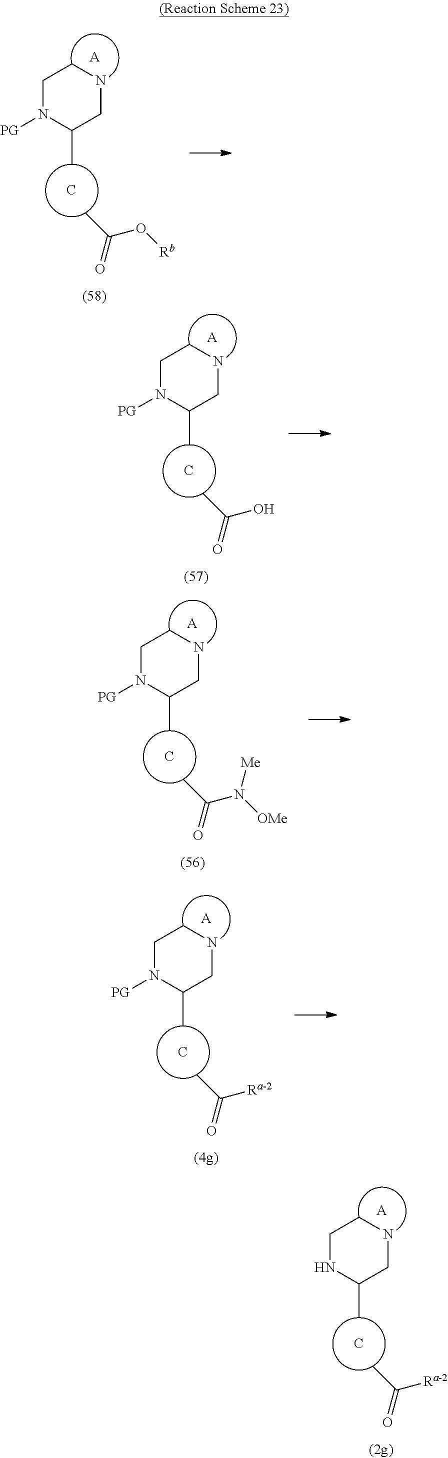

wherein each symbol is as defined above.

Compound (57) can be produced by converting compound (58) into the carboxylic acid in the same manner as in the production method of compound (14) from compound (15) in Reaction Scheme 6.

Specifically, compound (57) can be produced by reacting with compound (58) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (58).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (56) (i.e., Weinreb amide) can be produced by reacting compound (57) with N,O-dimethylhydroxylamine (or a salt thereof) in the same manner as in the production method of compound (13) from compound (14) in Reaction Scheme 6. The amount of the N,O-dimethylhydroxylamine (or a salt thereof) to be used is generally 1 to 1000 equivalents, preferably 1 to 20 equivalents, per 1 equivalent of compound (57).

The reaction is desirably carried out in the presence of a condensing agent in the same manner as in Reaction Scheme 1. The amount of the condensing agent to be used is generally 1 to 1000 equivalents, preferably 1 to 20 equivalents, per 1 equivalent of compound (57).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (57).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (57). For example, compound (57) is reacted with an alkyl chlorocarbonate in the presence of a base in the same manner as in Reaction Scheme 1, and then the obtained mixed anhydride is reacted with N,O-dimethylhydroxylamine (or a salt thereof).

The amount of the alkyl chlorocarbonate to be used is generally 1 to 1000 equivalents, preferably 1 to 20 equivalents, per 1 equivalent of compound (57).

The amount of the base to be used is generally 1 to 1000 equivalents, preferably 1 to 20 equivalents, per 1 equivalent of compound (57).

The reaction temperature for this reaction is generally −78° C. to 200° C. The reaction time is generally 0.5 to 100 hr. [0290]

Compound (4g) can be produced by reacting compound (56) with a reagent corresponding to R^{a-2} in the same manner as in the production method of compound (4e) from compound (13) in Reaction Scheme 6.

Examples of the reagent include a magnesium reagent corresponding to R^{a-2}, and a lithium reagent corresponding to R^{a-2}.

The amount of the reagent corresponding to R^{a-2} to be used is generally 1 to 1000 equivalents, preferably 1 to 20 equivalents, per 1 equivalent of compound (56).

This reaction is preferably carried out in a solvent inert to the reaction. The solvent is not particularly limited as long as the reaction proceeds, and examples thereof include ethers, hydrocarbons and the like. These solvents may be used in a mixture of two or more kinds thereof at an appropriate ratio.

The reaction temperature for this reaction is generally −78° C. to 200° C. The reaction time is generally 0.5 to 100 hr.

Compound (2g) can be produced by removing the PG of compound (4g) in the same manner as in the production method of compound (2a) from compound (4a) in Reaction Scheme 2.

Compound (58) can be produced using compound (10) wherein R^{a-1} is COOR^{b} in Reaction Scheme 5, compound (65) wherein R^{a-1} is COOR^{b} in the below-mentioned Reaction Scheme 25, compound (66) wherein R^{a-1} is COOR^{b} in the below-mentioned Reaction Scheme 26.

Of compound (54), the following compound (54b) can be produced, for example, according to the method shown in the following Reaction Scheme 24.

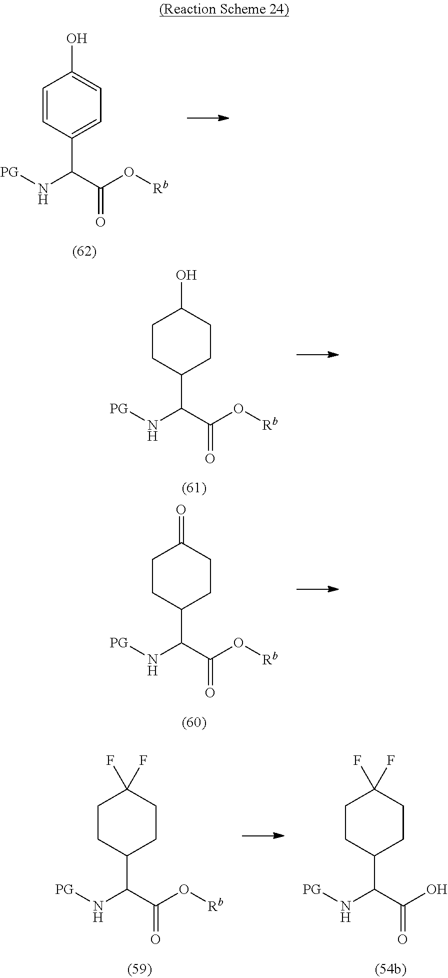

wherein each symbol is as defined above.

Compound (61) can be produced by subjecting compound (62) to a reduction reaction known per se, for example, a hydrogenation reaction with a metal catalyst (e.g., the methods described in US 2005/234065; WO 2004/110436; EP 1695969; J. Am. Chem. Soc., 1958, 80, 2698-2700; Comprehensive Organic Transformations, John Wiley and Sons (1999)) to convert the phenyl group of compound (62) into cyclohexyl group.

Examples of the metal catalyst include a palladium catalyst, a platinum catalyst, rhodium catalyst and nickel catalyst.

The amount of the metal catalyst to be used is generally 1 to 100 wt %, preferably 1 to 50 wt %, relative to compound (62).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr.

Compound (60) can be produced by subjecting compound (61) to an oxidation reaction known per se with an oxidant (e.g., the method described in Comprehensive Organic Transformations, John Wiley and Sons (1999)) to convert the hydroxy group of compound (61) into an oxo group.

Examples of the oxidant include Jones reagent, pyridinium chlorochromate, pyridinium dichromate, dimethyl sulfoxide/oxalyl chloride (Swern method), dimethyl sulfoxide/trifluoroacetic anhydride (Swern method), iodobenzene diacetate, Dess-Martin periodinane and TPAP (tetrapropylammonium perruthenate).

The amount of the oxidant to be used is generally 1 to 1000 equivalents, preferably 1 to 20 equivalents, per 1 equivalent of compound (61).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally, −78° C. to 200° C. The reaction time is generally 0.5 to 100 hr.

Compound (59) can be produced by converting the carbonyl of the oxo-substituted cyclohexyl moiety of compound (60) into difluoromethylene.

The conversion from the carbonyl into the difluoromethylene can be carried out according to a method known per se (e.g., the methods described in J. Org. Chem. 1999, 64, 7048-7054; US 2003/0216325 and the like).

Specifically, the conversion can be carried out using a fluorinating agent and the like.

Examples of the fluorinating agent include (dimethylamino)sulfur trifluoride, (diethylamino)sulfur trifluoride, bis(2-methoxyethyl)aminosulfur trifluoride.

The amount of the fluorinating agent to be used is generally 1 to 1000 equivalents, preferably 1 to 20 equivalents, per 1 equivalent of compound (60).

This reaction is preferably carried out in a solvent inert to the reaction. The solvent is not particularly limited as long as the reaction proceeds, and examples thereof include ethers, halogenated hydrocarbons, hydrocarbons and nitriles. These solvents may be used in a mixture of two or more kinds thereof at an appropriate ratio.

The reaction temperature for this reaction is generally −78° C. to 200° C. The reaction time is generally 0.5 to 100 hr.

Compound (54b) can be produced by converting compound (59) into the carboxylic acid in the same manner as in the production method of compound (14) from compound (15) in Reaction Scheme 6.

Specifically, compound (54b) can be produced by reacting compound (59) with an alkali metal hydroxide or an alkaline earth metal hydroxide.

The amount of the alkali metal hydroxide or alkaline earth metal hydroxide to be used is generally 1 to 1000 equivalents, preferably 1 to 20 equivalents, per 1 equivalent of compound (59).

This reaction is preferably carried out in a solvent inert to the reaction. The solvent is not particularly limited as long as the reaction proceeds, and examples thereof include alcohols, ethers, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr. [0299]

Compound (62) can be produced in the same manner as in the production method of compound (42) in Reaction Scheme 18 and Reaction Scheme 21.

Of compound (2), the following compound (2h) can be produced, for example, according to the method shown in the following Reaction Scheme 25.

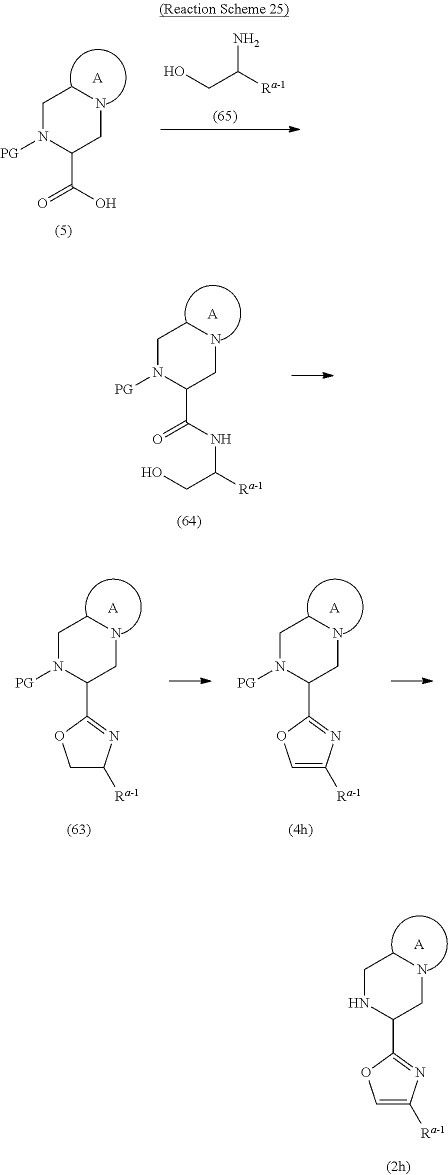

wherein each symbol is as defined above.

Compound (64) can be produced by reacting compound (5) with compound (65).

The amount of compound (65) to be used is generally 1 to 1000 equivalents, preferably 1 to 20 equivalents, per 1 equivalent of compound (5).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (5).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (5).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with compound (65).

The amount of the alkyl chlorocarbonate to be used is generally 1 to 1000 equivalents, preferably 1 to 20 equivalents, per 1 equivalent of compound (5).

The amount of the base to be used is generally 1 to 1000 equivalents, preferably 1 to 20 equivalents, per 1 equivalent of compound (5).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (63) can be produced by subjecting compound (64) to a intramolecular condensation using Burgess reagent according to a method known per se (e.g., the method described in J. Am. Chem. Soc., 2004, 126, 12888-12896 and the like).

The amount of the Burgess reagent to be used is generally 1 to 1000 equivalents, preferably 1 to 20 equivalents, per 1 equivalent of compound (64).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (65) may be commercially available.

Compound (4h) can be produced by subjecting compound (63) to a dehydrogenative oxidation reaction known per se with an oxidant (e.g., the methods described in J. Am. Chem. Soc., 2004, 126, 12888-12896; Comprehensive Organic Transformations, John Wiley and Sons (1999) and the like).

Examples of the oxidant include bromotrichloromethane/diazabicycloundecene.

The amount of the oxidant to be used is generally 1 to 1000 equivalents, preferably 1 to 20 equivalents, per 1 equivalent of compound (63).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane, hexamethylphosphoramide and water. These solvents may be used in a mixture of two or more kinds thereof at an appropriate ratio.

The reaction temperature for this reaction is generally −78° C. to 200° C. The reaction time is generally 0.5 to 100 hr. [0307]

Compound (2h) can be produced by removing the PG of compound (4h) in the same manner as in the production method of compound (2a) from compound (4a) in Reaction Scheme 2.

Of compound (2), the following compound (21) can be produced, for example, according to the method shown in the following Reaction Scheme 26.

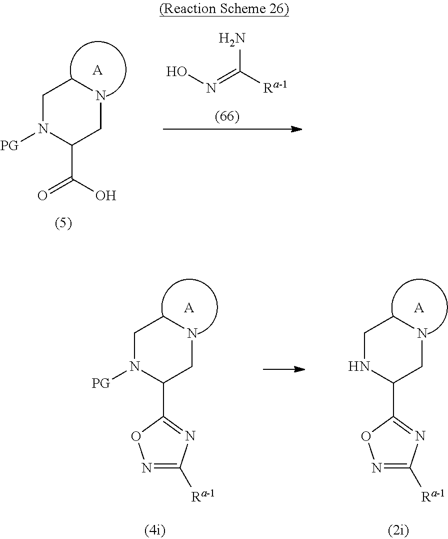

wherein each symbol is as defined above.

Compound (41) can be produced by reacting compound (5) with compound (66) according to a method known per se (e.g., the methods described in Tetrahedron Lett., 1996, 37, 6627-6630; Bioorg. Med. Chem. Lett., 1999, 9, 209-212; Tetrahedron Lett., 2001, 42, 1495-1498; J. Org. Chem., 2004, 69, 1470-1474 and the like).

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (5).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (5).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (66) may be commercially available.

Compound (21) can be produced by removing the PG of compound (41) in the same manner as in the production method of compound (2a) from compound (4a) in Reaction Scheme 2.

Of compound (2), the following compound (2j) can be produced, for example, according to the method shown in the following Reaction Scheme 27.

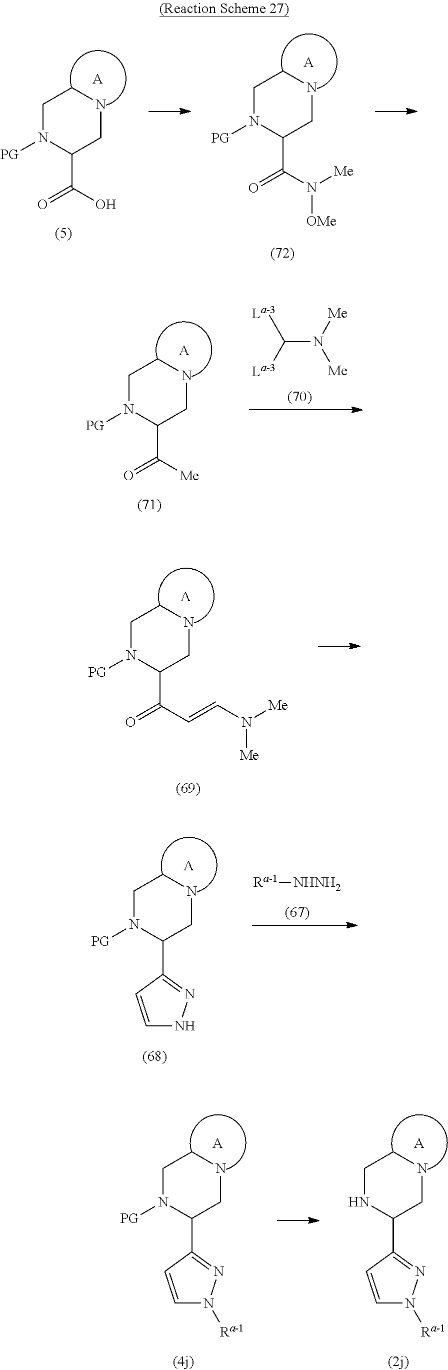

wherein L^{a-3} is a leaving group, and the other symbols are as defined above.

Examples of the leaving group for L^{a-3} include a group represented by the formula —OR^{aa} and a group represented by the formula —N(R^{aa})₂ wherein R^{aa} is C₁₋₄ alkyl (e.g., methyl, ethyl, propyl), benzyl or C₆₋₁₀ aryl (e.g., phenyl, tolyl).

Compound (72) (i.e., Weinreb amide) can be produced by reacting compound (5) with N,O-dimethylhydroxylamine (or a salt thereof).

The amount of the N,O-dimethylhydroxylamine (or a salt thereof) to be used is generally 1 to 1000 equivalents, preferably 1 to 20 equivalents, per 1 equivalent of compound (5).

N,O-Dimethylhydroxylamine (or a salt thereof) may be commercially available, or can be produced according to a method known per se.

The reaction is desirably carried out in the presence of a condensing agent in the same manner as in Reaction Scheme 1.

The amount of the condensing agent to be used is generally 1 to 1000 equivalents, preferably 1 to 20 equivalents, per 1 equivalent of compound (5).

The reaction may be carried out in the presence of a base in the same manner as in Reaction Scheme 1, if desired.

The amount of the base to be used is generally 1 to 1000 equivalents, preferably 1 to 20 equivalents, per 1 equivalent of compound (5).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Alternatively, this reaction may be carried out using a mixed anhydride of compound (5). For example, compound (5) is reacted with an alkyl chlorocarbonate in the presence of a base in the same manner as in Reaction Scheme 1, and then the obtained mixed anhydride is reacted with N,O-dimethylhydroxylamine (or a salt thereof).

The amount of the alkyl chlorocarbonate to be used is generally 1 to 1000 equivalents, preferably 1 to 20 equivalents, per 1 equivalent of compound (5).

The amount of the base to be used is generally 1 to 1000 equivalents, preferably 1 to 20 equivalents, per 1 equivalent of compound (5).

The reaction temperature for this reaction is generally −78° C. to 200° C. The reaction time is generally 0.5 to 100 hr.

Compound (71) can be produced by reacting compound (72) with a methylating agent according to a method known per se (e.g., the method described in Tetrahedron Letters, 1981, 22, 3815-3818).

Examples of the methylating agent include a methylmagnesium reagent, a methyllithium reagent and the like.

The methylmagnesium reagent may be commercially available as a Grignard reagent, or can be synthesized according to a method known per se (e.g., the method described in Heterocycles, 1987, 26, 3141-3151).

The methyllithium reagent may be commercially available, or can be synthesized according to a method known per se (e.g., the method described in J. Org. Chem., 1991, 56, 3750-3752).

The amount of the methylating agent to be used is generally 1 to 1000 equivalents, preferably 1 to 20 equivalents, per 1 equivalent of compound (72).

This reaction is preferably carried out in a solvent inert to the reaction. The solvent is not particularly limited as long as the reaction proceeds, and examples thereof include ethers, hydrocarbons and the like. These solvents may be used in a mixture of two or more kinds thereof at an appropriate ratio.

The reaction temperature for this reaction is generally −78° C. to 200° C. The reaction time is generally 0.5 to 100 hr. [0318]

Compound (69) can be produced by reacting compound (71) with compound (70) according to a method known per se (e.g., the methods described in US 2003/225157; WO 2003/101993; U.S. Pat. No. 5,292,766 and the like).

Examples of compound (70) include known compounds such as N,N-dimethylformamide dimethylacetal, tert-butoxy bis(dimethylamino)methane, tris(dimethylamino)methane and the like.

The amount of compound (70) to be used is generally 1 to 1000 equivalents, preferably 1 to 20 equivalents, per 1 equivalent of compound (71).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (68) can be produced by reacting compound (69) with a hydrazine according to a method known per se (e.g., the methods described in U.S. Pat. No. 6,511,974; US 2009/202480; U.S. Pat. No. 5,932,745 and the like).

Examples of the hydrazine include hydrazine non-hydrate, hydrazine monohydrate, hydrazine monohydrochloride and the like.

The amount of the hydrazine to be used is generally 1 to 1000 equivalents, preferably 1 to 20 equivalents, per 1 equivalent of compound (69).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (4j) can be produced by reacting compound (68) with compound (67) in the presence of a base (e.g., triethylamine, N-methylmorpholine, N,N-dimethylaniline, sodium hydrogen carbonate, sodium carbonate, potassium carbonate) according to a method known per se (e.g., the methods described in WO 2005/49578; WO 2007/18314; WO 2008/74820 and the like).

The amount of compound (67) to be used is generally 1 to 1000 equivalents, preferably 1 to 20 equivalents, per 1 equivalent of compound (68).

The amount of the base to be used is generally 1 to 1000 equivalents, preferably 1 to 20 equivalents, per 1 equivalent of compound (68).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (67) may be commercially available.

Compound (2j) can be produced by removing the PG of compound (4j) in the same manner as in the production method of compound (2a) from compound (4a) in Reaction Scheme 2.

Of compound (2), the following compound (2k) can be produced, for example, according to the method shown in the following Reaction Scheme 28.

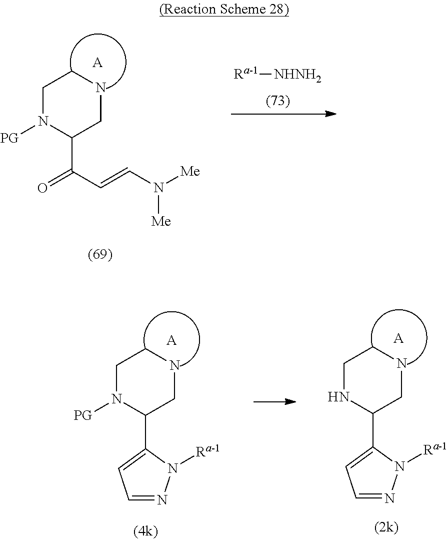

wherein each symbol is as defined above.

Compound (4k) can be produced by reacting compound (69) with compound (73) according to a method known per se (e.g., the method described in U.S. Pat. No. 6,414,011; WO 2007/94513 and the like).

The amount of compound (73) to be used is generally 1 to 1000 equivalents, preferably 1 to 20 equivalents, per 1 equivalent of compound (69).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (73) may be commercially available.

Compound (2k) can be produced by removing the PG of compound (4k) in the same manner as in the production method of compound (2a) from compound (4a) in Reaction Scheme 2.

Compound (46) can also be produced according to the method shown in the following Reaction Scheme 29.

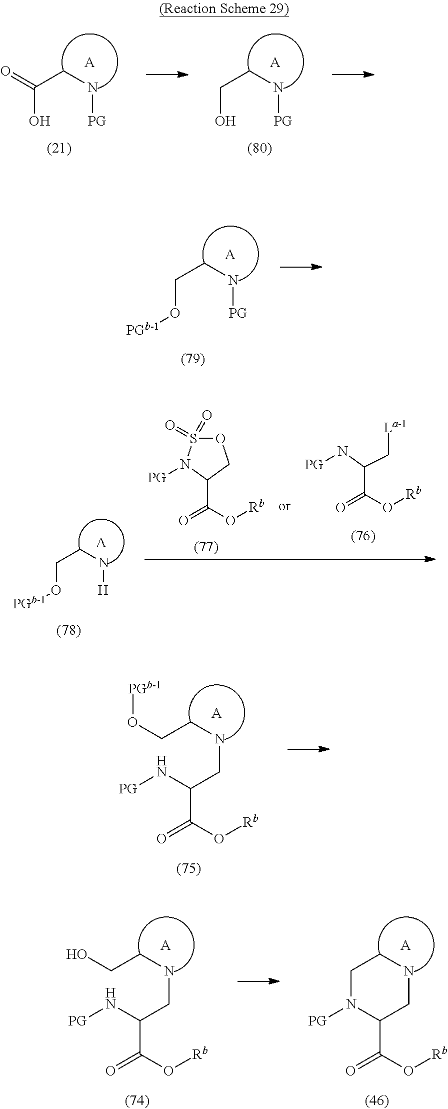

wherein PG^{b-1} is a hydroxy-protecting group, and the other symbols are each as defined above.

Examples of the hydroxy-protecting group for PG^{b-1} include those exemplified as the below-mentioned hydroxy-protecting group.

Compound (80) can be produced by subjecting compound (21) to a reduction reaction known per se (e.g., the methods described in Comprehensive Organic Transformations, John Wiley and Sons (1999); EP 1428824; EP 1553074; US 2003/78249; U.S. Pat. No. 5,071,844; U.S. Pat. No. 4,539,411 and the like) to convert the carboxylic acid into the primary alcohol.

The reduction reaction is carried out, for example, using a reducing agent. Examples of the reducing agent include borane-dimethylsulfide complex, borane-tetrahydrofuran complex, borane-ether complex, diborane and lithium aluminum hydride.

The amount of the reducing agent to be used is generally 1 to 1000 equivalents, preferably 1 to 20 equivalents, per 1 equivalent of compound (21).

This reaction is preferably carried out in a solvent inert to the reaction. The solvent is not particularly limited as long as the reaction proceeds, and examples thereof include ethers.

The reaction temperature for this reaction is generally −78° C. to 200° C. The reaction time is generally 0.5 to 100 hr.

Compound (79) can be produced by introducing the protecting group PG^{b-1} into compound (80) according to a reaction known per se (e.g., the method described in Protective Groups in Organic Synthesis, John Wiley and Sons (1980)).

Compound (78) can be produced by removing the PG of compound (79) in the same manner as in the production method of compound (2a) from compound (4a) in Reaction Scheme 2.

Compound (75) can be produced by subjecting compound (78) and compound (77) to a reaction known per se (e.g., the method described in Synlett, 2008, 5, 702-706).

The amount of compound (77) to be used is generally 1 to 1000 equivalents, preferably 1 to 20 equivalents, per 1 equivalent of compound (78).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (75) can also be produced by subjecting compound (78) and compound (76) to a reaction known per se (e.g., the method described in WO 2005/58873; WO 2006/134341 and the like).

The amount of compound (76) to be used is generally 1 to 1000 equivalents, preferably 1 to 20 equivalents, per 1 equivalent of compound (78).

This reaction is preferably carried out in a solvent inert to the reaction. The solvent is not particularly limited as long as the reaction proceeds, and examples thereof include alcohols, ethers, esters, halogenated hydrocarbons, hydrocarbons, amides, nitriles, sulfoxides, sulfolane and hexamethylphosphoramide. These solvents may be used in a mixture of two or more kinds thereof at an appropriate ratio.

The reaction temperature for this reaction is generally −78° C. to 200° C. The reaction time is generally 0.5 to 100 hr.

Compound (76) may be commercially available, or can be synthesized according to a method known per se (e.g., the method described in Tetrahedron: Asymmetry, 1990, 1, 881-884 and the like).

Compound (77) may be commercially available, or can be synthesized according to a method known per se (e.g., the method described in WO2006/55725; WO2005/44817; U.S. Pat. No. 4,795,815 and the like).

Compound (74) can be produced by removing the PG^{b-1} of compound (75) in the same manner as in the production method of compound (2a) from compound (4a) in Reaction Scheme 2.

Compound (46) can be produced, for example, by converting the hydroxy group of compound (74) into a leaving group, and subjecting the resulting compound to an intramolecular cyclization reaction according to a reaction known per se (e.g., the methods described in Synlett, 2008, 5, 702-706; Bioorg. Med. Chem. Lett., 2003, 13, 3243-3246).

Furthermore, a compound encompassed in the present invention can also be produced by applying a means known per se to compound (I) for further introduction of substituent or functional group conversion.

The conversion of substituent is performed by a conventional known method. For example, conversion to carboxy by ester hydrolysis, conversion to carbamoyl by amidation of carboxy, conversion to hydroxymethyl by reduction of carboxy, conversion to alcohol form by reduction of carbonyl or addition reaction of alkyl to carbonyl, reductive amination of carbonyl, oximation of carbonyl, acylation of amino, ureation of amino, sulfonylation of amino, alkylation of amino, substitution or amination of active halogen by amine, alkylation of hydroxy, substitution or amination of hydroxy and the like can be mentioned.

When substituent introduction or functional group conversion is to be performed and a reactive moiety possibly causing a reaction other than desired is present, the reactive moiety may be protected as necessary by a protecting group in advance according to a means known per se, and the protecting group may be removed by a means known per se after the desired reaction, whereby a compound encompassed in the present invention can be produced.

For example, when the starting compound or an intermediate have amino, carboxy or hydroxy as a substituent, these groups may be protected by a protecting group generally used in the peptide chemistry and the like. In this case, an object compound can be obtained by removing the protecting group as necessary after the reaction.

Examples of the amino-protecting group include those exemplified as PG in the above.

Examples of the carboxy-protecting group include C₁₋₆ alkyl, C₇₋₁₁ aralkyl (e.g., benzyl), phenyl, trityl, substituted silyl (e.g., trimethylsilyl, triethylsilyl, dimethylphenylsilyl, tert-butyldimethylsilyl, tert-butyldiethylsilyl) and C₂₋₆ alkenyl (e.g., 1-allyl).

Examples of the hydroxy-protecting group include C₁₋₆ alkyl, phenyl, trityl, C₇₋₁₀ aralkyl (e.g., benzyl), formyl, C₁₋₆ alkyl-carbonyl, benzoyl, C₇₋₁₀ aralkyl-carbonyl (e.g., benzylcarbonyl), 2-tetrahydropyranyl, 2-tetrahydrofuranyl, substituted silyl (e.g., trimethylsilyl, triethylsilyl, dimethylphenylsilyl, tert-butyldimethylsilyl, tert-butyldiethylsilyl) and C₂₋₆ alkenyl (e.g., 1-allyl).

These groups are optionally substituted by 1 to 3 substituents selected from a halogen atom, C₁₋₆ alkyl, C₁₋₆ alkoxy and nitro.

The above-mentioned protecting group can be removed by a method known per se, for example, the method described in Protective Groups in Organic Synthesis, John Wiley and Sons (1980) and the like. Specifically, a method using acid, base, ultraviolet rays, hydrazine, phenylhydrazine, sodium N-methyldithiocarbamate, tetrabutylammonium fluoride, palladium acetate, trialkylsilylhalide (e.g., trimethylsilyliodide, trimethylsilylbromide etc.) and the like, a reduction method and the like are used.

Depending on the kind of the substituent of the starting compound, a starting compound having a different substituent can be produced by the above-mentioned substituent conversion using the compound produced by the above-mentioned production method as a starting material.

Compound (I), which is the resultant product of this reaction, may be produced as a single compound or a mixture.

The thus-obtained compound (I) can be isolated and purified by a separation means known per se, for example, concentration, concentration under reduced pressure, solvent extraction, crystallization, recrystallization, phase transfer and chromatography.

When compound (I) is obtained as a free compound, it can be converted to a desired salt by a method known per se or a method analogous thereto. Conversely, when the compound is obtained as a salt, it can be converted to a free form or other desired salt by a method known per se or a method analogous thereto.

When compound (I) has an isomer such as optical isomer, stereoisomer, positional isomer and the like, any one isomer and a mixture thereof are also encompassed in compound (I). For example, when an optical isomer is present in compound (I), an optical isomer resolved from a racemate is also encompassed in compound (I). These isomers can be obtained as single products by synthesis method and separation method (e.g., concentration, solvent extraction, column chromatography, recrystallization) known per se.

Compound (I) may be a crystal, and both single crystal form and a crystalline mixture are encompassed in compound (I). The crystal can be produced by crystallization by a crystallization method known per se.

Compound (I) may be a hydrate, a non-hydrate, a solvate, or a non-solvate.

Compound (I) also encompasses a compound labeled with an isotope (e.g., ³H, ¹⁴C, ³⁵S, ¹²⁵I etc.) and the like.

Furthermore, compound (I) may also be a deuterium converter.

A prodrug of compound (I) or a salt thereof means a compound converted to compound (I) by a reaction due to an enzyme, a gastric acid, etc. under the physiological condition in the living body, that is, a compound converted to compound (I) by oxidation, reduction, hydrolysis, etc. due to an enzyme, a compound converted to compound (I) by hydrolysis etc. due to gastric acid, and the like. A prodrug of compound (I) can be used a compound obtained by subjecting an amino in compound (I) to an acylation, alkylation or phosphorylation (e.g., a compound obtained by subjecting an amino in compound (I) to eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation); a compound obtained by subjecting hydroxy in compound (I) to acylation, alkylation, phosphorylation or boration (e.g., a compound obtained by subjecting hydroxy in compound (I) to acetylation, palmitoylation, propanoylation, pivaloylation, succinylation, fumarylation, alanylation or dimethylaminomethylcarbonylation); a compound obtained by subjecting carboxy in compound (I) to esterification or amidation (e.g., a compound obtained by subjecting carboxy in compound (I) to ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any one of these compounds can be produced from compound (I) by a method known per se.

A prodrug of compound (I) may also be a compound converted into compound (I) under physiological conditions, such as those described in IYAKUHIN no KAIHATSU (Development of Pharmaceuticals), Vol. 7, Design of Molecules, p. 163-198, Published by HIROKAWA SHOTEN (1990).

Compound (I) or a prodrug thereof (sometimes to be abbreviated as “the compound of the present invention” in the present specification) has an IAP antagonistic (inhibitory) activity, and is useful as a prophylactic or therapeutic agent clinically useful for cancer, growth inhibitor of cancer, cancer metastasis suppressant, apoptosis promoter and the like.

In addition, the compound of the present invention can be used for the prophylaxis or treatment of IAP-associated diseases in mammal.

The compound of the present invention shows a strong antagonistic activity against IAP, particularly, XIAP (X chromosome linked Inhibitor of Apoptosis Protein), cIAP1 (cellular Inhibitor of Apoptosis Protein 1), cIAP2 (cellular Inhibitor of Apoptosis Protein 2) and the like.

Since the compound of the present invention is also superior in the efficacy expression, pharmacokinetics (absorption, distribution, metabolism, excretion etc.), solubility (water-solubility etc.), interaction with other pharmaceutical products, safety (low in the toxicity such as acute toxicity, chronic toxicity, genetic toxicity, reproductive toxicity, cardiotoxicity, carcinogenicity etc.), stability (chemical stability, stability to enzyme etc.) and the like, it is useful as a medicament.

Therefore, the compound of the present invention is useful for mammals (e.g., mouse, rat, hamster, rabbit, cat, dog, bovine, sheep, monkey, human etc.) as an IAP (particularly, XIAP and/or cIAP (above-mentioned cIAP1 and cIAP2)) antagonist.

Accordingly, the compound of the present invention is used as a medicament such as a prophylactic or therapeutic agent for IAP-associated disease, for example, cancer [e.g., colorectal cancer (e.g., familial colorectal cancer, hereditary nonpolyposis colorectal cancer, gastrointestinal stromal tumor, etc.), lung cancer (e.g., non-small cell lung cancer, small cell lung cancer, malignant mesothelioma, etc.), mesothelioma, pancreatic cancer (e.g., pancreatic duct cancer, etc.), gastric cancer (e.g., papillary adenocarcinoma, mucinous adenocarcinoma, adenosquamous cancer, etc.), breast cancer (e.g., invasive ductal carcinoma, ductal cancer in situ, inflammatory breast cancer, etc.), ovarian cancer (e.g., ovarian epithelial cancer, extragonadal germ cell tumor, ovarian germ cell tumor, ovarian low malignant potential tumor, etc.), prostate cancer (e.g., hormone-dependent prostate cancer, non-hormone dependent prostate cancer, etc.), liver cancer (e.g., primary liver cancer, extrahepatic bile duct cancer, etc.), thyroid cancer (e.g., medullary thyroid cancer, etc.), kidney cancer (e.g., renal cell carcinoma, renal pelvis and ureter transitional cell cancer, etc.), uterine cancer, brain tumor (e.g., pineal astrocytoma, pilocytic astrocytoma, diffuse astrocytoma, anaplastic astrocytoma, etc.), melanoma, sarcoma, urinary bladder cancer, hematologic cancer (e.g., multiple myeloma)] and the like; growth inhibitor of cancer; cancer metastasis suppressant; apoptosis promoter; and the like.

Particularly, the compound of the present invention is effective for breast cancer, ovarian cancer, pancreatic cancer, lung cancer, hematologic cancer and the like.

The compound of the present invention can be administered orally or parenterally as it is or in a mixture with a pharmacologically acceptable carrier as medicament.

The dosage form of the compound of the present invention for oral administration is, for example, an oral preparation such as tablet (including sugar-coated tablet, film-coated tablet, sublingual tablet, buccal tablet, mouth cavity quick-integrating tablet), pill, granule, powder, capsule (including soft capsule, microcapsule), syrup, emulsion, suspension, films (e.g., mouth cavity mucous membrane adhesion film) and the like, and the dosage form for parenteral administration is, for example, injection, injecting agent, instillation and suppository. In addition, it is effective to make a sustained release preparation by combining the compound with a suitable base (e.g., polymer of butyric acid, polymer of glycolic acid, copolymer of butyric acid-glycolic acid, a mixture of a polymer of butyric acid and a polymer of glycolic acid, polyglycerol fatty acid ester etc.).

As a method for producing the compound of the present invention in the above-mentioned dosage form, a known production method (e.g., the method described in the Japanese Pharmacopoeia) generally used in the pertinent field can be employed. When the above-mentioned dosage form is produced, suitable amounts of additives such as excipient, binder, disintegrant, lubricant, sweetening agent, surfactant, suspending agent, emulsifier and the like generally used in the pharmaceutical field, are appropriately added as necessary for production.

When the compound of the present invention is prepared into a tablet, for example, it can be produced by adding an excipient, a binder, a disintegrant, a lubricant and the like, and when a pill or a granule is to be prepared, it can be produced by adding an excipient, a binder, a disintegrant and the like. When a powder or a capsule is to be prepared, it can be produced by adding an excipient and the like, when a syrup is to be prepared, it can be produced by adding a sweetener and the like, and when an emulsion or a suspension is to be prepared, it can be produced by adding a suspending agent, a surfactant, an emulsifier and the like.

Examples of the excipient include lactose, sucrose, glucose, starch, sucrose, crystalline cellulose, powdered glycyrrhiza, mannitol, sodium hydrogen carbonate, calcium phosphate and calcium sulfate.

Examples of the binder include 5 to 10 wt % starch liquid paste, 10 to 20 wt % gum arabic solution or gelatin solution, 1 to 5 wt % tragacanth solution, carboxymethyl cellulose solution, sodium alginate solution and glycerin.

Examples of the disintegrant include starch and calcium carbonate.

Examples of the lubricant include magnesium stearate, stearic acid, calcium stearate and purified talc.

Examples of the sweetener include glucose, fructose, invert sugar, sorbitol, xylitol, glycerin and simple syrup.

Examples of the surfactant include sodium lauryl sulfate, polysorbate 80, sorbitan monofatty acid ester and polyoxyl 40 stearate.

Examples of the suspending agent include gum arabic, sodium alginate, sodium carboxymethyl cellulose, methyl cellulose and bentonite.

Examples of the emulsifier include gum arabic, tragacanth, gelatin and polysorbate 80.

Furthermore, when the compound of the present invention is produced in the above-mentioned dosage form, a suitable amount of a colorant, a preservative, an aromatic, a corrigent, a stabilizer, a thickening agent and the like typically used in the field of preparation can be added on demand.

As the above-mentioned injection, intravenous injection as well as subcutaneous injection, intracutaneous injection, intramuscular injection, instillation and the like are mentioned, and as the sustained release preparation, an iontophoresis transdermal agent and the like are mentioned.

Such injections are prepared by methods known per se, or by dissolving, suspending or emulsifying the compound of the present invention in a sterilized aqueous or oily liquid. As an aqueous liquid for injection, physiological saline, isotonic solutions containing glucose or other auxiliary drugs (e.g., D-sorbitol, D-mannitol, sodium chloride) and the like, and they can be used in combination with suitable solubilizing agents, such as alcohols (e.g., ethanol), polyalcohols (e.g., propylene glycol, polyethylene glycol), nonionic surfactants (e.g., polysorbate 80, HCO-50) and the like. As an oily liquid, sesame oil, soybean oil and the like, which may be used in combination with solubilizing agents such as benzyl benzoate, benzyl alcohol and the like. In addition, buffers (e.g., phosphate buffer, sodium acetate buffer), soothing agents (e.g., benzalkonium chloride, procaine hydrochloride), stabilizers (e.g., human serum albumin, polyethylene glycol), preservatives (e.g., benzyl alcohol, phenol) and the like can be blended. A prepared injection is generally filled in an ampoule.

While the content of the compound of the present invention in the medicament of the present invention (specifically the above-mentioned various dosage form) varies depending on the form of the pharmaceutical preparation, it is generally about 0.01 to 100 wt %, preferably about 2 to 85 wt %, more preferably about 5 to 70 wt %, relative to the entire preparation.

While the content of the additive in the medicament of the present invention varies depending on the form of the pharmaceutical preparation, it is generally about 1 to 99.9 wt %, preferably about 10 to 90 wt %, relative to the entire preparation.

The compound of the present invention is stable and low toxic, and can be used safely. While the daily dose varies depending on the condition and body weight of patients, the kind of compound, administration route and the like, in the case of, for example, oral administration to patients for the treatment of cancer, the daily dose to an adult (body weight about 60 kg) is about 1 to 2000 mg, preferably about 3 to 1000 mg, more preferably about 10 to 250 mg, as an active ingredient (the compound of the present invention), which can be given in a single administration or administered in 2 or 3 portions a day.

When the compound of the present invention is administered parenterally, it is generally administered in the form of a liquid (e.g., injection). While the dose varies depending on the subject of administration, target organ, symptom, administration method and the like, it is, for example, about 0.01 to about 100 mg, preferably about 0.01 to about 50 mg, more preferably about 0.01 to about 20 mg, in the form of an injection, relative to kg body weight, which is preferably given by intravenous injection or drip.

The compound of the present invention can be used concurrently with other drugs. Specifically, the compound of the present invention can be used together with medicaments such as hormonal therapeutic agents, chemotherapeutic agents, immunotherapeutic agents, medicaments inhibiting the action of cell growth factors or cell growth factor receptors and the like. In the following, the drugs that can be used in combination with the compound of the present invention are abbreviated as “concomitant drugs”.

Examples of the “hormonal therapeutic agents” include fosfestrol, diethylstylbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, anti-estrogens (e.g., tamoxifen citrate, toremifene citrate), pill preparations, mepitiostane, testrolactone, aminoglutethimide, LH-RH agonists (e.g., goserelin acetate, buserelin, leuprorelin), droloxifene, epitiostanol, ethinylestradiol sulfonate, aromatase inhibitors (e.g., fadrozole hydrochloride, anastrozole, retrozole, exemestane, vorozole, formestane), anti-androgens (e.g., flutamide, bicartamide, nilutamide), 5α-reductase inhibitors (e.g., finasteride, epristeride), aderenal cortex hormone drugs (e.g., dexamethasone, prednisolone, betamethasone, triamcinolone), androgen synthesis inhibitors (e.g., abiraterone), retinoid and drugs that retard retinoid metabolism (e.g., liarozole), thyroid gland hormone and DDS (Drug Delivery System) preparations thereof.

Examples of the “chemotherapeutic agents” include alkylating agents, antimetabolites, anticancer antibiotics and plant-derived anticancer agents.

Examples of the “alkylating agents” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin and DDS (Drug Delivery System) preparations thereof.

Examples of the “antimetabolites” include mercaptopurine, 6-mercaptopurine riboside, thioinosine, methotrexate, pemetrexed, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, capecitabine), aminopterine, nelzarabine, leucovorin calcium, tabloid, butocine, calcium folinate, levofolinate calcium, cladribine, emitefur, fludarabine, gemcitabine, hydroxycarbamide, pentostatin, piritrexim, idoxuridine, mitoguazone, thiazophrine, ambamustine, bendamustine and DDS preparations thereof.

Examples of the “anticancer antibiotics” include actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride and DDS preparations thereof.

Examples of the “plant-derived anticancer agents” include etoposide, etoposide phosphate, vinblastine sulfate, vincristine sulfate, vindesine sulfate, teniposide, paclitaxel, docetaxel, vinorelbine and DDS preparations thereof.

Examples of the “immunotherapeutic agents” include picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole and anti-CTLA4 antibody.

Example of the “cell growth factors” in the “medicaments inhibiting the action of cell growth factors or cell growth factor receptors” include any substances that promote cell proliferation, which are normally peptides having not more than 20,000 molecular weight that are capable of exhibiting their activity at low concentrations by binding to a receptor, including (1) EGF (epidermal growth factor) or substances possessing substantially the same activity as EGF [e.g., TGFα,],

(2) insulin or substances possessing substantially the same activity as insulin [e.g., insulin, IGF (insulin-like growth factor)-1, IGF-2], (3) FGF (fibroblast growth factor) or substances possessing substantially the same activity as FGF [e.g., acidic FGF, basic FGF, KGF (keratinocyte growth factor), FGF-10], and (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), heregulin, angiopoietin, and the like].

Examples of the “cell growth factor receptors” include any receptors capable of binding to the above-mentioned cell growth factors, including EGF receptor, heregulin receptor (e.g., HER3), insulin receptor, IGF receptor-1, IGF receptor-2, FGF receptor-1 or FGF receptor-2, VEGF receptor, angiopoietin receptor (e.g., Tie2), PDGF receptor, TNFα receptor and the like.

As the “medicaments inhibiting the action of cell growth factors or cell growth factor receptors”, for example, EGF inhibitor, TGFα inhibitor, heregulin inhibitor, insulin inhibitor, IGF inhibitor, FGF inhibitor, KGF inhibitor, CSF inhibitor, EPO inhibitor, IL-2 inhibitor, NGF inhibitor, PDGF inhibitor, TGFβ inhibitor, HGF inhibitor, VEGF inhibitor, angiopoietin inhibitor, EGF receptor inhibitor, HER2 inhibitor, HER4 inhibitor, insulin receptor inhibitor, IGF-1 receptor inhibitor, IGF-2 receptor inhibitor, FGF receptor-1 inhibitor, FGF receptor-2 inhibitor, FGF receptor-3 inhibitor, FGF receptor-4 inhibitor, VEGF receptor inhibitor, Tie-2 inhibitor, PDGF receptor inhibitor, Abl inhibitor, Raf inhibitor, FLT3 inhibitor, c-Kit inhibitor, Src inhibitor, PKC inhibitor, Trk inhibitor, Ret inhibitor, mTOR inhibitor, Aurora inhibitor, PLK inhibitor, MEK (MEK1/2) inhibitor, MET inhibitor, CDK inhibitor, Akt inhibitor, ERK inhibitor and the like are used. As such medicament, more specifically, anti-VEGF antibody (e.g., Bevacizumab), anti-HER2 antibody (e.g., Trastuzumab, Pertuzumab), anti-EGFR antibody (e.g., Cetuximab, Panitumumab, Matuzumab, Nimotuzumab), anti-VEGFR antibody, anti-HGF antibody, Imatinibmesylate, Erlotinib, Gefitinib, Sorafenib, Sunitinib, Dasatinib, Lapatinib, Vatalanib, 4-(4-fluoro-2-methyl-1H-indol-5-yloxy)-6-methoxy-7-[3-(1-pyrrolidinyl)propoxy]quinazoline (AZD-2171), Lestaurtinib, Pazopanib, Canertinib, Tandutinib, 3-(4-bromo-2,6-difluorobenzyloxy)-5-[3-[4-(1-pyrrolidinyl)butyl]ureido]isothiazole-4-carboxamide (CP-547632), Axitinib, N-(3,3-dimethyl-2,3-dihydro-1H-indol-6-yl)-2-(pyridin-4-ylmethylamino)pyridine-3-carboxamide (AMG-706), Nilotinib, 6-[4-(4-ethylpiperazin-1-ylmethyl)phenyl]-N-[1(R)-phenylethyl]-7H-pyrrolo[2,3-d]pyrimidin-4-amine (AEE-788), Vandetanib, Temsirolimus, Everolimus, Enzastaurin, N-[4-[4-(4-methylpiperazin-1-yl)-6-(3-methyl-1H-pyrazol-5-ylamino)pyrimidin-2-ylsulfanyl]phenyl]cyclopropanecarboxamide (VX-680), 2-[N-[3-[4-[5-[N-(3-fluorophenyl)carbamoylmethyl]-1H-pyrazol-3-ylamino]quinazolin-7-yloxy]propyl]-N-ethylamino]ethyl phosphate (AZD-1152), 4-[9-chloro-7-(2,6-difluorophenyl)-5H-pyrimido[5,4-d][2]benzazepin-2-ylamino]benzoic acid (MLN-8054), N-[2-methoxy-5-[(E)-2-(2,4,6-trimethoxyphenyl)vinylsulfonylmethyl]phenyl]glycine sodium salt (ON-1910Na), 4-[8-cyclopentyl-7(R)-ethyl-5-methyl-6-oxo-5,6,7,8-tetrahydropteridin-2-ylamino]-3-methoxy-N-(1-methylpiperidin-4-yl)benzamide (BI-2536), 5-(4-bromo-2-chlorophenylamino)-4-fluoro-1-methyl-1H-benzimidazole-6-carbohydroxamic acid 2-hydroxyethyl ester (AZD-6244), N-[2(R),3-dihydroxypropoxy]-3,4-difluoro-2-(2-fluoro-4-iodophenylamino)benzamide (PD-0325901) and Everolimus (RAD001) are used.

In addition to the above-mentioned drugs, L-asparaginase, aceglatone, procarbazine hydrochloride, protoporphyrin-cobalt complex salt, mercuric hematoporphyrin-sodium, topoisomerase I inhibitors (e.g.; irinotecan, topotecan), topoisomerase II inhibitors (e.g., sobuzoxane), differentiation inducers (e.g., retinoid, vitamin D), other angiogenesis inhibitors (e.g., humagillin, shark extract, COX-2 inhibitor), α-blockers (e.g., tamsulosin hydrochloride), bisphosphonic acids (pamidronate, zoledronate), thalidomide, 5-azacytidine, decitabine, proteasome inhibitors (e.g., bortezomib), agent for inducing apoptosis (e.g., TRAIL receptor agonist, anti-TRAIL antibody, Bcl-2 inhibitor), antitumor antibodies (e.g., anti-CD20 antibody), toxin labeled antibody and the like can be also used as concomitant drug.

By combining the compound of the present invention and a concomitant drug, a superior effect such as

(1) the dose can be reduced as compared to single administration of the compound of the present invention or a concomitant drug,
(2) the drug to be combined with the compound of the present invention can be selected according to the condition of patients

(mild case, severe case and the like),

(3) the period of treatment can be set longer,
(4) a sustained treatment effect can be designed,
(5) a synergistic effect can be afforded by a combined use of the compound of the present invention and a concomitant drug, and the like, can be achieved.

In the present specification, the compound of the present invention and a concomitant drug used in combination are referred to as the “combination agent of the present invention”.

For use of the combination agent of the present invention, the administration time of the compound of the present invention and the concomitant drug is not restricted, and the compound of the present invention and the concomitant drug can be administered to an administration subject simultaneously, or may be administered at different times. The dosage of the concomitant drug may be determined according to the dose clinically set, and can be appropriately selected depending on the administration subject, administration route, disease, combination and the like.

Examples of the administration mode of the combined use of the compound of the present invention and the concomitant drug include the following methods: (1) The compound of the present invention and the concomitant drug are simultaneously produced to give a single preparation, which is then administered. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route at different times. (4) The compound of the present invention and the concomitant drug are separately produced to give two kinds of preparations which are administered simultaneously by different administration routes. (5) The compound of the present invention and the concomitant drug are separately produced to give two kinds of preparations which are administered by different administration routes at different times (e.g., the compound of the present invention and the concomitant drug are administered in this order, or in the reverse order).

The dose of the concomitant drug is appropriately determined in accordance with its clinical dose, and the ratio of the compound of the present invention and the concomitant drug is appropriately determined depending on the administration subject, administration route, target disease, symptom, combination, and the like. For example, when the administration subject is human, the concomitant drug is used in 0.01 to 100 (parts by weight), relative to 1 part by weight of the compound of the present invention.

The combination agent of the present invention has low toxicity and, for example, the compound of the present invention and/or the above-mentioned concomitant drug can be mixed, according to a method known per se, with a pharmacologically acceptable carrier to give pharmaceutical compositions, such as tablets (including sugar-coated tablet, film-coated tablet), powders, granules, capsules (including soft capsule), solutions, injections, suppositories, sustained release agents and the like, which can be safely administered orally or parenterally (e.g., local, rectum, venous). An injection can be administered by intravenous, intramuscular, subcutaneous or intra-tissue administration, or directly to the lesion.

As a pharmacologically acceptable carrier which may be used for preparing the combination agent of the present invention, those similar to the above-mentioned pharmacologically acceptable carriers, that can be used for the production of the medicament of the present invention, can be mentioned. Where necessary, the above-mentioned additives that can be used for the production of the medicament of the present invention, such as preservatives, antioxidants, colorants, sweetening agents, adsorbents, wetting agents and the like can also be appropriately used in appropriate amounts.

The compounding ratio of the compound of the present invention to the concomitant drug in the combination agent of the present invention can be appropriately set depending on the administration subject, administration route, diseases and the like.

For example, the content of the compound of the present invention in the combination agent of the present invention varies depending on the dosage form, and is usually from about 0.01 to 100% by weight, preferably from about 0.1 to 50% by weight, further preferably from about 0.5 to 20% by weight, based on the entire preparation.

The content of the concomitant drug in the combination agent of the present invention varies depending on the dosage form, and is usually from about 0.01 to 90% by weight, preferably from about 0.1 to 50% by weight, further preferably from about 0.5 to 20% by weight, based on the entire preparation.

The content of additives in the combination agent of the present invention varies depending on the dosage form, and is usually from about 1 to 99.99% by weight, preferably from about 10 to 90% by weight, based on the entire preparation.

When the compound of the present invention and the concomitant drug are separately prepared, the same content may be adopted.

These preparations can be produced by a method known per se, which is generally employed in the preparation process.

For example, the compound of the present invention and the concomitant drug can be made into an aqueous injection together with a dispersing agent (e.g., Tween 80 (manufactured by Atlas Powder, US), HCO 60 (manufactured by Nikko Chemicals), polyethylene glycol, carboxymethylcellulose, sodium alginate, hydroxypropylmethylcellulose, dextrin), a stabilizer (e.g., ascorbic acid, sodium pyrosulfite), a surfactant (e.g., Polysorbate 80, macrogol), a solubilizer (e.g., glycerin, ethanol), a buffer (e.g., phosphoric acid and alkali metal salt thereof, citric acid and alkali metal salt thereof), an isotonizing agent (e.g., sodium chloride, potassium chloride, mannitol, sorbitol, glucose), a pH adjuster (e.g., hydrochloric acid, sodium hydroxide), a preservative (e.g., ethyl paraoxybenzoate, benzoic acid, methylparaben, propylparaben, benzyl alcohol), a dissolving agent (e.g., conc. glycerin, meglumine), a solubilizing agent (e.g., propylene glycol, sucrose), a soothing agent (e.g., glucose, benzyl alcohol), and the like, or can be dissolved, suspended or emulsified in a vegetable oil such as olive oil, sesame oil, cotton seed oil, corn oil and the like or a solubilizing agent such as propylene glycol and the like and prepared into an oily injection, whereby an injection is afforded.

In addition, an excipient (e.g., lactose, sucrose, starch), a disintegrating agent (e.g., starch, calcium carbonate), a binder (e.g., starch, gum arabic, carboxymethylcellulose, polyvinylpyrrolidone, hydroxypropylcellulose) or a lubricant (e.g., talc, magnesium stearate, polyethylene glycol 6000) may be added to the compound of the present invention or the concomitant drug, and the mixture can be compression-molded, according to a method known per se then if desirable, the molded product can be coated by a method known per se for the purpose of masking of taste, enteric property or durability, to give a preparation for oral administration. As the coating agent, for example, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudoragit (methacrylic acid•acrylic acid copolymer, manufactured by Rohm, DE), and pigment (e.g., iron oxide red, titanium dioxide) can be used. The preparation for oral administration may be any of an immediate-release preparation and a sustained release preparation.

Moreover, the compound of the present invention and the concomitant drug can be made into an oily or aqueous solid, semisolid or liquid suppository according to a method known per se, by mixing them with an oily substrate, aqueous substrate or aqueous gel substrate.

As the oily substrate, for example, glycerides of higher fatty acid [e.g., cacao butter, Witepsols (manufactured by Dynamit Nobel, Germany)], glycerides of medium chain fatty acid [e.g., Miglyols (manufactured by Dynamit Nobel, Germany)] and vegetable oils (e.g., sesame oil, soybean oil, cotton seed oil) are mentioned.

As the aqueous substrate, for example, polyethylene glycol and propylene glycol are mentioned, and as the aqueous gel substrate, for example, natural gums, cellulose derivatives, vinyl polymers and acrylic acid polymers are mentioned.

As the above-mentioned sustained release preparation, sustained release microcapsules and the like are mentioned. The sustained release microcapsule can be produced by a method known per se, for example, a method shown in the following [2].

The compound of the present invention is preferably molded into a preparation for oral administration such as a solid preparation (e.g., powder, granule, tablet, capsule) and the like, or molded into a preparation for rectal administration such as a suppository and the like. Particularly, a preparation for oral administration is preferable.

The concomitant drug can be made into the above-mentioned drug form depending on the kind of the drug.

[1] An injection of the compound of the present invention or the concomitant drug, and preparation thereof, [2] a sustained release preparation or immediate-release preparation of the compound of the present invention or the concomitant drug, and preparation thereof, [3] a sublingual, buccal or intraoral quick integrating agent of the compound of the present invention or the concomitant drug, and preparation thereof, will be described below specifically.

### [1] Injection and Preparation Thereof.

An injection prepared by dissolving the compound of the present invention or the concomitant drug into water is preferable. This injection may be allowed to contain a benzoate and/or salicylate.

The injection is obtained by dissolving the compound of the present invention or the concomitant drug, and if desirable, a benzoate and/or salicylate, into water.

As the above-mentioned salts of benzoic acid and salicylic acid, for example, salts of alkali metals such as sodium, potassium and the like, salts of alkaline earth metals such as calcium, magnesium and the like, ammonium salts, meglumine salts, salts with organic bases such as tromethamol and the like are listed.

The concentration of the compound of the present invention or the concomitant drug in an injection is from 0.5 to 50 w/v %, preferably from about 3 to 20 w/v %. The concentration of a benzoate or/and salicylate is from 0.5 to 50 w/v %, preferably from about 3 to 20 w/v %.

Into the injection of the present invention, additives usually used in an injection, for example, a stabilizer (e.g., ascorbic acid, sodium pyrosulfite), a surfactant (e.g., Polysorbate 80, macrogol), a solubilizer (e.g., glycerin, ethanol), a buffer (e.g., phosphoric acid and alkali metal salt thereof, citric acid and alkali metal salt thereof), an isotonizing agent (e.g., sodium chloride, potassium chloride), a dispersing agent (e.g., hydroxypropylmethylcellulose, dextrin), a pH regulator (e.g., hydrochloric acid, sodium hydroxide), a preservative (e.g., ethyl paraoxybenzoate, benzoic acid), a dissolving agent (e.g., conc. glycerin, meglumine), a solubilizing agent (e.g., propylene glycol, sucrose), a soothing agent (e.g., glucose, benzyl alcohol) can be appropriately blended. These additives are generally blended in a proportion usually used in an injection.

It is advantageous that pH of an injection is controlled from pH 2 to 12, preferably from pH 2.5 to 8.0 by addition of a pH regulator.

An injection is obtained by dissolving the compound of the present invention or the concomitant drug and if desirable, a benzoate and/or a salicylate, and if necessary, the above-mentioned additives into water. These may be dissolved in any order, and can be appropriately dissolved in the same manner as in a conventional method of producing an injection.

An aqueous solution for injection may be advantageously heated, alternatively, for example, filter sterilization, high pressure heat sterilization and the like can be conducted in the same manner as for a usual injection, to provide an injection.

It may be advantageous that an aqueous solution for injection is subjected to high pressure heat sterilization at 100 to 121° C. for 5 to 30 min.

Further, a preparation endowed with an antibacterial property of a solution may also be produced so that it can be used as a preparation which is divided and administered multiple-times.

### [2] Sustained Release Preparation or Immediate-Release Preparation, and Preparation Thereof

A sustained release preparation is preferable which is obtained, if desirable, by coating a nucleus containing the compound of the present invention or the concomitant drug with a film agent such as a water-insoluble substance, swellable polymer and the like. For example, a sustained release preparation for oral administration of once administration per day type is preferable.

As the water-insoluble substance used in a film agent, there are listed, for example, cellulose ethers such as ethylcellulose, butylcellulose and the like, cellulose esters such as cellulose acetate, cellulose propionate and the like, polyvinyl esters such as polyvinyl acetate, polyvinyl butyrate and the like, acrylic acid/methacrylic acid copolymers, methyl methacrylate copolymers, ethoxyethyl methacrylate/cinnamoethyl methacrylate/aminoalkyl methacrylate copolymers, polyacrylic acid, polymethacrylic acid, methacrylic acid alkylamide copolymers, poly(methyl methacrylate), polymethacrylate, polymethacrylamide, aminoalkyl methacrylate copolymers, poly(methacrylic anhydride), glycidyl methacrylate copolymer, particularly, acrylic acid-based polymers such as Eudoragit (manufactured by Rohm Pharma) such as Eudoragit RS-100, RL-100, RS-30D, RL-30D, RL-PO, RS-PO (ethyl acrylate/methyl methacrylate/trimethylchloride methacrylate/ethyl ammonium copolymer), Eudoragit NE-30D (methyl methacrylate/ethyl acrylate copolymer), and the like, hydrogenated oils such as hydrogenated castor oil (e.g., Lubri wax (manufactured by Freund Corporation) and the like), waxes such as carnauba wax, fatty acid glycerin ester, paraffin and the like, and polyglycerol ester of fatty acids.

As the swellable polymer, polymers having an acidic dissociating group and showing pH dependent swell are preferable, and polymers having an acidic dissociating group, which manifest small swelling in acidic regions such as in stomach and large swelling in neutral regions such as in small intestine and large intestine, are preferable.

As such a polymer having an acidic dissociating group and showing pH dependent swell, cross-linkable polyacrylic acid polymers such as, Carbomer 934P, 940, 941, 974P, 980, 1342 and the like, polycarbophil, calcium polycarbophil (all are manufactured by BF Goodrich), Hiviswako 103, 104, 105, 304 (all are manufactured by Wako Pure Chemical Industries, Ltd.), and the like, are listed.

The film agent used in a sustained release preparation may further contain a hydrophilic substance.

As the hydrophilic substance, for example, polysaccharides which may contain a sulfate group such as pullulan, dextrin, alkali metal alginate and the like, polysaccharides having a hydroxyalkyl or carboxyalkyl such as hydroxypropylcellulose, hydroxypropylmethylcellulose, carboxymethylcellulose sodium and the like, methylcellulose, polyvinylpyrrolidone, polyvinyl alcohol and polyethylene glycol can be mentioned.

The content of a water-insoluble substance in the film agent of a sustained release preparation is from about 30 to about 90% (w/w), preferably from about 35 to about 80% (w/w), further preferably from about 40 to about 75% (w/w), the content of a swellable polymer is from about 3 to about 30% (w/w), preferably from about 3 to about 15% (w/w). The film agent may further contain a hydrophilic substance, and in which case, the content of a hydrophilic substance in the film agent is about 50% (w/w) or less, preferably about 5 to about 40% (w/w), further preferably from about 5 to about 35% (w/w). This % (w/w) indicates % by weight based on a film agent composition which is obtained by removing a solvent (e.g., water, lower alcohols such as methanol, ethanol and the like) from a film agent solution.

The sustained release preparation is produced by preparing a nucleus containing a drugs as exemplified below, then, coating the resulted nucleus with a film agent solution prepared by heat-solving a water-insoluble substance, swellable polymer and the like or by dissolving or dispersing it in a solvent.

### I. Preparation of Nucleus Containing Drug

The form of nucleus containing a drug to be coated with a film agent (hereinafter, sometimes simply referred to as nucleus) is not particularly restricted, and preferably the nucleus is formed into particles such as a granule or fine particle.

When the nucleus is composed of granules or fine particles, the total particle size thereof is preferably from about 150 to about 2000 μm, further preferably from about 500 to about 1400 μm.

Preparation of the nucleus can be effected by a usual production method. For example, a suitable excipient, binding agent, disintegrating agent, lubricant, stabilizer and the like are mixed with a drug, and the mixture is subjected to a wet extrusion granulating method, fluidized bed granulating method or the like, to prepare a nucleus.

The content of drugs in a nucleus is from about 0.5 to about 95% (w/w), preferably from about 5.0 to about 80% (w/w), further preferably from about 30 to about 70% (w/w).

As the excipient contained in the nucleus, for example, saccharides such as sucrose, lactose, mannitol, glucose and the like, starch, crystalline cellulose, calcium phosphate and corn starch are used. Among them, crystalline cellulose, corn starch are preferable.

As the binding agent, for example, polyvinyl alcohol, hydroxypropylcellulose, polyethylene glycol, polyvinyl pyrrolidone, Pluronic F68, gum Arabic, gelatin and starch are used. As the disintegrating agent, for example, carboxymethylcellulose calcium (ECG505), croscarmelose sodium (Ac-Di-Sol), crosslinked polyvinylpyrrolidone (Crospovidone), and low substituted hydroxypropylcellulose (L-HPC) are used. Among them, hydroxypropylcellulose, polyvinylpyrrolidone, lower substituted hydroxypropylcellulose are preferable. As the lubricant and solidification inhibitor, for example, talc, magnesium stearate and inorganic salts thereof are used, and as the lubricant, polyethylene glycol is used. As the stabilizer, acids such as tartaric acid, citric acid, succinic acid, fumaric acid, maleic acid and the like, are used.

A nucleus can also be prepared by, in addition to the above-mentioned productions method, for example, a rolling granulation method in which a drug or a mixture of a drug with an excipient, lubricant and the like is added portionwise onto an inert carrier particle which is the core of the nucleus while spraying a binder dissolved in a suitable solvent such as water, lower alcohol (e.g., methanol, ethanol) and the like, a pan coating method, a fluidized bed coating method or a melt granulating method. As the inert carrier particle, for example, those made of sucrose, lactose, starch, crystalline cellulose or waxes can be used, and the total particle size thereof is preferably from about 100 μm to about 1500 μm.

For separating a drug contained in a nucleus and a film agent, the surface of the nucleus may be coated with a protective agent. As the protective agent, for example, the above-mentioned hydrophilic substances, water-insoluble substances and the like are used. As the protective agent, preferably polyethylene glycol, and polysaccharides having a hydroxyalkyl or carboxyalkyl are used, more preferably hydroxypropylmethylcellulose and hydroxypropylcellulose are used. The protective agent may contain, as stabilizer, acids such as tartaric acid, citric acid, succinic acid, fumaric acid, maleic acid and the like, and lubricants such as talc and the like. When the protective agent is used, the coating amount is from about 1 to about 15% (w/w), preferably from about 1 to about 10% (w/w), further preferably from about 2 to about 8% (w/w), based on the nucleus.

The protective agent can be coated by a usual coating method, and specifically, the protective agent can be coated by spraying the nucleus, for example, by a fluidized bed coating method, pan coating method and the like.

II. Coating of Nucleus with Film Agent

A nucleus obtained in the above-mentioned step I is coated with a film agent solution obtained by heat-solving the above-mentioned water-insoluble substance and pH-dependent swellable polymer, and a hydrophilic substance, or by dissolving or dispersing them in a solvent, to give a sustained release preparation.

As the method for coating a nucleus with a film agent solution, for example, a spray coating method and the like are listed.

The composition ratio of a water-insoluble substance, swellable polymer or hydrophilic substance in a film agent solution is appropriately selected so that the contents of these components in a coated film are the above-mentioned contents, respectively.

The coating amount of a film agent is from about 1 to about 90% (w/w), preferably from about 5 to about 50% (w/w), further preferably from about 5 to about 35% (w/w), based on a nucleus (not including coating amount of protective agent).

As the solvent in a film agent solution, water or an organic solvent can be used alone or in admixture thereof. In the case of use in admixture, the mixing ratio of water to an organic solvent (water/organic solvent: by weight) can be varied in the range from 1 to 100%, and preferably from 1 to about 30%. The organic solvent is not particularly restricted providing it dissolves a water-insoluble substance, and for example, lower alcohols such as methyl alcohol, ethyl alcohol, isopropyl alcohol, n-butyl alcohol and the like, lower alkanone such as acetone and the like, acetonitrile, chloroform, methylene chloride and the like are used. Among them, lower alcohols are preferable, and ethyl alcohol and isopropyl alcohol are particularly preferable. Water, and a mixture of water with an organic solvent are preferably used as a solvent for a film agent. In this case, if necessary, an acid such as tartaric acid, citric acid, succinic acid, fumaric acid, maleic acid and the like may also be added into a film agent solution for stabilizing the film agent solution.

An operation of coating by spray coating can be effected by a usual coating method, and specifically, it can be effected by coating a film agent solution onto a nucleus by a fluidized bed coating method, pan coating method and the like. In this case, if necessary, talc, titanium oxide, magnesium stearate, calcium stearate, light anhydrous silicic acid and the like may also be added as a lubricant, and glycerin fatty acid ester, hydrogenated castor oil, triethyl citrate, cetyl alcohol, stearyl alcohol and the like may also be added as a plasticizer.

After coating with a film agent, if necessary, an antistatic agent such as talc and the like may be mixed.

The immediate-release preparation may be liquid (solution, suspension, emulsion and the like) or solid (particle, pill, tablet and the like). As the immediate-release preparation, oral administration agents and parenteral administration agents such as an injection and the like are used, and oral administration agents are preferable.

The immediate-release preparation, usually, may contain, in addition to an active component drug, also carriers, additives and excipients conventionally used in the pharmaceutical field (hereinafter, sometimes abbreviated as excipient). The excipient used is not particularly restricted providing it is an excipient ordinarily used as a preparation excipient. For example, as the excipient for an oral solid preparation, lactose, starch, corn starch, crystalline cellulose (Avicel PH101, manufactured by Asahi Kasei Corporation, and the like), powder sugar, granulated sugar, mannitol, light anhydrous silicic acid, magnesium carbonate, calcium carbonate, and L-cysteine are listed, and preferably corn starch and mannitol and the like are listed. These excipients can be used alone or in combination of two or more. The content of the excipient is, for example, from about 4.5 to about 99.4 w/w %, preferably from about 20 to about 98.5 w/w %, further preferably from about 30 to about 97 w/w %, based on the total amount of the immediate-release preparation.

The content of a drug in the immediate-release preparation can be appropriately selected in the range from about 0.5 to about 95 w/w %, preferably from about 1 to about 60 w/w % based on the total amount of the immediate-release preparation.

When the immediate-release preparation is an oral solid preparation, it usually contains, in addition to the above-mentioned components, also a disintegrating agent. As this disintegrating agent, for example, carboxymethylcellulose calcium (ECG-505, manufactured by Gotoku Yakuhin), croscarmelose sodium (e.g., Actisol, manufactured by Asahi Kasei Corporation), crospovidone (e.g., Kollidon CL, manufactured by BASF), low substituted hydroxypropylcellulose (manufactured by Shin-Etsu Chemical Co., Ltd.), carboxymethylstarch (manufactured by Matsutani Kagaku K.K.), carboxymethylstarch sodium (Exprotab, manufactured by Kimura Sangyo), partially pregelatinized starch (PCS, manufactured by Asahi Kasei Corporation), and the like are used, and for example, those which disintegrate a granule by absorbing water in contact with water, causing swelling, or making a channel between an effective ingredient constituting the nucleus and an excipient, can be used. These disintegrating agents can be used alone or in combination of two or more. The amount of the disintegrating agent used is appropriately selected depending on the kind and blending amount of a drug used, design of releasing property, and the like, and for example, from about 0.05 to about 30 w/w %, preferably from about 0.5 to about 15 w/w %, based on the total amount of the immediate-release preparation.

When the immediate-release preparation is an oral solid preparation, it may further contain, in addition to the above-mentioned composition, if desired, additives conventional in solid preparations. As such an additive, there are used, for example, a binder (e.g., sucrose, gelatin, gum Arabic powder, methylcellulose, hydroxypropylcellulose, hydroxypropylmethylcellulose, carboxymethylcellulose, polyvinylpyrrolidone, pullulan, dextrin and the like), a lubricant (e.g., polyethylene glycol, magnesium stearate, talc, light anhydrous silicic acid (e.g., Aerosil (manufactured by Nippon Aerosil)), a surfactant (e.g., anionic surfactants such as sodium alkylsulfate and the like, nonionic surfactants such as polyoxyethylene fatty acid ester and polyoxyethylene sorbitan ester of fatty acid, polyoxyethylene castor oil derivatives and the like), a colorant (e.g., tar coloring matter, caramel, iron oxide red, titanium oxide, riboflavins), if necessary, an appetizing agent (e.g., sweetening agent, flavoring agent and the like), an adsorbent, preservative, wetting agent, antistatic agent, and the like. Further, as the stabilizer, an organic acid such as tartaric acid, citric acid, succinic acid, fumaric acid and the like may also be added.

As the above-mentioned binder, hydroxypropylcellulose, polyethylene glycol and polyvinylpyrrolidone and the like are preferably used.

The immediate-release preparation can be prepared by, based on a usual technology of producing preparations, mixing the above-mentioned components, and if necessary, further kneading the mixture, and molding it. The above-mentioned mixing is conducted by generally used methods, for example, mixing, kneading and the like. Specifically, when a immediate-release preparation is formed, for example, into a particle, it can be prepared, according to the same means as in the above-mentioned method for preparing a nucleus of a sustained release preparation, by mixing the components using a vertical granulator, universal kneader (manufactured by Hata Tekkosho), fluidized bed granulator FD-5S (manufactured by Powrex Corporation), and the like, and then, granulating the mixture by a wet extrusion granulation method, fluidized bed granulation method and the like.

Thus obtained immediate-release preparation and sustained release preparation may be themselves made into products or made into products appropriately together with preparation excipients and the like, separately, by an ordinary method, then, may be administered simultaneously or may be administered in combination at any administration interval, or they may be themselves made into one preparation for oral administration (e.g., granule, fine particle, tablet, capsule and the like) or made into one preparation for oral administration appropriately together with preparation excipients and the like. It may also be permissible that they are made into granules or fine particles, and filled in the same capsule to be used as a preparation for oral administration.

### [3] Sublingual, Buccal or Intraoral Quick Disintegrating Agent and Preparation Thereof.

Sublingual, buccal or intraoral quick disintegrating agents may be a solid preparation such as tablet and the like, or may be an oral mucosa membrane patch (film).

As the sublingual, buccal or intraoral quick disintegrating agent, a preparation containing the compound of the present invention or the concomitant drug and an excipient is preferable. It may contain also auxiliary agents such as a lubricant, isotonizing agent, hydrophilic carrier, water-dispersible polymer, stabilizer and the like. Further, for easy absorption and increased in vivo use efficiency, β-cyclodextrin or β-cyclodextrin derivatives (e.g., hydroxypropyl-β-cyclodextrin and the like) and the like may also be contained.

As the above-mentioned excipient, lactose, sucrose, D-mannitol, starch, crystalline cellulose, light anhydrous silicic acid and the like are listed. As the lubricant, magnesium stearate, calcium stearate, talc, colloidal silica and the like are listed, and particularly, magnesium stearate and colloidal silica are preferable. As the isotonizing agent, sodium chloride, glucose, fructose, mannitol, sorbitol, lactose, saccharose, glycerin, urea and the like are listed, and particularly, mannitol is preferable. As the hydrophilic carrier, swellable hydrophilic carriers such as crystalline cellulose, ethylcellulose, crosslinkable polyvinylpyrrolidone, light anhydrous silicic acid, silicic acid, dicalcium phosphate, calcium carbonate and the like are listed, and particularly, crystalline cellulose (e.g., crystalline cellulose and the like) is preferable. As the water-dispersible polymer, gums (e.g., gum tragacanth, acacia gum, cyamoposis gum), alginates (e.g., sodium alginate), cellulose derivatives (e.g., methylcellulose, carboxymethylcellulose, hydroxymethylcellulose, hydroxypropylcellulose, hydroxypropylmethylcellulose), gelatin, water-soluble starch, polyacrylic acids (e.g., Carbomer), polymethacrylic acid, polyvinyl alcohol, polyethylene glycol, polyvinylpyrrolidone, polycarbophil, ascorbic acid, palmitates and the like are listed, and hydroxypropylmethylcellulose, polyacrylic acid, alginate, gelatin, carboxymethylcellulose, polyvinylpyrrolidone, polyethylene glycol and the like are preferable. Particularly, hydroxypropylmethylcellulose is preferable. As the stabilizer, cysteine, thiosorbitol, tartaric acid, citric acid, sodium carbonate, ascorbic acid, glycine, sodium sulfite and the like are listed, and particularly, citric acid and ascorbic acid are preferable.

The sublingual, buccal or intraoral quick disintegrating agent can be produced by mixing the compound of the present invention or the concomitant drug and an excipient by a method known per se. Further, if desired, the above-mentioned auxiliary agents such as a lubricant, isotonizing agent, hydrophilic carrier, water-dispersible polymer, stabilizer, colorant, sweetening agent, preservative and the like may be mixed. The sublingual, buccal or intraoral quick disintegrating agent is obtained by mixing the above-mentioned components simultaneously or at a time interval, then subjecting the mixture to tablet-making molding under pressure. For obtaining suitable hardness, it may also be permissible that the materials are moistened by using a solvent such as water, alcohol and the like if desired before and after the tablet making process, and after the molding, the materials are dried, to obtain a product.

In the case of molding into a mucosa membrane patch (film), the compound of the present invention or the concomitant drug and the above-mentioned water-dispersible polymer (preferably hydroxypropylcellulose hydroxypropylmethylcellulose), excipient and the like are dissolved in a solvent such as water and the like, and the resulted solution is cast to give a film. Further, additives such as a plasticizer, stabilizer, antioxidant, preservative, colorant, buffer, sweetening agent and the like may also be added. For imparting suitable elasticity to the film, glycols such as polyethylene glycol, propylene glycol and the like may be contained, or for enhancing adhesion of the film to an intraoral mucosa membrane lining, a bio-adhesive polymer (e.g., polycarbophil, carbopol) may also be contained. In the casting, a solution is poured on the non-adhesive surface, spread to uniform thickness (preferably about 10 to 1000 micron) by an application tool such as a doctor blade and the like, then, the solution is dried to form a film. It may be advantageous that thus formed film is dried at room temperature or under heat, and cut into a desired area.

A preferable intraorally quick-integrating agent is a solid quick-diffusable administration agent comprised of a network structure consisting of the compound of the present invention or a concomitant drug, and a water-soluble or water-diffusible carrier inactive to the compound of the present invention or the concomitant drug. The network structure can be obtained by evaporating a solvent from a composition comprised of a solution of the compound of the present invention or a concomitant drug in a suitable solvent.

It is preferable that the composition of an intraoral quick disintegrating agent contains a matrix forming agent and a secondary component, in addition to the compound of the present invention or the concomitant drug.

Examples of the matrix forming agent include gelatins, dextrins, animal proteins or vegetable proteins such as soybean, wheat and psyllium seed protein and the like; rubber substances such as gum Arabic, guar gum, agar, xanthan and the like; polysaccharides; alginic acids; carboxymethylcelluloses; carageenans; dextrans; pectines; synthetic polymers such as polyvinylpyrrolidone and the like; substances derived from a gelatin-gum Arabic complex, and the like. Further, saccharides such as mannitol, dextrose, lactose, galactose, trehalose and the like; cyclic saccharides such as cyclodextrin and the like; inorganic salts such as sodium phosphate, sodium chloride and aluminum silicate and the like; amino acids having 2 to 12 carbon atoms such as glycine, L-alanine, L-aspartic acid, L-glutamic acid, L-hydroxyproline, L-isoleucine, L-leucine, L-phenylalanine and the like, are contained.

One or more of the matrix forming agents can be introduced in a solution or suspension before solidification. Such as matrix forming agent may be present in addition to a surfactant, or may be present while a surfactant being excluded. The matrix forming agents aid to maintain the compound of the present invention or the concomitant drug in the solution or suspension in diffused condition, in addition to formation of the matrix.

The composition may contain secondary components such as a preservative, antioxidant, surfactant, thickening agent, colorant, pH controlling agent, flavoring agent, sweetening agent, food taste masking agent and the like. As the suitable colorant, there are listed red, black and yellow iron oxides, and FD & C dyes such as FD & C Blue 2, FD & C Red 40 and the like manufactured by Ellis and Everard. Examples of the suitable flavoring agent include mint, raspberry, licorice, orange, lemon, grapefruit, caramel, vanilla, cherry, grape flavor and combinations thereof. Examples of the suitable pH controlling agent include citric acid, tartaric acid, phosphoric acid, hydrochloric acid and maleic acid. Examples of the suitable sweetening agent include aspartame, acesulfame K and thaumatin and the like. Examples of the suitable food taste masking agent include sodium bicarbonate, ion exchange resin, cyclodextrin-inclusion compounds, adsorbent substances and microcapsulated apomorphine.

The preparation contains the compound of the present invention or the concomitant drug in an amount usually from about 0.1 to about 50% by weight, preferably from about 0.1 to about 30% by weight, and preferable are preparations (such as the above-mentioned sublingual agent, buccal and the like) which can dissolve 90% or more of the compound of the present invention or the concomitant drug (into water) within the time range of about 1 to about 60 min, preferably of about 1 to about 15 min, more preferably of about 2 to about 5 min, and intraoral quick disintegrating preparations which are disintegrated within the range of 1 to 60 sec, preferably of 1 to 30 sec, further preferably of 1 to 10 sec, after placed in an oral cavity.

The content of the above-mentioned excipient in the whole preparation is from about 10 to about 99% by weight, preferably from about 30 to about 90% by weight. The content of 3-cyclodextrin or β-cyclodextrin derivative in the whole preparation is from 0 to about 30% by weight. The content of the lubricant in the whole preparation is from about 0.01 to about 10% by weight, preferably from about 1 to about 5% by weight. The content of the isotonizing agent in the whole preparation is from about 0.1 to about 90% by weight, preferably from about 10 to about 70% by weight. The content of the hydrophilic carrier in the whole preparation is from about 0.1 to about 50% by weight, preferably from about 10 to about 30% by weight. The content of the water-dispersible polymer in the whole preparation is from about 0.1 to about 30% by weight, preferably from about 10 to about 25% by weight. The content of the stabilizer in the whole preparation is from about 0.1 to about 10% by weight, preferably from about 1 to 5% by weight. The above-mentioned preparation may further contain additives such as a colorant, sweetening agent, preservative and the like, if necessary.

The dosage of a combination agent of the present invention differs depending on the kind of a compound of the present invention, age, body weight, condition, drug form, administration method, administration period and the like, and for example, for one cancer patient (adult, body weight: about 60 kg), the combination agent is administered intravenously, at a dose of about 0.01 to about 1000 mg/kg/day, preferably about 0.01 to about 100 mg/kg/day, more preferably about 0.1 to about 100 mg/kg/day, particularly about 0.1 to about 50 mg/kg/day, especially about 1.5 to about 30 mg/kg/day, in terms of the compound of the present invention or the concomitant drug, respectively, once or several times in division a day. Of course, since the dose as described above varies depending on various conditions, amounts smaller than the above-mentioned dosage may sometimes be sufficient, further, amounts over that range sometimes have to be administered.

The amount of the concomitant drug can be set at any value unless side effects are problematical. The daily dosage in terms of the concomitant drug differs depending on the severity of the symptom, age, sex, body weight, sensitivity difference of the administration subject, administration period, interval, and nature, pharmacy, kind of the pharmaceutical preparation, kind of effective ingredient, and the like, and not particularly restricted, and the amount of a drug is, in the case of oral administration for example, usually from about 0.001 to 2000 mg, preferably from about 0.01 to 500 mg, further preferably from about 0.1 to 100 mg, per 1 kg of a mammal, which is usually administered once to 4-times in division a day.

In administration of a combination agent of the present invention, the compound of the present invention may be administered after administration of the concomitant drug or the concomitant drug may be administered after administration of the compound of the present invention, though they may be administered simultaneously. When administered at a time interval, the interval differs depending on the effective ingredient to be administered, drug form and administration method, and for example, when the concomitant drug is administered first, a method in which the compound of the present invention is administered within time range of from 1 min to 3 days, preferably from 10 min to 1 day, more preferably from 15 min to 1 hr after administration of the concomitant drug is exemplified. When the compound of the present invention is administered first, a method in which the concomitant drug is administered within time range of from 1 min to 1 day, preferably from 10 min to 6 hrs, more preferably from 15 min to 1 hr after administration of the compound of the present invention is exemplified.

In a preferable administration method, for example, the concomitant drug which has been molded into an oral administration preparation is administered orally at a daily dose of about 0.001 to 200 mg/kg, and about 15 min later, the compound of the present invention which has been molded into an oral administration preparation is administered orally at a daily dose of about 0.005 to 100 mg/kg.

Furthermore, the compound of the present invention or the combination agent of the present invention can be used concurrently with a non-drug therapy. To be precise, the compound of the present invention or the combination agent of the present invention can be combined with a non-drug therapy such as (1) surgery, (2) hypertensive chemotherapy using angiotensin II etc., (3) gene therapy, (4) thermotherapy, (5) cryotherapy, (6) laser cauterization, (7) radiotherapy, and the like.

For example, by using the compound of the present invention or the combination agent of the present invention before or after an surgery and the like, or before or after a combined treatment of two or three kinds thereof, effects such as prevention of emergence of resistance, prolongation of Disease-Free Survival, suppression of cancer metastasis or recurrence, prolongation of life and the like can be afforded.

In addition, it is possible to combine a treatment with the compound of the present invention or the combination agent of the present invention with a supportive therapy [(i) administration of antibiotic (e.g., β-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a medicament for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a medicament for suppressing multiple drug resistance of cancer and the like].

Preferably, the compound of the present invention or the combination agent of the present invention is administered orally (including sustained-release preparations), intravenously (including boluses, infusions and clathrates), subcutaneously and intramuscularly (including boluses, infusions and sustained-release preparations), transdermally, intratumorally or proximally before or after the above-described treatment is conducted.

As a period for administering the compound of the present invention or the combination agent of the present invention before the surgery, etc., for example, it can be administrated 1-time about 30 min to 24 hrs before the surgery, etc., or in 1 to 3 cycles about 3 months to 6 months before the surgery, etc. In this way, the surgery, etc. can be conducted easily because, for example, a cancer tissue would be reduced by administering the compound of the present invention or the combination agent of the present invention before the surgery, and the like.

As a period for administering the compound of the present invention or the combination agent of the present invention after the surgery, etc., for example, it can be administrated repeatedly per a few weeks to 3 months, about 30 min to 24 hrs after the surgery, and the like. In this way, it enhances the effect of the surgery, etc. by administering the compound of the present invention or the combination agent of the present invention after the surgery, and the like.

### EXAMPLES

The present invention is explained in more detail in the following by referring to Examples, Formulation Examples and

Experimental Examples. However, the present invention is not limited to the Examples.

### Example 1

### Production of (3S,8aR)—N-benzyl-2-{(2S)-2-cyclohexyl-2-[(N-methyl-L-alanyl)amino]acetyl}octahydropyrrolo[1,2-a]pyrazine-3-carboxamide dihydrochloride

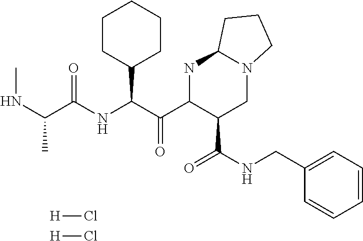

### (i) Production of N-benzyl-5-oxo-D-prolinamide

5-oxo-D-Proline (5.0 g), benzylamine (4.65 mL), 1-ethyl-3-(3-dimethylaminopropyl)carbodiimide hydrochloride (8.5 g) and 1-hydroxybenzotriazole (6.3 g) were mixed in acetonitrile (100 mL) under ice-cooling, and the mixture was allowed to warm to room temperature and stirred for 3 hr. The mixture was diluted with ethyl acetate (200 mL), and washed with 1N hydrochloric acid (50 mL), saturated aqueous sodium hydrogen carbonate solution. (50 mL) and saturated brine (50 mL). The organic layer was dried over anhydrous magnesium sulfate, and concentrated under reduced pressure. The precipitated solid was collected by filtration, washed with diethyl ether, and dried under reduced pressure to give the title compound (4.02 g) as a white powder.

¹H NMR (DMSO-d₆, 300 MHz): δ 1.82-1.96 (1H, m), 2.02-2.36 (3H, m), 3.99-4.09 (1H, m), 4.29 (2H, d, J=5.9 Hz), 7.18-7.38 (5H, m), 7.85 (1H, s), 8.50 (1H, t, J=5.9 Hz).

### (ii) Production of 1-phenyl-N-[(2R)-pyrrolidin-2-ylmethyl]methanamine

To a suspension of lithium aluminum hydride (5.40 g) in tetrahydrofuran (150 mL) was added dropwise a suspension (350 mL) of N-benzyl-5-oxo-D-prolinamide (11.0 g) in tetrahydrofuran under ice-cooling, and the mixture was stirred with heating at 60° C. for 14 hr. The reaction mixture was cooled to 0° C., water (10.8 mL), 1N aqueous sodium hydroxide solution (5.4 mL) and water (5.4 mL) were successively added thereto, and the resulting insoluble material was filtered off. The filtrate was concentrated under reduced pressure to give the title compound (8.95 g) as a pale-yellow oil. Without further purification, the compound was used for the next reaction.

¹H NMR (DMSO-d₆, 300 MHz): δ 1.18-1.33 (1H, m), 1.48-1.81 (3H, m), 2.09 (2H, m), 2.30-2.44 (2H, m), 2.63-2.80 (2H, m), 3.00-3.12 (1H, m), 3.68 (2H, s), 7.13-7.37 (5H, m).

### (iii) Production of methyl(3S,8aR)-2-benzyloctahydropyrrolo[1,2-a]pyrazine-3-carboxylate

To a suspension of 1-phenyl-N-[(2R)-pyrrolidin-2-ylmethyl]methanamine (13.6 g) in toluene (120 mL) were added triethylamine (22.9 mL) and methyl 2,3-dibromopropanoate (13.4 g) under ice-cooling, and the mixture was stirred with heating at 90° C. for 5 hr. The reaction mixture was allowed to cool to room temperature, and diluted with diethyl ether (200 mL) and saturated brine (200 mL). The organic layer was dried over anhydrous magnesium sulfate, and the insoluble material was filtered off. The filtrate was concentrated under reduced pressure, and the residue was purified by silica gel column chromatography (ethyl acetate/hexane=10/80→50/50) to give the title compound (6.86 g) as a pale-yellow oil.

¹H NMR (DMSO-d₆, 300 MHz): δ 1.14-1.28 (1H, m), 1.53-1.77 (3H, m), 1.83-2.00 (2H, m), 2.31 (1H, dd, J=10.7, 3.9 Hz), 2.61-2.95 (3H, m), 3.29 (1H, dd, J=10.7, 2.0 Hz), 3.53 (1H, dd, J=3.7, 1.8 Hz), 3.62 (3H, s), 3.89 (2H, s), 7.17-7.37 (5H, m).

### (iv) Production of methyl(3S,8aR)-octahydropyrrolo[1,2-a]pyrazine-3-carboxylate dihydrochloride

Methyl(3S,8aR)-2-benzyloctahydropyrrolo[1,2-a]pyrazine-3-carboxylate (400 mg) was dissolved in ethanol (3 mL), 10% palladium-carbon (60 mg, 50% wet) and 4N hydrogen chloride-ethyl acetate solution (1 mL) were added thereto, and the mixture was stirred at room temperature for 4 hr under a hydrogen atmosphere (1 atm). The insoluble material was filtered off through a celite pad, and the filtrate was concentrated to give the title compound (370 mg) as a pale-yellow oil. LC-MS: 185 (MH⁺).

### (v) Production of methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate

Methyl(3S,8aR)-octahydropyrrolo[1,2-a]pyrazine-3-carboxylate dihydrochloride (370 mg), (2S)-{[N-(tert-butoxycarbonyl)-N-methyl-L-alanyl]amino}(cyclohexyl)ethanoic acid (540 mg), 4-(4,6-dimethoxy-1,3,5-triazin-2-yl)-4-methylmorpholinium chloride (475 mg) and 4-methylmorpholine (0.314 mL) were mixed in tetrahydrofuran under ice-cooling, and the mixture was allowed to warm to room temperature, and stirred for 4 hr. The mixture was diluted with ethyl acetate (50 mL), and washed with water (50 mL) and saturated brine (50 mL). The organic layer was dried over anhydrous magnesium sulfate, and concentrated under reduced pressure. The residue was purified by silica gel column chromatography (ethyl acetate/hexane=10/80-100/0) to give the title compound (320 mg) as a pale-yellow oil. LC-MS: 509 (MH⁺).

### (vi) Production of (3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylic acid

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (320 mg) was dissolved in tetrahydrofuran (3 mL), a solution prepared by dissolving lithium hydroxide monohydrate (34 mg) in water (3 mL) was added thereto, and the mixture was stirred at room temperature for 3 hr. To the mixture was added 1N hydrochloric acid (0.818 mL), and the mixture was concentrated under reduced pressure to give the title compound (300 mg) as a crude product. Without further purification, the compound was used for the next reaction. LC-MS: 495 (MH⁺).

### (vii) Production of (3S,8aR)—N-benzyl-2-{(2S)-2-cyclohexyl-2-[(N-methyl-L-alanyl)amino]acetyl}octahydropyrrolo[1,2-a]pyrazine-3-carboxamide dihydrochloride

(3S,8aR)-2-[(2S)-2-{[N-(tert-Butoxycarbonyl)-N-methyl-L-alanyl]amino}-2-cyclohexylacetyl]octahydropyrrolo[1,2-a]pyrazine-3-carboxylic acid (150 mg), benzylamine (0.040 mL), 4-(4,6-dimethoxy-1,3,5-triazin-2-yl)-4-methylmorpholinium chloride (134 mg) and 4-methylmorpholine (0.033 mL) were mixed in tetrahydrofuran (5 mL) under ice-cooling, and the mixture was allowed to warm to room temperature, and stirred for 5 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/80→100/0→ethyl acetate/methanol=90/10) to give a colorless amorphous powder. This amorphous powder was dissolved in ethyl acetate (1 mL) and methanol (1 mL), 4M hydrogen chloride-ethyl acetate solution (1 mL) was added thereto, and the mixture was stirred at room temperature for 2 hr. The mixture was concentrated under reduced pressure, and the residue was collected by filtration, washed with ether, and dried under reduced pressure to give the title compound (40 mg) as a colorless amorphous powder.

¹H NMR (DMSO-d₆, 300 MHz): δ 0.77-1.26 (7H, m), 1.28-1.44 (3H, m), 1.48-1.83 (8H, m), 1.89-2.19 (2H, m), 3.05 (1H, brs), 3.44-3.96 (6H, m), 3.98-4.85 (5H, m), 7.11-7.43 (5H, m), 8.69-9.01 (3H, m), 9.35 (1H, brs), 12.02 (1H, brs).

### Example 2

### Production of (3S,8aR)-2-{(2S)-2-cyclohexyl-2-[(N-methyl-L-alanyl)amino]acetyl}-N-[(1R)-1,2,3,4-tetrahydronaphthalen-1-yl]octahydropyrrolo[1,2-a]pyrazine-3-carboxamide dihydrochloride

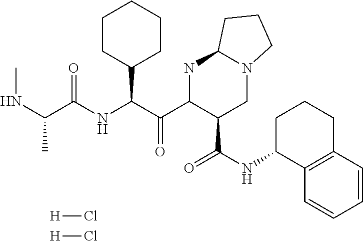

(3S,8aR)-2-[(2S)-2-{[N-(tert-Butoxycarbonyl)-N-methyl-L-alanyl]amino}-2-cyclohexylacetyl]octahydropyrrolo[1,2-a]pyrazine-3-carboxylic acid (190 mg) synthesized in the above-mentioned Example 1 (vi), (1R)-1,2,3,4-tetrahydronaphthalen-1-amine (68 mg), 4-(4,6-dimethoxy-1,3,5-triazin-2-yl)-4-methylmorpholinium chloride (170 mg) and 4-methylmorpholine (0.043 mL) were mixed in tetrahydrofuran (5 mL) under ice-cooling, and the mixture was allowed to warm to room temperature, and stirred for 5 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/80→100/0, and then ethyl acetate/methanol=90/10) to give a colorless amorphous powder. This amorphous powder was dissolved in ethyl acetate (1 mL), 4M hydrogen chloride-ethyl acetate solution (1 mL) was added thereto, and the mixture was stirred at room temperature for 2 hr. The mixture was concentrated under reduced pressure, and the residue was collected by filtration, washed with ether, and dried under reduced pressure to give the title compound (120 mg) as a colorless amorphous powder.

¹H NMR (DMSO-d₆, 300 MHz): δ 0.96-1.27 (5H, m), 1.30-1.44 (3H, m), 1.49-1.92 (12H, m), 1.97-2.20 (2H, m), 2.40-2.47 (3H, m), 2.62-2.85 (2H, m), 3.05 (1H, brs), 3.56 (2H, brs), 3.71 (2H, brs), 3.87 (1H, brs), 4.32-5.07 (4H, m), 7.00-7.29 (4H, m), 8.79 (2H, dd, J=17.5, 8.4 Hz), 8.97 (1H, brs), 9.40 (1H, brs), 11.92-12.56 (1H, m).

### Example 3

### Production of (3S,8aR)-2-{(2S)-2-cyclohexyl-2-[(N-methyl-L-alanyl)amino]acetyl}-N-(diphenylmethyl)octahydropyrrolo[1,2-a]pyrazine-3-carboxamide dihydrochloride

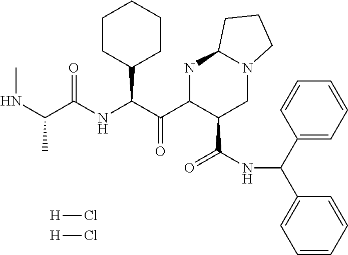

(3S,8aR)-2-[(2S)-2-{[N-(tert-Butoxycarbonyl)-N-methyl-L-alanyl]amino}-2-cyclohexylacetyl]octahydropyrrolo[1,2-a]pyrazine-3-carboxylic acid (190 mg) synthesized in the above-mentioned Example 1 (vi), 1,1-diphenylmethanamine (85 mg), 4-(4,6-dimethoxy-1,3,5-triazin-2-yl)-4-methylmorpholinium chloride (170 mg) and 4-methylmorpholine (0.043 mL) were mixed in tetrahydrofuran (5 mL) under ice-cooling, and the mixture was allowed to warm to room temperature, and stirred for 5 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/80→100/0→ethyl acetate/methanol=90/10) to give a colorless amorphous powder. This amorphous powder was dissolved in ethyl acetate (1 mL), 4M hydrogen chloride-ethyl acetate solution (1 mL) was added thereto, and the mixture was stirred at room temperature for 2 hr. The mixture was concentrated under reduced pressure, and the residue was collected by filtration, washed with ether, and dried under reduced pressure to give the title compound (135 mg) as a colorless amorphous powder.

¹H NMR (DMSO-d₆, 300 MHz): δ 0.87-1.23 (5H, m), 1.37 (3H, d, J=6.8 Hz), 1.73 (8H, d, J=10.0 Hz), 1.93-2.17 (2H, m), 2.40-2.48 (3H, m), 3.02 (1H, brs), 3.47-3.93 (5H, m), 4.45 (1H, brs), 4.67-4.87 (2H, m), 6.07 (1H, d, J=8.1 Hz), 7.13-7.48 (10H, m), 8.79 (2H, d, J=8.1 Hz), 9.34 (2H, d, J=8.3 Hz), 12.01 (1H, brs).

### Example 4

### Production of N-{(1S)-1-cyclohexyl-2-[(3S,8aR)-3-(2,3-dihydro-1H-indol-1-ylmethyl)hexahydropyrrolo[1,2-a]pyrazin-2(1H)-yl]-2-oxoethyl}-N-2-methyl-L-alaninamide trihydrochloride

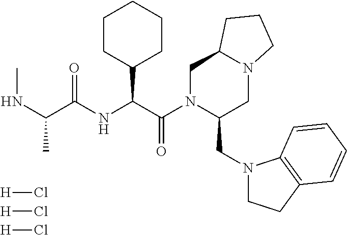

### (i) Production of 2-tert-butyl 3-methyl(3S,8aR)-hexahydropyrrolo[1,2-a]pyrazine-2,3(1H)-dicarboxylate

To a solution of methyl(3S,8aR)-2-benzyloctahydropyrrolo[1,2-a]pyrazine-3-carboxylate (6.80 g) synthesized in the above-mentioned Example 1 (iii) in 5-10% hydrogen chloride-methanol was added 10% palladium-carbon (680 mg, 50% wet), and the mixture was stirred at room temperature for 10 hr under a hydrogen atmosphere (1 atm). The insoluble material was filtered off through a celite pad, and the filtrate was concentrated to give a pale-yellow oil. The obtained methyl(3S,8aR)-octahydropyrrolo[1,2-a]pyrazine-3-carboxylate dihydrochloride was dissolved in saturated aqueous sodium hydrogen carbonate solution (25 mL) and tetrahydrofuran (50 mL), di-tert-butyl bicarbonate (5.68 g) was added thereto, and the mixture was stirred at room temperature for 1 hr. The mixture was diluted with ethyl acetate (300 mL), and washed with water (100 mL) and saturated brine (100 mL). The organic layer was dried over anhydrous magnesium sulfate, and concentrated under reduced pressure. The residue was purified by silica gel column chromatography (ethyl acetate/hexane=10/90→20/80) to give the title compound (6.50 g) as a colorless oil.

¹H NMR (300 MHz, CDCl₃): δ 1.18-1.39 (1H, m), 1.42-1.51 (9H, m), 1.61-1.94 (4H, m), 1.97-2.12 (1H, m), 1.99-2.11 (1H, m), 2.22-2.33 (1H, m), 2.69-2.91 (1H, m), 2.99-3.09 (1H, m), 3.48-3.58 (1H, m), 3.72-3.78 (2H, m), 3.93-4.13 (1H, m), 4.56-4.82 (1H, m).

### (ii) Production of tert-butyl(3S,8aR)-3-(2,3-dihydro-1H-indol-1-ylcarbonyl)hexahydropyrrolo[1,2-a]pyrazine-2(1H)-carboxylate

2-tert-Butyl 3-methyl(3S,8aR)-hexahydropyrrolo[1,2-a]pyrazine-2,3(1H)-dicarboxylate (500 mg) was dissolved in tetrahydrofuran (5 mL), a solution prepared by dissolving lithium hydroxide monohydrate (96 mg) in water (5 mL) was added thereto, and the mixture was stirred at 50° C. for 3 hr. To the mixture was added 1N hydrochloric acid (2.29 mL), and the mixture was concentrated under reduced pressure to give a colorless amorphous powder. This amorphous powder, indoline (210 mg), 1-ethyl-3-(3-dimethylaminopropyl)carbodiimide hydrochloride (510 mg) and 1-hydroxybenzotriazole (360 mg) were mixed in N,N-dimethylformamide, and the mixture was stirred at room temperature for 5 hr. The mixture was diluted with ethyl acetate (50 mL), and washed with water (50 mL) and saturated brine (50 mL). The organic layer was dried over anhydrous magnesium sulfate, and concentrated under reduced pressure. The residue was purified by silica gel column chromatography (ethyl acetate/hexane=10/80→50/50) to give the title compound (150 mg) as a colorless amorphous powder.

¹H NMR (DMSO-d₆, 300 MHz): δ 1.22-1.45 (10H, m), 1.66 (2H, brs), 1.76-1.89 (2H, m), 1.90-2.03 (1H, m), 2.38 (1H, td, J=11.3, 5.4 Hz), 2.85-3.00 (1H, m), 3.17 (3H, t, J=9.1 Hz), 3.36-3.45 (1H, m), 3.81-3.94 (1H, m), 3.96-4.18 (2H, m), 4.70 (1H, brs), 6.94-7.08 (1H, m), 7.09-7.20 (1H, m), 7.25

(1H, d, J=7.0 Hz), 8.02 (1H, brs).

### (iii) Production of (3S,8aR)-3-(2,3-dihydro-1H-indol-1-ylmethyl)octahydropyrrolo[1,2-a]pyrazine

tert-Butyl(3S,8aR)-3-(2,3-dihydro-1H-indol-1-ylcarbonyl)hexahydropyrrolo[1,2-a]pyrazine-2(1H)-carboxylate (150 mg) was dissolved in ethyl acetate (5 mL), 4M hydrogen chloride-ethyl acetate solution (5 mL) was added thereto, and the mixture was stirred at room temperature for 1 hr. The mixture was concentrated under reduced pressure to give a colorless amorphous powder. The amorphous powder was dissolved in tetrahydrofuran (5 mL), and the solution was added dropwise to a suspension of lithium aluminum hydride (77 mg) in tetrahydrofuran (5 mL) under ice-cooling, and the mixture was stirred with heating under reflux for 5 hr. The reaction mixture was cooled to 0° C., sodium sulfate decahydrate was added until the generation of gas ceased, and the insoluble material was filtered off. The filtrate was concentrated to give the title compound (95 mg) as a pale-yellow oil. LC-MS: 258 (MH⁺)

### (iv) Production of N-{(1S)-1-cyclohexyl-2-[(3S,8aR)-3-(2,3-dihydro-1H-indol-1-ylmethyl)hexahydropyrrolo[1,2-a]pyrazin-2(1H)-yl]-2-oxoethyl}-N-2-methyl-L-alaninamide trihydrochloride

(3S,8aR)-3-(2,3-Dihydro-1H-indol-1-ylmethyl)octahydropyrrolo[1,2-a]pyrazine (98 mg), (2S)-{[N-(tert-butoxycarbonyl)-N-methyl-L-alanyl]amino}(cyclohexyl)ethanoic acid (95 mg) and 4-(4,6-dimethoxy-1,3,5-triazin-2-yl)-4-methylmorpholinium chloride (115 mg) were mixed in tetrahydrofuran (5 mL) under ice-cooling, and the mixture was allowed to warm to room temperature, and stirred for 4 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/80→100/0) to give a colorless amorphous powder. To this amorphous powder were added ethyl acetate (1 mL) and 4M hydrogen chloride-ethyl acetate solution (1 mL), and the mixture was stirred at room temperature for 2 hr. The mixture was concentrated under reduced pressure, and the residue was collected by filtration, washed with ether, and dried under reduced pressure to give the title compound (35 mg) as a colorless amorphous powder.

¹H NMR (DMSO-d₆, 300 MHz): δ 0.57-1.25 (6H, m), 1.38 (5H, dd, J=17.8, 6.8 Hz), 1.60 (4H, brs), 1.89-2.31 (4H, m), 2.38-2.47 (3H, m), 2.62-3.21 (6H, m), 3.23-3.76 (6H, m), 3.81-3.96 (1H, m), 4.32-5.26 (3H, m), 6.42-6.77 (1H, m), 6.84-7.12 (2H, m), 8.57-9.00 (2H, m), 9.32 (1H, brs), 11.10-11.65 (2H, m).

### Example 5

### Production of benzyl{(4S)-4-[(N-methyl-L-alanyl)amino]-5-oxo-5-[(3S,8aR)-3-((1R)-1,2,3,4-tetrahydronaphthalen-1-ylcarbamoyl)hexahydropyrrolo[1,2-a]pyrazin-2(1H)-yl]pentyl}carbamate dihydrochloride

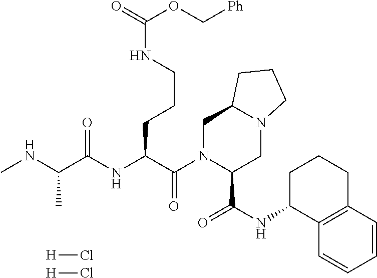

### (i) Production of N-(tert-butoxycarbonyl)-N-methyl-L-alanyl-N-5-[(benzyloxy)carbonyl]-L-ornithine

N-(tert-Butoxycarbonyl)-N-methyl-L-alanine (2.5 g), methyl N-5-[(benzyloxy)carbonyl]-L-ornithinate hydrochloride (3.90 g), 2-chloro-4,6-dimethoxy-1,3,5-triazine (2.27 g) and 4-methylmorpholine (3.38 mL) were mixed in ethyl acetate (100 mL) under ice-cooling, and the mixture was allowed to warm to room temperature, and stirred for 5 hr. The mixture was washed with water (50 mL) and saturated brine (50 mL). The organic layer was dried over anhydrous magnesium sulfate, and concentrated under reduced pressure. The residue was subjected to silica gel column chromatography (ethyl acetate/hexane=10/90→40/60), and the collected fractions were concentrated to give a colorless amorphous powder (5.58 g). This amorphous powder was dissolved in tetrahydrofuran (40 mL), and an aqueous solution (10 mL) of lithium hydroxide monohydrate (671 mg) was added thereto under ice-cooling. The reaction mixture was stirred at room temperature for 2 hr, 10% aqueous citric acid solution was added thereto. The mixture was extracted with ethyl acetate (300 mL), and the organic layer was washed with saturated aqueous sodium hydrogen carbonate solution (50 mL) and saturated brine (50 mL), and dried over anhydrous magnesium sulfate. The insoluble material was filtered off, and the filtrate was concentrated, and dried under reduced pressure to give the title compound (5.00 g) as a colorless amorphous powder.

¹H NMR (300 MHz, DMSO-d₆): δ 1.24 (3H, d, J=7.0 Hz), 1.30-1.82 (4H, m), 1.37 (9H, brs), 2.73 (3H, s), 2.98 (2H, J=6.4 Hz, q), 4.15 (1H, td, J=8.4, 4.8 Hz), 4.34-4.66 (1H, m), 5.00 (2H, s), 7.21-7.42 (6H, m), 7.94 (1H, d, J=7.7 Hz), 12.55 (1H, brs).

### (ii) Production of tert-butyl(3S,8aR)-3-[(1R)-1,2,3,4-tetrahydronaphthalen-1-ylcarbamoyl]hexahydropyrrolo[1,2-a]pyrazine-2(1H)-carboxylate

To a solution of 2-tert-butyl 3-methyl(3S,8aR)-hexahydropyrrolo[1,2-a]pyrazine-2,3(1H)-dicarboxylate (3.00 g) synthesized in the above-mentioned Example 4 (i) in tetrahydrofuran (20 mL) was added an aqueous solution (10 mL) of lithium hydroxide monohydrate (576 mg) at room temperature, and the reaction mixture was stirred at 50° C. for 3 hr. The reaction mixture was allowed to cool to room temperature, neutralized with 1N hydrochloric acid (13.7 mL), and concentrated. The residue was dissolved in tetrahydrofuran, (1R)-1,2,3,4-tetrahydronaphthalen-1-amine (2.33 mL) and 4-(4,6-dimethoxy-1,3,5-triazin-2-yl)-4-methylmorpholinium chloride (4.37 g) were added thereto under ice-cooling, and the reaction mixture was stirred at room temperature for 14 hr. The mixture was diluted with ethyl acetate (200 mL), and washed with water (50 mL) and saturated brine (50 mL). The organic layer was dried over anhydrous magnesium sulfate, and concentrated under reduced pressure. The residue was purified by silica gel column chromatography (ethyl acetate/hexane=100/0→50/50) to give the title compound (1.79 g) as a colorless amorphous powder.

¹H NMR (300 MHz, DMSO-d₆): δ 1.14-1.38 (1H, m), 1.35-1.45 (9H, m), 1.56-2.04 (9H, m), 2.10-2.26 (1H, m), 2.62-2.81 (2H, m), 2.83-3.07 (2H, m), 3.28-3.49 (1H, m), 3.81-3.94 (1H, m), 4.30-4.55 (1H, m), 4.88-5.06 (1H, m), 7.04-7.23 (4H, m), 8.02-8.33 (1H, m).

### (iii) Production of (3S,8aR)—N-[(1R)-1,2,3,4-tetrahydronaphthalen-1-yl]octahydropyrrolo[1,2-a]pyrazine-3-carboxamide dihydrochloride

tert-Butyl(3S,8aR)-3-[(1R)-1,2,3,4-tetrahydronaphthalen-1-ylcarbamoyl]hexahydropyrrolo[1,2-a]pyrazine-2(1H)-carboxylate (950 mg) was dissolved in 4M hydrogen chloride-ethyl acetate solution, and the solution was stirred at room temperature for 1 hr, and concentrated. The residue was collected by filtration, washed with ethyl acetate and methanol, and dried under reduced pressure to give the title compound (797 mg) as a colorless amorphous powder.

¹H NMR (300 MHz, DMSO-d₆): δ 1.59-2.25 (8H, m), 2.62-2.86 (2H, m), 3.05-4.09 (9H, m), 4.21-4.43 (1H, m), 5.02 (1H, d, J=5.7 Hz), 6.94-7.40 (4H, m), 9.28 (1H, brs), 12.42 (1H, brs).

### (iv) Production of benzyl{(4S)-4-[(N-methyl-L-alanyl)amino]-5-oxo-5-[(3S,8aR)-3-[(1R)-1,2,3,4-tetrahydronaphthalen-1-ylcarbamoyl]hexahydropyrrolo[1,2-a]pyrazin-2(1H)-yl]pentyl}carbamate dihydrochloride

N-(tert-Butoxycarbonyl)-N-methyl-L-alanyl-N-5-[(benzyloxy)carbonyl]-L-ornithine (146 mg), (3S,8aR)—N-[(1R)-1,2,3,4-tetrahydronaphthalen-1-yl]octahydropyrrolo[1,2-a]pyrazine-3-carboxamide dihydrochloride (100 mg), 4-(4,6-dimethoxy-1,3,5-triazin-2-yl)-4-methylmorpholinium chloride (89 mg) and 4-methylmorpholine (0.074 mL) were mixed in tetrahydrofuran (3 mL) under ice-cooling, and the mixture was allowed to warm to room temperature, and stirred for 6 hr. The mixture was diluted with ethyl acetate (50 mL), and washed with water (25 mL) and saturated brine (25 mL). The organic layer was dried over anhydrous magnesium sulfate, and concentrated under reduced pressure. The residue was subjected to silica gel column chromatography (ethyl acetate/hexane=10/90→10/90), and the collected fractions were concentrated to give a colorless amorphous powder. This amorphous powder was dissolved in 4M hydrogen chloride-ethyl acetate solution (3 mL), and the solution was stirred at room temperature for 10 min, and concentrated under reduced pressure. The residue was collected by filtration, washed with ethyl acetate and diethyl ether, and dried under reduced pressure to give the title compound (170 mg) as a colorless amorphous powder.

¹H NMR (300 MHz, DMSO-d₆): δ 1.39 (3H, d, J=6.6 Hz), 1.47-1.89 (11H, m), 1.96-2.17 (3H, m), 2.44-2.48 (3H, m), 2.72 (2H, d, J=2.5 Hz), 2.94-3.12 (3H, m), 3.21-3.94 (5H, m), 4.35 (1H, d, J=13.2 Hz), 4.62 (1H, dd, J=11.4, 5.4 Hz), 4.84-5.08 (4H, m), 7.05-7.42 (9H, m), 8.69 (1H, d, J=8.5 Hz), 8.90 (2H, d, J=7.7 Hz), 9.33 (1H, brs), 11.96 (1H, brs).

### Example 6

### Production of benzyl{(5S)-5-[(N-methyl-L-alanyl)amino]-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}carbamate dihydrochloride

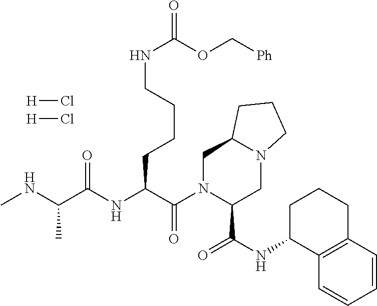

### (i) Production of N-(tert-butoxycarbonyl)-N-methyl-L-alanyl-N-6-[(benzyloxy)carbonyl]-L-lysin

N-(tert-Butoxycarbonyl)-N-methyl-L-alanine (5.0 g), methyl N-6-[(benzyloxy)carbonyl]-L-lysinate hydrochloride (11.4 g), 1-ethyl-3-(3-dimethylaminopropyl)carbodiimide hydrochloride (9.5 g), 1-hydroxybenzotriazole (5.0 g), 4-methylmorpholine (12.0 mL) and 1-methyl-2-pyrrolidone (6.0 mL) were mixed in tetrahydrofuran (100 mL), and the mixture was stirred at room temperature for 5 hr. The mixture was diluted with ethyl acetate (300 mL), and washed with 1N hydrochloric acid (50 mL), saturated aqueous sodium hydrogen carbonate solution (50 mL) and saturated brine (50 mL). The organic layer was dried over anhydrous magnesium sulfate, and concentrated under reduced pressure. The residue was subjected to silica gel column chromatography (ethyl acetate/hexane=10/90→40/60), and the collected fractions were concentrated to give a colorless oil (9.50 g). This colorless oil was dissolved in tetrahydrofuran (40 mL), methanol (25 mL) and water (40 mL), and an aqueous solution (40 mL) of lithium hydroxide monohydrate (2.6 g) was added thereto under ice-cooling. The reaction mixture was stirred at room temperature for 12 hr, and 1N hydrochloric acid (70 mL) was added thereto. The mixture was extracted with ethyl acetate (300 mL), and the organic layer was washed with water (50 mL) and saturated brine (50 mL), dried over anhydrous magnesium sulfate. The insoluble material was filtered off. The filtrate was concentrated, and dried under reduced pressure to give the title compound (9.00 g) as a colorless oil.

¹H NMR (300 MHz, CDCl₃): δ 1.29-1.40 (5H, m), 1.46 (9H, s), 1.52 (2H, m), 1.72 (1H, m), 1.88 (1H, m), 2.77 (3H, s), 3.17 (2H, m), 4.57 (1H, m), 4.71 (1H, brs), 4.94-5.19 (3H, m), 6.76-7.00 (1H, m), 7.29-7.41 (5H, m).

### (ii) Production of benzyl{(5S)-5-({(2S)-2-[(tert-butoxycarbonyl)(methyl)amino]propanoyl}amino)-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}carbamate

N-(tert-Butoxycarbonyl)-N-methyl-L-alanyl-N-6-[(benzyloxy)carbonyl]-L-lysin (615 mg), (3S,8aR)—N-[(1R)-1,2,3,4-tetrahydronaphthalen-1-yl]octahydropyrrolo[1,2-a]pyrazine-3-carboxamide dihydrochloride (410 mg), 4-(4,6-dimethoxy-1,3,5-triazin-2-yl)-4-methylmorpholinium chloride (365 mg) and 4-methylmorpholine (0.303 mL) were mixed in tetrahydrofuran (3 mL) under ice-cooling, and the mixture was allowed to warm to room temperature, and stirred for 6 hr. The mixture was diluted with ethyl acetate (200 mL), and washed with water (50 mL) and saturated brine (50 mL). The organic layer was dried over anhydrous magnesium sulfate, and concentrated under reduced pressure. The residue was subjected to silica gel column chromatography (ethyl acetate/hexane=10/90→10/90), and the collected fractions were concentrated to give the title compound as a colorless amorphous powder. LC-MS: 747 (MH⁺).

### (iii) Production of benzyl{(5S)-5-[(N-methyl-L-alanyl)amino]-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}carbamate dihydrochloride

Benzyl{(55)-5-({(2S)-2-[(tert-butoxycarbonyl)(methyl)amino]propanoyl}amino)-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}carbamate (170 mg) was dissolved in 4M hydrogen chloride-ethyl acetate solution (3 mL), and the solution was stirred at room temperature for 10 min. The mixture was concentrated under reduced pressure, and the residue was collected by filtration, washed with ethyl acetate and diethyl ether, and dried under reduced pressure to give the title compound (165 mg) as a colorless amorphous powder.

¹H NMR (300 MHz, DMSO-d₆): δ 1.29-1.48 (5H, m), 1.57-2.22 (14H, m), 2.45-2.50 (3H, m), 2.64-2.80 (2H, m), 2.90-3.17 (3H, m), 3.46-3.93 (6H, m), 4.26-4.43 (1H, m), 4.54-4.67 (1H, m), 4.82-5.08 (3H, m), 7.02-7.41 (9H, m), 8.63-9.02 (3H, m), 9.17 (1H, brs), 11.72 (1H, brs).

### Example 7

### Production of (3S,8aR)-2-(N-methyl-L-alanyl-N-6-acetyl-L-lysyl)-N-[(1R)-1,2,3,4-tetrahydronaphthalen-1-yl]octahydropyrrolo[1,2-a]pyrazine-3-carboxamide dihydrochloride

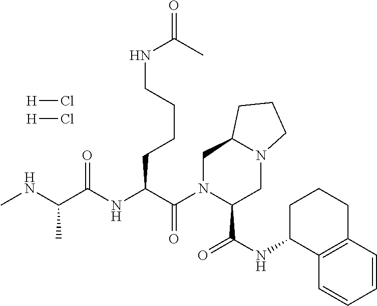

### (i) Production of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate

N-(tert-Butoxycarbonyl)-N-methyl-L-alanyl-N-6-[(benzyloxy)carbonyl]-L-lysin (1.17 g), (3S,8aR)—N-[(1R)-1,2,3,4-tetrahydronaphthalen-1-yl]octahydropyrrolo[1,2-a]pyrazine-3-carboxamide dihydrochloride (780 mg), 4-(4,6-dimethoxy-1,3,5-triazin-2-yl)-4-methylmorpholinium chloride (695 mg) and 4-methylmorpholine (0.576 mL) were mixed in tetrahydrofuran (15 mL) under ice-cooling, and the mixture was allowed to warm to room temperature, and stirred for 6 hr. The mixture was diluted with ethyl acetate (300 mL), and washed with water (50 mL) and saturated brine (50 mL). The organic layer was dried over anhydrous magnesium sulfate, and concentrated under reduced pressure. The residue was subjected to silica gel column chromatography (ethyl acetate/hexane=10/90→10/90), and the collected fractions were concentrated to give a colorless amorphous powder. This amorphous powder was dissolved in methanol (20 mL), 10% palladium-carbon (100 mg) was thereto, and the mixture was stirred at room temperature for 4 hr under a hydrogen atmosphere (1 atm). The insoluble material was filtered off through a celite pad, and the filtrate was concentrated to give the title compound (1.02 g) as a colorless oil. LC-MS: 613 (MH⁺).

### (ii) Production of (3S,8aR)-2-(N-methyl-L-alanyl-N-6-acetyl-L-lysyl)-N-[(1R)-1,2,3,4-tetrahydronaphthalen-1-yl]octahydropyrrolo[1,2-a]pyrazine-3-carboxamide dihydrochloride

To a solution of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate (200 mg) in N,N-dimethylformamide (5 mL) was added acetic anhydride (0.037 mL), and the mixture was stirred at room temperature for 2 hr. To this reaction mixture was added water (25 mL), and the mixture was extracted with ethyl acetate (100 mL). The organic layer was washed with saturated brine (25 mL), dried over anhydrous magnesium sulfate, and concentrated. The residue was subjected to silica gel column chromatography (ethyl acetate/methanol=100/0→90/10), and the collected fractions were concentrated to give a colorless oil. This oil was dissolved in 4M hydrogen chloride-ethyl acetate solution (3 mL), and the mixture was stirred at room temperature for 10 min, and concentrated under reduced pressure. The residue was collected by filtration, washed with ethyl acetate and diethyl ether, and dried under reduced pressure to give the title compound (76 mg) as a colorless amorphous powder.

¹H NMR (300 MHz, DMSO-d₆): δ 1.30-1.47 (6H, m), 1.54-1.89 (9H, m), 1.95-2.18 (2H, m), 2.47 (3H, t, J=4.6 Hz), 2.61-2.83 (2H, m), 2.95-3.14 (3H, m), 3.33-3.80 (4H, m), 3.87 (1H, d, J=5.5 Hz), 4.30-4.48 (1H, m), 4.56-4.70 (1H, m), 4.82-4.98 (2H, m), 5.29-5.78 (4H, m), 7.02-7.27 (4H, m), 7.96 (1H, brs), 8.72 (1H, d, J=8.5 Hz), 8.85-9.09 (2H, m), 9.53 (1H, brs), 11.98-12.52 (1H, m).

### Example 8

### Production of (3S,8aR)-2-[N-methyl-L-alanyl-N-6-(phenylsulfonyl)-L-lysyl]-N-[(1R)-1,2,3,4-tetrahydronaphthalen-1-yl]octahydropyrrolo[1,2-a]pyrazine-3-carboxamide dihydrochloride

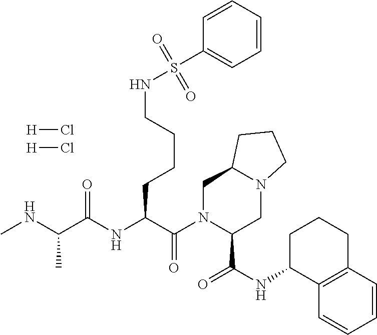

To a solution of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate (200 mg) obtained in Example 7 (i) in N,N-dimethylformamide (2 mL) were added benzenesulfonyl chloride (0.050 mL) and pyridine (0.032 mL), and the mixture was stirred at room temperature for 3 hr. To the reaction mixture was added water (25 mL), and the mixture was extracted with ethyl acetate (100 mL). The organic layer was washed with saturated brine (25 mL), dried over anhydrous magnesium sulfate, and concentrated. The residue was subjected to silica gel column chromatography (ethyl acetate/methanol=100/0→90/10), and the collected fractions were concentrated to give a colorless oil. This oil was dissolved in 2M hydrogen chloride-ethyl acetate solution (3 mL), and the mixture was stirred at room temperature for 2 hr, and concentrated under reduced pressure. The residue was collected by filtration, washed with ethyl acetate and diethyl ether, and dried under reduced pressure to give the title compound (58 mg) as a colorless amorphous powder.

¹H NMR (300 MHz, DMSO-d₆): δ 1.28-1.55 (4H, m), 1.37 (3H, t, J=7.1 Hz), 1.59-1.90 (7H, m), 2.03-2.18 (2H, m), 2.44-2.49 (3H, m), 2.64-2.78 (3H, m), 3.06 (1H, brs), 3.46-3.90 (7H, m), 4.27-4.38 (1H, m), 4.61 (1H, dd, J=11.7, 5.3 Hz), 4.79-4.95 (2H, m), 7.05-7.23 (4H, m), 7.51-7.68 (4H, m), 7.72-7.82 (2H, m), 8.64-8.99 (3H, m), 9.21 (2H, brs), 11.82 (1H, brs).

### Example 9

### Production of (3S,8aR)-2-[N-methyl-L-alanyl-N-6-(3-phenylpropanoyl)-L-lysyl]-N-[(1R)-1,2,3,4-tetrahydronaphthalen-1-yl]octahydropyrrolo[1,2-a]pyrazine-3-carboxamide dihydrochloride

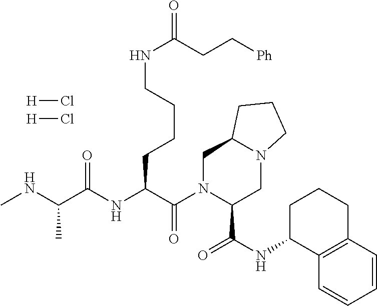

To a solution of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate (200 mg) obtained in Example 7 (i) in N,N-dimethylformamide (2 mL) were added 3-phenylpropanoyl chloride (0.058 mL) and pyridine (0.032 mL), and the mixture was stirred at room temperature for 3 hr. To the reaction mixture was added water (25 mL), and the mixture was extracted with ethyl acetate (100 mL). The organic layer was washed with saturated brine (25 mL), dried over anhydrous magnesium sulfate, and concentrated. The residue was subjected to silica gel column chromatography (ethyl acetate/methanol=100/0→90/10), and the collected fractions were concentrated to give a colorless oil. This oil was dissolved in 2M hydrogen chloride-ethyl acetate solution (3 mL), and the mixture was stirred at room temperature for 2 hr, and concentrated under reduced pressure. The residue was collected by filtration, washed with ethyl acetate and diethyl ether, and dried under reduced pressure to give the title compound (113 mg) as a colorless amorphous powder.

¹H NMR (300 MHz, DMSO-d₆): δ 1.32-1.44 (7H, m), 1.54-1.87 (7H, m), 2.01-2.17 (2H, m), 2.36 (2H, t, J=7.8 Hz), 2.44-2.48 (3H, m), 2.65-2.85 (4H, m), 2.94-3.17 (4H, m), 3.49-3.92 (6H, m), 4.36 (1H, d, J=11.0 Hz), 4.63 (1H, m), 4.82-4.95 (2H, m), 7.00-7.35 (9H, m), 7.82-8.03 (1H, m), 8.73 (1H, d, J=8.5 Hz), 8.84-9.06 (2H, m), 9.37 (1H, brs), 11.76-12.57 (1H, m).

### Example 10

### Production of (3S,8aR)-2-[(2S)-2-[(N-methyl-L-alanyl)amino]-2-(tetrahydro-2H-pyran-4-yl)acetyl]-N-[(1R)-1,2,3,4-tetrahydronaphthalen-1-yl]octahydropyrrolo[1,2-a]pyrazine-3-carboxamide dihydrochloride

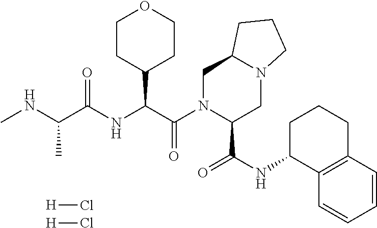

tert-Butyl(3S,8aR)-3-[(1R)-1,2,3,4-tetrahydronaphthalen-1-ylcarbamoyl]hexahydropyrrolo[1,2-a]pyrazine-2(1H)-carboxylate (380 mg) obtained in Example 5 (ii) was dissolved in 4M hydrogen chloride-ethyl acetate solution (5 mL), and the solution was stirred at room temperature for 18 hr. The precipitate resulting from the reaction mixture was collected by filtration. To the collected precipitate were added tetrahydrofuran (5 mL), N-ethyl-N-(1-methylethyl)propan-2-amine (0.497 mL), (2S)-{[(benzyloxy)carbonyl]amino}(tetrahydro-2H-pyran-4-yl)ethanoic acid (418 mg) and 1-hydroxybenzotriazole (218 mg). To this mixture was added 1-ethyl-3-3-dimethylaminopropyl)carbodiimide hydrochloride (310 mg), and the mixture was stirred at room temperature for 18 hr. To the reaction mixture was added water (50 mL), and the mixture was extracted with ethyl acetate (200 mL). The organic layer was washed with saturated brine (50 mL), dried over anhydrous magnesium sulfate, and filtered, and the filtrate was concentrated to give an oil. This oil (500 mg) was dissolved in methanol (3 mL), 20% palladium-carbon (100 mg, 20 wt %) was added thereto, and the mixture was stirred at room temperature for 4 hr under a hydrogen atmosphere (3 atm). The insoluble material was filtered off through a celite pad, and the filtrate was concentrated to give an oil.

To this oil were added N,N-dimethylformamide (3 mL), N-ethyl-N-(1-methylethyl)propan-2-amine (0.379 mL) and N-(tert-butoxycarbonyl)-N-methyl-L-alanine (265 mg). To the mixture was added O-(1H-6-chlorobenzotriazol-1-yl)-N,N,N′,N′-tetramethyluronium hexafluorophosphate (612 mg), and the reaction mixture was stirred at room temperature for 3 hr. To the reaction mixture was added water (50 mL), and the mixture was extracted with ethyl acetate (200 mL). The organic layer was washed with saturated brine (50 mL), dried over anhydrous magnesium sulfate, and filtered, and the filtrate was concentrated. The residue was subjected to silica gel column chromatography (hexane/ethyl acetate=90/10→0/100), and the collected fractions were concentrated to give an oil. This oil was dissolved in 4M hydrogen chloride-ethyl acetate solution (5 mL), and the solution was stirred at room temperature for 30 min. To the reaction solution was added diethyl ether (20 mL), and the precipitate was collected by filtration, and dried under reduced pressure to give the title compound (150 mg) as a colorless amorphous powder.

¹H NMR (300 MHz, DMSO-d₆): δ 1.25-1.45 (5H, m), 1.55-1.92 (8H, m), 1.95-2.17 (3H, m), 2.45-2.49 (3H, m), 2.63-2.81 (2H, m), 2.95-3.14 (1H, m), 3.26 (2H, t, J=11.7 Hz), 3.46-3.97 (7H, m), 4.28 (1H, brs), 4.47-4.69 (2H, m), 4.88 (2H, dt, J=15.5, 7.6 Hz), 7.02-7.26 (4H, m), 8.70-8.96 (3H, m), 9.30 (1H, brs), 11.97 (1H, brs).

### Example 11

### Production of (3S,8aS)-2-{(2S)-2-cyclohexyl-2-[(N-methyl-L-alanyl)amino]acetyl}-N-[(1R)-1,2,3,4-tetrahydronaphthalen-1-yl]octahydropyrrolo[1,2-a]pyrazine-3-carboxamide dihydrochloride

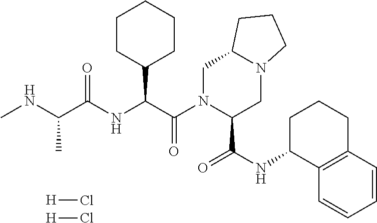

### (i) Production of N-benzyl-5-oxo-L-prolinamide

5-oxo-L-proline (5.0 g), benzylamine (5.08 mL) and O-(7-azabenzotriazol-1-yl)-N,N,N′,N′-tetramethyluronium hexafluorophosphate (14.7 g) were mixed in N,N-dimethylformamide at room temperature, and the mixture was stirred for 18 hr. The mixture was diluted with ethyl acetate (300 mL), and washed with saturated aqueous citric acid solution (50 mL), saturated aqueous sodium hydrogen carbonate solution (50 mL) and saturated brine (50 mL). The organic layer was dried over anhydrous magnesium sulfate, and concentrated under reduced pressure. The residue was subjected to silica gel column chromatography (ethyl acetate/methanol=100/0→90/10), and the collected fractions were concentrated to give the title compound (3.75 g) as a colorless amorphous powder.

¹H NMR (300 MHz, DMSO-d₆): δ 1.81-1.97 (1H, m), 2.01-2.36 (3H, m), 4.04 (1H, m), 4.29 (2H, d, J=5.9 Hz), 7.18-7.37 (5H, m), 7.85 (1H, s), 8.50 (1H, t, J=5.9 Hz).

### (ii) Production of 1-phenyl-N-[(2S)-pyrrolidin-2-ylmethyl]methanamine

To a suspension of lithium aluminum hydride (2.56 g) in tetrahydrofuran (50 mL) was added dropwise a suspension (25 mL) of N-benzyl-5-oxo-L-prolinamide (4.90 g) in tetrahydrofuran under ice-cooling, and the mixture was stirred with heating at 60° C. for 18 hr. The reaction mixture was cooled to 0° C., water (2.6 mL), 1N aqueous sodium hydroxide solution (2.6 mL) and water (3.6 mL) were successively added thereto, and the resulting insoluble material was filtered off. The filtrate was concentrated under reduced pressure to give the title compound (3.85 g) as a pale-yellow oil. Without further purification, the compound was used for the next reaction. LC-MS: 191 (MH⁺).

### (iii) Production of 2-tert-butyl 3-methyl(3S,8aS)-hexahydropyrrolo[1,2-a]pyrazine-2,3(1H)-dicarboxylate

To a suspension of 1-phenyl-N-[(2S)-pyrrolidin-2-ylmethyl]methanamine (3.85 g) in toluene (25 mL) were added triethylamine (6.49 mL) and a solution (50 mL) of methyl 2,3-dibromopropanoate (4.48 g) in toluene, and the mixture was stirred with heating at 90° C. for 5 hr. The reaction mixture was allowed to cool to room temperature, and the insoluble material was filtered off. To the filtrate was added water (100 mL), and the mixture was extracted with ethyl acetate (300 mL). The organic layer was washed with saturated brine (100 mL), dried over anhydrous magnesium sulfate, and concentrated to give a pale-yellow oil (5.00 g). To a solution of this oil in 5-10% hydrogen chloride-methanol was added 10% palladium-carbon (500 mg, 50% wet), and the mixture was stirred at room temperature for 3 hr under a hydrogen atmosphere. The insoluble material was filtered off through a celite pad, and the filtrate was concentrated to give a pale-yellow oil. This oil was dissolved in saturated aqueous sodium hydrogen carbonate solution (50 mL) and tetrahydrofuran (50 di-tert-butyl bicarbonate (4.17 g) was added thereto, and the mixture was stirred at room temperature for 18 hr. The mixture was diluted with ethyl acetate (300 mL), and washed with water (50 mL) and saturated brine (50 mL). The organic layer was dried over anhydrous magnesium sulfate, and concentrated under reduced pressure. The residue was purified by silica gel column chromatography (ethyl acetate/hexane=10/90→ethyl acetate) to give the title compound (2.11 g) as a colorless oil.

¹H NMR (300 MHz, DMSO-d₆): δ 1.27-1.45 (1H, m), 1.36 (9H, s), 1.52-1.67 (1H, m), 1.68-1.89 (2H, m), 2.56-2.65 (2H, m), 2.75-2.99 (2H, m), 3.12 (1H, dd, J=12.8, 7.7 Hz), 3.38 (1H, q, J=6.9 Hz), 3.54-3.69 (4H, m), 4.29 (1H, t, J=5.0 Hz).

### (iv) Production of tert-butyl(3S,8aS)-3-[(1R)-1,2,3,4-tetrahydronaphthalen-1-ylcarbamoyl]hexahydropyrrolo[1,2-a]pyrazine-2(1H)-carboxylate

To a solution of 2-tert-butyl 3-methyl(3S,8aS)-hexahydropyrrolo[1,2-a]pyrazine-2,3(1H)-dicarboxylate (1.00 g) in tetrahydrofuran (15 mL) was added an aqueous solution (5 mL) of lithium hydroxide monohydrate (221 mg) at room temperature, and the reaction mixture was stirred at 50° C. for 3 hr. This reaction mixture was allowed to cool to room temperature, neutralized with 1M hydrochloric acid (5.28 mL), and concentrated. The residue was dissolved in tetrahydrofuran, (1R)-1,2,3,4-tetrahydronaphthalen-1-amine (0.78 mL), 1-ethyl-3-(3-dimethylaminopropyl)carbodiimide hydrochloride (4.37 g) and 1-hydroxybenzotriazole (951 mg) were added thereto. The reaction mixture was stirred at room temperature for 18 hr, water (50 mL) was added thereto, and the mixture was extracted with ethyl acetate (300 mL). The organic layer was washed with saturated brine (50 mL), dried over anhydrous magnesium sulfate, and filtered, and the filtrate was concentrated to give an oil. The oil was subjected to silica gel column chromatography (ethyl acetate/methanol=100/0→70/30), and the collected fractions were concentrated to give the title compound (900 mg) as a colorless amorphous powder.

¹H NMR (300 MHz, DMSO-d₆): δ 1.16-1.35 (1H, m), 1.36-1.45 (9H, m), 1.55-2.05 (9H, m), 2.10-2.27 (1H, m), 2.64-3.07 (4H, m), 3.28-3.49 (1H, m), 3.90 (1H, m), 4.33-4.55 (1H, m), 4.90-5.06 (1H, m), 6.99-7.28 (4H, m), 7.99-8.34 (1H, m).

### (v) Production of (3S,8aS)-2-{(2S)-2-cyclohexyl-2-[(N-methyl-L-alanyl)amino]acetyl}-N-[(1R)-1,2,3,4-tetrahydronaphthalen-1-yl]octahydropyrrolo[1,2-a]pyrazine-3-carboxamide dihydrochloride

tert-Butyl(3S,8aS)-3-[(1R)-1,2,3,4-tetrahydronaphthalen-1-ylcarbamoyl]hexahydropyrrolo[1,2-a]pyrazine-2(1H)-carboxylate (380 mg) was dissolved in 4M hydrogen chloride-ethyl acetate solution (5 mL), and the solution was stirred at room temperature for 18 hr. The precipitate resulting from the reaction mixture was collected by filtration. The collected precipitate, (2S)-{[N-(tert-butoxycarbonyl)-N-methyl-L-alanyl]amino}(cyclohexyl)ethanoic acid (1.35 g), 4-(4,6-dimethoxy-1,3,5-triazin-2-yl)-4-methylmorpholinium chloride (1.25 g) and 4-methylmorpholine (0.742 mL) were mixed in tetrahydrofuran (10 mL), and the mixture was stirred at room temperature for 5 hr. The mixture was diluted with ethyl acetate (250 mL), and washed with water (50 mL) and saturated brine (50 mL). The organic layer was dried over anhydrous magnesium sulfate, and concentrated under reduced pressure.

The residue was subjected to silica gel column chromatography (ethyl acetate/hexane=10/90→100/0, and then ethyl acetate/methanol=90/10), and the collected fractions were concentrated to give a colorless amorphous powder. This amorphous powder was dissolved in 4M hydrogen chloride-ethyl acetate solution (10 mL), and the solution was stirred at room temperature for 30 min. To the reaction solution was added diethyl ether (20 mL), and the precipitate was collected by filtration, and dried under reduced pressure to give the title compound (451 mg) as a colorless amorphous powder.

¹H NMR (300 MHz, DMSO-d₆): δ 0.98-1.24 (5H, m), 1.27-1.44 (3H, m), 1.53-2.06 (13H, m), 2.18 (1H, brs), 2.46 (3H, brs), 2.62-2.86 (2H, m), 3.08 (1H, brs), 3.49-3.73 (3H, m), 3.79-4.37 (4H, m), 4.49-4.76 (2H, m), 4.85-5.06 (1H, m), 7.04-7.27 (4H, m), 8.50-9.10 (3H, m), 9.56 (1H, brs), 11.17 (1H, brs).

### Example 12

### Production of N-{(1S)-1-cyclohexyl-2-[(3S,8aR)-3-{4-[(4-fluorophenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

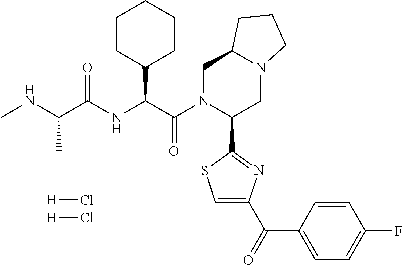

### (i) Production of tert-butyl(3S,8aR)-3-carbamoylhexahydropyrrolo[1,2-a]pyrazine-2(1H)-carboxylate

To a solution of 2-tert-butyl 3-methyl(3S,8aR)-hexahydropyrrolo[1,2-a]pyrazine-2,3(1H)-dicarboxylate (1.93 g) synthesized in Example 4 (i) in a mixed solvent of tetrahydrofuran/water (4/1, 35 mL) was added lithium hydroxide monohydrate (370 mg), and the mixture was stirred with heating at 50° C. for 5 hr. The reaction mixture was allowed to cool to room temperature, neutralized with 1N hydrochloric acid (8.0 mL), and concentrated under reduced pressure. The obtained residue was dissolved in N,N-dimethylformamide (34 mL), 1-hydroxybenzotriazole ammonium salt (1.55 g) and N-[3-(dimethylamino)propyl]-N′-ethylcarbodiimide hydrochloride (1.96 g) were added thereto, and the mixture was stirred at room temperature for 5 hr. The solvent was evaporated under reduced pressure, and the obtained residue was diluted with ethyl acetate (100 mL) and saturated aqueous sodium hydrogen carbonate solution (100 mL). The aqueous layer was extracted with ethyl acetate (100 mL), the combined organic layers were dried over anhydrous magnesium sulfate, and the insoluble material was filtered off. The filtrate was concentrated under reduced pressure, and the residue was purified by silica gel column chromatography (ethyl acetate/hexane-30/70→100/0) to give the title compound (1.27 g) as a colorless amorphous powder.

¹H NMR (DMSO-d₆, 300 MHz): δ 1.12-1.31 (1H, m), 1.38 (9H, d, J=11.9 Hz), 1.55-1.83 (4H, m), 1.88-2.01 (1H, m), 2.09-2.19 (1H, m), 2.64-2.99 (2H, m), 3.41 (1H, dd, J=8.4, 10.1 Hz), 3.79-3.94 (1H, m), 4.29-4.51 (1H, m), 6.95 (1H, d, J=10.6 Hz), 7.26 (1H, d, J=5.3 Hz).

### (ii) Production of tert-butyl(3S,8aR)-3-carbamothioylhexahydropyrrolo[1,2-a]pyrazine-2(1H)-carboxylate

To a solution of tert-butyl(3S,8aR)-3-carbamoylhexahydropyrrolo[1,2-a]pyrazine-2(1H)-carboxylate (1.25 g) in tetrahydrofuran (23.5 mL) was added 2,4-bis(4-methoxyphenyl)-1,3,2,4-dithiadiphosphetane-2,4-disulfide (1.90 g), and the mixture was heated under reflux at 80° C. for 12 hr. The reaction mixture was allowed to cool to room temperature, the solvent was evaporated under reduced pressure, and the obtained residue was purified by silica gel column chromatography (ethyl acetate/hexane=20/80→100/0) and then basic silica gel column chromatography (ethyl acetate/hexane=30/70→100/0) to give the title compound (290 mg) as a colorless solid.

¹H NMR (DMSO-d₆, 300 MHz): δ 1.14-1.32 (1H, m), 1.39 (9H, d, J=11.0 Hz), 1.56-1.84 (4H, m), 1.88-2.01 (1H, m), 2.14-2.30 (1H, m), 2.82-3.10 (2H, m), 3.63-3.82 (1H, m), 3.86-4.06 (1H, m), 4.59 (1H, dd, J=3.6, 13.8 Hz), 8.92 (1H, d, J=17.2 Hz), 9.64 (1H, brs).

### (iii) Production of tert-butyl(3S,8aR)-3-[4-(ethoxycarbonyl)-1,3-thiazol-2-yl]hexahydropyrrolo[1,2-a]pyrazine-2(1H)-carboxylate

To a solution of tert-butyl(3S,8aR)-3-carbamothioylhexahydropyrrolo[1,2-a]pyrazine-2(1H)-carboxylate (285 mg) in dimethoxyethane (10 mL) were added potassium hydrogen carbonate (801 mg) and ethyl 3-bromo-2-oxopropanoate (585 mg), and the mixture was stirred at room temperature for 2 hr. The reaction mixture was cooled to 0° C., trifluoroacetic anhydride (700 μL) and 2,4,6-trimethylpyridine (210 μL) were added thereto. The mixture was allowed to warm to room temperature, and stirred for 17 hr. The reaction mixture was diluted with water (30 mL) and ethyl acetate (40 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→50/50) to give the title compound (261 mg) as a pale-yellow oil.

¹H NMR (DMSO-d₆, 300 MHz): δ 1.22-1.33 (4H, m), 1.35-1.49 (9H, m), 1.58-1.73 (2H, m), 1.75-1.86 (1H, m), 1.87-1.98 (1H, m), 2.06 (1H, dd, J=7.6, 8.9 Hz), 2.40-2.46 (1H, m), 2.57-2.87 (1H, m), 2.90-3.00 (1H, m), 3.47-3.62 (1H, m), 4.06 (1H, dd, J=2.1 Hz, 11.9 Hz), 4.29 (2H, q, J=7.2 Hz), 5.42 (1H, d, J=16.8 Hz), 8.43 (1H, s).

### (iv) Production of tert-butyl(3S,8aR)-3-[(4-[methoxy(methyl)carbamoyl]-1,3-thiazol-2-yl]hexahydropyrrolo[1,2-a]pyrazine-2(1H)-carboxylate

To a solution of tert-butyl(3S,8aR)-3-[4-(ethoxycarbonyl)-1,3-thiazol-2-yl]hexahydropyrrolo[1,2-a]pyrazine-2(1H)-carboxylate (261 mg) in a mixed solvent (6.25 mL) of tetrahydrofuran/water (4/1) was added lithium hydroxide monohydrate (42.0 mg), and the mixture was stirred with heating at 50° C. for 16 hr. The reaction mixture was allowed to cool to room temperature, neutralized with 1N hydrochloric acid (800 μL), and concentrated under reduced pressure. The obtained residue was dissolved in N,N-dimethylformamide (6.8 mL), N,O-dimethylhydroxyamine hydrochloride (146 mg, 2.0 mmol), 1-hydroxybenzotriazole (135 mg) and N-[3-(dimethylamino)propyl]-N′-ethylcarbodiimide hydrochloride (192 mg) were added thereto, and the mixture was stirred at room temperature for 18 hr. The reaction mixture was diluted with water (30 mL) and ethyl acetate (30 mL), and the aqueous layer was extracted with ethyl acetate (30 mL×2). The combined organic layers were dried over anhydrous magnesium sulfate, and the insoluble material was filtered off. The filtrate was concentrated under reduced pressure, and the residue was purified by silica gel column chromatography (ethyl acetate/hexane-20/80→100/0) to give the title compound (206 mg) as a pale-yellow oil.

¹H NMR (DMSO-d₆, 300 MHz): δ 1.12-1.53 (10H, m), 1.57-1.72 (2H, m), 1.73-1.97 (2H, m), 2.06 (1H, m), 2.41-2.53 (1H, m), 2.61-2.86 (1H, m), 2.90-3.01 (1H, m), 3.32 (3H, s), 3.48-3.69 (1H, m), 3.74 (3H, s), 4.07 (1H, t, J=11.0 Hz), 5.49 (1H, d, J=17.8 Hz), 8.13 (1H, s).

### (v) Production of tert-butyl(3S,8aR)-3-{4-[(4-fluorophenyl)carbonyl]-1,3-thiazol-2-yl}hexahydropyrrolo[1,2-a]pyrazine-2(1H)-carboxylate

A solution of tert-butyl(3S,8aR)-3-{4-[methoxy(methyl)carbamoyl]-1,3-thiazol-2-yl}hexahydropyrrolo[1,2-a]pyrazine-2(1H)-carboxylate (60 mg) in tetrahydrofuran (3 mL) was cooled to −50° C., 0.8M tetrahydrofuran solution (1.8 mL) of bromo(4-fluorophenyl)magnesium was added thereto, and the mixture was warmed to 0° C. and stirred for 2 hr. To the reaction mixture was added saturated aqueous ammonium chloride solution (15 mL), and the mixture was diluted with ethyl acetate (30 mL). The organic layer was washed with water (30 mL) and saturated brine (30 mL), dried over anhydrous magnesium sulfate, and filtered, and the filtrate was concentrated. The residue was subjected to silica gel column chromatography (ethyl acetate/hexane=90/10→60/40), and the collected fractions were concentrated to give the title compound (59 mg) as a pale-yellow oil. LC-MS: 432 (MH⁺).

### (vi) Production of (4-fluorophenyl){2-[(3S,8aR)-octahydropyrrolo[1,2-a]pyrazin-3-yl]-1,3-thiazol-4-yl}methanone dihydrochloride

To tert-Butyl(3S,8aR)-3-{4-[(4-fluorophenyl)carbonyl]-1,3-thiazol-2-yl}hexahydropyrrolo[1,2-a]pyrazine-2(1H)-carboxylate (80 mg) were added ethyl acetate (2 mL) and 4M hydrogen chloride-ethyl acetate solution (2 mL), and the mixture was stirred at room temperature for 2 hr. The mixture was concentrated under reduced pressure to give the title compound (75 mg) as a colorless amorphous powder. LC-MS: 332 (MH⁺).

### (vii) Production of N-{(1S)-1-cyclohexyl-2-[(3S,8aR)-3-[(4-[(4-fluorophenyl)carbonyl]-1,3-thiazol-2-yl]hexahydropyrrolo[1,2-a]pyrazin-2(1H)-yl]-2-oxoethyl}-N-2-methyl-L-alaninamide dihydrochloride

(4-Fluorophenyl){2-[(3S,8aR)-octahydropyrrolo[1,2-a]pyrazin-3-yl]-1,3-thiazol-4-yl}methanone (80 mg), (2S)-{[N-(tert-butoxycarbonyl)-N-methyl-L-alanyl]amino}(cyclohexyl)ethanoic acid (70 mg), 4-(4,6-dimethoxy-1,3,5-triazin-2-yl)-4-methylmorpholinium chloride (61 mg) and 4-methylmorpholine (0.081 mL) were mixed in tetrahydrofuran (5 mL) under ice-cooling, and the mixture was allowed to warm to room temperature, and stirred for 3 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/90→100/0) to give a colorless amorphous powder. To this amorphous powder were added ethyl acetate (2 mL) and 4M hydrogen chloride-ethyl acetate solution (2 mL), Jo and the mixture was stirred at room temperature for 2 hr. The mixture was concentrated under reduced pressure, and the residue was collected by filtration, washed with ether, and dried under reduced pressure to give the title compound (25 mg) as a colorless amorphous powder.

¹H NMR (DMSO-d₆, 300 MHz): δ 1.07 (3H, s), 1.30-1.46 (3H, m), 1.48-2.42 (13H, m), 2.57-3.22 (2H, m), 3.59-4.44 (6H, m), 4.51-5.07 (2H, m), 5.67-6.50 (1H, m), 7.30-7.48 (2H, m), 8.15-8.35 (2H, m), 8.62 (1H, s), 8.64-8.77 (1H, m), 8.86 (1H, d, J=0.8 Hz), 9.24 (1H, brs), 12.11 (1H, brs).

### Example 13

### Production of N-{(1S)-1-cyclohexyl-2-oxo-2-[(3S,8aR)-3-[4-(thiophen-2-ylcarbonyl)-1,3-thiazol-2-yl]hexahydropyrrolo[1,2-a]pyrazin-2(1H)-yl]ethyl}-N-2-methyl-L-alaninamide dihydrochloride

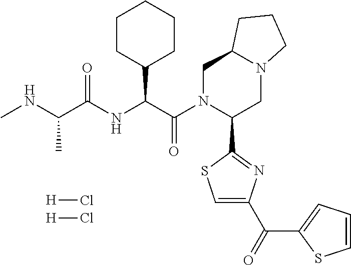

A solution of tert-butyl(3S,8aR)-3-{4-[methoxy(methyl)carbamoyl]-1,3-thiazol-2-yl}hexahydropyrrolo[1,2-a]pyrazine-2(1H)-carboxylate (100 mg) obtained in Example 12 (iv) in tetrahydrofuran was cooled to −50° C., 1M tetrahydrofuran solution (1.26 mL) of bromo(thiophen-2-yl)magnesium was added. The mixture was warmed to 0° C. and stirred for 2 hr. To the reaction mixture was added saturated aqueous ammonium chloride solution (10 mL), and the mixture was diluted with ethyl acetate (100 mL). The organic layer was washed with water (10 mL) and saturated brine (10 mL), dried over anhydrous magnesium sulfate, and filtered, and the filtrate was concentrated to give an oil. The oil was subjected to silica gel column chromatography (ethyl acetate/hexane=5/95→70/30), and the collected fractions were concentrated to give a pale-yellow oil. This oil was dissolved in 4M hydrogen chloride-ethyl acetate solution (1 mL), and the mixture was stirred at room temperature for 1 hr. The mixture was concentrated under reduced pressure, and the residue was washed with ethyl acetate and diethyl ether, dried under reduced pressure to give a colorless amorphous powder. This amorphous powder (60 mg), N-ethyl-N-(1-methylethyl)propan-2-amine (0.107 mL), (2S)-[(tert-butoxycarbonyl)amino](cyclohexyl)ethanoic acid (54.8 mg) and O-(7-azabenzotriazol-1-yl)-N,N,N′,N′-tetramethyluronium hexafluorophosphate (87.5 mg) were mixed in N,N-dimethylformamide (2 mL) at room temperature, and the mixture was stirred for 1 hr. The mixture was diluted with ethyl acetate (100 mL), and washed with saturated brine (20 mL). The organic layer was dried over anhydrous magnesium sulfate, and concentrated under reduced pressure. The residue was subjected to silica gel column chromatography (ethyl acetate/hexane=1/99→70/30), and the collected fractions were concentrated to give a yellow oil. This oil was dissolved in 4M hydrogen chloride-ethyl acetate solution (1 mL), and the mixture was stirred at room temperature for 30 min, and concentrated under reduced pressure. The residue was directly used for the next reaction. The residue obtained in the above-mentioned reaction, N-ethyl-N-(1-methylethyl)propan-2-amine (0.107 mL), N-(tert-butoxycarbonyl)-N-methyl-L-alanine (40.4 mg) and O-(7-azabenzotriazol-1-yl)-N,N,N′,N′-tetramethyluronium hexafluorophosphate (87.5 mg) were mixed in N,N-dimethylformamide (2 mL) at room temperature, and the mixture was stirred for 1 hr. The mixture was diluted with ethyl acetate (100 mL), and washed with saturated brine (20 mL). The organic layer was dried over anhydrous magnesium sulfate, and concentrated under reduced pressure. The residue was subjected to silica gel column chromatography (ethyl acetate/hexane=5/95→70/30), and the collected fractions were concentrated to give a pale-yellow amorphous powder. This amorphous powder was dissolved in 4M hydrogen chloride-ethyl acetate solution (1 mL), and the solution was stirred at room temperature for 30 min. To the reaction solution was added diethyl ether (20 mL), and the precipitate was collected by filtration, and dried to give the title compound (30 mg) as a colorless amorphous powder.

¹H NMR (300 MHz, DMSO-d₆): δ 0.94-1.24 (6H, m), 1.41 (3H, d, J=6.8 Hz), 1.46-2.38 (11H, m), 2.43-2.48 (3H, m), 2.98-3.27 (1H, m), 3.53-4.26 (4H, m), 4.56-5.06 (2H, m), 5.75 (1H, dd, J=11.3, 5.9 Hz), 7.21-7.40 (1H, m), 8.09-8.17 (1H, m), 8.41-8.54 (1H, m), 8.67 (1H, s), 8.78-8.93 (2H, m), 9.14-9.52 (1H, m), 12.15 (1H, brs).

### Example 14

### Production of N-{(1S)-1-cyclohexyl-2-oxo-2-[(3S,8aR)-3-(4-phenyl-1,3-thiazol-2-yl)hexahydropyrrolo[1,2-a]pyrazin-2(1H)-yl]ethyl}-N-2-methyl-L-alaninamide dihydrochloride

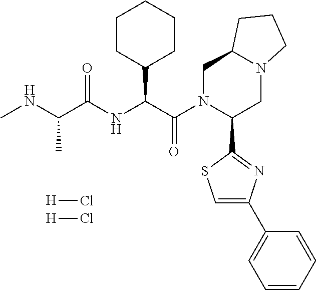

### (i) Production of (3S,8aR)-3-(4-phenyl-1,3-thiazol-2-yl)octahydropyrrolo[1,2-a]pyrazine

A mixture of tert-butyl(3S,8aR)-3-carbamothioylhexahydropyrrolo[1,2-a]pyrazine-2(1H)-carboxylate (230 mg) obtained in Example 12 (ii), 2-bromo-1-phenylethanone (481 mg) and potassium hydrogen carbonate (645 mg) in dimethoxyethane was stirred at room temperature for 1 hr. The reaction mixture was cooled to 0° C., trifluoroacetic anhydride (0.56 mL) and collidine (0.170 mL) were added thereto, and the mixture was allowed to warm to room temperature, and stirred for 3 hr. The mixture was diluted with ethyl acetate (2.00 mL), and washed with water (50 mL) and saturated brine (50 mL). The organic layer was dried over anhydrous magnesium sulfate, and concentrated under reduced pressure. The residue was subjected to basic silica gel column chromatography (ethyl acetate/hexane=0/100→50/50), and the collected fractions were concentrated to give the title compound (125 mg) as a colorless amorphous powder.

¹H NMR (300 MHz, DMSO-d₆): δ 1.06-1.21 (1H, m), 1.23 (1H, s), 1.48-1.63 (2H, m), 1.66-1.91 (2H, m), 1.95-2.08 (1H, m), 2.40 (1H, dd, J=10.8, 4.0 Hz), 2.57 (1H, dd, J=12.4, 10.1 Hz), 2.85-2.97 (2H, m), 3.68 (1H, m), 4.26 (1H, d, J=2.6 Hz), 7.27-7.35 (1H, m), 7.39-7.47 (2H, m), 7.89-7.97 (3H, m).

### (ii) Production of N-{(1S)-1-cyclohexyl-2-oxo-2-[(3S,8aR)-3-(4-phenyl-1,3-thiazol-2-yl)hexahydropyrrolo[1,2-a]pyrazin-2(1H)-yl]ethyl}-N-2-methyl-L-alaninamide dihydrochloride

(3S,8aR)-3-(4-Phenyl-1,3-thiazol-2-yl)octahydropyrrolo[1,2-a]pyrazine (172 mg), (2S)-{[N-(tert-butoxycarbonyl)-N-methyl-L-alanyl]amino}(cyclohexyl)ethanoic acid (110 mg) and 4-(4,6-dimethoxy-1,3,5-triazin-2-yl)-4-methylmorpholinium chloride (138 mg) were mixed in tetrahydrofuran under ice-cooling, and the mixture was allowed to warm to room temperature, and stirred for 3 hr. The mixture was diluted with ethyl acetate (100 mL), and washed with water (25 mL), saturated aqueous sodium hydrogen carbonate solution (25 mL) and saturated brine (25 mL). The organic layer was dried over anhydrous magnesium sulfate, and concentrated under reduced pressure. The residue was subjected to silica gel column chromatography (ethyl acetate/hexane=95/5→40/60), and the collected fractions were concentrated to give a colorless amorphous powder. This amorphous powder was dissolved in 4M hydrogen chloride-ethyl acetate solution (5 mL), and the solution was stirred at room temperature for 30 min. To the reaction solution was added diethyl ether (20 mL), and the precipitate was collected by filtration, and dried to give the title compound (90 mg) as a colorless amorphous powder.

¹H NMR (300 MHz, DMSO-d₆): δ 0.95-1.26 (5H, H), 1.40 (3H, d, J=6.6 Hz), 1.52-2.42 (10H, m), 2.45-2.48 (3H, m), 3.12 (1H, d, J=6.0 Hz), 3.49-3.65 (1H, m), 3.75-4.09 (5H, m), 4.52-5.08 (2H, m), 5.71 (1H, m), 7.28-7.51 (3H, m), 7.88-8.23 (3H, m), 8.67-8.99 (2H, m), 9.23 (1H, brs), 12.03 (1H, brs).

### Example 15

### Production of N-{(1S)-1-cyclohexyl-2-oxo-2-[(3S,8aR)-3-{[(1R)-1,2,3,4-tetrahydronaphthalen-1-ylamino]methyl}hexahydropyrrolo[1,2-a]pyrazin-2(1H)-yl]ethyl}-N-2-methyl-L-alaninamide trihydrochloride

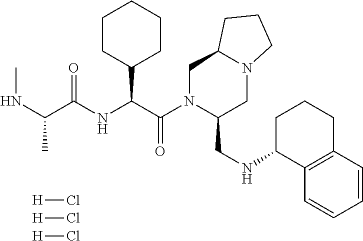

### (i) Production of (3S,8aR)-2-benzyl-N-[(1R)-1,2,3,4-tetrahydronaphthalen-1-yl]octahydropyrrolo[1,2-a]pyrazine-3-carboxamide

To a solution of methyl(3S,8aR)-2-benzyloctahydropyrrolo[1,2-a]pyrazine-3-carboxylate (2.74 g) produced in Example 1 (iii) in a mixed solvent of tetrahydrofuran/water (1/1, 100 mL) was added lithium hydroxide monohydrate (545 mg), and the mixture was stirred with heating at 50° C. for 3 hr. To the reaction mixture was added tetrahydrofuran (50 mL), and the mixture was stirred with heating again at 50° C. for 2 hr. The reaction mixture was allowed to cool, to room temperature, and diluted with diethyl ether (100 mL), and the organic layer was extracted with water (100 mL). The combined aqueous layers were neutralized with 1N hydrochloric acid (13 mL), and the extract was concentrated under reduced pressure. The obtained residue was dissolved in N,N-dimethylformamide (26 mL), (1R)-1,2,3,4-tetrahydronaphthalen-1-amine (1.15 mL), 1-hydroxybenzotriazole (1.06 g) and N-[3-(dimethylamino)propyl]-N′-ethylcarbodiimide hydrochloride (1.50 g) were added thereto, and the mixture was stirred at room temperature for 6 hr. The reaction mixture was diluted with ethyl acetate (200 mL) and water (50 mL), and the aqueous layer was extracted with ethyl acetate (200 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=20/80→100/0) to give the title compound (1.18 g) as a pale-yellow oil.

¹H NMR (DMSO-d₆, 300 MHz): δ 1.08-1.42 (1H, m) 1.51-2.06 (9H, m) 2.27-2.43 (1H, m) 2.61-3.00 (5H, m) 3.12-3.99 (4H, m) 4.90-5.06 (1H, m) 6.94-7.40 (9H, m) 8.17-8.39 (1H, m).

### (ii) Production of (1R)—N-{[(3R,8aR)-2-benzyloctahydropyrrolo[1,2-a]pyrazin-3-yl]methyl}1,2,3,4-tetrahydronaphthalen-1-amine

To a suspension of lithium aluminum hydride (342 mg) in tetrahydrofuran (15 mL) was added a solution of (3S,8aR)-2-benzyl-[(1R)-1,2,3,4-tetrahydronaphthalen-1-yl]octahydropyrrolo[1,2-a]pyrazine-3-carboxamide (1.17 g) in tetrahydrofuran (15 mL) under ice-cooling, and the mixture was stirred with heating at 70° C. for 24 hr. The reaction mixture was allowed to cool to room temperature, water (700 μL), 1N aqueous sodium hydroxide solution (350 μL) and water (350 μL) were successively added thereto, and the resulting insoluble material was filtered off. The filtrate was concentrated under reduced pressure, and the residue was subjected to silica gel column chromatography (ethyl acetate/hexane=20/80→100/0→ethyl acetate/methanol=70:30) to give the title compound (220 mg) as a pale-yellow oil. LC-MS: 376 (MH⁺).

### (iii) Production of tert-butyl{[(3S,8aR)-2-benzyloctahydropyrrolo[1,2-a]pyrazin-3-yl]methyl}(1R)-1,2,3,4-tetrahydronaphthalen-1-ylcarbamate

To a solution of (1R)—N-{[(3R,8aR)-2-benzyloctahydropyrrolo[1,2-a]pyrazin-3-yl]methyl}1,2,3,4-tetrahydronaphthalen-1-amine (220 mg) in tetrahydrofuran (3 mL) were added saturated aqueous sodium hydrogen carbonate solution (3 mL) and di-tert-butyl bicarbonate (150 mg) under ice-cooling, and the mixture was stirred at room temperature for 60 hr. The reaction mixture was diluted with ethyl acetate (30 mL) and water (30 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (87.0 mg) as a colorless oil.

¹H NMR (300 MHz, CDCl₃): δ 0.98-1.31 (6H, m) 1.33-1.52 (4H, m) 1.51-2.21 (9H, m) 2.29-2.48 (1H, 2.48-3.24 (7H, m) 3.23-3.56 (3H, m) 3.63-3.84 (1H, m) 4.28-5.11 (1H, m) 6.91-7.41 (9H, m).

### (iv) Production of tert-butyl[(3S,8aR)-octahydropyrrolo[1,2-a]pyrazin-3-ylmethyl](1R)-1,2,3,4-tetrahydronaphthalen-1-ylcarbamate

To a solution of tert-butyl{[(3S,8aR)-2-benzyloctahydropyrrolo[1,2-a]pyrazin-3-yl]methyl}(1R)-1,2,3,4-tetrahydronaphthalen-1-ylcarbamate (87.0 mg) in methanol (5.4 mL) was added 20% palladium hydroxide-carbon (51.0 mg, 50% wet), and the mixture was stirred at room temperature for 24 hr under a hydrogen atmosphere (1 atm). The insoluble material was filtered off through a celite pad, and the filtrate was concentrated to give the title compound (62.7 mg) as a brown oil. Without further purification, the compound was used for the next reaction. LC-MS: 386 (MH⁺).

### (v) Production of tert-butyl({(3S,8aR)-2-[(2S)-2-({(2S)-2-[(tert-butoxycarbonyl)(methyl)amino]propanoyl}amino)-2-cyclohexylacetyl]octahydropyrrolo[1,2-a]pyrazin-3-yl}methyl)(1R)-1,2,3,4-tetrahydronaphthalen-1-ylcarbamate

To a solution of tert-butyl[(3S,8aR)-octahydropyrrolo[1,2-a]pyrazin-3-ylmethyl](1R)-1,2,3,4-tetrahydronaphthalen-1-ylcarbamate (62.7 mg) in tetrahydrofuran (1.8 mL) were added (2S)-{[N-(tert-butoxycarbonyl)-N-methyl-L-alanyl]amino}(cyclohexyl)ethanoic acid (62.7 mg) and 4-(4,6-dimethoxy-1,3,5-triazin-2-yl)-4-methylmorpholinium chloride (54.0 mg) under ice-cooling, and the mixture was stirred at room temperature for 4 hr. To the reaction mixture were added (2S)-{[N-(tert-butoxycarbonyl)-N-methyl-L-alanyl]amino}(cyclohexyl)ethanoic acid (18.5 mg) and 4-(4,6-dimethoxy-1,3,5-triazin-2-yl)-4-methylmorpholinium chloride (16.2 mg), and the mixture was stirred again at room temperature for 1 hr. The reaction mixture was diluted with water (30 mL) and ethyl acetate (30 mL), and the aqueous layer was extracted with ethyl acetate (30 mL). The combined organic layers were dried over anhydrous magnesium sulfate, and the insoluble material was filtered off. The filtrate was concentrated under reduced pressure, and the residue was purified by preparative HPLC (acetonitrile/water=20:80→100:0). The fraction containing the title compound was diluted with saturated aqueous sodium hydrogen carbonate solution (100 mL). The aqueous layer was extracted with ethyl acetate (200 mL), the combined organic layers were dried over anhydrous magnesium sulfate, and the insoluble material was filtered off. The filtrate was concentrated under reduced pressure to give the title compound (42.3 mg) as a pale-yellow solid. LC-MS: 710 (MH⁺).

### (vi) Production of N-{(1S)-1-cyclohexyl-2-oxo-2-[(3S,8aR)-3-{[(1R)-1,2,3,4-tetrahydronaphthalen-1-ylamino]methyl}hexahydropyrrolo[1,2-a]pyrazin-2(1H)-yl]ethyl}-N-2-methyl-L-alaninamide trihydrochloride

To a solution of tert-butyl({(3S,8aR)-2-[(2S)-2-({(2S)-2-[(tert-butoxycarbonyl)(methyl)amino]propanoyl}amino)-2-cyclohexylacetyl]octahydropyrrolo[1,2-a]pyrazin-3-yl}methyl)(1R)-1,2,3,4-tetrahydronaphthalen-1-ylcarbamate (42 mg) in ethyl acetate (1 mL) was added 4N hydrogen chloride-ethyl acetate (1 mL), and the mixture was stirred at room temperature for 4 hr. The reaction mixture was diluted with diethyl ether (8 mL), and the mixture was stirred again at room temperature for 15 min. The resulting solid was collected by filtration, and dried under reduced pressure to give the title compound (7.6 mg) as a yellow amorphous powder.

¹H NMR (300 MHz, DMSO-d₆): δ 0.98-1.20 (6H, m) 1.30-1.45 (4H, m) 1.47-1.84 (12H, m) 1.91-2.21 (7H, m) 2.43-2.47 (1H, m) 2.69-2.93 (3H, m) 3.80-3.97 (3H, m) 4.13-4.32 (2H, m) 4.52-4.67 (2H, m) 7.14-7.49 (4H, m) 7.60-8.08 (1H, m) 8.72-9.27 (2H, m) 9.36-9.84 (2H, m) 11.54-12.58 (1H, m).

### Example 16

### Production of N-{(1S)-1-cyclohexyl-2-[(3S,8aR)-3-[4-(naphthalen-1-ylcarbonyl)-1,3-thiazol-2-yl]hexahydropyrrolo[1,2-a]pyrazin-2(1H)-yl]-2-oxoethyl}-N-2-methyl-L-alaninamide dihydrochloride

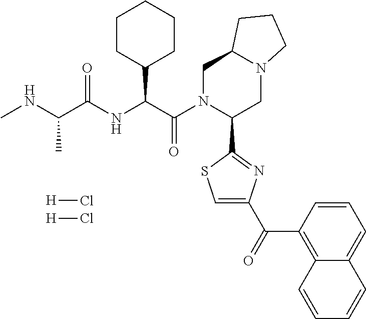

### (i) Production of tert-butyl(3S,8aR)-3-[4-(naphthalen-1-ylcarbonyl)-1,3-thiazol-2-yl]hexahydropyrrolo[1,2-a]pyrazine-2(1H)-carboxylate

To a solution of tert-butyl(3S,8aR)-3-{4-[methoxy(methyl)carbamoyl]-1,3-thiazol-2-yl}hexahydropyrrolo[1,2-a]pyrazine-2(1H)-carboxylate (206 mg) produced in Example 12 (iv) in tetrahydrofuran (5.2 mL) was added 0.5M bromo(naphthalen-1-yl)magnesium tetrahydrofuran solution (3.0 mL) under ice-cooling, and the mixture was allowed to warm to room temperature and stirred for 4 hr. The reaction mixture was diluted with saturated aqueous ammonium chloride solution (40 mL) and ethyl acetate (40 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (194 mg) as a pale-yellow amorphous powder.

¹H NMR (DMSO-d₆, 300 MHz): δ 1.22-1.45 (11H, m), 1.60-1.75 (2H, m), 1.78-1.91 (1H, m), 1.92-2.03 (1H, m), 2.09 (1H, q, J=8.9 Hz), 2.70-3.07 (2H, m), 3.55-3.72 (1H, m), 4.06-4.18 (1H, m), 5.50-5.64 (1H, m), 7.60-7.71 (2H, m), 8.00-8.20 (4H, m), 8.55-8.65 (1H, m), 8.85-9.05 (1H, m).

### (ii) Production of (naphthalen-1-yl){2-[(3S,8aR)-octahydropyrrolo[1,2-a]pyrazin-3-yl]-1,3-thiazol-4-yl}methanone

To a solution of tert-butyl(3S,8aR)-3-[4-(naphthalen-1-ylcarbonyl)-1,3-thiazol-2-yl]hexahydropyrrolo[1,2-a]pyrazine-2(1H)-carboxylate (194 mg) in ethyl acetate (2.1 mL) was added 4N hydrogen chloride-ethyl acetate (2.1 mL), and the mixture was stirred at room temperature for 4 hr. The solvent was evaporated under reduced pressure, and the reaction mixture, was diluted with ethyl acetate (40 mL) and saturated aqueous sodium hydrogen carbonate solution (40 mL). The aqueous layer was extracted with ethyl acetate (40 mL), the combined organic layers were dried over anhydrous magnesium sulfate, and the insoluble material was filtered off. The filtrate was concentrated under reduced pressure, and the residue was subjected to basic silica gel column chromatography (ethyl acetate/hexane=5/95→100/0) to give the title compound (100 mg) as a pale-yellow oil. LC-MS: 364 (MH⁺).

### (iii) Production of tert-butyl[(1S)-2-({(1S)-1-cyclohexyl-2-[(3S,8aR)-3-[4-(naphthalen-1-ylcarbonyl)-1,3-thiazol-2-yl]hexahydropyrrolo[1,2-a]pyrazin-2(1H)-yl]-2-oxoethyl}amino)-1-methyl-2-oxoethyl]methylcarbamate

To a solution of (naphthalen-1-yl){2-[(3S,8aR)-octahydropyrrolo[1,2-a]pyrazin-3-yl]-1,3-thiazol-4-yl}methanone (100 mg) in tetrahydrofuran (2.7 mL) were added (2S)-{[N-(tert-butoxycarbonyl)-N-methyl-L-alanyl]amino}(cyclohexyl)ethanoic acid (132 mg) and 4-(4,6-dimethoxy-1,3,5-triazin-2-yl)-4-methylmorpholinium chloride (114 mg) under ice-cooling, and the mixture was stirred at room temperature for 4 hr. To the reaction mixture were added (2S)-{[N-(tert-butoxycarbonyl)-N-methyl-L-alanyl]amino}(cyclohexyl)ethanoic acid (132 mg) and 4-(4,6-dimethoxy-1,3,5-triazin-2-yl)-4-methylmorpholinium chloride (114 mg), and the mixture was stirred again at room temperature for 12 hr. The reaction mixture was diluted with water (30 mL) and ethyl acetate (30 mL), and the aqueous layer was extracted with ethyl acetate (30 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) and then basic silica gel chromatography (ethyl acetate 100) to give the title compound (81.4 mg) as a colorless oil. LC-MS: 688 (MH⁺).

### (iv) Production of N-{(1S)-1-cyclohexyl-2-[(3S,8aR)-3-[4-(naphthalen-1-ylcarbonyl)-1,3-thiazol-2-yl]hexahydropyrrolo[1,2-a]pyrazin-2(1H)-yl]-2-oxoethyl}-N-2-methyl-L-alaninamide dihydrochloride

To a solution of tert-butyl[(1S)-2-({(1S)-1-cyclohexyl-2-[(3S,8aR)-3-[4-(naphthalen-1-ylcarbonyl)-1,3-thiazol-2-yl]hexahydropyrrolo[1,2-a]pyrazin-2(1H)-yl]-2-oxoethyl}amino)-1-methyl-2-oxoethyl]methylcarbamate (79.0 mg) in ethyl acetate (2.0 mL) was added 4N hydrogen chloride-ethyl acetate (2.0 mL), and the mixture was stirred at room temperature for 1 hr. The reaction mixture was diluted with diethyl ether (10 mL), and the resulting solid was collected by filtration to give the title compound (55.1 mg) as a colorless amorphous powder.

¹H NMR (DMSO-d₆, 300 MHz): δ 0.43-1.30 (7H, m) 1.30-1.88 (11H, m) 1.95-2.36 (3H, m) 3.01-3.22 (1H, m) 3.52-3.75 (2H, m) 3.75-4.40 (4H, m) 4.53-5.00 (2H, m) 5.69-6.60 (1H, m) 7.52-7.81 (2H, m) 7.93-8.26 (4H, m) 8.61-9.46 (5H, m) 10.51-12.34 (1H, m).

### Example 17

### Production of (3S,8aS)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

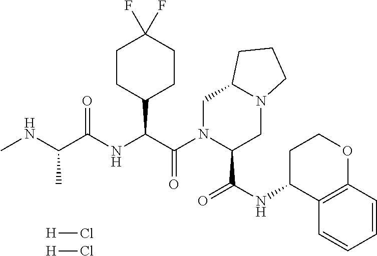

### (i) Production of 2-tert-butyl 3-ethyl(3S,8aS)-hexahydropyrrolo[1,2-a]pyrazine-2,3(1H)-dicarboxylate

To a solution of 1-phenyl-N-[(2S)-pyrrolidin-2-ylmethyl]methanamine (158.4 g) in toluene (1000 mL) were added a solution (500 mL) of ethyl 2,3-dibromopropanoate (195 g) in toluene and triethylamine (267.3 mL), and the mixture was stirred with heating at 90° C. for 18 hr. The reaction mixture was allowed to cool to room temperature, and the insoluble material was filtered off. To the filtrate was added water (500 mL), and the mixture was extracted with ethyl acetate (1000 mL). The organic layer was washed with saturated aqueous sodium hydrogen carbonate solution (500 mL) and saturated brine (500 mL), and dried over anhydrous sodium sulfate, and concentrated to give a pale-yellow oil (224 g). A solution (1000 mL) of this oil in ethanol was added to a suspension (1000 mL) of 10% palladium-carbon (22.4 g) in saturated hydrogen chloride-ethanol, and the mixture was stirred with heating at 90° C., for 18 hr under a hydrogen atmosphere (3 atm). The insoluble material was filtered off through a celite pad, and the filtrate was concentrated to give a pale-yellow oil. This oil was dissolved in saturated aqueous sodium hydrogen carbonate solution (500 mL) and tetrahydrofuran (500 mL), di-tert-butyl bicarbonate (187 g) was added thereto, and the mixture was stirred at room temperature for 18 hr. The mixture was diluted with ethyl acetate (1000 mL), and the organic layer was washed with saturated aqueous sodium hydrogen carbonate solution (500 mL) and saturated brine (500 mL). The organic layer was dried over anhydrous magnesium sulfate, and concentrated under reduced pressure. The residue was purified by silica gel column chromatography (ethyl acetate/hexane=90/10→ethyl acetate) to give the title compound (75.12 g) as a colorless oil. LC-MS: 299 (MH⁺)

¹H NMR (CDCl₃, 400 MHz): δ 1.27 (3H, t, J=7.2 Hz), 1.43 (9H, s), 1.44-1.53 (1H, m), 1.69-1.78 (1H, m), 1.80-1.89 (2H, m), 2.70-2.74 (2H, m), 2.97-3.03 (3H, m), 3.22-3.27 (1H, m), 3.73 (1H, dd, J=13.2, 4.0 Hz), 4.16-4.24 (2H, m), 4.36 (1H, t, J=5.6 Hz).

### (ii) Production of tert-butyl(3S,8aS)-3-[(4R)-3,4-dihydro-2H-Chromen-4-ylcarbamoyl]hexahydropyrrolo[1,2-a]pyrazine-2(1H)-carboxylate

To a solution of 2-tert-butyl 3-ethyl(3S,8aS)-hexahydropyrrolo[1,2-a]pyrazine-2,3(1H)-dicarboxylate (670 mg) in a mixed solvent (5 mL) of tetrahydrofuran/water (4/1) was added lithium hydroxide monohydrate (141 mg), and the mixture was stirred with heating at 50° C. for 3 hr. The reaction mixture was allowed to cool to room temperature, neutralized with 1M hydrochloric acid (3.1 mL), and concentrated under reduced pressure. The obtained residue was dissolved in N,N-dimethylformamide (10 mL), (4R)-3,4-dihydro-2H-chromen-4-amine hydrochloride (500 mg), 1-ethyl-3-(3-dimethylaminopropyl)carbodiimide (1.98 mL), 1-hydroxybenzotriazole (363 mg) and N,N-diisopropylethylamine (0.469 mL) were added thereto, and the reaction mixture was stirred at room temperature for 18 hr. The solvent was evaporated under reduced pressure, and the obtained residue was diluted with ethyl acetate (300 mL) and water (100 mL). The organic layer was dried over anhydrous magnesium sulfate, and the insoluble material was filtered off. The filtrate was concentrated under reduced pressure, the residue was subjected to basic silica gel column chromatography (ethyl acetate/methanol=100/0→80/20), and the collected fractions were concentrated to give the title compound (381 mg) as a colorless amorphous powder. LC-MS: 402.2 (MH⁺)

¹H NMR (DMSO-d₆, 300 MHz): δ 1.26-1.47 (10H, m), 1.48-2.06 (5H, m), 2.55-2.77 (2H, m), 2.82-3.22 (4H, m), 3.48-3.62 (1H, m), 4.19 (3H, t, J=5.4 Hz), 5.02 (1H, q, J=6.9 Hz), 6.70-6.89 (2H, m), 7.06-7.26 (2H, m), 8.31 (1H, d, J=8.1 Hz).

### (iii) Production of (3S,8aS)—N-[(4R)-3,4-dihydro-2H-chromen-4-yl]octahydropyrrolo[1,2-a]pyrazine-3-carboxamide dihydrochloride

tert-Butyl(3S,8aS)-3-[(4R)-3,4-dihydro-2H-chromen-4-ylcarbamoyl]hexahydropyrrolo[1,2-a]pyrazine-2(1H)-carboxylate (381 mg) was dissolved in 4M hydrogen chloride-ethyl acetate solution (2 mL), and the solution was stirred at room temperature for 2 hr. The resulting precipitate was collected by filtration, and dried to give the title compound (355 mg) as a colorless amorphous powder. LC-MS: 302.2 (MH⁺)

### (iv) Production of methyl(2S)-{[(benzyloxy)carbonyl]amino}(4-oxocyclohexyl)ethanoate

To a solution of methyl(2S)-{[(benzyloxy)carbonyl]amino}(1,4-dioxaspiro[4.5]dec-8-yl)ethanoate (1019 mg) in THF (8 mL) was added 1M hydrochloric acid (4 mL), and the mixture was stirred at room temperature for 16 hr. 1M Hydrochloric acid (2 mL) was added thereto, and the mixture was stirred at room temperature for 4 hr. 1M Hydrochloric acid (2 mL) was added thereto again, and the mixture was stirred at room temperature for 4 hr. The mixture was diluted with ethyl acetate, and washed with water. The organic layer was concentrated under reduced pressure, and the residue was purified by silica gel column chromatography (hexane/ethyl acetate=90/10→10/90) to give the title compound (830 mg). LC-MS: 320.2 (MH⁺)

¹H NMR (CDCl₃, 300 MHz): δ 1.47-1.68 (2H, m), 1.86-2.13 (2H, m), 2.19-2.50 (5H, m), 3.77 (3H, s), 4.48 (1H, dd, J=8.9, 5.0 Hz), 5.11 (2H, s), 5.37 (1H, d, J=8.4 Hz), 7.29-7.42 (5H, m).

### (v) Production of methyl(2S)-{[(benzyloxy)carbonyl]amino}(4,4-difluorocyclohexyl)ethanoate

To a solution of methyl(2S)-{[(benzyloxy)carbonyl]amino}(4-oxocyclohexyl)ethanoate (830 mg) in dichloromethane (5 mL) was added bis(2-methoxyethyl)aminosulfur trifluoride (1.198 mL) with stirring under ice-cooling, and the mixture was stirred at room temperature for 18 hr. The mixture was poured into 5% aqueous sodium bicarbonate, and the organic solvent was evaporated under reduced pressure. The mixture was extracted with ethyl acetate. The organic layer was concentrated under reduced pressure, the residue was subjected to silica gel column chromatography (hexane/ethyl acetate=95/5→50/50), and the object fractions were concentrated under reduced pressure. The residue was dissolved in a mixed solvent of acetonitrile/water (4 mL/4 mL), ruthenium (III) chloride (16 mg) and sodium periodate (556 mg) were successively added thereto, and the mixture was stirred at room temperature for 6 hr. To the mixture was added 10% aqueous sodium thiosulfate solution, and the mixture was extracted with ethyl acetate. The organic layer was concentrated under reduced pressure, and the residue was purified by basic silica gel column chromatography (hexane/ethyl acetate=90/10→50/50) to give the title compound (448 mg). LC-MS: 342.1 (MH⁺)

¹H NMR (CDCl₃, 300 MHz): δ 1.34-2.21 (9H, m), 3.76 (3H, s), 4.40 (1H, dd, J=9.0, 5.1 Hz), 5.11 (2H, s), 5.32 (1H, d, J=8.7 Hz), 7.29-7.42 (5H, m).

### (vi) Production of (2S)-{[(benzyloxy)carbonyl]amino}(4,4-difluorocyclohexyl)ethanoic acid

To a solution of methyl(2S)-{[(benzyloxy)carbonyl]amino}(4,4-difluorocyclohexyl)ethanoate (446 mg) in a mixed solvent of THF (8 mL)/water (3 mL) was added lithium hydroxide hydrate (219 mg), and the mixture was stirred at room temperature for 18 hr. 1M Hydrochloric acid (7.83 mL) was added thereto under ice-cooling, and the mixture was extracted with ethyl acetate. The organic layer was washed with water, dried over magnesium sulfate, and filtered, and the filtrate was concentrated under reduced pressure. To the residue was added diisopropyl ether, and the precipitate was collected by filtration, washed with diisopropyl ether, and dried under reduced pressure to give the title compound (326 mg).

¹H NMR (CDCl₃, 300 MHz): δ 1.37-2.24 (9H, m), 4.45 (1H, dd, J=8.7, 4.8 Hz), 5.12 (2H, s), 5.29 (1H, d, J=8.7 Hz), 7.28-7.42 (5H, m).

### (vii) Production of (3S,8aS)-2-[(2S)-2-amino-2-(4,4-difluorocyclohexyl)acetyl]-N-[(4R)-3,4-dihydro-2H-chromen-4-yl]octahydropyrrolo[1,2-a]pyrazine-3-carboxamide

To a solution of (3S,8aS)—N-[(4R)-3,4-dihydro-2H-chromen-4-yl]octahydropyrrolo[1,2-a]pyrazine-3-carboxamide dihydrochloride (200 mg), (2S)-{[(benzyloxy)carbonyl]amino}(4,4-difluorocyclohexyl)ethanoic acid (192 mg) and N,N-diisopropylethylamine (0.186 mL) in N,N-dimethylformamide (3 mL) was added O-(7-azabenzotriazol-1-yl)-N,N,N′,N′-tetramethyluronium hexafluorophosphate (406 mg), and the reaction mixture was stirred at room temperature for 2 hr. The mixture was diluted with ethyl acetate (250 mL), and washed with water (50 mL). The organic layer was washed with saturated brine (50 mL), and dried over anhydrous magnesium sulfate, and the insoluble material was filtered off. The filtrate was concentrated, and the residue was dissolved in methanol. 20% Palladium hydroxide-carbon (35 mg) was added thereto, and the mixture was stirred at room temperature for 5 hr under a hydrogen atmosphere (3 atm). The insoluble material was filtered off through a celite pad, and the filtrate was concentrated to give the title compound (820 mg) as a crude product. Without further purification, the compound was used for the next reaction. LC-MS: 477.3 (MH⁺).

### (viii) Production of (3S,8aS)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

To a solution of the crude product (820 mg) of (3S,8aS)-2-[(2S)-2-amino-2-(4,4-difluorocyclohexyl)acetyl]-N-[(4R)-3,4-dihydro-2H-chromen-4-yl]octahydropyrrolo[1,2-a]pyrazine-3-carboxamide, N-(tert-butoxycarbonyl)-N-methyl-L-alanine (109 mg) and N,N-diisopropylethylamine (0.186 mL) in N,N-dimethylformamide (3 mL) was added O-(7-azabenzotriazol-1-yl)-N,N,N′,N′-tetramethyluronium hexafluorophosphate (305 mg), and the reaction mixture was stirred at room temperature for 1 hr. The mixture was diluted with ethyl acetate (250 mL), and washed with water (100 mL). The organic layer was washed with saturated brine, and dried over anhydrous magnesium sulfate, and the insoluble material was filtered off, and the filtrate was concentrated. The residue was subjected to silica gel column chromatography (ethyl acetate/hexane=30/70→100/0→ethyl acetate/methanol=90/10), and the collected fractions were concentrated to give a colorless amorphous powder. This amorphous powder was dissolved in 4M hydrogen chloride-ethyl acetate solution (3 mL), and the solution was stirred at room temperature for 1 hr. To this solution was added diethyl ether (10 mL), and the precipitate was collected by filtration, and dried under reduced pressure to give the title compound (17 mg) as a colorless amorphous powder. LC-MS: 562.4 (MH⁺)

¹H NMR (DMSO-d₆, 300 MHz): δ 1.28-1.49 (5H, m), 1.61-2.31 (14H, m), 2.43-2.48 (3H, m), 3.07 (1H, d, J=8.5 Hz), 3.47-3.73 (3H, m), 3.78-4.30 (5H, m), 4.67 (1H, t, J=5.3 Hz), 4.80 (1H, t, J=7.2 Hz), 4.93-5.04 (1H, m), 6.75-6.95 (2H, m), 7.16 (2H, t, J=6.9 Hz), 8.65-9.04 (3H, m), 9.19-9.47 (1H, m), 10.88-11.28 (1H, m).

### Example 18

### Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

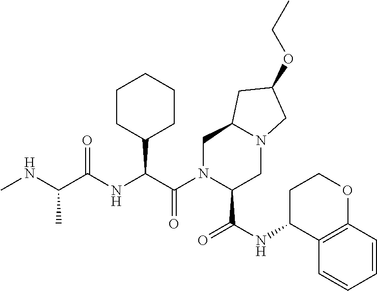

### (i) Production of (4R)-1-(tert-butoxycarbonyl)-4-ethoxy-D-praline

To a mixture of (4R)-1-(tert-butoxycarbonyl)-4-hydroxy-D-proline (30.0 g) in tetrahydrofuran (300 mL) was added 60% sodium hydride (13.0 g) under ice-cooling, and the mixture was stirred at 0° C. for 30 min. To the reaction mixture was added ethyl iodide (51.9 mL), and the mixture was allowed to warm to room temperature and stirred for 18 hr. To the mixture were added water (150 mL), 1M hydrochloric acid (150 mL) and sodium chloride (about 40 g), and the mixture was extracted with ethyl acetate (500 mL). The organic layer was dried over anhydrous magnesium sulfate, and the insoluble material was filtered off. The filtrate was concentrated under reduced pressure, the residue was subjected to silica gel column chromatography (ethyl acetate), and the collected fractions were concentrated. The obtained oil was crystallized from diethyl ether (50 mL)/hexane (200 mL) to give the title compound (31.6 g) as colorless crystals.

¹H NMR (DMSO-d₆, 300 MHz): δ 0.99-1.10 (3H, m), 1.30-1.44 (9H, m), 1.87-2.02 (1H, m), 2.22-2.45 (1H, m), 3.10-3.21 (1H, m), 3.26-3.44 (2H, m), 3.47-3.61 (1H, m), 3.94-4.05 (1H, m), 4.08-4.18 (1H, m), 12.43 (1H, brs).

### (ii) Production of tert-butyl(2R,4R)-2-(benzylcarbamoyl)-4-ethoxypyrrolidine-1-carboxylate

To a suspension (300 mL) of (4R)-1-(tert-butoxycarbonyl)-4-ethoxy-D-proline (26.0 g), benzylamine (13.0 mL) and 1-hydroxybenzotriazole (16.05 g) in acetonitrile was added 1-ethyl-3-(3-dimethylaminopropyl)carbodiimide hydrochloride (31.06 g) under ice-cooling, and the mixture was allowed to warm to room temperature and stirred for 2 hr. To this reaction mixture was added water (100 mL), and the mixture was extracted with ethyl acetate (500 mL). The organic layer was washed with saturated aqueous sodium hydrogen carbonate solution (150 mL×2) and saturated brine (100 mL), dried over anhydrous magnesium sulfate, and concentrated under reduced pressure. The residue was crystallized from ether (10 mL)/hexane (50 mL) to give the title compound (36.0 g) as colorless crystals. LC-MS: 349.2 (MH⁺)

¹H NMR (DMSO-d₆, 300 MHz): δ 1.04 (3H, t, J=6.9 Hz), 1.23-1.45 (9H, m), 1.79-2.00 (1H, m), 2.22-2.44 (1H, m), 3.18-3.44 (3H, m), 3.53-3.66 (1H, m), 3.85-4.43 (4H, m), 7.15-7.35 (5H, m), 8.01-8.27 (1H, m).

### (iii) Production of N-benzyl-1-[(2R,4R)-4-ethoxypyrrolidin-2-yl]methanamine

To a solution of tert-butyl(2R,4R)-2-(benzylcarbamoyl)-4-ethoxypyrrolidine-1-carboxylate (35.4 g) in a mixed solvent of ethyl acetate (100 mL)/methanol (25 mL) was added 4M hydrogen chloride-ethyl acetate solution (125 mL), and the mixture was stirred at room temperature for 18 hr, and concentrated. The residue was filtered off through a pad filled with Amberlyst A21 (150 g, registered trade mark), the pad was washed with methanol (500 mL), and the filtrate was concentrated. The residue was dissolved in tetrahydrofuran (100 mL), and the solution was added to a suspension (300 mL) of lithium aluminum hydride (7.71 g) in tetrahydrofuran under ice-cooling. The reaction mixture was heated under reflux for hr. The reaction mixture was cooled to 0° C., and sodium sulfate decahydrate (15 g) was added thereto. The insoluble material was filtered off, and the filtrate was concentrated under reduced pressure. Without further purification, the compound was used for the next reaction. LC-MS: 235.2 (MH⁺)

### (iv-a) Production of 2-tert-butyl 3-methyl(3S,7R,8aR)-7-ethoxyhexahydropyrrolo[1,2-a]pyrazine-2,3(1H)-dicarboxylate

To a solution of N-benzyl-1-[(2R,4R)-4-ethoxypyrrolidin-2-yl]methanamine (16.1 g) in toluene (150 mL) was added a solution of methyl 2,3-dibromopropanoate (14.4 g) in toluene (100 mL). To this mixture was added triethylamine (22.1 mL), and the mixture was stirred at 90° C. for 12 hr. The reaction mixture was allowed to cool to room temperature, and diluted with ethyl acetate (250 mL) and water (200 mL). The organic layer was dried over anhydrous magnesium sulfate, the insoluble material was filtered off, and the filtrate was concentrated under reduced pressure. The obtained residue (18.8 g) was dissolved in 5-10% hydrogen chloride-methanol solution (200 mL), 10% palladium carbon (1.88 g) was added to the solution, and the mixture was stirred at room temperature for 3 hr under a hydrogen atmosphere (3 atm). The insoluble material was filtered off through a celite pad, and the filtrate was concentrated to give a pale-yellow oil (21 g). This oil (21 g) was dissolved in saturated aqueous sodium hydrogen carbonate solution (125 mL) and tetrahydrofuran (125 mL), di-tert-butyl bicarbonate (13.4 g) was added to the resulting two phase-mixture, and the mixture was stirred at room temperature for 2 hr. The mixture was diluted with ethyl acetate (300 mL), and washed with saturated brine (100 mL). The organic layer was dried over anhydrous magnesium sulfate, the insoluble material was filtered off, and the filtrate was concentrated. The residue was purified by silica gel column chromatography (hexane→ethyl acetate→ethyl acetate/methanol=80/20) to give the title compound (5.42 g) as a colorless oil. LC-MS: 329.2 (MH⁺)

¹H NMR (CDCl₃, 300 MHz): δ 1.19 (3H, t, J=7.0 Hz), 1.33-1.58 (10H, m), 1.88 (1H, brs), 2.12-2.30 (3H, m), 2.78-3.02 (1H, m), 3.05-3.14 (1H, m), 3.36-3.46 (2H, m), 3.46-3.59 (1H, m), 3.70-3.77 (3H, m), 3.88-4.13 (2H, m), 4.52-4.87 (1H, m).

### (iv-b) Production of 2-tert-butyl 3-methyl(3S,7R,8aR)-7-ethoxyhexahydropyrrolo[1,2-a]pyrazine-2,3(1H)-dicarboxylate and 2-tert-butyl 3-methyl(3R,7R,8aR)-7-ethoxyhexahydropyrrolo[1,2-a]pyrazine-2,3(1H)-dicarboxylate

To a solution of N-benzyl-1-[(2R,4R)-4-ethoxypyrrolidin-2-yl]methanamine (19.8 g) in toluene (158 mL) was added methyl 2,3-dibromopropanoate (9.1 mL). To this mixture was added triethylamine (28.3 mL), and the mixture was stirred at 80° C. for 5 hr. The reaction mixture was allowed to cool to room temperature, water was added thereto, and the mixture was extracted with ethyl acetate. The extract was dried over anhydrous magnesium sulfate, and filtered, and the filtrate was concentrated under reduced pressure. The insoluble material was filtered off through a silica gel pad (100 g), and washed with ethyl acetate, and the filtrate was concentrated under reduced pressure. The obtained residue was dissolved in 5-10% hydrogen chloride-methanol solution (200 mL), 10% palladium carbon (4.9 g) was added thereto, and the mixture was stirred at room temperature for 2 hr under a hydrogen atmosphere (3 atm). The insoluble material was filtered off through a celite pad, and washed with methanol. The filtrate was concentrated to give a pale-yellow oil. To the obtained pale-yellow oil were added tetrahydrofuran (220 mL) and saturated aqueous sodium hydrogen carbonate solution (220 mL), and then di-tert-butyl bicarbonate (16.8 g) was added to the resulting two-phase mixture, and the mixture was stirred at room temperature for 3 hr. Water was added to the reaction mixture, and the mixture was extracted with ethyl acetate. The extract was washed with saturated brine, and dried over anhydrous magnesium sulfate. The insoluble material was filtered off, and the filtrate was concentrated. The residue was purified by silica gel column chromatography (hexane/ethyl acetate/methanol=90/10/0→0/100/0→0/85/15) to give 2-tert-butyl 3-methyl(3S,7R,8aR)-7-ethoxyhexahydropyrrolo[1,2-a]pyrazine-2,3(1H)-dicarboxylate (6.9 g) and 2-tert-butyl 3-methyl(3R,7R,8aR)-7-ethoxyhexahydropyrrolo[1,2-a]pyrazine-2,3(1H)-dicarboxylate (10.6 g) as a colorless oil.

### 2-tert-butyl 3-methyl(3S,7R,8aR)-7-ethoxyhexahydropyrrolo[1,2-a]pyrazine-2,3(1H)-dicarboxylate

¹H NMR (DMSO-d₆, 300 MHz): δ 1.07 (3H, t, J=7.1 Hz), 1.14-1.27 (1H, m), 1.33-1.43 (9H, m), 1.72-1.88 (1H, m), 2.06-2.27 (3H, m), 2.58-2.85 (1H, m), 2.91-2.97 (1H, m), 3.25-3.41 (3H, m), 3.65-3.70 (3H, m), 3.81-3.96 (2H, m), 4.60 (1H, dd, J=25.2, 3.3 Hz).

### 2-tert-butyl 3-methyl(3R,7R,8aR)-7-ethoxyhexahydropyrrolo[1,2-a]pyrazine-2,3(1H)-dicarboxylate

¹H NMR (DMSO-d₆, 300 MHz): δ 1.08 (3H, t, J=7.1 Hz), 1.38-1.46 (10H, m), 2.08-2.21 (1H, m), 2.64-2.86 (4H, m), 3.03-3.21 (2H, m), 3.26-3.42 (2H, m), 3.57-3.66 (1H, m), 3.64 (3H, s), 3.87-3.98 (1H, m), 4.25-4.31 (1H, m).

### (iv-c) Production of 2-tert-butyl 3-methyl(3S,7R,8aR)-7-ethoxyhexahydropyrrolo[1,2-a]pyrazine-2,3(1H)-dicarboxylate

To a solution of 2-tert-butyl 3-methyl(3R,7R,8aR)-7-ethoxyhexahydropyrrolo[1,2-a]pyrazine-2,3(1H)-dicarboxylate (10.6 g) in methanol (42.4 mL) was added 28% sodium methoxide (1.25 g), and the mixture was stirred at 60° C. for 5 hr. the mixture was allowed to cool to room temperature, and concentrated under reduced pressure. The obtained residue was purified by silica gel column chromatography (hexane/methyl acetate=80/20→20/80), and the object fractions were collected, and concentrated to give the title compound (9.56 g).

¹H NMR (DMSO-d₆, 300 MHz): δ 1.07 (3H, t, J=7.1 Hz), 1.14-1.27 (1H, m), 1.33-1.43 (9H, m), 1.72-1.88 (1H, m), 2.06-2.27 (3H, m), 2.58-2.85 (1H, m), 2.91-2.97 (1H, m), 3.25-3.41 (3H, m), 3.65-3.70 (3H, m), 3.81-3.96 (2H, m), 4.60 (1H, dd, J=25.2, 3.3 Hz).

### (v) Production of tert-butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate

To a solution of 2-tert-butyl 3-methyl(3S,7R,8aR)-7-ethoxyhexahydropyrrolo[1,2-a]pyrazine-2,3(1H)-dicarboxylate (3.6 g) in tetrahydrofuran (90 mL)/water (18 mL) was added lithium hydroxide monohydrate (736 mg) at room temperature, and the reaction mixture was stirred at 50° C. for 5 hr. The reaction mixture was allowed to cool to room temperature, neutralized with 1M hydrochloric acid (17.5 mL), and concentrated under reduced pressure. The residue was subjected twice to azeotropic distillation with toluene, and the residue was dried in vacuum for 5 hr. The obtained residue was dissolved in N,N-dimethylformamide (54 mL), and 1-hydroxybenzotriazole (1.78 g) and N,N-diisopropylethylamine (5.73 mL) were added thereto. The 1-ethyl-3-(3-dimethylaminopropyl)carbodiimide hydrochloride (6.30 g) was added thereto at 0° C., and the mixture was stirred at room temperature for 30 min. (4R)-3,4-Dihydro-2H-chromen-4-amine hydrochloride (2.14 g) was added thereto, and the reaction mixture was stirred at room temperature for 3 days. The mixture was poured into ethyl acetate/water, and the organic layer was separated. The organic layer was washed with saturated aqueous sodium hydrogen carbonate and saturated brine, dried over anhydrous magnesium sulfate, and filtered. The filtrate was concentrated under reduced pressure, and the residue was purified by silica gel column chromatography (hexane/ethyl acetate=50/50→20/80). The object fractions were collected, and concentrated under reduced pressure, and the residue was dried in vacuum for 1 hr to give the title compound (4.04 g) as a white powder.

LC-MS: 446.3 (MH⁺)

¹H NMR (DMSO-d₆, 300 MHz): δ 1.09 (3H, t, J=7.1 Hz), 1.15-1.35 (1H, m), 1.35-1.45 (9H, m), 1.65-2.25 (6H, m), 2.87-3.08 (2H, m), 3.33 (2H, q, J=7.1 Hz), 3.30-3.45 (1H, m), 3.80-4.00 (2H, m), 4.15-4.35 (2H, m), 4.37-4.53 (1H, m), 5.02-5.14 (1H, m), 6.73-6.89 (2H, m), 7.10-7.19 (2H, m), 8.20-8.40 (1H, m).

### (vi) Production of (3S,7R,8aR)—N-[(4R)-3,4-dihydro-2H-chromen-4-yl]-7-ethoxyoctahydropyrrolo[1,2-a]pyrazine-3-carboxamide dihydrochloride

To a solution of tert-butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate (2.0 g) in methanol (5 mL) was added 4M hydrogen chloride-cyclopentyl methyl ether solution (15 mL), and the mixture was stirred at room temperature for 1 hr, and concentrated to give the title compound as a crude product (1.9 g). Without further purification, the compound was used for the next reaction. LC-MS: 346.2 (MH⁺)

### (vii) Production of tert-butyl{(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate

A solution of (3S,7R,8aR)—N-[(4R)-3,4-dihydro-2H-chromen-4-yl]-7-ethoxyoctahydropyrrolo[1,2-a]pyrazine-3-carboxamide dihydrochloride (1.9 g), N,N-diisopropylethylamine (2.35 mL), and (2S)-[(tert-butoxycarbonyl)amino](cyclohexyl)ethanoic acid (1.39 g) in N,N-dimethylformamide was cooled to 0° C., O-(7-azabenzotriazol-1-yl)-N,N,N′,N′-tetramethyluronium hexafluorophosphate (2.56 g) was added thereto, and the mixture was allowed to warm to room temperature, and stirred for 3 hr. To the reaction mixture was added water (50 mL), and the mixture was diluted with ethyl acetate (350 mL). The organic layer was dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (2.6 g) as a colorless oil. LC-MS: 585.4 (MH⁺)

¹H NMR (CDCl₃, 300 MHz): δ 0.88-1.40 (17H, m), 1.46-2.28 (11H, m), 2.97 (1H, t, J=8.9 Hz), 3.14-3.74 (6H, m), 3.88-4.54 (5H, m), 4.69-5.14 (2H, m), 6.69-6.91 (3H, m), 7.06-7.28 (2H, m), 8.06-8.22 (1H, m).

### (viii) Production of tert-butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate

To a solution of tert-butyl{(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (2.6 g) in methanol (5 mL) was added 4M hydrogen chloride-cyclopentyl methyl ether solution (15 mL), and the mixture was stirred at room temperature for 1 hr, and concentrated. To a suspension of this residue, N,N-diisopropylethylamine (2.34 mL), N-(tert-butoxycarbonyl)-N-methyl-L-alanine (1.09 g) and 1-hydroxybenzotriazole (667 mg) in tetrahydrofuran (15 mL) was added 1-ethyl-3-(3-dimethylaminopropyl)carbodiimide hydrochloride (1.72 g), and the mixture was stirred at room temperature for 12 hr. To this reaction mixture was added water (50 mL), and the mixture was extracted with ethyl acetate (350 mL). The organic layer was washed with saturated aqueous sodium hydrogen carbonate solution (50 mL) and saturated brine (100 mL), and dried over anhydrous magnesium sulfate, and the insoluble material was filtered off. The filtrate was concentrated under reduced pressure. The residue was purified by silica gel column chromatography (ethyl acetate/hexane=10/90→100/0) to give the title compound (2.23 g) as a colorless amorphous solid. LC-MS: 670.4 (MH⁺)

¹H NMR (CDCl₃, 300 MHz): δ 0.81-1.34 (15H, m), 1.44-1.80 (14H, m), 1.86-2.39 (6H, m), 2.73-2.82 (3H, m), 3.04-3.72 (3H, m), 3.32-3.45 (2H, m), 3.82-4.33 (2H, m), 4.41-5.38 (4H, m), 6.61-7.87 (6H, m).

### (ix) Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

To a solution of tert-butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (2.20 g) in ethyl acetate (5 mL) was added 4M hydrogen chloride-cyclopentyl methyl ether solution (15 mL), and the reaction mixture was stirred at room temperature for 1 hr. To the mixture was added 1N aqueous sodium hydroxide solution (70 mL) at room temperature, and the mixture was extracted with ethyl acetate (350 mL×2). The combined organic layers were dried over anhydrous magnesium sulfate, and the insoluble material was filtered off. The filtrate was concentrated under reduced pressure. The residue was crystallized from hexane (35 mL)/ethyl acetate (10 mL) to give the title compound (882 mg) as colorless crystals. The mother liquor was concentrated, and the residue was crystallized from ether (20 mL)/hexane (5 mL) to give the title compound (434 mg) as colorless crystals. LC-MS: 570.4 (MH⁺)

¹H NMR (DMSO-d₆, 300 MHz): δ 0.80-1.34 (12H, m), 1.51-1.83 (7H, m), 1.87-2.26 (9H, m), 2.88-3.03 (2H, m), 3.24-3.40 (3H, m), 3.41-3.66 (1H, m), 3.90-4.08 (2H, m), 4.12-4.26 (2H, m), 4.34-5.16 (3H, m), 6.72-6.80 (1H, m), 6.80-6.92 (1H, m), 7.08-7.28 (2H, m), 7.83-8.02 (1H, m), 8.12-8.41 (1H, m).

### Example 19

### Production of (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide dihydrochloride

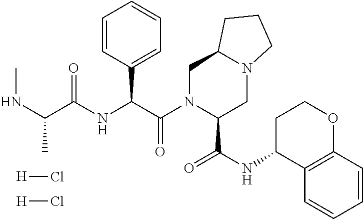

### (i) Production of benzyl{(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-phenylethyl}carbamate

tert-Butyl(3S,8aR)-3-[(4R)-3,4-dihydro-2H-chromen-4-ylcarbamoyl]hexahydropyrrolo[1,2-a]pyrazine-2(1H)-carboxylate (450 mg) was dissolved in ethyl acetate (3 mL), 4M hydrogen chloride-ethyl acetate solution (3 mL) was added thereto, and the mixture was stirred at room temperature for 1 hr. The mixture was concentrated under reduced pressure, and the residue was collected by filtration, washed with ether, and dried under reduced pressure to give a colorless amorphous powder (410 mg). The obtained colorless amorphous powder (200 mg), (2S)-{[(benzyloxy)carbonyl]amino}(phenyl)ethanoic acid (230 mg), O-(7-azabenzotriazol-1-yl)-N,N,N′,N′-tetramethyluronium hexafluorophosphate (406 mg) and N,N-diisopropylethylamine (0.558 mL) were mixed in N,N-dimethylformamide (5 mL), and the mixture was stirred at room temperature for 18 hr. The mixture was diluted with ethyl acetate (30 mL), and washed with water (30 mL), saturated aqueous sodium hydrogen carbonate solution (50 mL) and saturated brine (50 mL). The organic layer was dried over anhydrous magnesium sulfate, and concentrated under reduced pressure. The residue was purified by silica gel column chromatography (ethyl acetate/hexane=10/90→80/20) to give the title compound (220 mg) as a pale-yellow amorphous powder. LC-MS: 569.3 (MH⁺)

¹H NMR (DMSO-d₆, 300 MHz): δ 0.93-1.36 (2H, m), 1.42-2.30 (6H, m), 2.69 (2H, s), 2.90 (1H, m), 3.37-3.63 (1H, m), 3.80-4.55 (3H, m), 4.80-5.16 (4H, m), 5.45-5.85 (1H, m), 6.68-6.95 (2H, m), 7.02-7.19 (2H, m), 7.19-7.52 (10H, m), 7.71-8.36 (2H, m).

### (ii) Production of tert-butyl[(1S)-2-({(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-phenylethyl}amino)-1-methyl-2-oxoethyl]methylcarbamate

To a solution of benzyl{(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-phenylethyl}carbamate (220 mg) in methanol (10 mL) was added 10% palladium-carbon (30 mg, 50% water-containing product), and the mixture was stirred at room temperature for 3 hr under a hydrogen atmosphere. The insoluble material was filtered off through a celite pad, and the filtrate was concentrated to give (3S,8aR)-2-[(2S)-2-amino-2-phenylacetyl]-N-[(4R)-3,4-dihydro-2H-chromen-4-yl]octahydropyrrolo[1,2-a]pyrazine-3-carboxamide as a colorless oil. The obtained (3S,8aR)-2-[(2S)-2-amino-2-phenylacetyl]-N-[(4R)-3,4-dihydro-2H-chromen-4-yl]octahydropyrrolo[1,2-a]pyrazine-3-carboxamide, N-(tert-butoxycarbonyl)-N-methyl-L-alanine (118 mg), O-(7-azabenzotriazol-1-yl)-N,N,N′,N′-tetramethyluronium hexafluorophosphate (295 mg) and N,N-diisopropylethylamine (0.405 mL) were mixed in N,N-dimethylformamide (5 mL), and the mixture was stirred at room temperature for 18 hr. The mixture was diluted with ethyl acetate (30 mL), and washed with water (30 mL) and saturated brine (50 mL). The organic layer was dried over anhydrous magnesium sulfate, and concentrated under reduced pressure. The residue was purified by silica gel column chromatography (ethyl acetate/hexane=10/90→80/20) to give the title compound (200 mg) as a colorless amorphous powder. LC-MS: 620.4 (MH⁺)

### (iii) Production of (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide dihydrochloride

tert-Butyl[(1S)-2-({(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-phenylethyl}amino)-1-methyl-2-oxoethyl]methylcarbamate (200 mg) was dissolved in ethyl acetate (3 mL), 4M hydrogen chloride-ethyl acetate solution (3 mL) was added thereto, and the mixture was stirred at room temperature for 2 hr. The mixture was concentrated under reduced pressure, and the residue was collected by filtration, washed with ether, and dried under reduced pressure to give the title compound (150 mg) as a white amorphous powder. LC-MS: 520.3 (MH⁺)

¹H NMR (DMSO-d₆, 300 MHz): δ 1.22-2.14 (10H, m), 2.41 (3H, brs), 2.64-3.29 (3H, m), 3.41-3.97 (4H, m), 4.05-4.76 (4H, m), 4.89-6.26 (2H, m), 6.67-6.99 (2H, m), 7.06-7.64 (7H, m), 8.70-9.61 (3H, m), 12.04 (1H, brs).

### Example 20

### Production of (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide

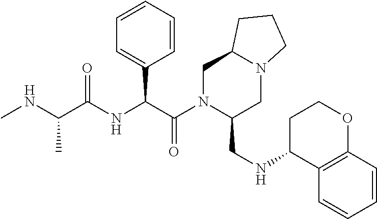

To (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide dihydrochloride (185 mg) were added water (20 mL) and saturated aqueous sodium hydrogen carbonate solution (20 mL), and the mixture was extracted with ethyl acetate (40 mL×2). The combined organic layers were dried over anhydrous magnesium sulfate, and the insoluble material was filtered off. The filtrate was concentrated under reduced pressure to give the title compound (127 mg) as a colorless amorphous powder. MS: 520.3 (MH⁺)

¹H NMR (DMSO-d₆, 300 MHz): δ 1.23-1.46 (3H, m), 1.79-2.08 (4H, m), 2.32-2.46 (4H, m), 2.59-2.94 (1H, m), 3.31-3.62 (2H, m), 3.80-3.95 (1H, m), 4.02-4.29 (3H, m), 4.48-5.26 (5H, m), 5.68-6.19 (1H, m), 6.71-6.97 (2H, m), 7.11-7.25 (2H, m), 7.28-7.56 (5H, m), 8.26-8.64 (1H, m), 8.74-8.97 (1H, m), 9.13-9.33 (1H, m), 9.35-9.66 (1H, m).

### Example 21

### Production of (3S,7R,8aR)—N-[(4R)-3,4-dihydro-2H-chromen-4-yl]-7-ethoxy-2-{(2S)-2-[(N-methyl-L-alanyl)amino]-2-phenylacetyl}octahydropyrrolo[1,2-a]pyrazine-3-carboxamide

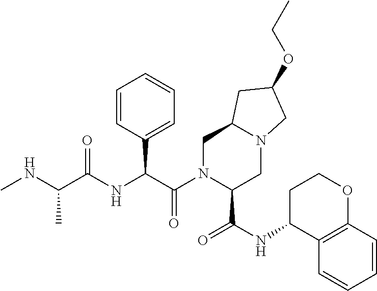

### (i) Production of tert-butyl{(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxo-1-phenylethyl}carbamate

To a solution of (3S,7R,8aR)—N-[(4R)-3,4-dihydro-2H-chromen-4-yl]-7-ethoxyoctahydropyrrolo[1,2-a]pyrazine-3-carboxamide dihydrochloride (367 mg) in N,N-dimethylformamide (8.8 mL) were added N,N-diisopropylethylamine (0.92 mL), (2S)-[(tert-butoxycarbonyl)amino](phenyl)ethanoic acid (332 mg) and O-(7-azabenzotriazol-1-yl)-N,N,N′,N′-tetramethyluronium hexafluorophosphate (670 mg), and the mixture was stirred at room temperature for 18 hr. The reaction mixture was diluted with ethyl acetate (100 mL) and saturated aqueous sodium hydrogen carbonate solution (100 mL), and the aqueous layer was extracted with ethyl acetate (100 mL). The organic layer was dried over anhydrous magnesium sulfate, and the insoluble material was filtered off. The filtrate was concentrated under reduced pressure, and the residue was purified by silica gel column chromatography (ethyl acetate/hexane=10/90→480/20) to give the title compound (373 mg) as a yellow oil. LC-MS: 579.4 (MH⁺)

¹H NMR (DMSO-d₆, 300 MHz): δ 1.07 (3H, t, J=7.0 Hz), 1.21-1.63 (11H, m), 1.81-2.29 (5H, m), 2.90-2.99 (1H, m), 3.27-3.31 (2H, m), 3.33-3.37 (1H, m), 3.40-3.59 (1H, m), 3.76-4.02 (2H, m), 4.13-4.29 (2H, m), 4.37-5.00 (1H, m), 5.00-5.23 (1H, m), 5.39-5.84 (1H, m), 6.68-6.81 (1H, m), 6.82-6.97 (1H, s), 7.03-7.61 (8H, m), 7.98-8.45 (1H, m).

### (ii) Production of (3S,7R,8aR)-2-[(2S)-2-amino-2-phenylacetyl]-N-[(4R)-3,4-dihydro-2H-chromen-4-yl]-7-ethoxyoctahydropyrrolo[1,2-a]pyrazine-3-carboxamide dihydrochloride

To a solution of tert-butyl{(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxo-1-phenylethyl}carbamate (373 mg) in ethyl acetate (3.3 mL) was added 4M hydrogen chloride-ethyl acetate solution (6.5 mL), and the mixture was stirred at room temperature for 30 min. The reaction mixture was diluted with diethyl ether (20 mL), and the resulting solid was collected by filtration, and washed with diethyl ether (10 mL) to give the title compound (283 mg) as a flesh-colored solid. LC-MS: 479.3 (MH⁺)

¹H NMR (DMSO-d₆, 300 MHz): δ 0.97-1.12 (3H, m), 1.71-1.90 (2H, m), 1.96-2.07 (1H, m), 2.18-2.29 (1H, m), 3.09-3.23 (1H, m), 3.27-3.55 (4H, m), 3.73-3.85 (1H, m), 3.97-4.43 (8H, m), 4.68-5.05 (2H, m), 5.61-5.95 (1H, m), 6.79 (1H, d, J=7.9 Hz), 6.86-6.98 (1H, m), 7.11-7.29 (2H, m), 7.42-7.69 (4H, m), 8.73 (2H, br s), 9.11 (1H, d, J=7.9 Hz), 12.45-12.69 (1H, m).

### (iii) Production of tert-butyl[(1S)-2-({(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxo-1-phenylethyl}amino)-1-methyl-2-oxoethyl]methylcarbamate

A solution of N-(tert-butoxycarbonyl)-N-methyl-L-alanine (155 mg) in N,N-dimethylformamide (3 mL) was cooled to 0° C., 1-hydroxybenzotriazole (76 mg) and N-[3-(dimethylamino)propyl]-N′-ethylcarbodiimide hydrochloride (195 mg) were added thereto, and the reaction mixture was allowed to warm to room temperature and stirred at the same temperature for 30 min. A solution of (3S,7R,8aR)-2-[(2S)-2-amino-2-phenylacetyl]-N-[(4R)-3,4-dihydro-2H-chromen-4-yl]-7-ethoxyoctahydropyrrolo[1,2-a]pyrazine-3-carboxamide (280 mg) and N,N-diisopropylethylamine (0.54 mL) in N,N-dimethylformamide (2 mL) was added thereto, and the mixture was stirred at room temperature for 18 hr. The reaction mixture was diluted with ethyl acetate (100 mL) and saturated aqueous sodium hydrogen carbonate solution (50 mL), and the aqueous layer was extracted with ethyl acetate (100 mL). The organic layer was dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (164 mg) as a colorless amorphous powder. LC-MS 664.4 (MH⁺)

¹H NMR (DMSO-d₆, 300 MHz): δ 1.03-1.17 (3H, m), 1.24-1.30 (3H, m), 1.32-1.54 (11H, m), 1.73-1.87 (1H, m), 1.93-2.13 (3H, m), 2.17-2.33 (3H, m), 2.55-2.84 (3H, m), 2.91-3.15 (1H, m), 3.22-3.40 (2H, m), 3.70-4.03 (3H, m), 4.10-4.40 (2H, m), 4.65-4.77 (1H, m), 4.91-5.31 (2H, m), 5.79-6.00 (2H, m), 6.72-7.00 (3H, m), 7.00-7.07 (1H, m), 7.10-7.19 (1H, m), 7.19-7.43 (4H, m).

### (iv) Production of (3S,7R,8aR)—N-[(4R)-3,4-dihydro-2H-chromen-4-yl]-7-ethoxy-2-{(2S)-2-[(N-methyl-L-alanyl)amino]-2-phenylacetyl}octahydropyrrolo[1,2-a]pyrazine-3-carboxamide

tert-Butyl[(1S)-2-({(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxo-1-phenylethyl}amino)-1-methyl-2-oxoethyl]methylcarbamate (164 mg) was dissolved in 4M hydrogen chloride-ethyl acetate solution (2 mL), and the solution was stirred at room temperature for 30 min. The reaction mixture was diluted with ethyl acetate (100 mL) and saturated aqueous sodium hydrogen carbonate solution (50 mL), and the aqueous layer was extracted with ethyl acetate (100 mL). The organic layer was dried over anhydrous magnesium sulfate, and the insoluble material was filtered off. The filtrate was concentrated under reduced pressure, and the residue was crystallized from ethyl acetate (3 mL) and hexane (3 mL) to give the title compound (91 mg) as colorless crystals. LC-MS 564.4 (MH⁺)

¹H NMR (DMSO-d₆, 300 MHz): δ 0.98-1.13 (6H, m), 1.21-1.69 (1H, m), 1.82-2.31 (9H, m), 2.68-3.08 (3H, m), 3.25-3.32 (2H, m), 3.41-3.51 (1H, m), 3.85-3.99 (2H, m), 4.14-4.25 (2H, m), 4.40-4.55 (1H, m), 4.93-5.12 (2H, m), 5.64-6.10 (1H, m), 6.69-6.82 (1H, m), 6.83-6.97 (1H, m), 7.08-7.48 (7H, m), 7.99-8.65 (2H, m).

### Example 22

### Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

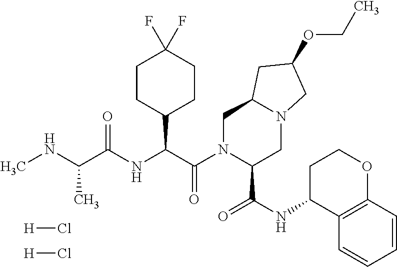

### (i) Production of benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate

tert-Butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate (2.03 g) was dissolved in 4M hydrogen chloride-ethyl acetate solution (30.5 mL), and the solution was stirred at room temperature for 1 hr, and concentrated under reduced pressure. To the residue was added ethyl acetate (10 mL)/diethyl ether (10 mL), and the mixture was stirred at room temperature for 30 min. The precipitate was collected by filtration, washed with ethyl acetate/diethyl ether (=1/1), and dried in vacuum for 1 hr to give (3S,7R,8aR)—N-[(4R)-3,4-dihydro-2H-chromen-4-yl]-7-ethoxyoctahydropyrrolo[1,2-a]pyrazine-3-carboxamide dihydrochloride. (2S)-{[(Benzyloxy)carbonyl]amino}(4,4-difluorocyclohexyl)ethanoic acid (1.64 g) was dissolved in N,N-dimethylformamide (30.5 mL), 1-hydroxybenzotriazole (0.74 g) and N,N-diisopropylethylamine (2.50 mL) were added thereto. 1-Ethyl-3-(3-dimethylaminopropyl)carbodiimide hydrochloride (2.62 g) was added thereto at 0° C., and the mixture was stirred at room temperature for 15 min. (3S,7R,8aR)—N-[(4R)-3,4-Dihydro-2H-chromen-4-yl]-7-ethoxyoctahydropyrrolo[1,2-a]pyrazine-3-carboxamide dihydrochloride was added thereto, and the reaction mixture was stirred at room temperature for 2 days. The mixture was poured into ethyl acetate (300 mL)/water (200 mL), and the organic layer was separated. The organic layer was washed with saturated aqueous sodium hydrogen carbonate (100 mL) and saturated brine (100 mL), dried over anhydrous magnesium sulfate, and filtered. The filtrate was concentrated under reduced pressure, and the residue was purified by silica gel column chromatography (hexane/ethyl acetate=50/50→0/100).

The object fractions were collected, the solvent was evaporated under reduced pressure, and the residue was dried in vacuum for 1 hr to give the title compound (2.43 g) as white crystals.

LC-MS: 655.4 (MH⁺)

¹H NMR (DMSO-d₆, 300 MHz): δ 1.10 (3H, t, J=7.1 Hz), 1.15-1.42 (2H, m), 1.50-2.24 (13H, m), 2.82-3.73 (5H, m), 3.80-4.27 (3H, m), 4.28-5.18 (6H, m), 6.67-6.92 (2H, m), 7.06-7.18 (2H, m), 7.19-7.79 (7H, m), 8.08-8.24 (1H, m).

### (ii) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate

Benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (2.40 g) was dissolved in hydrogen 119 chloride-methanol (48 mL), 10% palladium-carbon (0.72 g, 30 wt %) was added thereto, and the mixture was stirred at room temperature for 3 hr under a hydrogen atmosphere. After nitrogen substitution, the insoluble material was filtered off through celite, and washed with methanol. The filtrate was concentrated under reduced pressure to give a yellow oil. N-(tert-Butoxycarbonyl)-N-methyl-L-alanine (0.82 g) was dissolved in N,N-dimethylformamide (14.4 mL), 1-hydroxybenzotriazole (0.59 g) and N,N-diisopropylethylamine (3.19 mL) were added thereto. 1-Ethyl-3-(3-dimethylaminopropyl)carbodiimide hydrochloride (2.11 g) was added thereto at 0° C., and the mixture was stirred at room temperature for 15 min. A solution of the yellow oil obtained above and N,N-diisopropylethylamine (0.64 mL) in N,N-dimethylformamide (19.2 mL) was added thereto, and the mixture was stirred at room temperature for 16 hr. The reaction mixture was poured into ethyl acetate (200 mL)/water (200 mL), and the organic layer was separated. The organic layer was washed with saturated aqueous sodium hydrogen carbonate (100 mL) and saturated brine (100 mL), dried over anhydrous magnesium sulfate, and filtered. The filtrate was concentrated under reduced pressure, and the residue was purified by silica gel column chromatography (hexane/ethyl acetate=50/50→0/100). The object fractions were collected, the solvent was evaporated under reduced pressure, and the residue was dried in vacuum for 1 hr to give the title compound (1.69 g) as white crystals.

LC-MS: 706.4 (MH⁺)

¹H NMR (DMSO-d₆, 300 MHz): δ 1.10 (3H, t, J=7.1 Hz), 1.10-1.36 (15H, m), 1.55-2.24 (13H, m), 2.57-3.68 (8H, m), 3.86-5.17 (8H, m), 6.70-6.90 (2H, m), 7.07-7.29 (2H, m), 7.65-8.32 (2H, m).

### (iii) Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

To tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (192 mg) was added 4M hydrogen chloride-ethyl acetate solution (5.76 mL), and the mixture was stirred at room temperature for 2 hr, and concentrated under reduced pressure. To the residue was added diethyl ether, and the mixture was stirred at room temperature for 30 min. The precipitate was collected by filtration, washed with heptane/diethyl ether (=1/1), and dried in vacuum to give the title compound (115 mg) as a white powder.

LC-MS: 606.4 (MH⁺)

¹H NMR (DMSO-d₆, 300 MHz): δ 1.12 (3H, t, J=6.9 Hz), 1.20-1.46 (5H, m), 1.50-2.50 (15H, m), 3.19-5.15 (15H, m), 6.74-6.90 (2H, m), 7.08-7.48 (2H, m), 8.62-9.42 (4H, m), 12.20-12.78 (1H, m).

### Example 23

### Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

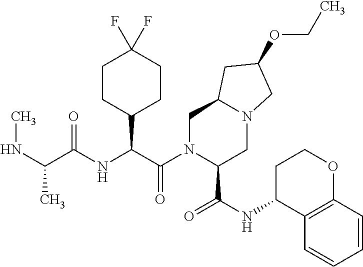

tert-Butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (1.68 g) was dissolved in 4M hydrogen chloride-ethyl acetate solution (33.6 mL), and the solution was stirred at room temperature for 1 hr, and concentrated under reduced pressure. To the residue was added ethyl acetate (80 mL)/tetrahydrofuran (40 mL), and the mixture was washed with saturated aqueous sodium hydrogen carbonate (100 mL) and saturated brine (100 mL), dried over anhydrous magnesium sulfate, and filtered. The filtrate was concentrated under reduced pressure, and the residue was purified by silica gel column chromatography (ethyl acetate/methanol=95/5→70/30). The object fractions were collected, and the solvent was evaporated under reduced pressure. To the residue was added diethyl ether (10 mL), and the mixture was stirred at room temperature for 1 hr. Heptane (10 mL) was added thereto, and the precipitate was collected by filtration, washed with heptane/diethyl ether (=1/1), and dried in vacuum at 50° C. for 3 hr to give the title compound (1.08 g) as white crystals. LC-MS: 606.4 (MH⁺)

¹H NMR (DMSO-d₆, 300 MHz): δ 1.01-1.15 (6H, m), 1.15-1.38 (3H, m), 1.54-2.29 (16H, m), 2.63-3.64 (7H, m), 3.88-4.48 (4H, m), 4.59-5.15 (3H, m), 6.70-6.80 (1H, m), 6.80-6.92 (1H, m), 7.07-7.30 (2H, m), 7.88-8.12 (1H, m), 8.12-8.33 (1H, m).

### Example 24

### Production of (3S,8aR)-2-{(2S)-2-cyclohexyl-2-[(N-methyl-L-alanyl)amino]acetyl}-N-[(4R)-5,7-difluoro-3,4-dihydro-2H-chromen-4-yl]octahydropyrrolo[1,2-a]pyrazine-3-carboxamide dihydrochloride

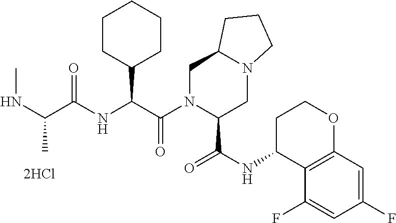

### (i) Production of tert-butyl[(1S)-2-({(1S)-1-cyclohexyl-2-[(3S,8aR)-3-{[(4R)-5,7-difluoro-3,4-dihydro-2H-chromen-4-yl]carbamoyl}hexahydropyrrolo[1,2-a]pyrazin-2(1H)-yl]-2-oxoethyl}amino)-1-methyl-2-oxoethyl]methylcarbamate

To a solution of methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) in THF (6 mL)/water (1.5 mL) was added lithium hydroxide hydrate (26 mg), and the mixture was stirred at 50° C. for 5 hr. The mixture was neutralized with 1M hydrochloric acid (0.62 mL), and concentrated under reduced pressure. To the residue was added toluene, and the mixture was concentrated under reduced pressure, and dried under reduced pressure. The residue was diluted with DMF (8 mL), (4R)-5,7-difluoro-3,4-dihydro-2H-chromen-4-amine hydrochloride (131 mg), 1-hydroxybenzotriazole (53 mg), N-ethyldiisopropylamine (0.137 mL) and N-[3-(dimethylamino)propyl]-N′-ethylcarbodiimide hydrochloride (500 mg) were successively added thereto, and the mixture was stirred at room temperature for 18 hr. The mixture was diluted with ethyl acetate, and washed with water and 5% aqueous sodium bicarbonate. The organic layer was concentrated under reduced pressure, and the residue was subjected to basic silica gel column chromatography (hexane/ethyl acetate=95/5→0/100). The object fractions were concentrated under reduced pressure, and the residue was purified by silica gel column chromatography (ethyl acetate/methanol=100/0→80/20) to give the title compound (256 mg). LC-MS: 662.4 (MH⁺)

### (ii) Production of (3S,8aR)-2-{(2S)-2-cyclohexyl-2-[(N-methyl-L-alanyl)amino]acetyl}-N-[(4R)-5,7-difluoro-3,4-dihydro-2H-chromen-4-yl]octahydropyrrolo[1,2-a]pyrazine-3-carboxamide dihydrochloride

To a solution of tert-butyl[(1S)-2-({(1S)-1-cyclohexyl-2-[(3S,8aR)-3-{[(4R)-5,7-difluoro-3,4-dihydro-2H-chromen-4-yl]carbamoyl}hexahydropyrrolo[1,2-a]pyrazin-2(1H)-yl]-2-oxoethyl}amino)-1-methyl-2-oxoethyl]methylcarbamate (256 mg) in ethyl acetate (5 mL) was added 4M hydrogen chloride-ethyl acetate solution (20 mL), and the mixture was stirred at room temperature for 2 hr. The mixture was concentrated under reduced pressure, ethyl acetate was added to the residue, and the precipitate was collected by filtration, washed with ethyl acetate, and dried under reduced pressure to give the title compound (201 mg). LC-MS: 562.4 (MH⁺)

¹H NMR (DMSO-d₆, 300 MHz): δ 0.90-2.25 (20H, m), 2.40-2.50 (3H, m), 2.90-4.00 (7H, m), 4.00-5.30 (6H, m), 6.55-6.85 (2H, m), 8.40-9.10 (3H, m), 9.20-10.00 (1H, m), 10.30-12.70 (1H, m).

### Example 25

### Production of (3S,8aR)-2-{(2S)-2-cyclohexyl-2-[(N-methyl-L-alanyl)amino]acetyl}-N-[(4R)-5,7-difluoro-3,4-dihydro-2H-chromen-4-yl]octahydropyrrolo[1,2-a]pyrazine-3-carboxamide

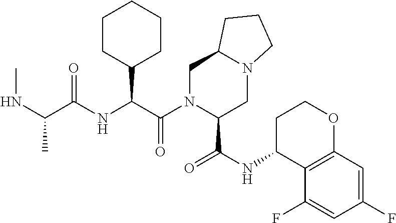

To (3S,8aR)-2-{(2S)-2-cyclohexyl-2-[(N-methyl-L-alanyl)amino]acetyl}-N-[(4R)-5,7-difluoro-3,4-dihydro-2H-chromen-4-yl]octahydropyrrolo[1,2-a]pyrazine-3-carboxamide dihydrochloride (290 mg) were added water (5 mL) and saturated aqueous sodium hydrogen carbonate solution (5 mL), and the mixture was extracted with ethyl acetate (10 mL). The extract was washed with saturated brine, dried over anhydrous magnesium sulfate, and concentrated under reduced pressure to give the title compound (236 mg) as a colorless oil. LC-MS: 562.4 (MH⁺)

¹H NMR (DMSO-d₆, 300 MHz): δ 0.81-1.37 (9H, m), 1.47-2.29 (17H, m), 2.83-3.24 (3H, m), 3.40-3.78 (1H, m), 3.97-4.54 (4H, m), 4.64-4.81 (1H, H), 4.90-5.06 (2H, m), 6.50-6.65 (1H, m), 6.68-6.84 (1H, m), 7.88-8.05 (1H, m), 8.16-8.63 (1H, m).

### Example 26

### Production of (3S,8aR)-2-{(2S)-2-cyclohexyl-2-[(N-methyl-L-alanyl)amino]acetyl}-N-[(1S,2S)-2-hydroxy-2,3-dihydro-1H-inden-1-yl]octahydropyrrolo[1,2-a]pyrazine-3-carboxamide dihydrochloride

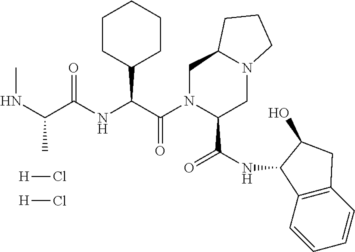

Methyl(3S,8aR)-2-[(2S)-2-{[N-(tert-butoxycarbonyl)-N-methyl-L-alanyl]amino}-2-cyclohexylacetyl]octahydropyrrol[1,2-a]pyrazine-3-carboxylate (300 mg) was dissolved in tetrahydrofuran (8.5 mL), a solution prepared by dissolving lithium hydroxide monohydrate (32 mg) in water (2.1 mL) was added thereto, and the mixture was stirred at 50° C. for 2 hr. Then, lithium hydroxide (7.4 mg) was added thereto, and the mixture was stirred at 50° C. for 3 hr.

The mixture was allowed to cool to room temperature, (1S,2S)-1-amino-2,3-dihydro-1H-inden-2-ol (62 mg) and 4-(4,6-dimethoxy-1,3,5-triazin-2-yl)-4-methylmorpholinium chloride (348 mg) were added to thereto, and the mixture was stirred at room temperature for 16 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a colorless amorphous powder. This amorphous powder was dissolved in ethyl acetate (2 mL), 4M hydrogen chloride-ethyl acetate solution (8 mL) was added thereto, and the mixture was stirred at room temperature for 1 hr. The precipitated solid was collected by filtration, washed with ethyl acetate, and dried under reduced pressure to give the title compound (48 mg) as a colorless amorphous powder. LC-MS: 526.4 (MH⁺)

¹H NMR (DMSO-d₆, 300 MHz): δ 1.02-2.15 (20H, m), 2.46 (3H, t, J=5.1 Hz), 2.64-2.80 (1H, m), 3.04-3.13 (1H, m), 3.55-4.99 (11H, m), 6.36-8.05 (4H, m), 8.15-10.50 (4H, m), 11.94-12.70 (1H, m).

### Example 27

### Production of (3S,8aR)-2-{(2S)-2-cyclohexyl-2-[(N-methyl-L-alanyl)amino]acetyl}-N-[(1S,2R)-2-hydroxy-2,3-dihydro-1H-inden-1-yl]octahydropyrrolo[1,2-a]pyrazine-3-carboxamide dihydrochloride

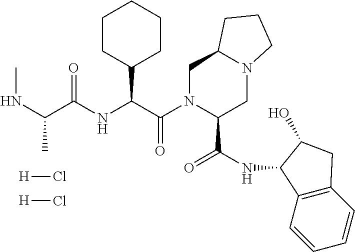

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (8.5 mL), a solution prepared by dissolving lithium hydroxide monohydrate (39 mg) in water (2.1 mL) was added thereto, and the mixture was stirred at 50° C. for 4 hr. The mixture was allowed to cool to room temperature, (1S,2R)-1-amino-2,3-dihydro-1H-inden-2-ol (89 mg) and 4-(4,6-dimethoxy-1,3,5-triazin-2-yl)-4-methylmorpholinium chloride (348 mg) were added thereto, and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a colorless amorphous powder. This amorphous powder was dissolved in ethyl acetate (4 mL), 4M hydrogen chloride-ethyl acetate solution (4 mL) was added thereto, and the mixture was stirred at room temperature for 2 hr. The precipitated solid was collected by filtration, washed with ethyl acetate, and dried under reduced pressure to give the title compound (18 mg) as a colorless amorphous powder. LC-MS: 526.4 (MH⁺)

¹H NMR (DMSO-d₆, 300 MHz): δ 0.94-2.21 (19H, m), 2.40-2.48 (3H, m), 3.05 (2H, dd, J=16.1, 4.9 Hz), 3.48-3.97 (6H, m), 4.29-4.92 (4H, m), 4.96-5.56 (2H, m), 7.07-7.32 (4H, m), 8.26-8.56 (1H, m), 8.67-9.01 (2H, m), 9.21-9.65 (1H, m), 11.86-12.43 (1H, m).

### Example 28

Production of (3S,8aR)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide

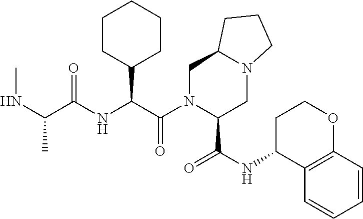

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (1.80 g) was dissolved in tetrahydrofuran (40 mL), a solution prepared by dissolving lithium hydroxide monohydrate (193 mg) in water (10 mL) was added thereto, and the mixture was stirred at 50° C. for 3 hr. The mixture was allowed to cool to room temperature, 1M hydrochloric acid (4.6 mL) was added thereto, and the mixture was concentrated under reduced pressure to give a colorless amorphous powder. This amorphous powder, (4R)-3,4-dihydro-2H-chromen-4-amine hydrochloride (984 mg), 1-ethyl-3-(3-dimethylaminopropyl)carbodiimide hydrochloride (4.48 g), 1-hydroxybenzotriazole (478 mg) and N,N-diisopropylethylamine (1.23 mL) were mixed in N,N-dimethylformamide (20 mL), and the mixture was stirred at room temperature for 48 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=10/90→100/0) and then basic silica gel column chromatography (ethyl acetate/hexane=30/70→100/0) to give tert-butyl[(1S)-2-({(1S)-1-cyclohexyl-2-[(3S,8aR)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]methylcarbamate as a colorless oil. This colorless oil was dissolved in ethyl acetate (16 mL), 4M hydrogen chloride-ethyl acetate solution (8 mL) was added thereto, and the mixture was stirred at room temperature for 4 hr. To the mixture was added water (30 mL), and the aqueous layer was separated. The aqueous layer was neutralized with 1M aqueous sodium hydroxide solution, and extracted with ethyl acetate (30 mL). The organic layer was dried over anhydrous magnesium sulfate, and concentrated under reduced pressure to give the title compound (912 mg) as a colorless oil. LC-MS: 526.4 (MH⁺)

¹H NMR (DMSO-d₆, 300 MHz): δ 0.84-1.36 (9H, m), 1.49-2.05 (12H, m), 2.18 (3H, s), 2.84-3.04 (2H, m), 3.16-3.69 (5H, m), 4.07-4.60 (3H, m), 4.66-4.80 (1H, m), 4.91-5.12 (2H, m), 6.71-6.93 (2H, m), 7.08-7.27 (2H, m), 7.85-8.05 (1H, m), 8.12-8.35 (1H, m).

### Example 29

### Production of (3S,8aR)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

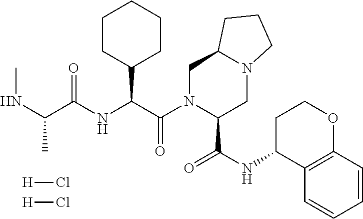

(3S,8aR)-2-{(2S)-2-Cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide (902 mg) was dissolved in ethyl acetate (10 mL), 4M hydrogen chloride-ethyl acetate solution (1 mL) was added thereto, and the mixture was stirred at room temperature for 3 hr. The precipitated solid was collected by filtration, washed with ethyl acetate, and dried under reduced pressure to give the title compound (1.01 g) as a colorless amorphous powder. LC-MS: 526.4 (MH⁺)

¹H NMR (DMSO-d₆, 300 MHz): δ 0.95-1.27 (5H, m), 1.37 (3H, d, J=6.8 Hz), 1.50-2.18 (12H, m), 2.41-2.48 (3H, m), 2.95-3.78 (6H, m), 3.81-3.94 (1H, m), 4.20-4.53 (3H, m), 4.58-4.68 (1H, m), 4.71-4.81 (1H, m), 4.92-5.04 (1H, m), 6.73-6.95 (2H, m). 7.10-7.62 (2H, m), 8.48-9.20 (3H, m), 9.24-10.44 (1H, m). 11.95-12.60 (1H, m).

### Example 30

### Production of (3S,8aR)—N-[(4R)-3,4-dihydro-2H-chromen-4-yl]-2-[(2S)-2-[(N-methyl-L-alanyl)amino]-2-(tetrahydro-2H-pyran-4-yl)acetyl]octahydropyrrolo[1,2-a]pyrazine-3-carboxamide dihydrochloride

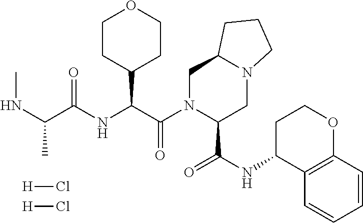

### (i) Production of benzyl[(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-(tetrahydro-2H-pyran-4-yl)ethyl]carbamate

tert-Butyl(3S,8aR)-3-[(4R)-3,4-dihydro-2H-chromen-4-ylcarbamoyl]hexahydropyrrolo[1,2-a]pyrazine-2(1H)-carboxylate (450 mg) was dissolved in ethyl acetate (3 mL), 4M hydrogen chloride-ethyl acetate solution (3 mL) was added thereto, and the mixture was stirred at room temperature for 1 hr. The mixture was concentrated under reduced pressure, and the residue was collected by filtration, washed with ether, and dried under reduced pressure to give a colorless amorphous powder (410 mg). The obtained colorless amorphous powder (200 mg), (2S)-{[(benzyloxy)carbonyl]amino}(tetrahydro-2H-pyran-4-yl)ethanoic acid (235 mg), O-(7-azabenzotriazol-1-yl)-N,N,N′,N′-tetramethyluronium hexafluorophosphate (406 mg) and N,N-diisopropylethylamine (0.558 mL) were mixed in N,N-dimethylformamide (5 mL), and the mixture was stirred at room temperature for 18 hr. The mixture was diluted with ethyl acetate (30 mL), and washed with water (30 mL), saturated aqueous sodium hydrogen carbonate solution (50 mL) and saturated brine (50 mL). The organic layer was dried over anhydrous magnesium sulfate, and concentrated under reduced pressure. The residue was purified by silica gel column chromatography (ethyl acetate/hexane=15/85→100/0) to give the title compound (200 mg) as a colorless amorphous powder. LC-MS: 577 (MH⁺)

### (ii) Production of tert-butyl[(1S)-2-{[(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-(tetrahydro-2H-pyran-4-yl)ethyl]amino}-1-methyl-2-oxoethyl]methylcarbamate

To a solution of benzyl[(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-(tetrahydro-2H-pyran-4-yl)ethyl]carbamate (200 mg) in 5-10% hydrogen chloride-methanol (10 mL) was added 10% palladium-carbon (40 mg, 50% water-containing product), and the mixture was stirred at room temperature for 1 hr under a hydrogen atmosphere (2 atm). The insoluble material was filtered off through a celite pad, and the filtrate was concentrated to give (3S,8aR)-2-[(2S)-2-amino-2-(tetrahydro-2H-pyran-4-yl)acetyl]-N-[(4R)-3,4-dihydro-2H-chromen-4-yl]octahydropyrrolo[1,2-a]pyrazine-3-carboxamide dihydrochloride as a colorless oil. The obtained (3S,8aR)-2-[(2S)-2-amino-2-(tetrahydro-2H-pyran-4-yl)acetyl]-N-[(4R)-3,4-dihydro-2H-chromen-4-yl]octahydropyrrolo[1,2-a]pyrazine-3-carboxamide dihydrochloride, N-(tert-butoxycarbonyl)-N-methyl-L-alanine (106 mg), O-(7-azabenzotriazol-1-yl)-N,N,N′,N′-tetramethyluronium hexafluorophosphate (264 mg) and N,N-diisopropylethylamine (0.363 mL) were mixed in N,N-dimethylformamide (5 mL), and the mixture was stirred at room temperature for 18 hr. The mixture was diluted with ethyl acetate (30 mL), and washed with water (30 mL), saturated aqueous sodium hydrogen carbonate solution (50 mL) and saturated brine (50 mL). The organic layer was dried over anhydrous magnesium sulfate, and concentrated under reduced pressure. The residue was purified by silica gel column chromatography (ethyl acetate/hexane=10/90→100/0) to give the title compound (180 mg) as a colorless amorphous powder. LC-MS: 628.4 (MH⁺)

### (iii) Production of (3S,8aR)—N-[(4R)-3,4-dihydro-2H-chromen-4-yl]-2-[(2S)-2-[(N-methyl-L-alanyl)amino]-2-(tetrahydro-2H-pyran-4-yl)acetyl]octahydropyrrolo[1,2-a]pyrazine-3-carboxamide dihydrochloride

tert-Butyl[(1S)-2-{[(1S)-2-[(3S,8aR)-3-[(4R)-3,4-dihydro-2H-chromen-4-ylcarbamoyl]hexahydropyrrolo[1,2-a]pyrazin-2(1H)-yl]-2-oxo-1-(tetrahydro-2H-pyran-4-yl)ethyl]amino}-1-methyl-2-oxoethyl]methylcarbamate (180 mg) was dissolved in ethyl acetate (3 mL), 4M hydrogen chloride-ethyl acetate solution (3 mL) was added thereto, and the mixture was stirred at room temperature for 2 hr. The mixture was concentrated under reduced pressure, and the residue was collected by filtration, washed with ether, and dried under reduced pressure to give the title compound (150 mg) as a colorless amorphous powder. LC-MS: 528.4 (MH⁺)

¹H NMR (DMSO-d₆, 300 MHz): δ 1.38 (6H, d, J=6.8 Hz), 1.50-2.22 (11H, m), 3.05 (1H, brs), 3.37 (3H, s), 3.41-3.72 (6H, m), 4.19 (3H, t, J=5.0 Hz), 4.40-4.73 (2H, m), 4.77-5.03 (2H, m), 6.63-6.96 (2H, m), 7.02-7.26 (2H, m), 8.51-9.07 (3H, m), 9.14-9.54 (1H, m), 12.08 (1H, brs).

### Example 31

### Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide

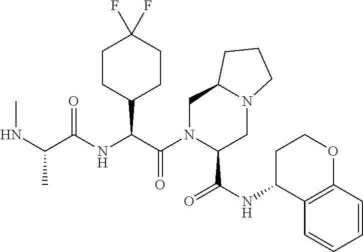

### (i) Production of tert-butyl(3S,8aR)-3-[(4R)-3,4-dihydro-2H-chromen-4-ylcarbamoyl]hexahydropyrrolo[1,2-a]pyrazine-2(1H)-carboxylate

2-tert-Butyl 3-methyl(3S,8aR)-hexahydropyrrolo[1,2-a]pyrazine-2,3(1H)-dicarboxylate (852 mg) was dissolved in tetrahydrofuran (6 mL), a solution prepared by dissolving lithium hydroxide monohydrate (164 mg) in water (2 mL) was added thereto, and the mixture was stirred at 50° C. for 3 hr. To the mixture was added 1M hydrochloric acid (3.9 mL), and the mixture was concentrated under reduced pressure to give a colorless amorphous powder. This amorphous powder, (4R)-3,4-dihydro-2H-chromen-4-amine hydrochloride (863 mg), 1-ethyl-3-(3-dimethylaminopropyl)carbodiimide hydrochloride (1.73 g), 1-hydroxybenzotriazole (486 mg) and N,N-diisopropylethylamine (1.57 mL) were mixed in N,N-dimethylformamide (10 mL), and the mixture was stirred at room temperature for 15 hr. The mixture was diluted with ethyl acetate (50 mL), and washed with water (100 mL) and saturated brine (100 mL). The organic layer was dried over anhydrous magnesium sulfate, and concentrated under reduced pressure. The residue was purified by silica gel column chromatography (ethyl acetate/hexane=50/50→100/0) to give the title compound (1.12 g) as a white solid. LC-MS: 402.2 (MH⁺)

### (ii) Production of (3S,8aR)—N-[(4R)-3,4-dihydro-2H-chromen-4-yl]octahydropyrrolo[1,2-a]pyrazine-3-carboxamide

tert-Butyl(3S,8aR)-3-[(4R)-3,4-dihydro-2H-chromen-4-ylcarbamoyl]hexahydropyrrolo[1,2-a]pyrazine-2(1H)-carboxylate (1.12 g) was dissolved in ethyl acetate (14 mL), 4M hydrogen chloride-ethyl acetate solution (7 mL) was added thereto, and the mixture was stirred at room temperature for 1 hr. Then, methanol (4 mL) was added thereto, and the mixture was stirred at 40° C. for 4 hr. To the mixture was added water (10 mL), and the aqueous layer was separated. The aqueous layer was neutralized with saturated aqueous sodium hydrogen carbonate solution, and the mixture was extracted with ethyl acetate (100 mL). The organic layer was washed with saturated brine (100 mL), dried over anhydrous magnesium sulfate, and concentrated under reduced pressure. The residue was purified by silica gel column chromatography (ethyl acetate/methanol=100/0→90/10) to give the title compound (768 mg) as a white solid. LC-MS: 302.2 (MH⁺)

¹H NMR (DMSO-d₆, 300 MHz): δ 1.06-1.23 (1H, m), 1.49-1.78 (4H, m), 1.89-2.08 (4H, m), 2.26-2.40 (1H, m), 2.69-2.84 (2H, m), 2.93 (1H, td, J=8.2, 2.4 Hz), 3.59 (1H, dd, J=10.4, 1.1 Hz), 4.14-4.24 (2H, m), 4.97-5.09 (1H, m), 6.76 (1H, d, J=8.1 Hz), 6.85 (1H, td, J=7.5, 0.9 Hz), 7.08-7.19 (2H, m), 8.07 (1H, d, J=8.7 Hz)

### (iii) Production of benzyl{(1S)-1-(4,4-difluorocyclohexyl)-2-[(3S,8aR)-3-[(4R)-3,4-dihydro-2H-chromen-4-ylcarbamoyl]hexahydropyrrolo[1,2-a]pyrazin-2(1H)-yl]-2-oxoethyl}carbamate

(3S,8aR)—N-[(4R)-3,4-Dihydro-2H-chromen-4-yl]octahydropyrrolo[1,2-a]pyrazine-3-carboxamide (755 mg), (2S)-(4,4-difluorocyclohexyl)[(benzyloxycarbonyl)amino]ethanoic acid (985 mg), O-(benzotriazol-1-yl)-N,N,N′,N′-tetramethyluronium hexafluorophosphate (1.71 g) and N,N-diisopropylethylamine (1.57 mL) were mixed in N,N-dimethylformamide (10 mL), and the mixture was stirred at room temperature for 2 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=30/70→100/0) and then basic silica gel column chromatography (ethyl acetate/hexane=5/95→50/50) to give the title compound (1.51 g) as a colorless oil. LC-MS: 611.3 (MH⁺)

¹H NMR (DMSO-d₆, 300 MHz): δ 1.12-1.41 (3H, m), 1.51-2.07 (14H, m), 2.09-2.20 (1H, m), 2.87-3.21 (2H, m), 3.46-3.73 (1H, m), 3.94-4.26 (3H, m), 4.32-4.56 (1H, m), 4.86-5.12 (4H, m), 6.71-6.79 (1H, m), 6.80-6.91 (1H, m), 7.07-7.45 (7H, m), 7.63 (1H, d, J=8.9 Hz), 8.06-8.22 (1H, m).

### (iv) Production of (3S,8aR)-2-[(2S)-2-amino-2-(4,4-difluorocyclohexyl)acetyl]-N-[(4R)-3,4-dihydro-2H-chromen-4-yl]octahydropyrrolo[1,2-a]pyrazine-3-carboxamide

Benzyl{(1S)-1-(4,4-difluorocyclohexyl)-2-[(3S,8aR)-3-[(4R)-3,4-dihydro-2H-chromen-4-ylcarbamoyl]hexahydropyrrolo[1,2-a]pyrazin-2(1H)-yl]-2-oxoethyl}carbamate (1.50 g) was dissolved in methanol (15 mL) and tetrahydrofuran (15 mL), 10% palladium-carbon (300 mg, 50% water-containing product) was added thereto, and the mixture was stirred at room temperature for 4 hr under a hydrogen atmosphere (3 atm). The insoluble material was filtered off through a celite pad, and the filtrate was concentrated to give the title compound (1.13 g) as a colorless oil. LC-MS: 477.3 (MH⁺)

¹H NMR (DMSO-d₆, 300 MHz): δ 1.14-2.24 (20H, m), 2.87-3.05 (1H, m), 3.14-3.27 (1H, m), 3.37-3.51 (1H, m), 3.64 (1H, d, J=5.9 Hz), 3.98-4.27 (3H, m), 4.45-5.13 (2H, m), 6.71-6.93 (2H, m), 7.07-7.21 (2H, m), 7.91-8.42 (1H, m).

### (v) Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide

(3S,8aR)-2-[(2S)-2-Amino-2-(4,4-difluorocyclohexyl)acetyl]-N-[(4R)-3,4-dihydro-2H-chromen-4-yl]octahydropyrrolo[1,2-a]pyrazine-3-carboxamide (1.12 g), N-(tert-butoxycarbonyl)-N-methyl-L-alanine (716 mg), O-(benzotriazol-1-yl)-N,N,N′,N′-tetramethyluronium hexafluorophosphate (1.79 g) and N,N-diisopropylethylamine (1.64 mL) were mixed in N,N-dimethylformamide (20 mL), and the mixture was stirred at room temperature for 15 hr. The mixture was diluted with ethyl acetate (50 mL), and washed with water (50 mL) and saturated brine (50 mL). The organic layer was dried over anhydrous magnesium sulfate, and concentrated under reduced pressure. The residue was purified by basic silica gel column chromatography (ethyl acetate/hexane=20/80→100/0) and then silica gel column chromatography (ethyl acetate/hexane=50/50→100/0) to give a colorless oil. This colorless oil was dissolved in ethyl acetate (8 mL), 4M hydrogen chloride-ethyl acetate solution (8 mL) was added thereto, and the mixture was stirred at room temperature for min. Then, methanol (2 mL) was added thereto, and the mixture was stirred at room temperature for 2 hr. To the mixture was added water (20 mL), and the aqueous layer was separated. The aqueous layer was neutralized with 8M aqueous sodium hydroxide solution, and the mixture was extracted with ethyl acetate (30 mL). The organic layer was washed with saturated brine (100 mL), dried over anhydrous magnesium sulfate, and concentrated under reduced pressure. The residue was purified by basic silica gel column chromatography (ethyl acetate/methanol=100/0→90/10) to give the title compound (1.08 g) as a colorless oil. LC-MS: 562.3 (MH⁺)

¹H NMR (DMSO-d₆, 300 MHz): δ 1.01-1.38 (6H, m), 1.44-2.26 (19H, m), 2.87-3.03 (2H, m), 3.13-3.26 (1H, m), 3.44-3.70 (1H, m), 3.97-4.09 (1H, m), 4.12-4.26 (2H, m), 4.37-5.14 (3H, m), 6.71-6.93 (2H, m), 7.06-7.27 (2H, m), 7.90-8.35 (2H, m).

### Example 32

### Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

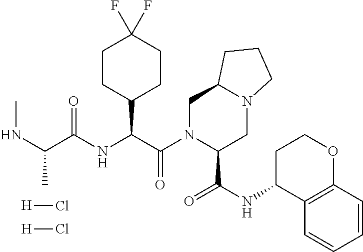

(3S,8aR)-2-{(2S)-2-(4,4-Difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide (900 mg) was dissolved in ethyl acetate (10 mL), 4M hydrogen chloride-ethyl acetate solution (1.2 mL) was added thereto, and the mixture was stirred at room temperature for 4 hr. The precipitated solid was collected by filtration, washed with ethyl acetate, and dried under reduced pressure to give the title compound (922 mg) as a colorless amorphous powder. LC-MS: 562.3 (MH⁺)

¹H NMR (DMSO-d₆, 300 MHz): δ 1.23-1.48 (5H, m), 1.58-2.22 (14H, m), 2.41-2.48 (3H, m), 2.92-3.18 (1H, m), 3.47-4.29 (7H, m), 4.44-4.75 (2H, m), 4.80-5.06 (2H, m), 6.58-7.54 (4H, m), 8.50-9.28 (3H, m), 9.33-9.73 (1H, m), 11.09-12.69 (1H, m).

### Example 33

### Production of (3S,7R,8aR)-2-{(2S)-2-cyclohexyl-2-[(N-methyl-L-alanyl)amino]acetyl}-N-[(4R)-7-fluoro-3,4-dihydro-2H-chromen-4-yl]-7-(2,2,2-trifluoroethoxy)octahydropyrrolo[1,2-a]pyrazine-3-carboxamide

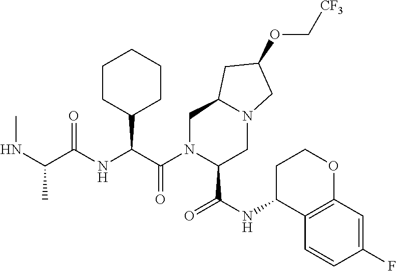

### (i) Production of tert-butyl(2R,4R)-2-(benzylcarbamoyl)-4-hydroxypyrrolidine-1-carboxylate

To a solution of (4R)-1-(tert-butoxycarbonyl)-4-hydroxy-D-proline (50.0 g) in acetonitrile (750 mL) were added benzylamine (28.3 mL), N,N-dimethyl-4-aminopyridine (2.64 g), 1-hydroxybenzotriazole (38.0 g) and N-[3-(dimethylamino)propyl]-N′-ethylcarbodiimide hydrochloride (54.0 mg), and the mixture was stirred at room temperature for 14 hr. To the reaction mixture was added 5% aqueous citric acid solution (750 mL), and the mixture was extracted with ethyl acetate (1.5 L). The organic layer was washed with saturated aqueous sodium hydrogen carbonate solution (750 mL) and saturated brine (500 mL), and dried over anhydrous magnesium sulfate, and the insoluble material was filtered off. The filtrate was concentrated under reduced pressure, and the residue was charged on a pad filled with basic silica gel, and eluted with ethyl acetate (4.0 L). The fraction containing the title compound was concentrated under reduced pressure to give the title compound (62.3 g) as a colorless solid. LC-MS: 321.2 (MH⁺)

¹H NMR (DMSO-d₆, 300 MHz): δ 1.22-1.47 (9H, m), 1.69-1.82 (1H, m), 2.24-2.41 (1H, m), 3.15-3.26 (1H, m), 3.49 (1H, dd, J=10.7, 5.4 Hz), 4.07-4.42 (4H, 4, 5.18-5.34 (1H, m), 7.18-7.37 (5H, m), 8.48 (1H, t, J=6.0 Hz).

### (ii) Production of (4R)—N-benzyl-4-hydroxy-D-prolinamide

tert-Butyl(2R,4R)-2-(benzylcarbamoyl)-4-hydroxypyrrolidine-1-carboxylate (61.0 g) was dissolved in 4M hydrogen chloride-ethyl acetate solution (500 mL), and the mixture was stirred at room temperature for 2 hr. The solvent was evaporated under reduced pressure, and the residue was charged on a pad filled with Amberlyst A-21, and eluted with methanol (1.0 L). The fraction containing the title compound was concentrated under reduced pressure to give the title compound (42.8 g) as a colorless solid. LC-MS: 221.1 (MH⁺)

¹H NMR (DMSO-d₆, 300 MHz): δ 1.78 (1H, ddd, J=13.2, 5.9, 4.8 Hz), 2.30 (1H, ddd, J=13.2, 9.4, 5.8 Hz), 2.87 (1H, dd, J=11.2, 3.1 Hz), 3.04 (1H, dd, J=11.2, 5.1 Hz), 3.17 (1H, s), 3.86 (1H, dd, J=9.4, 5.9 Hz), 4.17-4.28 (1H, m), 4.32 (2H, d, J=5.9 Hz), 5.00 (1H, br s), 7.16-7.40 (5H, m), 8.69 (1H, t, J=5.9 Hz).

### (iii) Production of (3R,5R)-5-[(benzylamino)methyl]pyrrolidin-3-ol

To a suspension of lithium aluminum hydride (12.9 g) in tetrahydrofuran (300 mL) was added dropwise a suspension of (4R)—N-benzyl-4-hydroxy-D-prolinamide (37.4 g) in tetrahydrofuran under ice-cooling, and the mixture was heated to 80° C. and stirred with heating for 1.4 hr. The reaction mixture was cooled to 0° C., water (13 mL), 1M aqueous sodium hydroxide solution (13 mL) and water (26 mL) were added thereto, and the insoluble material was filtered off. The filtrate was concentrated under reduced pressure to give the title compound (30.0 g) as a yellow oil. Without further purification, the compound was used for the next reaction. LC-MS: 207.1 (MH⁺)

### (iv) Production of methyl(3S,7R,8aR)-2-benzyl-7-hydroxyoctahydropyrrolo[1,2-a]pyrazine-3-carboxylate

To a solution of (3R,5R)-5-[(benzylamino)methyl]pyrrolidin-3-ol (33.0 g) in toluene (330 mL) were added triethylamine (60 mL) and methyl 2,3-dibromopropanoate (18.2 mL), and the mixture was stirred with heating at 80° C. for 20 hr. The reaction mixture was allowed to cool to room temperature, and diluted with ethyl acetate (500 mL) and water (300 mL), and the aqueous layer was extracted with ethyl acetate (500 mL). The organic layer was dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (12.6 g) as an orange oil. LC-MS: 291.2 (MH⁺)

¹H NMR (DMSO-d₆, 300 MHz): δ 1.06-1.21 (1H, m), 1.79-1.94 (1H, m), 1.99-2.14 (2H, m), 2.24 (1H, dd, J=10.6, 4.0 Hz), 2.59 (1H, dd, J=10.7, 3.0 Hz), 2.74 (1H, d, J=9.8 Hz), 2.79-2.89 (1H, m), 3.24 (1H, dd, J=10.7, 1.7 Hz), 3.49-3.56 (1H, m), 3.64 (3H, s), 3.89 (2H, s), 4.14 (1H, dt J=6.75, 3.14 Hz), 4.70 (1H, d, J=4.5 Hz), 7.18-7.38 (5H, m).

### (v) Production of 2-tert-butyl 3-methyl(3S,7R,8aR)-7-hydroxyhexahydropyrrolo[1,2-a]pyrazine-2,3(1H)-dicarboxylate

Methyl(3S,7R,8aR)-2-benzyl-7-hydroxyoctahydropyrrolo[1,2-a]pyrazine-3-carboxylate (12.0 g) was dissolved in 5-10% hydrogen chloride-methanol solution (120 mL), 10% palladium-carbon (1.2 g, 50% water-containing product) was added thereto, and the mixture was stirred at room temperature for 3 hr under a hydrogen atmosphere. The insoluble material was filtered off through a pad filled with celite, and the pad was washed with methanol (300 mL). The filtrate and the washing solution were concentrated under reduced pressure, and the residue was suspended in tetrahydrofuran (180 mL). Saturated aqueous sodium hydrogen carbonate solution (180 mL) and di-tert-butyl dicarbonate (9.93 g) were added thereto, and the mixture was stirred at room temperature for 18 hr. The reaction mixture was diluted with ethyl acetate (300 mL), and the aqueous layer was extracted with a mixed solvent (300 mL) of ethyl acetate/tetrahydrofuran (3/1). The organic layer was dried over anhydrous magnesium sulfate, and the insoluble material was filtered off. The filtrate was concentrated under reduced pressure, and the residue was purified by silica gel column chromatography (ethyl acetate/methanol=100/0→80/20) to give the title compound (10.7 g) as a colorless solid. LC-MS: 301.2 (MH⁺)

¹H NMR (DMSO-d₆, 300 MHz): δ 1.06-1.22 (1H, m), 1.35-1.41 (9H, m), 1.70-1.85 (1H, m), 2.07-2.26 (3H, m), 2.58-2.88 (2H, m), 3.28-3.41 (1H, m), 3.67-3.69 (3H, m), 3.79-3.92 (1H, m), 4.11-4.24 (1H, m), 4.53-4.66 (1H, m), 4.77 (1H, d J=4.2 Hz).

### (vi) Production of tert-butyl(3S,7R,8aR)-3-{[(4R)-7-fluoro-3,4-dihydro-2H-chromen-4-yl]carbamoyl}-7-hydroxyhexahydropyrrolo[1,2-a]pyrazine-2(1H)-carboxylate

2-tert-Butyl 3-methyl(3S,7R,8aR)-7-hydroxyhexahydropyrrolo[1,2-a]pyrazine-2,3(1H)-dicarboxylate (600 mg) was dissolved in tetrahydrofuran (6 mL), a solution prepared by dissolving lithium hydroxide monohydrate (109 mg) in water (2 mL) was added thereto, and the mixture was stirred at 50° C. for 3 hr. To the mixture was added 1M hydrochloric acid (2.6 mL), and the mixture was concentrated under reduced pressure to give a colorless amorphous powder. This amorphous powder, (4R)-7-fluoro-3,4-dihydro-2H-chromen-4-amine hydrochloride (369 mg), 1-ethyl-3-(3-dimethylaminopropyl)carbodiimide hydrochloride (1.15 g), 1-hydroxybenzotriazole (324 mg) and N,N-diisopropylethylamine (1.05 mL) were mixed in N,N-dimethylformamide (5 mL), and the mixture was stirred at room temperature for 15 hr. The mixture was diluted with ethyl acetate (50 mL), and washed with saturated aqueous sodium hydrogen carbonate solution (100 mL) and saturated brine (100 mL). The organic layer was dried over anhydrous magnesium sulfate, and concentrated under reduced pressure. The residue was collected by filtration, and washed with ethyl acetate-hexane to give the title compound (760 mg) as a white solid. LC-MS: 436.2 (MH⁺)

¹H NMR (DMSO-d₆, 300 MHz): δ 1.09-1.25 (1H, m), 1.30-1.48 (9H, m), 1.66-1.81 (1H, m), 1.84-2.24 (5H, m), 2.78 (1H, d, J=9.8 Hz), 2.86-3.10 (1H, m), 3.27-3.47 (1H, m), 3.77-3.92 (1H, m), 4.11-4.31 (3H, m), 4.34-4.53 (1H, m), 4.73-4.81 (1H, m), 4.98-5.11 (1H, m), 6.58-6.77 (2H, m), 7.13-7.27 (1H, m), 8.16-8.39 (1H, m).

### (vii) Production of tert-butyl(3S,7R,8aR)-3-{[(4R)-7-fluoro-3,4-dihydro-2H-chromen-4-yl]carbamoyl}-7-(2,2,2-trifluoroethoxy)hexahydropyrrolo[1,2-a]pyrazine-2(1H)-carboxylate

To a suspension of sodium hydride (60%, 36.4 mg) in tetrahydrofuran (5 mL) was added tert-butyl(3S,7R,8aR)-3-{[(4R)-7-fluoro-3,4-dihydro-2H-chromen-4-yl]carbamoyl}-7-hydroxyhexahydropyrrolo[1,2-a]pyrazine-2(1H)-carboxylate (360 mg) under ice-cooling. The mixture was stirred at room temperature for 30 min, 2,2,2-trifluoroethyl trifluoromethanesulfonate (358 μL) was added thereto, and the mixture was stirred at 60° C. for 3 hr. Then, 2,2,2-trifluoroethyl trifluoromethanesulfonate (358 μL) was added thereto, and the mixture was stirred at 60° C. for 6 hr. The reaction mixture was allowed to cool to room temperature, diluted with ethyl acetate (30 mL), and washed with 0.01M hydrochloric acid (30 mL) and saturated brine (30 mL). The organic layer was dried over anhydrous magnesium sulfate, and concentrated under reduced pressure. The residue was purified by silica gel column chromatography (ethyl acetate/hexane=30/70→100/0) to give the title compound (176 mg) as a colorless oil. LC-MS: 518.3 (MH⁺)

¹H NMR (DMSO-d₆, 300 MHz): δ 1.13-1.26 (1H, m), 1.32-1.44 (9H, m), 1.47-1.64 (1H, m), 1.74-2.15 (5H, m), 2.23-2.37 (1H, m), 2.71-3.06 (1H, m), 3.26-3.45 (1H, m), 3.81-4.09 (3H, m), 4.14-4.29 (3H, m), 4.33-4.54 (1H, m), 4.93-5.10 (1H, m), 6.58-6.76 (2H, m), 7.07-7.20 (1H, m), 8.17-8.40 (1H, m).

### (viii) Production of (3S,7R,8aR)—N-[(4R)-7-fluoro-3,4-dihydro-2H-chromen-4-yl]-7-(2,2,2-trifluoroethoxy)octahydropyrrolo[1,2-a]pyrazine-3-carboxamide

tert-Butyl(3S,7R,8aR)-3-{[(4R)-7-fluoro-3,4-dihydro-2H-chromen-4-yl]carbamoyl}-7-(2,2,2-trifluoroethoxy)hexahydropyrrolo[1,2-a]pyrazine-2(1H)-carboxylate (165 mg) was dissolved in ethyl acetate (4 mL), 4M hydrogen chloride-ethyl acetate solution (4 mL) was added thereto, and the mixture was stirred at room temperature for 3 hr. To the mixture was added water (10 mL) and saturated aqueous sodium hydrogen carbonate solution (20 mL), and the mixture was extracted with ethyl acetate (20 mL), and washed with saturated brine (20 mL). The organic layer was dried over anhydrous magnesium sulfate, and concentrated under reduced pressure. The residue was collected by filtration, and washed with ethyl acetate-hexane to give the title compound (92 mg) as a white solid. LC-MS: 418.2 (MH⁺)

¹H NMR (DMSO-d₆, 300 MHz): δ 1.34-1.49 (1H, m), 1.65-1.79 (1H, m), 1.93-2.05 (4H, m), 2.11 (1H, dd, J=10.4, 4.2 Hz), 2.21-2.33 (1H, m), 2.71-3.07 (2H, m), 3.27-3.42 (2H, m), 3.52 (1H, d, J=10.0 Hz), 3.90-4.09 (2H, m), 4.12-4.27 (3H, m), 4.93-5.07 (1H, m), 6.62 (1H, dd, J=10.6, 2.6 Hz), 6.72 (1H, td, J=8.6, 2.6 Hz), 7.08-7.18 (1H, m), 8.08 (1H, d, J=8.5 Hz).

### (ix) Production of (3S,7R,8aR)-2-[(2S)-2-amino-2-cyclohexylacetyl]-N-[(4R)-7-fluoro-3,4-dihydro-2H-chromen-4-yl]-7-(2,2,2-trifluoroethoxy)octahydropyrrolo[1,2-a]pyrazine-3-carboxamide

(3S,7R,8aR)—N-[(4R)-7-Fluoro-3,4-dihydro-2H-chromen-4-yl]-7-(2,2,2-trifluoroethoxy)octahydropyrrolo[1,2-a]pyrazine-3-carboxamide (85.0 mg), (2S)-[(tert-butoxycarbonyl)amino] (cyclohexyl)ethanoic acid (62.9 mg), 0-(benzotriazol-1-yl)-N,N,N′,N′-tetramethyluronium hexafluorophosphate (139 mg) and N,N-diisopropylethylamine (128 μl) were mixed in N,N-dimethylformamide (3 mL), and the mixture was stirred at room temperature for 2 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=30/70→100/0) to give a colorless oil. This colorless oil was dissolved in ethyl acetate (2 mL), 4M hydrogen chloride-ethyl acetate solution (2 mL) was added thereto, and the mixture was stirred at room temperature for 4 hr. To the mixture was added saturated aqueous sodium hydrogen carbonate solution (20 mL), and the mixture was extracted with ethyl acetate (20 mL), and washed with saturated brine (20 mL). The organic layer was dried over anhydrous magnesium sulfate, and concentrated under reduced pressure. The residue was purified by silica gel column chromatography (ethyl acetate/methanol=100/0→80/20) to give the title compound (99.8 mg) as a colorless oil. LC-MS: 557.3 (MH⁺)

¹H NMR (DMSO-d₆, 300 MHz): δ 0.81-1.25 (5H, m), 1.27-2.07 (12H, m), 2.10-2.35 (2H, m), 3.09-3.46 (4H, m), 3.50-3.65 (1H, m). 3.91-4.08 (3H, m), 4.12-4.34 (3H, m), 4.89-5.07 (2H, m), 6.57-6.80 (2H, m), 7.06-7.23 (1H, m), 8.05-8.39 (1H, m).

### (x) Production of (3S,7R,8aR)-2-{(2S)-2-cyclohexyl-2-[(N-methyl-L-alanyl)amino]acetyl}-N-[(4R)-7-fluoro-3,4-dihydro-2H-chromen-4-yl]-7-(2,2,2-trifluoroethoxy)octahydropyrrolo[1,2-a]pyrazine-3-carboxamide

(3S,7R,8aR)-2-[(2S)-2-Amino-2-cyclohexylacetyl]-N-[(4R)-7-fluoro-3,4-dihydro-2H-chromen-4-yl]-7-(2,2,2-trifluoroethoxy)octahydropyrrolo[1,2-a]pyrazine-3-carboxamide (83.5 mg), N-(tert-butoxycarbonyl)-N-methyl-L-alanine (45.7 mg), 0-(benzotriazol-1-yl)-N,N,N′,N′-tetramethyluronium hexafluorophosphate (114 mg) and N,N-diisopropylethylamine (104 μL) were mixed in N,N-dimethylformamide (3 mL), and the mixture was stirred at room temperature for 15 hr. The mixture was diluted with ethyl acetate (10 mL), and washed with water (10 mL) and saturated brine (10 mL). The organic layer was dried over anhydrous magnesium sulfate, and concentrated under reduced pressure. The residue was purified by basic silica gel column chromatography (ethyl acetate/hexane=20/80→100/0) to give a colorless oil. This colorless oil was dissolved in ethyl acetate (4 mL), 4M hydrogen chloride-ethyl acetate solution (4 mL) was added thereto, and the mixture was stirred at room temperature for 2 hr. To the mixture was added water (20 mL), and the aqueous layer was separated. The aqueous layer was neutralized with 1M aqueous sodium hydroxide solution, and the mixture was extracted with ethyl acetate (10 mL), and washed with saturated brine (10 mL). The organic layer was dried over anhydrous magnesium sulfate, and concentrated under reduced pressure. The residue was dissolved in ethyl acetate (1 mL), hexane (3 mL) was added thereto, and the mixture was stirred at room temperature for 15 min. The precipitated solid was collected by filtration, and washed with ethyl acetate-hexane to give the title compound (63.1 mg) as a white solid. LC-MS: 642.3 (MH⁺)

¹H NMR (DMSO-d₆, 300 MHz): δ 0.82-1.27 (8H, m), 1.47-2.11 (13H, m), 2.16 (3H, s), 2.21-2.31 (1H, m), 2.88-3.01 (1H, m), 3.09-3.24 (1H, m), 3.35-3.62 (2H, m), 3.91-4.56 (6H, m), 4.67-4.81 (1H, m), 4.88-5.05 (2H, m), 6.56-6.76 (2H, m), 7.08-7.28 (1H, m), 7.85-8.02 (1H, m), 8.12-8.38 (1H, m).

### Example 34

### Production of (3S,8aR)-2-{(2S)-2-cyclohexyl-2-[(N-methyl-L-alanyl)amino]acetyl}-N-[(1R)-1,2,3,4-tetrahydronaphthalen-1-yl]octahydropyrrolo[1,2-a]pyrazine-3-carboxamide

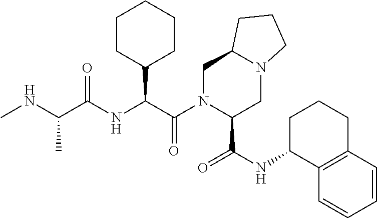

### (i) Production of tert-butyl[(1S)-2-({(1S)-1-cyclohexyl-2-oxo-2-[(3S,8aR)-3-[(1R)-1,2,3,4-tetrahydronaphthalen-1-ylcarbamoyl]hexahydropyrrolo[1,2-a]pyrazin-2(1H)-yl]ethyl}amino)-1-methyl-2-oxoethyl]methylcarbamate

To a solution of methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (2.25 g) obtained in Example 1 (v) in a mixed solvent of tetrahydrofuran/water (3/1, 20 mL) was added lithium hydroxide monohydrate (557 mg), and the mixture was stirred with heating at 50° C. for 2 hr. The reaction mixture was allowed to cool to room temperature, neutralized with 1M hydrochloric acid (13 mL), and concentrated under reduced pressure. The obtained residue was dissolved in tetrahydrofuran (25 mL), (1R)-1,2,3,4-tetrahydronaphthalen-1-amine (0.974 mL), 1-hydroxybenzotriazole (1.19 g) and N-[3-(dimethylamino)propyl]-N′-ethylcarbodiimide hydrochloride (1.69 g) were added thereto, and the reaction mixture was stirred at room temperature for 3 hr. The mixture was diluted with ethyl acetate (500 mL), and washed with water (50 mL) and saturated brine (50 mL). The organic layer was dried over anhydrous magnesium sulfate, filtered, and concentrated under reduced pressure. The residue was subjected to basic silica gel column chromatography (ethyl acetate/hexane=5/95→60/40), and the collected fractions were concentrated to give the title compound (1.80 g) as a colorless amorphous powder. LC-MS: 624 (MH⁺).

### (ii) Production of (3S,8aR)-2-{(2S)-2-cyclohexyl-2-[(N-methyl-L-alanyl)amino]acetyl}-N-[(1R)-1,2,3,4-tetrahydronaphthalen-1-yl]octahydropyrrolo[1,2-a]pyrazine-3-carboxamide

tert-Butyl[(1S)-2-({(1S)-1-cyclohexyl-2-oxo-2-[(3S,8aR)-3-[(1R)-1,2,3,4-tetrahydronaphthalen-1-ylcarbamoyl]hexahydropyrrolo[1,2-a]pyrazin-2(1H)-yl]ethyl}amino)-1-methyl-2-oxoethyl]methylcarbamate (250 mg) was dissolved in 4M hydrogen chloride-ethyl acetate solution (5 mL), and the solution was stirred at room temperature for 30 min. The reaction mixture was concentrated under reduced pressure, and the residue was purified by basic silica gel column chromatography (ethyl acetate) to give the title compound (181 mg) as a colorless amorphous powder. LC-MS: 524.3 (MH⁺).

¹H NMR (DMSO-d₆, 300 MHz): δ 0.79-1.41 (9H, m), 1.46-2.08 (15H, m), 2.09-2.23 (3H, m), 2.55-3.28 (6H, m), 3.29-3.71 (2H, m), 3.77-5.08 (4H, m), 6.89-7.31 (4H, m), 7.64-8.44 (2H, m).

### Example 35

### Production of N-{(1S)-1-cyclohexyl-2-[(3S,8aR)-3-[4-(4-fluoronaphthalen-1-yl)-1,3-thiazol-2-yl]hexahydropyrrolo[1,2-a]pyrazin-2(1H)-yl]-2-oxoethyl}-N-2-methyl-L-alaninamide dihydrochloride

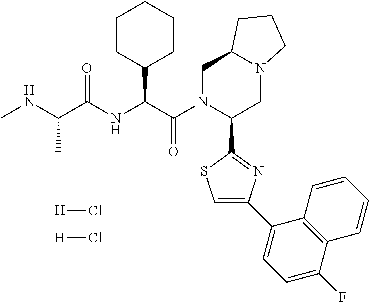

### (i) Production of (3S,8aR)-3-[4-(4-fluoronaphthalen-1-yl)-1,3-thiazol-2-yl]octahydropyrrolo[1,2-a]pyrazine

To a solution of 1-(4-fluoronaphthalen-1-yl)ethanone (500 mg) in acetic acid (5 mL) was added a solution (5 mL) of bromine in acetic acid, and the reaction mixture was stirred at room temperature for 3 hr, and concentrated. The residue was dissolved in 1,2-dimethoxyethane (7.5 mL), tert-butyl(3S,8aR)-3-carbamothioylhexahydropyrrolo[1,2-a]pyrazine-2(1H)-carboxylate (450 mg) and potassium hydrogen carbonate (789 mg) were added thereto, and the mixture was stirred at room temperature for 3 hr. To the mixture were added trifluoroacetic anhydride (1.10 mL) and 2,4,6-collidine (0.333% mL), and the mixture was stirred at room temperature for 2 hr.

The mixture was diluted with ethyl acetate (200 mL), and, washed with water (50 mL) and saturated brine (50 mL). The organic layer was dried over anhydrous magnesium sulfate, filtered, and concentrated under reduced pressure. The residue was subjected to basic silica gel column chromatography (ethyl acetate/hexane=0/100→30/70), and the collected fractions were concentrated to give a colorless amorphous powder (512 mg). This amorphous powder was dissolved in 4M hydrogen chloride-ethyl acetate solution (1 mL), and the solution was stirred at room temperature for 30 min. The mixture was concentrated under reduced pressure, and the residue was dissolved in methanol (5 mL). The solution was filtered through a pad filled with basic silica gel (30 g), and the pad was washed with ethyl acetate (200 mL). The filtrate was concentrated, and the residue was purified by silica gel column chromatography (methanol/ethyl acetate=0/100→10/90) to give the title compound (197 mg) as a colorless amorphous powder. LC-MS: 354.2 (MH⁺).

¹H NMR (DMSO-d₆, 300 MHz): δ 1.13-1.30 (1H, m), 1.53-1.66 (2H, m), 1.67-1.81 (1H, m), 1.82-1.94 (1H, m), 1.97-2.14 (1H, m), 2.42 (1H, dd, J=10.6, 4.0 Hz), 2.58-2.69 (1H, m), 2.88-3.01 (2H, m), 3.04-3.21 (1H, m), 3.67 (1H, J=9.6 Hz, d), 4.34 (1H, d, J=2.6 Hz), 7.41 (1H, J=10.6, 8.1 Hz, dd), 7.59-7.79 (4H, m), 8.08-8.15 (1H, m), 8.42-8.49 (1H, m).

### (ii) Production of N-{(1S)-1-cyclohexyl-2-[(3S,8aR)-3-[4-(4-fluoronaphthalen-1-yl)-1,3-thiazol-2-yl]hexahydropyrrolo[1,2-a]pyrazin-2(1H)-yl]-2-oxoethyl}-N-2-methyl-L-alaninamide dihydrochloride

A solution of (3S,8aR)-3-[4-(4-fluoronaphthalen-1-yl)-1,3-thiazol-2-yl]octahydropyrrolo[1,2-a]pyrazine (173 mg), (2S)-{[N-(tert-butoxycarbonyl)-N-methyl-L-alanyl]amino}(cyclohexyl)ethanoic acid (335 mg) and 4-(4,6-dimethoxy-1,3,5-triazin-2-yl)-4-methylmorpholinium chloride (176 mg) in a mixed solvent of tetrahydrofuran (2 mL)/2-propanol (2 mL) was stirred at room temperature for 3 hr. The mixture was diluted with ethyl acetate (150 mL), and washed with water (25 mL) and saturated brine (25 mL). The organic layer was dried over anhydrous magnesium sulfate, filtered, and concentrated under reduced pressure. The residue was subjected to silica gel column chromatography (ethyl acetate/hexane=95/5→40/60), and the collected fractions were concentrated to give a colorless amorphous powder. This amorphous powder was dissolved in 4M hydrogen chloride-ethyl acetate solution (5 mL), and the solution was stirred at room temperature for 30 min. To the reaction solution was added diethyl ether (20 mL), and the precipitate was collected by filtration, and dried to give the title compound (171 mg) as a colorless amorphous powder. LC-MS: 578.4 (MH⁺).

¹H NMR (DMSO-d₆, 300 MHz): δ 0.87-1.34 (4H, m), 1.36-1.50 (4H, m), 1.55-2.01 (8H, m), 2.01-2.43 (4H, m), 2.60 (1H, t, J=5.1 Hz), 3.01-3.18 (1H, m), 3.22-4.15 (5H, m), 4.29-5.09 (3H, m), 5.72-6.65 (1H, m), 7.35-7.53 (1H, m), 7.59-7.84 (3H, m), 7.89-8.06 (1H, m), 8.08-8.19 (1H, m), 8.25-8.58 (1H, m), 8.76-9.41 (2H, m), 9.65-10.05 (1H, m), 11.66-12.46 (1H, m).

### Example 36

### Production of N-{(1S)-1-cyclohexyl-2-[(3S,8aS)-3-{4-[(4-fluorophenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

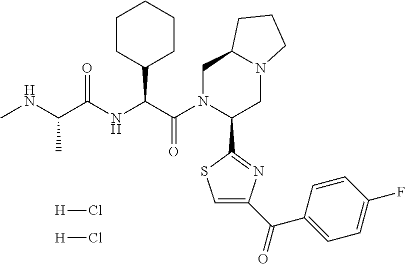

### (i) Production of tert-butyl(3S,8aS)-3-carbamoylhexahydropyrrolo[1,2-a]pyrazine-2(1H)-carboxylate

To a solution of 2-tert-butyl 3-methyl(3S,8aS)-hexahydropyrrolo[1,2-a]pyrazine-2,3(1H)-dicarboxylate (743 mg) in a mixed solvent (10 mL) of tetrahydrofuran/water (4/1) was added lithium hydroxide monohydrate (164 mg), and the mixture was stirred with heating at 50° C. for 3 hr. The reaction mixture was allowed to cool to room temperature, neutralized with 1M hydrochloric acid (3.9 mL), and concentrated under reduced pressure. The obtained residue was dissolved in N,N-dimethylformamide (10 mL), 1-hydroxybenzotriazole ammonium salt (596 mg) and N-[3-(dimethylamino)propyl]-N′-ethylcarbodiimide hydrochloride (751 mg) were added thereto, and the reaction mixture was stirred at room temperature for 3 hr. The solvent was evaporated under reduced pressure, and the obtained residue was diluted with ethyl acetate (500 mL) and water (100 mL). The organic layer was dried over anhydrous magnesium sulfate, and the insoluble material was filtered off. The filtrate was concentrated under reduced pressure, and the residue was purified by basic silica gel column chromatography (ethyl acetate/hexane=10/90→100/0→ethyl acetate/methanol=90/10) to give the title compound (291 mg) as a pale-yellow amorphous powder. LC-MS: 270.2 (MH⁺).

¹H NMR (DMSO-d₆, 300 MHz): δ 1.29-1.42 (1H, m), 1.37 (9H, s), 1.49-1.90 (4H, m), 2.56-2.66 (2H, m), 2.77-2.91 (2H, m), 3.09-3.20 (1H, m), 3.47-3.58 (1H, m), 4.10-4.17 (1H, m), 6.88 (1H, brs), 7.28 (1H, brs).

### (ii) Production of tert-butyl(3S,8aS)-3-carbamothioylhexahydropyrrolo[1,2-a]pyrazine-2(1H)-carboxylate

To a solution of tert-butyl(3S,8aS)-3-carbamoylhexahydropyrrolo[1,2-a]pyrazine-2(1H)-carboxylate (290 mg) in 1,2-dimethoxyethane (5 mL) was added 2,4-bis(4-methoxyphenyl)-1,3,2,4-dithiadiphosphetane-2,4-disulfide (435 mg), and the mixture was heated at 60° C. for 3 hr. The reaction mixture was allowed to cool to room temperature, the solvent was evaporated under reduced pressure, and the obtained residue was purified by basic silica gel column chromatography (ethyl acetate/hexane=5/95→100/0) to give the title compound (125 mg) as a pale-yellow amorphous powder. LC-MS: 286.1 (MH⁺).

¹H NMR (DMSO-d₆, 300 MHz): δ 1.26-1.43 (10H, m), 1.48-1.62 (1H, m), 1.65-1.78 (1H, m), 1.82-1.96 (1H, m), 2.57-2.67 (1H, m), 2.69-2.76 (1H, m), 2.89-3.11 (2H, m), 3.14-3.26 (1H, m), 3.31-3.52 (2H, m), 4.60 (1H, dd, J=8.5, 5.9 Hz), 9.10 (1H, brs), 9.51 (1H, brs).

### (iii) Production of tert-butyl(3S,8aS)-3-[4-(ethoxycarbonyl)-1,3-thiazol-2-yl]hexahydropyrrolo[1,2-a]pyrazine-2(1H)-carboxylate

To a solution of tert-butyl(3S,8aS)-3-carbamothioylhexahydropyrrolo[1,2-a]pyrazine-2(1H)-carboxylate (120 mg) in 1,2-dimethoxyethane (3 mL) were added potassium hydrogen carbonate (336 mg) and ethyl 3-bromo-2-oxopropanoate (0.158 mL), and the mixture was stirred at room temperature for 2 hr. The reaction mixture was cooled to 0° C., trifluoroacetic anhydride (0.292 mL) and 2,4,6-trimethylpyridine (0.089 mL) were added thereto, and the mixture was allowed to warm to room temperature and stirred for 18 hr. The reaction mixture was diluted with ethyl acetate (100 mL), and washed with water (10 mL). The organic layer was dried over anhydrous magnesium sulfate, and the insoluble material was filtered off. The filtrate was concentrated under reduced pressure, and the residue was purified by basic silica gel column chromatography (ethyl acetate/hexane=0/100→100/0) to give the title compound (85 mg) as a colorless oil. LC-MS: 382 (MH⁺).

¹H NMR (DMSO-d₆, 300 MHz): δ 1.26-1.35 (12H, m), 1.37-1.65 (2H, m), 1.71-1.96 (2H, m), 2.55-2.67 (1H, m), 2.69-2.79 (1H, m), 3.06-3.22 (3H, m), 3.35-3.44 (1H, m), 3.62 (1H, dd, J=13.3, 4.1 Hz), 4.29 (2H, q, J=7.0 Hz), 5.22 (1H, t, J=5.0 Hz), 8.41 (1H, s).

### (iv) Production of tert-butyl(3S,6aS)-3-{4-[methoxy(methyl)carbamoyl]-1,3-thiazol-2-yl}hexahydropyrrolo[1,2-a]pyrazine-2(1H)-carboxylate

To a solution of tert-butyl(3S,8aS)-3-[4-(ethoxycarbonyl)-1,3-thiazol-2-yl]hexahydropyrrolo[1,2-a]pyrazine-2(1H)-carboxylate (120 mg) in a mixed solvent (4 mL) of tetrahydrofuran/water (3/1) was added lithium hydroxide monohydrate (19.8 mg), and the mixture was stirred with heating at 50° C. for 2 hr. The reaction mixture was allowed to cool to room temperature, N,O-dimethylhydroxyamine hydrochloride (46.1 mg), 1-hydroxybenzotriazole (71.2 mg) and N-[3-(dimethylamino)propyl]-N′-ethylcarbodiimide hydrochloride (106 mg) were added thereto. The reaction mixture was stirred at room temperature for 2 hr. The mixture was diluted with ethyl acetate (100 mL), and washed with water (10 mL). The organic layer was dried over anhydrous magnesium sulfate, and the insoluble material was filtered off. The filtrate was concentrated under reduced pressure, and the residue was purified by basic silica gel column chromatography (ethyl acetate/hexane=10/90→50/50) to give the title compound (42.4 mg) as a colorless amorphous powder. LC-MS: 397 (MH⁺).

¹H NMR (CDCl₃, 300 MHz): δ 1.39 (9H, s), 1.47-2.12 (4H, m), 2.67-2.79 (1H, m), 2.82-2.93 (1H, m), 3.18-3.34 (3H, m), 3.43 (3H, s), 3.50-3.59 (1H, m), 3.63-3.71 (1H, m), 3.79 (3H, s), 5.36 (1H, t, J=5.7 Hz), 7.96 (1H, s).

### (v) Production of tert-butyl(3S,8aS)-3-{4-[(4-fluorophenyl)carbonyl]-1,3-thiazol-2-yl}hexahydropyrrolo[1,2-a]pyrazine-2(1H)-carboxylate

A solution of tert-butyl(3S,8aS)-3-{4-[methoxy(methyl)carbamoyl]-1,3-thiazol-2-yl}hexahydropyrrolo[1,2-a]pyrazine-2(1H)-carboxylate (40 mg) in tetrahydrofuran (1 mL) was cooled to 0° C., 1.0 M tetrahydrofuran solution (0.503 mL) of bromo(4-fluorophenyl)magnesium was added thereto, and the mixture was allowed to warm to room temperature, and stirred for 1 hr. To the reaction mixture was added saturated aqueous ammonium chloride solution (5 mL), and the mixture was extracted with ethyl acetate (25 mL). The organic layer was dried over anhydrous magnesium sulfate, the insoluble material was filtered off, and the filtrate was concentrated. The residue was subjected to basic silica gel column chromatography (ethyl acetate/hexane=99/1→60/40), and the collected fractions were concentrated to give a colorless amorphous powder. (59 mg). LC-MS: 432 (MH⁺).

¹H NMR (CDCl₃, 300 MHz): δ 1.18-1.31 (1H, m), 1.37-1.44 (9H, m), 1.50-2.09 (4H, m), 2.67-2.82 (1H, m), 2.85-2.97 (1H, m), 3.17-3.40 (2H, m), 3.48-3.60 (1H, m), 3.67-3.78 (1H, m), 5.39 (1H, t, J=5.5 Hz), 7.15 (2H, td, J=8.6, 1.3 Hz), 8.12-8.19 (1H, m), 8.25-8.37 (2H, m).

### (vi) Production of N-{(1S)-1-cyclohexyl-2-[(3S,8aS)-3-{4-[(4-fluorophenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

tert-Butyl(3S,8aS)-3-{4-[(4-fluorophenyl)carbonyl]-1,3-thiazol-2-yl}hexahydropyrrolo[1,2-a]pyrazine-2(1H)-carboxylate (34 mg) was dissolved in about 5-10% hydrogen chloride-methanol solution (3 mL), and the solution was stirred at room temperature for 6 hr, and concentrated under reduced pressure. The residue was dissolved in a mixed solvent (1 mL) of tetrahydrofuran/2-propanol (1/1), (2S)-{[N-(tert-butoxycarbonyl)-N-methyl-L-alanyl]amino}(cyclohexyl)ethanoic acid (34.0 mg), 4-(4,6-dimethoxy-1,3,5-triazin-2-yl)-4-methylmorpholinium chloride (32.6 mg) and 4-methylmorpholine (0.348 mL) were added thereto, and the mixture was stirred at room temperature for 18 hr. The mixture was filtered through a pad filled with basic silica gel, and the pad was washed with ethyl acetate (100 mL). The filtrate was concentrated, and the residue was subjected to silica gel column chromatography (ethyl acetate/hexane=10/90→100/0), and the collected fractions were concentrated to give a colorless amorphous powder. This amorphous powder was dissolved in 4M hydrogen chloride-ethyl acetate solution (1 mL), and the solution was stirred at room temperature for 30 min. The mixture was concentrated under reduced pressure to give the title compound (1 mg) as a colorless oil. LC-MS: 566.3 (MH⁺).

### Example 37

### Production of N-{(1S)-1-cyclohexyl-2-[(3S,8aR)-3-{3-[(3,4-difluorophenyl)carbonyl]-1,2,4-oxadiazol-5-yl}hexahydropyrrolo[1,2-a]pyrazin-2(1H)-yl]-2-oxoethyl}-N-2-Methyl-L-alaninamide dihydrochloride

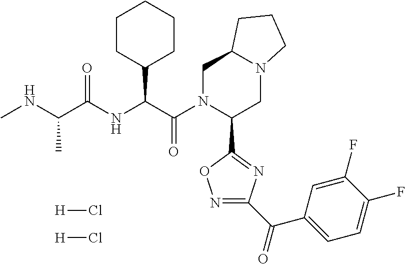

### (i) Production of tert-butyl(3S,8aR)-3-[3-(ethoxycarbonyl)-1,2,4-oxadiazol-5-yl]hexahydropyrrolo[1,2-a]pyrazine-2(1H)-carboxylate

To a solution of 2-tert-butyl 3-methyl(3S,8aR)-hexahydropyrrolo[1,2-a]pyrazine-2,3(1H)-dicarboxylate (2.50 g) in a mixed solvent (25 mL) of tetrahydrofuran/water (4/1) was added lithium hydroxide monohydrate (553 mg), and the mixture was stirred with heating for 50° C. at 3 hr. The reaction mixture was allowed to cool to room temperature, ethyl(2Z)-amino(hydroxyimino)ethanoate (1.74 g), 1-hydroxybenzotriazole (1.19 g) and N-[3-(dimethylamino)propyl]-N′-ethylcarbodiimide hydrochloride (3.37 g) were added thereto, and the mixture was stirred at room temperature for 4 hr. The mixture was diluted with ethyl acetate (300 mL), and washed with water (50 mL). The organic layer was dried over anhydrous magnesium sulfate, filtered, and concentrated under reduced pressure. The residue was dissolved in toluene (25 mL), and the solution was heated under reflux for 12 hr. The reaction mixture was allowed to cool to room temperature, and concentrated. The residue was subjected to silica gel column chromatography (ethyl acetate/hexane=1/99→40/60), and the collected fractions were concentrated to give the title compound (1.88 g) as a colorless oil. LC-MS: 367.2 (MH⁺).

¹H NMR (DMSO-d₆, 300 MHz): δ 1.14-1.26 (1H, m), 1.28-1.49 (12H, m), 1.54-1.70 (2H, m), 1.72-2.12 (3H, m), 2.44-2.55 (1H, m), 2.56-2.85 (1H, m), 2.87-2.98 (1H, m), 3.51-3.63 (1H, m), 3.99-4.11 (1H, m), 4.35-4.45 (2H, m), 5.50-5.68 (1H, m).

### (ii) Production of tert-butyl(3S,8aR)-3-[(3-[methoxy(methyl)carbamoyl]-1,2,4-oxadiazol-5-yl]hexahydropyrrolo[1,2-a]pyrazine-2(1H)-carboxylate

To a solution of tert-butyl(3S,8aR)-3-[3-(ethoxycarbonyl)-1,2,4-oxadiazol-5-yl]hexahydropyrrolo[1,2-a]pyrazine-2(1H)-carboxylate (1.50 g) in a mixed solvent of tetrahydrofuran/water (3/1, 20 mL) was added lithium hydroxide monohydrate (344 mg), and the mixture was stirred at 50° C. for 15 min The reaction mixture was allowed to cool to room temperature, N,O-dimethylhydroxyamine hydrochloride (599 mg), 1-hydroxybenzotriazole (553 mg) and N-[3-(dimethylamino)propyl]-N′-ethylcarbodiimide hydrochloride (1.57 g) were added thereto, and the mixture was stirred at room temperature for 5 hr. The reaction mixture was diluted with ethyl acetate (300 mL), and washed with water (100 mL) and saturated brine (50 mL). The organic layer was dried over anhydrous magnesium sulfate, and the insoluble material was is filtered off. The filtrate was concentrated under reduced pressure, and the residue was subjected to basic silica gel column chromatography (ethyl acetate/hexane=5/95→30/70), and the collected fractions were concentrated to give the title compound (980 mg) as a colorless amorphous powder. LC-MS: 382.2 (MH⁺).

¹H NMR (DMSO-d₆, 300 MHz): δ 1.13-1.30 (1H, m), 1.31-1.47 (9H, m), 1.56-1.72 (2H, m), 1.73-1.97 (2H, m), 2.00-2.11 (1H, m), 2.46-2.56 (1H, m), 2.61-2.87 (1H, m), 2.89-3.01 (1H, m), 3.31 (3H, brs), 3.50-3.61 (1H, m), 3.70 (3H, s), 3.93-4.11 (1H, m), 5.48-5.66 (1H, m).

### (iii) Production of (3,4-difluorophenyl){5-[(3S,8aR)-octahydropyrrolo[1,2-a]pyrazin-3-yl]-1,2,4-oxadiazol-3-yl}methanone

A solution of tert-butyl(3S,8aR)-3-{3-[methoxy(methyl)carbamoyl]-1,2,4-oxadiazol-5-yl}hexahydropyrrolo[1,2-a]pyrazine-2(1H)-carboxylate (500 mg) in tetrahydrofuran (5 mL) was cooled to 0° C., 0.5M tetrahydrofuran solution (13.1 mL) of 3,4-difluorophenylmagnesium bromide was added thereto, and the mixture was allowed to warm to room temperature and stirred for 30 min. To the reaction mixture was added 4M hydrogen chloride-ethyl acetate solution (20 mL), and the mixture was stirred at room temperature for 18 hr. To the mixture was added water (200 mL), and the mixture was washed with ethyl acetate (200 mL). 1M aqueous sodium hydroxide solution was added to the aqueous layer to adjust the pH to about 12, and the mixture was extracted with ethyl acetate (200 mL×2). The combined organic layers were dried over anhydrous magnesium sulfate, and the insoluble material was filtered off. The filtrate was concentrated under reduced pressure, the residue was subjected to silica gel column chromatography (ethyl acetate/hexane=10/90→100/0), and the collected fractions were concentrated to give the title compound (123 mg) as a pale-yellow oil. LC-MS: 335.1 (MH⁺).

¹H NMR (CDCl₃, 300 MHz): δ 1.15-1.40 (1H, m), 1.53-1.89 (4H, m), 1.97-2.23 (2H, m), 2.69 (1H, dd, J=11.3, 3.6 Hz), 2.87 (1H, t, J=10.7 Hz), 3.03 (1H, td, J=8.5, 2.5 Hz), 3.11 (1H, dd, J=11.4, 2.7 Hz), 3.75 (1H, d, J=11.1 Hz), 4.42 (1H, brs), 7.28-7.40 (1H, m), 8.10-8.34 (2H, m).

### (iv) Production of N-{(1S)-1-cyclohexyl-2-[(3S,8aR)-3-{3-[(3,4-difluorophenyl)carbonyl]-1,2,4-oxadiazol-5-yl}hexahydropyrrolo[1,2-a]pyrazin-2(1H)-yl]-2-oxoethyl}-N-2-methyl-L-alaninamide dihydrochloride

To a solution of (3,4-difluorophenyl){5-[(3S,8aR)-octahydropyrrolo[1,2-a]pyrazin-3-yl]-1,2,4-oxadiazol-3-yl}methanone (120 mg), (2S)-[(tert-butoxycarbonyl)amino](cyclohexyl)ethanoic acid (204 mg) and 1-hydroxybenzotriazole (53.4 mg) in a mixed solvent of tetrahydrofuran (4 mL) and N,N-dimethylformamide (2 mL) was added N-[3-(dimethylamino)propyl]-N′-ethylcarbodiimide hydrochloride (106 mg), and the reaction mixture was stirred at room temperature for 3 hr. To the mixture was added O-(1H-6-chlorobenzotriazol-1-yl)-N,N,N′,N′-tetramethyluronium hexafluorophosphate (297 mg), and the mixture was stirred at room temperature for 72 hr. The reaction mixture was diluted with ethyl acetate (100 mL), and washed with water (20 mL). The organic layer was dried over anhydrous magnesium sulfate, filtered, and concentrated under reduced pressure. The residue was dissolved in 2M hydrogen chloride-ethyl acetate solution (3 mL), and the mixture was stirred at room temperature for 30 min. To the mixture was added water (100 mL), and the mixture was washed with ethyl acetate (50 mL). 1M aqueous sodium hydroxide solution was added to the aqueous layer to adjust the pH to about 12, and the mixture was extracted with ethyl acetate (100 mL×2). The combined organic layers were dried over anhydrous magnesium sulfate, the insoluble material was filtered off, and the filtrate was concentrated under reduced pressure. The residue was dissolved in N,N-dimethylformamide (5 mL), O-(1H-6-chlorobenzotriazol-1-yl)-N,N,N′,N′-tetramethyluronium hexafluorophosphate (280 mg), N-ethyldiisopropylamine (0.147 mL) and N-(tert-butoxycarbonyl)-N-methyl-L-alanine (160 mg) were added thereto, and the reaction mixture was stirred at room temperature for 2 hr. The mixture was diluted with ethyl acetate (100 mL), and washed with saturated brine (20 mL). The organic layer was dried over anhydrous magnesium sulfate, filtered, and concentrated under reduced pressure. The residue was subjected to silica gel column chromatography (ethyl acetate/hexane=5/95→60/40), and the collected fractions were concentrated to give a pale-yellow amorphous powder. This amorphous powder was dissolved in 4M hydrogen chloride-ethyl acetate solution (5 mL), and the solution was stirred at room temperature for 30 min. To the reaction solution was'added diethyl ether (25 mL), and the precipitate was collected by filtration, and dried to give the title compound (103 mg) as a colorless amorphous powder. LC-MS: 559.3 (MH⁺).

¹H NMR (DMSO-d₆, 300 MHz): δ 0.92-1.22 (6H, m), 1.37 (3H, d, J=6.6 Hz), 1.48-2.21 (8H, m), 2.31-2.37 (1H, m), 2.47 (3H, brs), 2.94-4.33 (7H, m), 4.53-5.11 (2H, m), 5.63-6.86 (1H, m), 7.33-8.45 (3H, m), 8.61-9.05 (2H, m), 9.23-9.77 (1H, m), 11.73-12.68 (1H, m).

### Example 38

### Production of (3S,8aR)-2-[(2S)-2-cyclohexyl-2-{[(2S)-2-(methylamino)butanoyl]amino]acetyl}-N-[(4R)-3,4-dihydro-2H-chromen-4-yl]octahydropyrrolo[1,2-a]pyrazine-3-carboxamide dihydrochloride

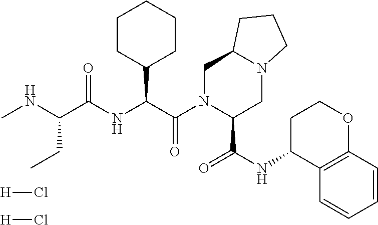

### (i) Production of (3S,8aR)—N-[(4R)-3,4-dihydro-2H-chromen-4-yl]octahydropyrrolo[1,2-a]pyrazine-3-carboxamide dihydrochloride

To a solution of 2-tert-butyl 3-methyl(3S,8aR)-hexahydropyrrolo[1,2-a]pyrazine-2,3(1H)-dicarboxylate (557 mg) in a mixed solvent (10 mL) of tetrahydrofuran/water (4/1) was added lithium hydroxide monohydrate (123 mg), and the mixture was stirred with heating at 50° C. for 4 hr, and concentrated under reduced pressure. The obtained residue was dissolved in N,N-dimethylformamide (5 mL), (4R)-3,4-dihydro-2H-chromen-4-amine hydrochloride (400 mg), N-ethyldiisopropylamine (0.853 mL) and O-(1H-6-chlorobenzotriazol-1-yl)-N,N,N′,N′-tetramethyluronium hexafluorophosphate (1.62 g) were added thereto, and the reaction mixture was stirred at room temperature for 18 hr. The mixture was diluted with ethyl acetate (250 mL), and washed with water (100 mL). The organic layer was dried over anhydrous magnesium sulfate, the insoluble material was filtered off, and the filtrate was concentrated. The residue was subjected to silica gel column chromatography (ethyl acetate/methanol=100/0→90/10), and the collected fractions were concentrated to give a colorless amorphous powder (290 mg). This amorphous powder was dissolved in 4M hydrogen chloride-ethyl acetate solution (5 mL), and the solution was stirred at room temperature for 1 hr, and concentrated. The residue was dissolved in methanol (5 mL), diethyl ether (20 mL) was added thereto, and the precipitate was collected by filtration, and dried under reduced pressure to give the title compound (168 mg) as a colorless amorphous powder. LC-MS: 302.2 (MH⁺).

¹H NMR (DMSO-d₆, 300 MHz): δ 1.80-2.19 (6H, m), 3.05-3.96 (8H, m), 4.10-4.42 (3H, m), 6.81 (1H, d, J=7.9 Hz), 6.90 (1H, t, J=7.4 Hz), 7.14-7.29 (2H, m), 9.39 (2H, brs), 12.38 (1H, brs).

### (ii) Production of (3S,8aR)-2-[(2S)-2-amino-2-cyclohexylacetyl]-N-[(4R)-3,4-dihydro-2H-chromen-4-yl]octahydropyrrolo[1,2-a]pyrazine-3-carboxamide

To a solution of ((2S)-[(tert-butoxycarbonyl)amino] (cyclohexyl)ethanoic acid (121 mg) and O-(7-azabenzotriazol-1-yl)-N,N,N′,N′-tetramethyluronium hexafluorophosphate (325 mg) in N,N-dimethylformamide (2 mL) was added a solution (1 mL) of (3S,8aR)—N-[(4R)-3,4-dihydro-2H-chromen-4-yl]octahydropyrrolo[1,2-a]pyrazine-3-carboxamide dihydrochloride (160 mg) and N-ethyldiisopropylamine (0.155 mL) in N,N-dimethylformamide. The reaction mixture was stirred at room temperature for 3 hr, diluted with ethyl acetate (300 mL), and washed with water (50 mL). The organic layer was washed with saturated brine (50 mL), and dried over anhydrous magnesium sulfate, the insoluble material was filtered off, and the filtrate was concentrated. The residue was dissolved in 4M hydrogen chloride-ethyl acetate solution (2 mL), and the mixture was stirred at room temperature for 2 hr. To the reaction mixture were added saturated sodium hydrogen carbonate solution (40 mL) and ethyl acetate (150 mL), the organic layer was dried over anhydrous magnesium sulfate. The insoluble material was filtered off, and the filtrate was concentrated. Without further purification, the obtained residue (300 mg) was used for the next reaction. LC-MS: 441.3 (MH⁺).

### (iii) Production of (3S,8aR)-2-[(2S)-2-cyclohexyl-2-{[(2S)-2-(methylamino)butanoyl]amino}acetyl]-N-[(4R)-3,4-dihydro-2H-chromen-4-yl]octahydropyrrolo[1,2-a]pyrazine-3-carboxamide dihydrochloride

To a solution of the crude product (300 mg) of (3S,8aR)-2-[(2S)-2-amino-2-cyclohexylacetyl]-N-[(4R)-3,4-dihydro-2H-chromen-4-yl]octahydropyrrolo[1,2-a]pyrazine-3-carboxamide and N-ethyldiisopropylamine (0.155 mL) in N,N-dimethylformamide (1 mL) was added a solution (2 mL) of (2S)-2-[(tert-butoxycarbonyl)(methyl)amino]butanoic acid (111 mg) and O-(7-azabenzotriazol-1-yl)-N,N,N′,N′-tetramethyluronium hexafluorophosphate (325 mg) in N,N-dimethylformamide, and the reaction mixture was stirred at room temperature for 18 hr. The mixture was diluted with ethyl acetate (250 mL), and washed with water (100 mL). The organic layer was washed with saturated brine, and dried over anhydrous magnesium sulfate. The insoluble material was filtered off, and the filtrate was concentrated. The residue was subjected to silica gel column chromatography (ethyl acetate/hexane=10/90→100/0), and the collected fractions were concentrated to give a colorless amorphous powder. The obtained amorphous powder was dissolved in acetonitrile (3 mL), and the solution was purified by preparative HPLC

[Apparatus: Gilson, Inc. high throughput purification system, Column: CombiPrep ProC18RS (50×20 mm S-5 μm), Solvent: SOLUTION A; 0.1% trifluoroacetic acid-containing water, SOLUTION B; 0.1% trifluoroacetic acid containing acetonitrile, Gradient cycle: 0.00 min (SOLUTION A/SOLUTION B=99/1), 1.25 min (SOLUTION A/SOLUTION B=99/1), 6.50 min (SOLUTION A/SOLUTION B=0/100), 7.30 min(SOLUTION A/SOLUTION B=0/100), 7.40 min (SOLUTION A/SOLUTION B=95/5), Flow rate: 25 mL/min, Detection method: UV 220 nm]. The residue was diluted with ethyl acetate (200 mL) and saturated aqueous sodium hydrogen carbonate solution (50 mL). The organic layer was dried over anhydrous magnesium sulfate, the insoluble material was filtered off, and the filtrate was concentrated. The residue was dissolved in 4M hydrogen chloride-cyclopentyl methyl ether solution (3 mL), and the solution was stirred at room temperature for 1 hr. The precipitate was collected by filtration, and dried under reduced pressure to give the title compound (17 mg) as a colorless amorphous powder. LC-MS: 540.4 (MH⁺).

### Example 39

### Production of N-{(1S)-1-cyclohexyl-2-[(3S,8aR)-3-{4-[(4-fluorophenyl)carbonyl]-1,3-oxazol-2-yl}hexahydropyrrolo[1,2-a]pyrazin-2(1H)-yl]-2-oxoethyl}-N-2-methyl-L-alaninamide dihydrochloride

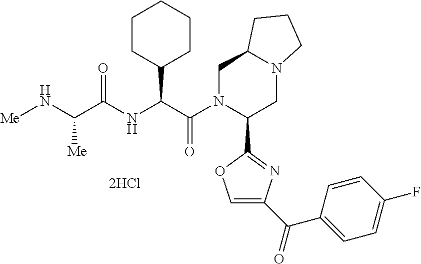

### (i) Production of tert-butyl(3S,8aR)-3-{[1-(hydroxymethyl)-2-methoxy-2-oxoethyl]carbamoyl}hexahydropyrrolo[1,2-a]pyrazine-2(1H)-carboxylate

To a solution of 2-tert-butyl 3-methyl(3S,8aR)-hexahydropyrrolo[1,2-a]pyrazine-2,3(1H)-dicarboxylate (1.15 g) in a mixed solvent (20 mL) of tetrahydrofuran/water (4/1) was added lithium hydroxide monohydrate (270 mg), and the mixture was stirred with heating at 60° C. for 4 hr. The reaction mixture was allowed to cool to room temperature, and neutralized with 1M hydrochloric acid (6 mL), and the collected fractions were concentrated under reduced pressure. The residue was dissolved in N,N-dimethylformamide (20 mL), methyl serinate hydrochloride (780 mg), N-ethyldiisopropylamine (1.28 mL), 1-hydroxybenzotriazole (760 mg) and N-[3-(dimethylamino)propyl]-N′-ethylcarbodiimide hydrochloride (960 mg) were added thereto, and the mixture was stirred at room temperature for 7 hr. The solvent was evaporated under reduced pressure, and the residue was diluted with ethyl acetate (30 mL) and saturated aqueous sodium hydrogen carbonate solution (30 mL). The aqueous layer was extracted with ethyl acetate (30 mL), and the combined organic layers were dried over anhydrous magnesium sulfate. The insoluble material was filtered off. The filtrate was concentrated under reduced pressure, and the residue was purified by silica gel column chromatography (ethyl acetate/hexane=30/70→100/0→ethyl acetate/methanol=80/20) to give the title compound (1.36 g) as a pale-yellow solid. LC-MS: 372.3 (MH⁺).

### (ii) Production of tert-butyl(3S,8aR)-3-[4-(methoxycarbonyl)-4,5-dihydro-1,3-oxazol-2-yl]hexahydropyrrolo[1,2-a]pyrazine-2(1H)-carboxylate

To a solution of tert-butyl(3S,8aR)-3-{[1-(hydroxymethyl)-2-methoxy-2-oxoethyl]carbamoyl}hexahydropyrrolo[1,2-a]pyrazine-2(1H)-carboxylate (1.36 g) in tetrahydrofuran (20 mL) was added (methoxycarbonyl)[(triethylammonio)sulfonyl]methanide (1.19 g), and the mixture was stirred with heating at 60° C. for 12 hr. The reaction mixture was allowed to cool to room temperature, and diluted with water (40 mL) and ethyl acetate (40 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by basic silica gel column chromatography (ethyl acetate/hexane=5/95→100/0) and silica gel column chromatography (ethyl acetate/hexane=20/60→100/0) to give the title compound (338 mg) as a pale-yellow solid. LC-MS: 354.2 (MH⁺).

### (iii) Production of tert-butyl(3S,8aR)-3-[4-(methoxycarbonyl)-1,3-oxazol-2-yl]hexahydropyrrolo[1,2-a]pyrazine-2(1H)-carboxylate

To a solution of tert-butyl(3S,8aR)-3-[4-(methoxycarbonyl)-4,5-dihydro-1,3-oxazol-2-yl]hexahydropyrrolo[1,2-a]pyrazine-2(1H)-carboxylate (338 mg) in (trifluoromethyl)benzene (10 mL) were added 1,8-diazabicyclo[5.4.0]undec-7-ene (0.18 mL) and bromo(trichloro)methane (0.36 mL), and the mixture was stirred at room temperature for 60 hr. The reaction mixture was diluted with water (50 mL) and ethyl acetate (50 mL), and the aqueous layer was extracted with ethyl acetate (50 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (219 mg) as a colorless oil. LC-MS: 352.2 (MH⁺).

¹H NMR (DMSO-d₆, 300 MHz): δ 1.21-1.27 (1H, m), 1.33-1.44 (9H, m), 1.55-1.68 (2H, m), 1.72-1.93 (2H, m), 1.96-2.09 (1H, m), 2.35-2.46 (1H, m), 2.66-2.87 (1H, m), 2.88-2.97 (1H, m), 3.46-3.57 (1H, m), 3.80 (3H, s), 3.94-4.10 (1H, m), 5.24-5.36 (1H, m), 8.81-8.83 (1H, m).

### (iv) Production of tert-butyl(3S,8aR)-3-{4-[methoxy(methyl)carbamoyl]-1,3-oxazol-2-yl}hexahydropyrrolo[1,2-a]pyrazine-2(1H)-carboxylate

To a solution of tert-butyl(3S,8aR)-3-[4-(methoxycarbonyl)-1,3-oxazol-2-yl]hexahydropyrrolo[1,2-a]pyrazine-2(1H)-carboxylate (385 mg) in a mixed solvent (6 mL) of tetrahydrofuran/water (4/1) was added lithium hydroxide monohydrate (63.0 mg), and the mixture was stirred with heating at 60° C. for 2 hr. The reaction mixture was cooled to 0° C., and neutralized with 1 M hydrochloric acid (1.4 mL), and the solvent was evaporated under reduced pressure. The obtained residue was dissolved in N,N-dimethylformamide (11 mL), triethylamine (0.46 mL), N,O-dimethylhydroxyamine hydrochloride (322 mg), 1-hydroxybenzotriazole (223 mg) and N-[3-(dimethylamino)propyl]-N′-ethylcarbodiimide hydrochloride (316 mg) were added thereto, and the mixture was stirred at room temperature for 15 hr. The solvent was evaporated under reduced pressure, and the residue was diluted with ethyl acetate (50 mL) and saturated aqueous sodium hydrogen carbonate solution (50 mL). The aqueous layer was extracted with ethyl acetate (50 mL), the combined organic layers were dried over anhydrous magnesium sulfate, and the insoluble material was filtered off. The filtrate was concentrated under reduced pressure, and the residue was purified by silica gel column chromatography (ethyl acetate/hexane=30/70→100/0→ethyl acetate/methanol=80/20) to give the title compound (303 mg) as a pale-yellow amorphous powder. LC-MS: 381.3 (MH⁺).

¹H NMR (DMSO-d₆, 300 MHz): δ 1.21-1.29 (1H, m), 1.33-1.45 (9H, m), 1.55-1.68 (2H, m), 1.72-1.93 (2H, m), 2.00-2.10 (1H, m), 2.41 (1H, dt, J=11.4, 4.2 Hz), 2.64-2.99 (2H, m), 3.32 (3H, s), 3.53 (1H, d, J=9.8 Hz), 3.71 (3H, s), 3.97-4.09 (1H, m), 5.16-5.42 (1H, m), 8.57-8.58 (1H, m).

### (v) Production of tert-butyl(3S,8aR)-3-{4-[(4-fluorophenyl)carbonyl]-1,3-oxazol-2-yl}hexahydropyrrolo[1,2-a]pyrazine-2(1H)-carboxylate

To a solution of tert-butyl(3S,8aR)-3-{4-[methoxy(methyl)carbamoyl]-1,3-oxazol-2-yl}hexahydropyrrolo[1,2-a]pyrazine-2(1H)-carboxylate (300 mg) in tetrahydrofuran (8 mL) was added 1.0M tetrahydrofuran solution (1.6 mL) of bromo(4-fluorophenyl)magnesium under ice-cooling, and the mixture was allowed to warm to room temperature and stirred for 24 hr. The reaction mixture was diluted with saturated aqueous ammonium chloride solution (50 mL) and ethyl acetate (50 mL), and the aqueous layer was extracted with ethyl acetate (50 mL). The combined organic layers were dried over anhydrous magnesium sulfate, and the insoluble material was filtered off. The filtrate was concentrated under reduced pressure, and the residue was purified by silica gel column chromatography (ethyl acetate/hexane-5/95→100/0) to give the title compound (215 mg) as a pale-yellow amorphous powder. LC-MS: 416.2 (MH⁺).

¹H NMR (DMSO-d₆, 300 MHz): δ 1.21-1.28 (1H, m), 1.35-1.45 (9H, m), 1.54-1.70 (2H, m), 1.72-1.96 (2H, m), 2.00-2.11 (1H, m), 2.41-2.47 (1H, m), 2.66-2.99 (2H, m), 3.52-3.62 (1H, m), 3.96-4.09 (1H, m), 5.28-5.44 (1H, m), 7.38 (2H, td, J=8.8, 2.0 Hz), 8.22 (2H, dd, J=8.8, 5.7 Hz), 8.88-8.90 (1H, m).

### (vi) Production of (4-fluorophenyl){2-[(3S,8aR)-octahydropyrrolo[1,2-a]pyrazin-3-yl]-1,3-oxazol-4-yl}methanone

To a solution of tert-butyl(3S,8aR)-3-{4-[(4-fluorophenyl)carbonyl]-1,3-oxazol-2-yl}hexahydropyrrolo[1,2-a]pyrazine-2(1H)-carboxylate (212 mg) in ethyl acetate (2 mL) was added 4M hydrogen chloride-ethyl acetate solution (2 mL), and the mixture was stirred at room temperature for 5 hr. The solvent was evaporated under reduced pressure, and the residue was dissolved in ethyl acetate (40 mL). The solution was washed with saturated aqueous sodium hydrogen carbonate solution (40 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (62 mg) as a pale-yellow amorphous powder. LC-MS: 316.1 (MH⁺).

### (vii) Production of tert-butyl[(1S)-2-({(1S)-1-cyclohexyl-2-[(3S,8aR)-3-{4-[(4-fluorophenyl)carbonyl]-1,3-oxazol-2-yl}hexahydropyrrolo[1,2-a]pyrazin-2(1H)-yl]-2-oxoethyl}amino)-1-methyl-2-oxoethyl]methylcarbamate

To a solution of (4-fluorophenyl){2-[(3S,8aR)-octahydropyrrolo[1,2-a]pyrazin-3-yl]-1,3-oxazol-4-yl}methanone (62 mg) in tetrahydrofuran (2 mL) were added (2S)-{[N-(tert-butoxycarbonyl)-N-methyl-L-alanyl]amino}(cyclohexyl)ethanoic acid (103 mg) and 4-(4,6-dimethoxy-1,3,5-triazin-2-yl)-4-methylmorpholinium chloride (89 mg), and the mixture was stirred at room temperature for 5 hr. To the reaction mixture were added (2S)-{[N-(tert-butoxycarbonyl)-N-methyl-L-alanyl]amino}(cyclohexyl)ethanoic acid (31 mg) and 4-(4,6-dimethoxy-1,3,5-triazin-2-yl)-4-methylmorpholinium chloride (50 mg), and the mixture was stirred at room temperature for 3 hr. The reaction mixture was diluted with ethyl acetate (30 mL), and washed with water (30 mL), and the aqueous layer was extracted with ethyl acetate (30 mL). The combined organic layers were dried over anhydrous magnesium sulfate, and the insoluble material was filtered off. The filtrate was concentrated under reduced pressure, and the residue was purified by preparative HPLC [Apparatus: Gilson, Inc. high throughput purification system, Column: CombiPrepPro C18RS (50×20 mm S-5 μm), Solvent: SOLUTION A; 0.1% trifluoroacetic acid containing water, SOLUTION B; 0.1% trifluoroacetic acid containing acetonitrile, Gradient cycle: 0.00 min (SOLUTION A/SOLUTION B=80/20), 1.20 min (SOLUTION A/SOLUTION B=80/20), 4.75 min (SOLUTION A/SOLUTION B=0/100), 7.30 min(SOLUTION A/SOLUTION B=0/100), 7.40 min (SOLUTION A/SOLUTION B=80/20), Flow rate: 25 mL/min, Detection method: UV 220 nm]. The fraction containing the title compound was diluted with ethyl acetate (50 mL). The solution was washed with saturated aqueous sodium hydrogen carbonate solution (50 mL), and the aqueous layer was extracted with ethyl acetate (50 mL). The combined organic layers were dried over anhydrous magnesium sulfate, and the insoluble material was filtered off. The filtrate was concentrated under reduced pressure to give the title compound (8.3 mg) as a colorless amorphous powder. LC-MS: 640.4 (MH⁺).

¹H NMR (CDCl₃, 300 MHz): δ 0.79-1.17 (7H, m), 1.21-1.29 (5H, m), 1.31-1.37 (2H, m), 1.41-1.52 (7H, m), 1.55-1.96 (8H, m), 2.10-2.21 (1H, m), 2.45-2.63 (1H, m), 2.67-2.91 (3H, m), 2.97-3.17 (1H, m), 3.20-3.45 (1H, m), 3.74 (1H, d, J=10.4 Hz), 4.02-4.28 (1H, m), 4.57-4.84 (1H, m), 4.89-5.07 (1H, m), 5.81-6.07 (1H, m), 7.08-7.23 (2H, m), 8.18-8.47 (3H, m).

### (viii) Production of N-{(1S)-1-cyclohexyl-2-[(3S,8aR)-3-{4-[(4-fluorophenyl)carbonyl]-1,3-oxazol-2-yl}hexahydropyrrolo[1,2-a]pyrazin-2(1H)-yl]-2-oxoethyl}-N-2-methyl-L-alaninamide dihydrochloride

tert-Butyl[(1S)-2-({(1S)-1-cyclohexyl-2-[(3S,8aR)-3-[(4-[(4-fluorophenyl)carbonyl]-1,3-oxazol-2-yl]hexahydropyrrolo[1,2-a]pyrazin-2(1H)-yl]-2-oxoethyl}amino)-1-methyl-2-oxoethyl]methylcarbamate (8.3 mg) was dissolved in 5-10% hydrogen chloride-methanol solution (1 mL), and the solution was stirred at room temperature for 3 hr. The solvent was evaporated under reduced pressure to give the title compound (6.0 mg) as a colorless amorphous powder. LC-MS: 540.3 (MH⁺).

### Example 40

### Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxyoctahydropyrrolo[1,2-a]pyrazine-3-carboxamide dihydrochloride

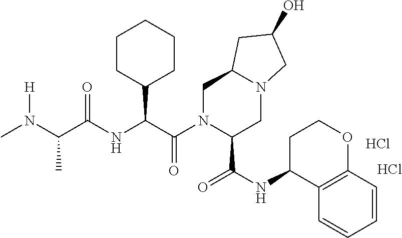

### (i) Production of tert-butyl(2R,4R)-2-(benzylcarbamoyl)-4-hydroxypyrrolidine-1-carboxylate

To a solution of (4R)-1-(tert-butoxycarbonyl)-4-hydroxy-D-proline (50.0 g) in acetonitrile (750 mL) were added benzylamine (28.3 mL), N,N-dimethylpyridin-4-amine (2.64 g), 1-hydroxybenzotriazole (38.0 g) and N-[3-(dimethylamino)propyl]-N′-ethylcarbodiimide hydrochloride (54.0 g), and the mixture was stirred at room temperature for 14 hr. To the reaction mixture was added 5% aqueous citric acid solution (750 mL), and the mixture was extracted with ethyl acetate (1.5 L). The organic layer was washed with saturated aqueous sodium hydrogen carbonate solution (750 mL) and saturated brine (500 mL), and dried over anhydrous magnesium sulfate, and the insoluble material was filtered off. The filtrate was concentrated under reduced pressure, and the residue was charged on a pad filled with basic silica gel, and eluted with ethyl acetate (4.0 L). The fraction containing the title compound was concentrated under reduced pressure to give the title compound (62.3 g) as a colorless solid. LC-MS: 321.2 (MH⁺).

¹H NMR (DMSO-d₆, 300 MHz): δ 1.22-1.47 (9H, m), 1.69-1.82 (1H, m), 2.24-2.41 (1H, m), 3.15-3.26 (1H, m), 3.49 (1H, dd, J=10.7, 5.4 Hz), 4.07-4.42 (4H, m), 5.18-5.34 (1H, m), 7.18-7.37 (5H, m), 8.48 (1H, t, J=6.0 Hz).

### (ii) Production of (4R)—N-benzyl-4-hydroxy-D-prolinamide

tert-Butyl(2R,4R)-2-(benzylcarbamoyl)-4-hydroxypyrrolidine-1-carboxylate (61.0 g) was dissolved in 4M hydrogen chloride-ethyl acetate solution (500 mL), and the solution was stirred at room temperature for 2 hr. The solvent was evaporated under reduced pressure, and the residue was charged on a pad filled with Amberlyst A-21, and eluted with methanol (1.0 L). The fraction containing the title compound was concentrated under reduced pressure to give the title compound (42.8 g) as a colorless solid. LC-MS: 221.1 (MH⁺).

¹H NMR (DMSO-d₆, 300 MHz): δ 1.78 (1H, ddd, J=13.2, 5.9, 4.8 Hz), 2.30 (1H, ddd, J=13.2, 9.4, 5.8 Hz), 2.87 (1H, dd, J=11.2, 3.1 Hz), 3.04 (1H, dd, J=11.2, 5.1 Hz), 3.17 (1H, s), 3.86 (1H, dd, J=9.4, 5.9 Hz), 4.17-4.28 (1H, m), 4.32 (2H, d, J=5.9 Hz), 5.00 (1H, br s), 7.16-7.40 (5H, m), 8.69 (1H, t, J=5.9 Hz).

### (iii) Production of (3R,5R)-5-[(benzylamino)methyl]pyrrolidin-3-al

To a suspension of lithium aluminum hydride (12.9 g) in tetrahydrofuran (300 mL) was added dropwise a suspension of (4R)—N-benzyl-4-hydroxy-D-prolinamide (37.4 g) in tetrahydrofuran under ice-cooling, and the mixture was heated under reflux at 80° C. for 14 hr. The reaction mixture was cooled to 0° C., water (13 mL), 1N aqueous sodium hydroxide solution (13 mL) and water (26 mL) were added thereto, and the insoluble material was filtered off. The filtrate was concentrated under reduced pressure to give the title compound (30.0 g) as a yellow oil. Without further purification, the compound was used for the next reaction. LC-MS: 207.1 (MH⁺).

### (iv) Production of methyl(3S,7R,8aR)-2-benzyl-7-hydroxyoctahydropyrrolo[1,2-a]pyrazine-3-carboxylate

To a solution of (3R,5R)-5-[(benzylamino)methyl]pyrrolidine-3-ol (33.0 g) in toluene (330 mL) were added triethylamine (60 mL) and methyl 2,3-dibromopropanoate (18.2 mL), and the mixture was stirred with heating at 80° C. for 20 hr. The reaction mixture was allowed to cool to room temperature, and diluted with ethyl acetate (500 mL) and water (300 mL), and the aqueous layer was extracted with ethyl acetate (500 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (12.6 g) as an orange oil. LC-MS: 291.2 (MH⁺).

¹H NMR (DMSO-d₆, 300 MHz): δ 1.06-1.21 (1H, m), 1.79-1.94 (1H, m), 1.99-2.14 (2H, m), 2.24 (1H, dd, J=10.6, 4.0 Hz), 2.59 (1H, dd, J=10.7, 3.0 Hz), 2.74 (1H, d, J=9.8 Hz), 2.79-2.89 (1H, m), 3.24 (1H, dd, J=10.7, 1.7 Hz), 3.49-3.56 (1H, m), 3.64 (3H, s), 3.89 (2H, s), 4.14 (1H, dt J=6.8, 3.1 Hz), 4.70 (1H, d, J=4.5 Hz), 7.18-7.38 (5H, m).

### (v) Production of 2-tert-butyl 3-methyl(3S,7R,8aR)-7-hydroxyhexahydropyrrolo[1,2-a]pyrazine-2,3(1H)-dicarboxylate

To a solution of methyl(3S,7R,8aR)-2-benzyl-7-hydroxyoctahydropyrrolo[1,2-a]pyrazine-3-carboxylate (12.0 g) in 5-10% hydrogen chloride-methanol (120 mL) was added palladium-carbon (1.2 g, 50%, wet), and the mixture was stirred at room temperature for 3 hr under a hydrogen atmosphere. The insoluble material was filtered off through a celite pad, and the celite pad was washed with methanol (300 mL). The filtrate and the washing solution were concentrated. The obtained residue was dissolved in tetrahydrofuran (180 mL), saturated aqueous sodium hydrogen carbonate solution (180 mL) and di-tert-butyl dicarbonate (9.93 g) were added thereto, and the mixture was stirred at room temperature for 18 hr. The aqueous layer was extracted with ethyl acetate (300 mL) and a mixed solvent (300 mL) of ethyl acetate/tetrahydrofuran (3/1). The combined organic layers were dried over anhydrous magnesium sulfate, and the insoluble material was filtered off. The filtrate was concentrated under reduced pressure, and the residue was purified by silica gel column chromatography (methanol/ethyl acetate-0/100→80/20) to give the title compound (10.7 g) as a colorless solid. LC-MS: 301.2 (MH⁺).

¹H NMR (DMSO-d₆, 300 MHz): δ 1.08-1.24 (1H, m), 1.35-1.41 (9H, m), 1.70-1.86 (1H, m), 2.09-2.23 (3H, m), 2.57-2.89 (2H, m), 3.30-3.39 (1H, m), 3.67-3.69 (3H, m), 3.79-3.92 (1H, m), 4.11-4.22 (1H, m), 4.53-4.66 (1H, m), 4.77 (1H, d, J=4.3 Hz).

### (vi) Production of methyl(3S,7R,8aR)-7-hydroxyoctahydropyrrolo[1,2-a]pyrazine-3-carboxylate dihydrochloride

2-tert-Butyl 3-methyl(3S,7R,8aR)-7-hydroxyhexahydropyrrolo[1,2-a]pyrazine-2,3(1H)-dicarboxylate (930 mg) was dissolved in 5-10% hydrogen chloride-methanol solution (15 mL), and the solution was stirred at room temperature for 4 hr. The solvent was evaporated under reduced pressure to give the title compound (960 mg) as a yellow oil. Without further purification, the compound was used for the next reaction. LC-MS: 201 (MH⁺).

### (vii) Production of methyl(3S,7R,8aR)-2-{(2S)-2-[(tert-butoxycarbonyl)amino]-2-cyclohexylacetyl}-7-hydroxyoctahydropyrrolo[1,2-a]pyrazine-3-carboxylate

To a suspension of methyl(3S,7R,8aR)-7-hydroxyoctahydropyrrolo[1,2-a]pyrazine-3-carboxylate dihydrochloride (960 mg) in N,N-dimethylformamide (18 mL) were added N-ethyldiisopropylamine (2.70 mL), (2S)-[(tert-butoxycarbonyl)amino](cyclohexyl)ethanoic acid (1.03 g) and O-(7-azabenzotriazol-1-yl)-N,N,N′,N′-tetramethyluronium hexafluorophosphate (1.77 g), and the mixture was stirred at room temperature for 4 hr. The reaction mixture was diluted with ethyl acetate (100 mL) and water (25 mL), and the aqueous layer was extracted with ethyl acetate (50 mL). The combined organic layers were washed with saturated aqueous sodium hydrogen carbonate solution (25 mL×2) and saturated brine (25 mL), and dried over anhydrous magnesium sulfate, and the insoluble material was filtered off. The filtrate was concentrated under reduced pressure, and the residue was purified by silica gel column chromatography (ethyl acetate/hexane=5/95→100/0). The fraction containing the title compound was concentrated under reduced pressure, and the residue was dissolved in ethyl acetate (100 mL). The organic layer was washed with saturated aqueous sodium hydrogen carbonate solution (50 mL), and dried over anhydrous magnesium sulfate, and the insoluble material was filtered off. The filtrate was concentrated under reduced pressure to give the title compound (1.03 g) as a colorless amorphous powder. LC-MS: 440.3 (MH⁺).

¹H NMR (DMSO-d₆, 300 MHz): δ 0.82-1.29 (6H, m), 1.32-1.39 (9H, m), 1.53-1.88 (6H, m), 2.07-2.28 (3H, m), 2.75-2.85 (1H, m), 2.91-3.06 (1H, m), 3.36-3.50 (1H, m), 3.59-3.72 (3H, m), 4.11-4.41 (3H, m), 4.72-4.83 (1H, m), 5.03-5.09 (1H, m), 6.69-6.86 (1H, m), 7.22-7.37 (1H, m).

### (viii) Production of methyl(3S,7R,8aR)-2-[(2S)-2-amino-2-cyclohexylacetyl]-7-hydroxyoctahydropyrrolo[1,2-a]pyrazine-3-carboxylate

Methyl(3S,7R,8aR)-2-{(2S)-2-[(tert-butoxycarbonyl)amino]-2-cyclohexylacetyl}-7-hydroxyoctahydropyrrolo[1,2-a]pyrazine-3-carboxylate (1.03 g) was dissolved in 4N hydrogen chloride-ethyl acetate solution (10 mL), and the mixture was stirred at room temperature for 1 hr. The reaction mixture was diluted with water (30 mL), and the aqueous layer was separated, and washed with ethyl acetate (50 mL). The obtained aqueous layer was neutralized with 2N aqueous sodium hydroxide solution (4 mL), and extracted with ethyl acetate (100 mL×2). The combined organic layers were dried over anhydrous magnesium sulfate, and the insoluble material was filtered off. The filtrate was concentrated under reduced pressure to give the title compound (585 mg) as a colorless amorphous powder. Without further purification, the compound was used for the next reaction. LC-MS: 340.2 (MH⁺).

### (ix) Production of methyl(3S,7R,8aR)-2-[(2S)-2-{[N-(tert-butoxycarbonyl)-N-methyl-L-alanyl]amino}-2-cyclohexylacetyl]-7-hydroxyoctahydropyrrolo[1,2-a]pyrazine-3-carboxylate

To a solution of methyl(3S,7R,8aR)-2-[(2S)-2-amino-2-cyclohexylacetyl]-7-hydroxyoctahydropyrrolo[1,2-a]pyrazine-3-carboxylate (580 mg) in N,N-dimethylformamide (18 mL) were added N-(tert-butoxycarbonyl)-N-methyl-L-alanine (458 mg) and O-(7-azabenzotriazol-1-yl)-N,N,N′,N′-tetramethyluronium hexafluorophosphate (988 mg), and the mixture was stirred at room temperature for 18 hr. The reaction mixture was diluted with ethyl acetate (100 mL) and saturated aqueous sodium JO hydrogen carbonate solution (50 mL), and the aqueous layer was extracted with ethyl acetate (100 mL). The combined organic layers were washed with saturated brine (50 mL), and dried over anhydrous magnesium sulfate, and the insoluble material was filtered off. The filtrate was concentrated under reduced pressure, and the residue was purified by silica gel column chromatography (ethyl acetate/hexane=10/90→100/0) and basic silica gel column chromatography (ethyl acetate/hexane=20/80→100/0) to give the title compound (166 mg) as a colorless oil. LC-MS: 525.4 (MH⁺).

¹H NMR (DMSO-d₆, 300 MHz): δ 0.81-1.03 (2H, m), 1.05-1.27 (7H, m), 1.39 (9H, br s), 1.53-1.84 (7H, m), 2.05-2.26 (3H, m), 2.70-2.80 (4H, m), 2.91-3.04 (1H, m), 3.35-3.50 (1H, m), 3.61-3.69 (3H, m), 4.07-4.38 (2H, m), 4.44-4.59 (1H, m), 4.65-4.74 (1H, m), 4.77-4.84 (1H, m), 5.03-5.21 (1H, m), 7.34-7.92 (1H, m).

### (x) Production of tert-butyl[(1S)-2-({(1S)-1-cyclohexyl-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}amino)-1-methyl-2-oxoethyl]methylcarbamate

To a solution of methyl(3S,7R,8aR)-2-[(2S)-2-{[N-(tert-butoxycarbonyl)-N-methyl-L-alanyl]amino}-2-cyclohexylacetyl]-7-hydroxyoctahydropyrrolo[1,2-a]pyrazine-3-carboxylate (166 mg) in a mixed solvent (6.25 mL) of tetrahydrofuran/water (4/1) was added lithium hydroxide monohydrate (20 mg), and the mixture was stirred with heating at 50° C. for 2 hr. The reaction mixture was allowed to cool to room temperature, and neutralized with 1N hydrochloric acid (0.48 mL), and the solvent was evaporated under reduced pressure. The residue was dissolved in N,N-dimethylformamide (7 mL), (4R)-3,4-dihydro-2H-chromen-4-amine hydrochloride (88 mg), 1-hydroxybenzotriazole (42.7 mg), N-ethyldiisopropylamine (0.11 mL) and N-[3-(dimethylamino)propyl]-N′-ethylcarbodiimide-hydrochloride (400 mg) were added thereto, and the mixture was stirred at room temperature for 16 hr. To the reaction mixture were added 1-hydroxybenzotriazole (21.4 mg), N-ethyldiisopropylamine (0.11 mL) and N-[3-(dimethylamino)propyl]-N′-ethylcarbodiimide hydrochloride (200 mg), and the mixture was stirred again at room temperature for 24 hr. The reaction mixture was diluted with ethyl acetate (100 mL) and saturated aqueous sodium hydrogen carbonate solution (50 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0→ethyl acetate/methanol=80:20) to give the title compound (81 mg) as a yellow amorphous powder. LC-MS: 642.4 (MH⁺).

¹H NMR (CDCl₃, 300 MHz): δ 0.63-0.80 (1H, m), 0.80-1.02 (3H, m), 1.04-1.17 (2H, m), 1.17-1.30 (2H, m), 1.34-1.56 (12H, m), 1.57-1.86 (3H, m), 1.87-2.00 (1H, m), 2.08-2.45 (7H, m), 2.49-2.60 (1H, m), 2.65-2.79 (3H, m), 2.97-3.14 (2H, m), 3.68-3.91 (1H, m), 3.98-4.09 (1H, m), 4.15-4.39 (3H, m), 4.57 (1H, br s), 4.63-4.82 (1H, m), 5.03-5.11 (1H, m), 5.24 (1H, d, J=3.4 Hz), 6.34-7.90 (5H, m).

### (xi) Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxyoctahydropyrrolo[1,2-a]pyrazine-3-carboxamide dihydrochloride

tert-Butyl[(1S)-2-({(1S)-1-cyclohexyl-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}amino)-1-methyl-2-oxoethyl]methylcarbamate (81 mg) was dissolved in 10% hydrogen chloride-methanol solution (6.3 mL), and the solution was stirred at room temperature for 4.5 hr. The solvent was evaporated under reduced pressure, ethyl acetate (1 mL) and diethyl ether (3 mL) were added to the residue, and the precipitate was collected by filtration to give the title compound (53 mg) as a yellow amorphous powder. LC-MS: 542.4 (MH⁺).

¹H NMR (DMSO-d₆, 300 MHz): δ 0.93-1.26 (6H, m), 1.36 (3H, d, J=6.9 Hz), 1.55-2.08 (9H, m), 2.16-2.32 (1H, m), 2.42-2.47 (3H, m), 3.12-3.26 (1H, m), 3.41-3.54 (1H, m), 3.61-3.96 (4H, m), 4.14-4.24 (2H, m), 4.35-4.52 (2H, m), 4.63 (1H, dd, J=12.7, 5.5 Hz), 4.73-4.81 (1H, m), 4.94-5.04 (1H, m), 5.77 (1H, br s), 6.71-6.90 (2H, m), 7.01-7.23 (2H, m), 8.74-8.99 (3H, m), 9.32 (1H, br s), 12.14 (1H, br s).

### Example 41

### Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxy-7-methyloctahydropyrrolo[1,2-a]pyrazine-3-carboxamide dihydrochloride

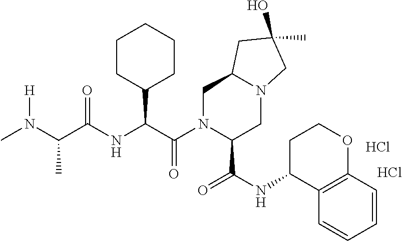

### (i) Production of 2-tert-butyl 3-methyl(3S,7R,8aR)-7-hydroxy-7-methylhexahydropyrrolo[1,2-a]pyrazine-2,3(1H)-dicarboxylate

A solution of 2-tert-butyl 3-methyl(3S,8aR)-7-oxohexahydropyrrolo[1,2-a]pyrazine-2,3(1H)-dicarboxylate (1.80 g) in tetrahydrofuran (24 mL) was cooled to −78° C., 1.0M methyllithium diethyl ether solution (9.0 mL) was added thereto, and the mixture was stirred at the same temperature for 1 hr. To the reaction mixture was added saturated aqueous ammonium chloride solution (50 mL), and the mixture was allowed to warm to room temperature. The aqueous layer was extracted with ethyl acetate (100 mL×2) and a mixed solvent (100 mL) of ethyl acetate/tetrahydrofuran (2/1). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (680 mg) as a yellow oil. LC-MS: 315.2 (MH⁺).

¹H NMR (DMSO-d₆, 300 MHz): δ 1.20 (3H, s), 1.32-1.52 (10H, m), 1.74-1.84 (1H, m), 1.86-1.98 (2H, m), 2.14-2.24 (1H, m), 2.59-2.85 (1H, m), 2.87 (1H, d, J=9.4 Hz), 3.25-3.31 (1H, m), 3.67-3.68 (3H, m), 3.78-3.89 (1H, m), 4.50-4.67 (2H, m).

### (ii) Production of methyl(3S,7R,8aR)-2-{(2S)-2-[(tert-butoxycarbonyl)amino]-2-cyclohexylacetyl}-7-hydroxy-7-methyloctahydropyrrolo[1,2-a]pyrazine-3-carboxylate

2-tert-Butyl 3-methyl(3S,7R,8aR)-7-hydroxy-7-methylhexahydropyrrolo[1,2-a]pyrazine-2,3(1H)-dicarboxylate (750 mg) was dissolved in 5-10% hydrogen chloride-methanol solution (15 mL), and the mixture was stirred at room temperature for 5 hr. To the reaction mixture was added 5-10% hydrogen chloride-methanol solution (15 mL), and the mixture was stirred again at room temperature for 2 hr. The solvent was evaporated under reduced pressure, and the resulting yellow amorphous powder was dissolved in N,N-dimethylformamide (15 mL). N-Ethyldiisopropylamine (2.20 mL), (2S)-[(tert-butoxycarbonyl)amino](cyclohexyl)ethanoic acid (850 mg) and O-(7-azabenzotriazol-1-yl)-N,N,N′,N′-tetramethyluronium hexafluorophosphate (1.45 g) were added thereto, and the mixture was stirred at room temperature for 14 hr. The reaction mixture was diluted with ethyl acetate (100 mL) and saturated aqueous sodium hydrogen carbonate solution (100 mL), and the aqueous layer was extracted with a mixed solvent (125 mL) of ethyl acetate/tetrahydrofuran (4/1). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (806 mg) as a yellow oil. LC-MS: 454.3. (MH⁺).

¹H NMR (DMSO-d₆, 300 MHz): δ 0.85-1.02 (2H, m), 1.07-1.25 (6H, m), 1.31-1.39 (9H, m), 1.42-1.53 (1H, m), 1.55-1.71 (5H, m), 1.74-1.84 (1H, m), 1.91-1.98 (1H, m), 2.08-2.19 (1H, m), 2.69 (3H, s), 2.85-3.05 (1H, m), 3.34-3.46 (1H, m), 3.60-3.78 (3H, m), 4.08-4.21 (1H, m), 4.26-4.40 (1H, m), 4.60-4.70 (1H, m), 5.01-5.19 (1H, m), 6.72-6.85 (1H, m).

### (iii) Production of methyl(3S,7R,8aR)-2-[(2S)-2-{[N-(tert-butoxycarbonyl)-N-methyl-L-alanyl]amino}-2-cyclohexylacetyl]-7-hydroxy-7-methyloctahydropyrrolo[1,2-a]pyrazine-3-carboxylate

Methyl(3S,7R,8aR)-2-{(2S)-2-[(tert-butoxycarbonyl)amino]-2-cyclohexylacetyl}-7-hydroxy-7-methyloctahydropyrrolo[1,2-a]pyrazine-3-carboxylate (806 mg) was dissolved in 4N hydrogen chloride-ethyl acetate solution (8 mL), and the mixture was stirred at room temperature for 30 min. The solvent was evaporated under reduced pressure, the residue was diluted with saturated aqueous sodium hydrogen carbonate solution (30 mL), and water was evaporated. The residue was diluted with ethyl acetate (50 mL), the insoluble material was filtered off, and the filtrate was concentrated under reduced pressure. The residue was dissolved in N,N-dimethylformamide (10 mL), N-(tert-butoxycarbonyl)-N-methyl-L-alanine (366 mg) and O-(7-azabenzotriazol-1-yl)-N,N,N′,N′-tetramethyluronium hexafluorophosphate (821 mg) were added thereto, and the mixture was stirred at room temperature for 3 hr. The reaction mixture was diluted with ethyl acetate (100 mL) and saturated aqueous sodium hydrogen carbonate solution (50 mL), and the aqueous layer was extracted with a mixed solvent (120 mL) of ethyl acetate/tetrahydrofuran (5/1). The combined organic layers were dried over anhydrous magnesium sulfate, and the insoluble material was filtered off. The filtrate was concentrated under reduced pressure, and the residue was purified by silica gel column chromatography (ethyl acetate/hexane=20/80→100/0→ethyl acetate/methanol=80/20) and basic silica gel column chromatography (ethyl acetate/hexane=10/90→100/0) to give the title compound (80 mg) as a yellow oil. LC-MS: 539.3. (MH⁺).

¹H NMR (CDCl₃, 300 MHz): δ 0.77-1.38 (14H, m), 1.38-1.53 (9H, m), 1.54-1.91 (6H, m), 2.04 (3H, s), 2.25-2.38 (1H, m), 2.39-2.52 (1H, m), 2.86-3.00 (2H, m), 3.10-3.28 (1H, m), 3.50-3.65 (1H, m), 3.74 (3H, s), 3.95-4.07 (1H, m), 4.52-4.79 (1H, m), 4.83-4.98 (1H, m), 5.27-5.45 (1H, m), 6.38-7.00 (1H, m),

### (iv) Production of tert-butyl[(1S)-2-({(1S)-1-cyclohexyl-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}amino)-1-methyl-2-oxoethyl]methylcarbamate

To a solution of methyl(3S,7R,8aR)-2-[(2S)-2-{[N-(tert-butoxycarbonyl)-N-methyl-L-alanyl]amino}-2-cyclohexylacetyl]-7-hydroxy-7-methyloctahydropyrrolo[1,2-a]pyrazine-3-carboxylate (77 mg) in a mixed solvent (3.13 mL) of tetrahydrofuran/water (4/1) was added lithium hydroxide monohydrate (9.0 mg), and the mixture was stirred with heating at 60° C. for 3 hr. The reaction mixture was allowed to cool to room temperature, and neutralized with 1N hydrochloric acid (0.2 mL), and the solvent was evaporated under reduced pressure. The residue was dissolved in N,N-dimethylformamide (1.5 mL), (4R)-3,4-dihydro-2H-chromen-4-amine hydrochloride (40 mg), N-ethyldiisopropylamine (0.05 mL) and O-(7-azabenzotriazol-1-yl)-N,N,N′,N′-tetramethyluronium hexafluorophosphate (272 mg) were added thereto, and the mixture was stirred at room temperature for 16 hr. The reaction mixture was diluted with ethyl acetate (100 mL) and saturated aqueous sodium hydrogen carbonate solution (100 mL), and the aqueous layer was extracted with a mixed solvent (100 mL) of ethyl acetate/tetrahydrofuran (4/1). The combined organic layers were dried over anhydrous magnesium sulfate, and the insoluble material was filtered off. The filtrate was concentrated under reduced pressure, and the residue was purified by silica gel column chromatography (ethyl acetate/hexane=20/80→100/0→ethyl acetate/methanol=80/20) and basic silica gel column chromatography (ethyl acetate/hexane=10/90→80/20) to give the title compound (41.4 mg) as a colorless amorphous powder. LC-MS: 656.4 (MH⁺).

¹H NMR (CDCl₃, 300 MHz): δ 0.64-1.39 (11H, m), 1.40-1.51 (9H, m), 1.60-1.85 (5H, m), 1.94-2.10 (3H, m), 2.13-2.30 (3H, m), 2.41-2.65 (2H, m), 2.64-2.84 (3H, m), 2.96-3.35 (2H, m), 3.61-4.41 (5H, m), 4.56-4.84 (2H, m), 5.06-5.32 (2H, m), 6.28-7.95 (6H, m).

### (v) Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxy-7-methyloctahydropyrrolo[1,2-a]pyrazine-3-carboxamide dihydrochloride

tert-Butyl[(1S)-2-({(1S)-1-cyclohexyl-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}amino)-1-methyl-2-oxoethyl]methylcarbamate (41.4 mg) was dissolved in 4N hydrogen chloride-ethyl acetate solution (2 mL), and the solution was stirred at room temperature for 30 min. The solvent was evaporated under reduced pressure, and the resulting solid was washed with ethyl acetate (0.5 mL) and diethyl ether (2.0 mL), and filtered to give the title compound (30.5 mg) as a colorless amorphous powder. LC-MS: 556.4 (MH⁺).

¹H NMR (DMSO-d₆, 300 MHz): δ 0.96-1.25 (7H, m), 1.29-1.40 (6H, m), 1.60-2.19 (11H, m), 3.16-3.28 (2H, m), 3.70-3.95 (5H, m), 4.15-4.25 (2H, m), 4.35-4.49 (1H, m), 4.58-4.68 (1H, m), 4.73-4.83 (1H, m), 4.93-5.05 (1H, m), 5.58 (1H, br s), 6.71-6.91 (2H, m), 7.06-7.22 (2H, m), 8.75-8.94 (3H, m), 9.11-9.24 (1H, m), 11.87-12.06 (1H, m).

### Example 42

### Production of (3S,8aR)-2-{(2S)-2-cyclohexyl-2-[(N-methyl-L-alanyl)amino]acetyl}-N-[(4R)-3,4-dihydro-2H-chromen-4-yl]-7-oxooctahydropyrrolo[1,2-a]pyrazine-3-carboxamide dihydrochloride

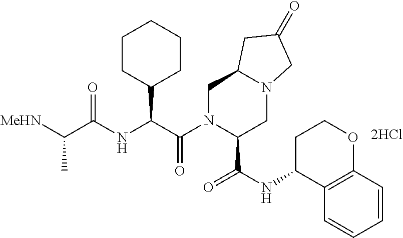

### (i) Production of methyl(3S,8aR)-2-{(2S)-2-[(tert-butoxycarbonyl)amino]-2-cyclohexylacetyl}-7,7-dimethoxyoctahydropyrrolo[1,2-a]pyrazine-3-carboxylate

2-tert-Butyl 3-methyl(3S,8aR)-7-oxohexahydropyrrolo[1,2-a]pyrazine-2,3(1H)-dicarboxylate (860 mg) was dissolved in 5-10% hydrogen chloride-methanol solution (15 mL), and the solution was stirred at room temperature for 5 hr. The solvent was evaporated under reduced pressure, and the resulting brown amorphous powder was dissolved in N,N-dimethylformamide (18 mL). N-ethyldiisopropylamine (2.70 mL), (2S)-[(tert-butoxycarbonyl)amino](cyclohexyl)ethanoic acid (1.03 g) and O-(7-azabenzotriazol-1-yl)-N,N,N′,N′-tetramethyluronium hexafluorophosphate (1.77 g) were added thereto, and the mixture was stirred at room temperature for 14 hr. The reaction mixture was diluted with ethyl acetate (100 mL) and saturated aqueous sodium hydrogen carbonate solution (50 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=0/100→80/20) to give the title compound (900 mg) as an amorphous powder. LC-MS: 484.3 (MH⁺).

¹H NMR (DMSO-d₆, 300 MHz): δ 0.79-1.27 (6H, m), 1.30-1.42 (9H, m), 1.45-1.78 (6H, m), 1.85-2.42 (3H, m), 2.43-2.63 (1H, m), 2.65-2.76 (2H, m), 2.81-3.16 (5H, m), 3.23-3.46 (1H, m), 3.48-3.74 (4H, m), 4.10-4.54 (2H, m), 5.03-5.38 (1H, m), 6.69-6.95 (1H, m).

### (ii) Production of tert-butyl{(1S)-1-cyclohexyl-2-[(3S,8aR)-3-[(4R)-3,4-dihydro-2H-chromen-4-ylcarbamoyl]-7,7-dimethoxyhexahydropyrrolo[1,2-a]pyrazin-2(1H)-yl]-2-oxoethyl}carbamate

To a solution of methyl(3S,8aR)-2-{(2S)-2-[(tert-butoxycarbonyl)amino]-2-cyclohexylacetyl}-7,7-dimethoxyoctahydropyrrolo[1,2-a]pyrazine-3-carboxylate (213 mg) in a mixed solvent (10 mL) of tetrahydrofuran/water (4/1) was added lithium hydroxide monohydrate (27.8 mg), and the mixture was stirred with heating at 60° C. for 1 hr. The reaction mixture was allowed to cool to room temperature, and neutralized with 1N hydrochloric acid (0.8 mL), and the solvent was evaporated under reduced pressure. The residue was dissolved in N,N-dimethylformamide (5 mL), (4R)-3,4-dihydro-2H-chromen-4-amine hydrochloride (123 mg), N-ethyldiisopropylamine (0.155 mL) and O-(7-azabenzotriazol-1-yl)-N,N,N′,N′-tetramethyluronium hexafluorophosphate (838 mg) were added thereto, and the mixture was stirred at room temperature for 16 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (231 mg) as a yellow oil. LC-MS: 601.4 (MH⁺).

¹H NMR (CDCl₃, 300 MHz): δ 0.60-1.34 (9H, m), 1.36-1.88 (11H, m), 1.96-2.11 (2H, m), 2.16-2.39 (4H, m), 2.49-2.63 (1H, m), 2.68-2.88 (1H, m), 3.01-3.12 (1H, m), 3.16-3.22 (5H, m), 3.23-3.37 (1H, m), 3.59-3.96 (1H, m), 3.97-4.32 (3H, m), 4.44-4.54 (1H, m), 4.66-4.76 (0.5H, m), 5.06-5.21 (1.5H, m), 5.25-5.37 (1H, m), 6.45-8.22 (5H, m).

### (iii) Production of tert-butyl[(1S)-2-({(1S)-1-cyclohexyl-2-[(3S,8aR)-3-[(4R)-3,4-dihydro-2H-chromen-4-ylcarbamoyl]-7-oxohexahydropyrrolo[1,2-a]pyrazin-2(1H)-yl]-2-oxoethyl}amino)-1-methyl-2-oxoethyl]methylcarbamate

tert-Butyl{(1S)-1-cyclohexyl-2-[(3S,8aR)-3-[(4R)-3,4-dihydro-2H-chromen-4-ylcarbamoyl]-7,7-dimethoxyhexahydropyrrolo[1,2-a]pyrazin-2(1H)-yl]-2-oxoethyl}carbamate (231 mg) was dissolved in 5-10% hydrogen chloride-methanol solution (20 mL), and the mixture was stirred at room temperature for 4 hr. The reaction mixture was warmed to 50° C., and stirred for 30 min. The reaction mixture was allowed to cool to room temperature, and the solvent was evaporated under reduced pressure. The residue was dissolved in N,N-dimethylformamide (5 mL), N-ethyldiisopropylamine (0.27 mL), N-(tert-butoxycarbonyl)-N-methyl-L-alanine (102 mg) and O-(7-azabenzotriazol-1-yl)-N,N,N′,N′-tetramethyluronium hexafluorophosphate (220 mg) were added thereto, and the mixture was stirred at room temperature for 60 hr. The reaction mixture was diluted with ethyl acetate (75 mL) and saturated aqueous sodium hydrogen carbonate solution (75 mL), and the aqueous layer was extracted with ethyl acetate (75 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0) to give the title compound (140 mg) as a yellow oil. LC-MS: 640.4 (MH⁺).

¹H NMR (CDCl₃, 300 MHz): δ 0.63-1.03 (3H, m), 1.05-1.30 (4H, m), 1.44-1.51 (9H, m), 1.66-1.80 (2H, m), 1.81-1.90 (3H, m), 1.98-2.29 (3H, m), 2.32-2.81 (6H, m), 3.13-3.64 (1H, m), 3.69-4.71 (9H, m), 4.74-4.97 (1H, m), 5.09-5.24 (1H, m), 5.31-6.18 (1H, m), 6.68-6.96 (3H, m), 7.09-7.22 (2H, m), 7.80-7.92 (1H, m).

### (iv) Production of (3S,8aR)-2-{(2S)-2-cyclohexyl-2-[(N-methyl-L-alanyl)amino]acetyl}-N-[(4R)-3,4-dihydro-2H-chromen-4-yl]-7-oxooctahydropyrrolo[1,2-a]pyrazine-3-carboxamide

To a solution of tert-butyl[(1S)-2-({(1S)-1-cyclohexyl-2-[(3S,8aR)-3-[(4R)-3,4-dihydro-2H-chromen-4-ylcarbamoyl]-7-oxohexahydropyrrolo[1,2-a]pyrazin-2(1H)-yl]-2-oxoethyl}amino)-1-methyl-2-oxoethyl]methylcarbamate (140 mg) in tetrahydrofuran (1 mL) was added 6M hydrochloric acid (1 mL), and the mixture was stirred with heating at 50° C. for 2 hr. The reaction mixture was allowed to cool to room temperature, and diluted with ethyl acetate (50 mL) and saturated aqueous sodium hydrogen carbonate solution (30 mL), and the aqueous layer was extracted with ethyl acetate (50 mL). The combined organic layers were dried over anhydrous magnesium sulfate, and the insoluble material was filtered off. The filtrate was concentrated under reduced pressure to give the title compound (90 mg) as a yellow amorphous powder. LC-MS: 540.3. (MH⁺).

¹H NMR (CDCl₃, 300 MHz): δ 0.70-1.14 (4H, m), 1.16-1.34 (4H, m), 1.40-1.89 (6H, m), 1.92-2.29 (5H, m), 2.30-2.76 (6H, m), 2.87-3.10 (1H, m), 3.15-3.93 (2H, m), 3.98-4.42 (4H, m), 4.64-5.47 (3H, m), 6.15-8.04 (6H, m).

### (v) Production of (3S,8aR)-2-{(2S)-2-cyclohexyl-2-[(N-methyl-L-alanyl)amino]acetyl}-N-[(4R)-3,4-dihydro-2H-chromen-4-yl]-7-oxooctahydropyrrolo[1,2-a]pyrazine-3-carboxamide dihydrochloride

(3S,8aR)-2-{(2S)-2-Cyclohexyl-2-[(N-methyl-L-alanyl)amino]acetyl}-N-[(4R)-3,4-dihydro-2H-chromen-4-yl]-7-oxooctahydropyrrolo[1,2-a]pyrazine-3-carboxamide (90 mg) was dissolved in a mixed solvent of ethyl acetate (2 mL) and diethyl ether (2 mL), 4M hydrogen chloride-ethyl acetate solution (0.25 mL) was added thereto, and the solution was stirred at room temperature for 15 min. The solvent was evaporated under reduced pressure, and the resulting solid was washed with diethyl ether (2 mL) and ethyl acetate (1 mL), and collected by filtration to give the title compound (70 mg) as a pale-yellow solid. LC-MS: 540.3 (MH⁺).

¹H NMR (DMSO-d₆, 300 MHz): δ 0.95-1.26 (5H, m), 1.29-1.40 (3H, m), 1.52-1.84 (6H, m), 1.87-2.09 (2H, m), 2.44-2.49 (3H, m), 2.68-3.23 (1H, m), 3.48-3.98 (10H, m), 4.11-4.45 (3H, m), 4.58-5.17 (3H, m), 6.72-6.94 (2H, m), 7.07-7.38 (2H, m), 8.44-8.96 (2H, m), 9.23-9.43 (1H, m).

### Example 43

### Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-hydroxy-7-methyloctahydropyrrolo[1,2-a]pyrazine-3-carboxamide dihydrochloride

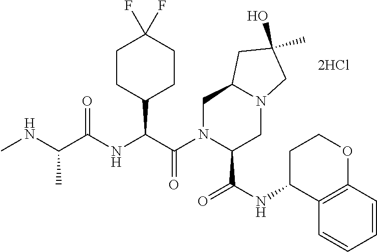

### (i) Production of tert-butyl(3S,7R,8aR)-3-[(4R)-3,4-dihydro-2H-chromen-4-ylcarbamoyl]-7-hydroxy-7-methylhexahydropyrrolo[1,2-a]pyrazine-2(1H)-carboxylate

To a solution of 2-tert-butyl 3-methyl(3S,7R,8aR)-7-hydroxy-7-methylhexahydropyrrolo[1,2-a]pyrazine-2,3(1H)-dicarboxylate (570 mg) in a mixed solvent of tetrahydrofuran/water (4/1) was added lithium hydroxide monohydrate (115 mg), and the mixture was stirred with heating at 60° C. for 3 hr. The reaction mixture was allowed to cool to room temperature, and neutralized with 1M hydrochloric acid (2 mL), and the solvent was evaporated under reduced pressure. The resulting residue was dissolved in N,N-dimethylformamide (18 mL), (4R)-3,4-dihydro-2H-chromen-4-amine hydrochloride (507 mg), N-ethyldiisopropylamine (6.50 mL) and O-(7-azabenzotriazol-1-yl)-N,N,N′,N′-tetramethyluronium hexafluorophosphate (3.46 g) were added thereto, and the mixture was stirred at room temperature for 14 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=5/95→100/0→ethyl acetate/methanol=80:20) to give the title compound (231 mg) as a yellow oil. LC-MS: 432.2 (MH⁺).

¹H NMR (DMSO-d₆, 300 MHz): δ 1.18-1.23 (3H, m), 1.32-1.56 (10H, m), 1.74-2.24 (6H, m), 2.64-3.12 (2H, m), 3.23-3.39 (1H, m), 3.74-3.91 (1H, m), 4.14-4.28 (2H, m), 4.32-4.52 (1H, m), 4.57-4.69 (1H, m), 4.88-5.16 (1H, m), 6.66-6.93 (2H, m), 7.07-7.20 (2H, m), 8.17-8.44 (1H, m).

### (ii) Production of (3S,7R,8aR)—N-[(4R)-3,4-dihydro-2H-chromen-4-yl]-7-hydroxy-7-methyloctahydropyrrolo[1,2-a]pyrazine-3-carboxamide dihydrochloride

tert-Butyl(3S,7R,8aR)-3-[(4R)-3,4-dihydro-2H-chromen-4-ylcarbamoyl]-7-hydroxy-7-methylhexahydropyrrolo[1,2-a]pyrazine-2(1H)-carboxylate (300 mg) was dissolved in 4M hydrogen chloride-ethyl acetate solution (10 mL), and the solution was stirred at room temperature for 1 hr. The solvent was evaporated under reduced pressure, and the resulting solid was washed with diethyl ether (10 mL), and collected by filtration to give the title compound (246 mg) as a white solid. LC-MS: 332.2 (MH⁺).

¹H NMR (DMSO-d₆, 300 MHz): δ 1.36 (3H, s), 1.82-2.01 (2H, m), 2.03-2.19 (2H, m), 2.23-2.45 (1H, m), 3.10-3.28 (1H, m), 3.30-3.49 (2H, m), 3.51-3.81 (4H, m), 3.87-4.09 (2H, m). 4.12-4.40 (3H, m), 4.94-5.14 (1H, m), 6.71-6.97 (2H, m), 7.09-7.35 (2H, m), 9.43 (1H, br s), 12.59 (1H, br s).

### (iii) Production of benzyl{(1S)-1-(4,4-difluorocyclohexyl)-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}carbamate

To a solution of (3S,7R,8aR)—N-[(4R)-3,4-dihydro-2H-chromen-4-yl]-7-hydroxy-7-methyloctahydropyrrolo[1,2-a]pyrazine-3-carboxamide dihydrochloride (245 mg) in N,N-dimethylformamide (5 mL) were added N-ethyldiisopropylamine (0.350 mL), (2S)-{[(benzyloxy)carbonyl]amino}(4,4-difluorocyclohexyl)ethanoic acid (250 mg) and O-(7-azabenzotriazol-1-yl)-N,N,N′,N′-tetramethyluronium hexafluorophosphate (381 mg), and the mixture was stirred at room temperature for 16 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (244 mg) as a pale-yellow amorphous powder. LC-MS: 641.3 (MH⁺).

¹H NMR (CDCl₃, 300 MHz): δ 1.20-1.28 (10H, m), 1.30-1.41 (2H, m), 1.60-2.06 (8H, m), 3.14 (2H, dd, J=7.4, 4.2 Hz), 3.22-3.48 (1H, m), 3.52-3.91 (2H, m), 4.14-4.27 (2H, m), 4.33-5.16 (4H, m), 6.72-6.92 (2H, m), 7.08-7.22 (2H, m), 7.26-7.43 (5H, m), 7.58-10.61 (2H, m).

### (iv) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}amino)-1-methyl-2-oxoethyl]methylcarbamate

To a solution of benzyl{(1S)-1-(4,4-difluorocyclohexyl)-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}carbamate (220 mg) in 5-10% hydrogen chloride-methanol (10 mL) was added 20% palladium hydroxide-carbon (44.0 mg, 50%, wet), and the mixture was stirred at room temperature for 3 hr under a hydrogen atmosphere. The insoluble material was filtered off through a pad filled with celite, and the filtrate was concentrated. The obtained residue was dissolved in N,N-dimethylformamide (5 mL), N-ethyldiisopropylamine (0.27 mL), N-(tert-butoxycarbonyl)-N-methyl-L-alanine (102 mg) and O-(7-azabenzotriazol-1-yl)-N,N,N′,N′-tetramethyluronium hexafluorophosphate (220 mg) were added thereto, and the mixture was stirred at room temperature for 2 hr. The reaction mixture was diluted with ethyl acetate (40 mL) and saturated aqueous sodium hydrogen carbonate solution (40 mL), and the aqueous layer was extracted with ethyl acetate (40 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) and basic silica gel column chromatography (ethyl acetate/hexane=10/90→100/0) to give the title compound (95 mg) as a colorless amorphous powder. LC-MS: 692.4 (MH⁺).

¹H NMR (CDCl₃, 300 MHz): δ 1.09-1.40 (9H, m), 1.42-1.52 (10H, m), 1.52-1.81 (5H, m), 1.88-2.38 (8H, m), 2.52-2.84 (4H, m), 2.91-3.62 (2H, m), 3.79-4.32 (3H, n), 4.41-4.75 (2H, m), 4.82-5.30 (2H, m), 6.30-7.67 (6H, m).

### (v) Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-hydroxy-7-methyloctahydropyrrolo[1,2-a]pyrazine-3-carboxamide dihydrochloride

tert-Butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-hydroxy-7-methylhexahydropyrrolo[1,2-a]pyrazin-2(1H)-yl]-2-oxoethyl}amino)-1-methyl-2-oxoethyl]methylcarbamate (95 mg) was dissolved in 4M hydrogen chloride-ethyl acetate solution (4 mL), and the solution was stirred at room temperature for 30 min. The solvent was evaporated under reduced pressure, and the resulting solid was washed with ethyl acetate (1 mL) and diethyl ether (2 mL), and collected by filtration to give the title compound (79 mg) as a white solid. LC-MS: 592.4 (MH⁺).

¹H NMR (DMSO-d₆, 300 MHz): δ 1.25-1.49 (8H, m), 1.60-1.97 (9H, m), 2.01-2.21 (3H, m), 2.40-2.48 (3H, m), 3.16-3.28 (2H, m), 3.63-3.99 (4H, m), 4.15-4.27 (2H, m), 4.39-4.73 (2H, m), 4.80-5.05 (2H, m), 5.61 (1H, br s), 6.58-7.02 (2H, m), 7.09-7.24 (2H, m) 8.57-9.01 (3H, m), 9.34 (1H, br s), 12.16-12.82 (1H, m).

### Example 44

### Production of (3S,8aR)—N-[(4R)-3,4-dihydro-2H-chromen-4-yl]-7,7-difluoro-2-{(2S)-2-[(N-methyl-L-alanyl)amino]-2-phenylacetyl}octahydropyrrolo[1,2-a]pyrazine-3-carboxamide dihydrochloride

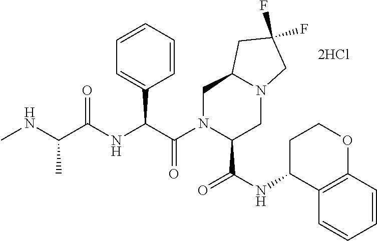

### (i) Production of tert-butyl{(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-phenylethyl}carbamate

A solution of (3S,8aR)—N-[(4R)-3,4-dihydro-2H-chromen-4-yl]-7,7-difluorooctahydropyrrolo[1,2-a]pyrazine-3-carboxamide dihydrochloride (250 mg) in N,N-dimethylformamide (5 mL) was cooled to 0° C., N-ethyldiisopropylamine (0.64 mL), (2S)-[(tert-butoxycarbonyl)amino](phenyl)ethanoic acid (230 mg) and O-(7-azabenzotriazol-1-yl)-N,N,N′,N′-tetramethyluronium hexafluorophosphate (463 mg) were added thereto, and the mixture was stirred at the same temperature for 30 min. The reaction mixture was allowed to warm to room temperature and stirred for 5 hr. The reaction mixture was diluted with ethyl acetate (80 mL) and saturated aqueous sodium hydrogen carbonate solution (50 mL), and the aqueous layer was extracted with ethyl acetate (80 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→80/20) to give the title compound (303 mg) as a pale-yellow amorphous powder. LC-MS: 571.3 (MH⁺).

¹H NMR (CDCl₃, 300 MHz): δ 1.25-1.47 (9H, m), 1.65-2.68 (8H, m), 3.37-3.53 (1H, m), 3.63-4.01 (2H, m), 4.06-4.39 (2H, m), 4.63-5.04 (1H, m), 5.19-5.32 (1H, m), 5.35-5.52 (2H, m), 5.58-6.06 (1H, m), 6.66-7.25 (6H, m), 7.30-7.60 (3H, m).

### (ii) Production of (3S,8aR)-2-[(2S)-2-amino-2-phenylacetyl]-N-[(4R)-3,4-dihydro-2H-chromen-4-yl]-7,7-difluorooctahydropyrrolo[1,2-a]pyrazine-3-carboxamide dihydrochloride

tert-Butyl{(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-phenylethyl}carbamate (303 mg) was dissolved in 4N hydrogen chloride-cyclopentyl methyl ether solution (5.3 mL), and the solution was stirred at room temperature for 30 min. To the reaction mixture was added 4M hydrogen chloride-cyclopentyl methyl ether solution (5.3 mL), and the mixture was stirred at room temperature for 1 hr. The reaction mixture was diluted with diethyl ether (20 mL), and the resulting solid was collected by filtration, and washed with diethyl ether (10 mL) to give the title compound (245 mg) as a gray solid. LC-MS: 471.3 (MH⁺).

¹H NMR (DMSO-d₆, 300 MHz): δ 1.38-2.14 (4H, m), 2.22-2.47 (2H, m), 2.59-3.09 (2H, m), 3.20-3.65 (2H, m), 3.83-4.06 (1H, m), 4.11-4.28 (2H, m), 4.34-4.58 (3H, m), 4.88-5.19 (2H, m), 5.29-5.85 (1H, m), 6.69-6.82 (1H, m), 6.83-6.95 (1H, m), 7.11-7.30 (2H, m), 7.41-7.63 (4H, m), 8.51-8.82 (3H, m).

### (iii) Production of tert-butyl[(1S)-2-({(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-phenylethyl}amino)-1-methyl-2-oxoethyl]methylcarbamate

A solution of N-(tert-butoxycarbonyl)-N-methyl-L-alanine (138 mg) in N,N-dimethylformamide (3 mL) was cooled to 0° C., 1-hydroxybenzotriazole (68 mg) and N-[3-(dimethylamino)propyl]-N′-ethylcarbodiimide hydrochloride (173 mg) were added thereto, and the reaction mixture was allowed to warm to room temperature and stirred at the same temperature for 30 min. A solution of (3S,8aR)-2-[(2S)-2-amino-2-phenylacetyl]-N-[(4R)-3,4-dihydro-2H-chromen-4-yl]-7,7-difluorooctahydropyrrolo[1,2-a]pyrazine-3-carboxamide dihydrochloride (245 mg) and N-ethyldiisopropylamine (0.47 mL) in N,N-dimethylformamide (2 mL) was added thereto, and the mixture was stirred at room temperature for 2 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→400/0) to give the title compound (303 mg) as a colorless amorphous powder. LC-MS 656.4 (MH⁺).

¹H NMR (DMSO-d₆, 300 MHz): δ 1.11-1.47 (13H, m), 1.69-1.92 (2H, m), 2.05-2.17 (1H, m), 2.26-2.46 (3H, m), 2.58-2.80 (4H, m), 2.89-3.17 (1H, m), 3.34-3.60 (1H, m), 3.87-4.73 (4H, m), 4.98-5.21 (2H, m), 5.61-6.05 (1H, m), 6.71-6.93 (2H, m), 7.09-7.46 (7H, m), 8.03-8.55 (2H, m).

### (iv) Production of (3S,8aR)—N-[(4R)-3,4-dihydro-2H-chromen-4-yl]-7,7-difluoro-2-{(2S)-2-[(N-methyl-L-alanyl)amino]-2-phenylacetyl}octahydropyrrolo[1,2-a]pyrazine-3-carboxamide dihydrochloride

tert-Butyl[(1S)-2-({(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-phenylethyl}amino)-1-methyl-2-oxoethyl]methylcarbamate (204 mg) was dissolved in 4M hydrogen chloride-ethyl acetate solution (2 mL), and the solution was stirred at room temperature for 30 min. The reaction mixture was diluted with diethyl ether (20 mL), and the resulting solid was collected by filtration, and washed with diethyl ether (10 mL) to give the title compound (147 mg) as a colorless solid. LC-MS: 556.3

¹H NMR (DMSO-d₆, 300 MHz): δ 1.04-1.48 (4H, m), 1.76-2.12 (3H, m), 2.30-2.49 (4H, m), 2.76-3.64 (3H, m), 3.81-4.62 (4H, m), 4.88-6.22 (5H, m), 6.73-6.97 (2H, 10, 7.11-7.63 (8H, m), 8.33-9.66 (4H, m).

### Example 45

### Production of (3S,7R,8aR)—N-[(4R)-3,4-dihydro-2H-chromen-4-yl]-7-ethoxy-2-{(2S)-2-(4-fluorophenyl)-2-[(N-methyl-L-alanyl)amino]acetyl}octahydropyrrolo[1,2-a]pyrazine-3-carboxamide

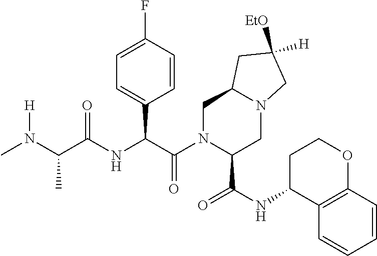

### (i) Production of tert-butyl[(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-1-(4-fluorophenyl)-2-oxoethyl]carbamate

To a solution of (3S,7R,8aR)—N-[(4R)-3,4-dihydro-2H-chromen-4-yl]-7-ethoxyoctahydropyrrolo[1,2-a]pyrazine-3-carboxamide dihydrochloride (417 mg) in N,N-dimethylformamide (8.8 mL) were added (2S)-[(tert-butoxycarbonyl)amino](4-fluorophenyl)ethanoic acid (370 mg) and O-(7-azabenzotriazol-1-yl)-N,N,N′,N′-tetramethyluronium hexafluorophosphate (570 mg) under ice-cooling, and the reaction mixture was allowed to warm to room temperature and stirred for 18 hr. To the reaction mixture were added O-(7-azabenzotriazol-1-yl)-N,N,N′,N′-tetramethyluronium hexafluorophosphate (570 mg) and N-ethyldiisopropylamine (0.7 mL), and the mixture was stirred again at room temperature for 3 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→80/20) to give the title compound (350 mg) as a yellow amorphous powder. LC-MS: 597.4 (MH⁺).

¹H NMR (DMSO-d₆, 300 MHz): δ 1.08 (3H, t, J=7.0 Hz), 1.19-1.25 (2H, m), 1.26-1.70 (10H, m), 1.80-2.04 (3H, m), 2.08-2.21 (2H, m), 2.85-3.00 (2H, m), 3.28-3.38 (2H, m), 3.41-3.64 (1H, m), 3.85-4.51 (4H, m), 4.89-4.99 (1H, m), 5.00-5.18 (1H, m), 5.45-5.76 (1H, m), 6.71-6.99 (2H, m), 7.09-7.63 (6H, m), 7.94-8.54 (1H, m).

### (ii) Production of (3S,7R,8aR)-2-[(2S)-2-amino-2-(4-fluorophenyl)acetyl]-N-[(4R)-3,4-dihydro-2H-chromen-4-yl]-7-ethoxyoctahydropyrrolo[1,2-a]pyrazine-3-carboxamide dihydrochloride

tert-Butyl[(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-1-(4-fluorophenyl)-2-oxoethyl]carbamate (350 mg) was dissolved in 4M hydrogen chloride-ethyl acetate solution (6.0 mL), and the solution was stirred at room temperature for 1 hr. The reaction mixture was diluted with diethyl ether (20 mL), and the resulting solid was collected by filtration, and washed with diethyl ether (10 mL) to give the title compound (260 mg) as a yellow solid. LC-MS: 497.3 (MH⁺).

¹H NMR (DMSO-d₆, 300 MHz): δ 0.98-1.14 (3H, m), 3.04-3.25 (1H, m), 3.24-3.43 (9H, m), 3.68-3.95 (1H, m), 3.96-4.52 (5H, m), 4.56-4.87 (1H, m), 4.87-5.20 (1H, m), 5.53-6.04 (1H, m), 6.80 (1H, d, J=8.1 Hz), 6.92 (1H, t, J=7.5 Hz), 7.11-7.45 (4H, m), 7.66 (2H, dd J=5.5, 8.5 Hz), 8.74 (3H, br s), 8.97-9.69 (1H, m), 9.88-12.90 (1H, m).

### (iii) Production of tert-butyl[(1S)-2-{[(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-1-(4-fluorophenyl)-2-oxoethyl]amino}-1-methyl-2-oxoethyl]methylcarbamate

A solution of N-(tert-butoxycarbonyl)-N-methyl-L-alanine (155 mg) in N,N-dimethylformamide (3 mL) was cooled to 0° C., 1-hydroxybenzotriazole (68 mg) and N-[3-(dimethylamino)propyl]-N′-ethylcarbodiimide hydrochloride (260 mg) were added thereto, and the reaction mixture was allowed to warm to room temperature and stirred at the same temperature for 30 min. A solution of (3S,7R,8aR)-2-[(2S)-2-amino-2-(4-fluorophenyl)acetyl]-N-[(4R)-3,4-dihydro-2H-chromen-4-yl]-7-ethoxyoctahydropyrrolo[1,2-a]pyrazine-3-carboxamide dihydrochloride (260 mg) in N,N-dimethylformamide (2 mL) and N-ethyldiisopropylamine (0.48 mL) were added thereto, and the mixture was stirred at room temperature for 3 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (175 mg) as a colorless amorphous powder. LC-MS 682.4. (MH⁺).

¹H NMR (DMSO-d₆, 300 MHz): δ 0.84-0.95 (1H, m), 1.01-1.16 (3H, m), 1.23-1.32 (5H, m), 1.32-1.59 (10H, m), 1.71-2.32 (5H, m), 2.44-2.73 (3H, m), 2.85-3.15 (1H, m), 3.20-3.42 (2H, m), 3.60-3.75 (1H, m), 3.80-4.30 (4H, m), 4.57-4.87 (1H, m), 4.89-5.92 (2H, m), 5.92-6.39 (1H, m), 6.71-7.45 (8H, m), 7.46-8.42 (1H, m).

### (iv) Production of (3S,7R,8aR)—N-[(4R)-3,4-dihydro-2H-chromen-4-yl]-7-ethoxy-2-{(2S)-2-(4-fluorophenyl)-2-[(N-methyl-L-alanyl)amino]acetyl}octahydropyrrolo[1,2-a]pyrazine-3-carboxamide

tert-Butyl[(1S)-2-{[(1S)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-1-(4-fluorophenyl)-2-oxoethyl]amino}-1-methyl-2-oxoethyl]methylcarbamate (175 mg) was dissolved in 4M hydrogen chloride-ethyl acetate solution (5 mL), and the solution was stirred at room temperature for 1 hr. The reaction mixture was diluted with ethyl acetate (100 mL) and saturated aqueous sodium hydrogen carbonate solution (100 mL), and the aqueous layer was extracted with ethyl acetate (100 mL). The combined organic layers were dried over anhydrous magnesium sulfate, and the insoluble material was filtered off. The filtrate was concentrated under reduced pressure, and the obtained residue was purified by preparative HPLC [Apparatus: Gilson, Inc. high throughput purification system, Column: CombiPrepPro C18RS (50×20 mm S-5 μm), Solvent: SOLUTION A; 0.1% trifluoroacetic acid-containing water, SOLUTION B; 0.1% trifluoroacetic acid containing acetonitrile, Gradient cycle: 0.00 min (SOLUTION A/SOLUTION B=90/10), 1.20 min (SOLUTION A/SOLUTION B=90/10), 7.75 min (SOLUTION A/SOLUTION B=50/50), 7.85 min (SOLUTION A/SOLUTION B=5/95), 8.85 min (SOLUTION A/SOLUTION B=5/95), 8.95 min (SOLUTION A/SOLUTION B=90/10)_Flow rate: 25 mL/min, Detection method: UV 220 nm]. The fraction containing the title compound was diluted with ethyl acetate (100 mL) and saturated aqueous sodium hydrogen carbonate solution (100 mL). The aqueous layer was extracted with ethyl acetate (100 mL), the combined organic layer was dried over anhydrous magnesium sulfate, and the insoluble material was filtered off. The filtrate was concentrated under reduced pressure, and the obtained solid was washed with diethyl ether (2 mL) and hexane (1 mL) to give the title compound (67 mg) as a colorless solid.

LC-MS 582.3 (MH⁺).

¹H NMR (DMSO-d₆, 300 MHz): δ 0.81-1.31 (8H, m), 1.48-2.24 (9H, m), 2.67-3.11 (4H, m), 3.24-3.61 (2H, m), 3.82-4.53 (4H, m), 4.90-5.00 (1H, m), 5.01-5.15 (1H, m), 5.66-6.11 (1H, m), 6.78 (1H, d, J=8.5 Hz), 6.82-6.98 (1H, m), 7.08-7.33 (4H, m), 7.36-7.54 (2H, m), 8.07-8.50 (1H, m), 8.53-8.81 (1H, m).

### Example 46

### Production of N-{(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}-N-2-methyl-L-alaninamide dihydrochloride

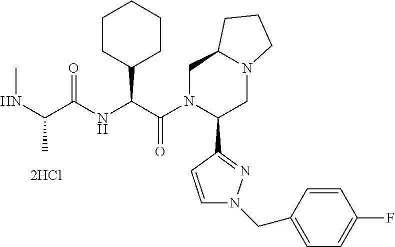

### (i) Production of tert-butyl(3S,8aR)-3-[methoxy(methyl)carbamoyl]hexahydropyrrolo[1,2-a]pyrazine-2(1H)-carboxylate

To a solution of 2-tert-butyl 3-methyl(3S,8aR)-hexahydropyrrolo[1,2-a]pyrazine-2,3(1H)-dicarboxylate (1.99 g) in THF (30 mL)/water (10 mL) was added lithium hydroxide monohydrate (441 mg), and the mixture was stirred at 50° C. for 3 hr, neutralized with 1M hydrochloric acid (10.5 mL), and concentrated under reduced pressure. The residue was diluted with DMF (20 mL), and N,O-dimethylhydroxylamine hydrochloride (1.37 g), 1-hydroxybenzotriazole (946 mg), N-[3-(dimethylamino)propyl]-N′-ethylcarbodiimide (5.37 g) and triethylamine (2.93 mL) were successively added thereto with stirring. The mixture was stirred at room temperature for 18 hr, and concentrated under reduced pressure. The residue was diluted with 5% aqueous sodium bicarbonate, and the mixture was extracted with ethyl acetate. The organic layer was concentrated under reduced pressure, and the residue was purified by silica gel column chromatography (ethyl acetate/methanol=100/0→90/10) to give the title compound (1.24 g). LC-MS: 314.2 (MH⁺).

¹H NMR (DMSO-d₆, 300 MHz, ca 1:1 rotamer mixture): δ 1.15-1.35 (1H, m), 1.35 (0.5×9H, s), 1.39 (0.5×9H, s), 1.55-1.85 (4H, m), 1.85-2.00 (1H, m), 2.20-2.35 (1H, m), 2.85-3.35 (3H, m), 3.09 (0.5×3H, s), 3.11 (0.5×3H, s), 3.69 (0.5×3H, s), 3.71 (0.5×3H, s), 3.80-3.90 (1H, m), 4.60-4.80 (1H, m).

### (ii) Production of tert-butyl(3S,8aR)-3-acetylhexahydropyrrolo[1,2-a]pyrazine-2(1H)-carboxylate

To a solution of tert-butyl(3S,8aR)-3-[methoxy(methyl)carbamoyl]hexahydropyrrolo[1,2-a]pyrazine-2(1H)-carboxylate (1.24 g) in THF (30 mL) was added methylmagnesium chloride (3M, 8 mL) with stirring under ice-cooling, and the mixture was stirred for 4 hr under ice-cooling. To the mixture was added 10% aqueous ammonium chloride solution (50 mL), and the mixture was extracted with ethyl acetate. The organic layer was concentrated under reduced pressure, and the residue was purified by silica gel column chromatography (hexane/ethyl acetate=50/50→0/100) to give the title compound (337 mg). LC-MS: 269.2 (MH⁺).

¹H NMR (DMSO-d₆, 300 MHz, ca 1:1 rotamer mixture): δ 1.10-1.30 (1H, m), 1.34 (0.5×9H, s), 1.40 (0.5×9H, s), 1.55-1.85 (4H, m), 1.90-2.05 (1H, m), 2.12 (0.5×3H, s), 2.13 (0.5×3H, s), 2.15-2.30 (1H, m), 2.40-2.70 (1H, m), 2.90-3.00 (1H, m), 3.50-3.60 (1H, m), 3.80-3.95 (1H, m), 4.45-4.65 (1H, m).

### (iii) Production of tert-butyl(3S,8aR)-3-(1H-pyrazol-3-yl)hexahydropyrrolo[1,2-a]pyrazine-2(1H)-carboxylate

tert-Butyl(3S,8aR)-3-acetylhexahydropyrrolo[1,2-a]pyrazine-2(1H)-carboxylate (333 mg) was dissolved in DMF (4 mL), tris(dimethylamino)methane (2.0 mL) was added thereto, and the mixture was stirred at 90° C. for 5 hr, and concentrated under reduced pressure. The residue was dissolved in ethanol (6 mL), hydrazine. (0.18 mL) was added thereto, and the mixture was stirred at 90° C. for 3 hr. The mixture was concentrated under reduced pressure, and the residue was purified by silica gel column chromatography (ethyl acetate/methanol=100/0→80/20) to give the title compound (246 mg). LC-MS: 293.2 (MH⁺).

### (iv) Production of tert-butyl(3S,8aR)-3-[1-(4-fluorobenzyl)-1H-pyrazol-3-yl]hexahydropyrrolo[1,2-a]pyrazine-2(1H)-carboxylate

To a solution of tert-butyl(3S,8aR)-3-(1H-pyrazol-3-yl)hexahydropyrrolo[1,2-a]pyrazine-2(1H)-carboxylate (246 mg) and 4-fluorobenzyl chloride (0.15 mL) in DMF (3 mL) was added potassium carbonate (349 mg), and the mixture was stirred at room temperature for 18 hr. 4-Fluorobenzyl chloride (0.15 mL), potassium carbonate (349 mg) and DMF (3 mL) were added thereto, and the mixture was stirred at room temperature for 30 hr. The mixture was diluted with ethyl acetate, and washed with water. The organic layer was concentrated under reduced pressure, and the residue was subjected to silica gel column chromatography (ethyl acetate/methanol=100/0→90/10). The object fractions were concentrated under reduced pressure, and the residue was purified by basic silica gel column chromatography (hexane/ethyl acetate=50/50→0/100) to give the title compound (135 mg). LC-MS: 401.3 (MH⁺).

¹H NMR (CDCl₃, 300 MHz): δ 1.15-2.15 (15H, m), 2.44 (1H, dd, J=10.8, 4.5 Hz), 2.70-2.95 (1H, m), 3.03 (1H, br t, J=8.3 Hz), 3.45-3.70 (1H, m), 4.00-4.25 (1H, m), 5.10-5.50 (3H, m), 6.00-6.20 (1H, m), 6.90-7.30 (5H, m).

### (v) Production of (3S,8aR)-3-[1-(4-fluorobenzyl)-1H-pyrazol-3-yl]octahydropyrrolo[1,2-a]pyrazine

tert-Butyl(3S,8aR)-3-[1-(4-fluorobenzyl)-1H-pyrazol-3-yl]hexahydropyrrolo[1,2-a]pyrazine-2(1H)-carboxylate (135 mg) was dissolved in 4M hydrogen chloride-ethyl acetate solution (20 mL), and the solution was stirred at room temperature for 1 hr. The mixture was concentrated under reduced pressure, and the residue was purified by basic silica gel column chromatography (ethyl acetate/methanol=100/0→70/30) to give the title compound (94 mg). LC-MS: 301.1 (MH⁺).

¹H NMR (CDCl₃, 300 MHz): δ 1.35-1.90 (4H, m), 2.10-2.35 (3H, m), 2.62 (1H, dd, J=11.0, 3.7 Hz), 2.80 (1H, dd, J=11.4, 8.4 Hz), 2.93-3.07 (2H, m), 3.19 (1H, dd, J=10.8, 3.6 Hz), 4.14 (1H, t, J=3.6 Hz), 5.23 (2H, s), 6.38 (1H, d, J=2.4 Hz), 6.96-7.06 (2H, m), 7.13-7.22 (2H, m), 7.25 (1H, d, J=2.4 Hz).

### (vi) Production of tert-butyl{(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}carbamate

To a solution of (3S,8aR)-3-[1-(4-fluorobenzyl)-1H-pyrazol-3-yl]octahydropyrrolo[1,2-a]pyrazine (92 mg), (2S)-[(tert-butoxycarbonyl)amino](cyclohexyl)ethanoic acid (126 mg) and N-ethyldiisopropylamine (0.213 mL) in DMF (3 mL) was added O-(7-azabenzotriazol-1-yl)-N,N,N′,N′-tetramethyluronium hexafluorophosphate (233 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was purified by basic silica gel column chromatography (hexane/ethyl acetate=95/5→50/50) to give the title compound (143 mg). LC-MS: 540.4 (MH⁺).

### (vii) Production of (1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethanamine

To a solution of tert-butyl{(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}carbamate (143 mg) in ethyl acetate (3 mL) was added 4M hydrogen chloride-ethyl acetate solution (12 mL), and the mixture was stirred at room temperature for 1 hr. The mixture was concentrated under reduced pressure, 5% aqueous sodium bicarbonate was added thereto, and the mixture was extracted with ethyl acetate. The organic layer was concentrated under reduced pressure, and the residue was purified by basic silica gel column chromatography (ethyl acetate/methanol=100/0→80/20) to give the title compound (108 mg). LC-MS: 440.3 (MH⁺).

### (viii) Production of tert-butyl[(1S)-2-({(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}amino)-1-methyl-2-oxoethyl]methylcarbamate

To a solution of (1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethanamine (108 mg), N-(tert-butoxycarbonyl)-N-methyl-L-alanine (75 mg) and N-ethyldiisopropylamine (0.171 mL) in DMF (3 mL) was added O-(7-azabenzotriazol-1-yl)-N,N,N′,N′-tetramethyluronium hexafluorophosphate (187 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was purified by basic silica gel column chromatography (hexane/ethyl acetate=95/5→0/100) to give the title compound (152 mg). LC-MS: 625.4 (MH⁺).

(ix) Production of N-{(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}-N²-methyl-L-alaninamide dihydrochloride

To a solution of tert-butyl[(1S)-2-({(1S)-1-cyclohexyl-2-[(3S,8aR)-3-[1-(4-fluorobenzyl)-1H-pyrazol-3-yl]hexahydropyrrolo[1,2-a]pyrazin-2(1H)-yl]-2-oxoethyl}amino)-1-methyl-2-oxoethyl]methylcarbamate (152 mg) in ethyl acetate (5 mL) was added 4M hydrogen chloride-ethyl acetate solution (20 mL), and the mixture was stirred at room temperature for 1 hr. The mixture was concentrated under reduced pressure, 5%, aqueous sodium bicarbonate was added thereto, and the mixture was extracted with ethyl acetate. The organic layer was concentrated under reduced pressure, and the residue was subjected to basic silica gel column chromatography (ethyl acetate/methanol=100/0→80/20). The object fractions were concentrated under reduced pressure, the residue was diluted with ethyl acetate (5 mL), and 4M hydrogen chloride-ethyl acetate solution (1 mL) was added thereto. The precipitate was collected by filtration, washed with ethyl acetate, and dried under reduced pressure to give the title compound (117 mg). LC-MS: 525.4 (MH⁺).

¹H NMR (DMSO-d₆, 300 MHz): δ 0.80-2.25 (18H, m), 2.35-2.55 (3H, m), 2.60-4.95 (9H, m), 5.15-6.10 (3H, m), 6.10-6.30 (1H, m), 7.10-7.40 (4H, m), 7.75-7.90 (1H, m), 8.35-9.00 (2H, m), 9.15-9.70 (1H, m), 11.80-12.30 (1H, m).

### Example 47

### Production of N-{(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}-N²-methyl-L-alaninamide dihydrochloride

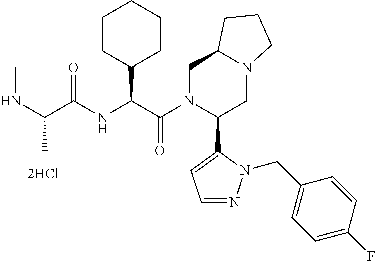

### (i) Production of tert-butyl(3S,8aR)-3-[1-(4-fluorobenzyl)-1H-pyrazol-5-yl]hexahydropyrrolo[1,2-a]pyrazine-2(1H)-carboxylate

tert-Butyl(3S,8aR)-3-acetylhexahydropyrrolo[1,2-a]pyrazine-2(1H)-carboxylate (710 mg) was dissolved in DMF (4 mL), tris(dimethylamino)methane (2.0 mL) was added thereto, and the mixture was stirred at 90° C. for 3 hr. The mixture was concentrated under reduced pressure, and the residue was dissolved in ethanol (10 mL). (4-Fluorobenzyl)hydrazine hydrochloride (562 mg) was added thereto, and the mixture was stirred at 60° C. for 3 hr. The mixture was concentrated under reduced pressure, the residue was diluted with ethyl acetate, and the mixture was washed with 5% aqueous sodium bicarbonate.

The organic layer was concentrated under reduced pressure, and the residue was purified by silica gel column chromatography (hexane/ethyl acetate=95/5→0/100) to give the title compound (704 mg). LC-MS: 401.2 (MH⁺).

¹H NMR (CDCl₃, 300 MHz): δ 1.15-2.10 (15H, m), 2.38 (1H, dd, J=11.1, 4.2 Hz), 2.55-2.70 (1H, m), 2.95-3.15 (2H, m), 3.80-4.20 (1H, m), 5.15-5.60 (3H, m), 6.60-6.75 (1H, m), 6.85-7.15 (4H, m), 7.45 (1H, d, J=1.2 Hz).

### (ii) Production of (3S,8aR)-3-[1-(4-fluorobenzyl)-1H-pyrazol-5-yl]octahydropyrrolo[1,2-a]pyrazine dihydrochloride

To a solution of tert-butyl(3S,8aR)-3-[1-(4-fluorobenzyl)-1H-pyrazol-5-yl]hexahydropyrrolo[1,2-a]pyrazine-2(1H)-carboxylate (700 mg) in ethyl acetate/methanol (5 mL/5 mL) was added 4M hydrogen chloride-ethyl acetate solution (30 mL), and the mixture was stirred at room temperature for 1 hr. The mixture was concentrated under reduced pressure, and ethyl acetate was added to the residue. The precipitate was collected by filtration, washed with ethyl acetate, and dried under reduced pressure to give the title compound (630 mg). LC-MS: 301.2 (MH⁺).

¹H NMR (CD₃OD, 300 MHz): δ 2.08-2.56 (4H, m), 3.27-3.44 (1H, m), 3.59-3.82 (3H, m), 3.82-4.05 (2H, m), 4.12-4.26 (1H, m), 5.25 (1H, dd, J=11.5, 3.5 Hz), 5.52 & 5.64 (2H, ABq, J=16.0 Hz), 6.92 (1H, d, J=2.1 Hz), 7.04-7.13 (2H, m), 7.31-7.40 (2H, m), 7.68 (1H, d, J=2.1 Hz).

### (iii) Production of tert-butyl{(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}carbamate

To a solution of (3S,8aR)-3-[1-(4-fluorobenzyl)-1H-pyrazol-5-yl]octahydropyrrolo[1,2-a]pyrazine dihydrochloride (299 mg), (2S)-[(tert-butoxycarbonyl)amino](cyclohexyl)ethanoic acid (309 mg) and N-ethyldiisopropylamine (0.836 mL) in DMF (6 mL) was added O-(7-azabenzotriazol-1-yl)-N,N,N′,N′-tetramethyluronium hexafluorophosphate (608 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was purified by basic silica gel column chromatography (hexane/ethyl acetate=97/3→50/50) to give the title compound (338 mg). LC-MS: 540.3 (MH⁺).

¹H NMR (CDCl₃, 300 MHz): δ 0.82-2.12 (17H, m), 1.44 (9H, s), 2.35 (1H, dd, J=11.4, 4.5 Hz), 2.93-3.12 (3H, m), 3.87 (1H, br d, J=12.9 Hz), 4.46 (1H, dd, J=9.3, 5.4 Hz), 5.29 (1H, d, J=9.3 Hz), 5.33 & 5.55 (2H, ABq, J=15.5 Hz), 5.87 (1H, d, J=3.9 Hz), 6.79 (1H, d, J=1.8 Hz), 6.93-7.03 (2H, m), 7.10-7.18 (2H, m), 7.45 (1H, d, J=1.8 Hz).

### (iv) Production of (1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethanamine

To a solution of tert-butyl{(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}carbamate (335 mg) in ethyl acetate (5 mL) was added 4M hydrogen chloride-ethyl acetate solution (25 mL), and the mixture was stirred at room temperature for 1 hr. The mixture was concentrated under reduced pressure, 5% aqueous sodium bicarbonate was added thereto, and the mixture was extracted with ethyl acetate. The organic layer was concentrated under reduced pressure, and the residue was purified by basic silica gel column chromatography (ethyl acetate/methanol=100/0→80/20) to give the title compound (275 mg). LC-MS: 440.3 (MH⁺).

¹H NMR (CDCl₃, 300 MHz): δ 0.84-2.12 (19H, m), 2.37 (1H, dd, J=11.3, 4.4 Hz), 2.98-3.09 (3H, m), 3.49 (1H, d, J=5.1 Hz), 3.76 (1H, br d, J=13.1 Hz), 5.33 & 5.59 (2H, ABq, J=15.6 Hz), 5.93 (1H, d, J=3.9 Hz), 6.79 (1H, d, J=1.8 Hz), 6.93-7.02 (2H, m), 7.13-7.21 (2H, m), 7.45 (1H, d, J=1.8 Hz).

### (v) Production of tert-butyl[(1S)-2-({(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}amino)-1-methyl-2-oxoethyl]methylcarbamate

To a solution of (1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethanamine (272 mg), N-(tert-butoxycarbonyl)-N-methyl-L-alanine (189 mg) and N-ethyldiisopropylamine (0.431 mL) in DMF (6 mL) was added O-(7-azabenzotriazol-1-yl)-N,N,N′,N′-tetramethyluronium hexafluorophosphate (471 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was subjected to basic silica gel column chromatography (hexane/ethyl acetate=99/1→50/50). The object fractions were concentrated under reduced pressure, and the residue was purified by silica gel column chromatography (hexane/ethyl acetate=90/10→0/100) to give the title compound (371 mg). LC-MS: 625.4 (MH⁺).

¹H NMR (CDCl₃, 300 MHz): δ 0.77-2.10 (17H, m), 1.34 (3H, d, J=7.2 Hz), 1.51 (9H, s), 2.34 (1H, dd, J=11.3, 4.1 Hz), 2.81 (3H, s), 2.91-3.13 (3H, m), 3.89 (1H, br d, J=12.6 Hz), 4.51-4.91 (2H, m), 5.33 & 5.57 (2H, ABq, J=15.6 Hz), 5.85 (1H, d, J=3.9 Hz), 6.68-6.88 (1H, m), 6.79 (1H, d, J=1.8 Hz), 6.93-7.02 (2H, m), 7.11-7.19 (2H, m), 7.45 (1H, d, J=1.8 Hz).

### (vi) Production of N-{(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}-N²-methyl-L-alaninamide dihydrochloride

To a solution of tert-butyl[(1S)-2-({(1S)-1-cyclohexyl-2-[(3S,8aR)-3-[1-(4-fluorobenzyl)-1H-pyrazol-5-yl]hexahydropyrrolo[1,2-a]pyrazin-2(1H)-yl]-2-oxoethyl}amino)-1-methyl-2-oxoethyl]methylcarbamate (371 mg) in ethyl acetate (5 mL) was added 4M hydrogen chloride-ethyl acetate solution (25 mL), and the mixture was stirred at room temperature for 2 hr. The mixture was concentrated under reduced pressure, ethyl acetate was added to the residue, and the precipitate was collected by filtration, washed with ethyl acetate, and dried under reduced pressure to give the title compound (320 mg). LC-MS: 525.4).

¹H NMR (DMSO-d₆, 300 MHz): δ 0.79-2.24 (15H, m), 1.39 (3H, d, J=6.6 Hz), 2.40-2.50 (3H, m), 2.89-4.09 (7H, m), 4.42-4.57 (1H, m), 4.78 (1H, t, J=7.5 Hz), 5.31-5.59 (3H, m), 6.36 (1H, J=1.8 Hz), 7.09-7.20 (2H, m), 7.36-7.45 (3H, m), 8.76 (1H, d, J=8.4 Hz), 8.78-8.98 (1H, m), 9.25-9.42 (1H, m), 11.99-12.17 (1H, m).

### Example 48

### Production of (3S,8aS)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

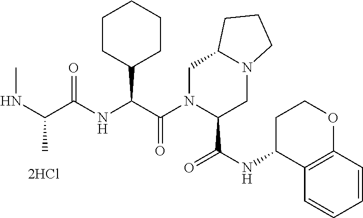

### (i) Production of tert-butyl{(1S)-1-cyclohexyl-2-[(3S,8aS)-3-[(4R)-3,4-dihydro-2H-chromen-4-ylcarbamoyl]hexahydropyrrolo[1,2-a]pyrazin-2(1H)-yl]-2-oxoethyl}carbamate

To a solution of (3S,8aS)—N-[(4R)-3,4-dihydro-2H-chromen-4-yl]octahydropyrrolo[1,2-a]pyrazine-3-carboxamide dihydrochloride (343 mg), (2S)-[(tert-butoxycarbonyl)amino](cyclohexyl)ethanoic acid (355 mg) and N-ethyldiisopropylamine (0.96 mL) in DMF (6 mL) was added O-(7-azabenzotriazol-1-yl)-N,N,N′,N′-tetramethyluronium hexafluorophosphate (699 mg), and the mixture was stirred at room temperature for 18-hr. The mixture was diluted with ethyl acetate, and washed successively with water, 5% aqueous sodium bicarbonate and water. The organic layer was concentrated under reduced pressure, and the residue was subjected to silica gel column chromatography (ethyl acetate/methanol=100/0→50/50). The object fractions were concentrated under reduced pressure, and the residue was purified by basic silica gel column chromatography (hexane/ethyl acetate/methanol=90/10/0→0/100/0→0/70/30) to give the title compound (428 mg). LC-MS: 541.4 (MH⁺).

### (ii) Production of (3S,8aS)-2-[(2S)-2-amino-2-cyclohexylacetyl]-N-[(4R)-3,4-dihydro-2H-chromen-4-yl]octahydropyrrolo[1,2-a]pyrazine-3-carboxamide dihydrochloride

To a solution of tert-butyl{(1S)-1-cyclohexyl-2-[(3S,8aS)-3-[(4R)-3,4-dihydro-2H-chromen-4-ylcarbamoyl]hexahydropyrrolo[1,2-a]pyrazin-2(1H)-yl]-2-oxoethyl}carbamate (425 mg) in ethyl acetate (5 mL) was added 4M hydrogen chloride-ethyl acetate solution (25 mL), and the to mixture was stirred at room temperature for 2 hr. The mixture was concentrated under reduced pressure, ethyl acetate was added to the residue, and the precipitate was collected by filtration, washed with ethyl acetate, and dried under reduced pressure to give the title compound (345 mg). LC-MS: 441.3 (MH⁺).

### (iii) Production of tert-butyl[(1S)-2-({(1S)-1-cyclohexyl-2-[(3S,8aS)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]methylcarbamate

To a solution of (3S,8aS)-2-[(2S)-2-amino-2-cyclohexylacetyl]-N-[(4R)-3,4-dihydro-2H-chromen-4-yl]octahydropyrrolo[1,2-a]pyrazine-3-carboxamide dihydrochloride (342 mg), N-(tert-butoxycarbonyl)-N-methyl-L-alanine (203 mg) and N-ethyldiisopropylamine (0.696 mL) in DMF (6 mL) was added O-(7-azabenzotriazol-1-yl)-N,N,N′,N′-tetramethyluronium hexafluorophosphate (506 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was subjected to silica gel column chromatography (ethyl acetate/methanol=100/0→30/70). The object fractions were concentrated under reduced pressure, and the residue was purified by basic silica gel column chromatography (ethyl acetate/methanol=100/0→70/30) to give the title compound (341 mg). LC-MS: 626.4 (MH⁺).

### (iv) Production of (3S,8aS)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide dihydrochloride

To a solution of tert-butyl[(1S)-2-({(1S)-1-cyclohexyl-2-[(3S,8aS)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]methylcarbamate (338 mg) in ethyl acetate (5 mL) was added 4M hydrogen chloride-ethyl acetate solution (25 mL), and the mixture was stirred at room temperature for 2 hr. The mixture was concentrated under reduced pressure, ethyl acetate was added to the residue, and the precipitate was collected by filtration, washed with ethyl acetate, and dried under reduced pressure to give the title compound (278 mg). LC-MS: 526.4 (MH⁺).

¹H NMR (DMSO-d₆, 300 MHz): δ 0.90-2.30 (20H, m), 2.40-2.50 (3H, m), 2.95-5.10 (13H, m), 6.70-6.95 (2H, m), 7.10-7.60 (2H, m), 8.60-9.00 (3H, m), 9.25-9.60 (1H, m), 11.00-11.40 (1H, m).

### Example 49

### Production of (3S,8aR)-2-{(2S)-2-cyclohexyl-2-[(N-ethyl-L-alanyl)amino]acetyl}-N-[(4R)-3,4-dihydro-2H-chromen-4-yl]octahydropyrrolo[1,2-a]pyrazine-3-carboxamide dihydrochloride

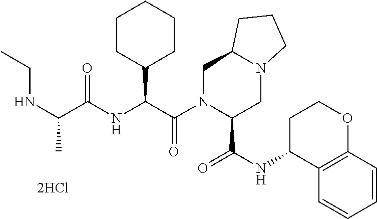

### (i) Production of methyl(3S,8aR)-2-[(2S)-2-{[N-(tert-butoxycarbonyl)-N-ethyl-L-alanyl]amino}-2-cyclohexylacetyl]octahydropyrrolo[1,2-a]pyrazine-3-carboxylate

To a solution of methyl(3S,8aR)-2-[(2S)-2-amino-2-cyclohexylacetyl]octahydropyrrolo[1,2-a]pyrazine-3-carboxylate dihydrochloride (793 mg), N-(tert-butoxycarbonyl)-N-ethyl-L-alanine (652 mg) and N-ethyldiisopropylamine (2.09 mL) in DMF (10 mL) was added O-(7-azabenzotriazol-1-yl)-N,N,N′,N′-tetramethyluronium hexafluorophosphate (1521 mg), and the mixture was stirred at room temperature for 18 hr. The mixture was diluted with ethyl acetate, and washed successively with water, 5% aqueous sodium bicarbonate and water. The organic layer was concentrated under reduced pressure, and the residue was subjected to silica gel column chromatography (hexane/ethyl acetate/methanol=50/50/0→0/100/0→0/80/20). The object fractions were concentrated under reduced pressure, and the residue was purified by basic silica gel column chromatography (hexane/ethyl acetate=90/10→0/100) to give the title compound (304 mg). LC-MS: 523.4 (MH⁺).

### (ii) Production of tert-butyl[(1S)-2-({(1S)-1-cyclohexyl-2-[(3S,8aR)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]ethylcarbamate

To a solution of methyl(3S,8aR)-2-[(2S)-2-{[N-(tert-butoxycarbonyl)-N-ethyl-L-alanyl]amino}-2-cyclohexylacetyl]octahydropyrrolo[1,2-a]pyrazine-3-carboxylate (300 mg) in THF (3 mL)/water (1 mL) was added lithium hydroxide hydrate (36 mg), and the mixture was stirred at 50° C. for 4 hr, neutralized with 1N hydrochloric acid (0.86 mL), and concentrated under reduced pressure. To the residue was added toluene, and the mixture was concentrated under reduced pressure, and dried under reduced pressure. The residue was diluted with DMF (6 mL), (4R)-3,4-dihydro-2H-chromen-4-amine hydrochloride (138 mg), N-ethyldiisopropylamine (0.596 mL) and O-(7-azabenzotriazol-1-yl)-N,N,N′,N′-tetramethyluronium hexafluorophosphate (433 mg) were successively added thereto, and the mixture was stirred at room temperature for 18 hr. The mixture was diluted with ethyl acetate, and washed successively with water, 5% aqueous sodium bicarbonate and water. The organic layer was concentrated under reduced pressure, and the residue was subjected to silica gel column chromatography (ethyl acetate/methanol=100/0→70/30). The object fractions were concentrated under reduced pressure, and the residue was purified by basic silica gel column chromatography (hexane/ethyl acetate=90/10→0/100) to give the title compound (111 mg). LC-MS: 640.4 (MH⁺).

### (iii) Production of (3S,8aR)-2-{(2S)-2-cyclohexyl-2-[(N-ethyl-L-alanyl)amino]acetyl}-N-[(4R)-3,4-dihydro-2H-chromen-4-yl]octahydropyrrolo[1,2-a]pyrazine-3-carboxamide dihydrochloride

To a solution of tert-butyl[(1S)-2-({(1S)-1-cyclohexyl-2-[(3S,8aR)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]ethylcarbamate (111 mg) in ethyl acetate (5 mL) was added 4M hydrogen chloride-ethyl acetate solution (25 mL), and the mixture was stirred at room temperature for 2 hr, and concentrated under reduced pressure. To the residue was added a mixed solvent of ethyl acetate/hexane (1/1), and the precipitate was collected by filtration, washed with a mixed solvent of ethyl acetate/hexane (1/1), and dried under reduced pressure to give the title compound (88 mg). LC-MS: 540.4 (MH⁺).

¹H NMR (CD₃OD, 300 MHz): δ 1.00-2.40 (23H, m), 2.90-5.25 (15H, m), 6.70-6.95 (2H, m), 7.10-7.30 (2H, m).

### Example 50

### Production of (3S,8aR)-2-{(2S)-2-cyclohexyl-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide dihydrochloride

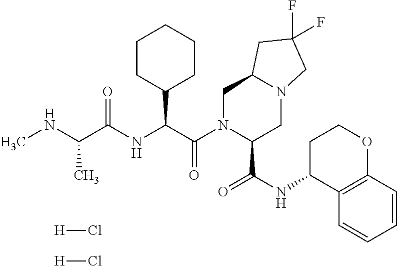

### (i) Production of 2-tert-butyl 3-methyl(3S,8aR)-7-oxohexahydropyrrolo[1,2-a]pyrazine-2,3(1H)-dicarboxylate

To a solution of 2-tert-butyl 3-methyl(3S,7R,8aR)-7-hydroxyhexahydropyrrolo[1,2-a]pyrazine-2,3(1H)-dicarboxylate (1.62 g) in dimethylsulfide (3.24 mL)/ethyl acetate (8.1 mL) were added dropwise triethylamine (4.5 mL) and a solution of sulfur trioxide pyridine complex (2.58 g) in dimethylsulfide (13.0 mL) under ice-cooling. The mixture was stirred for 1 hr under ice-cooling, and poured into ice water, and extracted with a mixed solvent of ethyl acetate/tetrahydrofuran (2/1). The extract was washed with saturated aqueous sodium hydrogen carbonate and saturated brine, dried over anhydrous magnesium sulfate, filtered, and concentrated under reduced pressure. The residue was purified by silica gel column chromatography (hexane/ethyl acetate=70/30→20/80). The object fractions were collected, the solvent was evaporated under reduced pressure, and the residue was dried in vacuum to give the title compound (1.46 g) as a colorless oil.

¹H NMR (DMSO-d₆, 300 MHz): δ 1.34-1.45 (9H, m), 2.02-2.14 (1H, m), 2.30-2.40 (1H, m), 2.45-2.62 (2H, m), 2.62-2.71 (1H, m), 2.73-3.00 (1H, m), 3.24-3.35 (1H, m), 3.40-3.48 (1H, m), 3.66-3.70 (3H, m), 3.90-4.03 (1H, m), 4.70 (1H, dd, J=19.9, 3.4 Hz).

### (ii) Production of 2-tert-butyl 3-methyl(3S,8aR)-7,7-difluorohexahydropyrrolo[1,2-a]pyrazine-2,3(1H)-dicarboxylate

To a solution of 2-tert-butyl 3-methyl(3S,8aR)-7-oxohexahydropyrrolo[1,2-a]pyrazine-2,3(1H)-dicarboxylate (2.8 g) in toluene (28 mL) was added dropwise bis(2-methoxyethyl)aminosulfur trifluoride under ice-cooling. The mixture was stirred for 1 hr under ice-cooling, and then at room temperature for 20 hr. The reaction mixture was poured into ice-cooled saturated aqueous sodium hydrogen carbonate, and extracted with ethyl acetate. The extract was washed with saturated brine, dried over anhydrous magnesium sulfate, and filtered. The filtrate was concentrated under reduced pressure, and the residue was purified by silica gel column chromatography (hexane/ethyl acetate→80/20→40/60). The object is fractions were collected, and concentrated under reduced pressure to give the title compound (1.73 g) as a colorless oil. LC-MS: 321.1 (MH⁺)

¹H NMR (DMSO-d₆, 300 MHz): δ 1.33-1.44 (9H, m), 1.75-2.00 (1H, m), 2.27-2.60 (4H, m), 2.61-2.89 (1H, m), 3.30-3.43 (2H, m), 3.65-3.70 (3H, m), 3.87-3.98 (1H, m), 4.67 (1H, dd, J=21.7, 3.4 Hz).

### (iii) Production of methyl(3S,8aR)-2-{(2S)-2-[(tert-butoxycarbonyl)amino]-2-cyclohexylacetyl}-7,7-difluorooctahydropyrrolo[1,2-a]pyrazine-3-carboxylate

To a solution of 2-tert-butyl 3-methyl(3S,8aR)-7,7-difluorohexahydropyrrolo[1,2-a]pyrazine-2,3(1H)-dicarboxylate (1.0 g) in ethyl acetate (5 mL) was added 4M hydrogen chloride-ethyl acetate solution (30 mL), and the mixture was stirred at room temperature for 1 hr, and concentrated under reduced pressure. To the residue was added ethyl acetate, and the mixture was stirred at room temperature for 30 min. The precipitate was collected by filtration, washed with ethyl acetate, and dried in vacuum. To the obtained residue was added N,N-dimethylformamide (15 mL), N,N-diisopropylethylamine (4.35 mL), (2S)-[(tert-butoxycarbonyl)amino](cyclohexyl)ethanoic acid (1.20 g) and O-(7-azabenzotriazol-1-yl)-N,N,N′,N′-tetramethyluronium hexafluorophosphate (2.37 g) were added thereto, and the mixture was stirred at room temperature for 18 hr. To the reaction mixture were added ethyl acetate and saturated aqueous sodium hydrogen carbonate, and the mixture was extracted with ethyl acetate. The extract was washed with saturated brine, dried over anhydrous magnesium sulfate, and filtered. The filtrate was concentrated under'reduced pressure, and the residue was purified by silica gel column chromatography (hexane/ethyl acetate=70/30→40/60). The object fractions were collected, and concentrated under reduced is pressure. The residue was purified by basic silica gel column chromatography (hexane/ethyl acetate=70/30→20/80). The object fractions were collected, and concentrated under reduced pressure, and the residue was dried in vacuum for 1 hr to give the title compound (636 mg) as a white powder.

LC-MS: 460 (MH⁺).

¹H NMR (DMSO-d₆, 300 MHz): δ 0.80-1.25 (5H, m), 1.26-1.40 (9H, m), 1.53-1.75 (6H, m), 1.80-2.05 (1H, m), 2.15-2.65 (4H, m), 2.93-3.07 (1H, m), 3.25-3.50 (2H, m), 3.61-3.68 (3H, m), 4.12-4.29 (1H, m), 4.31-4.43 (1H, m), 5.15-5.26 (1H, m), 6.74-6.87 (1H, m).

### (iv) Production of methyl(3S,8aR)-2-[(2S)-2-{[N-(tert-butoxycarbonyl)-N-methyl-L-alanyl]amino}-2-cyclohexylacetyl]-7,7-difluorooctahydropyrrolo[1,2-a]pyrazine-3-carboxylate

To methyl(3S,8aR)-2-{(2S)-2-[(tert-butoxycarbonyl)amino]-2-cyclohexylacetyl}-7,7-difluorooctahydropyrrolo[1,2-a]pyrazine-3-carboxylate (634 mg) were added ethyl acetate (3.2 mL) and 4M hydrogen chloride-ethyl acetate solution (19.0 mL), and the mixture was stirred at room temperature for 1 hr, and concentrated under reduced pressure. To the residue was added ethyl acetate, and the precipitate was collected by filtration, washed with ethyl acetate, and dried in vacuum. To a solution of the obtained powder in N,N-dimethylformamide (9.5 mL) were added N,N-diisopropylethylamine (1.92 mL), N-(tert-butoxycarbonyl)-N-methyl-L-alanine (420 mg) and O-(7-azabenzotriazol-1-yl)-N,N,N′,N′-tetramethyluronium hexafluorophosphate (1.05 g), and the mixture was stirred at room temperature for 18 hr. Water was added to the reaction mixture, and the mixture was extracted with ethyl acetate. The extract was washed with saturated aqueous sodium hydrogen carbonate and saturated brine, dried over anhydrous magnesium sulfate, and filtered. The filtrate was concentrated under reduced pressure, and the residue was purified by silica gel column chromatography (hexane/ethyl acetate=70/30→40/60). The object fractions were collected, and concentrated under reduced pressure, and the residue was dried in vacuum to give the title compound (400 mg) as a white powder.

¹H NMR (DMSO-d₆, 300 MHz): δ 0.75-1.27 (8H, m), 1.29-1.47 (9H, m), 1.52-1.80 (6H, m), 1.81-2.05 (1H, m), 2.15-2.65 (4H, m), 2.70-2.80 (3H, m), 2.87-3.50 (3H, m), 3.60-3.69 (3H, m), 4.15-4.78 (3H, m), 5.15-5.25 (1H, m), 7.50-7.90 (1H, m).

### (v) Production of tert-butyl[(1S)-2-({(1S)-1-cyclohexyl-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}amino)-1-methyl-2-oxoethyl]methylcarbamate

To a solution of methyl(3S,8aR)-2-[(2S)-2-{[N-(tert-butoxycarbonyl)-N-methyl-L-alanyl]amino}-2-cyclohexylacetyl]-7,7-difluorooctahydropyrrolo[1,2-a]pyrazine-3-carboxylate (195 mg) in tetrahydrofuran (5.85 mL)/water (1.37 mL) was added lithium hydroxide monohydrate (24.0 mg), and the mixture was stirred at 50° C. for 5 hr. The mixture was allowed to cool to room temperature, 1M hydrochloric acid (0.58 mL) was added thereto, and the mixture was concentrated under reduced pressure. To the residue was added toluene, the mixture was concentrated under reduced pressure, and the residue was dried in vacuum. To the obtained residue were added successively N,N-dimethylformamide (3.9 mL), (4R)-3,4-dihydro-2H-chromen-4-amine hydrochloride (100 mg), N,N-diisopropylethylamine (125 μl), 1-hydroxybenzotriazole (53 mg) and 1-ethyl-3-(3-dimethylaminopropyl)carbodiimide hydrochloride (412 mg), and the mixture was stirred at room temperature for 18 hr. Water was added to the reaction mixture, and the mixture was extracted with ethyl acetate. The extract was washed with saturated aqueous sodium hydrogen carbonate and saturated brine, dried over anhydrous magnesium sulfate, and filtered. The filtrate was concentrated under reduced pressure, and the residue was purified by basic silica gel column chromatography (hexane/ethyl acetate=33/67→0/100). The object fractions were collected, and concentrated under reduced pressure. The residue was purified by silica gel column chromatography (hexane/ethyl acetate=60/40→5/95). The object fractions were collected, and concentrated under reduced pressure, and the residue was dried in vacuum for 1 hr to give the title compound (80 mg) as a white powder.

LC-MS: 662.4 (MH⁺).

¹H NMR (DMSO-d₆, 300 MHz): δ 0.80-1.28 (9H, m), 1.29-1.47 (9H, m); 1.49-1.79 (6H, m), 1.80-2.09 (2H, m), 2.13-2.59 (4H, m), 2.59-2.80 (4H, m), 3.22-3.67 (2H, m), 3.98-5.10 (7H, m), 6.71-6.90 (2H, m), 7.09-7.25 (2H, m), 7.60-7.90 (1H, m), 8.18-8.35 (1H, m).

### (vi) Production of (3S,8aR)-2-{(2S)-2-cyclohexyl-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide dihydrochloride

To a suspension of tert-butyl[(1S)-2-({(1S)-1-cyclohexyl-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}amino)-1-methyl-2-oxoethyl]methylcarbamate (78 mg) in ethyl acetate (0.78 mL) was added 4M hydrogen chloride-ethyl acetate solution (3.9 mL), and the mixture was stirred at room temperature for 2 hr, and concentrated under reduced pressure. To the residue was added ethyl acetate, and the mixture was stirred at room temperature for 1 hr. The precipitate was collected by filtration, washed with ethyl acetate, and dried in vacuum to give the title compound (52 mg) as a white powder.

LC-MS: 562.4 (MH⁺).

¹H NMR (DMSO-d₆, 300 MHz): δ 0.91-1.29 (5H, m), 1.29-1.40 (3H, m), 1.48-2.11 (9H, m), 2.20-2.88 (9H, m), 3.28-5.09 (9H, m), 6.74-6.92 (2H, m), 7.07-7.29 (2H, m), 8.20-8.96 (3H, m), 9.18-9.38 (1H, m).

### Example 51

### Production of (1S,2S)-1-({[(3S,8aR)-2-{(2S)-2-cyclohexyl-2-[(N-methyl-L-alanyl)amino]acetyl}-7,7-difluorooctahydropyrrolo[1,2-a]pyrazin-3-yl]carbonyl}amino)-2,3-dihydro-1H-inden-2-yl acetate dihydrochloride

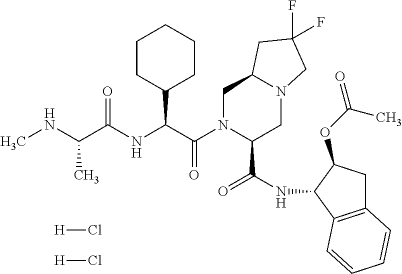

### (i) Production of tert-butyl[(1S)-2-({(1S)-1-cyclohexyl-2-[(3S,8aR)-7,7-difluoro-3-{[(1S,2S)-2-hydroxy-2,3-dihydro-1H-inden-1-yl]carbamoyl}hexahydropyrrolo[1,2-a]pyrazin-2(1H)-yl]-2-oxoethyl}amino)-1-methyl-2-oxoethyl]methylcarbamate

To a solution of methyl(3S,8aR)-2-[(2S)-2-{[N-(tert-butoxycarbonyl)-N-methyl-L-alanyl]amino}-2-cyclohexylacetyl]-7,7-difluorooctahydropyrrolo[1,2-a]pyrazine-3-carboxylate (195 mg) in tetrahydrofuran (6.0 mL)/water (1.4 mL) was added lithium hydroxide monohydrate (24.0 mg) at room temperature, and the reaction mixture was stirred at 50° C. for 5 hr. The reaction mixture was allowed to cool to room temperature, neutralized with 1M hydrochloric acid (0.58 mL), and concentrated under reduced pressure. The residue was subjected to azeotropic distillation with toluene, and dried in vacuum for 1 hr. The obtained residue was dissolved in N,N-dimethylformamide (3.9 mL), (1S,2S)-1-amino-2,3-dihydro-1H-inden-2-ol (80 mg), N,N-diisopropylethylamine (0.125 mL), 1-hydroxybenzotriazole (53 mg) and 1-ethyl-3-3-dimethylaminopropyl)carbodiimide hydrochloride (412 mg) were added thereto, and the mixture was stirred at room temperature for 18 hr. To the reaction mixture were added ethyl acetate and water, and the mixture was extracted with ethyl acetate. The extract was washed with saturated aqueous sodium hydrogen carbonate and saturated brine, dried over anhydrous magnesium sulfate, and filtered. The filtrate was concentrated under reduced pressure, and the residue was purified by basic silica gel column chromatography (hexane/ethyl acetate=33/67→0/100) and then silica gel column chromatography (hexane/ethyl acetate=60/40→5/95). The object fractions were collected, and concentrated under reduced pressure, and the residue was dried in vacuum to give the title compound (184 mg) as a white powder.

LC-MS: 662.4 (MH⁺).

¹H NMR (DMSO-d₆, 300 MHz): δ 0.80-1.29 (9H, m), 1.29-1.48 (9H, m), 1.52-2.04 (8H, m), 2.17-2.77 (6H, m), 3.06-3.18 (1H, m), 3.24-3.67 (3H, m), 3.98-5.07 (6H, m), 5.13-5.32 (1H, m), 7.03-7.23 (4H, m), 7.58-7.90 (1H, m), 8.09-8.27 (1H, m).

### (ii) Production of (1S,2S)-1-({[(3S,8aR)-2-{(2S)-2-cyclohexyl-2-[(N-methyl-L-alanyl)amino]acetyl}-7,7-difluorooctahydropyrrolo[1,2-a]pyrazin-3-yl]carbonyl}amino)-2,3-dihydro-1H-inden-2-yl acetate dihydrochloride

To a solution of tert-butyl[(1S)-2-({(1S)-1-cyclohexyl-2-[(3S,8aR)-7,7-difluoro-3-{[(1S,2S)-2-hydroxy-2,3-dihydro-1H-inden-1-yl]carbamoyl}hexahydropyrrolo[1,2-a]pyrazin-2(1H)-yl]-2-oxoethyl}amino)-1-methyl-2-oxoethyl]methylcarbamate (180 mg) in ethyl acetate (1.8 mL) was added 4M hydrogen chloride-ethyl acetate solution (9.0 mL), and the mixture was stirred at room temperature for 2 hr, and concentrated under reduced pressure. To the obtained residue was added ethyl acetate, and the mixture was stirred at room temperature for 1 hr. The precipitate was collected by filtration, washed with ethyl acetate, and dried in vacuum. To the obtained residue were added ethyl acetate (1.8 mL) and 4M hydrogen chloride-ethyl acetate solution (9.0 mL), and the mixture was stirred at room temperature for 16 hr, and concentrated under reduced pressure. To the residue were added ethyl acetate and saturated aqueous sodium hydrogen carbonate, and the mixture was extracted with ethyl acetate. The extract was washed with saturated brine, dried over anhydrous magnesium sulfate, and filtered. The filtrate was concentrated under reduced pressure, and the residue was purified by silica gel column chromatography (ethyl acetate/methanol=99/1→85/15). The object fractions were collected, and the solvent was evaporated under reduced pressure. To the obtained residue were added ethyl acetate (0.9 mL) and 4M hydrogen chloride-ethyl acetate solution (9.0 mL), and the mixture was stirred at room temperature for 30 min, and concentrated under reduced pressure. To the residue was added diethyl ether, and the mixture was stirred at room temperature for 30 min. The precipitate was collected by filtration, washed with diethyl ether, and dried in vacuum to give the title compound (112 mg) as a white powder.

LC-MS: 604.4 (MH⁺).

¹H NMR (DMSO-d₆, 300 MHz): δ 0.91-1.05 (5H, m), 1.05-1.40 (3H, m), 1.52-1.88 (6H, m), 1.97-2.07 (3H, m), 2.02-2.92 (8H, m), 3.26-5.41 (12H, m), 7.07-7.29 (4H, m), 8.32-8.91 (3H, m), 9.10-9.28 (1H, m).

### Example 52

### Production of (3S,8aR)-2-{(2S)-2-cyclohexyl-2-[(N-methyl-L-alanyl)amino]acetyl}-7,7-difluoro-N-[(1S,2S)-2-hydroxy-2,3-dihydro-1H-inden-1-yl]octahydropyrrolo[1,2-a]pyrazine-3-carboxamide dihydrochloride

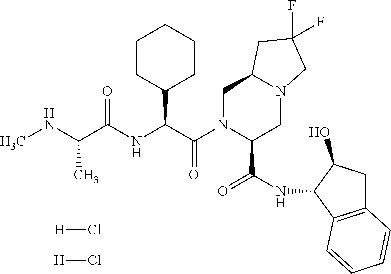

(1S,2S)-1-(1[(3S,8aR)-2-{(2S)-2-Cyclohexyl-2-[(N-methyl-L-alanyl)amino]acetyl}-7,7-difluorooctahydropyrrolo[1,2-a]pyrazin-3-yl]carbonyl)amino)-2,3-dihydro-1H-inden-2-yl acetate dihydrochloride (40 mg) was dissolved in methanol (1.6 mL), 1M potassium carbonate (0.400 mL) was added thereto, and the mixture was stirred at room temperature for 2 hr. Water was added to the reaction mixture, and the mixture was extracted twice with ethyl acetate/tetrahydrofuran. The extract was dried over anhydrous magnesium sulfate, and filtered. The filtrate was concentrated under reduced pressure, and the residue was purified by silica gel column chromatography (ethyl acetate/methanol=80/20-50/50). The object fractions were collected, the solvent was evaporated under reduced pressure, and the residue was dried in vacuum. To the obtained residue was added 1M hydrogen chloride-diethyl ether solution (2.0 mL), and the mixture was stirred at room temperature for 15 min, and concentrated under reduced pressure. To the residue was added heptane/diethyl ether (=2/1), and the precipitate was collected by filtration, and washed with heptane/diethyl ether (=3/1), and dried in vacuum for 30 min to give the title compound (10.5 mg) as a white powder.

LC-MS: 562.4 (MH⁺).

¹H NMR (DMSO-d₆, 300 MHz): δ 0.90-1.36 (9H, N), 1.53-2.09 (7H, m), 2.15-2.85 (8H, m), 3.04-3.73 (5H, m), 4.06-5.34 (6H, m), 7.03-7.22 (4H, m), 8.12-8.23 (1H, m), 8.36-8.60 (1H, m).

### Example 53

### Production of (3S,8aR)-2-{(2S)-2-cyclohexyl-2-[(N-methyl-L-alanyl)amino]acetyl}-N-[(1S,2S)-2-fluoro-2,3-dihydro-1H-inden-1-yl]octahydropyrrolo[1,2-a]pyrazine-3-carboxamide dihydrochloride

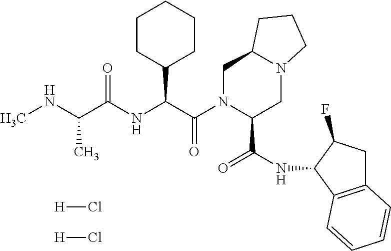

### (i) Production of tert-butyl(3S,8aR)-3-{[(1S,2R)-2-hydroxy-2,3-dihydro-1H-inden-1-yl]carbamoyl}hexahydropyrrolo[1,2-a]pyrazine-2(1H)-carboxylate

To a solution of 2′-tert-butyl 3-methyl(3S,8aR)-hexahydropyrrolo[1,2-a]pyrazine-2,3(1H)-dicarboxylate (2.0 g) in tetrahydrofuran (60 mL)/water (14 mL) was added lithium hydroxide monohydrate (472 mg) at room temperature, and the reaction mixture was stirred at 50° C. for 5 hr. The reaction mixture was allowed to cool to room temperature, neutralized with 3M hydrochloric acid (3.75 mL), and concentrated under reduced pressure. The residue was subjected twice to azeotropic distillation with toluene, and dried in vacuum for 1 hr. The obtained residue was dissolved in N,N-dimethylformamide (20 mL), N,N-diisopropylethylamine (2.45 mL), (1S,2R)-1-amino-2,3-dihydro-1H-inden-2-ol (1.36 g), 1-hydroxybenzotriazole (1.05 g) and 1-ethyl-3-(3-dimethylaminopropyl)carbodiimide hydrochloride (8.09 g) were added thereto, and the mixture was stirred at room temperature for 14 hr. The reaction mixture was poured into ethyl acetate/water, and the organic layer was separated. The organic layer was washed twice with saturated aqueous sodium hydrogen carbonate, washed with saturated brine (100 mL), dried over anhydrous magnesium sulfate, and filtered. The filtrate was concentrated under reduced pressure, and the residue was washed with hexane/ethyl acetate (=2/1), and dried in vacuum for 1 hr to give the title compound (2.03 g) as a white powder.

LC-MS: 402.3 (MH⁺).

¹H NMR (DMSO-d₆, 300 MHz): δ 1.14-1.47 (10H, m), 1.55-1.87 (4H, m), 1.89-2.03 (1H, m), 2.10-2.23 (1H, m), 2.75-3.12 (4H, m), 3.40-3.57 (1H, m), 3.87-3.98 (1H, m), 4.35-4.47 (1H, m), 4.60-4.72 (1H, m), 5.07-5.24 (2H, m), 7.10-7.27 (4H, m), 7.56-7.80 (1H, m).

### (ii) Production of tert-butyl(3S,8aR)-3-{[(1S,2S)-2-fluoro-2,3-dihydro-1H-inden-1-yl]carbamoyl}hexahydropyrrolo[1,2-a]pyrazine-2(1H)-carboxylate

tert-Butyl(3S,8aR)-3-{[(1S,2R)-2-hydroxy-2,3-dihydro-1H-inden-1-yl]carbamoyl}hexahydropyrrolo[1,2-a]pyrazine-2(1H)-carboxylate (2.0 g) was dissolved in toluene (30 mL), and bis(2-methoxyethyl)aminosulfur trifluoride (1.75 mL) was added dropwise thereto under ice-cooling. The mixture was stirred for 30 min under ice-cooling, and then at room temperature for 4 hr. The reaction mixture was poured into ice-cooled 3% aqueous sodium bicarbonate, and the mixture was extracted with ethyl acetate. The extract was washed with saturated brine, dried over anhydrous magnesium sulfate, and filtered. The filtrate was concentrated under reduced pressure, and the residue was purified by silica gel column chromatography (hexane/ethyl acetate=75/25→0/100). The object fractions were collected, and the solvent was evaporated under reduced pressure to give the title compound (730 mg) as a colorless oil.

LC-MS: 404.3 (MH⁺).

¹H NMR (DMSO-d₆, 300 MHz): δ 1.17-1.45 (10H, m), 1.55-1.85 (4H, m), 1.86-2.04 (1H, m), 2.12-2.27 (1H, m), 2.76-3.14 (3H, m), 3.28-3.49 (2H, m), 3.85-3.98 (1H, m), 4.38-4.56 (1H, m), 5.07-5.42 (2H, m), 7.14-7.30 (4H, m), 8.28-8.49 (1H, m).

### (iii) Production of tert-butyl{(1S)-1-cyclohexyl-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxoethyl}carbamate

tert-Butyl(3S,8aR)-3-{[(1S,2S)-2-fluoro-2,3-dihydro-1H-inden-1-yl]carbamoyl}hexahydropyrrolo[1,2-a]pyrazine-2(1H)-carboxylate (400 mg) was dissolved in ethyl acetate (2.0 mL), 4M hydrogen chloride-ethyl acetate solution (8.0 mL) was added thereto, and the mixture was stirred at room temperature for 1 hr, and concentrated under reduced pressure. To the residue was added ethyl acetate, and the mixture was stirred at room temperature for 30 min. The precipitate was collected by filtration, washed with ethyl acetate, and dried in vacuum. To the obtained residue was added N,N-dimethylformamide (8.0 mL),
N,N-diisopropylethylamine (1.04 mL), (2S)-[(tert-butoxycarbonyl)amino](cyclohexyl)ethanoic acid (332 mg), 1-hydroxybenzotriazole (188 mg) and 1-ethyl-3-(3-dimethylaminopropyl)carbodiimide hydrochloride (380 mg) were added thereto, and the mixture was stirred at room temperature for 3 hr. Water was added to the reaction mixture, and the mixture was extracted with ethyl acetate. The extract was washed twice with saturated aqueous sodium hydrogen carbonate, washed with saturated brine, dried over anhydrous magnesium sulfate, and filtered. The filtrate was concentrated under reduced pressure, and the residue was purified by silica gel column chromatography (hexane/ethyl acetate=40/60→0/100). The object fractions were collected, the solvent was evaporated under reduced pressure, and the residue was dried in vacuum for 1 hr to give the title compound (318 mg) as a white powder.

¹H NMR (DMSO-d₆, 300 MHz): δ 0.81-1.41 (15H, m), 1.45-1.87 (10H, m), 1.94-2.20 (1H, m), 2.45-2.57 (1H, m), 2.90-3.25 (3H, m), 3.26-3.74 (2H, m), 3.99-5.46 (5H, m), 6.86 (1H, d, J=8.4 Hz), 7.13-7.29 (4H, m), 8.16-8.29 (1H, m).

### (iv) Production of tert-butyl[(1S)-2-({(1S)-1-cyclohexyl-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxoethyl}amino)-1-methyl-2-oxoethyl]methylcarbamate

tert-Butyl{(1S)-1-cyclohexyl-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxoethyl}carbamate (400 mg) was dissolved in ethyl acetate (2.0 mL), 4M hydrogen chloride-ethyl acetate solution (8.0 mL) was added thereto, and the mixture was stirred at room temperature for 30 min, and concentrated under reduced pressure. To the residue was added ethyl acetate, and the mixture was stirred at room temperature for 1 hr. The precipitate was collected by filtration, washed with ethyl acetate, and dried in vacuum. The obtained residue was dissolved in N,N-dimethylformamide (8.0 mL), N,N-diisopropylethylamine (0.77 mL), N-(tert-butoxycarbonyl)-N-methyl-L-alanine (0.20 g), 1-hydroxybenzotriazole (0.14 g) and 1-ethyl-3-(3-dimethylaminopropyl)carbodiimide hydrochloride (0.25 g) were added thereto, and the mixture was stirred at room temperature for 3 hr. Water was added to the reaction mixture, and the mixture was extracted with ethyl acetate. The extract was washed twice with saturated aqueous sodium hydrogen carbonate, washed with saturated brine, dried over anhydrous magnesium sulfate, and filtered. The filtrate was concentrated under reduced pressure, and the residue was purified by silica gel column chromatography (hexane/ethyl acetate=40/60→0/100). The object fractions were collected, the solvent was evaporated under reduced pressure, and the residue was dried in vacuum for 1 hr to give the title compound (318 mg) as a white powder.

LC-MS: 628.4 (MH⁺).

¹H NMR (DMSO-d₆, 300 MHz): δ 0.80-1.48 (18H, m), 1.48-2.20 (12H, m), 2.62-2.77 (3H, m), 2.92-3.24 (3H, m), 3.26-3.74 (2H, m), 4.35-5.46 (6H, m), 7.13-7.32 (4H, m), 7.60-7.95 (1H, m), 8.24-8.40 (1H, m).

### (v) Production of (3S,8aR)-2-{(2S)-2-cyclohexyl-2-[(N-methyl-L-alanyl)amino]acetyl}-N-[(1S,2S)-2-fluoro-2,3-dihydro-1H-inden-1-yl]octahydropyrrolo[1,2-a]pyrazine-3-carboxamide dihydrochloride

tert-Butyl[(1S)-2-({(1S)-1-cyclohexyl-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxoethyl}amino)-1-methyl-2-oxoethyl]methylcarbamate (315 mg) was dissolved in ethyl acetate (1.58 mL), 4M hydrogen chloride-ethyl acetate solution (9.45 mL) was added thereto, and the mixture was stirred at room temperature for 1 hr, and concentrated under reduced pressure. To the residue was added ethyl acetate, and the mixture was stirred at room temperature for 1 hr. The precipitate was collected by filtration, washed with ethyl acetate, and dried in vacuum to give the title compound (250 mg) as a white powder.

¹H NMR (DMSO-d₆, 300 MHz): δ 0.90-1.42 (8H, m), 1.45-2.20 (10H, m), 2.44-2.53 (3H, m), 2.93-3.97 (9H, m), 4.38-5.60 (5H, m), 7.21-7.38 (4H, m), 8.65-9.50 (4H, m), 11.90-12.50 (1H, m).

### Example 54

### Production of (3S,8aR)—N-[(1S,2S)-2-fluoro-2,3-dihydro-1H-inden-1-yl]-2-[(2S)-2-[(N-methyl-L-alanyl)amino]-2-(tetrahydro-2H-pyran-4-yl)acetyl]octahydropyrrolo[1,2-a]pyrazine-3-carboxamide dihydrochloride

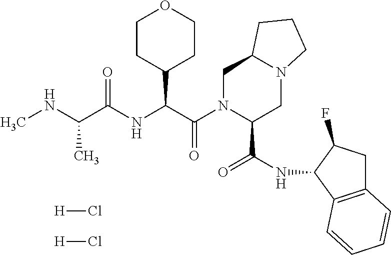

### (i) Production of benzyl[(1S)-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxo-1-(tetrahydro-2H-pyran-4-yl)ethyl]carbamate

To tert-butyl(3S,8aR)-3-{[(1S,2S)-2-fluoro-2,3-dihydro-1H-inden-1-yl]carbamoyl}hexahydropyrrolo[1,2-a]pyrazine-2(1H)-carboxylate (300 mg) were added ethyl acetate (1.5 mL) and 4M hydrogen chloride-ethyl acetate solution (6.0 mL), and the mixture was stirred at room temperature for 1 hr, and concentrated under reduced pressure. To the residue was added ethyl acetate, and the precipitate was collected by filtration, washed with ethyl acetate, and dried in vacuum for 30 min. The obtained residue was dissolved in N,N-dimethylformamide (6.0 mL), N,N-diisopropylethylamine (0.78 mL), (2S)-{[(benzyloxy)carbonyl]amino}(tetrahydro-2H-pyran-4-yl)ethanoic acid (283 mg), 1-hydroxybenzotriazole (141 mg) and 1-ethyl-3-(3-dimethylaminopropyl)carbodiimide hydrochloride (285 mg) were successively added thereto, and the mixture was stirred at room temperature for 3 hr. Water was added to the reaction mixture, and the mixture was extracted with ethyl acetate. The extract was washed with saturated aqueous sodium hydrogen carbonate and saturated brine, dried over anhydrous magnesium sulfate, and filtered. The filtrate was concentrated under reduced pressure, and the residue was purified by silica gel column chromatography (hexane/ethyl acetate=50/50→0/100). The object fractions were collected, and concentrated under reduced pressure, and the residue was dried in vacuum for 1 hr to give the title compound (249 mg) as a white powder.

¹H NMR (DMSO-d₆, 300 MHz): δ 1.15-1.42 (4H, m), 1.48-2.21 (8H, m), 2.90-3.90 (9H, m), 4.09-4.57 (2H, m), 4.80-5.41 (5H, m), 7.11-7.40 (9H, m), 7.46-7.63 (1H, m), 8.12-8.31 (1H, m).

### (ii) Production of tert-butyl[(1S)-2-{[(1S)-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxo-1-(tetrahydro-2H-pyran-4-yl)ethyl]amino}-1-methyl-2-oxoethyl]methylcarbamate

Benzyl[(1S)-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxo-1-(tetrahydro-2H-pyran-4-yl)ethyl]carbamate (240 mg) was dissolved in 5-10% hydrogen chloride-methanol solution (4.8 mL), 10% palladium-carbon (24 mg, 10 wt %) was added thereto, and the mixture was stirred at room temperature for 2 hr under a hydrogen atmosphere (3 atm). 10% Palladium-carbon (48 mg, 20 wt %) was added thereto, and the mixture was stirred again at room temperature for 2 hr under a hydrogen atmosphere (3 atm). After nitrogen substitution, the insoluble material was filtered off through celite, and washed with methanol. The filtrate was concentrated under reduced pressure to give an oil. To the obtained oil were added N,N-dimethylformamide (4.8 mL), N,N-diisopropylethylamine (433 μl), N-(tert-butoxycarbonyl)-N-methyl-L-alanine (110 mg), 1-hydroxybenzotriazole (78 mg) and 1-ethyl-3-(3-dimethylaminopropyl)carbodiimide hydrochloride (159 mg) were added thereto, and the mixture was stirred at room temperature for 2 hr. Water was added to the reaction mixture, and the mixture was extracted with ethyl acetate. The extract was washed twice with saturated aqueous sodium hydrogen carbonate, washed with saturated brine, dried over anhydrous magnesium sulfate, and filtered. The filtrate was concentrated under reduced pressure, and the residue was purified by silica gel column chromatography (ethyl acetate/methanol=100/0→92/8). The object fractions were collected, the solvent was evaporated under reduced pressure, and the residue was dried in vacuum for 1 hr to give the title compound (219 mg) as white crystals.

LC-MS: 630.4 (MH⁺).

¹H NMR (DMSO-d₆, 300 MHz): δ 1.10-1.45 (15H, m), 1.45-2.23 (9H, m), 2.62-2.78 (3H, m), 2.88-3.56 (10H, m), 4.35-5.45 (5H, m), 7.10-7.31 (4H, m), 7.70-8.10 (1H, m), 8.25-8.39 (1H, m).

### (iii) Production of (3S,8aR)—N-[(1S,2S)-2-fluoro-2,3-dihydro-1H-inden-1-yl]-2-[(2S)-2-[(N-methyl-L-alanyl)amino]-2-(tetrahydro-2H-pyran-4-yl)acetyl]octahydropyrrolo[1,2-a]pyrazine-3-carboxamide dihydrochloride

To tert-butyl[(1S)-2-{[(1S)-2-[(3S,8aR)-3-{[(1S,2S)-2-fluoro-2,3-dihydro-1H-inden-1-yl]carbamoyl}hexahydropyrrolo[1,2-a]pyrazin-2(1H)-yl]-2-oxo-1-(tetrahydro-2H-pyran-4-yl)ethyl]amino}-1-methyl-2-oxoethyl]methylcarbamate (216 mg) were added ethyl acetate (1.08 mL) and 4M hydrogen chloride-ethyl acetate solution (5.76 mL), and the mixture was stirred at room temperature for 1 hr, and concentrated under reduced pressure. To the residue was added ethyl acetate, and the precipitate was collected by filtration, washed with ethyl acetate, and dried in vacuum for 2 hr to give the title compound (180 mg) as a white powder.

LC-MS: 530.3 (MH⁺).

¹H NMR (DMSO-d₆, 300 MHz): δ 1.24-2.23 (14H, m), 2.90-3.98 (14H, m), 4.43-5.58 (5H, m), 7.18-7.42 (4H, m), 8.58-9.45 (4H, m), 11.80-12.50 (1H, m).

### Example 55

### Production of (3S,8aR)—N-[(4R)-3,4-dihydro-2H-chromen-4-yl]-7,7-difluoro-2-[(2S)-2-[(N-methyl-L-alanyl)amino]-2-(tetrahydro-2H-pyran-4-yl)acetyl]octahydropyrrolo[1,2-a]pyrazine-3-carboxamide

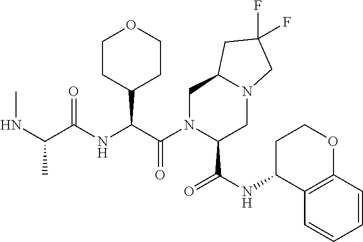

### (i) Production of tert-butyl(3S,8aR)-3-[(4R)-3,4-dihydro-2H-chromen-4-ylcarbamoyl]-7,7-difluorohexahydropyrrolo[1,2-a]pyrazine-2(1H)-carboxylate

To 2-tert-butyl 3-methyl(3S,8aR)-7,7-difluorohexahydropyrrolo[1,2-a]pyrazine-2,3(1H)-dicarboxylate (0.70 g) were added tetrahydrofuran (21 mL)/water (4.9 mL) and lithium hydroxide monohydrate (147 mg), and the mixture was stirred at 50° C. for 5 hr. The mixture was allowed to cool to room temperature, 1M hydrochloric acid (3.5 mL) was added thereto, and the mixture was concentrated under reduced pressure. Toluene was added thereto, the mixture was concentrated again under reduced pressure, and the residue was dried in vacuum. To the obtained residue were successively added N,N-dimethylformamide (14 mL), (4R)-3,4-dihydro-2H-chromen-4-amine hydrochloride (609 mg), N,N-diisopropylethylamine (761 μl), 1-hydroxybenzotriazole (354 mg) and 1-ethyl-3-(3-dimethylaminopropyl)carbodiimide hydrochloride (2.51 g), and the mixture was stirred at room temperature for 18 hr. To the reaction mixture were added ethyl acetate and water, and the mixture was extracted with ethyl acetate. The extract was washed with saturated aqueous sodium hydrogen carbonate and saturated brine, dried over anhydrous magnesium sulfate, and filtered. The filtrate was concentrated under reduced pressure, and the residue was purified by silica gel column chromatography (hexane/ethyl acetate=80/20→0/100). The object fractions were collected, the solvent was evaporated under reduced pressure, and the residue was dried in vacuum to give the title compound (450 mg).

¹H NMR (DMSO-d₆, 300 MHz): δ 1.36-1.44 (9H, m), 1.76-2.09 (4H, m), 2.21-2.57 (4H, m), 2.91-3.14 (1H, m), 3.28-3.48 (1H, m), 3.84-3.98 (1H, m), 4.15-4.26 (2H, m), 4.38-4.57 (1H, m), 4.97-5.13 (1H, m), 6.73-6.88 (2H, m), 7.09-7.19 (2H, m), 8.23-8.43 (1H, m).

### (ii) Production of benzyl[(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-(tetrahydro-2H-pyran-4-yl)ethyl]carbamate

tert-Butyl(3S,8aR)-3-[(4R)-3,4-dihydro-2H-chromen-4-ylcarbamoyl]-7,7-difluorohexahydropyrrolo[1,2-a]pyrazine-2(1H)-carboxylate (400 mg) was dissolved in 4M hydrogen chloride-ethyl acetate solution (4.0 mL), and the solution was stirred at room temperature for 30 min, and concentrated under reduced pressure. To the residue was added ethyl acetate (10 mL), and the mixture was stirred at room temperature for 20 min. The precipitate was collected by filtration, washed with ethyl acetate/diethyl ether (=1/1), and dried in vacuum for 30 min. The obtained powder was dissolved in N,N-dimethylformamide (8.0 mL), (2S)-{[(benzyloxy)carbonyl]amino}(tetrahydro-2H-pyran-4-yl)ethanoic acid (375 mg) and N,N-diisopropylethylamine (1.27 mL) were added thereto. Then, O-(7-azabenzotriazol-1-yl)-N,N,N′,N′-tetramethyluronium hexafluorophosphate (695 mg) was added thereto, and the mixture was stirred at room temperature for 16 hr. To the reaction mixture was added ethyl acetate/water, and the organic layer was separated. The organic layer was washed with saturated aqueous sodium hydrogen carbonate and saturated brine, dried over anhydrous magnesium sulfate, and filtered. The filtrate was concentrated under reduced pressure, and the residue was purified by basic silica gel column chromatography (hexane/ethyl acetate=70/30→0/100). The object fractions were collected, the solvent was evaporated under reduced pressure, and the residue was purified by silica gel column chromatography (hexane/ethyl acetate=70/30→0/100). The object fractions were collected, the solvent was evaporated under reduced pressure, and the residue was dried in vacuum for 1 hr to give the title compound (201 mg) as a white powder.

LC-MS: 613.3 (MH⁺).

¹H NMR (DMSO-d₆, 300 MHz): δ 1.20-1.69 (4H, m), 1.74-2.09 (4H, m), 2.13-2.73 (5H, m), 3.12-3.94 (6H, m), 4.08-4.57 (4H, m), 4.83-5.09 (4H, m), 6.70-6.88 (2H, m), 7.07-7.62 (8H, m), 8.11-8.27 (1H, m).

### (iii) Production of tert-butyl[(1S)-2-{[(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-(tetrahydro-2H-pyran-4-yl]ethyl}amino)-1-methyl-2-oxoethyl]methylcarbamate

To a solution of benzyl[(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-(tetrahydro-2H-pyran-4-yl)ethyl]carbamate (190 mg) in 5-10% hydrogen chloride-methanol (10 mL) was added 10% palladium-carbon (40 mg, 50% wet), and the mixture was stirred at room temperature for 2 hr under a hydrogen atmosphere (2 atm). The insoluble material was filtered off through a celite pad, and the filtrate was concentrated to give (3S,8aR)-2-[(2S)-2-amino-2-(tetrahydro-2H-pyran-4-yl)acetyl]-N-[(4R)-3,4-dihydro-2H-chromen-4-yl]-7,7-difluorooctahydropyrrolo[1,2-a]pyrazine-3-carboxamide dihydrochloride as a colorless oil. The obtained (3S,8aR)-2-[(2S)-2-amino-2-(tetrahydro-2H-pyran-4-yl)acetyl]-N-[(4R)-3,4-dihydro-2H-chromen-4-yl]-7,7-difluorooctahydropyrrolo[1,2-a]pyrazine-3-carboxamide dihydrochloride, N-(tert-butoxycarbonyl)-N-methyl-L-alanine (126 mg), O-(7-azabenzotriazol-1-yl)-N,N,N′,N′-tetramethyluronium hexafluorophosphate (235 mg) and N,N-diisopropylethylamine (0.325 mL) were mixed in N,N-dimethylformamide (3 mL), and the mixture was stirred at room temperature for 18 hr. The mixture was diluted with ethyl acetate (30 mL), and washed with saturated aqueous sodium hydrogen carbonate solution (50 mL) and saturated brine (50 mL). The organic layer was dried over anhydrous magnesium sulfate, and concentrated under reduced pressure. The residue was purified by silica gel column chromatography (ethyl acetate/hexane=10/90→100/0) to give the title compound (120 mg) as a colorless amorphous powder. LC-MS: 664 (MH⁺).

¹H NMR (DMSO-d₆, 300 MHz): δ 1.05-1.30 (5H, m), 1.39 (9H, s), 1.55 (2H, m), 1.77-2.09 (4H, m), 2.13-2.44 (3H, m), 2.53-2.70 (2H, m), 2.72-2.81 (2H, m), 3.10-3.29 (3H, m), 3.48 (2H, m), 3.57-4.12 (3H, m), 4.20 (2H, t, J=5.3 Hz), 4.41-4.81 (2H, m), 4.83-5.12 (2H, m), 6.70-6.90 (2H, m), 7.04-7.26 (2H, m), 7.87 (1H, brs), 8.19-8.36 (1H, m).

### (iv) Production of (3S,8aR)—N-[(4R)-3,4-dihydro-2H-chromen-4-yl]-7,7-difluoro-2-[(2S)-2-[(N-methyl-L-alanyl)amino]-2-(tetrahydro-2H-pyran-4-yl)acetyl]octahydropyrrolo[1,2-a]pyrazine-3-carboxamide

tert-Butyl[(1S)-2-{[(1S)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxo-1-(tetrahydro-2H-pyran-4-yl)ethyl]amino}-1-methyl-2-oxoethyl]methylcarbamate (120 mg) was dissolved in ethyl acetate (3 mL), 4M hydrogen chloride-ethyl acetate solution (2 mL) was added thereto, and the mixture was stirred at room temperature for 3 hr. The mixture was diluted with ethyl acetate (10 mL), and extracted with water (50 mL). The obtained aqueous layer was adjusted to pH 8 with 1M aqueous sodium hydroxide solution, and extracted with ethyl acetate (100 mL). The organic layer was dried over anhydrous magnesium sulfate, and concentrated under reduced pressure. The residue was recrystallized from a mixed solvent of ethyl acetate (5 mL) and hexane (2 mL), and collected by suction filtration to give the title compound (25 mg) as colorless crystals. In addition, the filtrate was concentrated under reduced pressure to give the title compound (70 mg) as an amorphous powder. LC-MS: 564 (MH⁺).

¹H NMR (DMSO-d₆, 300 MHz): δ 1.01-1.14 (3H, m), 1.16-1.63 (5H, m), 1.77-2.08 (5H, m), 2.17 (3H, s), 2.25-2.42 (3H, m), 2.71 (1H, m), 2.88-3.04 (1H, m), 3.17 (2H, m), 3.36-3.67 (2H, m), 3.84 (2H, m), 4.09-4.26 (3H, m), 4.81 (1H, m), 4.95-5.11 (2H, m), 6.71-6.90 (2H, m), 7.04-7.27 (2H, m), 8.04 (1H, m), 8.20-8.35 (1H, m).

### Example 56

### Production of (3S,8aR)—N-[(4R)-3,4-dihydro-2H-chromen-4-yl]-7,7-difluoro-2-[(2S)-2-[(N-methyl-L-alanyl)amino]-2-(tetrahydro-2H-pyran-4-yl)acetyl]octahydropyrrolo[1,2-a]pyrazine-3-carboxamide dihydrochloride

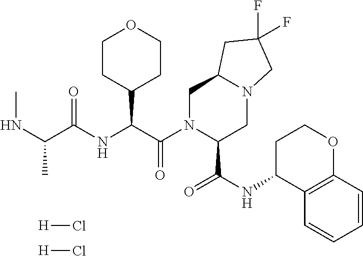

(3S,8aR)—N-[(4R)-3,4-Dihydro-2H-chromen-4-yl]-7,7-difluoro-2-[(2S)-2-[(N-methyl-L-alanyl)amino]-2-(tetrahydro-2H-pyran-4-yl)acetyl]octahydropyrrolo[1,2-a]pyrazine-3-carboxamide (70 mg) was dissolved in ethyl acetate (5 mL), 4M hydrogen chloride-ethyl acetate solution (2 mL) was added thereto, and the mixture was stirred at room temperature for 1 hr. The mixture was concentrated under reduced pressure, and the residue was collected by filtration, washed with ether, and dried under reduced pressure to give the title compound (60 mg) as a colorless amorphous powder. LC-MS: 564 (MH⁺).

¹H NMR (DMSO-d₆, 300 MHz): δ 1.35 (5H, d, J=7.0 Hz), 1.49 (3H, m), 1.77-2.15 (5H, m), 2.34 (4H, m), 2.71 (1H, m), 3.26 (3H, m), 3.35-3.69 (3H, m), 3.88 (3H, m), 4.06-5.11 (7H, m), 6.70-6.93 (2H, m), 7.03-7.27 (2H, m), 8.29 (1H, m), 8.59-8.83 (1H, m), 8.85-9.12 (1H, m).

### Example 57

### Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide dihydrochloride

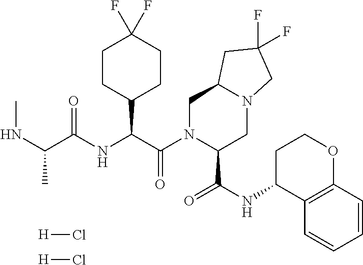

### (i) Production of benzyl{(1S)-1-(4,4-difluorocyclohexyl)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}carbamate

tert-Butyl(3S,8aR)-3-[(4R)-3,4-dihydro-2H-chromen-4-ylcarbamoyl]-7,7-difluorohexahydropyrrolo[1,2-a]pyrazine-2(1H)-carboxylate (1810 mg) was dissolved in ethyl acetate (5 mL), 4M hydrogen chloride-cyclopentyl methyl ether solution (5 mL) was added thereto, and the mixture was stirred at room temperature for 1 hr. The reaction solution was diluted with diethyl ether (5 mL), and the resulting solid was collected by suction filtration to give (3S,8aR)—N-[(4R)-3,4-dihydro-2H-chromen-4-yl]-7,7-difluorooctahydropyrrolo[1,2-a]pyrazine-3-carboxamide dihydrochloride as a colorless amorphous powder (1460 mg). The obtained (3S,8aR)—N-[(4R)-3,4-dihydro-2H-chromen-4-yl]-7,7-difluorooctahydropyrrolo[1,2-a]pyrazine-3-carboxamide dihydrochloride (1525 mg), (2S)-{[(benzyloxy)carbonyl]amino}(4,4-difluorocyclohexyl)ethanoic acid (1217 mg), O-(7-azabenzotriazol-1-yl)-N,N,N′,N′-tetramethyluronium hexafluorophosphate (2827 mg) and N,N-diisopropylethylamine (3.885 mL) were mixed in N,N-dimethylformamide (15 mL), and the mixture was stirred at room temperature for 18 hr. The mixture was diluted with ethyl acetate (200 mL), and washed with water (100 mL), saturated aqueous sodium hydrogen carbonate solution (100 mL) and saturated brine (100 mL). The organic layer was dried over anhydrous magnesium sulfate, and concentrated under reduced pressure. The residue was purified by silica gel column chromatography (ethyl acetate/hexane=20/80→60/40) to give the title compound (393 mg) as a white amorphous powder. LC-MS: 647 (MH⁺).

¹H NMR (DMSO-d₆, 300 MHz): δ 1.22-1.44 (2H, m), 1.55-2.08 (10H, m), 2.28 (3H, m), 2.63 (1H, s), 3.26 (1H, m), 3.37-3.70 (2H, m), 4.07-4.25 (3H, m), 4.48 (1H, m), 4.80-5.13 (4H, m), 6.68-6.91 (2H, m), 7.07-7.72 (8H, m), 8.09-8.29 (1H, m).

### (ii) Production of tert-butyl[(1S)-2-({(1S)-1-(4,4-difluorocyclohexyl)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}amino)-1-methyl-2-oxoethyl]methylcarbamate

To a solution of benzyl{(1S)-1-(4,4-difluorocyclohexyl)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}carbamate (2385 mg) in 5-10% hydrogen chloride-methanol (35 mL) was added 10% palladium-carbon (300 mg, 50% wet), and the mixture was stirred at room temperature for 2 hr under a hydrogen atmosphere (3 atm). The insoluble material was filtered off through a celite pad, and the filtrate was concentrated to give (3S,8aR)-2-[(2S)-2-amino-2-(4,4-difluorocyclohexyl)acetyl]-N-[(4R)-3,4-dihydro-2H-chromen-4-yl]-7,7-difluorooctahydropyrrolo[1,2-a]pyrazine-3-carboxamide dihydrochloride as a colorless oil. The obtained (3S,8aR)-2-[(2S)-2-amino-2-(4,4-difluorocyclohexyl)acetyl]-N-[(4R)-3,4-dihydro-2H-chromen-4-yl]-7,7-difluorooctahydropyrrolo[1,2-a]pyrazine-3-carboxamide dihydrochloride was dissolved in N,N-dimethylformamide (5 mL), a solution (10 mL) of N-(tert-butoxycarbonyl)-N-methyl-L-alanine (1041 mg), 1-ethyl-3-(3-dimethylaminopropyl)carbodimide hydrochloride (1965 mg), 1-hydroxybenzotriazole (554 mg) and N,N-diisopropylethylamine (1.785 mL) in N,N-dimethylformamide was added thereto, and the mixture was stirred at room temperature for 18 hr. The mixture was diluted with ethyl acetate (200 mL), and washed with water (100 mL), saturated aqueous sodium hydrogen carbonate solution (100 mL) and saturated brine (100 mL). The organic layer was dried over anhydrous magnesium sulfate, and concentrated under reduced pressure. The residue was recrystallized from a mixed solvent of ethyl acetate (15 mL) and hexane (5 mL), and collected by suction filtration to give the title compound (1400 mg) as colorless crystals. LC-MS: 698.4 (MH⁺).

¹H NMR (DMSO-d₆, 300 MHz): δ 1.20 (4H, m), 1.39 (9H, s), 1.54-2.07 (12H, m), 2.15-2.43 (3H, m), 2.54 (2H, m), 3.27 (1H, 1H), 3.35-3.71 (2H, m), 4.03 (2H, m), 4.20 (2H, m), 4.38-5.14 (4H, m), 6.67-6.96 (2H, m), 7.02-7.31 (2H, m), 7.91 (1H, m), 8.12-8.43 (1H, m).

### (iii) Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide dihydrochloride

tert-Butyl[(1S)-2-({(1S)-1-(4,4-difluorocyclohexyl)-2-[(3S,8aR)-3-[(4R)-3,4-dihydro-2H-chromen-4-ylcarbamoyl]-7,7-difluorohexahydropyrrolo[1,2-a]pyrazin-2(1H)-yl]-2-oxoethyl}amino)-1-methyl-2-oxoethyl]methylcarbamate (250 mg) was dissolved in methanol (2 mL), 4M hydrogen chloride-cyclopentyl methyl ether solution (5 mL) was added thereto, and the mixture was stirred at room temperature for 1 hr. The reaction solution was diluted with diethyl ether (5 mL), and the resulting solid was collected by suction filtration to give the title compound (150 mg) as a colorless amorphous powder. In addition, the filtrate was concentrated under reduced pressure to give the title compound (70 mg) as a colorless amorphous powder. LC-MS: 598.3 (MH⁺).

¹H NMR (DMSO-d₆, 300 MHz): δ 1.35 (5H, d, J=6.8 Hz), 1.42-2.15 (14H, m), 2.41-2.48 (4H, m), 3.15 (2H, s), 3.29-3.97 (5H, m), 4.64-5.12 (3H, m), 6.67-6.96 (2H, m), 7.04-7.34 (2H, m), 8.38 (1H, brs), 8.59-8.97 (2H, m), 9.29 (1H, brs).

### Example 58

### Production of (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide

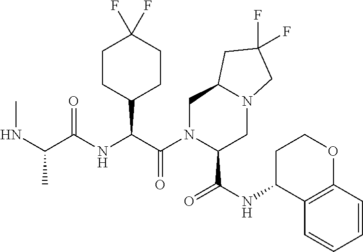

(3S,8aR)-2-{(2S)-2-(4,4-Difluorocyclohexyl)-2-[(N-methyl-L-alanyl)amino]acetyl}-N-[(4R)-3,4-dihydro-2H-chromen-4-yl]-7,7-difluorooctahydropyrrolo[1,2-a]pyrazine-3-carboxamide dihydrochloride (70 mg) was dissolved in ethyl acetate (5 mL), and the solution was washed with saturated aqueous sodium hydrogen carbonate solution (10 mL) and saturated brine (10 mL). The organic layer was dried over anhydrous magnesium sulfate, and concentrated under reduced pressure. The residue was recrystallized from a mixed solvent of ethyl acetate (5 mL) and hexane (3 mL), and collected by suction filtration to give the title compound (50 mg) as white crystals. LC-MS: 598.3 (MH⁺).

¹H NMR (DMSO-d₆, 300 MHz): δ 1.10 (3H, m), 1.25 (2H, m), 1.54-2.09 (12H, m), 2.17 (3H, m), 2.32 (4H, m), 2.98 (1H, m), 3.41-3.70 (2H, m), 4.02-4.72 (3H, m), 4.75-5.14 (3H, m), 6.78 (2H, m), 7.13 (2H, m), 7.92-8.39 (2H, m).

### Example 59

### Production of (1aR,4S,6aR,7aR)-5-{(2S)-2-cyclohexyl-2-[(N-methyl-L-alanyl)amino]acetyl}-N-[(4R)-3,4-dihydro-2H-chromen-4-yl]octahydro-1H-cyclopropa[4,5]pyrrolo[1,2-a]pyrazine-4-carboxamide dihydrochloride

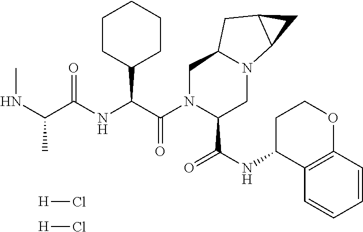

### (i) Production of 1-tert-butyl 2-ethyl(2R)-5-oxopyrrolidine-1,2-dicarboxylate

Ethyl 5-oxo-D-prolinate (25.0 g), 4-dimethylaminopyridine (1.94 mL) and di-tert-butyl bicarbonate (38.13 g) were mixed in acetonitrile (225 mL) under ice-cooling, and the mixture was allowed to warm to room temperature, and stirred for 18 hr. The mixture was concentrated under reduced pressure, and the residue was purified by silica gel column chromatography (ethyl acetate/hexane=9/91→80/20) to give the title compound (41.72 g) as a pale-yellow oil.

¹H NMR (CDCl₃, 300 MHz): δ 1.29 (3H, t, J=7.1 Hz), 1.50 (9H, s), 1.94-2.11 (1H, m), 2.21-2.42 (1H, m), 2.41-2.73 (2H, m), 4.24 (2H, q, J=7.1 Hz), 4.60 (1H, dd, J=9.4, 2.9 Hz).

### (ii) Production of 1-tert-butyl 2-ethyl(2R)-2,3-dihydro-1H-pyrrole-1,2-dicarboxylate

To a solution of 1-tert-butyl 2-ethyl(2R)-5-oxopyrrolidine-1,2-dicarboxylate (21.25 g) in toluene (165 mL) was added dropwise 1M lithium triethylborohydride tetrahydrofuran solution (90.85 mL) at −78° C., and the mixture was stirred at the same temperature for 1.5 hr. 4-Dimethylaminopyridine (201 mg) and diisopropylethylamine (64.05 g) were added thereto, trifluoroacetic anhydride (13.68 mL) was added slowly dropwise thereto at −78° C., and the mixture was allowed to warm to room temperature and stirred for 3 hr. The reaction mixture was diluted with ethyl acetate and water, the organic layer was dried over anhydrous magnesium sulfate, and the insoluble material was filtered off. The filtrate was concentrated under reduced pressure, and the residue was purified by silica gel column chromatography (ethyl acetate/hexane=0/100→66/34) to give the title compound (13.78 g) as a pale-yellow oil.

¹H NMR (CDCl₃, 300 MHz): δ 1.19-1.35 (3H, m), 1.40-1.54 (9H, m), 2.53-2.75 (1H, m), 2.95-3.18 (1H, m), 4.10-4.33 (2H, m), 4.50-4.70 (1H, m), 4.85-4.99 (1H, m), 6.46-6.71 (1H, m).

### (iii) Production of 2-tert-butyl 3-ethyl(1R,3R,5R)-2-azabicyclo[3.1.0]hexane-2,3-dicarboxylate

To a solution of 1-tert-butyl 2-ethyl(2R)-2,3-dihydro-1H-pyrrole-1,2-dicarboxylate (13.80 g) in toluene (400 mL) was added 1.1M diethylzinc toluene solution (114.4 mL) at −40° C., and the mixture was stirred at the same temperature for 30 min. Then, a solution (50 mL) of diiodomethane (61.28 g) in toluene was added thereto, and the mixture was stirred at −40 to −20° C. for 30 min, and then at 0° C. for 2 hr, and then at room temperature for 2 hr. To the reaction mixture were added aqueous saturated sodium hydrogen carbonate and ethyl acetate, and the insoluble material was filtered off through celite. The filtrate was partitioned, and the organic layer was dried over anhydrous magnesium sulfate, and concentrated under reduced pressure. The residue was purified by silica gel column chromatography (ethyl acetate/hexane=2/98→50/50) to give the title compound (5.69 g) as a colorless oil.

¹H NMR (CDCl₃, 300 MHz): δ 0.62-0.79 (1H, m), 0.85-0.94 (1H, m), 1.22-1.31 (3H, m), 1.39-1.56 (10H, m), 1.98-2.06 (1H, m), 2.48-2.68 (1H, m), 3.41-3.59 (1H, m), 4.09-4.25 (2H, m), 4.45-4.66 (1H, M).

### (iv) Production of tert-butyl(1R,3R,5R)-3-(hydroxymethyl)-2-azabicyclo[3.1.0]hexane-2-carboxylate.

To a solution of lithium aluminum hydride (1.68 g) in tetrahydrofuran (120 mL) was added a solution (20 mL) of 2-tert-butyl 3-ethyl(1R,3R,5R)-2-azabicyclo[3.1.0]hexane-2,3-dicarboxylate (5.55 g) in tetrahydrofuran under ice-cooling, and the mixture was stirred at 0° C. for 30 min. Sodium carbonate decahydrate (14.5 g) was added slowly under a nitrogen atmosphere, the mixture was stirred for 1 hr, and the insoluble material was filtered off through a celite pad. The filtrate was concentrated, and the residue was purified by silica gel column chromatography (ethyl acetate/hexane=2/98→66/34) to give the title compound (4.26 g) as a colorless oil.

¹H NMR (CDCl₃, 300 MHz): δ 0.40 (1H, brs), 0.73-0.85 (1H, m), 1.38-1.70 (3H, m), 1.50 (9H, s), 2.38-2.51 (1H, m), 3.39-3.54 (2H, m), 4.28-4.41 (1H, m), 4.77-4.89 (1H, m).

### (v) Production of tert-butyl(1R,3R,5R)-3-formyl-2-azabicyclo[3.1.0]hexane-2-carboxylate

To a solution of tert-butyl(1R,3R,5R)-3-(hydroxymethyl)-2-azabicyclo[3.1.0]hexane-2-carboxylate (4.27 g) and triethylamine (8.1 g) in a mixed solvent of dimethylsulfoxide (4 mL) and ethyl acetate (16 mL) was added slowly dropwise a solution (28 mL) of sulfur trioxide-pyridine (7.97 g) in dimethylsulfoxide under ice-cooling. The mixture was stirred at 0° C. for 30 min, and then at room temperature for 30 min. 1M Hydrochloric acid (80 mL) was added slowly dropwise thereto under ice-cooling, and the mixture was diluted with water and ethyl acetate. The organic layer was washed with water, dried over anhydrous magnesium sulfate, and concentrated under reduced pressure to give the title compound (300 mg) as a crude product. Without further purification, the compound was used for the next reaction.

¹H NMR (CDCl₃, 300 MHz): δ 0.26-0.39 (1H, m), 0.70-0.89 (1H, m), 1.32-1.77 (9H, m), 2.11-2.26 (1H, m), 2.30-2.57 (1H, m), 2.62 (1H, s), 3.46-3.67 (1H, m), 4.40-4.63 (1H, m), 9.32-9.53 (1H, m).

### (vi) Production of tert-butyl(1R,3R,5R)-3-[(benzylamino)methyl]-2-azabicyclo[3.1.0]hexane-2-carboxylate

To a solution of tert-butyl(1R,3R,5R)-3-formyl-2-azabicyclo[3.1.0]hexane-2-carboxylate and benzylamine (2.36 g) in tetrahydrofuran (100 mL) was added slowly dropwise sodium triacetoxyborohydride (6.36 g), and the mixture was stirred at room temperature for 1.5 hr. To the mixture were added water and ethyl acetate, and the mixture was partitioned. The organic layer was dried over anhydrous magnesium sulfate, and concentrated under reduced pressure. The residue was purified by silica gel column chromatography (ethyl acetate/hexane=9/91→100/0) to give the title compound (5.14 g) as a pale-red solid.

LC-MS: 303 (MH⁺).

¹H NMR (CDCl₃, 300 MHz): δ 0.28-0.59 (1H, m), 0.63-0.80 (1H, m), 1.30-1.60 (10H, m), 1.64-1.95 (1H, m), 2.33-2.53 (1H, m), 2.64-2.83 (1H, m), 3.32-4.42 (4H, m), 5.32 (2H, s), 7.19-7.41 (5H, m).

### (vii) Production of 1-[(1R,3R,5R)-2-azabicyclo[3.1.0]hexa-3-yl]-N-benzylmethanamine

To a solution of tert-butyl(1R,3R,5R)-3-[(benzylamino)methyl]-2-azabicyclo[3.1.0]hexane-2-carboxylate (5.40 g) in ethyl acetate (50 mL) was added 4M hydrogen chloride-ethyl acetate solution (50 mL), and the mixture was stirred at room temperature for 18 hr. The reaction mixture was concentrated under reduced pressure, the residue was dissolved in methanol, and the solution was filtered through a methanol suspension of Amberlyst A21. The filtered methanol solution was concentrated under reduced pressure to give the title compound as a pale-red oil.

LC-MS: 203 (MH⁺).

¹H NMR (DMSO-d₆, 300 MHz): δ 0.06-0.14 (1H, m), 0.52-0.62 (1H, m), 1.25-1.38 (2H, m), 2.01-2.37 (5H, m), 2.57 (1H, td, J=6.2, 2.6 Hz), 3.32 (2H, brs), 3.44-3.58 (1H, m), 7.13-7.35 (5H, m).

### (viii) Production of methyl(1aR,4S,6aR,7aR)-5-benzyloctahydro-1H-cyclopropa[4,5]pyrrolo[1,2-a]pyrazine-4-carboxylate

A solution (15 mL) of 1-[(1R,3R,5R)-2-azabicyclo[3.1.0]hexa-3-yl]-N-benzylmethanamine (434 mg), methyl 2,3-dibromopropionate (476 mg) and triethylamine (0.906 mL) in toluene was stirred at 90° C. for 18 hr. The reaction mixture was partitioned between ethyl acetate and water, and the organic layer was dried over anhydrous magnesium sulfate, and concentrated under reduced pressure. The residue was purified by silica gel column chromatography (ethyl acetate/hexane=2/98→66/34) to give the title compound (141 mg) as a colorless oil. LC-MS: 287 (MH⁺).

¹H NMR (CDCl₃, 300 MHz): δ 0.41-0.51 (1H, m), 0.55-0.67 (1H, m), 1.18-1.30 (1H, m), 1.30-1.42 (1H, m), 1.99-2.12 (1H, m), 2.36-2.53 (2H, m), 2.70 (1H, t, J=11.3 Hz), 2.98 (1H, dd, J=11.3, 4.1 Hz), 3.05-3.19 (1H, m), 3.34-3.40 (1H, m), 3.55 (1H, dd, J=11.3, 2.3 Hz), 3.70 (3H, s), 3.90 (2H, d, J=1.3 Hz), 7.18-7.35 (5H, m).

### (ix) Production of 5-tert-butyl 4-methyl(1aR,4S,6aR,7aR)-octahydro-5H-cyclopropa[4,5]pyrrolo[1,2-a]pyrazine-4,5-dicarboxylate

Methyl(1aR,4S,6aR,7aR)-5-benzyloctahydro-1H-cyclopropa[4,5]pyrrolo[1,2-a]pyrazine-4-carboxylate (810 mg) was dissolved in 5% hydrogen chloride-methanol solution (25 mL), 10% palladium-carbon (150 mg, 50% wet) was added thereto, and the mixture was stirred at room temperature for 2 hr under a hydrogen atmosphere (1 atm). To the reaction mixture was added again 10% palladium-carbon (260 mg, 50% wet), and the mixture was stirred at room temperature for 5 hr under a hydrogen atmosphere (1 atm). The insoluble material was filtered off through a celite pad, and the filtrate was concentrated. Tetrahydrofuran (50 mL), saturated aqueous sodium hydrogen carbonate (50 mL) and di-tert-butyl dicarbonate (618 mg) were added thereto, and the mixture was stirred at room temperature for 18 hr. The reaction mixture was diluted with water and ethyl acetate, the organic layer was dried over magnesium sulfate, and the residue was purified by silica gel column chromatography (ethyl acetate/hexane=2/98→66/34) to give the title compound (691 mg) as a colorless solid.

LC-MS: 297 (MH⁺).

¹H NMR (CDCl₃, 300 MHz): δ 0.38-0.52 (1H, m), 0.62-0.76 (1H, m), 1.14-1.33 (1H, m), 1.35-1.51 (10H, m), 2.04-2.25 (1H, m), 2.36-2.53 (1H, m), 2.55-3.16 (3H, m), 3.45-3.69 (1H, m), 3.69-3.94 (1H, m), 3.70-3.80 (3H, m), 4.45-4.75 (1H, m).

### (x) Production of tert-butyl(1aR,4S,6aR,7aR)-4-[(4R)-3,4-dihydro-2H-chromen-4-ylcarbamoyl]octahydro-5H-cyclopropa[4,5]pyrrolo[1,2-a]pyrazine-5-carboxylate

5-tert-Butyl 4-methyl(1aR,4S,6aR,7aR)-octahydro-5H-cyclopropa[4,5]pyrrolo[1,2-a]pyrazine-4,5-dicarboxylate (691 mg) was dissolved in tetrahydrofuran (5 mL), a solution prepared by dissolving lithium hydroxide monohydrate (202 mg) in water (1.5 mL) was added thereto, and the mixture was stirred at 50° C. for 5 hr. To the mixture were added 1M hydrochloric acid (4.81 mL) and concentrated under reduced pressure. To the residue was added toluene, and the mixture was concentrated under reduced pressure. The residue was dissolved in N,N-dimethylformamide (120 mL), (4R)-3,4-dihydro-2H-chromen-4-amine hydrochloride (650 mg), 1-hydroxybenzotriazole (315 mg) and diisopropylethylamine (0.812 mL) were added thereto, and the mixture was stirred for 10 min. 1-Ethyl-3-(3-dimethylaminopropyl)carbodiimide hydrochloride (2.95 g) was added slowly thereto, and the mixture was stirred at room temperature for 18 hr. The reaction mixture was diluted with water and ethyl acetate, and the organic layer was washed with saturated aqueous sodium hydrogen carbonate and saturated sodium chloride water, and dried over anhydrous magnesium sulfate. The insoluble material was filtered off, the filtrate was concentrated under reduced pressure, and the residue was purified by silica gel column chromatography (ethyl acetate/hexane=9/91→100/0) to give the title compound (172 mg) as a colorless oil.

LC-MS: 414 (MH⁺).

¹H NMR (CDCl₃, 300 MHz): δ 0.27-0.49 (1H, m), 0.68-0.78 (1H, m), 1.10-1.70 (2H, m), 1.44 (9H, s), 1.91-2.08 (1H, m), 2.13-2.29 (2H, m), 2.45-2.68 (2H, m), 2.76-2.88 (1H, m), 2.97-3.11 (1H, m), 3.45-4.21 (3H, m), 4.19-4.31 (1H, m), 4.49-4.74 (1H, m), 5.12-5.26 (1H, m), 6.43-6.76 (1H, m), 6.79-6.96 (2H, m), 7.13-7.25 (2H, m).

### (xi) Production of tert-butyl[(1S)-1-cyclohexyl-2-{(1aR,4S,6aR,7aR)-4-[(4R)-3,4-dihydro-2H-chromen-4-ylcarbamoyl]octahydro-5H-cyclopropa[4,5]pyrrolo[1,2-a]pyrazin-5-yl}-2-oxoethyl]carbamate

To a solution of tert-butyl(1aR,4S,6aR,7aR)-4-[(4R)-3,4-dihydro-2H-chromen-4-ylcarbamoyl]octahydro-5H-cyclopropa[4,5]pyrrolo[1,2-a]pyrazine-5-carboxylate (171.5 mg) in ethyl acetate (20 mL) was added 4M hydrogen chloride-ethyl acetate solution (30 mL), and the mixture was stirred at room temperature for 2 hr. The reaction mixture was concentrated under reduced pressure, the residue was dissolved in N,N-dimethylformamide (5 mL), and (2S)-[(tert-butoxycarbonyl)amino](cyclohexyl)ethanoic acid (139 mg) and diisopropylethylamine (270 mg) were added thereto. Then O-(7-azabenzotriazol-1-yl)-N,N,N′,N′-tetramethyluronium hexafluorophosphate (240 mg) was added thereto, and the mixture was stirred at room temperature for 48 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrate was concentrated under reduced pressure, and the residue was purified by silica gel column chromatography (ethyl acetate/hexane=75/25→100/0-ethyl acetate/methanol=100/0→366/34) to give the title compound (181.5 mg) as a colorless amorphous powder. LC-MS: 553 (MH⁺).

¹H NMR (DMSO-d₆, 300 MHz): δ 0.35-0.51 (1H, m), 0.47-0.65 (1H, m), 0.81-3.48 (21H, m), 1.20-1.44 (9H, m), 3.65-3.95 (1H, m), 4.07-4.33 (3H, m), 4.48-4.68 (1H, m), 4.90-5.06 (1H, m), 6.63-6.91 (3H, m), 7.05-7.33 (2H, m), 8.16-8.36 (1H, m).

### (xii) Production of tert-butyl[(1S)-2-{[(1S)-1-cyclohexyl-2-[(1aR,4S,6aR,7aR)-4-[(4R)-3,4-dihydro-2H-chromen-4-ylcarbamoyl]octahydro-5H-cyclopropa[4,5]pyrrolo[1,2-a]pyrazin-5-yl]-2-oxoethyl]amino}-1-methyl-2-oxoethyl]methylcarbamate

To a solution of tert-butyl(1aR,4S,6aR,7aR)-4-[(4R)-3,4-dihydro-2H-chromen-4-ylcarbamoyl]octahydro-5H-cyclopropa[4,5]pyrrolo[1,2-a]pyrazine-5-carboxylate (181.5 mg) to in ethyl acetate (10 mL) was added 4M hydrogen chloride-ethyl acetate solution (20 mL), and the mixture was stirred at room temperature for 1.5 hr. The reaction mixture was concentrated under reduced pressure, and the residue was washed with diisopropyl ether, and dissolved in N,N-dimethylformamide (5 mL). N-(tert-Butoxycarbonyl)-N-methyl-L-alanine (80 mg), diisopropylethylamine (286 mg) and O-(7-azabenzotriazol-1-yl)-N,N,N′,N′-tetramethyluronium hexafluorophosphate (187 mg) were added thereto, and the mixture was stirred at room temperature for 1.5 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrated was concentrated under reduced pressure, and residue was purified by silica gel column chromatography (ethyl acetate/hexane=75/25→100/0→ethyl acetate/methanol=100/0→50/50) to give the title compound (77.1 mg) as a colorless amorphous powder. LC-MS: 638 (MH⁺).

¹H NMR (CDCl₃, 300 MHz): δ 0.34-0.47 (1H, m), 0.66-0.87 (1H, m), 0.81-3.27 (36H, m), 3.47-5.23 (7H, m), 6.49-8.08 (6H, m).

### (xiii) Production of (1aR,4S,6aR,7aR)-5-{(2S)-2-cyclohexyl-2-[(N-methyl-L-alanyl)amino]acetyl}-N-[(4R)-3,4-dihydro-2H-chromen-4-yl]octahydro-1H-cyclopropa[4,5]pyrrolo[1,2-a]pyrazine-4-carboxamide dihydrochloride

tert-Butyl[(1S)-2-{[(1S)-1-cyclohexyl-2-{(1aR,4S,6aR,7aR)-4-[(4R)-3,4-dihydro-2H-chromen-4-ylcarbamoyl]octahydro-5H-cyclopropa[4,5]pyrrolo[1,2-a]pyrazin-5-yl}-2-oxoethyl]amino}-1-methyl-2-oxoethyl]methylcarbamate (77.1 mg) was dissolved in ethyl acetate (5 mL), 4M hydrogen chloride-ethyl acetate solution (10 mL) was added thereto, and the mixture was stirred at room temperature for 1.5 hr. The mixture was concentrated under reduced pressure, and the residue was collected by filtration, washed with ether, and dried under reduced pressure to give the title compound (56.4 mg) as a pale-yellow amorphous powder. LC-MS: 538 (MH⁺).

¹H NMR (DMSO-d₆, 300 MHz): δ 0.78-1.44 (14H, m), 1.48-2.12 (12H, m), 3.05-4.40 (7H, m), 4.45-4.65 (1H, m), 4.64-4.83 (1H, m), 4.87-5.09 (1H, m), 6.69-6.98 (2H, m), 7.03-7.27 (2H, m), 8.62-8.94 (3H, m), 8.98-9.31 (1H, m), 12.60-12.84 (1H, m).

### Example 60

### Production of (1aR,4S,6aR,7aR)-5-{(2S)-2-cyclohexyl-2-[(N-methyl-L-alanyl)amino]acetyl}-N-[(1S,2S)-2-hydroxy-2,3-dihydro-1H-inden-1-yl]octahydro-1H-cyclopropa[4,5]pyrrolo[1,2-a]pyrazine-4-carboxamide dihydrochloride

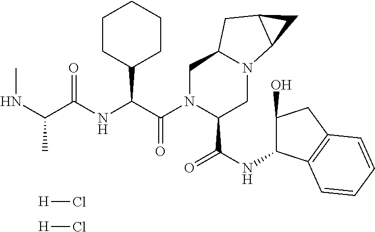

### (i) Production of tert-butyl(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazine-5-carboxylate

5-tert-Butyl 4-methyl(1aR,4S,6aR,7aR)-octahydro-5H-cyclopropa[4,5]pyrrolo[1,2-a]pyrazine-4,5-dicarboxylate (388 mg) was dissolved in tetrahydrofuran (5 mL), a solution prepared by dissolving lithium hydroxide monohydrate (131 mg) in water (1.5 mL) was added thereto, and the mixture was stirred at 50° C. for 8 hr. To the mixture were added 1M hydrochloric acid (3.12 mL) and concentrated under reduced pressure. To the residue was added toluene, and the mixture was concentrated under reduced pressure. The residue was dissolved in N,N-dimethylformamide (30 mL), (1S,2S)-1-amino-2,3-dihydro-1H-inden-2-ol (277 mg), 1-hydroxybenzotriazole (168 mg) and diisopropylethylamine (0.432 mL) were added thereto, and the mixture was stirred for 10 min. 1-Ethyl-3-(3-dimethylaminopropyl)carbodiimide hydrochloride (1.60 g) was added slowly thereto, and the mixture was stirred at room temperature for 18 hr. The reaction mixture was diluted with water and ethyl acetate, and the organic layer was washed with saturated aqueous sodium hydrogen carbonate and saturated sodium chloride water, and dried over anhydrous magnesium sulfate. The insoluble material was filtered off, the filtrate was concentrated under reduced pressure, and the residue was purified by silica gel column chromatography (ethyl acetate/hexane=9/91→100/0) to give the title compound (400 mg) as a colorless solid.

LC-MS: 414 (MH⁺).

¹H NMR (DMSO-d₆, 300 MHz): δ 0.36-0.47 (1H, m), 0.55-0.74 (1H, m), 1.12-1.29 (1H, m), 1.29-1.52 (10H, m), 2.09-2.24 (1H, m), 2.60-2.93 (3H, m), 2.93-3.55 (4H, m), 3.62-3.83 (1H, m), 4.17-4.44 (2H, m), 4.91-5.07 (1H, m), 5.18-5.28 (1H, m), 7.04-7.24 (4H, m), 8.10-8.25 (1H, m).

### (ii) Production of tert-butyl{(1S)-1-cyclohexyl-2-[(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazin-5-yl]-2-oxoethyl}carbamate

To tert-butyl(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazine-5-carboxylate (400 mg) was added 5% hydrogen chloride-methanol solution (100 mL), and the mixture was stirred at room temperature for 3 hr, and then at 50° C. for 2.5 hr. The reaction mixture was concentrated under reduced pressure, and the precipitated solid was collected by filtration, washed with diisopropyl ether, and dried under reduced pressure. The residue was dissolved in N,N-dimethylformamide (20 mL), (2S)-[(tert-butoxycarbonyl)amino](cyclohexyl)ethanoic acid (293 mg), diisopropylethylamine (565 mg) and O-(7-azabenzotriazol-1-yl)-N,N,N′,N′-tetramethyluronium hexafluorophosphate (500 mg) were added thereto, and the mixture was stirred at room temperature for 74 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrated was concentrated under reduced pressure, and the residue was purified by silica gel column chromatography (ethyl acetate/hexane=9/91→100/0→ethyl acetate/methanol=100/0→50/50) to give the title compound (290 mg) as a colorless amorphous powder.

LC-MS: 553 (MH⁺).

¹H NMR (DMSO-d₆, 300 MHz): δ 0.42-0.80 (2H, m), 0.81-1.82 (22H, m), 2.05-2.34 (1H, m), 2.57-3.37 (7H, m), 3.63-4.34 (3H, m), 4.50-4.68 (1H, m), 4.89-5.30 (2H, m), 6.65-6.92 (1H, m), 7.06-7.32 (4H, m), 8.09-8.23 (1H, m).

### (iii) Production of tert-butyl[(1S)-2-({(1S)-1-cyclohexyl-2-[(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazin-5-yl]-2-oxoethyl}amino)-1-methyl-2-oxoethyl]methylcarbamate

To tert-butyl{(1S)-1-cyclohexyl-2-[(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazin-5-yl]-2-oxoethyl}carbamate (290 mg) was added 5% hydrogen chloride-methanol solution (60 mL), and the mixture was stirred at 50° C. for 1.5 hr. The reaction mixture was concentrated under reduced pressure, the residue was dissolved in methanol, and the solution was filtered through a methanol suspension of Amberlyst A21. The filtered methanol solution was concentrated under reduced pressure, and the residue was dissolved in N,N-dimethylformamide (5 mL). N-(tert-Butoxycarbonyl)-N-methyl-L-alanine (140 mg), diisopropylethylamine (0.460 mL) and O-(7-azabenzotriazol-1-yl)-N,N,N′,N′-tetramethyluronium hexafluorophosphate (300 mg) were added thereto, and the mixture was stirred at room temperature for 18 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrated was concentrated under reduced pressure, and the residue was purified by silica gel column chromatography (ethyl acetate/hexane=25/75→100/0) to give the title compound (230 mg) as a colorless amorphous powder.

LC-MS: 638 (MH⁺).

¹H NMR (DMSO-d₆, 300 MHz): δ 0.37-0.75 (2H, m), 0.77-3.41 (36H, m), 3.71-4.31 (2H, m), 4.39-4.74 (3H, m), 4.91-5.05 (1H, m), 5.09-5.31 (1H, m), 7.04-7.32 (4H, m), 7.59-7.99 (1H, m), 8.08-8.26 (1H, m).

### (iv) Production of (1aR,4S,6aR,7aR)-5-{(2S)-2-cyclohexyl-2-[(N-methyl-L-alanyl)amino]acetyl}-N-[(1S,2S)-2-hydroxy-2,3-dihydro-1H-inden-1-yl]octahydro-1H-cyclopropa[4,5]pyrrolo[1,2-a]pyrazine-4-carboxamide dihydrochloride

A solution (40 mL) of tert-butyl[(1S)-2-({(1S)-1-cyclohexyl-2-[(1aR,4S,6aR,7aR)-4-{[(1S,2S)-2-hydroxy-2,3-dihydro-1H-inden-1-yl]carbamoyl}octahydro-5H-cyclopropa[4,5]pyrrolo[1,2-a]pyrazin-5-yl]-2-oxoethyl}amino)-1-methyl-2-oxoethyl]methylcarbamate (230 mg) in 5% hydrogen chloride-methanol was stirred at room temperature for 18 hr. The mixture was concentrated under reduced pressure, and the residue was collected by filtration, washed with diisopropyl ether, and dried under reduced pressure to give the title compound (191.5 mg) as a colorless amorphous powder. LC-MS: 538 (MH⁺).

¹H NMR (DMSO-d₆, 300 MHz): δ 0.79-1.98 (10H, m), 2.34-3.97 (22H, m), 4.00-4.44 (2H, n), 4.53-5.03 (2H, m), 7.05-7.28 (4H, m), 8.62-9.04 (3H, m), 9.13-9.41 (1H, m), 12.94 (1H, brs).

### Example 61

### Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxy-7-(trifluoromethyl)octahydropyrrolo[1,2-a]pyrazine-3-carboxamide dihydrochloride

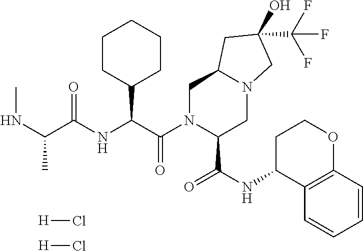

### (i) Production of 2-tert-butyl 3-methyl(3S,7R,8aR)-7-hydroxy-7-(trifluoromethyl)hexahydropyrrolo[1,2-a]pyrazine-2,3(1H)-dicarboxylate

To a solution of 2-tert-butyl 3-methyl(3S,8aR)-7-oxohexahydropyrrolo[1,2-a]pyrazine-2,3(1H)-dicarboxylate (1.0 g) in tetrahydrofuran (25 mL) were added trifluoromethyltrimethylsilane (953 mg) and 1M tetrabutylammonium fluoride tetrahydrofuran solution (7.04 mL) under ice-cooling, and the mixture was stirred at 0° C. for 30 min. The reaction mixture was diluted with ethyl acetate and water, and the organic layer was washed with saturated aqueous ammonium chloride and saturated brine, dried over magnesium sulfate, and concentrated under reduced pressure. The residue was purified by silica gel column chromatography (ethyl acetate/hexane=2/98→66/34) to give the title compound (1.05 g) as a colorless solid.

LC-MS: 369 (MH⁺).

¹H NMR (300 MHz, CDCl₃): δ 1.40-1.52 (9H, m), 1.50-1.67 (1H, m), 2.05-2.21 (1H, m), 2.28-2.56 (3H, m), 2.74-3.02 (1H, m), 3.08 (1H, d, J=10.4 Hz), 3.19 (1H, d, J=7.9 Hz), 3.47-3.57 (1H, m), 3.71-3.82 (3H, m), 3.92-4.18 (1H, m), 4.61-4.88 (1H, m).

### (ii) Production of tert-butyl(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazine-2(1H)-carboxylate

2-tert-Butyl 3-methyl(3S,7R,8aR)-7-hydroxy-7-(trifluoromethyl)hexahydropyrrolo[1,2-a]pyrazine-2,3(1H)-dicarboxylate (350 mg) was dissolved in tetrahydrofuran (5 mL), a solution prepared by dissolving lithium hydroxide monohydrate (86.0 mg) in water (1.5 mL) was added thereto, and the mixture was stirred at 55° C. for 3 hr. 1M Hydrochloric acid (2.05 mL) and toluene were added thereto, the mixture was concentrated under reduced pressure, and the residue was dissolved in N,N-dimethylformamide (10 mL). (4R)-3,4-Dihydro-2H-chromen-4-amine hydrochloride (265 mg), diisopropylethylamine (500 μL) and O-(7-azabenzotriazol-1-yl)-N,N,N′,N′-tetramethyluronium hexafluorophosphate (903 mg) were added thereto, and the mixture was stirred at room temperature for 18 hr. The reaction mixture was diluted with water and ethyl acetate, the organic layer was dried over anhydrous magnesium sulfate, and concentrated under reduced pressure, and the residue was purified by silica gel column chromatography (ethyl acetate/hexane=2/98→75/25) to give the title compound (324.3 mg) as a pale-yellow oil.

LC-MS: 486 (MH⁺).

¹H NMR (CDCl₃, 300 MHz): δ 1.37-1.47 (9H, m), 1.53-1.66 (1H, m), 1.70-2.08 (3H, m), 2.08-2.32 (2H, m), 2.37-2.60 (2H, m), 2.58-2.86 (1H, m), 3.11-3.22 (1H, m), 3.64-3.78 (1H, m), 4.01-4.32 (3H, m), 4.64-4.88 (1H, m), 5.19 (1H, brs), 6.50 (1H, brs), 6.80-6.87 (1H, m), 6.87-6.97 (1H, m), 7.18 (2H, t, J=7.20 Hz).

### (iii) Production of tert-butyl{(1S)-1-cyclohexyl-2-[(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazin-2(1H)-yl]-2-oxoethyl}carbamate

To a solution of tert-butyl(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazine-2(1H)-carboxylate (324.3 mg) in ethyl acetate (10 mL) was added 4M hydrogen chloride-ethyl acetate solution (120 mL), and the mixture was stirred at room temperature for 2 hr. The reaction mixture was concentrated under reduced pressure, and the precipitated solid was washed with diisopropyl ether and ethyl acetate, and dissolved in N,N-dimethylformamide (30 mL). (2S)-[(tert-Butoxycarbonyl)amino](cyclohexyl)ethanoic acid (223 mg), diisopropylethylamine (432 mg) and O-(7-azabenzotriazol-1-yl)-N,N,N′,N′-tetramethyluronium hexafluorophosphate (380 mg) were added thereto, and the mixture was stirred at room temperature for 2 hr. The reaction mixture was partitioned between water and ethyl acetate, and the organic layer was washed with water, and dried over magnesium sulfate. The insoluble material was filtered off, the filtrated was concentrated under reduced pressure, and the residue was purified by silica gel column chromatography (ethyl acetate/hexane=9/91→66/34) to give the title compound (236 mg) as a colorless solid.

LC-MS: 625 (MH⁺).

¹H NMR (CDCl₃, 300 MHz): δ 0.61-2.67 (29H, m), 2.91-3.34 (1H, m), 3.67-5.39 (7H, m), 5.94-8.05 (6H, m).

### (iv) Production of tert-butyl[(1S)-2-({(1S)-1-cyclohexyl-2-[(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazin-2(1H)-yl]-2-oxoethyl}amino)-1-methyl-2-oxoethyl]methylcarbamate

To a solution of tert-butyl[(1S)-1-cyclohexyl-2-[(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazin-2(1H)-yl]-2-oxoethyl]carbamate (250 mg) in ethyl acetate (5 mL) was added 4M hydrogen chloride-ethyl acetate solution (15 mL), and the mixture was stirred at room temperature for 3 hr. The reaction mixture was concentrated under reduced pressure, and the precipitated solid was washed with diisopropyl ether and ethyl acetate, and dissolved in N,N-dimethylformamide (3 mL). The solution was added to a solution of N-(tert-butoxycarbonyl)-N-methyl-L-alanine (50 mg), diisopropylethylamine (121 mg), 1-hydroxybenzotriazole (35 mg) and 1-ethyl-3-(3-dimethylaminopropyl)carbodiimide hydrochloride (54 mg) in N,N-dimethylformamide (5 mL), and the mixture was stirred at room temperature for 18 hr. The reaction mixture was partitioned between water and ethyl acetate, and the organic layer was washed with saturated aqueous sodium hydrogen carbonate and saturated brine, dried over magnesium sulfate, and concentrated under reduced pressure. The residue was purified by silica gel column chromatography (ethyl acetate/hexane=9/91→80/20) to give the title compound (99.3 mg) as a colorless amorphous powder.

LC-MS: 710 (MH⁺).

¹H NMR (DMSO-d₆, 300 MHz): δ 0.66-2.88 (36H, m), 2.95-3.29 (1H, m), 3.62-5.40 (7H, m), 6.22-8.02 (6H, m).

### (v) Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-hydroxy-7-(trifluoromethyl)octahydropyrrolo[1,2-a]pyrazine-3-carboxamide dihydrochloride

To a solution of tert-butyl[(1S)-2-({(1S)-1-cyclohexyl-2-[(3S,7R,8aR)-3-[(4R)-3,4-dihydro-2H-chromen-4-ylcarbamoyl]-7-hydroxy-7-(trifluoromethyl)hexahydropyrrolo[1,2-a]pyrazin-2(1H)-yl]-2-oxoethyl}amino)-1-methyl-2-oxoethyl]methylcarbamate (99.3 mg) in ethyl acetate (5 mL) was added 4M hydrogen chloride-ethyl acetate solution (10 mL), and the mixture was stirred at room temperature for 3 hr. The mixture was concentrated under reduced pressure, and the residue was collected by filtration, was washed with diisopropyl ether and ethyl acetate, and dried under reduced pressure to give the title compound (81.6 mg) as a colorless amorphous powder.

LC-MS: 610 (MH⁺).

¹H NMR (DMSO-d₆, 300 MHz): δ 0.76-4.33 (31H, m), 4.44-5.19 (4H, m), 6.31-7.40 (5H, m), 8.10-8.93 (3H, m), 9.00-9.25 (1H, m).

### Example 62

### Production of (1aS,4S,6aR,7aS)-5-{(2S)-2-cyclohexyl-2-[(N-methyl-L-alanyl)amino]acetyl}-N-[(4R)-3,4-dihydro-2H-chromen-4-yl]octahydro-1H-cyclopropa[4,5]pyrrolo[1,2-a]pyrazine-4-carboxamide dihydrochloride

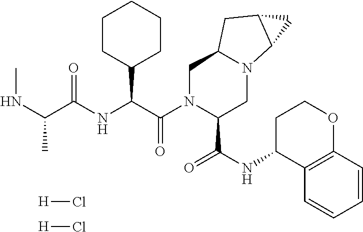

### (i) Production of 2-tert-butyl 3-ethyl(1S,3R,5S)-2-azabicyclo[3.1.0]hexane-2,3-dicarboxylate

To a solution of 1-tert-butyl 2-ethyl(2R)-2,3-dihydro-1H-pyrrole-1,2-dicarboxylate (13.80 g) in toluene (400 mL) was added 1.1M diethylzinc toluene solution (114.4 mL) at −40° C., and the mixture was stirred at −40° C. for 30 min. A solution (50 mL) of diiodomethane (61.28 g) in toluene was added thereto, and the mixture was stirred at −40 to −20° C. for 30 min, and then at 0° C. for 2 hr, and the at room temperature for 2 hr. To the reaction mixture were added saturated aqueous sodium hydrogen carbonate and ethyl acetate, and the insoluble material was filtered off through celite. The filtrate was partitioned, and the organic layer was dried over anhydrous magnesium sulfate, and concentrated under reduced pressure. The residue was purified by silica gel column chromatography (ethyl acetate/hexane=2/98→50/50) to give the title compound (1.09 g) as a colorless oil.

¹H NMR (CDCl₃, 300 MHz): δ 0.39-0.53 (1H, m), 0.72-0.88 (1H, m), 1.19-1.33 (3H, m), 1.37-1.65 (10H, m), 2.14-2.29 (1H, m), 2.28-2.42 (1H, m), 3.37-3.59 (1H, m), 3.91-4.12 (1H, m), 4.08-4.28 (2H, m).

### (ii) Production of tert-butyl(1S,3R,5S)-3-(hydroxymethyl)-2-azabicyclo[3.1.0]hexane-2-carboxylate

To a solution of lithium aluminum hydride (298 mg) in tetrahydrofuran (20 mL) was added a solution (20 mL) of 2-tert-butyl 3-ethyl(1S,3R,5S)-2-azabicyclo[3.1.0]hexane-2,3-dicarboxylate (1.0 g) in tetrahydrofuran under ice-cooling, and the mixture was stirred at 0° C. for 30 min. Sodium carbonate decahydrate (2.62 g) was added thereto under a nitrogen atmosphere, the mixture was stirred for 1 hr, and the insoluble material was filtered off through a celite pad. The filtrate was concentrated, and the residue was purified by silica gel column chromatography (ethyl acetate/hexane=2/98→66/34) to give the title compound (850 mg) as a colorless oil.

¹H NMR (CDCl₃, 300 MHz): δ 0.41 (1H, td, J=5.2, 2.4 Hz), 0.73 (1H, brs), 1.43-1.53 (1H, m), 1.49 (9H, s), 1.68-1.92 (1H, m), 2.15 (1H, dd, J=13.1, 8.2 Hz), 3.26 (1H, td, J=6.2, 2.4 Hz), 3.51-3.79 (3H, m), 4.86 (1H, brs).

### (iii) Production of tert-butyl(1S,3R,5S)-3-[(benzylamino)methyl]-2-azabicyclo[3.1.0]hexane-2-carboxylate

To a solution of tert-butyl(1S,3R,5S)-3-(hydroxymethyl)-2-azabicyclo[3.1.0]hexane-2-carboxylate (850 mg) and triethylamine (1.59 g) in a mixed solvent of dimethylsulfoxide (1.5 mL) and ethyl acetate (6 mL) was added dropwise a solution (10.5 mL) of sulfur trioxide-pyridine (1.60 g) in dimethylsulfoxide under ice-cooling, and the mixture was stirred at 0° C. for 30 min, and at room temperature for 1 hr. The reaction mixture was ice-cooled to 0° C., 1M hydrochloric acid (15.7 mL) was added dropwise, and the mixture was diluted with water and ethyl acetate. The organic layer was washed with water, dried over anhydrous magnesium sulfate, and concentrated under reduced pressure. The obtained residue and benzylamine (0.462 g) in tetrahydrofuran was added slowly sodium triacetoxyborohydride (1.25 g), and the mixture was stirred at room temperature for 18.5 hr. To the mixture were added water and ethyl acetate, the mixture was partitioned, and the organic layer was dried over anhydrous magnesium sulfate, and concentrated under reduced pressure. The residue was purified by silica gel column chromatography (ethyl acetate/hexane=25/75→100/0) to give the title compound (700 mg) as a pale-yellow solid.

LC-MS: 303 (MH⁺).

¹H NMR (CDCl₃, 300 MHz): δ 0.31-0.43 (1H, m), 0.75 (1H, brs), 1.30-1.57 (10H, m), 1.83-2.06 (1H, m), 2.07-2.23 (1H, m), 2.71-2.84 (1H, m), 2.93 (1H, brs), 3.08-3.31 (1H, m), 3.78-3.91 (2H, m), 3.92-4.12 (1H, m), 4.80 (1H, brs), 7.18-7.41 (5H, m).

### (iv) Production of 1-[(1S,3R,5S)-2-azabicyclo[3.1.0]hexa-3-yl]-N-benzylmethanamine dihydrochloride

To a solution of tert-butyl(1S,3R,5S)-3-[(benzylamino)methyl]-2-azabicyclo[3.1.0]hexane-2-carboxylate (700 mg) in ethyl acetate (10 mL) was added 4M hydrogen chloride-ethyl acetate solution (30 mL), and the mixture was stirred at room temperature for 3 hr. The reaction mixture was concentrated under reduced pressure, and the precipitated solid was collected by filtration to give the title compound (557 mg) as a colorless solid.

LC-MS: 203 (MH⁺).

### (v) Production of methyl(1aS,4S,6aR,7aS)-5-benzyloctahydro-1H-cyclopropa[4,5]pyrrolo[1,2-a]pyrazine-4-carboxylate

A solution (30 mL) of 1-[(1S,3R,5S)-2-azabicyclo[3.1.0]hexa-3-yl]-N-benzylmethanamine dihydrochloride (557 mg), methyl 2,3-dibromopropionate (512 mg) and triethylamine (4.62 mL) in toluene was stirred at 90° C. for 18 hr. The reaction mixture was partitioned between ethyl acetate and water, and the organic layer was dried over anhydrous magnesium sulfate, and concentrated under reduced pressure. The residue was purified by silica gel column chromatography (ethyl acetate/hexane=2/98-366/34) to give the title compound (45 mg) as a colorless oil.

¹H NMR (CDCl₃, 300 MHz): δ −0.09-0.07 (1H, m), 0.83-0.95 (1H, m), 1.18-1.37 (1H, m), 1.49-1.65 (1H, m), 1.68-1.83 (1H, m), 1.94-2.13 (1H, m), 2.55-2.75 (3H, m), 2.80-2.93 (1H, m), 3.34-3.50 (2H, m), 3.72 (3H, s), 3.80-4.00 (2H, m), 7.13-7.36 (5H, m).

### (vi) Production of 5-tert-butyl 4-methyl(1aS,4S,6aR,7aS)-octahydro-5H-cyclopropa[4,5]pyrrolo[1,2-a]pyrazine-4,5-dicarboxylate

To a solution of methyl(1aS,4S,6aR,7aS)-5-benzyloctahydro-1H-cyclopropa[4,5]pyrrolo[1,2-a]pyrazine-4-carboxylate (220 mg) in 5% hydrogen chloride-methanol (10 mL) was added 10% palladium-carbon (150 mg, 50% wet), and the mixture was stirred at room temperature for 3 hr under a hydrogen atmosphere (1 atm). The insoluble material was filtered off through a celite pad, and the filtrate was concentrated. To the residue were added tetrahydrofuran (10 mL), saturated aqueous sodium hydrogen carbonate (10 mL) and di-tert-butyl dicarbonate (168 mg), and the mixture was stirred at room temperature for 18 hr. The reaction mixture was diluted with water and ethyl acetate, the organic layer was dried over magnesium sulfate, and the residue was purified by silica gel column chromatography (ethyl acetate/hexane=2/98→66/34) to give the title compound (190 mg) as a colorless oil.

¹H NMR (CDCl₃, 300 MHz): δ 0.05 (1H, dt, J=8.1, 6.0 Hz), 0.77-0.90 (1H, m), 1.26-1.38 (1H, m), 1.38-1.50 (9H, m), 1.48-1.69 (1H, m), 1.75-1.95 (2H, m), 2.51 (1H, ddd, J=11.1, 4.3, 2.2 Hz), 2.60-2.90 (2H, m), 3.50-3.64 (1H, m), 3.69-3.81 (3H, m), 3.81-4.07 (1H, m), 4.54-4.82 (1H, m).

### (vii) Production of tert-butyl(1aS,4S,6aR,7aS)-4-[(4R)-3,4-dihydro-2H-chromen-4-ylcarbamoyl]octahydro-5H-cyclopropa[4,5]pyrrolo[1,2-a]pyrazine-5-carboxylate

5-tert-Butyl 4-methyl(1aS,4S,6aR,7aS)-octahydro-5H-cyclopropa[4,5]pyrrolo[1,2-a]pyrazine-4,5-dicarboxylate (691 mg) was dissolved in tetrahydrofuran (4 mL), a solution prepared by dissolving lithium hydroxide monohydrate (60.3 mg) in water (1.0 mL) was added thereto, and the mixture was stirred at 50° C. for 4 hr. To the reaction product was added toluene, and the mixture was concentrated under reduced pressure. The residue was dissolved in N,N-dimethylformamide (10 mL), (4R)-3,4-dihydro-2H-chromen-4-amine hydrochloride (200 mg) and diisopropylethylamine (280 mg) were added thereto, and the mixture was stirred for 5 min. O-(7-Azabenzotriazol-1-yl)-N,N,N′,N′-tetramethyluronium hexafluorophosphate (683 mg) was added thereto, the mixture was stirred at room temperature for 18 hr, and the reaction mixture was diluted with water and ethyl acetate. The organic layer was washed with saturated aqueous sodium hydrogen carbonate and saturated sodium chloride water, and dried over anhydrous magnesium sulfate. The insoluble material was filtered off, the filtrated was concentrated under reduced pressure, and the residue was purified by silica gel column chromatography (ethyl acetate/hexane=2/98→80/20) to give the title compound (254.4 mg) as a colorless amorphous solid.

LC-MS: 414 (MH⁺).

¹H NMR (CDCl₃, 300 MHz): δ 0.08 (1H, dt, J=8.1, 6.0 Hz), 0.78-0.86 (1H, m), 1.31-1.48 (10H, m), 1.49-1.63 (1H, m), 1.82-2.10 (2H, m), 2.14-2.33 (1H, m), 2.39-2.49 (1H, m), 2.50-2.84 (2H, m), 3.69-3.85 (1H, m), 3.93-4.32 (3H, m), 4.58-4.83 (1H, m), 5.14-5.26 (1H, m), 6.30-6.57 (1H, m), 6.78-6.95 (2H, m), 7.14-7.27 (3H, m).

### (viii) Production of tert-butyl[(1S)-1-cyclohexyl-2-[(1aS,4S,6aR,7aS)-4-[(4R)-3,4-dihydro-2H-chromen-4-ylcarbamoyl]octahydro-5H-cyclopropa[4,5]pyrrolo[1,2-a]pyrazin-5-yl]-2-oxoethyl]carbamate

To a solution (10 mL) of tert-butyl(1aS,4S,6aR,7aS)-4-[(4R)-3,4-dihydro-2H-chromen-4-ylcarbamoyl]octahydro-5H-cyclopropa[4,5]pyrrolo[1,2-a]pyrazine-5-carboxylate (254 mg) in methanol was added 4M hydrogen chloride-ethyl acetate solution (20 mL), and the mixture was stirred at room temperature for 3 hr. The reaction mixture was concentrated under reduced pressure, and the precipitated solid was washed with diisopropyl ether and ethyl acetate, and dissolved in N,N-dimethylformamide (10 mL). (2S)-[(tert-Butoxycarbonyl)amino](cyclohexyl)ethanoic acid (205 mg), diisopropylethylamine (397 mg) and O-(7-azabenzotriazol-1-yl)-N,N,N′,N′-tetramethyluronium hexafluorophosphate (397 mg) were added thereto, and the mixture was stirred at room temperature for 1 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrated was concentrated under reduced pressure, and the residue was purified by silica gel column chromatography (ethyl acetate/hexane=9/91→80/20) to give the title compound (250 mg) as a colorless oil. LC-MS: 553 (MH⁺).

¹H NMR (CDCl₃, 300 MHz): δ 0.03-0.16 (1H, m), 0.45-3.13 (31H, m), 3.68-5.40 (7H, m), 6.14-7.71 (5H, m).

### (ix) Production of tert-butyl[(1S)-2-{[(1S)-1-cyclohexyl-2-{(1aS,4S,6aR,7aS)-4-[(4R)-3,4-dihydro-2H-chromen-4-ylcarbamoyl]octahydro-5H-cyclopropa[4,5]pyrrolo[1,2-a]pyrazin-5-yl}-2-oxoethyl]amino}-1-methyl-2-oxoethyl]methylcarbamate

To a solution of tert-butyl(1aS,4S,6aR,7aS)-4-[(4R)-3,4-dihydro-2H-chromen-4-ylcarbamoyl]octahydro-5H-cyclopropa[4,5]pyrrolo[1,2-a]pyrazine-5-carboxylate (250 mg) in ethyl acetate (10 mL) was added 4M hydrogen chloride-ethyl acetate solution (20 mL), and the mixture was stirred at room temperature for 3.5 hr. The reaction mixture was concentrated under reduced pressure, and the precipitated solid was washed with diisopropyl ether and ethyl acetate, and dissolved in N,N-dimethylformamide (5 mL). N-(tert-Butoxycarbonyl)-N-methyl-L-alanine (120 mg), diisopropylethylamine (293 mg) and O-(7-azabenzotriazol-1-yl)-N,N,N′,N′-tetramethyluronium hexafluorophosphate (260 mg) were added thereto, and the mixture was stirred at room temperature for 3 hr. The reaction mixture was partitioned between water and ethyl acetate, and the organic layer was washed with saturated aqueous sodium hydrogen carbonate and saturated brine, and dried over magnesium sulfate. The insoluble material was filtered off, the filtrated was concentrated under reduced pressure and the residue was purified by silica gel column chromatography (ethyl acetate/hexane=9/91→80/20) to give the title compound (220 mg) as a colorless amorphous powder.

LC-MS: 638 (MH⁺).

¹H NMR (CDCl₃, 300 MHz): δ 0.03-0.19 (1H, m), 0.43-3.08 (37H, m), 3.73-5.31 (7H, m), 6.13-7.62 (6H, m).

### (x) Production of (1aS,4S,6aR,7aS)-5-{(2S)-2-cyclohexyl-2-[(N-methyl-L-alanyl)amino]acetyl}-N-[(4R)-3,4-dihydro-2H-chromen-4-yl]octahydro-1H-cyclopropa[4,5]pyrrolo[1,2-a]pyrazine-4-carboxamide dihydrochloride

To a solution of tert-butyl[(1S)-2-{[(1S)-1-cyclohexyl-2-{(1aS,4S,6aR,7aS)-4-[(4R)-3,4-dihydro-2H-chromen-4-ylcarbamoyl]octahydro-5H-cyclopropa[4,5]pyrrolo[1,2-a]pyrazin-5-yl}-2-oxoethyl]amino}-1-methyl-2-oxoethyl]methylcarbamate (220 mg) in ethyl acetate (10 mL) was added 4M hydrogen chloride-ethyl acetate solution (20 mL), and the mixture was stirred at room temperature for 3 hr. The mixture was concentrated under reduced pressure, and the residue was collected by filtration, washed with diisopropyl ether, and dried under reduced pressure to give the title compound (173 mg) as a colorless amorphous powder.

LC-MS: 538 (MH⁺).

¹H NMR (DMSO-d₆, 300 MHz): δ 0.76-2.57 (26H, m), 2.94-4.30 (6H, m), 4.37-4.60 (1H, m), 4.63-4.96 (2H, m), 4.91-5.09 (1H, m), 6.47-7.02 (2H, m), 7.05-7.65 (2H, m), 8.40-8.96 (3H, m), 9.04-9.37 (1H, m), 11.64-12.60 (1H, m).

### Example 63

### Production of (3S,8aR)-2-{(2S)-2-cyclohexyl-2-[(N-methyl-L-alanyl)amino]acetyl}-N-[(1R)-1-phenylethyl]octahydropyrrolo[1,2-a]pyrazine-3-carboxamide dihydrochloride

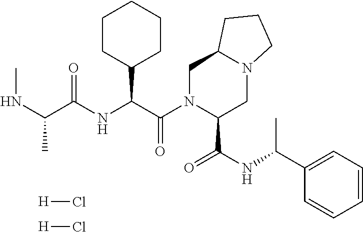

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) was added thereto, and the mixture was stirred at 50° C. for 3 hr. To the mixture were added (1R)-1-phenylethanamine (71 mg) and 4-(4,6-dimethoxy-1,3,5-triazin-2-yl)-4-methylmorpholinium chloride (232 mg), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=30/70→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 3 hr. To the mixture was added ethyl acetate (10 mL) and saturated aqueous sodium hydrogen carbonate solution (30 mL), and the organic layer was separated, washed with saturated brine (30 mL), dried over anhydrous magnesium sulfate, and concentrated under reduced pressure. The residue was dissolved in ethyl acetate (5 mL), 4M hydrogen chloride-ethyl acetate solution (0.100 mL) was added thereto, and the mixture was stirred at room temperature for 3 hr. The precipitated solid was collected by filtration, washed with ethyl acetate, and dried under reduced pressure to give the title compound (142 mg) as a colorless amorphous powder. LC-MS: 498.4 (MH⁺).

¹H NMR (DMSO-d₆, 300 MHz): δ 0.86-1.21 (5H, m), 1.30-1.43 (6H, m), 1.50-1.87 (8H, m), 1.97-2.18 (2H, m), 2.40-2.48 OH, m), 2.97-3.92 (7H, m) 4.38-4.52 (1H, m), 4.65-4.76 (2H, m), 4.79-4.93 (1H, m), 7.16-7.45 (5H, m), 8.49-9.08 (3H, m), 9.32-10.10 (1H, m), 12.01-12.52 (1H, m).

### Example 64

### Production of (3S,8aR)-2-{(2S)-2-cyclohexyl-2-[(N-methyl-L-alanyl)amino]acetyl}-N-(2-methylbenzyl)octahydropyrrolo[1,2-a]pyrazine-3-carboxamide dihydrochloride

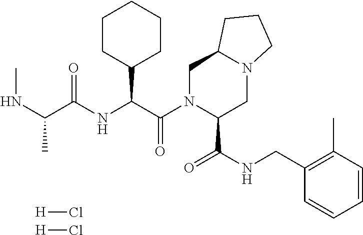

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) was added thereto, and the mixture was stirred at 50° C. for 3 hr. To the mixture were added 1-(2-methylphenyl)methanamine (0.073 mL) and 4-(4,6-dimethoxy-1,3,5-triazin-2-yl)-4-methylmorpholinium chloride (232 mg), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=30/70→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 3 hr. To the mixture were added ethyl acetate (10 mL) and saturated aqueous sodium hydrogen carbonate solution (30 mL), and the organic layer was separated, washed with saturated brine (30 mL), dried over anhydrous magnesium sulfate, and concentrated under reduced pressure. The residue was dissolved in ethyl acetate (5 mL), 4M hydrogen chloride-ethyl acetate solution (0.100 mL) was added thereto, and the mixture was stirred at room temperature for 3 hr. The precipitated solid was collected by filtration, washed with ethyl acetate, and dried under reduced pressure to give the title compound (98 mg) as a colorless amorphous powder. LC-MS: 498.4 (MH⁺).

¹H NMR (DMSO-d₆, 300 MHz): δ 0.89-1.25 (5H, m), 1.39 (3H, d, J=7.8 Hz), 1.52-1.86 (8H, m), 1.96-2.16 (2H, m), 2.25-2.48 (6H, m), 2.97-3.94 (7H, m) 4.23 (2H, d, J=5.5 Hz), 4.40-4.95 (3H, m), 7.08-7.29 (4H, m), 8.41-9.05 (3H, m), 9.40-10.30 (1H, m), 12.00-12.51 (1H, m).

### Example 65

### Production of (3S,8aR)-2-{(2S)-2-cyclohexyl-2-[(N-methyl-L-alanyl)amino]acetyl}-N-(1-methyl-1-phenylethyl)octahydropyrrolo[1,2-a]pyrazine-3-carboxamide dihydrochloride

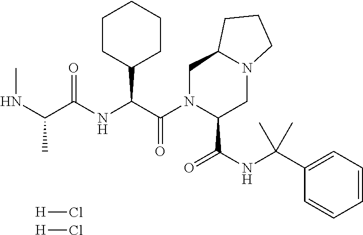

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) was added thereto, and the mixture was stirred at 50° C. for 3 hr. To the mixture were added 2-phenylpropan-2-amine (80 mg) and 4-(4,6-dimethoxy-1,3,5-triazin-2-yl)-4-methylmorpholinium chloride (232 mg), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=30/70→100/0) and then basic silica gel column chromatography (ethyl acetate/hexane=30/70→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 3 hr. To the mixture were added ethyl acetate (10 mL) and saturated aqueous sodium hydrogen carbonate solution (30 mL), and the organic layer was separated, washed with saturated brine (30 mL), dried over anhydrous magnesium sulfate, and concentrated under reduced pressure. The residue was dissolved in ethyl acetate (5 mL), 4M hydrogen chloride-ethyl acetate solution (0.100 mL) was added thereto, and the mixture was stirred at room temperature for 3 hr. The precipitated solid was collected by filtration, washed with ethyl acetate, and dried under reduced pressure to give the title compound (14 mg) as a colorless amorphous powder. LC-MS: 512.4 (MH⁺).

¹H NMR (DMSO-d₆, 300 MHz): δ 0.87-1.21 (5H, m), 1.37 (3H, d, J=7.8 Hz), 1.46-1.85 (14H, m), 1.97-2.14 (2H, m), 2.44-2.70 (3H, m), 2.98-3.95 (7H, m), 4.36-4.78 (3H, m), 7.10-7.39 (5H, m), 8.41-9.44 (4H, m), 11.73-12.45 (1H, m).

### Example 66

### Production of (3S,8aR)-2-{(2S)-2-cyclohexyl-2-[(N-methyl-L-alanyl)amino]acetyl}-N-(9H-fluoren-9-yl)octahydropyrrolo[1,2-a]pyrazine-3-carboxamide

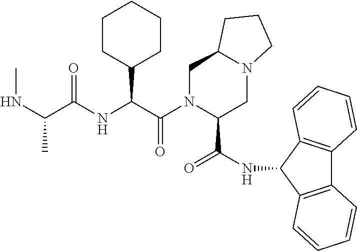

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) were added thereto, and the mixture was stirred at 50° C. for 3 hr. To the mixture was added 1M hydrochloric acid (0.59 mL), and the mixture was concentrated under reduced pressure to give a colorless amorphous powder. This amorphous powder, 9H-fluorene-9-amine hydrochloride (103 mg), 1-ethyl-3-(3-dimethylaminopropyl)carbodiimide hydrochloride (339 mg) and 1-hydroxybenzotriazole (79.7 mg) were mixed in N,N-dimethylformamide (3 mL), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=30/70→100/0) to give an amorphous powder. This amorphous powder was dissolved in ethyl acetate (4 mL), 4M hydrogen chloride-ethyl acetate solution (2 mL) was added thereto, and the mixture was stirred at room temperature for 3 hr. To the mixture were added ethyl acetate (10 mL) and saturated aqueous sodium hydrogen carbonate solution (30 mL), and the organic layer was separated, washed with saturated brine (30 mL), dried over anhydrous magnesium sulfate, and concentrated under reduced pressure. The residue was recrystallized from ethyl acetate-hexane to give the title compound (110 mg) as a white solid.

LC-MS: 558.4 (MH⁺).

¹H NMR (DMSO-d₆, 300 MHz): δ 0.87-1.27 (8H, m), 1.29-1.45 (1H, m), 1.52-1.89 (10H, m), 1.94-2.21 (6H, m), 2.79-3.04 (2H, m), 3.33-3.62 (2H, m), 4.08-4.17 (1H, m), 4.72-4.82 (1H, m), 5.06 (1H, d, J=3.2 Hz), 5.96-6.07 (1H, m), 7.26-7.64 (6H, m), 7.80-7.99 (3H, m), 8.33-8.55 (1H, m).

### Example 67

### Production of (3S,8aR)-2-{(2S)-2-cyclohexyl-2-[(N-methyl-L-alanyl)amino]acetyl}-N-[(4R)-6,7-difluoro-3,4-dihydro-2H-chromen-4-yl]octahydropyrrolo[1,2-a]pyrazine-3-carboxamide dihydrochloride

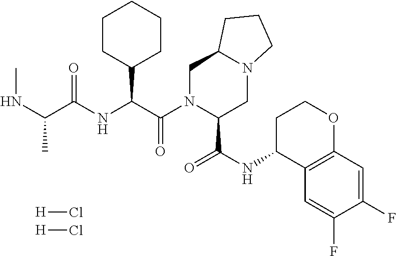

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (8.5 mL), a solution prepared by dissolving lithium hydroxide monohydrate (37.2 mg) in water (2.5 mL) was added thereto, and the mixture was stirred at 50° C. for 3 hr. To the mixture was added 1M hydrochloric acid (0.89 mL), and the mixture was concentrated under reduced pressure to give a colorless amorphous powder. This amorphous powder, (4R)-6,7-difluoro-3,4-dihydro-2H-chromen-4-amine hydrochloride (157 mg), 1-ethyl-3-(3-dimethylaminopropyl)carbodiimide hydrochloride (510 mg) and 1-hydroxybenzotriazole (120 mg) were mixed in N,N-dimethylformamide (3 mL), and the mixture was stirred at room temperature for 48 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 4 hr. The precipitated solid was collected by filtration, washed with ethyl acetate, and dried under reduced pressure to give the title compound (237 mg) as a colorless amorphous powder. LC-MS: 562.4 (MH⁺).

¹H NMR (DMSO-d₆, 300 MHz): δ 0.95-1.25 (5H, m), 1.37 (3H, d, J=6.8 Hz), 1.48-2.19 (12H, m), 2.40-2.48 (3H, m), 2.93-3.95 (7H, m), 4.15-4.32 (2H, m), 4.41-5.30 (4H, m), 6.80-6.98 (1H, m), 7.13-7.75 (1H, m), 8.54-10.79 (4H, m), 12.06-12.70 (1H, m).

### Example 6B

### Production of (3S,8aR)-2-{(2S)-2-cyclohexyl-2-[(N-methyl-L-alanyl)amino]acetyl}-N-[(4R)-7,8-difluoro-3,4-dihydro-2H-chromen-4-yl]octahydropyrrolo[1,2-a]pyrazine-3-carboxamide dihydrochloride

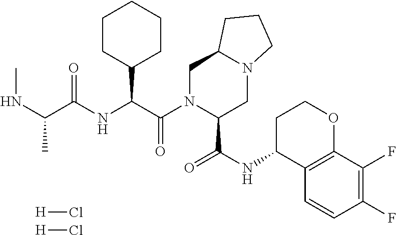

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) was added thereto, and the mixture was stirred at 50° C. for 3 hr. To the mixture was added 1M hydrochloric acid (0.59 mL), and the mixture was concentrated under reduced pressure to give a colorless amorphous powder. This amorphous powder, (4R)-7,8-difluoro-3,4-dihydro-2H-chromen-4-amine hydrochloride (157 mg), 1-ethyl-3-(3-dimethylaminopropyl)carbodiimide hydrochloride (226 mg) and 1-hydroxybenzotriazole (159 mg) were mixed in N,N-dimethylformamide (3 mL), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 4 hr. The precipitated solid was collected by filtration, washed with ethyl acetate, and dried under reduced pressure to give the title compound (119 mg) as a colorless amorphous powder. LC-MS: 562.4 (MH⁺).

¹H NMR (DMSO-d₆, 300 MHz): δ 0.92-1.27 (5H, m), 1.36 (3H, d, J=6.8 Hz), 1.48-2.17 (12H, m), 2.46 (3H, t, J=5.0 Hz), 2.89-3.95 (7H, m), 4.13-5.39 (6H, m), 6.76-7.46 (2H, m), 8.46-10.63 (4H, m), 12.03-12.61 (1H, m).

### Example 69

### Production of (3S,8aR)-2-{(2S)-2-cyclohexyl-2-[(N-methyl-L-alanyl)amino]acetyl}-N-[(1R)-4,4-difluoro-1,2,3,4-tetrahydronaphthalen-1-yl]octahydropyrrolo[1,2-a]pyrazine-3-carboxamide dihydrochloride

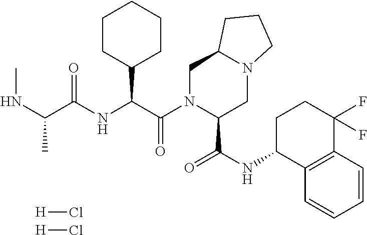

### (i) Production of (1R)-4,4-difluoro-1,2,3,4-tetrahydronaphthalen-1-amine

2,2,2-Trifluoro-N-[(1R)-4-oxo-1,2,3,4-tetrahydronaphthalen-1-yl]acetamide (1.71 g) and bis(2-methoxyethyl)aminosulfur trifluoride (2.21 g) were mixed at 0° C., and the mixture was stirred at 85° C. for 24 hr. The mixture was allowed to cool to room temperature, diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→50/50) to give a brown oil. This oil and 8M aqueous sodium hydroxide solution (1 mL) were mixed in ethanol (7 mL), and the mixture was stirred at room temperature for 2 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/methanol=100/0→90/10) to give the title compound (32.1 mg) as a brown oil.

¹H NMR (DMSO-d₆, 300 MHz): δ 1.64-1.80 (1H, m), 2.01-2.31 (2H, m), 2.39-2.59 (1H, m), 3.85-3.93 (1H, m), 7.33-7.40 (1H, m), 7.48 (1H, t, J=7.6 Hz), 7.55 (1H, d, J=7.7 Hz), 7.63 (1H, d, J=7.7 Hz)

### (ii) Production of (3S,8aR)-2-{(2S)-2-cyclohexyl-2-[(N-methyl-L-alanyl)amino]acetyl}-N-[(1R)-4,4-difluoro-1,2,3,4-tetrahydronaphthalen-1-yl]octahydropyrrolo[1,2-a]pyrazine-3-carboxamide dihydrochloride

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (5.7 mL), a solution prepared by dissolving lithium hydroxide monohydrate (24.8 mg) in water (1.5 mL) was added thereto, and the mixture was stirred at 50° C. for 3 hr. To the mixture was added 1M hydrochloric acid (0.59 mL), and the mixture was concentrated under reduced pressure to give a colorless amorphous powder. This amorphous powder, (1R)-4,4-difluoro-1,2,3,4-tetrahydronaphthalen-1-amine (20 mg), 1-ethyl-3-(3-dimethylaminopropyl)carbodiimide hydrochloride (226 mg) and 1-hydroxybenzotriazole (159 mg) were mixed in N,N-dimethylformamide (3 mL), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=50/50→100/0) and then basic silica gel column chromatography (ethyl acetate/hexane=30/70→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 3 hr. To the mixture were added ethyl acetate (10 mL) and saturated aqueous sodium hydrogen carbonate solution (30 mL), and the organic layer was separated, washed with saturated brine (30 mL), dried over anhydrous magnesium sulfate, and concentrated under reduced pressure. The residue was purified by silica gel column chromatography (ethyl acetate/methanol=100/0→90/10) to give a colorless amorphous powder. The residue was dissolved in ethyl acetate (5 mL), 4M hydrogen chloride-ethyl acetate solution (200 μL) was added thereto, and the mixture was stirred at room temperature for 3 hr. The precipitated-solid was collected by filtration, washed with ethyl acetate, and dried under reduced pressure to give the title compound (20.2 mg) as a colorless amorphous powder. LC-MS: 560.4 (MH⁺).

¹H NMR (DMSO-d₆, 300 MHz): δ 0.80-1.31 (6H, m), 1.32-1.43 (3H, m), 1.53-1.92 (8H, m), 1.94-2.17 (3H, m), 2.41-2.48 (3H, m), 2.96-4.55 (9H, m), 4.58-4.68 (1H, m), 4.71-4.84 (1H, m), 4.98-5.17 (1H, m), 7.04-7.79 (5H, m), 8.51-9.18 (3H, m), 9.22-9.54 (1H, m), 11.99-12.59 (1H, m).

### Example 70

### Production of (3S,8aR)-2-{(2S)-2-cyclohexyl-2-[(N-methyl-L-alanyl)amino]acetyl}-N-[(1R)-3,3-difluoro-2,3-dihydro-1H-inden-1-yl]octahydropyrrolo[1,2-a]pyrazine-3-carboxamide dihydrochloride

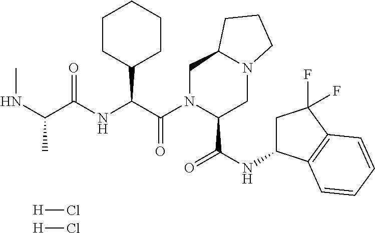

### (i) Production of (1R)-3,3-difluoro-2,3-dihydro-1H-inden-1-amine

2,2,2-Trifluoro-N-[(1R)-3-oxo-2,3-dihydro-1H-inden-1-yl]acetamide (3.50 g) and bis(2-methoxyethyl)aminosulfur trifluoride (5.31 g) were mixed at 0° C., and the mixture was stirred at 60° C. for 7 hr. The mixture was allowed to cool to room temperature, diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→30/70) to give a brown oil. This oil and 8M aqueous sodium hydroxide solution (0.5 mL) were mixed in ethanol (5 mL), and the mixture was stirred at room temperature for 3 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/methanol=100/0→90/10) to give the title compound (17.1 mg) as a brown oil.

¹H NMR (DMSO-d₆, 300 MHz): δ 2.05-2.42 (3H, m), 2.84-3.00 (1H, m), 4.34 (1H, q, J=6.4 Hz), 7.37-7.46 (1H, m), 7.49-7.61 (3H, m).

### (ii) Production of (3S,8aR)-2-{(2S)-2-cyclohexyl-2-[(N-methyl-L-alanyl)amino]acetyl}-N-[(1R)-3,3-difluoro-2,3-dihydro-1H-inden-1-yl]octahydropyrrolo[1,2-a]pyrazine-3-carboxamide dihydrochloride

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (67.6 mg) was dissolved in tetrahydrofuran (4 mL), a solution prepared by dissolving lithium hydroxide monohydrate (7.3 mg) in water (1 mL) was added thereto, and the mixture was stirred at 50° C. for 3 hr. To the mixture was added 1M hydrochloric acid (0.17 mL), and the mixture was concentrated under reduced pressure to give a colorless amorphous powder. This amorphous powder, (1R)-3,3-difluoro-2,3-dihydro-1H-inden-1-amine (15 mg), 1-ethyl-3-(3-dimethylaminopropyl)carbodiimide hydrochloride (127 mg) and 1-hydroxybenzotriazole (21.6 mg) were mixed in N,N-dimethylformamide (3 mL), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=30/70→100/0) to give a colorless amorphous powder. This amorphous powder was dissolved in ethyl acetate (4 mL), 4M hydrogen chloride-ethyl acetate solution (2 mL) was added thereto, and the mixture was stirred at room temperature for 3 hr. The precipitated solid was collected by filtration, washed with ethyl acetate, and dried under reduced pressure to give the title compound (20.3 mg) as a colorless amorphous powder. LC-MS: 546.3 (MH⁺).

¹H NMR (DMSO-d₆, 300 MHz): δ 0.92-1.41 (9H, m), 1.55-1.86 (6H, m), 1.94-2.21 (2H, m), 2.42-2.48 (3H, m), 2.92-3.13 (3H, m), 3.51-3.95 (6H, m), 4.42-4.67 (2H, m), 4.70-4.91 (1H, m), 5.28-5.47 (1H, m), 7.34-7.74 (5H, m), 8.73-9.13 (3H, m), 9.25-9.53 (1H, m), 12.02-12.48 (1H, m).

### Example 71

### Production of (3S,8aR)-2-{(2S)-2-cyclohexyl-2-[(N-methyl-L-alanyl)amino]acetyl}-N-[(1R)-2,3-dihydro-1H-inden-1-yl]octahydropyrrolo[1,2-a]pyrazine-3-carboxamide dihydrochloride

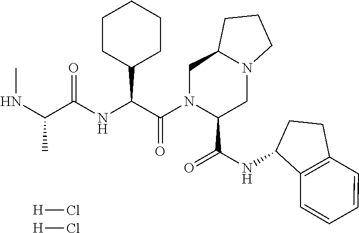

Methyl(3S,8aR)-2-[(2S-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (8.5 mL), a solution prepared by dissolving lithium hydroxide monohydrate (39 mg) in water (2.1 mL) was added thereto, and the mixture was stirred at 50° C. for 4 hr. To the mixture were added (1R)-2,3-dihydro-1H-inden-1-amine (79 mg) and 4-(4,6-dimethoxy-1,3,5-triazin-2-yl)-4-methylmorpholinium chloride (348 mg), and the mixture was stirred at room temperature for 19 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a colorless amorphous powder. This amorphous powder was dissolved in ethyl acetate (2 mL), 4M hydrogen chloride-ethyl acetate solution (8 mL) was added thereto, and the mixture was stirred at room temperature for 1 hr. The precipitated solid was collected by filtration, washed with ethyl acetate, and dried under reduced pressure to give the title compound (110 mg) as a colorless amorphous powder. LC-MS: 510.4 (MH⁺).

¹H NMR (DMSO-d₆, 300 MHz): δ 0.64-1.44 (11H, m), 1.44-2.24 (11H, m), 2.24-2.43 (2H, m), 2.56-3.24 (4H, m), 3.36-3.80 (3H, m), 3.88 (1H, brs), 4.50 (1H, brs), 4.61 (1H, brs), 4.76 (1H, brs), 5.24 (1H, s), 6.87-7.40 (4H, m), 8.17-8.94 (3H, m), 9.16 (1H, brs), 11.79 (1H, brs).

### Example 72

### Production of (3S,8aR)-2-{(2S)-2-cyclohexyl-2-[(N-methyl-L-alanyl)amino]acetyl}-N-[(4R)-7-fluoro-3,4-dihydro-2H-chromen-4-yl]octahydropyrrolo[1,2-a]pyrazine-3-carboxamide dihydrochloride

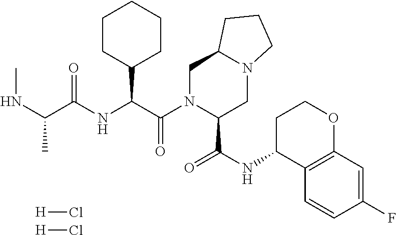

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (8.5 mL), a solution prepared by dissolving lithium hydroxide monohydrate (39 mg) in water (2.1 mL) was added thereto, and the mixture was stirred at 50° C. for 4 hr. To the mixture were added (4R)-7-fluoro-3,4-dihydro-2H-chromen-4-amine (120 mg) and 4-(4,6-dimethoxy-1,3,5-triazin-2-yl)-4-methylmorpholinium chloride (348 mg), and the mixture was stirred at room temperature for 19 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a colorless amorphous powder. This amorphous powder was dissolved in ethyl acetate (2 mL), 4M hydrogen chloride-ethyl acetate solution (8 mL) was added thereto, and the mixture was stirred at room temperature for 1 hr. The precipitated solid was collected by filtration, washed with ethyl acetate, and dried under reduced pressure to give the title compound (112 mg) as a colorless amorphous powder. LC-MS: 544.4 (MH⁺).

¹H NMR (DMSO-d₆, 300 MHz): δ 0.91-1.27 (6H, m), 1.36 (4H, d, J=6.8 Hz), 1.45-1.93 (10H, m), 1.99 (4H, s), 3.05 (1H, brs), 3.42-3.79 (5H, m), 3.86 (1H, brs), 4.23 (2H, brs), 4.47 (1H, brs), 4.59 (1H, brs), 4.76 (1H, brs), 4.96 (1H, brs), 6.55-6.77 (2H, m), 7.19 (1H, t, J=7.6 Hz), 8.87 (3H, m), 9.21 (1H, brs).

### Example 73

### Production of (3S,8aS)-2-[(2S)-2-[(N-methyl-L-alanyl)amino]-2-(tetrahydro-2H-pyran-4-yl)acetyl]-N-[(1R)-1,2,3,4-tetrahydronaphthalen-1-yl]octahydropyrrolo[1,2-a]pyrazine-3-carboxamide dihydrochloride

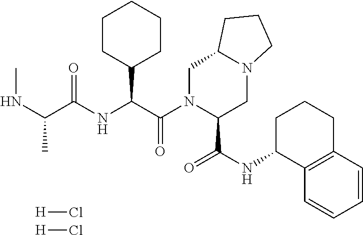

### (i) Production of benzyl[(1S)-2-oxo-2-[(3S,8aS)-3-[(1R)-1,2,3,4-tetrahydronaphthalen-1-ylcarbamoyl]hexahydropyrrolo[1,2-a]pyrazin-2(1H)-yl]-1-(tetrahydro-2H-pyran-4-yl)ethyl]carbamate

A solution (6 mL) of (3S,8aS)—N-[(1R)-1,2,3,4-tetrahydronaphthalen-1-yl]octahydropyrrolo[1,2-a]pyrazine-3-carboxamide dihydrochloride (190 mg), N-ethyldiisopropylamine (330 mg) and (2S)-{[(benzyloxy)carbonyl]amino}(tetrahydro-2H-pyran-4-yl)ethanoic acid (195 mg) in N,N-dimethylformamide was cooled to 0° C., and O-(7-azabenzotriazol-1-yl)-N,N,N′,N′-tetramethyluronium hexafluorophosphate (291 mg) was added thereto. The reaction mixture was allowed to warm to room temperature and stirred for 3 hr. To the reaction mixture was added water (30 mL), and the mixture was diluted with ethyl acetate (150 mL). The organic layer was dried over anhydrous magnesium sulfate, and the insoluble material was filtered off. The filtrate was concentrated under reduced pressure, and the residue was purified by silica gel column chromatography (ethyl acetate/hexane=10/90→100/0) to give the title compound (124 mg) as a colorless oil.

LC-MS: 575.4 (MH⁺).

### (ii) Production of (3S,8aS)-2-[(2S)-2-[(N-methyl-L-alanyl)amino]-2-(tetrahydro-2H-pyran-4-yl)acetyl]-N-[(1R)-1,2,3,4-tetrahydronaphthalen-1-yl]octahydropyrrolo[1,2-a]pyrazine-3-carboxamide dihydrochloride

Benzyl[(1S)-2-oxo-2-[(3S,8aS)-3-[(1R)-1,2,3,4-tetrahydronaphthalen-1-ylcarbamoyl]hexahydropyrrolo[1,2-a]pyrazin-2(1H)-yl]-1-(tetrahydro-2H-pyran-4-yl)ethyl]carbamate (124 mg) was dissolved in methanol (2 mL), 20% palladium hydroxide-carbon (24 mg, 20 wt %) was added thereto, and the mixture was stirred at room temperature for 3 hr under a hydrogen atmosphere. After nitrogen substitution, the insoluble material was filtered off through celite, and washed with methanol. The filtrate was concentrated under reduced pressure to give an oil. N-(tert-butoxycarbonyl)-L-alanine (75 mg) was dissolved in N,N-dimethylformamide (4 mL), and N-ethyldiisopropylamine (181 mg) and O-(7-azabenzotriazol-1-yl)-N,N,N′,N′-tetramethyluronium hexafluorophosphate (181 mg) were added thereto. To the reaction mixture was added a solution prepared by adding N,N-dimethylformamide (1 mL) to the oil obtained above at 0° C., and the mixture was stirred at room temperature for 2 hr. The reaction mixture was poured into ethyl acetate (50 mL)/water (30 mL), and the organic layer was separated. The organic layer was washed with saturated aqueous sodium hydrogen carbonate (30 mL) and saturated brine (30 mL), dried over anhydrous magnesium sulfate, and filtered. The filtrate was concentrated under reduced pressure, and the residue was purified by silica gel column chromatography (hexane/ethyl acetate=50/50→0/100). The object fractions were collected, the solvent was evaporated under reduced pressure, and the residue was dried in vacuum for 1 hr to give a colorless oil (137 mg). This oil was dissolved in ethyl acetate (3 mL), 4M hydrogen chloride-ethyl acetate solution (12 mL) was added thereto, and the mixture was stirred at room temperature for 1 hr. The precipitated solid was collected by filtration, washed with, ethyl acetate, and dried under reduced pressure to give the title compound (105 mg) as a colorless amorphous powder.

LC-MS: 526.4 (MH⁺).

¹H NMR (DMSO-d₆, 300 MHz): δ 1.35 (6H, m), 1.55-2.02 (11H, m), 2.10-2.33 (1H, m), 2.73 (2H, brs), 2.98-3.17 (1H, m), 3.22-3.30 (2H, m), 3.42-3.76 (3H, m), 3.87 (7H, brs), 4.63-4.72 (1H, m), 4.74-4.87 (1H, m), 4.89-5.03 (1H, m), 7.06-7.28 (4H, m), 8.49-8.67 (1H, m), 8.77-8.96 (2H, m), 9.18-9.45 (1H, m), 10.62-10.89 (1H, m).

### Example 74

### Production of (3S,8aR)-2-{(2S)-2-cyclohexyl-2-[(N-methyl-L-alanyl)amino]acetyl}-N-[(1R)-1-naphthalen-1-ylethyl]octahydropyrrolo[1,2-a]pyrazine-3-carboxamide dihydrochloride

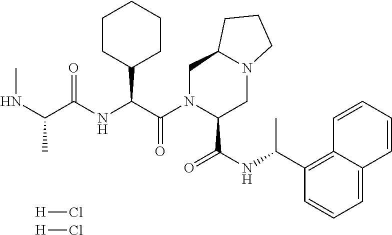

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (8.5 mL), a solution prepared by dissolving lithium hydroxide monohydrate (39 mg) in water (2.1 mL) was added thereto, and the mixture was stirred at 50° C. for 4 hr. To the mixture were added (1R)-1-naphthalen-1-ylethanamine (101 mg) and 4-(4,6-dimethoxy-1,3,5-triazin-2-yl)-4-methylmorpholinium chloride (348 mg), and the mixture was stirred at room temperature for 15 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a colorless amorphous powder. This amorphous powder was dissolved in ethyl acetate (4 mL), 4M hydrogen chloride-ethyl acetate solution (13 mL) was added thereto, and the mixture was stirred at room temperature for 2 hr. The precipitated solid was collected by filtration, washed with ethyl acetate, and dried under reduced pressure to give the title compound (100 mg) as a colorless amorphous powder. LC-MS: 548.4 (MH⁺).

¹H NMR (DMSO-d₆, 300 MHz): δ 0.60-1.31 (6H, m), 1.29-1.42 (4H, m), 1.38-2.25 (14H, m), 2.44 (1H, brs), 3.08 (1H, brs), 3.44-3.95 (5H, m), 4.44 (1H, brs), 4.72 (2H, d, J=7.2 Hz), 5.62 (1H, d, J=7.2 Hz), 7.37-7.57 (3H, m), 7.65 (1H, d, J=7.0 Hz), 7.83 (1H, d, J=8.1 Hz), 7.88-8.01 (1H, m), 8.02-8.17 (1H, m), 8.49-9.05 (3H, m), 9.27 (1H, brs), 12.00 (1H, brs).

### Example 75

### Production of (3S,8aR)-2-{(2S)-2-cyclohexyl-2-[(N-methyl-L-alanyl)amino]acetyl}-N-(6-fluoro-2,3-dihydro-1-benzofuran-3-yl)octahydropyrrolo[1,2-a]pyrazine-3-carboxamide dihydrochloride

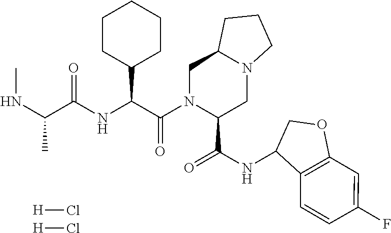

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (300 mg) was dissolved in tetrahydrofuran (9 mL), a solution prepared by dissolving lithium hydroxide monohydrate (39.2 mg) in water (2.2 mL) was added thereto, and the mixture was stirred at 50° C. for 4 hr. To the mixture was added 1M hydrochloric acid (0.93 mL), and the mixture was concentrated under reduced pressure to give a colorless amorphous powder. This amorphous powder, 6-fluoro-2,3-dihydro-1-benzofuran-3-amine hydrochloride (136 mg), 1-ethyl-3-(3-dimethylaminopropyl)carbodiimide hydrochloride (750 mg), 1-hydroxybenzotriazole (80 mg) and N-ethyldiisopropylamine (152 mg) were mixed in N,N-dimethylformamide (12 mL), and the mixture was stirred at room temperature for 16 hr. The mixture was diluted with ethyl acetate (60 mL), and washed with water (15 mL), 10% aqueous sodium hydrogen carbonate solution (15 mL) and saturated brine (30 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in ethyl acetate (3 mL), 4M hydrogen chloride-ethyl acetate solution (13 mL) was added thereto, and the mixture was stirred at room temperature for 2 hr. The precipitated solid was collected by filtration, washed with ethyl acetate, and dried under reduced pressure to give the title compound (125 mg) as a colorless amorphous powder. LC-MS: 530.25 (MH⁺).

¹H NMR (DMSO-d₆, 300 MHz): δ 0.91-1.27 (6H, m), 1.35 (4H, m), 1.70 (9H, brs), 1.99 (3H, s), 3.03 (1H, brs), 3.45-3.76 (5H, m), 3.87 (1H, brs), 4.25-4.65 (3H, m), 4.69-4.83 (2H, m), 5.31-5.52 (1H, m), 6.59-6.83 (2H, m), 7.19-7.36 (1H, m), 8.76 (2H, dd, J=8.0, 4.8 Hz), 8.98-9.08 (1H, m), 9.31 (1H, brs).

### Example 76

### Production of (3S,8aR)-2-{(2S)-2-cyclohexyl-2-[(N-methyl-L-alanyl)amino]acetyl}-N-[(1R)-4-oxo-1,2,3,4-tetrahydronaphthalen-1-yl]octahydropyrrolo[1,2-a]pyrazine-3-carboxamide dihydrochloride

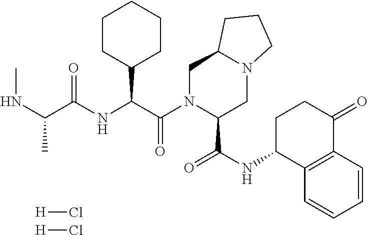

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (200 mg) was dissolved in tetrahydrofuran (6 mL), a solution prepared by dissolving lithium hydroxide monohydrate (24.8 mg) in water (1.5 mL) was added thereto, and the mixture was stirred at 50° C. for 4 hr. To the mixture was added 1M hydrochloric acid (0.62 mL), and the mixture was concentrated under reduced pressure to give a colorless amorphous powder. This amorphous powder, (4R)-4-amino-3,4-dihydronaphthalen-1(2H)-one (95 mg), 1-ethyl-3-3-dimethylaminopropyl)carbodiimide hydrochloride (452 mg), 1-hydroxybenzotriazole (106 mg) and N-ethyldiisopropylamine (0.14 mL) were mixed in N,N-dimethylformamide (5 mL), and the mixture was stirred at room temperature for 16 hr. The mixture was diluted with ethyl acetate (30 mL), and washed with water (30 mL), 10% aqueous sodium hydrogen carbonate solution (30 mL) and saturated brine (30 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in ethyl acetate (2 mL), 4M hydrogen chloride-ethyl acetate solution (8 mL) was added thereto, and the mixture was stirred at room temperature for 2 hr. The precipitated solid was collected by filtration, washed with ethyl acetate, and dried under reduced pressure to give the title compound (105 mg) as a colorless amorphous powder. LC-MS: 538.4 (MH⁺).

¹H NMR (DMSO-d₆, 300 MHz): δ 0.63-1.46 (9H, m), 1.52-2.34 (12H, m), 2.54-2.96 (3H, m), 3.08 (1H, brs), 3.43-4.16 (6H, m), 4.41-4.59 (1H, m), 4.51 (1H, brs), 4.62-4.84 (2H, m), 5.21 (1H, brs), 7.27-7.79 (3H, m), 7.89 (1H, d, J=7.7 Hz), 8.82 (2H, brs), 9.03 (1H, d, J=8.5 Hz), 9.33 (1H, brs), 12.09 (1H, brs).

### Example 77

### Production of (3S,8aR)-2-{(2S)-2-cyclohexyl-2-[(N-methyl-L-alanyl)amino]acetyl}-N-[2,3-dihydro-1-benzofuran-3-yl]octahydropyrrolo[1,2-a]pyrazine-3-carboxamide dihydrochloride

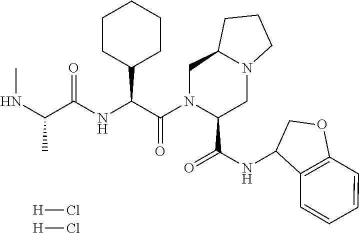

Methyl(3S,8aR)-2-[(2S)-2-{[N-(tert-butoxycarbonyl)-N-methyl-L-alanyl]amino}-2-cyclohexylacetyl]octahydropyrrolo[1,2-a]pyrazine-3-carboxylate (230 mg) was dissolved in tetrahydrofuran (7 mL), a, solution prepared by dissolving lithium hydroxide monohydrate. (30 mg) in water (1.7 mL) was added thereto, and the mixture was stirred at 50° C. for 4 hr. To the mixture was added 1M hydrochloric acid (0.71 mL), and the mixture was concentrated under reduced pressure to give a colorless amorphous powder. This amorphous powder, 2,3-dihydro-1-benzofuran-3-amine hydrochloride (92 mg), O-(7-azabenzotriazol-1-yl)-N,N,N′,N′-tetramethyluronium hexafluorophosphate (258 mg) and N-ethyldiisopropylamine (292 mg) were mixed in N,N-dimethylformamide (5 mL), and the mixture was stirred at room temperature for 16 hr. The mixture was diluted with ethyl acetate (30 mL), and washed with water (30 mL) and saturated brine (30 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in ethyl acetate (4 mL), 4M hydrogen chloride-ethyl acetate solution (8 mL) was added thereto, and the mixture was stirred at room temperature for 2 hr. The precipitated solid was collected by filtration, washed with ethyl acetate, and dried under reduced pressure to give the title compound (121 mg) as a colorless amorphous powder. LC-MS: 512.4 (MH⁺).

¹H NMR (DMSO-d₆, 300 MHz): δ 0.83-1.34 (8H, m), 1.53 (8H, brs), 1.86-2.15 (7H, m), 2.46 (4H, brs), 2.90-3.46 (2H, m), 3.59-4.31 (6H, m), 4.61 (3H, brs), 5.07-5.60 (2H, m), 6.73-6.93 (2H, m), 7.15 (1H, brs).

### Example 78

### Production of (3S,8aR)-2-{(2S)-2-(4,4-difluorocyclohexyl)-2-[(N-methyl-L-alanyl)amino]acetyl}-N-[(1S,2S)-2-hydroxy-2,3-dihydro-1H-inden-1-yl]octahydropyrrolo[1,2-a]pyrazine-3-carboxamide dihydrochloride

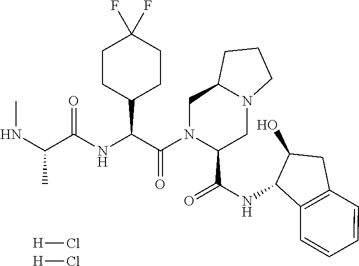

### (i) Production of (3S,8aR)—N-[(1S,2S)-2-hydroxy-2,3-dihydro-1H-inden-1-yl]octahydropyrrolo[1,2-a]pyrazine-3-carboxamide dihydrochloride

2-tert-Butyl 3-methyl(3S,8aR)-hexahydropyrrolo[1,2-a]pyrazine-2,3(1H)-dicarboxylate (7.0 g) was dissolved in tetrahydrofuran (210 mL), a solution prepared by dissolving lithium hydroxide monohydrate (1.7 g) in water (42 mL) was added thereto, and the mixture was stirred at 50° C. for 4 hr. To the mixture was added 1M hydrochloric acid (39 mL), and the mixture was concentrated under reduced pressure to give a colorless amorphous powder. This amorphous powder, (1S,2S)-1-amino-2,3-dihydro-1H-inden-2-ol hydrochloride (5.2 g), 1-ethyl-3-(3-dimethylaminopropyl)carbodiimide hydrochloride (28.3 g), 1-hydroxybenzotriazole (4.3 g) and N-ethyldiisopropylamine (6.4 g) were mixed in N,N-dimethylformamide (400 mL), and the mixture was stirred at room temperature for 16 hr. The mixture was diluted with ethyl acetate (300 mL), and washed with water (300 mL), 5% aqueous sodium hydrogen carbonate solution (300 mL) and saturated brine (150 mL). The organic layer was dried over anhydrous magnesium sulfate, and concentrated under reduced pressure. The residue was purified by basic silica gel column chromatography (ethyl acetate/hexane=20/80→100/0) to give a colorless amorphous powder. This amorphous powder was dissolved in methanol (40 mL), hydrogen chloride-methanol solution (150 mL) was added thereto, and the mixture was stirred at room temperature for 7 hr. The precipitated solid was collected by filtration, washed with ethyl acetate, and dried under reduced pressure to give the title compound (7.2 g) as a colorless amorphous powder. LC-MS: 402.4 (MH⁺).

### (ii) Production of (3S,8aR)-2-[(2S)-2-amino-2-(4,4-difluorocyclohexyl)acetyl]-N-[(1S,2S)-2-hydroxy-2,3-dihydro-1H-inden-1-yl]octahydropyrrolo[1,2-a]pyrazine-3-carboxamide

(3S,8aR)—N-[(1S,2S)-2-Hydroxy-2,3-dihydro-1H-inden-1-yl]octahydropyrrolo[1,2-a]pyrazine-3-carboxamide (598 mg), (2S)-{[(benzyloxy)carbonyl]amino}(4,4-difluorocyclohexyl)ethanoic acid (500 mg), 1-ethyl-3-(3-dimethylaminopropyl)carbodiimide hydrochloride (1.8 g), 1-hydroxybenzotriazole (268 mg), and N,N-diisopropylethylamine (395 mg) were mixed in N,N-dimethylformamide (25 mL), and the mixture was stirred at room temperature for 16 hr. The mixture was diluted with ethyl acetate (70 mL), and washed with water (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give a white solid. This white solid was dissolved in methanol (14 mL), 20% palladium hydroxide (170 mg, 20 wt %) was added thereto, and the mixture was stirred at room temperature for 3 hr under a hydrogen atmosphere. After nitrogen substitution, the insoluble material was filtered off through celite, and washed with methanol. The filtrate was concentrated under reduced pressure to give an oil (340 mg). LC-MS: 477.4 (MH⁺).

### (iii) Production of (3S,8aR)-2-{(2S)-2-(4,4-difluorocyclohexyl)-2-[(N-methyl-L-alanyl)amino]acetyl}-N-[(1S,2S)-2-hydroxy-2,3-dihydro-1H-inden-1-yl]octahydropyrrolo[1,2-a]pyrazine-3-carboxamide dihydrochloride

(3S,8aR)-2-[(2S)-2-Amino-2-(4,4-difluorocyclohexyl)acetyl]-N-[(1S,2S)-2-hydroxy-2,3-dihydro-1H-inden-1-yl]octahydropyrrolo[1,2-a]pyrazine-3-carboxamide (340 mg) and N-(tert-butoxycarbonyl)-N-methyl-L-alanine (189 mg), 1-ethyl-3-(3-dimethylaminopropyl)carbodiimide hydrochloride (821 mg), 1-hydroxybenzotriazole (125 mg) and N,N-diisopropylethylamine (184 mg) were mixed in N,N-dimethylformamide (12 mL), and the mixture was stirred at room temperature for 16 hr. The mixture was diluted with ethyl acetate (50 mL), and washed with water (50 mL), saturated aqueous sodium hydrogen carbonate (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give an oil (181 mg). The obtained oil was dissolved in ethyl acetate (3 mL), 4M hydrogen chloride-ethyl acetate solution (10 mL) was added thereto, and the mixture was stirred at room temperature for 2 hr. The precipitated solid was collected by filtration, washed with ethyl acetate, and dried under reduced pressure to give the title compound (156 mg) as a colorless amorphous powder.

LC-MS: 562.3 (MH⁺).

¹H NMR (DMSO-d₆, 300 MHz): δ 1.25-1.49 (6H, m), 1.62-2.23 (13H, m), 2.73 (1H, brs), 2.99-3.20 (3H, m), 3.51-4.03 (6H, m), 4.12-4.33 (1H, m), 4.47-4.75 (2H, m), 4.93 (2H, d, J=7.6 Hz), 5.35 (1H, brs), 6.93-7.50 (4H, m), 8.60-9.10 (3H, m), 9.45 (1H, brs).

### Example 79

### Production of (3S,8aR)-2-{(2S)-2-cyclohexyl-2-[(N-methyl-L-alanyl)amino]acetyl}-N-(2,3-dihydro-1H-indol-1-yl)octahydropyrrolo[1,2-a]pyrazine-3-carboxamide dihydrochloride

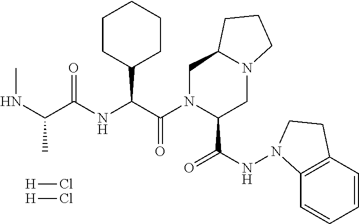

### (i) Production of (3S,8aR)—N-(2,3-dihydro-1H-indol-1-yl)octahydropyrrolo[1,2-a]pyrazine-3-carboxamide trihydrochloride

2-tert-Butyl 3-methyl(3S,8aR)-hexahydropyrrolo[1,2-a]pyrazine-2,3(1H)-dicarboxylate (1.5 g) was dissolved in tetrahydrofuran (75 mL), a solution prepared by dissolving lithium hydroxide monohydrate (360 mg) in water (30 mL) was added thereto, and the mixture was stirred at 50° C. for 4 hr. To the mixture was added 1M hydrochloric acid (8.4 mL), and the mixture was concentrated under reduced pressure to give a colorless amorphous powder. This amorphous powder, 2,3-dihydro-1H-indol-1-amine hydrochloride (1.06 g), 1-ethyl-3-(3-dimethylaminopropyl)carbodiimide hydrochloride (6.7 g), 1-hydroxybenzotriazole (856 mg) and N,N-diisopropylethylamine (1.36 g) were mixed in N,N-dimethylformamide (100 mL), and the mixture was stirred at room temperature for 16 hr. The mixture was diluted with ethyl acetate (200 mL), and washed with water (150 mL) and saturated brine (100 mL). The organic layer was dried over anhydrous magnesium sulfate, and concentrated under reduced pressure. The residue was purified by silica gel column chromatography (ethyl acetate/hexane=0/100→100/0) to give a white solid. This white solid was dissolved in ethyl acetate (15 mL), 4M hydrogen chloride-ethyl acetate solution (45 mL) was added thereto, and the mixture was stirred at room temperature for 2 hr. The precipitated solid was collected by filtration, washed with ethyl acetate, and dried under reduced pressure to give the title compound (1.54 g) as a white solid.

LC-MS: 287.2 (MH⁺).

### (ii) Production of (2S)-({N-[(benzyloxy)carbonyl]-N-methyl-L-alanyl}amino) (cyclohexyl)ethanoic acid

To a solution of N-[(benzyloxy)carbonyl]-N-methyl-L-alanine (4.93 g), methyl(2S)-amino(cyclohexyl)ethanoate (4.32 g) and 2-chloro-4,6-dimethoxy-1,3,5-triazine (3.83 g) in ethyl acetate (75 mL) was added 4-methylmorpholine (5.71 mL) under ice-cooling, and the mixture was stirred at 0° C. for 2 hr. The insoluble material was filtered off, and the filtrate was washed with saturated aqueous sodium hydrogen carbonate solution (50 mL), 10% aqueous citric acid solution (50 mL) and saturated brine (50 mL), and dried over anhydrous magnesium sulfate. The insoluble material was filtered off. The filtrate was concentrated under reduced pressure, and the residue was subjected to silica gel chromatography (hexane/ethyl acetate=95/5→50/50), and the collected fractions were concentrated to give methyl(2S)-({N-[(benzyloxy)carbonyl]-N-methyl-L-alanyl}amino)(cyclohexyl)ethanoate (8.20 g). This methyl(2S)-({N-[(benzyloxy)carbonyl]-N-methyl-L-alanyl}amino) (cyclohexyl)ethanoate (8.20 g) was dissolved in tetrahydrofuran (75 mL), an aqueous solution (75 mL) of lithium hydroxide monohydrate (1.15 g) was added thereto, and the mixture was stirred at room temperature for 4 hr. To the reaction mixture was added 10% aqueous citric acid solution (80 mL), and tetrahydrofuran was evaporated under reduced pressure. The residue was extracted with ethyl acetate (200 mL×2), and the organic layer was washed with saturated brine, and dried over anhydrous magnesium sulfate. The insoluble material was filtered off. The filtrate was concentrated under reduced pressure, and the residue was crystallized from diisopropyl ether (50 mL)/hexane (200 mL) to give the title compound (6.79 g) as colorless crystals. LC-MS: 377.3(M+H⁺).

¹H NMR (DMSO-d₆, 400 MHz): δ 0.90-1.33 (8H, m), 1.45-1.75 (6H, m), 2.84 (3H, brs), 4.08 (1H, t, J=7.3 Hz), 4.69 (1H, brs), 5.07 (2H, d, J=12.2 Hz), 7.18-7.45 (5H, m), 7.81-8.05 (1H, m), 12.56 (1H, brs).

### (iii) Production of benzyl[(1S)-2-({(1S)-1-cyclohexyl-2-[(3S,8aR)-3-(2,3-dihydro-1H-indol-1-ylcarbamoyl)hexahydropyrrolo[1,2-a]pyrazin-2(1H)-yl]-2-oxoethyl}amino)-1-methyl-2-oxoethyl]methylcarbamate

(3S,8aR)—N-(2,3-Dihydro-1H-indol-1-yl)octahydropyrrolo[1,2-a]pyrazine-3-carboxamide trihydrochloride (625 mg), (2S)-({N-[(benzyloxy)carbonyl]-N-methyl-L-alanyl}amino)(cyclohexyl)ethanoic acid (1.05 g) and O-(7-azabenzotriazol-1-yl)-N,N,N′,N′ tetramethyluronium hexafluorophosphate (1.03 g) were mixed in tetrahydrofuran (30 mL), and the mixture was stirred at room temperature for 16 hr. The mixture was diluted with ethyl acetate (50 mL), and washed with water (50 mL) and saturated brine (50 mL). The organic layer was dried over anhydrous magnesium sulfate, and concentrated under reduced pressure. The residue was purified by silica gel column chromatography (ethyl acetate/hexane=30/70→100/0) and then basic silica gel column chromatography (ethyl acetate/hexane=5/95→50/50) to give the title compound (140 mg) as a white solid. LC-MS: 645.4 (MH⁺)

### (iv) Production of (3S,8aR)-2-{(2S)-2-cyclohexyl-2-[(N-methyl-L-alanyl)amino]acetyl}-N-(2,3-dihydro-1H-indol-1-yl)octahydropyrrolo[1,2-a]pyrazine-3-carboxamide trihydrochloride

Benzyl[(1S)-2-({(1S)-1-cyclohexyl-2-[(3S,8aR)-3-(2,3-dihydro-1H-indol-1-ylcarbamoyl)hexahydropyrrolo[1,2-a]pyrazin-2(1H)-yl]-2-oxoethyl}amino)-1-methyl-2-oxoethyl]-methylcarbamate (140 mg) was dissolved in methanol (3 mL), 20% palladium hydroxide (26 mg) was added thereto, and the mixture was stirred at room temperature for 3 hr under a hydrogen atmosphere. After nitrogen substitution, the insoluble material was filtered off through celite, and washed with methanol. The filtrate was concentrated under reduced pressure to give an oil. The obtained oil (105 mg) was dissolved in 4M hydrogen chloride-ethyl acetate solution (8 mL), and the mixture was stirred at room temperature for 1 hr. The precipitated solid was collected by filtration, washed with ethyl acetate, and dried under reduced pressure to give the title compound (117 mg) as a colorless amorphous powder.

LC-MS: 511.4 (MH⁺).

¹H NMR (DMSO-d₆, 300 MHz): δ 0.79-1.43 (13H, m), 1.51-1.87 (7H, m), 1.99-2.40 (2H, m), 2.41-2.47 (2H, m), 2.54-3.31 (4H, m), 3.41-4.01 (6H, m), 4.42-5.08 (2H, m), 6.10-7.78 (4H, m), 8.19-9.12 (2H, m), 9.37 (1H, brs), 10.08-12.45 (1H, m).

### Example 80

### Production of ((3S,7R,8aS)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

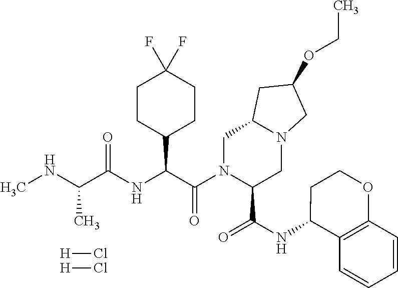

### (i) Production of (4R)-1-(tert-butoxycarbonyl)-4-ethoxy-L-proline

To a mixture of (4R)-1-(tert-butoxycarbonyl)-4-hydroxy-L-proline (10.0 g) in tetrahydrofuran (150 mL) was added sodium hydride (13.0 g) at room temperature, and the mixture was stirred at 60° C. for 1 hr. The reaction mixture was allowed to cool to room temperature, ethyl iodide (17.3 mL) was added thereto, and the mixture was stirred at room temperature for 18 hr. To the mixture were added water (50 mL), 1M hydrochloric acid (50 mL) and sodium chloride (about 30 g), and the solution was extracted with ethyl acetate (250 mL×2). The organic layer was dried over anhydrous magnesium sulfate, and the insoluble material was filtered off. The filtrate was concentrated under reduced pressure, and the residue was subjected to silica gel chromatography (hexane/ethyl acetate=50/50→ethyl acetate→ethyl acetate/methanol=90/10), and the collected fractions were concentrated to give the title compound (10.94 g) as a colorless oil.

¹H NMR (DMSO-d₆, 300 MHz): δ 1.09 (3H, t, J=7.0 Hz), 1.31-1.44 (9H, m), 1.86-2.05 (1H, m), 2.14-2.35 (1H, m), 3.23-3.52 (4H, m), 3.98-4.14 (2H, m), 12.57 (1H, brs).

### (ii) Production of tert-butyl(2S,4R)-2-(benzylcarbamoyl)-4-ethoxypyrrolidine-1-carboxylate

To a suspension (175 mL) of (4R)-1-(tert-butoxycarbonyl)-4-ethoxy-L-proline (10.5 g), benzylamine (4.87 mL) and 1-hydroxybenzotriazole (6.02 g) in acetonitrile was added 1-ethyl-3-(3-dimethylaminopropyl)carbodiimide hydrochloride (11.64 g) under ice-cooling, and the mixture was allowed to warm to room temperature, and stirred for 1 hr. To this reaction mixture was added water (100 mL), and the mixture was extracted with ethyl acetate (500 mL). The organic layer was washed with saturated aqueous sodium hydrogen carbonate solution (100 mL×2), and the insoluble material was filtered off. The filtrate was concentrated under reduced pressure, the residue was subjected to silica gel chromatography (hexane/ethyl acetate=90/10→90/10), and the collected fractions were concentrated to give the title compound (12.84 g) as a pale-yellow oil. LC-MS: 349.2 (MH⁺).

¹H NMR (DMSO-d₆, 300 MHz): δ 1.09 (3H, t, J=7.0 Hz), 1.23-1.46 (9H, m), 1.82-1.96 (1H, m), 2.09-2.31 (1H, m), 3.35-3.52 (4H, m), 3.90-4.43 (4H, m), 7.16-7.38 (5H, m), 8.34-8.51 (1H, m).

### (iii) Production of (4R)—N-benzyl-4-ethoxy-L-prolinamide

To a solution of tert-butyl(2S,4R)-2-(benzylcarbamoyl)-4-ethoxypyrrolidine-1-carboxylate (12.5 g) in ethyl acetate (50 mL) was added 4M hydrogen chloride-ethyl acetate solution (50 mL), and the mixture was stirred at room temperature for 2 hr, and concentrated. The residue was dissolved in methanol (20 mL), the solution was filtered off through a pad filled with Amberlyst A21 (trade name) (50 g), and the pad was washed with methanol (250 mL). The filtrate was concentrated to give the title compound as a crude product (9.37 g). Without further purification, the compound was used for the next reaction. LC-MS: 249.2 (MH⁺).

### (iv) Production of N-benzyl-1-[(2S,4R)-4-ethoxypyrrolidin-2-yl]methanamine

(4R)—N-Benzyl-4-ethoxy-L-prolinamide (7.22 g) in tetrahydrofuran solution (50 mL) was added to a suspension (300 mL) of lithium aluminum hydride (2.76 g) in tetrahydrofuran under ice-cooling, and the reaction mixture was heated under reflux for 24 hr. The reaction mixture was cooled to 0° C., and sodium sulfate decahydrate (2.7 g) was added thereto. The insoluble material was filtered off through a celite pad, and the filtrate was concentrated under reduced pressure to give a colorless oil (5.51 g). Without further purification, the compound was used for the next reaction. LC-MS: 235.1 (MH⁺).

### (v) Production of methyl(3S,7R,8aS)-2-benzyl-7-ethoxyoctahydropyrrolo[1,2-a]pyrazine-3-carboxylate

To a solution of N-benzyl-1-[(2S,4R)-4-ethoxypyrrolidin-2-yl]methanamine (5.00 g) and methyl 2,3-dibromopropanoate (4.46 g) in toluene (75 mL) was added triethylamine (2.97 mL), and the mixture was stirred at 80° C. for 3 hr. The reaction mixture was allowed to cool to room temperature, and the mixture was diluted with ethyl acetate (300 mL) and water (100 mL). The organic layer was dried over anhydrous magnesium sulfate, the insoluble material was filtered off, and the filtrate was concentrated under reduced pressure. The residue was purified by silica gel column chromatography (ethyl acetate/hexane=10/90→100/0) to give the title compound (2.56 g) as a yellow oil. LC-MS: 319.2 (MH⁺).

¹H NMR (DMSO-d₆, 300 MHz): δ 1.05 (3H, t, J=7.0 Hz), 1.36-1.51 (1H, m), 1.56-1.67 (1H, m), 1.68-1.79 (1H, m), 1.95-2.04 (1H, m), 2.12-2.31 (2H, m), 2.68-2.78 (1H, m), 3.01-3.20 (3H, m), 3.23-3.43 (3H, m), 3.66 (3H, s), 3.69-3.78 (1H, m), 3.87-4.01 (1H, m), 7.19-7.39 (5H, m).

### (vi) Production of 2-tert-butyl 3-methyl(3S,7R,8aS)-7-ethoxyhexahydropyrrolo[1,2-a]pyrazine-2,3(1H)-dicarboxylate

Methyl(3S,7R,8aS)-2-benzyl-7-ethoxyoctahydropyrrolo[1,2-a]pyrazine-3-carboxylate (2.10 g) was dissolved in 5-10% hydrogen chloride-methanol solution (20 mL), and 10% palladium carbon (210 mg) was added thereto. The reaction mixture was stirred at room temperature for 3 hr under a hydrogen atmosphere (3 atm). The insoluble material was filtered off through a celite pad, and the filtrate was concentrated to give a pale-yellow oil (2.02 g). This oil was dissolved in saturated aqueous sodium hydrogen carbonate solution (15 mL) and tetrahydrofuran (15 mL), to the resulting two-phase mixture was added di-tert-butyl bicarbonate (1.57 g), and the mixture was stirred at room temperature for 2 hr. The mixture was diluted with ethyl acetate (300 mL), and washed with saturated brine (100 mL). The organic layer was dried over anhydrous magnesium sulfate, the insoluble material was filtered off, and the filtrate was concentrated. The residue was purified by silica gel column chromatography (hexane→ethyl acetate) to give the title compound (1.82 g) as a colorless oil. LC-MS: 329.2 (MH⁺).

¹H NMR (DMSO-d₆, 300 MHz): δ 1.08 (3H, t, J=7.0 Hz), 1.36 (9H, s), 1.52-1.69 (1H, m), 1.74-1.88 (1H, m), 2.57 (1H, brs), 2.71-3.04 (3H, m), 3.07-3.19 (1H, m), 3.26-3.45 (3H, m), 3.54-3.63 (1H, m), 3.65 (3H, s), 3.91-4.10 (1H, m), 4.19-4.32 (1H, m).

### (vii) Production of tert-butyl(3S,7R,8aS)-3-[(4R)-3,4-dihydro-2H-chromen-4-ylcarbamoyl]-7-ethoxyhexahydropyrrolo[1,2-a]pyrazine-2(1H)-carboxylate

To a solution of 2-tert-butyl 3-methyl(3S,7R,8aS)-7-ethoxyhexahydropyrrolo[1,2-a]pyrazine-2,3(1H)-dicarboxylate (1.6 g) in tetrahydrofuran (25 mL)/water (5 mL) was added lithium hydroxide monohydrate (266 mg) at room temperature, and the reaction mixture was stirred at 50° C. for 3 hr. The reaction mixture was allowed to cool to room temperature, neutralized with 1M hydrochloric acid (6.5 mL), and concentrated under reduced pressure. The residue was subjected to azeotropic distillation with toluene (25 mL), and dried in vacuum. To a solution of the obtained residue in N,N-dimethylformamide (25 mL) was added N,N-diisopropylethylamine (2.54 mL), and the mixture was cooled to 0° C. 1-Hydroxybenzotriazole (790 mg) and 1-ethyl-3-(3-dimethylaminopropyl)carbodiimide hydrochloride (2.80 g) were added thereto, and the mixture was stirred at room temperature for 30 min. Then, (4R)-3,4-Hihydro-2H-chromen-4-amine hydrochloride (949 mg) was added thereto, and the reaction mixture was stirred at room temperature for 72 hr, and diluted with ethyl acetate (350 mL) and water (100 mL). The organic layer was dried over anhydrous magnesium sulfate, the insoluble material was filtered off, and the filtrate was concentrated under reduced pressure. The residue was purified by silica gel column chromatography (ethyl acetate/hexane=5/95→100/0) to give the title compound (1.25 g) as a colorless amorphous solid. LC-MS: 446.3 (MH⁺).

¹H NMR (DMSO-d₆, 300 MHz): δ 1.10 (3H, t, J=7.0 Hz), 1.38 (9H, s), 1.48-1.63 (1H, m), 1.75-2.08 (3H, m), 2.62-2.76 (1H, m), 2.78-2.99 (3H, m), 3.06-3.23 (1H, m), 3.27-3.33 (1H, m), 3.38 (2H, q, J=7.0 Hz), 3.48-3.63 (1H, m), 3.88-4.04 (1H, m), 4.19 (3H, t, J=5.2 Hz), 5.01 (1H, q, J=7.0 Hz), 6.75 (1H, d, J=8.1 Hz), 6.80-6.88 (1H, m), 7.05-7.25 (2H, m), 8.30 (1H, d, J=8.1 Hz).

### (viii) Production of (3S,7R,8aS)—N-[(4R)-3,4-dihydro-2H-chromen-4-yl]-7-ethoxyoctahydropyrrolo[1,2-a]pyrazine-3-carboxamide

To a solution of tert-butyl(3S,7R,8aS)-3-[(4R)-3,4-dihydro-2H-chromen-4-ylcarbamoyl]-7-ethoxyhexahydropyrrolo[1,2-a]pyrazine-2(1H)-carboxylate (1.00 g) in methanol (5 mL) was added 4M hydrogen chloride-ethyl acetate solution (5 mL), and the mixture was stirred at room temperature for 4 hr, and concentrated. The residue was dissolved in methanol (10 mL), the solution was filtered off through a pad filled with Amberlyst A21 (trade name) (50 g), the pad was washed with methanol (100 mL), and the filtrate was concentrated. The residue was purified by silica gel column chromatography (ethyl acetate→methanol/ethyl acetate=20/80) to give the title compound (586 mg) as a colorless amorphous solid. LC-MS: 346.2 (MH⁺).

¹H NMR (DMSO-d₆, 300 MHz): δ 1.08 (3H, t, J=7.0 Hz), 1.38-1.56 (1H, m), 1.63-1.74 (1H, m), 1.79-2.29 (5H, m), 2.38 (1H, t, J=11.2 Hz), 3.12 (2H, ddd, J=18.5, 11.5, 2.9 Hz), 3.23-3.58 (4H, m), 3.90-4.00 (1H, m), 4.11-4.26 (2H, m), 4.88-5.10 (1H, m), 5.80 (1H, brs), 6.72-6.81 (1H, m), 6.84-6.92 (1H, m), 7.07-7.21 (2H, m), 8.19-8.61 (1H, m).

### (ix) Production of benzyl{(1S)-1-(4,4-difluorocyclohexyl)-2-[(3S,7R,8aS)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate

A solution of (2S)-{[(benzyloxy)carbonyl]amino}(4,4-difluorocyclohexyl)ethanoic acid (469 mg), 1-hydroxybenzotriazole (211 mg), N,N-diisopropylethylamine (0.681 mL) and 1-ethyl-3-(3-dimethylaminopropyl)carbodiimide hydrochloride (749 mg) in N,N-dimethylformamide (5 mL) was stirred at room temperature for 15 min. To the reaction mixture was added (3S,7R,8aS)—N-[(4R)-3,4-dihydro-2H-chromen-4-yl]-7-ethoxyoctahydropyrrolo[1,2-a]pyrazine-3-carboxamide (450 mg), and the mixture was stirred at room temperature for 12 hr, and the mixture was diluted with ethyl acetate (350 mL) and water (100 mL). The organic layer was washed with saturated brine, and dried over anhydrous magnesium sulfate, the insoluble material was filtered off, and the filtrate was concentrated under reduced pressure. The residue was purified by silica gel column chromatography (ethyl acetate→methanol/ethyl acetate=10/90) to give the title compound (514 mg) as a colorless amorphous solid. LC-MS: 655.4 (MH⁺).

¹H NMR (DMSO-d₆, 300 MHz): δ 1.10 (3H, t, J=7.0 Hz), 1.21-1.42 (2H, m), 1.46-2.14 (10H, m), 2.66-3.10 (3H, m), 3.21-3.48 (6H, m), 3.61-3.81 (1H, m), 3.87-3.97 (1H, m), 4.10-4.26 (2H, m), 4.33-4.53 (2H, m), 4.78-5.20 (3H, m), 6.75 (1H, d, J=8.1 Hz), 6.80-6.89 (1H, m), 7.08-7.17 (1H, m), 7.17-7.24 (1H, m), 7.25-7.42 (5H, m), 7.58 (1H, d, J=8.5 Hz), 8.21 (1H, d, J=8.7 Hz).

### (x) Production of tert-butyl[(1S)-2-({(1S)-1-(4,4-difluorocyclohexyl)-2-[(3S,7R,8aS)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate

Benzyl{(1S)-1-(4,4-difluorocyclohexyl)-2-[(3S,7R,8aS)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (400 mg) was dissolved in 5-10% hydrogen chloride-methanol solution (4 mL), 20% palladium hydroxide carbon (80 mg) was added thereto, and the mixture was stirred at room temperature for 3 hr under a hydrogen atmosphere (1 atm). The insoluble material was filtered off through a celite pad, and the filtrate was concentrated to give pale-yellow amorphous solid. The obtained amorphous solid (400 mg), 1-hydroxybenzotriazole (99.1 mg), N,N-diisopropylethylamine (0.532 mL) and N-(tert-butoxycarbonyl)-N-methyl-L-alanine (137 mg) were dissolved in N,N-dimethylformamide (4 mL). The mixture was cooled to 0° C., 1-ethyl-3-(3-dimethylaminopropyl)carbodiimide hydrochloride (351 mg) was added thereto, and the mixture was stirred at room temperature for 4 hr. The reaction mixture was diluted with ethyl acetate (300 mL) and water (100 mL). The organic layer was washed with saturated aqueous sodium hydrogen carbonate solution (100 mL) and saturated brine (100 mL), and dried over anhydrous magnesium sulfate, the insoluble material was filtered off, and the filtrate was concentrated under reduced pressure. The residue was purified by silica gel column chromatography (ethyl acetate→methanol/ethyl acetate=10/90) to give the title compound (329 mg) as a colorless amorphous solid. LC-MS: 706.4 (MH⁺).

¹H NMR (DMSO-d₆, 300 MHz): δ 1.04-1.15 (3H, m), 1.16-1.33 (5H, m), 1.39 (9H, s), 1.47-2.08 (12H, m), 2.66-2.85 (5H, m), 2.87-3.07 (2H, m), 3.22-3.47 (4H, m), 3.65-3.85 (1H, m), 3.90-4.04 (1H, m), 4.13-4.22 (1H, m), 4.33-4.46 (1H, m), 4.47-4.59 (1H, m), 4.62-4.75 (1H, m), 4.86-5.11 (1H, m), 6.70-6.78 (1H, m), 6.80-6.93 (1H, m), 7.06-7.18 (1H, m), 7.18-7.26 (1H, m), 7.38-7.99 (1H, m), 8.14-8.49 (1H, m).

### (xi) Production of (3S,7R,8aS)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

To a solution of tert-butyl[(1S)-2-({(1S)-1-(4,4-difluorocyclohexyl)-2-[(3S,7R,8aS)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (190 mg) in ethyl acetate (3 mL) was added 4M hydrogen chloride-ethyl acetate solution (3 mL), and the reaction mixture was stirred at room temperature for 12 hr. To the mixture was added diethyl ether (15 mL), and the precipitate was collected by filtration, washed with ethyl acetate, and dried to give the title compound. (175 mg) as a colorless amorphous solid. LC-MS: 606.3 (MH⁺).

¹H NMR (DMSO-d₆, 300 MHz): δ 1.10-1.18 (3H, m), 1.28-1.49 (5H, m), 1.62-2.15 (8H, m), 2.18-2.32 (1H, m), 2.39-2.50 (3H, m), 3.19-3.75 (8H, R), 3.82-4.38 (7H, m), 4.56-4.84 (2H, m), 4.89-5.13 (1H, m), 6.68-6.96 (2H, m), 7.04-7.63 (2H, m), 8.60-9.07 (3H, m), 9.19-9.67 (1H, m), 10.85-11.42 (1H, m).

### Example 81

### Production of (3S,7S,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

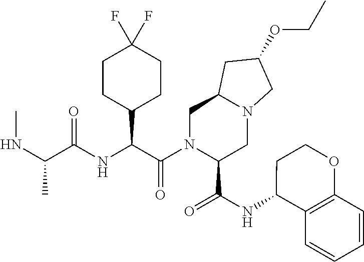

### (i) Production of 2-tert-butyl 3-methyl(3S,7S,8aR)-7-hydroxyhexahydropyrrolo[1,2-a]pyrazine-2,3(1H)-dicarboxylate

Diisopropyl azodicarboxylate (270 mg) and triphenylphosphine (350 mg) were dissolved in dry tetrahydrofuran (1 mL), and the solution was stirred at 0° C. for 30 min. A solution (1 mL) of 2-tert-butyl 3-methyl(3S,7R,8aR)-7-hydroxyhexahydropyrrolo[1,2-a]pyrazine-2,3(1H)-dicarboxylate (200 mg) and formic acid (30 mg) in dry tetrahydrofuran was added thereto, and the mixture was stirred for 1.5 hr, and concentrated under reduced pressure. The residue was purified by silica gel column chromatography (ethyl acetate/hexane=0/100→80/20) to give a yellow oil. This yellow oil was dissolved in methanol (3 mL), 1M aqueous sodium hydroxide solution (0.7 mL) was added thereto under ice-cooling, and the mixture was stirred for 10 min. The mixture was neutralized with 1M hydrochloric acid, and extracted with ethyl acetate (15 mL)-tetrahydrofuran (15 mL). The organic layer was dried over anhydrous magnesium sulfate, and concentrated under reduced pressure, and the residue was purified by silica gel column chromatography (ethyl acetate/hexane=0/100→100/0) to give the title compound (160 mg) as a yellow oil. LC-MS: 301.2 (MH⁺).

### (ii) Production of tert-butyl(3S,7S,8aR)-3-[(4R)-3,4-dihydro-2H-chromen-4-ylcarbamoyl]-7-ethoxyhexahydropyrrolo[1,2-a]pyrazine-2(1H)-carboxylate

To a suspension of 2-tert-butyl 3-methyl(3S,7S,8aR)-7-hydroxyhexahydropyrrolo[1,2-a]pyrazine-2,3(1H)-dicarboxylate (800 mg) in tetrahydrofuran (12 mL) was added sodium hydride (60%, 320 mg) under ice-cooling. The mixture was stirred at room temperature for 30 min, bromoethane (1.40 g) was added thereto, and the mixture was stirred at 60° C. for 18 hr. The reaction, mixture was allowed to cool to room temperature, diluted with methanol (15 mL) and ethyl acetate (50 mL), and concentrated under reduced pressure to give a yellow oil. This oil, (4R)-3,4-dihydro-2H-chromen-4-amine hydrochloride (868 mg), 1-ethyl-3-(3-dimethylaminopropyl)carbodiimide hydrochloride (1.30 g), 1-hydroxybenzotriazole (470 mg) and N,N-diisopropylethylamine (910 mg) were mixed in N,N-dimethylformamide (15 mL), and the mixture was stirred at roam temperature for 18 hr. The mixture was diluted with ethyl acetate (100 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→80/20) to give the title compound (881% mg) as a colorless oil. LC-MS: 446.3 (MH⁺).

### (iii) Production of benzyl{(1S)-1-(4,4-difluorocyclohexyl)-2-[(3S,7S,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate

tert-Butyl(3S,7S,8aR)-3-[(4R)-3,4-dihydro-2H-chromen-4-ylcarbamoyl]-7-ethoxyhexahydropyrrolo[1,2-a]pyrazine-2(1H)-carboxylate (881 mg) was dissolved in methanol (5 mL), 4M hydrogen chloride-cyclopentyl methyl ether solution (18 mL) was added thereto, and the mixture was stirred at room temperature for 3 hr. The reaction mixture was concentrated under reduced pressure, and the precipitated solid was collected by filtration, and washed with ethyl acetate to give colorless amorphous solid. This amorphous solid, (2S)-{[(benzyloxy)carbonyl]amino}(4,4-difluorocyclohexyl)ethanoic acid (620 mg), 1-ethyl-3-(3-dimethylaminopropyl)carbodiimide hydrochloride (1.68 g), 1-hydroxybenzotriazole (256 mg) and N,N-diisopropylethylamine (471 mg) were mixed in N,N-dimethylformamide (25 mL), and the mixture was stirred at room temperature for 18 hr. The mixture was diluted with ethyl acetate (100 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (520 mg) as a yellow amorphous solid. LC-MS: 655.4 (MH⁺).

### (iv) Production of tert-butyl[(1S)-2-({(1S)-1-(4,4-difluorocyclohexyl)-2-[(3S,7S,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate

Benzyl{(1S)-1-(4,4-difluorocyclohexyl)-2-[(3S,7S,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (520 mg) was dissolved in methanol (2.5 mL), 20% palladium hydroxide (106 mg, 20 wt %) was added thereto, and the mixture was stirred at room temperature for 3 hr under a hydrogen atmosphere. After nitrogen substitution, the insoluble material was filtered off through celite, and washed with methanol. The filtrate was concentrated under reduced pressure to give a yellow oil. This yellow oil, N-(tert-butoxycarbonyl)-N-methyl-L-alanine (150 mg), 1-ethyl-3-(3-dimethylaminopropyl)carbodiimide hydrochloride (236 mg), 1-hydroxybenzotriazole (91 mg) and N,N-diisopropylethylamine (238 mg) were mixed in N,N-dimethylformamide (5 mL), and the mixture was stirred at room temperature for 16 hr. The mixture was diluted with ethyl acetate (50 mL), and washed with water (30 mL), 10% aqueous sodium hydrogen carbonate solution (30 mL) and saturated brine (20 mL). The organic layer was dried over anhydrous magnesium sulfate, and concentrated under reduced pressure. The residue was purified by silica gel column chromatography (ethyl acetate/hexane=0/100→100/0) to give the title compound (345 mg) as an oil. LC-MS: 706.4 (MH⁺).

### (v) Production of (3S,7S,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

tert-Butyl[(1S)-2-({(1S)-1-(4,4-difluorocyclohexyl)-2-[(3S,7S,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (345 mg) was dissolved in methanol (3 mL), 4M hydrogen chloride-cyclopentyl methyl ether solution (12 mL) was added thereto, and the mixture was stirred at room temperature for 2 hr. To the mixture was added water (20 mL), and the aqueous layer was separated. The aqueous layer was neutralized with 1M aqueous sodium hydroxide solution, and extracted with ethyl acetate (10 mL). The organic layer was washed with saturated brine (10 mL), dried over anhydrous magnesium sulfate, and concentrated under reduced pressure. The residue was dissolved in diethyl ether (1 mL), heptane (3 mL) was added thereto, and the mixture was stirred at room temperature for 15 min. The precipitated solid was collected by filtration, and washed with ethyl acetate-hexane to give the title compound (126 mg) as a white solid. LC-MS: 606.4 (MH⁺).

¹H NMR (CDCl₃ 300 MHz): δ 1.14-1.34 (8H, m), 1.49-2.52 (18H, m), 2.86-3.16 (2H, m), 3.30-3.93 (4H, m), 3.98-4.15 (2H, m), 4.17-4.33 (1H, m), 4.48-5.34 (3H, m), 6.27 (1H, brs), 6.72-7.01 (2H, m), 7.10-7.43 (3H, m), 7.54-7.99 (1H, m).

### Example 82

### Production of (3S,7R,8aR)-2-{(2S)-2-cyclohexyl-2-[(N-methyl-L-alanyl)amino]acetyl}-7-(cyclopropylmethoxy)-N-[(4R)-3,4-dihydro-2H-chromen-4-yl]octahydropyrrolo[1,2-a]pyrazine-3-carboxamide

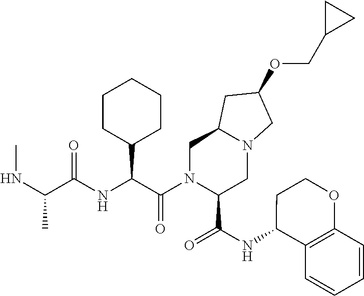

### (i) Production of tert-butyl(3S,7R,8aR)-7-(cyclopropylmethoxy)-3-[(4R)-3,4-dihydro-2H-chromen-4-ylcarbamoyl]hexahydropyrrolo[1,2-a]pyrazine-2(1H)-carboxylate

To a suspension of 2-tert-butyl 3-methyl(3S,7R,8aR)-7-hydroxyhexahydropyrrolo[1,2-a]pyrazine-2,3(1H)-dicarboxylate (1.05 g) in tetrahydrofuran (14 mL) was added sodium hydride (60%, 600 mg) under ice-cooling. The mixture was stirred at room temperature for 30 min, (bromomethyl)cyclopropane (1.64 g) was added thereto, and the mixture was stirred at 60° C. for 18 hr. The reaction mixture was allowed to cool to room temperature, diluted with methanol (10 mL) and ethyl acetate (30 mL), and concentrated under reduced pressure to give a yellow oil. This yellow oil, (4R)-3,4-dihydro-2H-chromen-4-amine hydrochloride (520 mg), 1-ethyl-3-(3-dimethylaminopropyl)carbodiimide hydrochloride (805 mg), 1-hydroxybenzotriazole (284 mg) and N,N-diisopropylethylamine (543 mg) were mixed in N,N-dimethylformamide (10 mL), and the mixture was stirred at room temperature for 18 hr. The mixture was diluted with ethyl acetate (70 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (843 mg) as an oil. LC-MS: 472.3 (MH⁺).

### (ii) Production of tert-butyl{(1S)-1-cyclohexyl-2-[(3S,7R,8aR)-7-(cyclopropylmethoxy)-3-[(4R)-3,4-dihydro-2H-chromen-4-ylcarbamoyl]hexahydropyrrolo[1,2-a]pyrazin-2(1H)-yl]-2-oxoethyl}carbamate

tert-Butyl(3S,7R,8aR)-7-(cyclopropylmethoxy)-3-[(4R)-3,4-dihydro-2H-chromen-4-ylcarbamoyl]hexahydropyrrolo[1,2-a]pyrazine-2-(1H)-carboxylate (843 mg) was dissolved in ethyl acetate (6 mL), 4M hydrogen chloride-ethyl acetate solution (12 mL) was added thereto, and the mixture was stirred at room temperature for 4 hr. The reaction mixture was concentrated under reduced pressure, and the precipitated solid was collected by filtration, and washed with ethyl acetate to give a colorless amorphous solid. This amorphous solid, (2S)-[(tert-butoxycarbonyl)amino](cyclohexyl)ethanoic acid (528 mg), 1-ethyl-3-(3-dimethylaminopropyl)carbodiimide hydrochloride (656 mg), 1-hydroxybenzotriazole (255 mg) and N,N-diisopropylethylamine (663 mg) were mixed in N,N-dimethylformamide (10 mL), and the mixture was stirred at room temperature for 18 hr. The mixture was diluted with ethyl acetate (70 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (615 mg) as a yellow oil. LC-MS: 611.4 (MH⁺).

### (iii) Production of tert-butyl[(1S)-2-({(1S)-1-cyclohexyl-2-[(3S,7R,8aR)-7-(cyclopropylmethoxy)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]methylcarbamate

tert-Butyl{(1S)-1-cyclohexyl-2-[(3S,7R,8aR)-7-(cyclopropylmethoxy)-3-[(4R)-3,4-dihydro-2H-chromen-4-ylcarbamoyl]hexahydropyrrolo[1,2-a]pyrazin-2(1H)-yl]-2-oxoethyl}carbamate (615 mg) was dissolved in ethyl acetate (4 mL), 4M hydrogen chloride-ethyl acetate solution (11 mL) was added thereto, and the mixture was stirred at room temperature for 2 hr. The reaction mixture was concentrated under reduced pressure, and the precipitated solid was collected by filtration, and washed with ethyl acetate to give a colorless amorphous solid. This amorphous solid, N-(tert-butoxycarbonyl)-N-methyl-L-alanine (210 mg), 1-ethyl-3-(3-dimethylaminopropyl)carbodiimide hydrochloride (328 mg), 1-hydroxybenzotriazole (127 mg) and N,N-diisopropylethylamine (332 mg) were mixed in N,N-dimethylformamide (5 mL), and the mixture was stirred at room temperature for 16 hr. The mixture was diluted with ethyl acetate (50 mL), and washed with water (30 mL), 10% aqueous sodium hydrogen carbonate solution (30 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (460 mg) as an oil. LC-MS: 696.5 (MH⁺).

### (iv) Production of (3S,7R,8aR)-2-{(2S)-2-cyclohexyl-2-[(N-methyl-L-alanyl)amino]acetyl}-7-(cyclopropylmethoxy)-N-[(4R)-3,4-dihydro-2H-chromen-4-yl]octahydropyrrolo[1,2-a]pyrazine-3-carboxamide

tert-Butyl[(1S)-2-({(13)-1-cyclohexyl-2-[(3S,7R,8aR)-7-(cyclopropylmethoxy)-3-[(4R)-3,4-dihydro-2H-chromen-4-ylcarbamoyl]hexahydropyrrolo[1,2-a]pyrazin-2(1H)-yl]-2-oxoethyl}amino)-1-methyl-2-oxoethyl]methylcarbamate (460 mg) was dissolved in ethyl acetate (6 mL), 4M hydrogen chloride-ethyl acetate solution (12 mL) was added thereto, and the mixture was stirred at room temperature for 2 hr. To the mixture was added water (20 mL), and the aqueous layer was separated. The aqueous layer was neutralized with 1M aqueous sodium hydroxide solution, and extracted with ethyl acetate (10 mL). The organic layer was washed with saturated brine (10 mL), dried over anhydrous magnesium sulfate, and concentrated under reduced pressure. The residue was dissolved in ethyl acetate (1 mL), heptane (3 mL) was added thereto, and the mixture was stirred at room temperature for 15 min. The precipitated solid was collected by filtration, and washed with ethyl acetate-heptane to give the title compound (126 mg) as a white solid. LC-MS: 596.4 (MH⁺).

¹H NMR (CDCl₃, 300 MHz): δ 0.13-0.21 (2H, m), 0.45-0.58 (2H, m), 0.74-1.33 (10H, m), 1.38-1.53 (3H, m), 1.66-1.83 (3H, m), 1.96-2.69 (9H, m), 2.90-3.26 (5H, m), 3.62-4.33 (5H, m), 4.48-4.86 (2H, m), 5.04-5.41 (2H, m), 6.52-6.98 (3H, m), 7.10-7.24 (2H, m), 7.49-7.82 (1H, m).

### Example 83

### Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-(2-ethoxyethoxy)octahydropyrrolo[1,2-a]pyrazine-3-carboxamide

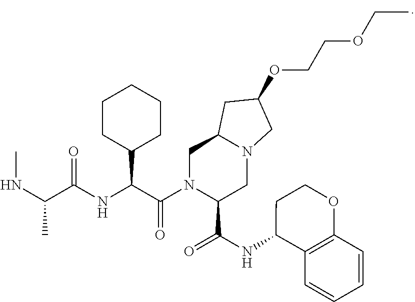

### (i) Production of tert-butyl(3S,7R,8aR)-3-[(4R)-3,4-dihydro-2H-chromen-4-ylcarbamoyl]-7-(2-ethoxyethoxy)hexahydropyrrolo[1,2-a]pyrazine-2(1H)-carboxylate

To a suspension of 2-tert-butyl 3-methyl(3S,7R,8aR)-7-hydroxyhexahydropyrrolo[1,2-a]pyrazine-2,3(1H)-dicarboxylate (1.05 g) in tetrahydrofuran (15 mL) was added sodium hydride (60%, 839 mg) under ice-cooling. The mixture was stirred at room temperature for 30 min, 1-bromo-2-ethoxyethane (3.74 g) was added thereto, and the mixture was stirred at 60° C. for 16 hr. The reaction mixture was allowed to cool to room temperature, diluted with methanol (10 mL) and ethyl acetate (30 mL), and concentrated under reduced pressure to give a yellow oil. This yellow oil, (4R)-3,4-dihydro-2H-chromen-4-amine hydrochloride (777 mg), 1-ethyl-3-(3-dimethylaminopropyl)carbodiimide hydrochloride (1.6 g), 1-hydroxybenzotriazole (450 mg) and N,N-diisopropylethylamine (1.07 g) were mixed in N,N-dimethylformamide (9 mL), and the mixture was stirred at room temperature for 15 hr. The mixture was diluted with ethyl acetate (70 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (1.10 g) as an oil. LC-MS: 490.3 (MH⁺).

### (ii) Production of tert-butyl{(1S)-1-cyclohexyl-2-[(3S,7R,8aR)-3-[(4R)-3,4-dihydro-2H-chromen-4-ylcarbamoyl]-7-(2-ethoxyethoxy)hexahydropyrrolo[1,2-a]pyrazin-2(1H)-yl]-2-oxoethyl}carbamate

tert-Butyl(3S,7R,8aR)-3-[(4R)-3,4-dihydro-2H-chromen-4-ylcarbamoyl]-7-(2-ethoxyethoxy)hexahydropyrrolo[1,2-a]pyrazine-2(1H)-carboxylate (1.10 g) was dissolved in methanol (5 mL), 4M hydrogen chloride-cyclopentyl methyl ether solution (20 mL) was added thereto, and the mixture was stirred at room temperature for 3 hr. The reaction mixture was concentrated under reduced pressure, and the precipitated solid was collected by filtration, and washed with ethyl acetate to give a colorless amorphous solid. This amorphous solid, (2S)-[(tert-butoxycarbonyl)amino](cyclohexyl)ethanoic acid (859 mg), 1-ethyl-3-(3-dimethylaminopropyl)carbodiimide hydrochloride (984 mg), 1-hydroxybenzotriazole (382 mg) and N,N-diisopropylethylamine (989 mg) were mixed in N,N-dimethylformamide (15 mL), and the mixture was stirred at room temperature for 17 hr. The mixture was diluted with ethyl acetate (50 mL), and washed with water (30 mL), 10% aqueous sodium hydrogen carbonate solution (30 mL) and saturated brine (20 mL). The organic layer was dried over anhydrous magnesium sulfate, and concentrated under reduced pressure. The residue was purified by silica gel column chromatography (ethyl acetate/hexane=0/100→100/0) to give the title compound (798 mg) as a yellow oil. LC-MS: 629.4 (MH⁺).

### (iii) Production of tert-butyl[(1S)-2-({(1S)-1-cyclohexyl-2-[(3S,7R,8aR)-3-[(4R)-3,4-dihydro-2H-chromen-4-ylcarbamoyl]-7-(2-ethoxyethoxy)hexahydropyrrolo[1,2-a]pyrazin-2(1H)-yl]-2-oxoethyl}amino)-1-methyl-2-oxoethyl]methylcarbamate

tert-Butyl{(1S)-1-cyclohexyl-2-[(3S,7R,8aR)-3-[(4R)-3,4-dihydro-2H-chromen-4-ylcarbamoyl]-7-(2-ethoxyethoxy)hexahydropyrrolo[1,2-a]pyrazin-2(1H)-yl]-2-oxoethyl}carbamate (798 mg) was dissolved in methanol (4 mL), 4M hydrogen chloride-cyclopentyl methyl ether solution (15 mL) was added thereto, and the mixture was stirred at room temperature for 4 hr. The reaction mixture was concentrated under reduced pressure, and the precipitated solid was collected by filtration, and washed with ethyl acetate to give a colorless amorphous solid. This amorphous solid, N-(tert-butoxycarbonyl)-N-methyl-L-alanine (327 mg), 1-ethyl-3-(3-dimethylaminopropyl)carbodiimide hydrochloride (514 mg), 1-hydroxybenzotriazole (199 mg) and N,N-diisopropylethylamine (520 mg) were mixed in N,N-dimethylformamide (8 mL), and the mixture was stirred at room temperature for 18 hr. The mixture was diluted with ethyl acetate (50 mL), and washed with water (50 mL), 10% aqueous sodium hydrogen carbonate solution (50 mL) and saturated brine (30 mL). The organic layer was dried over anhydrous magnesium sulfate, and concentrated under reduced pressure. The residue was purified by silica gel column chromatography (ethyl acetate/hexane=0/100→100/0) to give the title compound (841 mg) as an oil. LC-MS: 714.5 (MH⁺).

### (iv) Production of (3S,7R,8aR)-2-{(2S)-2-cyclohexyl-2-[(N-methyl-L-alanyl)amino]acetyl}-N-[(4R)-3,4-dihydro-2H-chromen-4-yl]-7-(2-ethoxyethoxy)octahydropyrrolo[1,2-a]pyrazine-3-carboxamide

tert-Butyl[(1S)-2-({(1S)-1-cyclohexyl-2-[(3S,7R,8aR)-3-[(4R)-3,4-dihydro-2H-chromen-4-ylcarbamoyl]-7-(2-ethoxyethoxy)hexahydropyrrolo[1,2-a]pyrazin-2(1H)-yl]-2-oxoethyl}amino)-1-methyl-2-oxoethyl]methylcarbamate (841 mg) was dissolved in methanol (4 mL), 4M hydrogen chloride-cyclopentyl methyl ether solution (15 mL) was added thereto, and the mixture was stirred at room temperature for 3 hr. To the mixture was added water (20 mL), and the aqueous layer was separated. The aqueous layer was neutralized with 1M aqueous sodium hydroxide solution, and extracted with ethyl acetate (10 mL). The organic layer was washed with saturated brine (10 mL), dried over anhydrous magnesium sulfate, and concentrated under reduced pressure. The residue was dissolved in diethyl ether (1 mL), pentane (3 mL) was added thereto, and the mixture was stirred at room temperature for 15 min. The precipitated solid was collected by filtration, and washed with diethyl ether-pentane to give the title compound (321 mg) as a white solid. LC-MS: 614.5 (MH⁺).

¹H NMR (CDCl₃, 300 MHz): δ 0.83-1.36 (12H, m), 1.45-2.29 (15H, m), 2.66-3.13 (2H, m), 3.19-3.29 (1H, m), 3.38-3.63 (7H, m), 3.92-5.18 (8H, m), 6.69-6.94 (2H, m), 7.05-7.32 (2H, m), 7.92-8.41 (2H, m).

### Example 84

### Production of (3S,7R,8aR)-2-[(2S)-2-cyclohexyl-2-(glycylamino)acetyl]-N-[(4R)-3,4-dihydro-2H-chromen-4-yl]-7-ethoxyoctahydropyrrolo[1,2-a]pyrazine-3-carboxamide

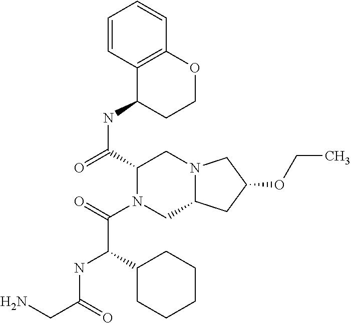

A mixture of 0.16 mol/L DMF solution (0.50 mL, 0.080 mmol) of (3S,7R,8aR)-2-[(2S)-2-amino-2-cyclohexylacetyl]-N-[(4R)-3,4-dihydro-2H-chromen-4-yl]-7-ethoxyoctahydropyrrolo[1,2-a]pyrazine-3-carboxamide and triethylamine (0.022 mL, 0.160 mmol), and 0.24 mol/L DMF solution (0.50 mL, 0.120 mmol) of N-[3-(dimethylamino)propyl]-N′-ethylcarbodiimide hydrochloride and 1H-benzotriazol-1-ol were added to N-(tert-butoxycarbonyl)glycine (14.0 mg, 0.080 mmol), and the mixture was stirred at room temperature for 3 hr. The reaction mixture was extracted with ethyl acetate (3 mL) and 2% aqueous sodium hydrogen carbonate solution (1 mL), and the organic layer was separated by top-phase separation filter tubes (manufactured by Wako Pure Chemical Industries, Ltd.). The solvent was evaporated under reduced pressure, the residue was dissolved in TFA (0.50 mL, 6.7 mmol), and the solution was stirred at room temperature for 1 hr, and concentrated. The residue was dissolved in acetonitrile/water (1/1) (1 mL), and the solution was purified by preparative HPLC to give the title compound.

yield: 17.4 mg.

The synthesis of the following Examples 85-101 was performed in the same manner as in the following Example 84, using the following apparatus and under the following conditions.

Apparatus: Waters MUX 4-ch LC-MS system

### Column: CAPCELL PAK C18 UG120, S-3 μm, 1.5×35 mm (Shiseido Co., Ltd.)

Mobile phase: SOLUTION A; 5 mM ammonium acetate-containing water, SOLUTION B; 5 mM ammonium acetate-containing acetonitrile
Gradient cycle: gradient: 0.00 min (SOLUTION A/SOLUTION B=100/0), 2.00 min (SOLUTION A/SOLUTION B=0/100), 3.00 min (SOLUTION A/SOLUTION B=0/100), 3.01 min (SOLUTION A/SOLUTION B=100/0), 3.30 min (SOLUTION A/SOLUTION B=100/0)
Injection volume: 2 μL,
Flow rate: 0.5 mL/min,
Detection method: UV 220 nm
Ionization method: electrospray method (ESI method)
Measurement mode: Full Scan (positive+negative ion)
measurement MS range: m/z=150-750

### Apparatus: Brucker AV400 (400 MHz)

Measurement solvent: DMSO-d₆
Internal standard: tetramethylsilane (TMS)
Instrument: Gilson, Inc. high throughput purification system

### Column: YMC CombiPrep, Pr° C18 RS S-5 μm, 20×50 mm (YMC)

Mobile phase: SOLUTION A; 10 mM ammonium bicarbonate-containing water, SOLUTION B; acetonitrile
Gradient cycle: 0.00 min (SOLUTION A/SOLUTION B=95/5), 2.00 min (SOLUTION A/SOLUTION B=95/5), 4.02 min (SOLUTION A/SOLUTION B=5/95) 6.40 min (SOLUTION A/SOLUTION B=5/95), 6.50 min (SOLUTION A/SOLUTION B=95/5), 8.00 min (SOLUTION A/SOLUTION B=95/5)
Injection volume: 500 μL,
Flow rate: 25 mL/min,
Detection method: UV 220 nm, 254 nm

TABLE 1

| Ex. | | | |
|---|---|---|---|
| No. | IUPAC name | structure | MS |
| 84 | (3S,7R,8aR)-2-[(2S)-2- cyclohexyl-2- (glycylamino)acetyl]-N-[(4R)- 3,4-dihydro-2H-chromen-4-yl]- 7-ethoxyoctahydropyrrolo[1,2- a]pyrazine-3-carboxamide | 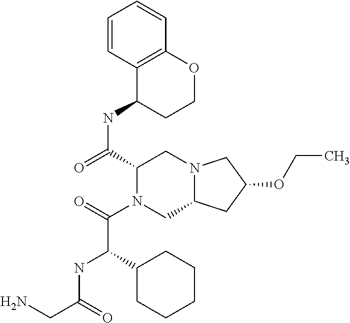 | 542.3 |
| 85 | (3S,7R,8aR)-2-{(2S)-2- cyclohexyl-2-[(N- methylglycyl)amino]acetyl}-N- [(4R)-3,4-dihydro-2H-chromen- 4-yl]-7- ethoxyoctahydropyrrolo[1,2- a]pyrazine-3-carboxamide | 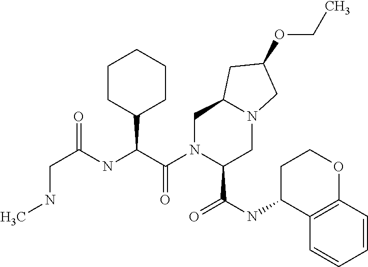 | 556.4 |
| 86 | (3S,7R,8aR)-2-[(2S)-2-(L- alanylamino)-2- cyclohexylacetyl]-N-[(4R)- 3,4-dihydro-2H-chromen-4-yl]- 7-ethoxyoctahydropyrrolo[1,2- a]pyrazine-3-carboxamide | 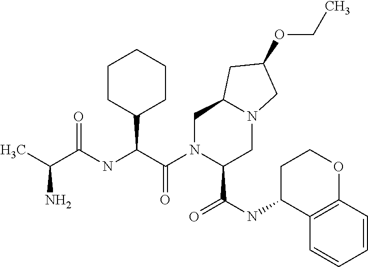 | 556.3 |
| 87 | (3S,7R,8aR)-2-{(2S)-2- cyclohexyl-2-[(N-ethyl-L- alanyl)amino]acetyl}-N-[(4R)- 3,4-dihydro-2H-chromen-4-yl]- 7-ethoxyoctahydropyrrolo[1,2- a]pyrazine-3-carboxamide | 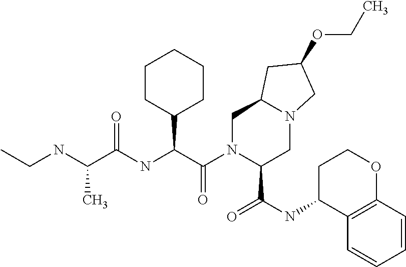 | 584.3 |
| 88 | (3S,7R,8aR)-2-[(2S)-2- cyclohexyl-2-{[N- (cyclopropylmethyl)-L- alanyl]amino}acetyl]-N-[(4R)- 3,4-dihydro-2H-chromen-4-yl]- 7-ethoxyoctahydropyrrolo[1,2- a]pyrazine-3-carboxamide | 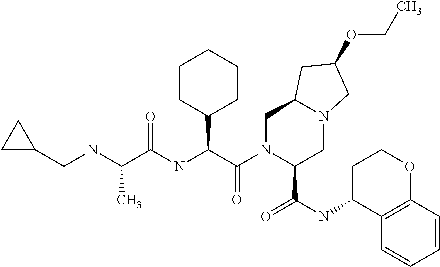 | 610.4 |
| 89 | (3S,7R,8aR)-2-{(2S)-2- cyclohexyl-2-[(3,3,3- trifluoroalanyl)amino]acetyl}- N-[(4R)-3,4-dihydro-2H- chromen-4-yl]-7- ethoxyoctahydropyrrolo[1,2- a]pyrazine-3-carboxamide | 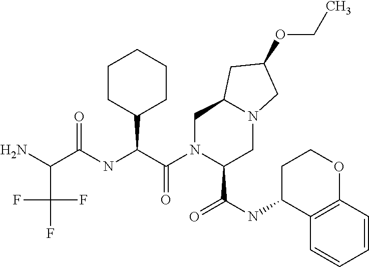 | 610.3 |
| 90 | (3S,7R,8aR)-2-{(2S)-2-[(2- amino-4,4,4- trifluorobutanoyl)amino]-2- cyclohexylacetyl}-N-[(4R)- 3,4-dihydro-2H-chromen-4-yl]- 7-ethoxyoctahydropyrrolo[1,2- a]pyrazine-3-carboxamide | 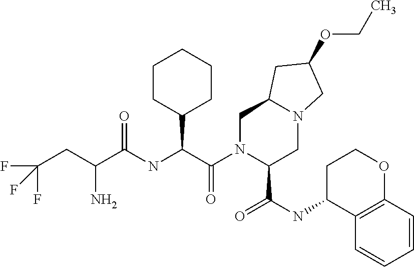 | 624.4 |
| 91 | (3S,7R,8aR)-2-[(2S)-2- cyclohexyl-2-{[(2S)-2- (methylamino)butanoyl]amino} acetyl]-N-[(4R)-3,4-dihydro- 2H-chromen-4-yl]-7- ethoxyoctahydropyrrolo[1,2- a]pyrazine-3-carboxamide | 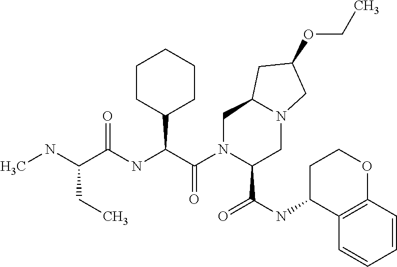 | 584.3 |
| 92 | (3S,7R,8aR)-2-{(2S)-2- cyclohexyl-2-[(3-cyclopropyl- L-alanyl)amino]acetyl}-N- [(4R)-3,4-dihydro-2H-chromen- 4-yl]-7- ethoxyoctahydropyrrolo[1,2- a]pyrazine-3-carboxamide | 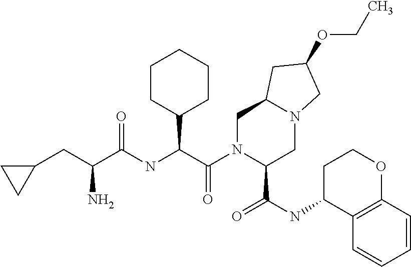 | 596.3 |
| 93 | (3S,7R,8aR)-2-[(2S)-2- cyclohexyl-2-(L- valylamino)acetyl]-N-[(4R)- 3,4-dihydro-2H-chromen-4-yl]- 7-ethoxyoctahydropyrrolo[1,2- a]pyrazine-3-carboxamide | 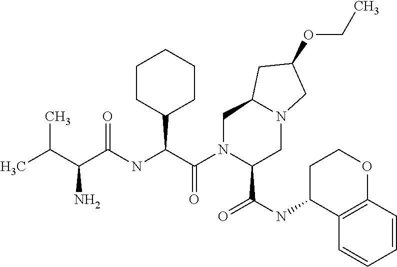 | 584.3 |
| 94 | (3S,7R,8aR)-2-[(2S)-2-{[(2S)- 2-amino-3- cyanopropanoyl]amino}-2- cyclohexylacetyl]-N-[(4R)- 3,4-dihydro-2H-chromen-4-yl]- 7-ethoxyoctahydropyrrolo[1,2- a]pyrazine-3-carboxamide | 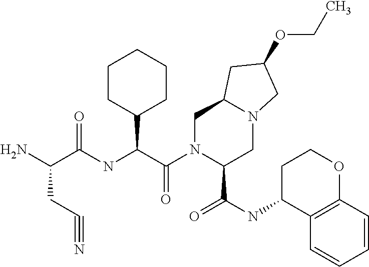 | 581.3 |
| 95 | (3S,7R,8aR)-2-[(2S)-2-{[(2S)- azetidin-2-ylcarbonyl]amino}- 2-cyclohexylacetyl]-N-[(4R)- 3,4-dihydro-2H-chromen-4-yl]- 7-ethoxyoctahydropyrrolo[1,2- a]pyrazine-3-carboxamide | 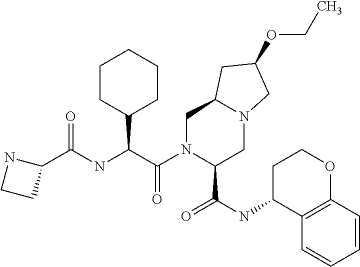 | 568.3 |
| 96 | (3S,7R,8aR)-2-[(2S)-2-{[(2R)- azetidin-2-ylcarbonyl]amino}- 2-cyclohexylacetyl]-N-[(4R)- 3,4-dihydro-2H-chromen-4-yl]- 7-ethoxyoctahydropyrrolo[1,2- a]pyrazine-3-carboxamide | 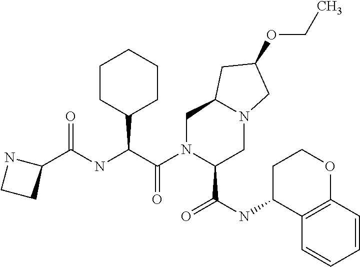 | 568.3 |
| 97 | (3S,7R,8aR)-2-[(2S)-2- cyclohexyl-2-(L- prolylamino)acetyl]-N-[(4R)- 3,4-dihydro-2H-chromen-4-yl]- 7-ethoxyoctahydropyrrolo[1,2- a]pyrazine-3-carboxamide | 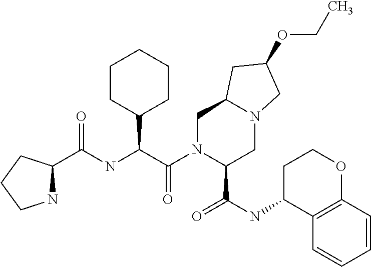 | 582.4 |
| 98 | (3S,7R,8aR)-2-{(2S)-2- cyclohexyl-2-[(N,N- dimethylglycyl)amino]acetyl}- N-[(4R)-3,4-dihydro-2H- chromen-4-yl]-7- ethoxyoctahydropyrrolo[1,2- a]pyrazine-3-carboxamide | 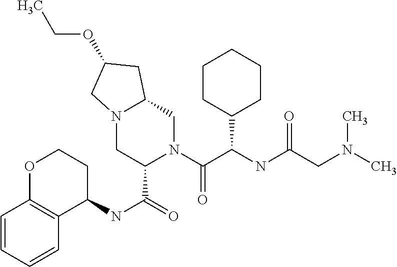 | 570.4 |
| 99 | (3S,7R,8aR)-2-[(2S)-2- cyclohexyl-2- (serylamino)acetyl]-N-[(4R)- 3,4-dihydro-2H-chromen-4-yl]- 7-ethoxyoctahydropyrrolo[1,2- a]pyrazine-3-carboxamide | 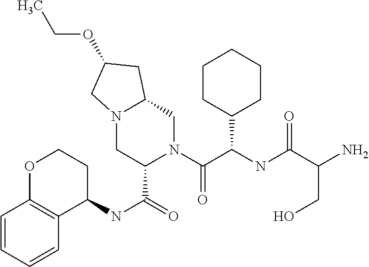 | 572.3 |
| 100 | (3S,7R,8aR)-2-[(2S)-2- cyclohexyl-2-(L- homoserylamino)acetyl]-N- [(4R)-3,4-dihydro-2H-chromen- 4-yl]-7- ethoxyoctahydropyrrolo[1,2- a]pyrazine-3-carboxamide | 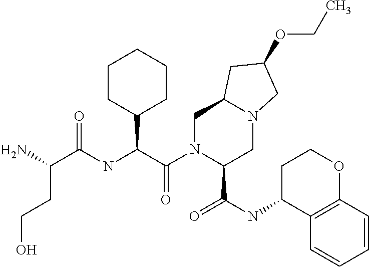 | 586.3 |
| 101 | (3S,7R,8aR)-2-{(2S)-2- cyclohexyl-2-[(N-methyl-L0 valyl)amino]acetyl}-N-[(4R)- 3,4-dihydro-2H-chromen-4-yl]- 7-ethoxyoctahydropyrrolo[1,2- a]pyrazine-3-carboxamide | 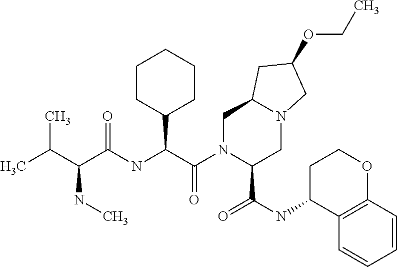 | 598.4 |

### Example 102

### Production of (3S,7R,8aR)-2-{(2S)-2-[(N-carbamimidoyl-L-alanyl)amino]-2-cyclohexylacetyl}-N-[(4R)-3,4-dihydro-2H-chromen-4-yl]-7-ethoxyoctahydropyrrolo[1,2-a]pyrazine-3-carboxamide tristrifluoroacetate

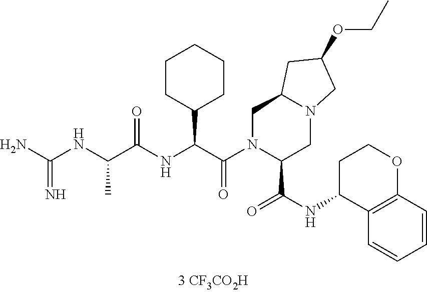

### (i) Production of tert-butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]carbamate

(3S,7R,8aR)-2-[(2S)-2-Amino-2-cyclohexylacetyl]-N-[(4R)-3,4-dihydro-2H-chromen-4-yl]-7-ethoxyoctahydropyrrolo[1,2-a]pyrazine-3-carboxamide (200 mg), (S)-2-(tert-butoxycarbonylamino)propanoic acid (94 mg), 1-ethyl-3-(3-dimethylaminopropyl)carbodiimide hydrochloride (119 mg), 1-hydroxybenzotriazole (55.8 mg) and N,N-diisopropylethylamine (0.108 mL) were mixed in N,N-dimethylformamide (2.00 mL), and the mixture was stirred at room temperature for 17 hr. To the reaction mixture was added saturated aqueous sodium hydrogen carbonate solution, and the mixture was extracted twice with ethyl acetate. The organic layers were combined, washed with saturated brine, dried over anhydrous magnesium sulfate, and concentrated under reduced pressure. The residue was purified by silica gel column chromatography (ethyl acetate/hexane=2/98→100/0) to give the title compound (165 mg) as an oil.

LC-MS: 656.2 (MH⁺).

### (ii) Production of (3S,7R,8aR)-2-[(2S)-2-(L-alanylamino)-2-cyclohexylacetyl]-N-[(4R)-3,4-dihydro-2H-chromen-4-yl]-7-ethoxyoctahydropyrrolo[1,2-a]pyrazine-3-carboxamide bistrifluoroacetate

tert-Butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]carbamate (165 mg) was dissolved in trifluoroacetic acid (2.00 mL), and the solution was stirred at room temperature for 3 hr. The reaction mixture was concentrated under reduced pressure, the residue was diluted with toluene and ethanol, and the mixture was concentrated under reduced pressure to give the title compound (367 mg) as a pale-yellow oil.

LC-MS: 556.1 (MH⁺).

### (iii) Production of tert-butyl[[(tert-butoxycarbonyl)amino]{[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]amino}methylidene]carbamate

(3S,7R,8aR)-2-[(2S)-2-(L-Alanylamino)-2-cyclohexylacetyl]-N-[(4R)-3,4-dihydro-2H-chromen-4-yl]-7-ethoxyoctahydropyrrolo[1,2-a]pyrazine-3-carboxamide bitrifluoroacetate (227 mg), tert-butyl[[(tert-butoxycarbonyl)amino]{[(trifluoromethyl)sulfonyl]amino}methylidene]carbamate (148 mg) and N,N-diisopropylethylamine (0.132 mL) were mixed in N,N-dimethylformamide (3.0 mL), and the mixture was stirred at room temperature for 16 hr. To the reaction mixture was added tert-butyl[[(tert-butoxycarbonyl)amino]{[(trifluoromethyl)sulfonyl]amino}methylidene]carbamate (148 mg), and the mixture was stirred at 70° C. for 5 hr. To the reaction mixture was added N,N-diisopropylethylamine (0.132 mL), and the mixture was stirred at 70° C. for 3 hr, and then at room temperature for 3 days. To the reaction mixture was added saturated aqueous sodium hydrogen carbonate solution, and the mixture was extracted twice with ethyl acetate. The organic layers were combined, washed with saturated brine, dried over anhydrous magnesium sulfate, and concentrated under reduced pressure. The residue was purified by silica gel column chromatography (ethyl acetate/hexane=2/98→100/0) to give the title compound (12.0 mg) as an oil.

LC-MS: 798.3 (MH⁺).

### (iv) Production of (3S,7R,8aR)-2-{(2S)-2-[(N-carbamimidoyl-L-alanyl)amino]-2-cyclohexylacetyl}-N-[(4R)-3,4-dihydro-2H-chromen-4-yl]-7-ethoxyoctahydropyrrolo[1,2-a]pyrazine-3-carboxamide tristrifluoroacetate

tert-Butyl[[(tert-butoxycarbonyl)amino]{[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]amino}methylidene]carbamate (12.0 ng) was dissolved in trifluoroacetic acid (1.00 mL), and the solution was stirred at room temperature for 3 hr. The reaction mixture was concentrated under reduced pressure, the residue was diluted with acetonitrile, and the mixture was concentrated under reduced pressure to give the title compound (10.5 mg) as a brown oil.

LC-MS: 598.1 (MH⁺).

¹H NMR (CDCl₃, 300 MHz): δ 0.71-2.80 (21H, m), 3.14-3.72 (6H, m), 3.78-4.55 (7H, m), 4.60-5.25 (3H, m), 6.07-7.22 (9H, m), 8.13 (1H, brs), 8.34 (1H, brs), 9.17 (1H, brs), 11.90 (1H, brs).

### Example 103

### Production of (3S,7R,8aR)-2-{(2S)-2-cyclohexyl-2-[(N-methyl-L-seryl)amino]acetyl}-N-[(4R)-3,4-dihydro-2H-chromen-4-yl]-7-ethoxyoctahydropyrrolo[1,2-a]pyrazine-3-carboxamide bistrifluoroacetate

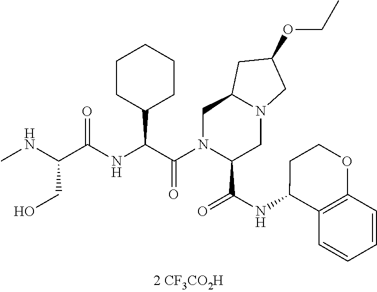

### (i) Production of tert-butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(hydroxymethyl)-2-oxoethyl]methylcarbamate

(3S,7R,8aR)-2-[(2S)-2-Amino-2-cyclohexylacetyl]-N-[(4R)-3,4-dihydro-2H-chromen-4-yl]-7-ethoxyoctahydropyrrolo[1,2-a]pyrazine-3-carboxamide (100 mg), (S)-2-(tert-butoxycarbonyl(methyl)amino)-3-hydroxypropanoic acid (54.3 mg) and 4-(4,6-dimethoxy-1,3,5-triazin-2-yl)-4-methylmorpholin-4-ium chloride (68.5 mg) were mixed in a mixed solvent of tetrahydrofuran (2.00 mL) and 2-propanol (2.00 mL), and the mixture was stirred at room temperature for 17 hr. To the reaction mixture was added saturated aqueous sodium hydrogen carbonate solution, and the mixture was extracted twice with as ethyl acetate. The organic layers were combined, washed with saturated brine, dried over anhydrous magnesium sulfate, and concentrated under reduced pressure. The residue was purified by silica gel column chromatography (methanol/ethyl acetate=0/100→10/90) to give the title compound (91.0 mg) as an oil.

LC-MS: 686.3 (MH⁺).

### (ii) Production of (3S,7R,8aR)-2-{(2S)-2-cyclohexyl-2-[(N-methyl-L-seryl)amino]acetyl}-N-[(4R)-3,4-dihydro-2H-chromen-4-yl]-7-ethoxyoctahydropyrrolo[1,2-a]pyrazine-3-carboxamide bistrifluoroacetate

tert-Butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(hydroxymethyl)-2-oxoethyl]methylcarbamate (91.0 mg) was dissolved in trifluoroacetic acid (1.00 mL), and the solution was stirred at room temperature for 6 hr. The reaction mixture was concentrated under reduced pressure, saturated aqueous sodium hydrogen carbonate solution was added thereto, and the mixture was extracted twice with ethyl acetate. The organic layers were combined, washed with saturated brine, dried over anhydrous magnesium sulfate, and concentrated under reduced pressure. The residue was dissolved in acetonitrile, and the solution was purified by preparative HPLC to give the title compound (55.0 mg).

LC-MS: 586.3 (MH⁺).

¹H NMR (300 MHz, CDCl₃): δ 0.73-2.80 (22H, m), 2.91-5.41 (17H, m), 6.78 (2H, d, J=7.7 Hz), 7.08 (2H, brs), 7.30-11.88 (5H, m).

### Example 104

### Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-hydroxyoctahydropyrrolo[1,2-a]pyrazine-3-carboxamide dihydrochloride

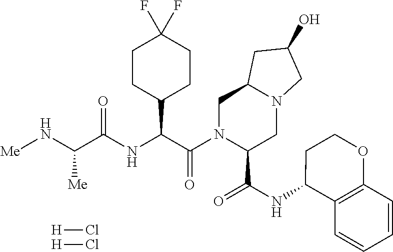

### (i) Production of tert-butyl(3S,7R,8aR)-3-[(4R)-3,4-dihydro-2H-chromen-4-ylcarbamoyl]-7-hydroxyhexahydropyrrolo[1,2-a]pyrazine-2(1H)-carboxylate

To a solution of 2-tert-butyl 3-methyl(3S,7R,8aR)-7-hydroxyhexahydropyrrolo[1,2-a]pyrazine-2,3(1H)-dicarboxylate (2.0 g) in tetrahydrofuran (30 mL)/water (10 mL) was added lithium hydroxide monohydrate (418 mg) at room temperature, and the reaction mixture was stirred at 50° C. for 18 hr. The reaction mixture was allowed to cool to room temperature, neutralized with 1M hydrochloric acid (9 mL), and concentrated under reduced pressure. The obtained residue was dissolved in N,N-dimethylformamide (20 mL), 1-hydroxybenzotriazole (1.08 g), N,N-diisopropylethylamine (2.32 mL) and 1-ethyl-3-(3-dimethylaminopropyl)carbodiimide (3.53 mL) were added thereto, and the mixture was stirred at room temperature for 10 min. (4R)-3,4-Dihydro-2H-chromen-4-amine hydrochloride (1.48 g) was added thereto, and the reaction mixture was stirred at room temperature for 3 days. The reaction mixture was diluted with ethyl acetate (200 mL) and saturated aqueous sodium hydrogen carbonate solution (100 mL), and the organic layer was separated. The organic layer was washed with water (50 mL) and saturated brine (50 mL), dried over anhydrous magnesium sulfate, and filtered. The filtrate was concentrated under reduced pressure, and the residue was washed with hexane (50 mL)/diethyl ether (50 mL) to give the title compound (2.15 g) as a colorless amorphous powder. LC-MS: 418.2 (MH⁺)

¹H NMR (DMSO-d₆, 300 MHz): δ 1.08-1.30 (1H, m), 1.31-1.50 (9H, m), 1.60-2.32 (6H, m), 2.62-3.19 (2H, m), 3.21-3.48 (1H, m), 3.72-3.95 (1H, m), 4.03-4.32 (3H, m), 4.32-4.59 (1H, m), 4.69-4.88 (1H, m), 4.90-5.19 (1H, m), 6.59-7.00 (2H, m), 7.04-7.27 (2H, m), 8.06-8.55 (1H, m).

### (ii) Production of (3S,7R,8aR)—N-[(4R)-3,4-dihydro-2H-chromen-4-yl]-7-hydroxyoctahydropyrrolo[1,2-a]pyrazine-3-carboxamide dihydrochloride

tert-Butyl(3S,7R,8aR)-3-[(4R)-3,4-dihydro-2H-chromen-4-ylcarbamoyl]-7-hydroxyhexahydropyrrolo[1,2-a]pyrazine-2(1H)-carboxylate (1.00 g) was dissolved in 4M hydrogen chloride-ethyl acetate solution (10 mL), and the solution was stirred at room temperature for 15 hr. The solvent was evaporated under reduced pressure to give the title compound (840 mg) as a colorless amorphous powder. LC-MS: 318.2 (MH⁺)

### (iii) Production of benzyl{(1S)-1-(4,4-difluorocyclohexyl)-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}carbamate

To a solution of 1-hydroxybenzotriazole (340 mg), N,N-diisopropylethylamine (1.13 mL) and 1-ethyl-3-(3-dimethylaminopropyl)carbodiimide (1.14 mL) in N,N-dimethylformamide (10 mL) was added (2S)-{[(benzyloxy)carbonyl]amino}(4,4-difluorocyclohexyl)ethanoic acid (775 mg), and the mixture was stirred at room temperature for 5 min. (3S,7R,8aR)—N-[(4R)-3,4-Dihydro-2H-chromen-4-yl]-7-hydroxyoctahydropyrrolo[1,2-a]pyrazine-3-carboxamide dihydrochloride (840 mg) was added thereto, and the reaction mixture was stirred at room temperature for 15 hr. Ethyl acetate (200 mL)/water (50 mL) was added thereto, and the organic layer was separated. The organic layer was washed with water (100 mL) and saturated brine (50 mL), dried over anhydrous magnesium sulfate, and filtered. The filtrate was concentrated under reduced pressure, and the residue was purified by silica gel column chromatography (hexane/ethyl acetate=95/5→0/100→ethyl acetate/methanol=90/10). The object fractions were collected, the solvent was evaporated under reduced pressure, and the residue was dried in vacuum to give the title compound (575 mg) as white crystals. LC-MS: 627.1 (MH⁺)

¹H NMR (DMSO-d₆, 300 MHz): δ 1.08-1.49 (3H, m), 1.50-2.46 (13H, m), 2.54-3.06 (2H, m), 3.06-3.74 (2H, m), 3.80-4.30 (3H, m), 4.30-4.54 (1H, m), 4.63-5.27 (5H, m), 6.69-6.80 (1H, m), 6.80-6.95 (1H, m), 7.00-7.47 (7H, m), 7.49-8.52 (2H, m).

### (iv) Production of (3S,7R,8aR)-2-[(2S)-2-amino-2-(4,4-difluorocyclohexyl)acetyl]-N-[(4R)-3,4-dihydro-2H-chromen-4-yl]-7-hydroxyoctahydropyrrolo[1,2-a]pyrazine-3-carboxamide

Benzyl{(1S)-1-(4,4-difluorocyclohexyl)-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}carbamate (500 mg) was dissolved in hydrogen chloride-methanol solution (10 mL), 20% palladium hydroxide-carbon (100 mg, 20 wt %) was added thereto, and the mixture was stirred at room temperature for 18 hr under a hydrogen atmosphere (1 atm). After nitrogen substitution the insoluble material was filtered off through celite, and washed with methanol. The filtrate was concentrated under reduced pressure to give a colorless oil (383 mg). LC-MS: 493.1 (MH⁺)

### (v) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}amino)-1-methyl-2-oxoethyl]methylcarbamate

(3S,7R,8aR)-2-[(2S)-2-Amino-2-(4,4-difluorocyclohexyl)acetyl]-N-[(4R)-3,4-dihydro-2H-chromen-4-yl]-7-hydroxyoctahydropyrrolo[1,2-a]pyrazine-3-carboxamide (383 mg), N-(tert-butoxycarbonyl)-N-methyl-L-alanine (190 mg), 1-hydroxybenzotriazole (116 mg) and N,N-diisopropylethylamine (0.272 mL) were dissolved in N,N-dimethylformamide (5 mL), 1-ethyl-3-(3-dimethylaminopropyl)carbodiimide (0.413 mL) was added thereto, and the mixture was stirred at room temperature for 3 hr. The reaction mixture was diluted with ethyl acetate (100 mL)/water (25 mL), and the organic layer was separated. The organic layer was washed with saturated aqueous sodium hydrogen carbonate (25 mL) and saturated brine (25 mL), dried over anhydrous magnesium sulfate, and filtered. The filtrate was concentrated under reduced pressure, and the residue was purified by silica gel column chromatography (hexane/ethyl acetate=95/5→0/100). The object fractions were collected, the solvent was evaporated under reduced pressure, and the residue was dried in vacuum to, give the title compound (144 mg) as a white amorphous solid.

LC-MS: 678.4 (MH⁺)

¹H NMR (DMSO-d₆, 300 MHz): δ 1.08-1.30 (7H, m), 1.39 (9H, brs), 1.52-2.29 (15H, m), 2.57-2.89 (2H, m), 3.08-3.97 (2H, m), 4.09-4.29 (3H, m), 4.30-5.26 (5H, m), 6.62-6.95 (2H, m), 7.04-7.34 (2H, m), 7.54-8.47 (2H, m).

### (vi) Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-hydroxyoctahydropyrrolo[1,2-a]pyrazine-3-carboxamide dihydrochloride

To a solution of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-hydroxyhexahydropyrrolo[1,2-a]pyrazin-2(1H)-yl]-2-oxoethyl}amino)-1-methyl-2-oxoethyl]methylcarbamate (130 mg) in methanol (1 mL) was added 4N hydrogen chloride-cyclopentyl methyl ether solution (5 mL), and the mixture was stirred at room temperature for 2 hr. The reaction mixture was concentrated under reduced pressure, and the residue was washed with diethyl ether (20 mL). The resulting solid was dried under reduced pressure to give the title compound (93 mg) as a colorless amorphous powder.

LC-MS: 578.3 (MH⁺)

¹H NMR (DMSO-d₆, 300 MHz): δ 1.23-1.53 (5H, m), 1.54-2.38 (10H, m), 2.41-2.49 (3H, m), 3.02-4.30 (10H, m), 4.32-4.77 (3H, m), 4.78-5.11 (2H, m), 5.78 (1H, brs), 6.65-6.98 (2H, m), 7.04-7.58 (2H, m), 8.28-9.69 (4H, m), 11.71-12.92 (1H, m).

### Example 105

### Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

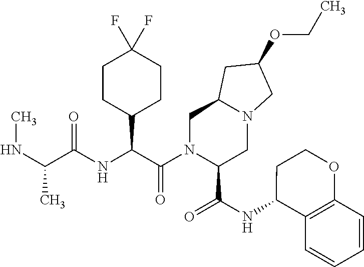

### (i) Production of tert-butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate

To a solution of 2-tert-butyl 3-methyl(3S,7R,8aR)-7-ethoxyhexahydropyrrolo[1,2-a]pyrazine-2,3(1H)-dicarboxylate (183 g) in methanol (1800 mL)/water (900 mL) was added lithium hydroxide monohydrate (37.4 g) at room temperature, and the reaction mixture was stirred at 50° C. for 3 hr. The reaction mixture was cooled to 10° C., neutralized with 0.5M hydrochloric acid (1200 mL), and concentrated under reduced pressure. The residue was subjected twice to azeotropic distillation with toluene, and the obtained residue was dissolved in N,N-dimethylformamide (1800 mL). 1-Hydroxybenzotriazole (82.9 g) and N,N-diisopropylethylamine (320 mL) were added thereto, and then 1-ethyl-3-(3-dimethylaminopropyl)carbodiimide hydrochloride (139 g) and (4R)-3,4-dihydro-2H-chromen-4-amine hydrochloride (114 g) were added thereto. The obtained reaction mixture was stirred at room temperature for 16 hr, and the solvent was evaporated under reduced pressure. The obtained residue was diluted with ethyl acetate (3600 mL), water (900 mL) and saturated aqueous sodium hydrogen carbonate (900 mL), and the organic layer was separated. The obtained aqueous layer was extracted with ethyl acetate (1800 mL), and the obtained organic layer was combined with the above-mentioned organic layer. The combined organic layer was washed with saturated brine (900 mL), dried over anhydrous sodium sulfate, and filtered. The filtrate was purified by silica gel column chromatography (ethyl acetate). The object fractions were collected, and the solvent was evaporated under reduced pressure. The obtained residue was suspended in diisopropyl ether (900 mL), and the suspension was filtered. The obtained crystals were washed with diisopropyl ether, and dried in vacuum to give the title compound (210 g) as a white powder. The washing solution was concentrated under reduced pressure, the residue was suspended in diisopropyl ether (100 mL), and the suspension was filtered. The obtained crystals were washed with diisopropyl ether, and dried in vacuum to give the title compound (10.4 g) as a white powder.

LC-MS: 446.3 (MH⁺)

¹H NMR (DMSO-d₆, 300 MHz): δ 1.09 (3H, t, J=7.1 Hz), 1.15-1.35 (1H, m), 1.35-1.45 (9H, m), 1.65-2.25 (6H, m), 2.87-3.08 (2H, m), 3.33 (2H, q, J=7.1 Hz), 3.30-3.45 (1H, m), 3.80-4.00 (2H, m), 4.15-4.35 (2H, m), 4.37-4.53 (1H, m), 5.02-5.14 (1H, m), 6.73-6.89 (2H, m), 7.10-7.19 (2H, m), 8.20-8.40 (1H, m).

### (ii) Production of benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate

tert-Butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate (150 g) was dissolved in a mixed solvent of 2-propanol (360 mL)/tetrahydrofuran (840 mL). The obtained solution was heated to 60° C., and 4M hydrogen chloride-cyclopropyl methyl ether solution (1050 mL) was added thereto at 60° C., and the mixture was stirred at 60° C. for 2 hr. Tetrahydrofuran (750 mL) was added thereto, and the mixture was stirred again at room temperature for 1 hr. The precipitate was collected by filtration, washed with a mixed solvent of tetrahydrofuran/diisopropyl ether (=1/1), and dried in vacuum to give (3S,7R,8aR)—N-[(4R)-3,4-dihydro-2H-chromen-4-yl]-7-hydroxyoctahydropyrrolo[1,2-a]pyrazine-3-carboxamide dihydrochloride (146.9 g). The same reaction was repeated using tert-butyl(3S,7R,8aR)-3-[(4R)-3,4-dihydro-2H-chromen-4-ylcarbamoyl]-7-ethoxyhexahydropyrrolo[1,2-a]pyrazine-2(1H)-carboxylate (150 g), and the obtained (3S,7R,8aR)—N-[(4R)-3,4-dihydro-2H-chromen-4-yl]-7-hydroxyoctahydropyrrolo[1,2-a]pyrazine-3-carboxamide dihydrochloride (141.5 g) was combined with the compound obtained above. The combined compound was dissolved in N,N-dimethylformamide (3000 mL). To the obtained solution were added (2S)-{[(benzyloxy)carbonyl]amino}(4,4-difluorocyclohexyl)ethanoic acid (231 g) and 1-hydroxybenzotriazole (100 g) at 5-10° C., and then the triethylamine (289 mL) and 1-ethyl-3-(3-dimethylaminopropyl)carbodiimide hydrochloride (168 g) were added thereto, and the mixture was stirred at room temperature for 14 hr. The reaction mixture was concentrated under reduced pressure, and the obtained residue was dissolved in ethyl acetate (3000 mL)/tetrahydrofuran (1500 mL), water (1500 mL) and saturated aqueous sodium hydrogen carbonate (1500 mL). The organic layer was separated from the obtained solution. The obtained aqueous layer was extracted with a mixed solvent of ethyl acetate/tetrahydrofuran (=2/1) (1500 mL), and combined with the above-mentioned organic layer. The combined organic layer was, washed twice with saturated brine (1500 mL), dried over anhydrous sodium sulfate, and filtered. The filtrate was purified by silica gel column chromatography (ethyl acetate), the object fractions were collected, and the solvent was evaporated under reduced pressure. The obtained residue was suspended in diisopropyl ether (1500 mL), and the suspension was stirred at room temperature for 14 hr, and filtered. The obtained crystals were washed with diisopropyl ether, and dried in vacuum to give the title compound (422 g) as a colorless powder.

LC-MS: 655.4 (MH⁺)

¹H NMR (DMSO-d₆, 300 MHz): δ 1.10 (3H, t, J=7.1 Hz), 1.15-1.42 (2H, m), 1.50-2.24 (13H, m), 2.82-3.73 (5H, m), 3.80-4.27 (3H, m), 4.28-5.18 (6H, m), 6.67-6.92 (2H, m), 7.06-7.18 (2H, m), 7.19-7.79 (7H, m), 8.08-8.24 (1H, m).

### (iii) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methyl carbamate

Benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (85 g) was dissolved in methanol (850 mL), 2M hydrochloric acid-methanol (136 mL) and 10% palladium-carbon (8.5 g, 50 wt %) were added thereto, and the mixture was stirred at room temperature for 2 hr under a hydrogen atmosphere. The above-mentioned reaction was repeated two more times using benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate. After nitrogen substitution, the obtained solutions were combined, and the insoluble material was filtered off, and washed with methanol. The obtained filtrate was concentrated under reduced pressure, the residue was suspended in toluene (200 mL), and the solvent was evaporated to give a yellow oil (223 g). The obtained yellow oil (199 g) was dissolved in N,N-dimethylformamide (2550 mL), the obtained solution was cooled to 5-10° C., N,N-diisopropylethylamine (204 mL), 1-hydroxybenzotriazole (57 g), N-(2,2-dimethylpropanoyl)-N-methyl-L-alanine (85.5 g) and 1-ethyl-3-(3-dimethylaminopropyl)carbodiimide hydrochloride (95 g) were added thereto. The obtained solution was stirred at room temperature for 12 hr. The solvent was evaporated under reduced pressure, the obtained residue was dissolved in ethyl acetate (2550 mL), tetrahydrofuran (1000 mL), water (1275 mL) and saturated aqueous sodium hydrogen carbonate (1275 mL), and the organic layer was separated. The obtained aqueous layer was extracted with a mixed solvent (1250 mL) of ethyl acetate/tetrahydrofuran (=2/1), and combined with the organic layer separated above. The combined organic layer was washed twice with saturated brine (1500 mL), dried over anhydrous sodium sulfate, and filtered. The filtrate was purified by silica gel column chromatography (ethyl acetate), the object fractions were collected by filtration, and the solvent was evaporated under reduced pressure to give the title compound (251 g). The same reaction was repeated using benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (85 g×4). The obtained title compound (354 g) was combined with the title compound obtained above, and the combined compound was dissolved in tetrahydrofuran (2900 mL) heated to 55-60° C. To the obtained solution was added dropwise heptane (2050 mL) at 55-60° C. The obtained suspension was stirred at 55-60° C. for 30 min. Additional heptane (900 mL) was added dropwise thereto, and the obtained suspension was stirred at 55-60° C. for 15 min, and then at room temperature for 30 min. The obtained precipitate was collected by filtration, washed with a mixed solvent of tetrahydrofuran/heptane (=1/1), and dried in vacuum to give the title compound (549 g) as a colorless powder.

LC-MS: 706.4 (MH⁺)

¹H NMR (DMSO-d₆, 300 MHz): δ 1.10 (3H, t, J=7.1 Hz), 1.10-1.36 (15H, m), 1.55-2.24 (13H, m), 2.57-3.68 (8H, m), 3.86-5.17 (8H, m), 6.70-6.90 (2H, m), 7.07-7.29 (2H, m), 7.65-8.32 (2H, m).

### (iv) Production of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

To a solution of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (255 g) in tetrahydrofuran (765 mL) was added concentrated hydrochloric acid (460 mL) at 5-10° C. The obtained solution was stirred at room temperature for 5 hr, cooled (5-10° C.), and neutralized with 4M aqueous sodium hydroxide solution. The obtained solution was extracted successively with ethyl acetate (770 mL) and a mixed solvent (800 mL) of ethyl acetate/tetrahydrofuran (=2/1), and the extract was washed with saturated brine (750 mL), dried over anhydrous sodium sulfate, and filtered. The filtrate was concentrated under reduced pressure, and the obtained residue was suspended in diisopropyl ether (750 mL). The obtained suspension was stirred at room temperature for 30 min, and filtered. The obtained crystals were washed with diisopropyl ether, and dried in vacuum to give crude crystals (220 g) of the title compound as a white powder. The above-mentioned reaction was repeated using tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (250 g, 30 g). The obtained crude crystals (216.24 g) were combined with the crude crystals obtained above. The combined crystals were dissolved in 2-butanone (1380 mL) heated to 55-60° C., and filtered. The obtained insoluble material was washed with 2-butanone (230 mL). To the washing solution heated to 55-60° C. was added dropwise heptane (1520 mL), and the mixture was stirred at 55-60° C. for 1 hr. Additional heptane (1470 mL) was added dropwise thereto, and the mixture was stirred at 55-60° C. for 20 min, and then at room temperature for 1 hr. The precipitate was collected by filtration, and the obtained crystals were washed with a mixed solvent of 2-butanone/heptane(=1/4), and dried in vacuum to give the title compound (402 g) as colorless crystals.

LC-MS: 606.4 (MH⁺)

¹H NMR (DMSO-d₆, 300 MHz): δ 1.01-1.15 (6H, m), 1.15-1.38 (3H, m), 1.54-2.29 (16H, m), 2.63-3.64 (7H, m), 3.88-4.48 (4H, m), 4.59-5.15 (3H, m), 6.70-6.80 (1H, m), 6.80-6.92 (1H, m), 7.07-7.30 (2H, m), 7.88-8.12 (1H, m), 8.12-8.33 (1H, m).

### (v) Recrystallization of (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide

(3S,7R,8aR)-2-{(2S)-2-(4,4-Difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide (5.00 g) was dissolved in 2-butanone (25 mL) at 77° C. (inside temperature was 60° C.). Heptane (40 mL) was added dropwise to the mixture at 77° C. (inside temperature was above 60° C.). The mixture was stirred at 77° C. for 1 hour (inside temperature was 65° C.). Heptane (60 mL) was added dropwise at 77° C. (inside temperature was above 60° C.) and the suspension was cooled to 5° C. slowly. The precipitated crystals were collected by filtration, washed with heptane (30 mL) and dried in vacuo (70° C. for 1 hour) to give the title compound (white crystals, 4.56 g).

LC-MS: 606.4 (MH⁺).

¹H NMR (300 MHz, DMSO-d_{6): δ} 1.01-1.15 (6H, m), 1.15-1.38 (3H, m), 1.54-2.29 (16H, m), 2.63-3.64 (7H, m), 3.88-4.48 (4H, m), 4.59-5.15 (3H, m), 6.70-6.80 (1H, 6.80-6.92 (1H, m), 7.07-7.30 (2H, m), 7.88-8.12 (1H, m), 8.12-8.33 (1H, m).

mp 178.6° C.

### Example 106

### Production of (3S,7R,8aR)-2-{(2S)-2-(4,4-difluorocyclohexyl)-2-[(N-methyl-D-alanyl)amino]acetyl}-N-[(4R)-3,4-dihydro-2H-chromen-4-yl]-7-ethoxyoctahydropyrrolo[1,2-a]pyrazine-3-carboxamide

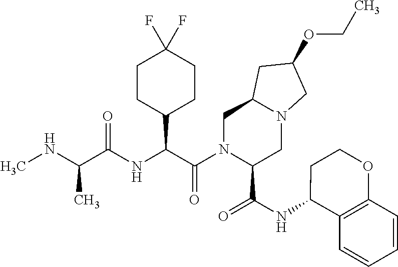

### (i) Production of tert-butyl[(1R)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate

A mixture of benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (300 mg) and palladium hydroxide (72 mg) in 10% hydrogen chloride-methanol solution (12 mL) was stirred under hydrogen atmosphere at room temperature for 3 hours. The mixture was filtered through membrane filter and the filtrate was evaporated in vacuo to give (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride (255 mg) as a pale yellow solid. The obtained solid (255 mg), N-(tert-butoxycarbonyl)-N-methyl-D-alanine (102 mg) and 1-hydroxybenzotriazol (74 mg) were dissolved in N,N-dimethylformamide (5.0 mL). To the solution were added successively 1-ethyl-3-(3-dimethylaminopropyl)carbodiimide hydrochloride (263 mg) and N,N-diisopropylethylamine (0.399 mL) at 0° C., and the mixture was stirred at room temperature for 17 hours. The mixture was diluted with water and extracted twice with ethyl acetate. The combined extracts were washed with saturated sodium hydrogen carbonate solution and brine. The aqueous layer was extracted twice with ethyl acetate. The combined organic layers were dried over anhydrous magnesium sulfate and concentrated in vacuo. The residue was purified by silica gel chromatography (eluent; ethyl acetate/hexane=50/50→100/0) to give the title compound (313 mg) as pale yellow oil.

LC-MS: 706.4 (MH⁺).

### (ii) Production of (3S,7R,8aR)-2-{(2S)-2-(4,4-difluorocyclohexyl)-2-[(N-methyl-D-alanyl)amino]acetyl}-N-[(4R)-3,4-dihydro-2H-chromen-4-yl]-7-ethoxyoctahydropyrrolo[1,2-a]pyrazine-3-carboxamide

A solution of tert-butyl[(1R)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-methyl-2-oxoethyl]methylcarbamate (300 mg) in 4M hydrogen chloride-ethyl acetate solution (6.0 mL) was stirred at room temperature for 30 minutes. The mixture was concentrated in vacuo. The residue was treated with saturated sodium hydrogen carbonate solution and extracted four times with a mixed solvent of ethyl acetate-tetrahydrofuran (1:1). The extracts were washed with brine, dried over anhydrous magnesium sulfate and concentrated in vacuo. The residue was purified by silica gel chromatography (eluent; methanol/ethyl acetate=5/95-30/70) and recrystallized from ethyl acetate-hexane to give the title compound (137 mg) as a white solid.

LC-MS: 606.4 (MH⁺).

¹H NMR (300 MHz, DMSO-d₆): δ 1.00-1.15 (6H, m), 1.16-1.40 (3H, m), 1.57-2.30 (17H, m), 2.91-3.05 (2H, m), 3.24 (1H, t, J=11.5 Hz), 3.33-3.41 (2H, m), 3.48 (1H, d, J=10.8 Hz), 3.89-4.08 (2H, m), 4.12-4.27 (2H, m), 4.71-4.87 (1H, m), 4.93-5.15 (2H, m), 6.70-6.80 (1H, m), 6.80-6.93 (1H, m), 7.06-7.30 (2H, m), 8.02-8.23 (2H, m).

### Example 107

### Production of (3S,7R,8aR)-2-{(2S)-2-cyclohexyl-2-[(N-methyl-L-alanyl)amino]acetyl}-7-ethoxy-N-(4-phenyl-1,2,3-thiadiazol-5-yl)octahydropyrrolo[1,2-a]pyrazine-3-carboxamide

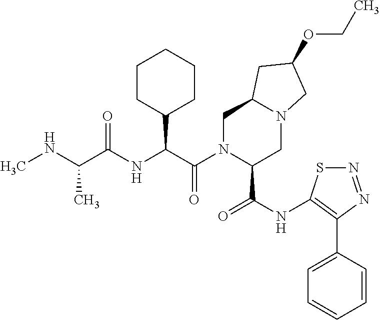

### (i) Production of tert-butyl(3S,7R,8aR)-7-ethoxy-3-[(4-phenyl-1,2,3-thiadiazol-5-yl)carbamoyl]hexahydropyrrolo[1,2-a]pyrazine-2(1H)-carboxylate

A mixture of 2-tert-butyl 3-methyl(3S,7R,8aR)-7-ethoxyhexahydropyrrolo[1,2-a]pyrazine-2,3(1H)-dicarboxylate (513 mg) and lithium hydroxide monohydrate (105 mg) in tetrahydrofuran (12.5 mL)/water (2.5 mL) was stirred at 50° C. for 5 hours. The mixture was neutralized with 1N hydrochloric acid, concentrated in vacuo and the residue was subjected three times to azeotropic distillation with toluene. The residue was dissolved in N,N-dimethylformamide (7.5 mL), and to the solution were added 4-phenyl-1,2,3-thiadiazol-5-amine (360 mg) and 1-hydroxybenzotriazol (253 mg). After cooling at 0° C., 1-ethyl-3-(3-dimethylaminopropyl)carbodiimide hydrochloride (897 mg) and N,N-diisopropylethylamine (0.815 mL) were added successively to the mixture and the mixture was stirred at 60° C. for 21 hours. To the mixture were added additional 1-ethyl-3-(3-dimethylaminopropyl)carbodiimide hydrochloride (449 mg) and N,N-diisopropylethylamine (0.815 mL), and the reaction mixture was stirred at 60° C. for 5 hours. Further additional 1-ethyl-3-(3-dimethylaminopropyl)carbodiimide hydrochloride (299 mg), 1-hydroxybenzotriazol (211 mg) and N,N-diisopropylethylamine (0.272 mL) were added to the mixture, and the reaction mixture was stirred at 60° C. for 1 hour. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-40/60) to give the title compound (202 mg) as a pale brown solid.

LC-MS: 474.2 (MH⁺).

### (ii) Production of tert-butyl[(1S)-2-({(1S)-1-cyclohexyl-2-[(3S,7R,8aR)-7-ethoxy-3-[(4-phenyl-1,2,3-thiadiazol-5-yl)carbamoyl]hexahydropyrrolo[1,2-a]pyrazin-2(1H)-yl]-2-oxoethyl}amino)-1-methyl-2-oxoethyl]methylcarbamate

A mixture of tert-butyl(3S,7R,8aR)-7-ethoxy-3-[(4-phenyl-1,2,3-thiadiazol-5-yl)carbamoyl]hexahydropyrrolo[1,2-a]pyrazine-2(1H)-carboxylate (202 mg) and 4M hydrogen chloride-ethyl acetate solution (3.0 mL) in methanol (2.0 mL) was stirred at room temperature for 2 hours. Additional 4M hydrogen chloride-ethyl acetate solution (1.0 mL) was added twice to the mixture and the reaction mixture was stirred at room temperature for additional 3 hours. The mixture was concentrated in vacuo. The residue was dissolved in N,N-dimethylformamide (3.0 mL), and to the solution were added (2S)-[(tert-butoxycarbonyl)amino](cyclohexyl)ethanoic acid (137 mg) and 1-hydroxybenzotriazol (69 mg). After cooling at 0° C., 1-ethyl-3-(3-dimethylaminopropyl)carbodiimide hydrochloride (164 mg) and N,N-diisopropylethylamine (0.335 mL) were added successively to the mixture, and the reaction mixture was stirred at room temperature for 18 hours. The mixture was diluted with water and extracted twice with ethyl acetate. The combined extracts were washed with saturated sodium hydrogen carbonate solution and brine, dried over anhydrous magnesium sulfate and concentrated in vacuo. The residue was purified by silica gel chromatography (eluent; ethyl acetate/hexane=10/90-50/50) to give tert-butyl{(1S)-1-cyclohexyl-2-[(3S,7R,8aR)-7-ethoxy-3-[(4-phenyl-1,2,3-thiadiazol-5-yl)carbamoyl]hexahydropyrrolo[1,2-a]pyrazin-2(1H)-yl]-2-oxoethyl}carbamate (247 mg) as a pale yellow solid. A mixture of the obtained pale yellow solid (247 mg) and 4M hydrogen chloride-ethyl acetate solution (4.0 mL) in methanol (2.0 mL) was stirred at room temperature for 1 hour. The mixture was concentrated in vacuo, and the residue was dissolved in N,N-dimethylformamide (4.0 mL). To the solution were added N-(tert-butoxycarbonyl)-N-methyl-L-alanine (90 mg) and 1-hydroxybenzotriazol (65 mg). After cooling at 0° C., 1-ethyl-3-(3-dimethylaminopropyl)carbodiimide hydrochloride (155 mg) and N,N-diisopropylethylamine (0.316 mL) were added successively to the mixture, and the reaction mixture was stirred at room temperature for 17 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=40/60-100/0) to give the title compound (228 mg) as a pale yellow solid.

LC-MS: 698.4 (MH⁺).

### (iii) Production of (3S,7R,8aR)-2-{(2S)-2-cyclohexyl-2-[(N-methyl-L-alanyl)amino]acetyl}-7-ethoxy-N-(4-phenyl-1,2,3-thiadiazol-5-yl)octahydropyrrolo[1,2-a]pyrazine-3-carboxamide

A solution of tert-butyl[(1S)-2-({(1S)-1-cyclohexyl-2-[(3S,7R,8aR)-7-ethoxy-3-[(4-phenyl-1,2,3-thiadiazol-5-yl)carbamoyl]hexahydropyrrolo[1,2-a]pyrazin-2(1H)-yl]-2-oxoethyl}amino)-1-methyl-2-oxoethyl]methylcarbamate (228 mg) in 4M hydrogen chloride-ethyl acetate solution (5.0 mL) was stirred at room temperature for 30 minutes. The mixture was concentrated in vacuo, and the residue treated with saturated sodium hydrogen carbonate solution and extracted three times with a mixed solvent of ethyl acetate-tetrahydrofuran (1:1). The combined extracts were washed with brine, dried over anhydrous magnesium sulfate and concentrated in vacuo. The residue was purified by silica gel chromatography (eluent; methanol/ethyl acetate=5/95-30/70) and recrystallized from ethyl acetate-hexane to give the title compound (154 mg) as a pale yellow solid.

LC-MS: 598.3 (MH⁺).

¹H NMR (300 MHz, DMSO-d₆): δ 0.71-1.35 (12H, m), 1.37-1.89 (7H, m), 1.94-2.25 (3H, m), 2.25-2.46 (3H, m), 2.68-3.07 (2H, m), 3.14-3.42 (4H, m), 3.49-4.18 (3H, m), 4.32-5.33 (2H, m), 6.96-7.61 (3H, m), 7.69-8.76 (4H, m).

### Example 108

### Production of (3S,7R,8aR)-2-{(2S)-2-cyclohexyl-2-[(N-methyl-L-alanyl)amino]acetyl}-7-ethoxy-N-(2,2,2-trifluoro-1-phenylethyl)octahydropyrrolo[1,2-a]pyrazine-3-carboxamide

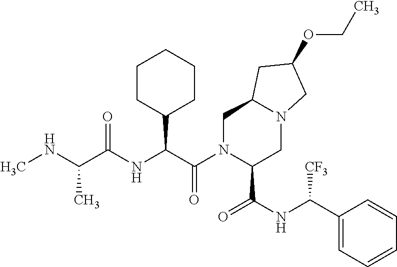

### (i) Production of tert-butyl{(1S)-1-cyclohexyl-2-[(3S,7R,8aR)-7-ethoxy-3-[(2,2,2-trifluoro-1-phenylethyl)carbamoyl]hexahydropyrrolo[1,2-a]pyrazin-2(1H)-yl]-2-oxoethyl}carbamate

A mixture of 2-tert-butyl 3-methyl(3S,7R,8aR)-7-ethoxyhexahydropyrrolo[1,2-a]pyrazine-2,3(1H)-dicarboxylate (500 mg) and lithium hydroxide monohydrate (102 mg) in tetrahydrofuran (10 mL)/water (2.0 mL) was stirred at 50° C. for 5 hours. The mixture was neutralized with 1N hydrochloric acid, concentrated in vacuo and the residue was subjected to azeotropic distillation with toluene. The residue was dissolved in N,N-dimethylformamide (3.0 mL), and to the solution were added 2,2,2-trifluoro-1-phenylethanamine (233 mg) and 1-hydroxybenzotriazol (165 mg). After cooling at 0° C., 1-ethyl-3-(3-dimethylaminopropyl)carbodiimide hydrochloride (391 mg) and N,N-diisopropylethylamine (0.622 mL) were added successively to the mixture, and the reaction mixture was stirred at room temperature for 1 day. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-50/50) to give tert-butyl(3S,7R,8aR)-7-ethoxy-3-[(2,2,2-trifluoro-1-phenylethyl)carbamoyl]hexahydropyrrolo[1,2-a]pyrazine-2(1H)-carboxylate (450 mg) as a white solid. A solution of the obtained white solid (450 mg) in 4M hydrogen chloride-cyclopentyl methyl ether solution (4.0 mL) was stirred at room temperature for 2 hours. The mixture was concentrated in vacuo. The residue was dissolved in N,N-dimethylformamide (7.0 mL), and to the solution were added (2S)-[(tert-butoxycarbonyl)amino](cyclohexyl)ethanoic acid (270 mg) and 1-hydroxybenzotriazol (155 mg). After cooling at 0° C., 1-ethyl-3-(3-dimethylaminopropyl)carbodiimide hydrochloride (366 mg) and N,N-diisopropylethylamine (0.748 mL) were added successively to the solution, and the reaction mixture was stirred at room temperature for 18 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-70/30) to give the title compound (437 mg) as pale yellow oil.

LC-MS: 611.4 (MH⁺).

### (ii) Production of tert-butyl[(1S)-2-({(1S)-1-cyclohexyl-2-[(3S,7R,8aR)-7-ethoxy-3-[(2,2,2-trifluoro-1-phenylethyl)carbamoyl]hexahydropyrrolo[1,2-a]pyrazin-2(1H)-yl]-2-oxoethyl}amino)-1-methyl-2-oxoethyl]methylcarbamate

A solution of tert-butyl{(1S)-1-cyclohexyl-2-[(3S,7R,8aR)-7-ethoxy-3-[(2,2,2-trifluoro-1-phenylethyl)carbamoyl]hexahydropyrrolo[1,2-a]pyrazin-2(1H)-yl]-2-oxoethyl}carbamate (435 mg) in 4M hydrogen chloride-cyclopentyl methyl ether solution (4.0 mL) was stirred at room temperature for 3 hours. The mixture was concentrated in vacuo. The residue was dissolved in N,N-dimethylformamide (6.0 mL), and to the solution were added N-(tert-butoxycarbonyl)-N-methyl-L-alanine (159 mg) and 1-hydroxybenzotriazol (115 mg). After cooling at 0° C., 1-ethyl-3-(3-dimethylaminopropyl)carbodiimide hydrochloride (273 mg) and N,N-diisopropylethylamine (0.558 mL) were added successively to the mixture, and the reaction mixture was stirred at room temperature for 18 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=40/60-100/0) to give the title compound (417 mg) as pale yellow oil.

LC-MS: 696.4 (MH⁺).

### (iii) Production of (3S,7R,8aR)-2-{(2S)-2-cyclohexyl-2-[(N-methyl-L-alanyl)amino]acetyl}-7-ethoxy-N-(2,2,2-trifluoro-1-phenylethyl)octahydropyrrolo[1,2-a]pyrazine-3-carboxamide

A solution of tert-butyl[(1S)-2-({(1S)-1-cyclohexyl-2-[(3S,7R,8aR)-7-ethoxy-3-[(2,2,2-trifluoro-1-phenylethyl)carbamoyl]hexahydropyrrolo[1,2-a]pyrazin-2(1H)-yl]-2-oxoethyl}amino)-1-methyl-2-oxoethyl]methylcarbamate (409 mg) in 4M hydrogen chloride-ethyl acetate solution (5.0 mL) was stirred at room temperature for 1.5 hours. The mixture was concentrated in vacuo, and the residue was treated with saturated sodium hydrogen carbonate solution and extracted twice with ethyl acetate. The combined extracts were washed with brine, dried over anhydrous magnesium sulfate and concentrated in vacuo. The residue was purified by silica gel chromatography (eluent; methanol/ethyl acetate=5/95-30/70) and recrystallized from ethyl acetate-hexane to give the title compound (255 mg) as a white solid.

LC-MS: 596.4 (MH⁺).

¹H NMR (300 MHz, DMSO-d₆): δ 0.75-1.19 (11H, m), 1.22-1.40 (1H, m), 1.42-1.78 (6H, m), 1.79-1.97 (1H, m), 2.04-2.31 (6H, m), 2.80-3.03 (2H, m), 3.22-3.55 (5H, m), 3.84-4.04 (1H, m), 4.06-4.44 (1H, m), 4.50-4.76 (1H, m), 4.85-5.16 (1H, m), 5.64-5.88 (1H, m), 7.35-7.49 (3H, m), 7.51-7.62 (2H, m), 7.91 (1H, d, J=8.7 Hz), 9.03-9.46 (1H, m).

### Example 109

### Production of N-{(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}-N-2-methyl-L-alaninamide dihydrochloride

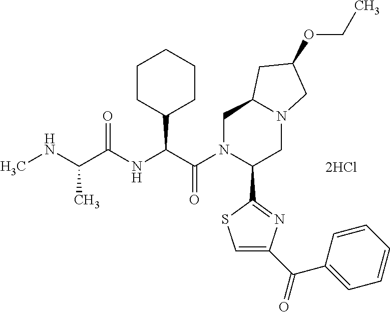

### (i) Production of tert-butyl(3S,7R,8aR)-3-carbamoyl-7-ethoxyhexahydropyrrolo[1,2-a]pyrazine-2(1H)-carboxylate

A mixture of 2-tert-butyl 3-methyl(3S,7R,8aR)-7-ethoxyhexahydropyrrolo[1,2-a]pyrazine-2,3(1H)-dicarboxylate (8.21 g) and lithium hydroxide monohydrate (2.10 g) in tetrahydrofuran (200 mL)/water (40 mL) was stirred at 50° C. for 4.5 hours. The mixture was neutralized with 2M hydrochloric acid, and concentrated in vacuo. The residue was dissolved in N,N-dimethylformamide (80 mL), and to the solution was added 1H-benzotriazol-1-01 ammoniate (5.71 g). After cooling at 0° C., 1-ethyl-3-(3-dimethylaminopropyl)carbodiimide (7.76 g) was added to the mixture, and the reaction mixture was stirred at room temperature for 63.5 hours. The mixture was diluted with water and extracted three times with ethyl acetate. The combined extracts were washed with brine, dried over anhydrous magnesium sulfate and concentrated in vacuo. The residue was purified by silica gel chromatography (eluent; ethyl acetate/hexane=60/40-100/0) to give the title compound (5.87 g) as pale brown oil.

LC-MS: 314.2 (MH⁺).

### (ii) Production of tert-butyl(3S,7R,8aR)-7-ethoxy-3-[4-(ethoxycarbonyl)-1,3-thiazol-2-yl]hexahydropyrrolo[1,2-a]pyrazine-2(1H)-carboxylate

A mixture of tert-butyl(3S,7R,8aR)-3-carbamoyl-7-ethoxyhexahydropyrrolo[1,2-a]pyrazine-2(1H)-carboxylate (5.87 g) and 2,4-bis(4-methoxyphenyl)-1,3,2,4-dithiadiphosphetane 2,4-disulfide (8.33 g) in 1,2-dimethoxyethane (60 mL) was stirred at 60° C. for 17 hours. The mixture was diluted with saturated sodium hydrogen carbonate solution and extracted twice with ethyl acetate. The combined extracts were washed with brine, dried over anhydrous magnesium sulfate and concentrated in vacuo. The residue was purified by basic silica gel chromatography (eluent; ethyl acetate/hexane=10/90-70/30) to give tert-butyl(3S,7R,8aR)-3-carbamothioyl-7-ethoxyhexahydropyrrolo[1,2-a]pyrazine-2(1H)-carboxylate (3.42 g) as a pale yellow solid. A mixture of the obtained pale yellow solid (2.20 g), ethyl 3-bromo-2-oxopropanoate (2.5 mL) and potassium bicarbonate (5.35 g) in 1,2-dimethoxyethane (22 mL) was stirred at room temperature for 1.5 hours. To the mixture were added 2,2,2-trifluoroacetic anhydride (3.7 mL) and 2,4,6-trimethylpyridine (7.1 mL) at 0° C., and the reaction mixture was stirred at room temperature for 18 hours. Additional 2,2,2-trifluoroacetic anhydride (1.9 mL) and 2,4,6-trimethylpyridine (3.6 mL) were added to the mixture at 0° C. and the reaction mixture was stirred at room temperature for 1 hour, and at 40-60° C. for 4 hours. Additional 2,2,2-trifluoroacetic anhydride (1.9 mL) and 2,4,6-trimethylpyridine (3.6 mL) were added to the mixture at 0° C. and the mixture was stirred at room temperature for 64 hours. Further additional 2,2,2-trifluoroacetic anhydride (1.9 mL) and 2,4,6-trimethylpyridine (1.8 mL) were added to the mixture at 0° C. and the mixture was stirred at room temperature for 1.5 hours. The reaction mixture was diluted with water and extracted four times with ethyl acetate. The combined extracts were washed with brine, dried over anhydrous magnesium sulfate and concentrated in vacuo. The residue was purified by silica gel chromatography (eluent; ethyl acetate/hexane=10/90-30/70) and basic silica gel chromatography (eluent; ethyl acetate/hexane=5/95-30/70) to give the title compound (2.39 g) as pale yellow oil.

LC-MS: 426.2 (MH⁺).

### (iii) Production of tert-butyl(3S,7R,8aR)-7-ethoxy-3-{4-[methoxy(methyl)carbamoyl]-1,3-thiazol-2-yl}hexahydropyrrolo[1,2-a]pyrazine-2(1H)-carboxylate

A mixture of tert-butyl(3S,7R,8aR)-7-ethoxy-3-[4-(ethoxycarbonyl)-1,3-thiazol-2-yl]hexahydropyrrolo[1,2-a]pyrazine-2(1H)-carboxylate (2.39 g) and lithium hydroxide monohydrate (471 mg) in tetrahydrofuran (24 mL)/water (4.8 mL) was stirred at 50° C. for 1 hour. After cooling to 0° C., the mixture was neutralized with 2N hydrochloric acid, and concentrated in vacuo. The residue was dissolved in N,N-dimethylformamide (24 mL), and to the solution were added N-methoxymethanamine (1.62 g), 1-hydroxybenzotriazol (1.33 g). After cooling at 0° C., N,N-diisopropylethylamine (1.96 mL) and 1-ethyl-3-(3-dimethylaminopropyl)carbodiimide (1.53 g) were added successively to the mixture, and the reaction mixture was stirred at room temperature for 17.5 hours. The mixture was diluted with water and extracted three times with ethyl acetate. The combined extracts were washed with brine, dried over anhydrous magnesium sulfate and concentrated in vacuo. The residue was purified by silica gel chromatography (eluent; ethyl acetate/hexane=40/60-100/0) and basic silica gel chromatography (eluent; ethyl acetate/hexane=5/95-50/50) to give the title compound (2.28 g) as colorless oil.

LC-MS: 441.2

### (iv) Production of tert-butyl(3S,7R,8aR)-7-ethoxy-3-[4-(phenylcarbonyl)-1,3-thiazol-2-yl]hexahydropyrrolo[1,2-a]pyrazine-2(1H)-carboxylate

To a solution of tert-butyl(3S,7R,8aR)-7-ethoxy-3-[(4-[methoxy(methyl)carbamoyl]-1,3-thiazol-2-yl]hexahydropyrrolo[1,2-a]pyrazine-2(1H)-carboxylate (124 mg) in tetrahydrofuran (2.0 mL) was added 1N phenylmagnesium bromide tetrahydrofuran solution (0.338 mL) at 0° C., and the reaction mixture was stirred at room temperature for 17.5 hours. The mixture was diluted with saturated ammonium chloride solution and extracted twice with, ethyl acetate. The combined extracts were washed with brine, dried over anhydrous magnesium sulfate and concentrated in vacuo. The residue was purified by basic silica gel chromatography (eluent; ethyl acetate/hexane=5/95-30/70) and silica gel chromatography (eluent; ethyl acetate/hexane=10/90-30/70) to give the title to compound (88 mg) as pale yellow oil.

LC-MS: 458.2 (MH⁺).

### (v) Production of tert-butyl{(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}carbamate

A mixture of tert-butyl(3S,7R,8aR)-7-ethoxy-3-[4-(phenylcarbonyl)-1,3-thiazol-2-yl]hexahydropyrrolo[1,2-a]pyrazine-2(1H)-carboxylate (85 mg) and 4M hydrogen chloride-cyclopentyl methyl ether solution (3.0 mL) in ethyl acetate (1.0 mL) was stirred at room temperature for 1.5 hours. The mixture was concentrated in vacuo. The residue was dissolved in N,N-dimethylformamide (3.0 mL), and to the solution was added (2S)-[(tert-butoxycarbonyl)amino](cyclohexyl)ethanoic acid (57 mg). After cooling at 0° C., O-(7-azabenzotriazol-1-yl)-N,N,N′,N′-tetramethyluronium hexafluorophosphate (106 mg) and N,N-diisopropylethylamine (0.097 mL) were added successively to the mixture, and the reaction mixture was stirred at room temperature for 14.5 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=5/95-30/70) to give the title compound (93 mg) as pale yellow oil.

LC-MS: 597.3 (MH⁺).

### (vi) Production of tert-butyl[(1S)-2-({(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}amino)-1-methyl-2-oxoethyl]methylcarbamate

A mixture of tert-butyl{(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}carbamate (90 mg) and 4M hydrogen chloride-cyclopentyl methyl ether solution (3.0 mL) in ethyl acetate (1.0 mL)/methanol (0.50 mL) was stirred at room temperature for 2.5 hours. The mixture was concentrated in vacuo. The residue was dissolved in N,N-dimethylformamide (3.0 mL), and to the solution was added N-(tert-butoxycarbonyl)-N-methyl-L-alanine (37 mg). After cooling at 0° C., O-(7-azabenzotriazol-1-yl)-N,N,N′,N′-tetramethyluronium hexafluorophosphate (86 mg) and N,N-diisopropylethylamine (0.079 mL) were added successively to the mixture, and the reaction mixture was stirred at room temperature for 64 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=20/80-100/0) to give the title compound (57 mg) as a colorless oil.

LC-MS: 682.3 (MH⁺).

### (vii) Production of N-{(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}-N-2-methyl-L-alaninamide dihydrochloride

A mixture of tert-butyl[(1S)-2-({(1S)-1-cyclohexyl-2-[(3S,7R,8aR)-7-ethoxy-3-[4-(phenylcarbonyl)-1,3-thiazol-2-yl]hexahydropyrrolo[1,2-a]pyrazin-2(1H)-yl]-2-oxoethyl}amino)-1-methyl-2-oxoethyl]methylcarbamate (55 mg) and 4M hydrogen chloride-cyclopentyl methyl ether solution (4.0 mL) in ethyl acetate (1.5 mL) was stirred at room temperature for 1 hour. The mixture was concentrated in vacuo and the residue was precipitated from tetrahydrofuran-hexane to give the title compound (29 mg) as a pale brown solid.

LC-MS: 582.3 (MH⁺).

¹H NMR (300 MHz, DMSO-d₆): [symbol] 0.83-1.80 (22H, m), 1.85-2.14 (1H, m), 2.18-2.42 (3H, m), 2.59-2.71 (3H, m), 3.66-4.28 (4H, m), 4.43-4.98 (2H, m), 7.42-7.62 (3H, m), 7.64-7.77 (1H, m), 7.99-8.25 (2H, m), 8.47-9.44 (4H, m).

### Example 110

### Production of N-{(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

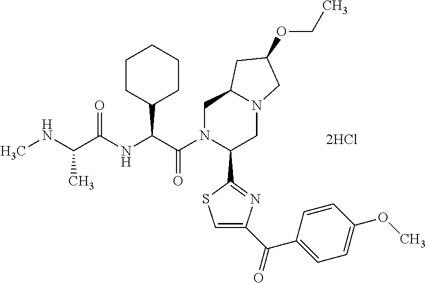

### (i) Production of tert-butyl(3S,7R,8aR)-7-ethoxy-3-{4-[(4-methoxyphenyl)carbonyl]-1,3-thiazol-2-yl}hexahydropyrrolo[1,2-a]pyrazine-2(1H)-carboxylate

To a solution of tert-butyl(3S,7R,8aR)-7-ethoxy-3-{4-[methoxy(methyl)carbamoyl]-1,3-thiazol-2-yl}hexahydropyrrolo[1,2-a]pyrazine-2(1H)-carboxylate (250 mg) in tetrahydrofuran (5.0 mL) was added 0.5M 4-methoxyphenylmagnesium bromide tetrahydrofuran solution (1.7 mL) at 0° C., and the mixture was stirred at 0° C. for 2 hours. Additional 0.5M 4-methoxyphenylmagnesium bromide tetrahydrofuran solution (2.5 mL) was added to the mixture at 0° C., and the reaction mixture was stirred at 0° C. for 30 minutes. Further additional 0.5M 4-methoxyphenylmagnesium bromide tetrahydrofuran solution (1.7 mL) was added to the mixture at 0° C., and the reaction mixture was stirred at 0° C. for 30 minutes. The reaction mixture was diluted with saturated ammonium chloride solution and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-50/50) to give the title compound (265 mg) as a colorless solid.

LC-MS: 488.2 (MH⁺).

### (ii) Production of tert-butyl{(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl}carbamate

A mixture of tert-butyl(3S,7R,8aR)-7-ethoxy-3-{4-[(4-methoxyphenyl)carbonyl]-1,3-thiazol-2-yl}hexahydropyrrolo[1,2-a]pyrazine-2(1H)-carboxylate (265 mg) and 4M hydrogen chloride-cyclopentyl methyl ether solution (5.0 mL) in ethyl acetate (1.0 mL)/methanol (3.0 mL) was stirred at room temperature for 1.5 hours. The mixture was concentrated in vacuo. The residue was dissolved in N,N-dimethylformamide (5.0 mL), and to the solution were added (2S)-[(tert-butoxycarbonyl)amino](cyclohexyl)ethanoic acid (168 mg) and N,N-diisopropylethylamine (0.284 mL). After cooling at 0° C., O-(7-azabenzotriazol-1-yl)-N,N,N′,N′-tetramethyluronium hexafluorophosphate (310 mg) was added to the mixture, and the reaction mixture was stirred at room temperature for 15 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-50/50) to give the title compound (207 mg) as a colorless solid.

LC-MS: 627.3 (MH⁺).

### (iii) Production of tert-butyl[(1S)-2-({(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl}amino)-1-methyl-2-oxoethyl]methylcarbamate

A mixture of tert-butyl{(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl}carbamate (207 mg) and 4M hydrogen chloride-cyclopentyl methyl ether solution (2.0 mL) in ethyl acetate (0.50 mL)/methanol (0.50 mL) was stirred at room temperature for 6 hours. The mixture was concentrated in vacuo. The residue was dissolved in N,N-dimethylformamide (4.0 mL), and to the solution were added N-(tert-butoxycarbonyl)-N-methyl-L-alanine (81 mg) and N,N-diisopropylethylamine (0.172 mL). After cooling at 0° C., O-(7-azabenzotriazol-1-yl)-N,N,N′,N′-tetramethyluronium hexafluorophosphate (188 mg) was added to the mixture, and the reaction mixture was stirred at room temperature for 16.5 hours. The mixture was diluted with water and extracted twice with ethyl acetate. The extracts were washed with brine, dried over anhydrous magnesium sulfate and concentrated in vacuo. The residue was purified by silica gel chromatography (eluent; ethyl acetate/hexane=20/80-100/0) to give the title compound (250 mg) as pale yellow oil.

LC-MS: 712.4 (MH⁺).

### (iv) Production of N-{(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

A mixture of tert-butyl[(1S)-2-({(1S)-1-cyclohexyl-2-[(3S,7R,8aR)-7-ethoxy-3-{4-[(4-methoxyphenyl)carbonyl]-1,3-thiazol-2-yl}hexahydropyrrolo[1,2-a]pyrazin-2(1H)-yl]-2-oxoethyl)amino)-1-methyl-2-oxoethyl]methylcarbamate (235 mg) and 4M hydrogen chloride-cyclopentyl methyl ether solution (2.0 mL) in ethyl acetate (1.0 mL)/methanol (1.0 mL) was stirred at room temperature for 2.5 hours. The mixture was concentrated in vacuo, and the residue was treated with saturated sodium hydrogen carbonate solution and extracted twice with a mixed solvent of ethyl acetate-tetrahydrofuran (1:1). The combined extracts were washed with brine, dried over anhydrous magnesium sulfate and concentrated in vacuo. The residue was purified by silica gel chromatography (eluent; methanol/ethyl acetate=2/98-30/70). The purified fractions were concentrated in vacuo and the residue was dissolved in methanol (1.0 mL). The solution was treated with 4M hydrogen chloride-cyclopentyl methyl ether solution (1.0 mL) and concentrated in vacuo. The residue was precipitated from methanol-diethyl ether to give the title compound (114 mg) as a pale brown solid.

LC-MS: 612.3 (MH⁺).

¹H NMR (300 MHz, DMSO-d₆): [symbol] 0.65-1.78 (19H, m), 1.92-2.14 (1H, m), 2.19-2.42 (2H, m), 2.43-2.56 (3H, m), 3.26-3.61 (3H, m), 3.63-3.82 (1H, m), 3.82-3.93 (4H, m), 3.95-4.27 (2H, m), 4.44-5.03 (2H, m), 5.55-5.96 (1H, m), 6.81-7.24 (2H, m), 7.96-8.35 (2H, m), 8.47-8.63 (1H, m), 8.64-9.40 (3H, m), 12.35 (1H, br. s.).

### Example 111

### Production of N-{(1S)-1-cyclohexyl-2-[(3S,7R,8aR)-7-ethoxy-3-{4-[(3-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

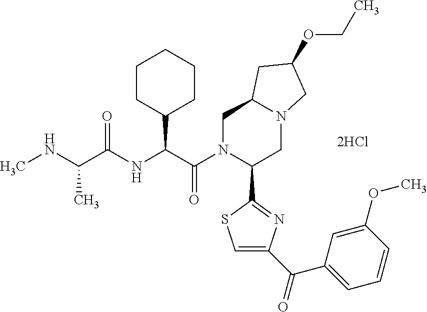

### (i) Production of tert-butyl(3S,7R,8aR)-7-ethoxy-3-{4-[(3-methoxyphenyl)carbonyl]-1,3-thiazol-2-yl}hexahydropyrrolo[1,2-a]pyrazine-2(1H)-carboxylate

To a solution of tert-butyl(3S,7R,8aR)-7-ethoxy-3-{(4-[methoxy(methyl)carbamoyl]-1,3-thiazol-2-yl}hexahydropyrrolo[1,2-a]pyrazine-2(1H)-carboxylate (250 mg) in tetrahydrofuran (5.0 mL) was added 1M 3-methoxyphenylmagnesium bromide tetrahydrofuran solution (0.851 mL) at 0° C., and the reaction mixture was stirred at 0° C. for 2 hours. Additional 1N 3-methoxyphenylmagnesium bromide tetrahydrofuran solution (0.851 mL) was added to the mixture at 0° C., and the mixture was stirred at 0° C. for 30 minutes. The reaction mixture was diluted with saturated ammonium chloride solution and extracted with ethyl acetate. The organic extract was washed with brine, dried over anhydrous magnesium sulfate and concentrated in vacuo. The residue was purified by silica gel chromatography (eluent; ethyl acetate/hexane=10/90-50/50) to give the title compound (244 mg) as a pale yellow solid.

LC-MS: 488.2 (MH⁺).

### (ii) Production of tert-butyl{(1S)-1-cyclohexyl-2-[(3S,7R,8aR)-7-ethoxy-3-[(4-[(3-methoxyphenyl)carbonyl]-1,3-thiazol-2-yl]hexahydropyrrolo[1,2-a]pyrazin-2(1H)-yl]-2-oxoethyl}carbamate

A mixture of tert-butyl(3S,7R,8aR)-7-ethoxy-3-{4-[(3-methoxyphenyl)carbonyl]-1,3-thiazol-2-yl}hexahydropyrrolo[1,2-a]pyrazine-2(1H)-carboxylate (244 mg) and 4M hydrogen chloride-cyclopentyl methyl ether solution (5.0 mL) in ethyl acetate (1.0 mL)/methanol (3.0 mL) was stirred at room temperature for 1.5 hours. The reaction mixture was concentrated in vacuo, and the residue was dissolved in N,N-dimethylformamide (5.0 mL). To the solution were added (2S)-[(tert-butoxycarbonyl)amino](cyclohexyl)ethanoic acid (155 mg) m and N,N-diisopropylethylamine (0.097 mL). After cooling at 0° C., O-(7-azabenzotriazol-1-yl)-N,N,N′,N′-tetramethyluronium hexafluorophosphate (285 mg) was added to the mixture, and the reaction mixture was stirred at room temperature for 16 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-50/50) to give the title compound (206 mg) as a pale yellow solid.

LC-MS: 627.3 (MH⁺).

### (iii) Production of tert-butyl[(1S)-2-({(1S)-1-cyclohexyl-2-[(3S,7R,8aR)-7-ethoxy-3-[(4-[(3-methoxyphenyl)carbonyl]-1,3-thiazol-2-yl]hexahydropyrrolo[1,2-a]pyrazin-2(1H)-yl]-2-oxoethyl}amino)-1-methyl-2-oxoethyl]methylcarbamate

A mixture of tert-butyl{(1S)-1-cyclohexyl-2-[(3S,7R,8aR)-7-ethoxy-3-{4-[(3-methoxyphenyl)carbonyl]-1,3-thiazol-2-yl}hexahydropyrrolo[1,2-a]pyrazin-2(1H)-yl]-2-oxoethyl}carbamate (206 mg) and 4M hydrogen chloride-cyclopentyl methyl ether solution (2.0 mL) in ethyl acetate (0.50 mL)/methanol (0.50 mL) was stirred at room temperature for 6 hours. The mixture was concentrated in vacuo, and the residue was dissolved in N,N-dimethylformamide (4.0 mL). To the solution were added N-(tert-butoxycarbonyl)-N-methyl-L-alanine (80 mg) and N,N-diisopropylethylamine (0.172 mL). After cooling at 0° C., O-(7-azabenzotriazol-1-yl)-N,N,N′,N′-tetramethyluronium hexafluorophosphate (188 mg) was added to the mixture, and the reaction mixture was stirred at room temperature for 17 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=20/80-100/0) to give the title compound (270 mg) as pale yellow oil.

LC-MS: 712.4 (MH⁺).

### (iv) Production of N-{(1S)-1-cyclohexyl-2-[(3S,7R,8aR)-7-ethoxy-3-{4-[(3-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

A mixture of tert-butyl[(1S)-2-([(1S)-1-cyclohexyl-2-[(3S,7R,8aR)-7-ethoxy-3-{4-[(3-methoxyphenyl)carbonyl]-1,3-thiazol-2-yl]hexahydropyrrolo[1,2-a]pyrazin-2(1H)-yl]-2-oxoethyl}amino)-1-methyl-2-oxoethyl]methylcarbamate (234 mg) and 4M hydrogen chloride-cyclopentyl methyl ether solution (2.0 mL) in ethyl acetate (1.0 mL)/methanol (1.0 mL) was stirred at room temperature for 2 hours. The mixture was concentrated in vacuo, and the residue was treated with saturated sodium hydrogen carbonate solution and extracted twice with a mixed solvent of ethyl acetate-tetrahydrofuran (1:1). The combined extracts were washed with brine, dried over anhydrous magnesium sulfate and concentrated in vacuo. The residue was purified by silica gel chromatography (eluent; methanol/ethyl acetate=2/98-30/70). The purified fractions were concentrated in vacuo and the residue was dissolved in methanol (1.0 mL). 4M Hydrogen chloride-cyclopentyl methyl ether solution (1.0 mL) was added to the solution, and the mixture was concentrated in vacuo. The residue was precipitated from methanol-diethyl ether to give the title compound (103 mg) as a pale yellow solid.

LC-MS: 612.3 (MH⁺).

¹H NMR (300 MHz, DMSO-d₆): δ 0.71-1.79 (19H, m), 1.92-2.11 (1H, m), 2.19-2.41 (2H, m), 2.41-2.57 (2H, m), 3.19-3.61 (3H, m), 3.66-4.28 (8H, m), 4.45-5.01 (2H, m), 5.64-5.90 (1H, m), 7.11-7.35 (1H, m), 7.47 (1H, t, J=7.9 Hz), 7.53-7.85 (2H, m), 8.51-9.04 (3H, m), 9.10-9.62 (1H, m), 12.38 (1H, br. s.).

### Example 112

### Production of N-{(1S)-1-cyclohexyl-2-[(3S,7R,8aR)-7-ethoxy-3-{4-[(2-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

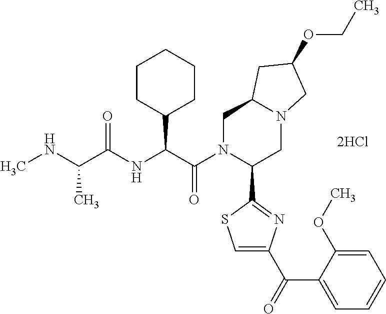

### (i) Production of tert-butyl(3S,7R,8aR)-7-ethoxy-3-{4-[(2-methoxyphenyl)carbonyl]-1,3-thiazol-2-yl}hexahydropyrrolo[1,2-a]pyrazine-2(1H)-carboxylate

To a solution of tert-butyl(3S,7R,8aR)-7-ethoxy-3-{4-[methoxy(methyl)carbamoyl]-1,3-thiazol-2-yl}hexahydropyrrolo[1,2-a]pyrazine-2(1H)-carboxylate (250 mg) in tetrahydrofuran (5.0 mL) was added 1M 2-methoxyphenylmagnesium bromide tetrahydrofuran solution (0.851 mL) at 0° C., and the reaction mixture was stirred at 0° C. for 2 hours. Additional 1M 2-methoxyphenylmagnesium bromide tetrahydrofuran solution (0.851 mL) was added to the mixture at 0° C., and the reaction mixture was stirred at 0° C. for 30 minutes. Further additional 1M 2-methoxyphenylmagnesium bromide tetrahydrofuran solution (0.851 mL) was added to the mixture at 0° C., and the reaction mixture was stirred at room temperature for 1 day. The mixture was diluted with saturated ammonium chloride solution and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-50/50) to give the title compound (224 mg) as a pale yellow solid.

LC-MS: 488.2 (MH⁺).

### (ii) Production of tert-butyl[(1S)-2-({(1S)-1-cyclohexyl-2-[(3S,7R,8aR)-7-ethoxy-3-{4-[(2-methoxyphenyl)carbonyl]-1,3-thiazol-2-yl}hexahydropyrrolo[1,2-a]pyrazin-2(1H)-yl]-2-oxoethyl}amino)-1-methyl-2-oxoethyl]methylcarbamate

A mixture of tert-butyl(3S,7R,8aR)-7-ethoxy-3-{4-[(2-methoxyphenyl)carbonyl]-1,3-thiazol-2-yl}hexahydropyrrolo[1,2-a]pyrazine-2(1H)-carboxylate (224 mg) and 4M hydrogen chloride-cyclopentyl methyl ether solution (4.0 mL) in ethyl acetate (1.0 mL)/methanol (1.0 mL) was stirred at room temperature for 1.5 hours. The mixture was concentrated in vacuo. The residue was dissolved in N,N-dimethylformamide (5.0 mL), and to the solution were added (2S)-[(tert-butoxycarbonyl)amino](cyclohexyl)ethanoic acid (142 mg) and N,N-diisopropylethylamine (0.24 mL). After cooling at 0° C., O-(7-azabenzotriazol-1-yl)-N,N,N′,N′-tetramethyluronium hexafluorophosphate (262 mg) was added to the mixture, and the reaction mixture was stirred at room temperature for 17 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-40/60) to give tert-butyl{(1S)-1-cyclohexyl-2-[(3S,7R,8aR)-7-ethoxy-3-{4-[(2-methoxyphenyl)carbonyl]-1,3-thiazol-2-yl}hexahydropyrrolo[1,2-a]pyrazin-2(1H)-yl]-2-oxoethyl}carbamate (142 mg) as a pale yellow solid. A mixture of the obtained pale yellow solid (147 mg) and 4M hydrogen chloride-cyclopentyl methyl ether solution (2.0 mL) in ethyl acetate (0.50 mL)/methanol (0.50 mL) was stirred at room temperature for 3 hours. The mixture was concentrated in vacuo. The residue was dissolved in N,N-dimethylformamide (2.0 mL), and to the solution were added N-(tert-butoxycarbonyl)-N-methyl-L-alanine (57 mg) and N,N-diisopropylethylamine (0.123 mL). After cooling at 0° C., O-(7-azabenzotriazol-1-yl)-N,N,N′,N′-tetramethyluronium hexafluorophosphate (134 mg) was added to the mixture, and the reaction mixture was stirred at room temperature for 17 hours. The mixture was diluted with water and extracted twice with ethyl acetate. The combined extracts were washed with brine, dried over anhydrous magnesium sulfate and concentrated in vacuo. The residue was purified by silica gel chromatography (eluent; ethyl acetate/hexane=40/60-100/0) to give the title compound (225 mg) as brown oil.

LC-MS: 712.4 (MH⁺).

### (iii) Production of N-{(1S)-1-cyclohexyl-2-[(3S,7R,8aR)-7-ethoxy-3-{4-[(2-methoxyphenyl)carbonyl]-1,3-thiazol-2-yl}hexahydropyrrolo[1,2-a]pyrazin-2(1H)-yl]-2-oxoethyl}-N-2-methyl-L-alaninamide dihydrochloride

A mixture of tert-butyl[(1S)-2-({(1S)-1-cyclohexyl-2-[(3S,7R,8aR)-7-ethoxy-3-{4-[(2-methoxyphenyl)carbonyl]-1,3-thiazol-2-yl}hexahydropyrrolo[1,2-a]pyrazin-2(1H)-yl]-2-oxoethyl}amino)-1-methyl-2-oxoethyl]methylcarbamate (167 mg) and 4M hydrogen chloride-cyclopentyl methyl ether solution (2.0 mL) in ethyl acetate (0.50 mL)/methanol (0.50 mL) was stirred at room temperature for 3 hours. The mixture was concentrated in vacuo, and the residue was treated with saturated sodium hydrogen carbonate solution and extracted twice with a mixed solvent of ethyl acetate-tetrahydrofuran (1:1). The extracts were washed with brine, dried over anhydrous magnesium sulfate and concentrated in vacuo. The residue was purified by silica gel chromatography (eluent; methanol/ethyl acetate=2/98-30/70). The purified fractions were concentrated in vacuo and the residue was dissolved in 10% hydrogen chloride-methanol solution (1.0 mL). To the solution was added ethyl acetate and the mixture was stirred at 0° C. Diethyl ether was added dropwise to the solution, and the resulting precipitate was collected by filtration and dried under vacuum to give the title compound (43 mg) as a pale yellow solid.

LC-MS: 612.3 (MH⁺).

¹H NMR (300 MHz, DMSO-d₆): δ 0.79-1.81 (19H, m), 1.86-2.10 (1H, m), 2.20-2.49 (5H, m), 3.25-3.45 (2H, m), 3.59-3.78 (4H, m), 3.79-4.05 (3H, m), 4.07-4.28 (1H, m), 4.34-4.92 (2H, m), 5.46-5.90 (1H, m), 6.95-7.11 (I H, m), 7.11-7.26 (1H, m), 7.28-7.46 (1H, m), 7.47-7.63 (1H, m), 8.14-8.49 (1H, m), 8.52-9.11 (2H, m), 9.30 (1 H, br s), 12.29 (1H, br s).

### Example 113-Example 124

In the similar manner to that in Example 113, the compounds shown in Table 2 were synthesized.

### <LC-MS for Example 113-Example 118, Example 120-Example 124>

Apparatus: Waters MUX 4-ch LC/MS system
Column: CAPCELL PAK c18UG120 S-3 μm, 1.5×35 mm (Shisendo)
Solvent: phase A: 5 mM ammonium acetate water solution
phase B: 5 mM ammonium acetate acetonitrile solution
Gradient Cycle: 0 min. (phase A/phase B=100/0), 2.00 min. (phase A/phase B-0/100), 3.00 min. (phase A/phase B=0/100), 3.01 min. (phase A/phase B=100/0), 3.30 min. (phase A/phase B=100/0)
Injection volume: 2 μL
Flow rate: 0.5 mL/min

### Detection Wavelength: UV 220 nm

Ionization method: ESI
Measurement mode: Full Scan (positive and negative)
MS range: m/z=150-750

### <LC-MS for Example 119>

Apparatus: Waters ZMD system
Column: CAPCELL PAK c18UG120 S-3 μm, 1.5×35 mm (Shisendo)
Solvent: phase A: 0.05% TFA water solution
phase B: 0.05% TFA acetonitrile solution
Gradient Cycle: 0 min. (phase A/phase B=90/10), 2.00 min. (phase A/phase B=90/10), 2.75 min. (phase A/phase B=5/95), 3.45 min. (phase A/phase B=90/10)
Injection volume: 2 μL
Flow rate: 0.5 mL/min

### Detection Wavelength: UV 220 nm

Ionization method: ESI
Measurement mode: Full Scan (positive)
MS range: m/z=200-800

### <Preparative HPLC>

### Apparatus: GILSON High Throughput Purification System

Column: YMC combiprep pro C18 RS (20 mmI.D.×50 mm, 5 μm)
Solvent: phase A: 10 mM ammonium bicarbonate water solution phase B: acetonitrile
Gradient Cycle: 0 min. (phase A/phase B=95/5), 1.10 min. (phase A/phase B=95/5), 5.00 min. (phase A/phase B=0/100), 6.40 min. (phase A/phase B=0/100), 6.50 min. (phase A/phase B=95/5)
Flow rate: 20 mL/min

### Detection Wavelength: UV 220,254 nm

### Example 113

### Production of N-{(1S)-1-cyclohexyl-2-[(3S,8aR)-3-[4-(furan-3-ylcarbonyl)-1,3-thiazol-2-yl]hexahydropyrrolo[1,2-a]pyrazin-2(1H)-yl]-2-oxoethyl}-N-2-methyl-L-alaninamide

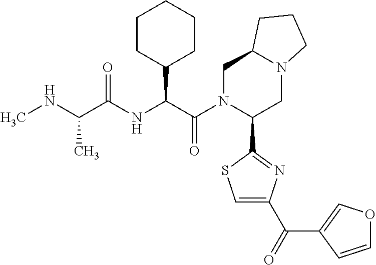

A mixture of S-phenyl 2-{(3S,6aR)-2-[(2S)-2-{[N-(tert-butoxycarbonyl)-N-methyl-L-alanyl]amino}-2-cyclohexylacetyl]octahydropyrrolo[1,2-a]pyrazin-3-yl}-1,3-thiazole-4-carbothioate (50 mg), tris(dibenzylideneacetone) dipalladium(0) (7 mg), furan-3-boronic acid (34 mg), triethyl phosphate (2.5 mg) and copper(I) thiophene-2-carboxylate (22 mg) in dry tetrahydrofuran (2 mL) was stirred under argon at 50° C. overnight. The mixture was diluted with saturated sodium hydrogen carbonate solution (3 mL) and the mixture was extracted with ethyl acetate (2 mL). The extract was filtered through phase separation filter, and the filtrate was concentrated by blowing air at 60° C. The residue was treated with 4M hydrogen chloride-ethyl acetate solution (0.75 mL) in methanol (0.5 mL). After 10 min., the mixture was concentrated by blowing air at room temperature. The obtained residue was purified by preparative HPLC. The desired fraction was concentrated by blowing air at 60° C. to give the title compound (17.8 mg).

TABLE 2

| Ex. | | | |
|---|---|---|---|
| No. | IUPAC name | structure | MS |
| 113 | N-{(1S)-1-cyclohexyl-2- [(3S,8aR)-3-[4-(furan-3- ylcarbonyl)-1,3-thiazol-2- yl]hexahydropyrrolo[1,2- a]pyrazin-2(1H)-yl]-2- oxoethyl}-N-2-methyl-L- alaninamide | 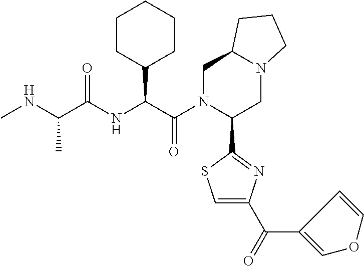 | 528.22 |
| 114 | N-{(1S)-2-[(3S,8aR)-3-[4-(1- benzothiophen-2-ylcarbonyl)- 1,3-thiazol-2- yl]hexahydropyrrolo[1,2- a]pyrazin-2(1H)-yl]-1- cyclohexyl-2-oxoethyl}-N-2- methyl-L-alaninamide | 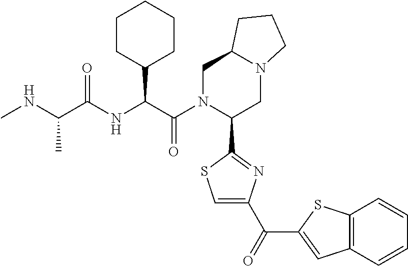 | 594.23 |
| 115 | N-{(1S)-1-cyclohexyl-2-oxo- 2-[(3S,8aR)-3-(4-{[2- (trifluoromethyl)phenyl] carbonyl}-1,3-thiazol-2- yl)hexahydropyrrolo[1,2- a]pyrazin-2(1H)-yl]ethyl}-N- 2-methyl-L-alaninamide | 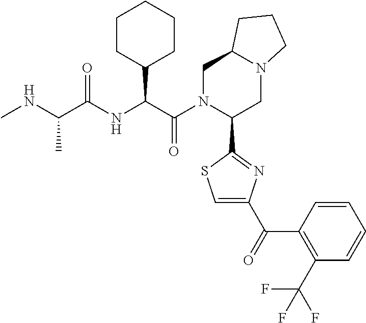 | 606.24 |
| 116 | N-{(1S)-2-[(3S,8aR)-3-[4- (biphenyl-2-ylcarbonyl)-1,3- thiazol-2- yl]hexahydropyrrolo[1,2- a]pyrazin-2(1H)-yl]-1- cyclohexyl-2-oxoethyl}-N-2- methyl-L-alaninamide | 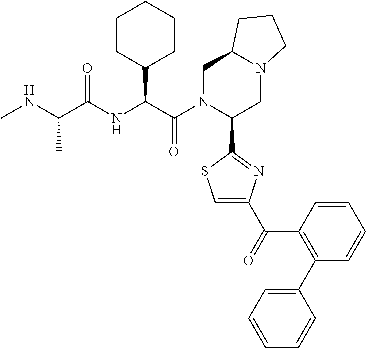 | 614.31 |
| 117 | N-{(1S)-2-[(3S,8aR)-3-[4- (biphenyl-3-ylcarbonyl)-1,3- thiazol-2- yl]hexahydropyrrolo[1,2- a]pyrazin-2(1H)-yl]-1- cyclohexyl-2-oxoethyl}-N-2- methyl-L-alaninamide | 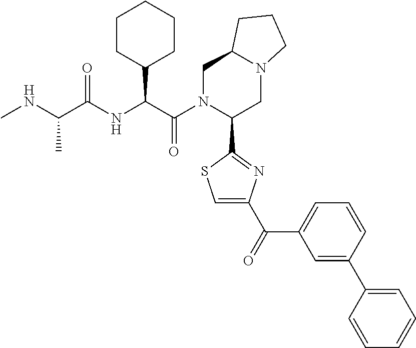 | 614.31 |
| 118 | N-{(1S)-1-cyclohexyl-2-oxo- 2-[(3S,8aR)-3-[4-(2- phenylacryloyl)-1,3-thiazol- 2-yl]hexahydropyrrolo[1,2- a]pyrazin-2(1H)-yl]ethyl}-N- 2-methyl-L-alaninamide | 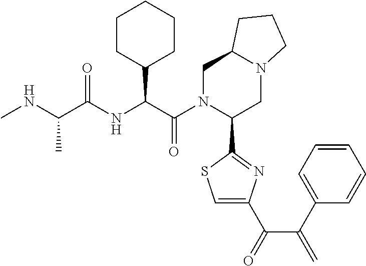 | 564.28 |
| 119 | N-{(2S)-1-cyclohexyl-2- [(3S,8aR)-3-{4-[(1-methyl- 1H-pyrazol-4-yl)carbonyl]- 1,3-thiazol-2- yl}hexahydropyrrolo[1,2- a]pyrazin-2(1H)-yl]-2- oxoethyl}-N-2-methyl-L- alaninamide | 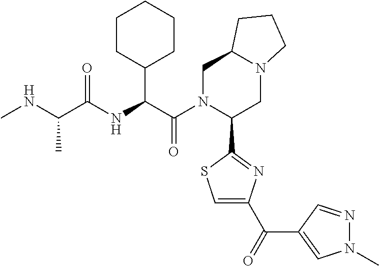 | 542.21 |
| 120 | N-{(1S)-2-[(3S,8aR)-3-{4- [(5-cyanothiophen-2- yl)carbonyl]-1,3-thiazol-2- yl}hexahydropyrrolo[1,2- a]pyrazin-2(1H)-yl]-1- cyclohexyl-2-oxoethyl}-N-2- methyl-L-alaninamide | 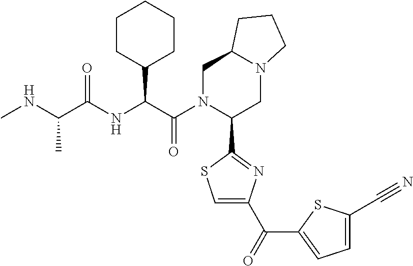 | 569.18 |
| 121 | N-{(1S)-1-cyclohexyl-2-oxo- 2-[(3S,8aR)-3-{4-[(2E)-3- phenylprop-2-enoyl]-1,3- thiazol-2- yl}hexahydropyrrolo[1,2- a]pyrazin-2(1H)-yl]ethyl}-N- 2-methyl-L-alaninamide | 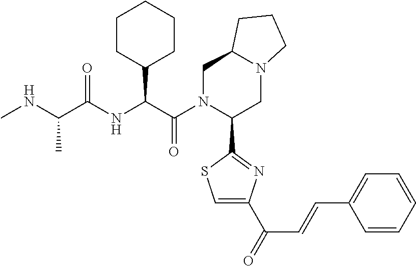 | 564.28 |
| 122 | N-{(1S)-1-cyclohexyl-2- [(3S,8aR)-3-{4-[(3,5- dimethylisoxazol-4- yl)carbonyl]-1,3-thiazol-2- yl}hexahydropyrrolo[1,2- a]pyrazin-2(1H)-yl]-2- oxoethyl}-N-2-methyl-L- alaninamide | 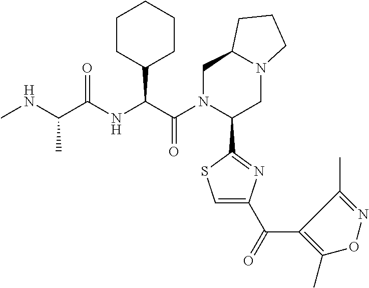 | 557.26 |
| 123 | N-{(1S)-1-cyclohexyl-2- [(3S,8aR)-3-(4-{[3-(2- methylpropoxy)phenyl] carbonyl}-1,3-thiazol-2- yl)hexahydropyrrolo[1,2- a]pyrazin-2(1H)-yl]-2- oxoethyl}-N-2-methyl-L- alaninamide | 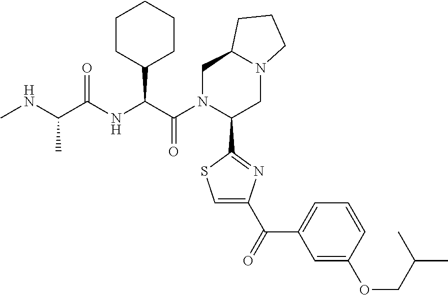 | 610.31 |
| 124 | N-{(1S)-1-cyclohexyl-2-oxo- 2-[(3S,8aR)-3-[4-(pyrimidin- 5-ylcarbonyl)-1,3-thiazol-2- yl]hexahydropyrrolo[1,2- a]pyrazin-2(1H)-yl]ethyl}-N- 2-methyl-L-alaninamide | 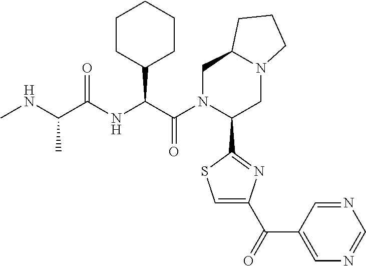 | 540.21 |

### Example 125

### Production of N,N′-bis{(5S)-5-[(N-methyl-L-alanyl)amino]-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}benzene-1,4-dicarboxamide

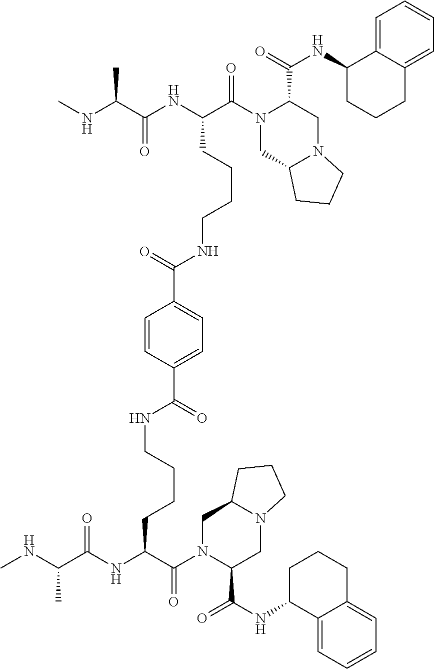

To a mixture of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate (150 mg), terephthalic acid (15.1 mg) and 1-hydroxybenzotriazol (24.6 mg) in N,N-dimethylformamide (5.0 mL) was added 1-ethyl-3-(3-dimethylaminopropyl)carbodiimide hydrochloride (52.3 mg) at 0° C. The reaction mixture was stirred at room temperature for 18 hours. The mixture was diluted with ethyl acetate (30 mL), and washed with water (50 mL), saturated sodium hydrogen carbonate solution (50 mL) and brine (50 mL). The organic layer was dried over anhydrous magnesium sulfate and concentrated in vacuo. The residue was purified by silica gel column chromatography (ethyl acetate/Hexane=10/90-100/0, then ethyl acetate/methanol=80/20) to give a yellow amorphous solid. This yellow amorphous solid was dissolved in a mixed solvent of ethyl acetate (2 mL) and methanol (0.5 mL), and to the solution was added 4M hydrogen chloride-cyclopentyl methyl ether solution (2.0 mL). The mixture was stirred at room temperature for 3 hours. The mixture was extracted with water (20 mL). The aqueous layer was neutralized with 1M sodium hydroxide solution (8 mL) and extracted with ethyl acetate (50 mL). The organic layer was dried over anhydrous magnesium sulfate and concentrated in vacuo. The residue was purified by silica gel column chromatography (ethyl acetate/Hexane=10/90-100/0, then ethyl acetate/MeOH=80/20) to give a yellow amorphous solid. This solid was purified through preparative HPLC [Apparatus: GILSON High Throughput Purification System, Column: YMC combiprep-ODS-A (20 mmI.D.×50 mm, 5 μm), Solvent: phase A: 0.1% trifluoroacetic acid water solution, phase B: 0.1% trifluoroacetic acid acetonitrile solution, Gradient Cycle: 0 min. (phase A/phase B=90/10), 1.20 min. (phase A/phase B=90/10), 7.20 min. (phase A/phase B=60/40), 7.35 min. (phase A/phase B=5/95), 8.50 min. (phase A/phase B=90/10), Detection wavelength: UV 220 nm] to give title compound (4.6 mg) as a colorless amorphous solid.

LC-MS: 1156.6 (MH⁺).

### Example 126

### Production of N,N′-bis{(5S)-5-[(N-methyl-L-alanyl)amino]-6-oxo-6-[(3S,8aR)-3-[(1R)-1,2,3,4-tetrahydronaphthalen-1-ylcarbamoyl]hexahydropyrrolo[1,2-a]pyrazin-2(1H)-yl]hexyl}decanediamide

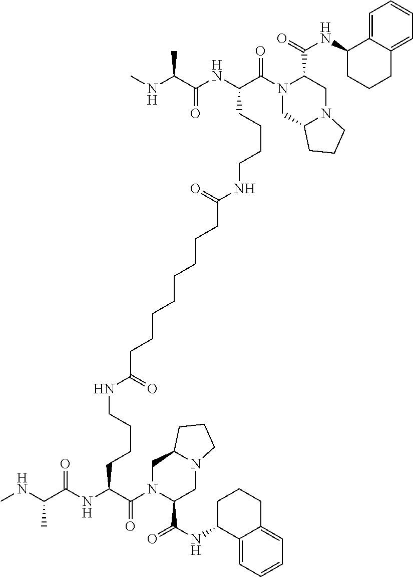

To a mixture of tert-butyl[(1S)-2-{[(1S)-5-amino-1-{[(3S,8aR)-3-[(1R)-1,2,3,4-tetrahydronaphthalen-1-ylcarbamoyl]hexahydropyrrolo[1,2-a]pyrazin-2(1H)-yl]carbonyl}pentyl]amino}-1-methyl-2-oxoethyl]methylcarbamate (100 mg), sebacic acid (12.3 mg) and 1-hydroxybenzotriazol (16.4 mg) in N,N-dimethylformamide (5.0 mL) was added 1-ethyl-3-(3-dimethylaminopropyl)carbodiimide hydrochloride (34.9 mg) at 0° C. The reaction mixture was stirred at room temperature for 18 hours. The mixture was diluted with ethyl acetate (30 mL) and washed with water (50 mL), saturated sodium hydrogen carbonate solution (50 mL), and brine (50 mL). The organic layer was dried over anhydrous magnesium sulfate and concentrated in vacuo. The residue was purified by silica gel column chromatography (ethyl acetate/Hexane=10/90-100/0, then ethyl acetate/methanol=80/20) to give a yellow amorphous solid. This yellow amorphous solid was dissolved in a mixed solvent of ethyl acetate (2 mL) and methanol (0.5 mL), and to the solution was added 4M hydrogen chloride-cyclopentyl methyl ether solution (2.0 mL). The mixture was stirred at room temperature for 3 hours. The mixture was extracted with water (20 mL). The aqueous layer was neutralized with 1M sodium hydroxide solution (8 mL) and extracted with ethyl acetate (50 mL). The organic layer was dried over anhydrous magnesium sulfate and concentrated in vacuo. The residue was purified by silica gel column chromatography (ethyl acetate/Hexane=10/90-100/0, then ethyl acetate/MeOH-80/20) to give a yellow amorphous solid. This solid was purified through preparative HPLC [Apparatus: GILSON High Throughput Purification System, Column: YMC combiprep-ODS-A (20 mmI.D.×50 mm, 5 μm), Solvent: phase A: 0.1% trifluoroacetic acid water solution, phase B: 0.1% trifluoroacetic acid acetonitrile solution, Gradient Cycle: 0 min. (phase A/phase B=90/10), 1.20 min. (phase A/phase B=90/10), 7.20 min. (phase A/phase B=60/40), 7.35 min. (phase A/phase B=5/95), 8.50 min. (phase A/phase B=90/10), Detection wavelength: UV 220 nm] to give title compound (7.0 mg) as a colorless amorphous solid.

LC-MS: 1192.7 (MH⁺).

### Example 127

### Production of (3S,7R,8aR)-2-[(2S)-2-(4,4-difluorocyclohexyl)-2-{[(2S)-2-(methylamino)butanoyl]amino}acetyl]-N-[(4R)-3,4-dihydro-2H-chromen-4-yl]-7-ethoxyoctahydropyrrolo[1,2-a]pyrazine-3-carboxamide

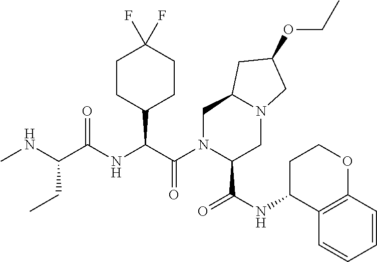

### (i) Production of (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride

A mixture of benzyl{(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamate (1.00 g) and 20% palladium hydroxide on carbon (0.214 g) in 10% hydrogen chloride-methanol solution (40.0 mL) was hydrogenated under balloon pressure at room temperature for 3 hours. The catalyst was removed by filtration and the filtrate was concentrated in vacuo. The residue was diluted with methanol (100 mL) and toluene (100 mL), and the mixture was concentrated in vacuo to give the title compound (1.03 g) as an off-white solid.

LC-MS: 521.1 (MH⁺).

### (ii) Production of tert-butyl[(1S)-1-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamoyl)propyl]methylcarbamate

N,N-Diisopropylethylamine (0.088 mL) was added to a mixture of (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride (100 mg), (S)-2-(tert-butoxycarbonyl(methyl)amino)butanoic acid (43.9 mg), 1-ethyl-3-(3-dimethylaminopropyl)carbodiimide hydrochloride (38.8 mg), and 1-hydroxybenzotriazol (22.77 mg) in N,N-dimethylformamide (1.00 mL) at room temperature. The reaction mixture was stirred at room temperature under dry atmosphere for 19 hours. The mixture was neutralized with saturated sodium hydrogen carbonate solution and extracted twice with ethyl acetate. The combined organic layers were washed with brine, dried over anhydrous magnesium sulfate and concentrated in vacuo. The residue was purified by column chromatography (silica gel, eluted with 2%-75% ethyl acetate in hexane) to give the title compound (68.7 mg) as colorless gum.

LC-MS: 720.2 (MH⁺).

### (iii) Production of (3S,7R,8aR)-2-[(2S)-2-(4,4-difluorocyclohexyl)-2-{[(2S)-2-(methylamino)butanoyl]amino]acetyl}-N-[(4R)-3,4-dihydro-2H-chromen-4-yl]-7-ethoxyoctahydropyrrolo[1,2-a]pyrazine-3-carboxamide

tert-Butyl[(1S)-1-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}carbamoyl)propyl]methylcarbamate (68.7 mg) was dissolved in trifluoroacetic acid (1.00 mL) at room temperature, and the solution was stirred at room temperature for 2 hours. The mixture was concentrated in vacuo, the residue was neutralized with saturated sodium hydrogen carbonate solution, and the mixture was extracted twice with ethyl acetate. The combined organic layers were washed with brine, dried over anhydrous magnesium sulfate and concentrated in vacuo. The residue was crystallized from ethyl acetate-hexane to give the title compound (23.80 mg) as an off-white solid.

LC-MS: 620.1 (MH⁺).

¹H NMR (300 MHz, CDCl₃) δ 0.82-2.45 (28H, m), 2.56-3.25 (3H, m), 3.37 (2H, q, J=6.9 Hz), 3.46-3.84 (1H, m), 3.90-4.38 (4H, m), 4.46-5.45 (3H, m), 6.64-7.96 (6H, m).

### Example 128

### Production of (3S,7R,8aR)-2-{(2S)-2-(4,4-difluorocyclohexyl)-2-[(N-methyl-L-seryl)amino]acetyl}-N-[(4R)-3,4-dihydro-2H-chromen-4-yl]-7-ethoxyoctahydropyrrolo[1,2-a]pyrazine-3-carboxamide

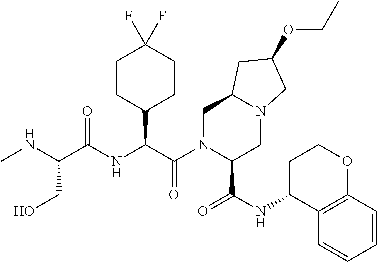

### (i) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(hydroxymethyl)-2-oxoethyl]methylcarbamate

N,N-Diisopropylethylamine (0.059 mL) was added to a mixture of (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride (100 mg), (S)-2-(tert-butoxycarbonyl(methyl)amino)-3-hydroxypropanoic acid (44.3 mg), and 4-(4,6-dimethoxy-1,3,5-triazin-2-yl)-4-methylmorpholin-4-ium chloride (55.9 mg) in a mixed solvent of tetrahydrofuran (2.00 mL)-2-propanol (2.00 mL) at room temperature. The reaction mixture was stirred at room temperature under dry atmosphere for 19 hours. The mixture was concentrated in vacuo, the residue was neutralized with saturated sodium hydrogen carbonate solution, and the mixture was extracted twice with ethyl acetate. The combined organic layers were washed with brine, dried over anhydrous magnesium sulfate and concentrated in vacuo. The residue was purified by column chromatography (silica gel, eluted with 0%-2% methanol in ethyl acetate) to give the title compound (112 mg) as a colorless gum.

LC-MS: 722.2 (MH⁺).

### (ii) Production of (3S,7R,8aR)-2-{(2S)-2-(4,4-difluorocyclohexyl)-2-[(N-methyl-L-seryl)amino]acetyl}-N-[(4R)-3,4-dihydro-2H-chromen-4-yl]-7-ethoxyoctahydropyrrolo[1,2-a]pyrazine-3-carboxamide

tert-Butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(hydroxymethyl)-2-oxoethyl]methylcarbamate (112 mg) was dissolved in trifluoroacetic acid (1.00 mL) at room temperature and the reaction mixture was stirred at room temperature for 2 hours. The mixture was concentrated in vacuo, the residue was neutralized with saturated sodium hydrogen carbonate solution, and the mixture was extracted twice with ethyl acetate. The combined organic layers were washed with brine, dried over anhydrous magnesium sulfate and concentrated in vacuo to give the title compound (46.0 mg) as colorless gum.

LC-MS: 622.1 (MH⁺).

¹H NMR (300 MHz, CDCl₃) δ 1.02-2.75 (26H, m), 2.96-4.36 (10H, m), 4.48-5.38 (3H, m), 6.65-7.00 (3H, m), 7.09-7.24 (2H, m), 7.31-8.23 (1H, m).

### Example 129

### Production of N-{(1S)-1-cyclohexyl-2-oxo-2-[(3S,8aR)-3-[4-(phenylcarbonyl)-1,3-thiazol-2-yl]hexahydropyrrolo[1,2-a]pyrazin-2(1H)-yl]ethyl}-N-2-methyl-L-alaninamide dihydrochloride

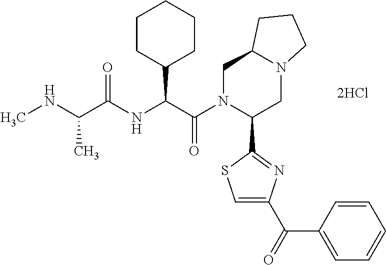

### (i) Production of 2-{(3S,8aR)-2-[(2S)-2-{[N-(tert-butoxycarbonyl)-N-methyl-L-alanyl]amino}-2-cyclohexylacetyl]octahydropyrrolo[1,2-a]pyrazin-3-yl}-1,3-thiazole-4-carboxylic acid

A mixture of tert-butyl(3S,8aR)-3-[4-(ethoxycarbonyl)-1,3-thiazol-2-yl]hexahydropyrrolo[1,2-a]pyrazine-2(1H)-carboxylate (3.11 g) and 4M hydrogen chloride-cyclopentyl methyl ether solution (30 mL) in ethyl acetate (5.0 mL)/methanol (25 mL) was stirred at room temperature for 2.5 hours. The mixture was concentrated in vacuo. The residue was dissolved in N,N-dimethylformamide (20 mL), and to the solution was added (2S)-[(tert-butoxycarbonyl)amino](cyclohexyl)ethanoic acid (2.52 g). After cooling at 0° C., O-(7-azabenzotriazol-1-yl)-N,N,N′,N′-tetramethyluronium hexafluorophosphate (4.65 g) and N,N-diisopropylethylamine (4.25 mL) were added successively to the mixture, and the reaction mixture was stirred at room temperature for 67 hours. The mixture was diluted with sodium hydrogen carbonate solution and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-70/30) to give ethyl 2-[(3S,8aR)-2-{(2S)-2-[(tert-butoxycarbonyl)amino]-2-cyclohexylacetyl}octahydropyrrolo[1,2-a]pyrazin-3-yl]-1,3-thiazole-4-carboxylate including methyl ester derivative (4.12 g) as a pale yellow solid. A mixture of the obtained pale yellow solid (4.12 g) and 4M hydrogen chloride-cyclopentyl methyl ether solution (40 mL) in ethyl acetate (10 mL)/methanol (10 mL) was stirred at room temperature for 1.5 hours. The mixture was concentrated in vacuo. The residue was dissolved in N,N-dimethylformamide (30 mL), and to the solution was added N-(tert-butoxycarbonyl)-N-methyl-L-alanine (1.93 g). After cooling at 0° C., O-(7-azabenzotriazol-1-yl)-N,N,N′,N′-tetramethyluronium hexafluorophosphate (4.51 g) and N,N-diisopropylethylamine (4.13 mL) were added successively to the mixture, and the reaction mixture was stirred at room temperature for 17 hours. The mixture was diluted with sodium hydrogen carbonate solution and extracted twice with ethyl acetate. The combined extracts were washed with brine, dried over anhydrous magnesium sulfate and concentrated in vacuo. The residue was purified by silica gel chromatography (eluent; ethyl acetate/hexane=30/70-100/0) to give ethyl 2-{(3S,8aR)-2-[(2S)-2-{[N-(tert-butoxycarbonyl)-N-methyl-L-alanyl]amino}-2-cyclohexylacetyl]octahydropyrrolo[1,2-a]pyrazin-3-yl}-1,3-thiazole-4-carboxylate including methyl ester derivative (4.83 g) as a pale yellow solid. A mixture of the obtained pale yellow solid (4.79 g) and lithium hydroxide monohydrate (679 mg) in tetrahydrofuran (48 mL)/water (12 mL) was stirred at room temperature for 1.5 hours. The mixture was neutralized with 2N hydrochloric acid and concentrated in vacuo to give the title compound including lithium salt (6.63 g) as a pale yellow solid.

¹H NMR (300 MHz, DMSO-d₆): δ 0.82-1.47 (15H, m), 1.49-2.16 (10H, m), 2.32-2.47 (1H, m), 2.59-2.71 (3H, m), 2.72-2.80 (2H, m), 2.81-3.10 (3H, m), 3.54-3.84 (2H, m), 4.03-4.37 (1H, m), 4.37-4.88 (2H, m), 5.47-6.11 (1

H, m), 7.44-8.09 (1H, m), 8.37 (1H, s).

### (ii) Production of S-phenyl 2-{(3S,8aR)-2-[(2S)-2-{[N-(tert-butoxycarbonyl)-N-methyl-L-alanyl]amino}-2-cyclohexylacetyl]octahydropyrrolo[1,2-a]pyrazin-3-yl}-1,3-thiazole-4-carbothioate

To a mixture of 2-((3S,8aR)-2-((S)-2-((S)-2-(tert-butoxycarbonyl(methyl)amino)propanamido)-2-cyclohexylacetyl)octahydropyrrolo[1,2-a]pyrazin-3-yl)thiazole-4-carboxylic acid (2.60 g) and benzenethiol (0.695 mL) in tetrahydrofuran (50 mL) was added N,N′-dicyclohexylcarbodiimide (1.86 g) at 0° C., and the reaction mixture was stirred at room temperature overnight. The mixture was concentrated in vacuo, and the residue was suspended with ethyl acetate, and insoluble material was filtered off. The filtrate was concentrated in vacuo. The residue was washed with hexane and purified twice by silica gel chromatography (eluent; ethyl acetate/hexane=10/90-100/0) to give the title compound (1.43 g) as a pale yellow solid.

LC-MS: 670.1 (MH⁺).

### (iii) Production of tert-butyl[(1S)-2-({(1S)-1-cyclohexyl-2-oxo-2-[(3S,8aR)-3-[4-(phenylcarbonyl)-1,3-thiazol-2-yl]hexahydropyrrolo[1,2-a]pyrazin-2(1H)-yl]ethyl}amino)-1-methyl-2-oxoethyl]methylcarbamate

A mixture of S-phenyl 2-{(3S,8aR)-2-[(2S)-2-{[N-(tert-butoxycarbonyl)-N-methyl-L-alanyl]amino}-2-cyclohexylacetyl]octahydropyrrolo[1,2-a]pyrazin-3-yl}-1,3-thiazole-4-carbothioate (170 mg), tris(dibenzylideneacetone) dipalladium(0) (23 mg), phenyl boronic acid (62 mg), triethylphosphite (8.5 μL) and copper(I) thiophenecarboxylate (73 mg) in tetrahydrofuran (4.0 mL) was stirred under argon at 50° C. for 6 hours. To the reaction mixture were added additional tris(dibenzylideneacetone) dipalladium(0) (23 mg), phenyl boronic acid (62 mg), triethylphosphite (8.5 μL) and copper(I) thiophenecarboxylate (73 mg), and the reaction mixture was stirred at 50° C. for 4 hours. The mixture was diluted with saturated sodium hydrogen carbonate solution and extracted twice with ethyl acetate. The combined extracts were washed with brine, dried over anhydrous magnesium sulfate and concentrated in vacuo. The residue was purified by silica gel chromatography (eluent; ethyl acetate/hexane=10/90-100/0) to give the title compound (83 mg) as pale brown oil.

LC-MS: 638.4 (MH⁺).

### (iv) Production of N-{(1S)-1-cyclohexyl-2-oxo-2-[(3S,8aR)-3-[4-(phenylcarbonyl)-1,3-thiazol-2-yl]hexahydropyrrolo[1,2-a]pyrazin-2(1H)-yl]ethyl}-N-2-methyl-L-alaninamide dihydrochloride

A mixture of tert-butyl[(1S)-2-({(1S)-1-cyclohexyl-2-oxo-2-[(3S,8aR)-3-[4-(phenylcarbonyl)-1,3-thiazol-2-yl]hexahydropyrrolo[1,2-a]pyrazin-2(1H)-yl]ethyl}amino)-1-methyl-2-oxoethyl]methylcarbamate (108 mg) and 4M hydrogen chloride-ethyl acetate solution (2.0 mL) in ethyl acetate (1.0 mL)/methanol (1.0 mL) was stirred at room temperature for 1.5 hours. The mixture was concentrated in vacuo and the residue was precipitated from methanol-ethyl acetate-diethyl ether to give the title compound (76 mg) as a pale yellow solid.

LC-MS: 538.3 (MH⁺).

¹H NMR (300 MHz, DMSO-d₆): δ 0.78-1.27 (7H, m), 1.28-1.45 (3H, m), 1.46-2.30 (11H, m), 2.89-3.32 (2H, m), 3.48-4.12 (5H, m), 4.47-5.11 (2H, m), 5.58-6.59 (1H, m), 7.35-7.63 (2H, m), 7.63-7.78 (1H, m), 7.90-8.29 (2H, m), 8.45-9.63 (4H, m), 12.09 (1H, br. s.).

### Example 130

### Production of (3S,7R,8aR)-2-{(2S)-2-(4,4-difluorocyclohexyl)-2-[(3-fluoro-N-methyl-D-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

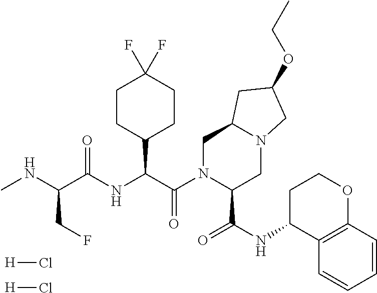

### (i) Production of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(fluoromethyl)-2-oxoethyl]methylcarbamate

1-Ethyl-3-(3-dimethylaminopropyl)carbodiimide (0.313 mL) was added to a mixture of (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride (350 mg), (S)-2-(tert-butoxycarbonyl(methyl)amino)-3-fluoropropanoic acid (143 mg), N,N-diisopropylethylamine (0.257 mL), and 1-hydroxy benzotriazol (88 mg) in N,N-dimethylformamide (10 mL) at room temperature. The mixture was stirred at room temperature under a dry atmosphere for 3 hours. The mixture was poured into water (25 mL) at room temperature and extracted with ethyl acetate (100 mL). The organic layer was separated, washed with water (25 mL) and brine (25 mL), dried over anhydrous magnesium sulfate and concentrated in vacuo. The residue was purified by silica gel column chromatography (eluted with 5%-100% ethyl acetate in hexane) to give the title compound (130 mg) as colorless amorphous solid.

LC-MS: 724.4 (MH⁺).

### (ii) Production of (3S,7R,8aR)-2-{(2S)-2-(4,4-difluorocyclohexyl)-2-[(3-fluoro-N-methyl-D-alanyl)amino]acetyl}-N-[(4R)-3,4-dihydro-2H-chromen-4-yl]-7-ethoxyoctahydropyrrolo[1,2-a]pyrazine-3-carboxamide dihydrochloride

A solution of 4M hydrogen chloride in cyclopentyl methyl ether (7.5 mL) was added dropwise to a solution of tert-butyl[(1S)-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-(fluoromethyl)-2-oxoethyl]methylcarbamate (120 mg) in ethyl acetate (2.5 mL) at room temperature and the mixture was stirred at room temperature for 2 hours. The mixture was concentrated in vacuo and the residue was washed with diethyl ether (20 mL) to give the title compound as colorless amorphous powder.

LC-MS: 624.4 (MH⁺).

¹H NMR (300 MHz, DMSO-d₆): δ 1.03-1.18 (4H, m), 1.19-1.49 (3H, m), 1.50-2.14 (8H, m), 2.15-2.42 (1H, m), 2.57 (3H, brs), 3.41-4.00 (8H, m), 4.01-5.16 (10H, m), 6.69-6.97 (2H, m), 7.03-7.49 (2H, m), 8.46-10.04 (4H, m), 11.59-13.23 (1H, m).

### Reference Example 1

### Production of (2S)-{[(benzyloxy)carbonyl]amino}(4,4-difluorocyclohexyl)ethanoic acid

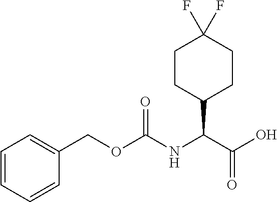

### (i) Production of methyl(2S)-{[(benzyloxy)carbonyl]amino}(4-oxocyclohexyl)ethanoate

Under nitrogen stream, to a mixture of methyl(2S)-{[(benzyloxy)carbonyl]amino}(1,4-dioxaspiro[4.5]dec-8-yl)ethanoate (3.40 g), acetic acid (16 mL), tetrahydrofuran (2.8 mL) and water (5.6 mL) was added dichloroacetic acid (3.57 g), and the mixture was stirred at about 30° C. for 3 hr. 6M Aqueous sodium hydroxide solution (40 mL) was added dropwise thereto at 20° C. or less under ice-cooling to neutralize the mixture (pH 7-8). The obtained solution was extracted with ethyl acetate (100 mL), and the partitioned organic layer was washed with saturated brine (60 mL). The combined organic layer was dried over anhydrous sodium sulfate, and the solvent was concentrated under reduced pressure to give the title compound (2.96 g) as a colorless oil. The same reaction was performed using methyl(2S)-{[(benzyloxy)carbonyl]amino}(1,4-dioxaspiro[4.5]dec-8-yl)ethanoate (64.4 g). The obtained title compound (61.3 g) was combined with the title compound obtained above, and the combined compound was purified by silica gel column (600 g, Fuji silysia BW-820 MH, n-hexane/ethyl acetate=4/1→1/1) to give the title compound (54.2 g) as a colorless oil.

LC-MS: 320.10 (MH⁺)

¹H NMR (CDCl₃, 500 MHz): δ 7.30-7.40 (5H, m), 5.37 (1H, d, J=8.7 Hz), 5.11 (2H, s), 4.48 (1H, dd, J=4.5 Hz, 8.7 Hz), 3.77 (3H, s), 2.39-2.47 (2H, m), 2.24-2.39 (3H, m), 2.02-2.10 (1H, m), 1.88-1.97 (1H, m), 1.50-1.65 (2H, m).

### (ii) Production of methyl(2S)-{[(benzyloxy)carbonyl]amino}(4,4-difluorocyclohexyl)ethanoate

Under nitrogen stream, to a solution of methyl(2S)-{[(benzyloxy)carbonyl]amino}(4-oxocyclohexyl)ethanoate (54.2 g) in dry dichloromethane (310 mL) was added dropwise diethylaminosulfur trifluoride (DAST) (63.0 g) under ice-cooling, and the obtained solution was stirred at room temperature for 1 hr. To the obtained solution was added methanol/water (1/1, 35 mL) under ice-cooling, and the mixture was extracted with water (200 mL), and partitioned. The organic layer was washed with saturated aqueous sodium hydrogen carbonate (250 mL×2) and saturated brine (250 mL), dried over anhydrous sodium sulfate, and concentrated under reduced pressure to give a pale-yellow oil (58.4 g). The obtained pale-yellow oil was dissolved in acetonitrile (150 mL) and water (130 mL), and ruthenium chloride (166 mg) was added thereto. To the obtained suspension was added sodium periodate (25.5 g) under ice-cooling, and the mixture was stirred for 10 min under ice-cooling, and then at room temperature for 5 hr. The mixture was extracted with ethyl acetate and water. The partitioned organic layer was washed with aqueous sodium thiosulfate solution and saturated brine, dried over anhydrous sodium sulfate, and concentrated under reduced pressure. The obtained pale-yellow oil was purified by silica gel column (700 g, Fuji silysia BW-820 MH, n-hexane/ethyl acetate=5/1) to give the title compound (36.1 g) as a white solid.

LC-MS: 342.15 (MH⁺)

¹H NMR (CDCl₃, 500 MHz): δ 7.31-7.40 (5H, m), 5.33 (1H, d, J=8.5 Hz), 5.07-5.15 (2H, m), 4.40 (1H, dd, J=5.0 Hz, 8.5 Hz), 3.76 (3H, s), 2.07-2.17 (2H, m), 1.84-1.94 (1H, m), 1.60-1.81 (4H, m), 1.37-1.51 (2H, m).

### (iii) Production of (2S)-{[(benzyloxy)carbonyl]amino}(4,4-difluorocyclohexyl)ethanoic acid

Under nitrogen stream, methyl(2S)-{[(benzyloxy)carbonyl]amino}(4,4-difluorocyclohexyl)ethanoate (36.1 g) was dissolved in tetrahydrofuran (588 mL) and water (295 mL), and lithium hydroxide monohydrate (17.8 g) was added thereto under ice-cooling. The obtained solution was stirred at about 5° C. for 1 hr, and lithium hydroxide monohydrate (2.92 g) was added thereto. The obtained solution was stirred for 1 hr, and neutralized with 1M hydrochloric acid (405 mL) (pH 4). The obtained reaction mixture was concentrated, 1M hydrochloric acid (105 mL) was added to the obtained residue (pH 2), and the precipitated solid was collected by filtration. The collected solid was dissolved in ethyl acetate (500 mL), and the solution was dried over anhydrous sodium sulfate, and concentrated. The obtained white solid (38.1 g) was subjected to slurry washing with a mixed solvent (350 mL) of n-hexane/ethyl acetate (=100/5) to give the title compound (33.1 g) as a white solid.

¹H NMR (CDCl₃, 500 MHz): δ 12.78 (1H, brs), 7.65 (1H, d, J=8.5 Hz), 7.29-7.40 (5H, m), 5.03 (2H,$), 3.99 (1H, dd, J=6.0 Hz, 8.5 Hz), 1.94-2.05 (2H, m), 1.57-1.93 (5H, m), 1.37-1.48 (1H, m), 1.23-1.35 (1H, m).

### Reference Example 2

### Production of (3S,7R,8aR)-2-[(2S)-2-{[(2S)-2-amino-2-cyclopropylacetyl]amino}-2-cyclohexylacetyl]-N-[(4R)-3,4-dihydro-2H-chromen-4-yl]-7-ethoxyoctahydropyrrolo[1,2-a]pyrazine-3-carboxamide

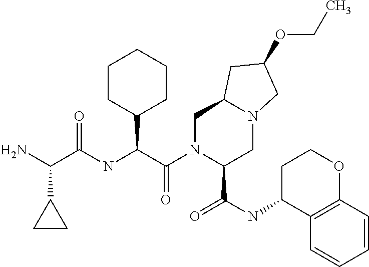

The title compound was synthesized in the same manner as in Examples 85-102.

LC-MS: 582.4 (MH⁺)

### Reference Example 3

### Production of (3S,7R,8aR)-2-[(2S)-2-cyclohexyl-2-{[(2S)-2-cyclopropyl-2-(methylamino)acetyl]amino]acetyl}-N-[(4R)-3,4-dihydro-2H-chromen-4-yl]-7-ethoxyoctahydropyrrolo[1,2-a]pyrazine-3-carboxamide bistrifluoroacetate

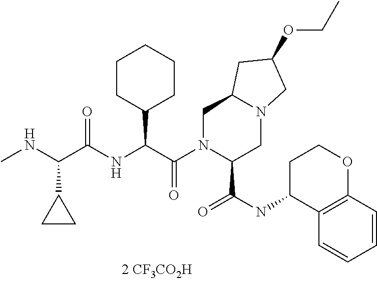

### (i) Production of (S)-2-(tert-butoxycarbonyl(methyl)amino)-2-cyclopropylacetic acid

(S)-2-(tert-Butoxycarbonylamino)-2-cyclopropylacetic acid (750 mg) was dissolved in tetrahydrofuran (15.0 mL), and the solution was cooled to 0° C. Sodium hydride (oil, 60% wt, 348 mg) was added thereto, and the mixture was stirred at 0° C. for 30 min. To the reaction mixture was added methyl iodide (1.09 mL), and the mixture was stirred at room temperature for 22 hr. The reaction mixture was ice-cooled, acidified with 10% aqueous citric acid solution, and extracted twice with ethyl acetate. The organic layers were combined, washed with saturated brine, dried over anhydrous magnesium sulfate, and concentrated under reduced pressure. To the residue was added hexane to elute mineral oil, and the supernatant was removed twice by decantation. The residue was dried under reduced pressure to give the title compound (645 mg) as a pale-yellow oil.

¹H NMR (300 MHz, CDCl₃): δ 0.22-1.32 (5H, m), 1.33-1.62 (9H, m), 3.00 (3H, brs), 3.40-3.98 (1H, m), 5.20 (1H, brs).

### (ii) Production of tert-butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-cyclopropyl-2-oxoethyl]methylcarbamate

(S)-2-(tert-Butoxycarbonyl(methyl)amino)-2-cyclopropylacetic acid (71.0 mg), (3S,7R,8aR)-2-[(2S)-2-amino-2-cyclohexylacetyl]-N-[(4R)-3,4-dihydro-2H-chromen-4-yl]-7-ethoxyoctahydropyrrolo[1,2-a]pyrazine-3-carboxamide (100 mg), 1-ethyl-3-(3-dimethylaminopropyl)carbodiimide hydrochloride (59.3 mg), 1-hydroxybenzotriazole (27.9 mg) and N,N-diisopropylethylamine (0.072 mL) were mixed in N,N-dimethylformamide (1.00 mL), and the mixture was stirred at room temperature for 3 days. To the reaction mixture was added saturated aqueous sodium hydrogen carbonate solution, and the mixture was extracted twice with ethyl acetate. The organic layers were combined, washed with saturated brine, dried over anhydrous magnesium sulfate, and concentrated under reduced pressure. The residue was purified by silica gel column chromatography (ethyl acetate/hexane=2/98→100/0). The obtained object fractions were concentrated under reduced pressure, and the residue was purified again by silica gel column chromatography (ethyl acetate/hexane=50/50→75/25) to give the title compound (28.0 mg) as an oil.

LC-MS: 696.1 (MH⁺).

### (iii) Production of (3S,7R,8aR)-2-[(2S)-2-cyclohexyl-2-{[(2S)-2-cyclopropyl-2-(methylamino)acetyl]amino]acetyl}-N-[(4R)-3,4-dihydro-2H-chromen-4-yl]-7-ethoxyoctahydropyrrolo[1,2-a]pyrazine-3-carboxamide bistrifluoroacetate

tert-Butyl[(1S)-2-({(1S)-1-cyclohexyl-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-1-cyclopropyl-2-oxoethyl]methylcarbamate (28.0 mg) was dissolved in trifluoroacetic acid (1.00 mL), and the solution was stirred at room temperature for 3 hr. The reaction mixture was concentrated under reduced pressure, the residue was diluted with toluene and methanol, and the mixture was concentrated under reduced pressure to give the title compound (38.8 mg).

LC-MS: 596.1 (MH⁺).

¹H NMR (300 MHz, CDCl₃): δ 0.38-2.29 (18H, m), 2.35-2.83 (4H, m), 2.99-3.90 (13H, m), 4.05-5.17 (7H, m), 6.72-6.91 (2H, m), 7.06 (1H, d, J=7.4 Hz), 7.11-7.21 (1H, m), 7.42 (1H, d, J=7.0 Hz), 8.50-12.13 (4H, m).

### Reference Example 4

### Production of (3S,7R,8aR)-2-[(2S)-2-{[(2S)-2-cyclopropyl-2-(methylamino)acetyl]amino}-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide

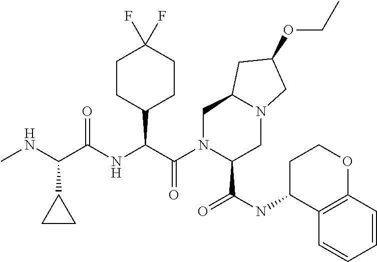

### (i) Production of tert-butyl[(1S)-1-cyclopropyl-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-2-oxoethyl]methylcarbamate

N,N-Diisopropylethylamine (0.088 mL) was added to a mixture of (3S,7R,8aR)-2-[(2S)-2-amino-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide dihydrochloride (100 mg), (S)-2-(tert-butoxycarbonyl(methyl)amino)-2-cyclopropylacetic acid (46.4 mg), 1-ethyl-3-(3-dimethylaminopropyl)carbodiimide hydrochloride (38.8 mg), and 1-hydroxy benzotriazol (22.77 mg) in N,N-dimethylformamide (1.00 mL) at room temperature. The reaction mixture was stirred at room temperature under dry atmosphere for 19 hours. The mixture was neutralized with saturated sodium hydrogen carbonate solution and extracted twice with ethyl acetate. The combined organic layers were washed with brine, dried over anhydrous magnesium sulfate and concentrated in vacuo. The residue was purified by column chromatography (silica gel, eluted with 2%-75% ethyl acetate in hexane) to give the title compound (85 mg) as colorless gum.

LC-MS: 732.2 (MH⁺).

### (ii) Production of (3S,7R,8aR)-2-[(2S)-2-{[(2S)-2-cyclopropyl-2-(methylamino)acetyl]amino}-2-(4,4-difluorocyclohexyl)acetyl]-N-[(4R)-3,4-dihydro-2H-chromen-4-yl]-7-ethoxyoctahydropyrrolo[1,2-a]pyrazine-3-carboxamide

tert-Butyl[(1S)-1-cyclopropyl-2-({(1S)-1-(4,4-difluorocyclohexyl)-2-[(3S,7R,8aR)-3-[(4R)-3,4-dihydro-2H-chromen-4-ylcarbamoyl]-7-ethoxyhexahydropyrrolo[1,2-a]pyrazin-2(1H)-yl]-2-oxoethyl}amino)-2-oxoethyl]methylcarbamate (84.9 mg) was dissolved in trifluoroacetic acid (1.00 mL) at room temperature, and the solution was stirred at room temperature for 2 hours. The reaction mixture was concentrated in vacuo, the residue was neutralized with saturated sodium hydrogen carbonate solution, and the mixture was extracted twice with ethyl acetate. The organic layers were combined, washed with brine, dried over anhydrous magnesium sulfate and concentrated in vacuo. The residue was crystallized from ethyl acetate-hexane to give the title compound (35.4 mg) as an off-white solid.

LC-MS: 632.1 (MH⁺).

¹H NMR (300 MHz, CDCl₃): δ 0.12-2.73 (30H, m), 2.81-5.48 (11H, m), 6.62-7.93 (6H, m).

### Preparation Example 1

A medicament containing the compound of the present invention as an active ingredient can be produced, for example, according to the following formulation.

1. capsule

| | |
|---|---|
| (1) compound of Example 2 | 40 mg |
| (2) lactose | 70 mg |
| (3) crystalline cellulose | 9 mg |
| (4) magnesium stearate | 1 mg |
| 1 capsule | 120 mg |

(1), (2), (3) and 1/2 of (4) are blended and granulated. The rest of (4) is added and the total amount is sealed in a gelatin capsule.

2. tablet

| | |
|---|---|
| (1) compound of Example 2 | 40 mg |
| (2) lactose | 58 mg |
| (3) cornstarch | 18 mg |
| (4) crystalline cellulose | 3.5 mg |
| (5) magnesium stearate | 0.5 mg |
| 1 tablet | 120 mg |

(1), (2), (3), 2/3 of (4) and 1/2 of (5) are blended and granulated. The rest of (4) and (5) is added to the granules and the mixture is compression formed into a tablet.

### Preparation Example 2

The compound (50 mg) obtained in Example 2 is dissolved in the Japanese Pharmacopoeia distilled water for injection (50 mL), and the Japanese Pharmacopoeia distilled water for injection is added to the total amount of 100 mL. This solution is filtered under sterile conditions. The solution (1 mL) is filled under sterile conditions in a vial for injection, freeze-dried and sealed.

The genetic engineering method described below follows the method described in the book (Maniatis et al., Molecular cloning, ColdSpring Harbor Laboratory, 1989) or the method described in a protocol attached to the reagent.

### Experimental Example 1

### Construction of Human cIAP1 (250-350) Expression Vector

Human cIAP1 (250-350) expression plasmid was constructed as follows. PCR was performed by using human brain cDNA library (Takara Bio Inc.) as a template, two kinds of primers (5′-TATTATGGATCCTTGGAAAATTCTCTAGAAACTCTGAGGTTT-3′ (SEQ ID NO: 1) and 5′-TATTATGCGGCCGCTTAAACTTGAATCTCATCAACAAACTCTTG-3′ (SEQ ID NO: 2)) designed by reference to the base sequence described in GenBank accession No. NM_{—}001166, and Pyrobest DNA Polymerase (Takara Bio Inc.). The obtained DNA fragment was digested with restriction enzymes Bam HI and Not I (Takara Bio Inc.), inserted into the downstream of Nde I-Not I site of pET21a (Novagen) together with an initiation codon, His×6 and Bam HI recognition sequence (CATATGCATCATCATCATCATCACGGATCC (SEQ ID NO: 3)) and cloned, whereby human cIAP1 expression plasmid pET21HH/His-hcIAP1 (250-350) was constructed.

### Experimental Example 2

### Expression and Purification of Human cIAP1 (250-350)

Plasmid pET21HH/His-hcIAP1 (250-350) was introduced into Escherichia coli BL21 (DE3) cell (NIPPON GENE CO., LTD.). The transformant was cultured in LB medium (1% trypton, 0.5% yeast extract, and 0.5% NaCl) supplemented with 50 mg/L ampicillin at 37° C., and induced with 1 mM IPTG for 6 hr. His-hBIRC2 (250-350) expression bacterial cells were recovered by centrifugation (6000 rpm, 10 min.) and cryopreserved at −80° C. Frozen cells were thawed on ice, and suspended in a lysis buffer (50 mM Tris-HCl (pH B), 300 mM NaCl, 10% Glycerol, 20 mM imidazole, 1 mM DTT, Complete, EDTA-free (Roche Diagnostics)). To the suspension was added 1 mg/mL lysozyme, and the mixture was stood at 4° C. for 1 hr. Using INSONATOR 201M (KUBOTA), ultrasonic disruption was performed at output 170 W, ON-OFF each 30 sec, 5 repeats, the extract was centrifuged at 40000 rpm, 1 hr, 4° C., and supernatant was recovered. The centrifugation supernatant was filtered through a 0.22 um filter, and applied to Ni-NTA Superflow resin (QIAGEN Inc.) equilibrated with buffer A (50 mM Tris-HCl (pH 8), 300 mM NaCl, 10% Glycerol, 20 mM imidazole, 1 mM DTT). The column was washed with Buffer A, and the object product was eluted with Buffer B (50 mM Tris-HCl (pH 8), 300 mM NaCl, 10% Glycerol, 200 mM imidazole, 1 mM DTT). The obtained eluate was concentrated by Amicon Ultra 15 (3K MWCO, Millipore) and applied to HiLoad 16/60 Superdex200 pg (GE Healthcare) equilibrated with buffer C (50 mM Tris-HCl (pH 8), 300 mM NaCl, 10% Glycerol, 1 mM DTT). Eluted fractions containing the object product were collected, concentrated and cryopreserved at −80° C.

### Experimental Example 3

### Measurement of Inhibitory Activity Against Binding of XIAP or cIAPI

Human XIAP binding inhibitory activity and human cIAP1 binding inhibitory activity were determined by measuring protein-ligand binding inhibition by the compound of the present invention by HTRF method and using commercially available human XIAP_BIR3 domain purified protein (R&D) and human cIAP1(250-350) protein prepared in Experimental Example 2, and Smac N terminal 7 peptide (AVPIAQK (SEQ ID NO: 4)) with C-terminal biotinylated by a conventional method (hereinafter to be referred to as “b-Smac”; PEPTIDE INSTITUTE, INC.) as a ligand. To be specific, human XIAP_BIR3 domain purified protein was diluted to 40 nM, human cIAP1 (250-350) protein was diluted to 40 nM or 8 nM, each with a reaction buffer (25 mM HEPES buffer containing 100 mM NaCl, 0.1% fatty acid-free BSA, 0.1% triton X-100, pH 7.5), and added to a 384-well white shallow bottom plate (Greiner 784075) at 5 μL/well. Then, a test compound diluted with a reaction buffer to each concentration was added at 5 μL/well to the above-mentioned reaction plate. After centrifugation at 1000 rpm for 1 min, b-Smac diluted with the reaction buffer to 20 nM for the measurement of XIAP binding inhibitory activity, or 80 nM or 20 nM for the measurement of cIAP1 binding inhibitory activity (for the measurement of cIAP1 binding inhibitory activity, BO nM b-Smac was used for examination using human cIAP1 (250-350) protein at a concentration of 2 nM during the test, and 20 nM b-Smac was used for examination using human cIAP1 (250-350) protein at a concentration of 10 nM during the test), anti-6HIS-Cryptate (Eu3+ Cryptate-conjugated mouse monoclonal antibody anti-6 Histidine; cisbio) and Streptavidin-XL^{ent!} (Highgrade XL665-conjugated streptavidin; cisbio) each diluted 100-fold with HTRF detection buffer (cisbio) were mixed at 2:1:1 (volume) and the resulting solution was added to the above-mentioned reaction plate at 10 μL/well. The reaction plate was shaken in a plate mixer, centrifuged at 1000 rpm for 1 min, and stood in a dark room at room temperature for 4 hr or longer. The reaction plate was measured using a RUBYstar (BMG LABTECH) at 320 nm excitation wavelength and at 665 nm and 620 nm fluorescence wavelengths. The binding inhibitory rate (%) was calculated from the HTRF ratio (665 nm/620 nm) in the presence of a test compound to that in the absence of the test compound.

The XIAP binding inhibitory rate is shown in Table 3, the cIAP1 binding inhibitory activity measured using 10 nM human cIAP1 (250-350) protein during the test is shown in Table 4, and cIAP1 binding inhibitory activity measured using 2 nM human cIAP1 (250-350) protein is shown in Table 5.

TABLE 3

| | XIAP binding inhibitory rate (%) |
|---|---|
| test compound | at 3 μM of the test compound |
| Example 2 | 98.0 |
| Example 5 | 99.2 |
| Example 6 | 98.8 |
| Example 18 | 99.6 |
| Example 19 | 95.5 |
| Example 24 | 97.3 |
| Example 27 | 97.2 |
| Example 29 | 96.6 |
| Example 41 | 93.5 |
| Example 50 | 97.1 |
| Example 62 | 95.7 |
| Example 80 | 100.2 |
| Example 82 | 96.6 |
| Example 91 | 95.5 |
| Example 125 | 98.9 |
| Example 126 | 99.0 |
| Example 127 | 98.0 |

TABLE 4

| | cIAP1 binding inhibitory rate (%) |
|---|---|
| test compound | at 3 μM of the test compound |
| Example 1 | 95.3 |
| Example 11 | 93.2 |
| Example 13 | 91.5 |

TABLE 5

| | cIAP1 binding inhibitory rate (%) |
|---|---|
| test compound | at 3 μM of the test compound |
| Example 17 | 98.7 |
| Example 23 | 100.5 |
| Example 26 | 98.7 |
| Example 30 | 98.6 |
| Example 32 | 99.4 |
| Example 36 | 99.6 |
| Example 42 | 98.9 |
| Example 47 | 98.4 |
| Example 59 | 99.1 |
| Example 79 | 100.0 |
| Example 88 | 94.1 |
| Example 95 | 93.9 |
| Example 103 | 98.5 |

The above results show that the compound of the present invention has a superior IAP (e.g., XIAP, cIAPI) antagonistic (inhibitory) activity.

### Experimental Example 4

### Construction of Luciferase Expression Vector

Luciferase expression vector (pGL4 CMV-luc/Neo) was prepared by cutting out the necessary portions from pGL4.17[luc2/Neo] (Promega) and pGL4.75[hRluc/CMV] (Promega) and recombining them. To be specific, pGL4.17 and pGL4.75 were digested with restriction enzymes HindIII and SalI, and the DNA treated with the restriction enzymes was electrophoresed on an agarose gel (1.2%). A 3318 bp DNA fragment containing luc2 and Neo was recovered from pGL4.17 and a 2977 bp DNA fragment containing Amp and CMV promoter was recovered from pGL4.75. These were ligated to give expression plasmid pGL4 CMV-luc/Neo. The obtained plasmid was sequenced and the base sequence was confirmed.

### Experimental Example 5

### Preparation of Luciferase Expression MDA-MB-231 Cell (Hereinafter MDA-MB-231-Luc)

MDA-MB-231 cells (American Tissue Culture Collection) were suspended in DMEM medium (Invitrogen) containing 10% fetal bovine serum (Cell Culture Bioscience), plated in a 12-well plate at 1×10⁵ cells/mL, each well 1 mL, and the cells were cultured. After 24 hr of culture, the medium was removed, 100 μL of OMEM (Invitrogen) containing 0.5 mg of pGL4 CMV-luc/Neo plasmid and Lipofectamin LTX (Invitrogen) was added to each well for transfection according to the attached instruction manual. After the plasmid DNA introduction is operation for 25 min., the medium was exchanged with DMEM medium, and the cells were further cultured for 72 hr. The introduced cells were suspended in a medium containing 100 ng/mL G418 (Sigma Ltd.) by a trypsin (Invitrogen) treatment, and plated again in a 6-well plate. The growth state was observed with medium exchange every 3 days, the G418 concentration was increased sequentially, and the cells were cultured for 3 weeks. Finally, MDA-MB-231 cells capable of growing in a medium containing 500 ng/mL G418 were treated by a limit dilution method to give a single colony. Luciferin (Promega) was added to MDA-MB-231 cells in each clone, and the chemical luminescence level was measured using ARVO (PerkinElmer) to give MDA-MB-231-Luc.

### Experimental Example 6

### In Vivo Breast Cancer Cell Tumor Regression Activity

The human breast cancer cell MDA-MB-231-Luc (500 million cells) obtained in Experimental Example 5 were suspended in Matrigel solution (BD Bioscience) and transplanted into 6-week-old BALE/c female nude mouse (CLEA Japan, Inc.) by subcutaneous injection. Mice with a tumor engrafted at a size in tumor volume of about 200 mm³ on day 12-15 post-transplantation were selected and 5 per group were used for the experiment. A 0.5% methylcellulose (Wako Pure Chemical Industries, Ltd.) suspension or 0.1 M PBS solution (NaH₂PO₄/Na₂HPO₄, pH 5.8) of the compound was orally administered at a dose of 100 mg/kg, twice per day for 14 days. The tumor diameter was measured on the day of start of the administration and the day of completion of the administration, and the tumor volume was obtained from formula 1.

tumor volume=long diameter×short diameter×short diameter×(½) formula 1

The ratio of the value obtained by subtracting tumor volume on the day of completion of the administration from that on the day of start of the administration to the tumor volume on the day of start of the administration was obtained as a tumor regression rate from the formula 2.

tumor regression rate (%)=(tumor volume on the day of start of administration−tumor volume on the day of completion of administration)/tumor volume on the day of start of administration×100 formula 2

The tumor regression rate of each test compound is shown in Table 6.

TABLE 6

| Example No. | tumor regression rate (%) |
|---|---|
| 18 | 78 |
| 19 | 64 |
| 23 | 74 |
| 24 | 55 |
| 26 | 41 |
| 32 | 61 |

The above results show that the compound of the present invention has a superior cancer (e.g., breast cancer) treatment effect.

### INDUSTRIAL APPLICABILITY

Since the compound of the present invention shows a superior antagonistic (inhibitory) action on IAP, a clinically useful prophylactic or therapeutic agent for IAP associated diseases (e.g., cancer) can be provided. In addition, since the compound of the present invention is also superior in efficacy expression, pharmacokinetics, solubility, interaction with other pharmaceutical products, and safety such as toxicity and the like and stability, it is useful as a medicament.

This application is based on patent application Nos. 2009-181933 and 2010-003403 filed in Japan, the contents of which are incorporated in full herein by this reference.

1. A compound represented by the formula (I): 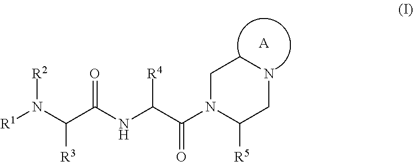
wherein
ring A is a nitrogen-containing heterocycle optionally having substituent(s);
R¹ is a hydrogen atom, a chain aliphatic hydrocarbon group optionally having substituent(s), —OR¹¹, —(═O)—R¹¹, —C(═O)—O—R¹¹, —N(R¹²)—R¹¹ or —C(═O)—N(R¹²)—R¹¹;
R¹¹ is a chain aliphatic hydrocarbon group optionally having substituent(s);
R¹² is a hydrogen atom or a chain aliphatic hydrocarbon group optionally having substituent(s);
R² is a hydrogen atom, a chain aliphatic hydrocarbon group optionally having substituent(s), —C(═O)—R²¹, —C(═O)—O—R²¹ or —C(═O)—N(R²²)—R²¹;
R²¹ is a chain aliphatic hydrocarbon group optionally having substituent(s);
R²² is a hydrogen atom or a chain aliphatic hydrocarbon group optionally having substituent(s);
R³ is a hydrogen atom or a chain aliphatic hydrocarbon group optionally having substituent(s); or
R¹ and R² in combination or R¹ and R³ in combination form a 4- to 7-membered nitrogen-containing non-aromatic heterocycle optionally having substituent(s);
R⁴ is a cyclic group optionally having substituent(s) or a chain aliphatic hydrocarbon group optionally having substituent(s);
R⁵ is a cyclic group optionally having substituent(s), —C(═O)NR⁵¹—X¹—R⁵², C(═S)NR⁵¹—X¹—R⁵², —X²—R⁵², —X²—NR⁵¹R⁵³, —X²—NR⁵¹—C(═O)—R⁵³ or —C(═NR⁵⁴)—NR⁵¹—X¹—R⁵²;
X¹ is a bond or C₁₋₆ alkylene optionally having substituent(s);
X² is C₁₋₆ alkylene optionally having substituent(s);
R⁵¹ is a hydrogen atom, a chain aliphatic hydrocarbon group optionally having substituent(s) or a cyclic group optionally having substituent(s);
R⁵² is a cyclic group optionally having substituent(s);
R⁵³ is a chain aliphatic hydrocarbon group optionally having substituent(s) or a cyclic group optionally having substituent(s); and
R⁵⁴ is a hydrogen atom or a chain aliphatic hydrocarbon group optionally having substituent(s),
or a salt thereof.

2. The compound of claim 1, wherein ring A is a nitrogen-containing heterocycle having C₁₋₆ alkoxy, or a salt thereof.

3. The compound of claim 1, wherein ring A is an unsubstituted nitrogen-containing heterocycle, or a salt thereof.

4. The compound of claim 1, wherein ring A is a 4- to 7-membered monocyclic nitrogen-containing non-aromatic heterocycle optionally having 1 to 5 substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl;
(e) C₃₋₈ cycloalkyloxy;
(f) C₆₋₁₀ aryloxy;
(g) C₇₋₁₃ aralkyloxy; and
(h) oxo,
or a salt thereof.

5. The compound of claim 1, wherein ring A is pyrrolidine optionally having 1 to 5 substituents selected from
(a) a halogen atom;
(b) hydroxy;
(c) C₁₋₆ alkyl optionally having 1 to 3 halogen atoms;
(d) C₁₋₆ alkoxy optionally having 1 to 3 substituents selected from a halogen atom, C₁₋₆ alkoxy and C₃₋₈ cycloalkyl;
(e) C₃₋₈ cycloalkyloxy;
(f) C₅₋₁₀ aryloxy;
(g) C₇₋₁₃ aralkyloxy; and
(h) oxo,
or a salt thereof.

6. The compound of claim 1, wherein R¹ is C₁₋₆ alkyl, or a salt thereof.

7. The compound of claim 1, wherein R² is a hydrogen atom, or a salt thereof.

8. The compound of claim 1, wherein R³ is C₁₋₆ alkyl, or a salt thereof.

9. The compound of claim 1, wherein R⁴ is a cyclic group having halogen atom(s), or a salt thereof.

10. The compound of claim 1, wherein R⁴ is an unsubstituted cyclic group, or a salt thereof.

11. The compound of claim 1, wherein R⁴ is C₃₋₆ cycloalkyl optionally having 1 to 3 substituents selected from
(a) a halogen atom,
(b) hydroxy,
(c) C₁₋₆ alkoxy-carbonyl optionally having one C₆₋₁₀ aryl, and
(d) oxo,
or a salt thereof.

12. The compound of claim 1, wherein R⁵ is
(1) a 5- to 7-membered monocyclic aromatic heterocyclic group optionally having one substituent selected from
(a) C₆₋₁₀ aryl optionally having 1 to 3 halogen atoms,
(b) C₆₋₁₀ aryl-carbonyl optionally having 1 to 3 substituents selected from a halogen atom, C₁₋₆ alkyl optionally having 1 to 3 halogen atoms, C₁₋₆ alkoxy and C₆₋₁₀ aryl,
(c) 5- to 7-membered monocyclic aromatic heterocyclyl-carbonyl optionally having 1 to 3 substituents selected from cyano and C₁₋₆ alkyl,
(d) 8- to 12-membered fused aromatic heterocyclyl-carbonyl,
(e) C₇₋₁₃ aralkyl optionally having 1 to 3 halogen atoms, and
(f) C₂₋₆ alkenyl-carbonyl optionally having one C₆₋₁₀ aryl;
(2) a 8- to 12-membered fused aromatic heterocyclic group optionally having one substituent selected from
(a) C₆₋₁₀ aryl optionally having 1 to 3 halogen atoms,
(b) C₆₋₁₀ aryl-carbonyl optionally having 1 to 3 substituents selected from a halogen atom, C₁₋₆ alkyl optionally having 1 to 3 halogen atoms, C₁₋₆ alkoxy and C₆₋₁₀ aryl,
(c) 5- to 7-membered monocyclic aromatic heterocyclyl-carbonyl optionally having 1 to 3 substituents selected from cyano and C₁₋₆ alkyl,
(d) 8- to 12-membered fused aromatic heterocyclyl-carbonyl,
(e) C₇₋₁₃ aralkyl optionally having 1 to 3 halogen atoms, and
(f) C₂₋₆ alkenyl-carbonyl optionally having one C₆₋₁₀ aryl;
(3) —C(═O)NR⁵¹—X¹—R⁵² wherein
R⁵¹ is a hydrogen atom,
R⁵² is
(a) a monovalent group derived from a fused ring formed by condensation of C₃₋₆ cycloalkane and a benzene ring, which optionally has 1 to 3 substituents selected from hydroxy, a halogen atom, C₁₋₆ alkyl-carbonyloxy and oxo;
(b) a monovalent group derived from a fused ring formed by condensation of tetrahydropyran or tetrahydrofuran and a benzene ring, which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group optionally having one C₆₋₁₀ aryl; or
(e) a 8- to 12-membered fused non-aromatic heterocyclic group, and
X¹ is
(a) a bond, or
(b) C₁₋₆ alkylene optionally having 1 to 3 substituents selected from C₆₋₁₀ aryl and a halogen atom;
(4) —X²—R⁵² wherein
R⁵² is a 8- to 12-membered fused non-aromatic heterocyclic group, and
X² is C₁₋₆ alkylene; or
(5) —X²—NR⁵¹R⁵³ wherein
R⁵¹ is a hydrogen atom,
R⁵³ is a monovalent group derived from a fused ring formed by condensation of C₃₋₆ cycloalkane and a benzene ring, and
X² is C₁₋₆ alkylene;
or a salt thereof.

13. The compound of claim 1, wherein R⁵ is —C(═O)NR⁵¹—X¹—R⁵² wherein
R⁵¹ is a hydrogen atom,
R⁵² is
(a) a monovalent group derived from a fused ring formed by condensation of C₃₋₈ cycloalkane and a benzene ring, which optionally has 1 to 3 substituents selected from hydroxy, a halogen atom, C₁₋₆ alkyl-carbonyloxy and oxo;
(b) a monovalent group derived from a fused ring formed by condensation of tetrahydropyran or tetrahydrofuran and a benzene ring, which optionally has 1 to 3 halogen atoms;
(c) C₆₋₁₀ aryl optionally having 1 to 3 C₁₋₆ alkyl;
(d) a 5- to 7-membered monocyclic aromatic heterocyclic group optionally having one C₆₋₁₀ aryl; or
(e) dihydroindolyl, and
X¹ is
(a) a bond, or
(b) C₁₋₆ alkylene optionally having 1 to 3 substituents selected from C₆₋₁₀ aryl and a halogen atom;
or a salt thereof.

14. The compound of claim 1, wherein
ring A is a 4- to 7-membered monocyclic nitrogen-containing non-aromatic heterocycle optionally having substituent(s) or a 6- to 12-membered fused nitrogen-containing non-aromatic heterocycle optionally having substituent(s),
R¹ is a hydrogen atom, C₁₋₆ alkyl optionally having substituent(s) or —OR¹¹ wherein R¹¹ is defined as in claim 1,
R² is a hydrogen atom or C₁₋₆ alkyl optionally having substituent(s),
R³ is a hydrogen atom, C₁₋₆ alkyl optionally having substituent(s) or C₃₋₈ cycloalkyl optionally having substituent(s), or
R¹ and R³ in combination form, together with the adjacent nitrogen atom and carbon atom, a 4- to 7-membered nitrogen-containing non-aromatic heterocycle optionally having substituent(s),
R⁴ is
(1) C₃₋₈ cycloalkyl optionally having substituent(s),
(2) a 4- to 7-membered monocyclic non-aromatic heterocyclic group optionally having substituent(s),
(3) C₁₋₆ alkyl optionally having substituent(s), or
(4) C₆₋₁₀ aryl optionally having substituent(s),
R⁵ is
(1) a 5- to 7-membered monocyclic aromatic heterocyclic group optionally having substituent(s),
(2) a 8- to 12-membered fused aromatic heterocyclic group optionally having substituent(s),
(3) —C(═O)NR⁵¹—X¹—R⁵² wherein R⁵¹, X¹ and R⁵² are defined as in claim 1,
(4) —X²—R⁵² wherein R⁵² and X² are defined as in claim 1, or
(5) —X²—NR⁵¹R⁵³ wherein R⁵¹, X² and R⁵³ are defined as in claim 1,
or a salt thereof.

15. A compound represented by the formula (II): 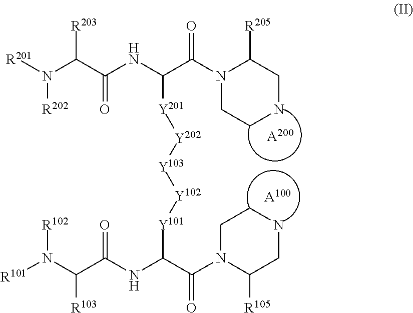
wherein
ring A¹⁰⁰ is a nitrogen-containing heterocycle optionally having substituent(s);
R¹⁰¹ is a hydrogen atom, a chain aliphatic hydrocarbon group optionally having substituent(s), —OR¹¹¹, —C(═O)—O—R¹¹¹, —N(R¹¹²)—R¹¹¹ or —C(═O)—N(R¹¹²)—R¹¹¹;
R¹¹¹ is a chain aliphatic hydrocarbon group optionally having substituent(s);
R¹¹² is a hydrogen atom or a chain aliphatic hydrocarbon group optionally having substituent(s);
R¹⁰² is a hydrogen atom, a chain aliphatic hydrocarbon group optionally having substituent(s), —C(═O)—R¹²¹, —C(═O)—O—R¹²¹ or —C(═O)—N(R¹²²)—R¹²¹;
R¹²¹ is a chain aliphatic hydrocarbon group optionally having substituent(s);
R¹²² is a hydrogen atom or a chain aliphatic hydrocarbon group optionally having substituent(s);
R¹⁰³ is a hydrogen atom or a chain aliphatic hydrocarbon group optionally having substituent(s); or
R¹⁰¹ and R¹⁰² in combination or R¹⁰¹ and R¹⁰³ in combination form a 4- to 7-membered nitrogen-containing non-aromatic heterocycle optionally having substituent(s);
R¹⁰⁵ is a cyclic group optionally having substituent(s), —C(═O)—NR¹⁵¹—X¹⁰¹—R¹⁵², —C(═S)—NR¹⁵¹—X¹⁰¹—R¹⁵², —X¹⁰²—R¹⁵², X¹⁰²—NR¹⁵¹R¹⁵³, —X¹⁰²—NR¹⁵¹—C(═O)—R¹⁵³ or —C(═NR¹⁵⁴)—NR¹⁵¹—X¹⁰¹—R¹⁵²;
X¹⁰¹ is a bond or C₁₋₆ alkylene optionally having substituent(s);
X¹⁰² is C₁₋₆ alkylene optionally having substituent(s);
R¹⁵¹ is a hydrogen atom, a chain aliphatic hydrocarbon group optionally having substituent(s) or a cyclic group optionally having substituent(s);
R¹⁵² is a cyclic group optionally having substituent(s);
R¹⁵³ is a chain aliphatic hydrocarbon group optionally having substituent(s) or a cyclic group optionally having substituent(s);
R¹⁵⁴ is a hydrogen atom or a chain aliphatic hydrocarbon group optionally having substituent(s);
—Y¹⁰¹— is a divalent chain aliphatic hydrocarbon group optionally having substituent(s) or a divalent cyclic group optionally having substituent(s);
—Y¹⁰²— is a bond, —C(═O)—, —C(═O)—O—, —C(═O)—NR¹⁰⁶—, —O—, —O—C(═O)—, —O—C(═O)—NR¹⁰⁶—, —NR¹⁰⁶—, —NR¹⁰⁶—C(═O)—, —NR¹⁰⁶—S(═O)₂—, —S—, —S(═O)—, —S(═O)₂— or —S(═O)₂—NR¹⁰⁶—;
R¹⁰⁶ is a hydrogen atom or a chain aliphatic hydrocarbon group optionally having substituent(s);
—Y¹⁰³— is a bond or a spacer having 1 to 20 atoms in the main chain;
ring A²⁰⁰ is a nitrogen-containing heterocycle optionally having substituent(s);
R²⁰¹ is a hydrogen atom, a chain aliphatic hydrocarbon group optionally having substituent(s), —OR²¹¹, —C(═O)—R²¹¹, —C(═O)—O—R²¹¹, —N(R²¹²)—R²¹¹ or —C(═O)—N(R²¹²)—R²¹¹;
R²¹¹ is a chain aliphatic hydrocarbon group optionally having substituent(s);
R²¹² is a hydrogen atom or a chain aliphatic hydrocarbon group optionally having substituent(s);
R²⁰² is a hydrogen atom, a chain aliphatic hydrocarbon group optionally having substituent(s), —C(═O)—R²²¹, —C(═O)—O—R²²¹ or —C(═O)—N(R²²²)—R²²¹;
R²²¹ is a chain aliphatic hydrocarbon group optionally having substituent(s);
R²²² is a hydrogen atom or a chain aliphatic hydrocarbon group optionally having substituent(s);
R²⁰³ is a hydrogen atom or a chain aliphatic hydrocarbon group optionally having substituent(s); or
R²⁰¹ and R²⁰² in combination or R²⁰¹ and R²⁰³ in combination form a 4- to 7-membered nitrogen-containing non-aromatic heterocycle optionally having substituent(s);
R²⁰⁵ is a cyclic group optionally having substituent(s), —C(═O)—NR²⁵¹—X²⁰¹—R²⁵², —C(═S)—NR²⁵¹—X²⁰¹—R²⁵², —X²⁰²—R²⁵², —X²⁰²—NR²⁵¹R²⁵³, —X²⁰²—NR²⁵¹—C(═O)—R²⁵³ or —C(═NR²⁵⁴)—NR²⁵¹—X²⁰¹—R²⁵²;
X²⁰¹ is a bond or C₁₋₆ alkylene optionally having substituent(s);
X²⁰² is C₁₋₆ alkylene optionally having substituent(s);
R²⁵¹ is a hydrogen atom, a chain aliphatic hydrocarbon group optionally having substituent(s) or a cyclic group optionally having substituent(s);
R²⁵² is a cyclic group optionally having substituent(s);
R²⁵³ is a chain aliphatic hydrocarbon group optionally having substituent(s) or a cyclic group optionally having substituent(s);
R²⁵⁴ is a hydrogen atom or a chain aliphatic hydrocarbon group optionally having substituent(s);
—Y²⁰¹— is a divalent chain aliphatic hydrocarbon group optionally having substituent(s) or a divalent cyclic group optionally having substituent(s);
—Y²⁰²— is a bond, —C(═O)—, —C(═O)—O—, —C(═O)—NR²⁰⁶—, —O—, —O—C(═O)—, —O—C(═O)—NR²⁰⁶—, —NR²⁰⁶—, —NR²⁰⁶—C(═O)—O—, —NR²⁰⁶—S(═O)₂—, —S—, —S(═O)—, —S(═O)₂— or —S(═O)₂—NR²⁰⁶—;
R²⁰⁶ is a hydrogen atom or a chain aliphatic hydrocarbon group optionally having substituent(s),
or a salt thereof.

16. A compound which is (3S,7R,8aR)-2-{(2S)-2-cyclohexyl-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide, or a salt thereof.

17. A compound which is (3S,8aR)—N-[(4R)-3,4-dihydro-2H-chromen-4-yl]-2-{(2S)-2-[(N-methyl-L-alanyl)amino]-2-phenylacetyl}octahydropyrrolo[1,2-a]pyrazine-3-carboxamide, or a salt thereof.

18. A compound which is (3S,7R,8aR)—N-[(4R)-3,4-dihydro-2H-chromen-4-yl]-7-ethoxy-2-{(2S)-2-[(N-methyl-L-alanyl)amino]-2-phenylacetyl}octahydropyrrolo[1,2-a]pyrazine-3-carboxamide, or a salt thereof.

19. A compound which is (3S,7R,8aR)-2-{(2S)-2-(4,4-difluorocyclohexyl)-2-[(N-methyl-L-alanyl)amino]acetyl}-N-[(4R)-3,4-dihydro-2H-chromen-4-yl]-7-ethoxyoctahydropyrrolo[1,2-a]pyrazine-3-carboxamide, or a salt thereof.

20. A compound which is (3S,8aR)-2-{(2S)-2-cyclohexyl-2-[(N-methyl-L-alanyl)amino]acetyl}-N-[(4R)-3,4-dihydro-2H-chromen-4-yl]octahydropyrrolo[1,2-a]pyrazine-3-carboxamide, or a salt thereof.

21. A compound which is (3S,8aR)-2-{(2S)-2-(4,4-difluorocyclohexyl)-2-[(N-methyl-L-alanyl)amino]acetyl}-N-[(4R)-3,4-dihydro-2H-chromen-4-yl]octahydropyrrolo[1,2-a]pyrazine-3-carboxamide, or a salt thereof.

22. A prodrug of the compound of claim 1 or a salt thereof.

23. A medicament comprising the compound of claim 1 or a salt thereof or a prodrug thereof.

24. The medicament of claim 23, which is an inhibitor of apoptosis proteins.

25. The medicament of claim 23, which is an agent for the prophylaxis or treatment of cancer.

26. A method of antagonizing an inhibitor of apoptosis proteins in a mammal, which comprises administering an effective amount of the compound of claim 1 or a salt thereof or a prodrug thereof to the mammal.

27. A method for the prophylaxis or treatment of cancer in a mammal, which comprises administering an effective amount of the compound of claim 1 or a salt thereof or a prodrug thereof to the mammal.

28. Use of the compound of claim 1 or a salt thereof or a prodrug thereof for the production of an inhibitor of apoptosis proteins.

29. Use of the compound of claim 1 or a salt thereof or a prodrug thereof for the production of an agent for the prophylaxis or treatment of cancer.


SEQUENCE LISTING (4 sequences)

SEQ ID NO: 1 - DNA, 42 bp, Artificial
  Designed oligonucleotide primer
tattatggatccttggaaaattctctagaaactctgaggttt

SEQ ID NO: 2 - DNA, 44 bp, Artificial
  Designed oligonucleotide primer
tattatgcggccgcttaaacttgaatctcatcaacaaactcttg

SEQ ID NO: 3 - DNA, 30 bp, Artificial
  Designed oligonucleotide for cloning
catatgcatcatcatcatcatcacggatcc

SEQ ID NO: 4 - protein, 7 aa, Artificial
  N-terminal peptides of Smac
AVPIAQK